(19) United States
(12) Patent Application Publication
(10) Pub. No.: US 2004/0052799 A1
(43) Pub. Date: Mar. 18, 2004

(54) Nucleic acid and amino acid sequences relating to Helicobacter pylori for diagnostics and therapeutics

(75) Inventors: Douglas Smith, Gloucester, MA (US); Richard A. Alm, Ashland, MA (US); O. Thomas Berglindh, Uppsala (SE); Bjorn L. Mellgard, Goteborg (SE); Steven R. Thomas, Mission (CA); Eric Brown, Cambridge, MA (US); Donald T. Moir, Lexington, MA (US); Lo See Ling, Arlington, MA (US); Gilles Carmel, Waltham, MA (US); Braydon C. Guild, Concord, MA (US); Peter C. Doig, Acton, MA (US); Zita Kabok, Natick, MA (US); Lillian Marie Castriotta, Newton, MA (US)

Correspondence Address: LAHIVE & COCKFIELD, LLP., 28 STATE STREET, BOSTON, MA, 02109, US

(73) Assignee: Astra Aktiebolag

(21) Appl. No.: 10/335,977

(22) Filed: Dec. 30, 2002

Related U.S. Application Data
(63) Continuation of application No. 08/993,002, filed on Dec. 17, 1997, now abandoned.
Application No. 08/993,002 is a continuation-in-part of application No. 08/821,931, filed on Mar. 21, 1997, now abandoned.
Application No. 08/821,931 is a continuation of application No. 08/761,184, filed on Dec. 5, 1996, now abandoned.

(30) Foreign Application Priority Data
Oct. 28, 1997 (WO) PCT/US97/19575
Dec. 5, 1997 (WO) PCT/US97/22104
Nov. 15, 1996 (WO) PCT/US96/18542

(51) Int. Cl. A61K 39/00; A61K 39/38

(52) U.S. Cl. 424/184.1

(57) ABSTRACT

Recombinant or substantially pure preparations of H. pylori polypeptides are described. The nucleic acids encoding the polypeptides also are described. The H. pylori polypeptides are useful for diagnostics and vaccine compositions.

### RELATED APPLICATIONS

The contents, including the Sequence Listings and Figures, of each of the following related applications listed by serial number and filing date is incorporated herein by reference.

This application is a continuation-in-part of U.S. Ser. No. 08/821,931, filed Mar. 21, 1997, which is a continuation of U.S. Ser. No. 08/761,184, filed Dec. 6, 1996 now abandoned, which is a continuation-in-part of U.S. Ser. No. 08/759,739, filed Dec. 6, 1996, which is a continuation-in-part of U.S. Ser. No. 08/736,791, filed Oct. 25, 1996, which is a continuation-in-part of U.S. Ser. No. 08/739,150, filed Oct. 28, 1996, which is a continuation-in-part of 08/660,742, filed Jun. 6, 1996, which is a continuation-in-part of U.S. Ser. No. 08/630,405, filed Apr. 1, 1996, which is a continuation-in-part of U.S. Ser. No. 08/561,469, filed Nov. 17, 1995, which is a continuation-in-part of U.S. Ser. No. 08/487,032, filed Jun. 7, 1995. This application also claims priority to PCT application PCT/US97/19575, filed Oct. 28, 1997, and PCT application PCT/US96/18542, filed Nov. 15, 1996. This application is also a continuation-in-part of U.S. Ser. No. 08/625,431 filed Mar. 26, 1996 which is a continuation-in-part of U.S. Ser. No. 08/621,425, filed Mar. 25, 1996, which is a continuation-in-part of U.S. Ser. No. 08/561,469, filed Nov. 17, 1995, which is a continuation-in-part of U.S. Ser. No. 08/487,032, filed Jun. 7, 1995. This application is also a continuation-in-part of U.S. Ser. No. 08/824,132, filed Mar. 27, 1997, which is a continuation-in-part of U.S. Ser. No. 08/761,318, filed Dec. 6, 1996, which is a continuation-in-part of U.S. Ser. No. 08/738,859, filed Oct. 28, 1996 now abandoned, which is a continuation-in-part of U.S. Ser. No. 08/625,811, filed Mar. 29, 1996. This application is also a continuation-in-part of U.S. Ser. No. 08/769,224, filed Dec. 6, 1996, which is a continuation-in-part of U.S. Ser. No. 08/736,905, filed Oct. 25, 1996 now abandoned, which is a continuation-in-part of U.S. Ser. No. 08/758,731, filed Apr. 2, 1996. This application is also a continuation-in-part of U.S. Ser. No. 08/823,745, filed Mar. 25, 1997, which is a continuation-in-part of U.S. Ser. No. 08/759,625, filed Dec. 5, 1996. This application also claims priority to a PCT application, number not yet available, filed Dec. 5, 1997, (Atty. Docket No. GTN-011CP2PC). This application is also a continuation-in-part of U.S. Ser. No. 08/761,066, filed Dec. 5, 1996. This application is also a continuation-in-part of U.S. Ser. No. 08/891,928, filed Jul. 14, 1997. This application is also a continuation-in-part of U.S. Ser. No. 08/892,020, filed Jul. 14, 1997.

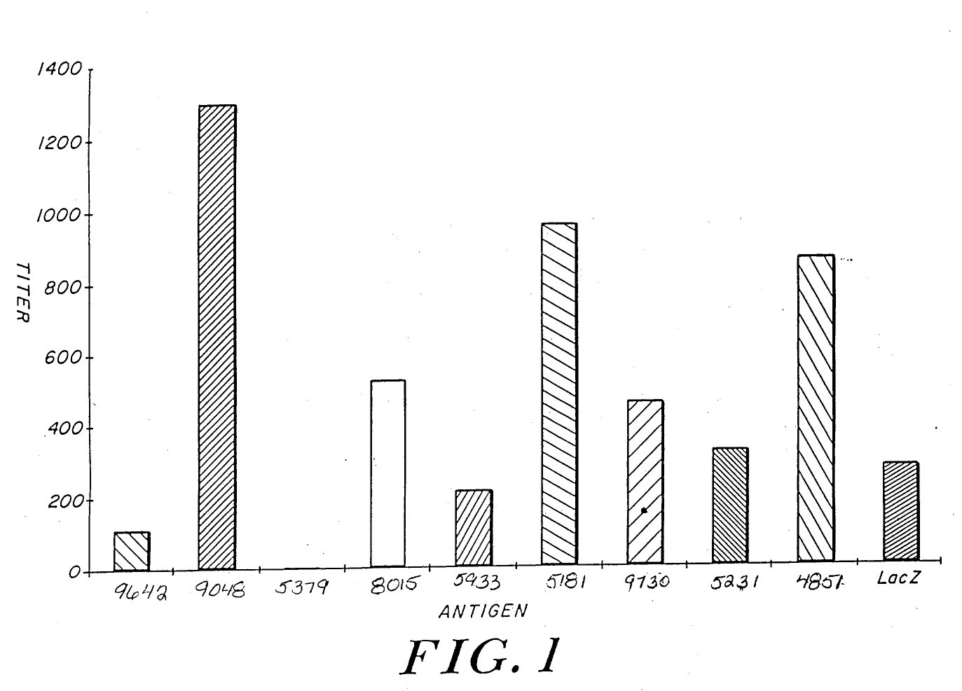

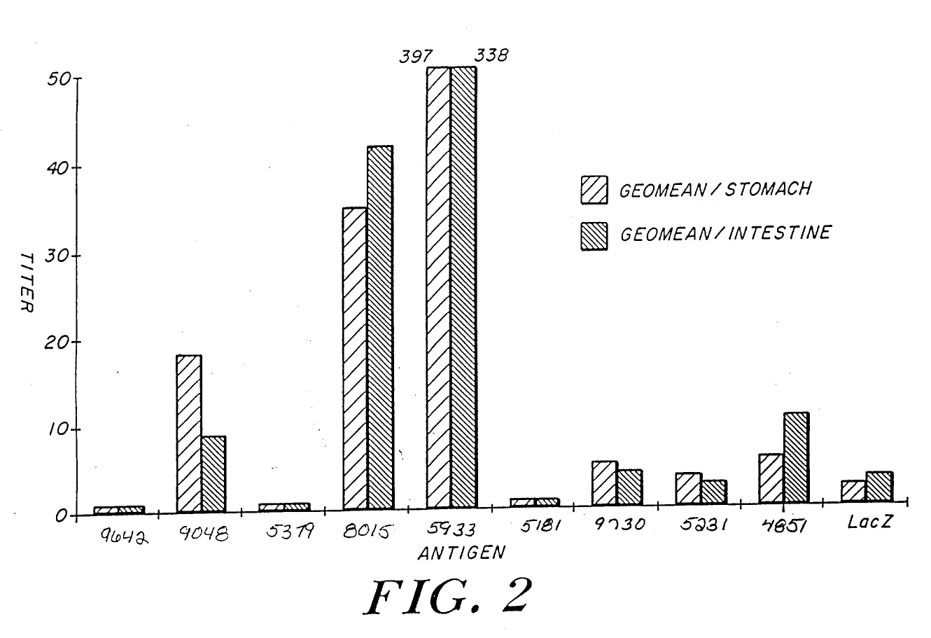

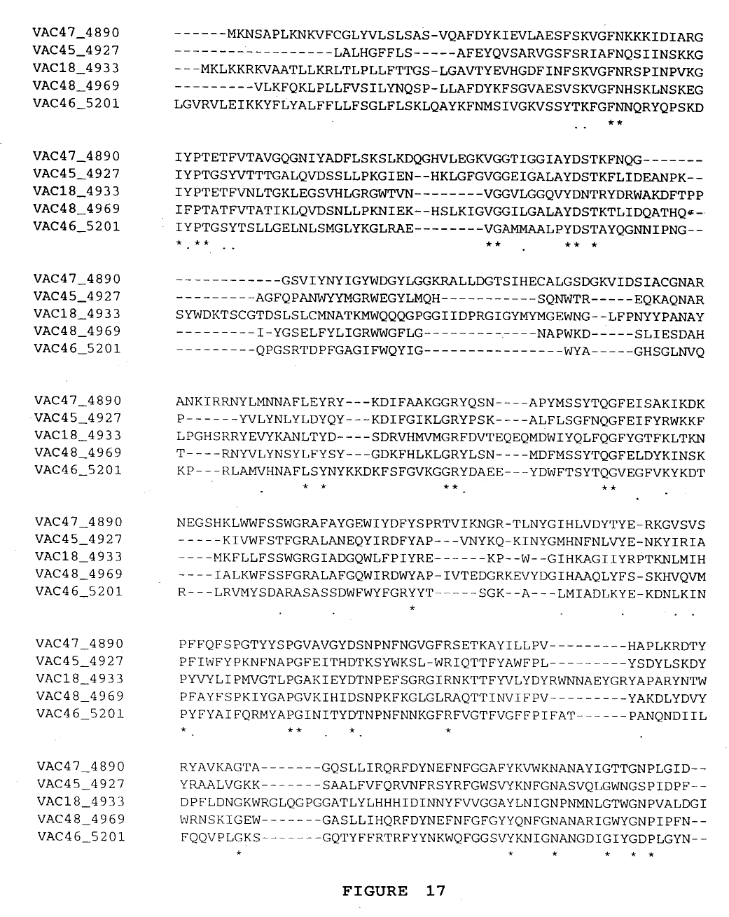

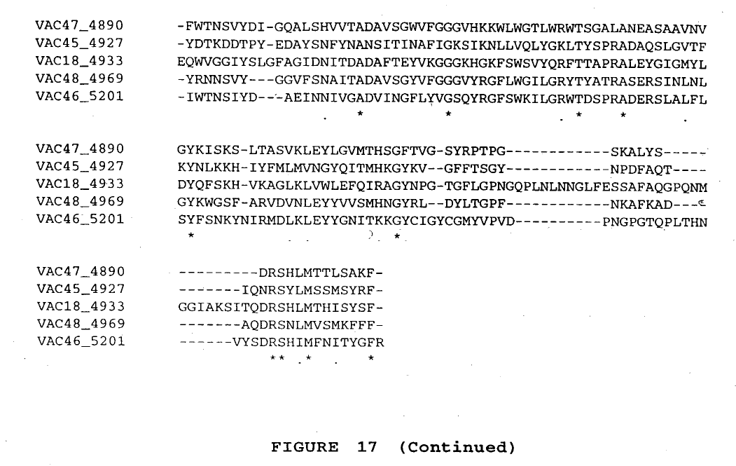

It should be understood that all of the Sequence Listings and Figures of all of the aforementioned applications are considered to be part of the contents which are incorporated by reference herein. For example, FIG. 1 (pages 1-1199), which contains H. pylori genomic DNA, of U.S. Ser. No. 08/621,425, filed Mar. 25, 1996 is incorporated herein by reference. FIG. 1 (pages 1-1199), which contains H. pylori genomic DNA, and FIGS. 2-809 and FIG. 810 (pages 908-2700), which contain H. pylori amino acid sequences, of U.S. Ser. No. 08/625,431, filed Mar. 26, 1996 also are incorporated herein by reference.

### BACKGROUND OF THE INVENTION

Helicobacter pylori is a gram-negative, S-shaped, microaerophilic bacterium that was discovered and cultured from a human gastric biopsy specimen. (Warren, J. R. and B. Marshall, (1983) Lancet 1: 1273-1275; and Marshall et al., (1984) Microbios Lett. 25: 83-88). H. pylori has been strongly linked to chronic gastritis and duodenal ulcer disease. (Rathbone et. al., (1986) Gut 27: 635-641). Moreover, evidence is accumulating for an etiologic role of H. pylori in nonulcer dyspepsia, gastric ulcer disease, and gastric adenocarcinoma. (Blaser M. J., (1993) Trends Microbiol. 1: 255-260). Transmission of the bacteria occurs via the oral route, and the risk of infection increases with age. (Taylor, D. N. and M. J. Blaser, (1991) Epidemiol. Rev 13: 42-50). H. pylori colonizes the human gastric mucosa, establishing an infection that usually persists for decades. Infection by H. pylori is prevalent worldwide. Developed countries have infection rates over 50% of the adult population, while developing countries have infection rates reaching 90% of the adults over the age of 20. (Hopkins R. J. and J. G. Morris (1994) Am. J. Med. 97: 265-277).

The bacterial factors necessary for colonization of the gastric environment, and for virulence of this pathogen, are poorly understood. Examples of the putative virulence factors include the following: urease, an enzyme that may play a role in neutralizing gastric acid pH (Eaton et al., (1991) Infect. Immunol. 59: 2470-2475; Ferrero, R. L. and A. Lee (1991) Microb. Ecol. Hlth. Dis. 4: 121-134; Labigne et al., (1991) J. Bacteriol. 173: 1920-1931); the bacterial flagellar proteins responsible for motility across the mucous layer. (Hazell et al., (1986) J. Inf. Dis. 153: 658-663; Leying et al., (1992) Mol. Microbiol. 6: 2863-2874; and Haas et al., (1993) Mol. Microbiol. 8: 753-760); Vac A, a bacterial toxin that induces the formation of intracellular vacuoles in epithelial cells (Schmitt, W. and R. Haas, (1994) Molecular Microbiol. 12(2): 307-319); and several gastric tissue-specific adhesins. (Boren et al., (1993) Science 262: 1892-1895; Evans et al., (1993) J. Bacteriol. 175: 674-683; and Falk et al., (1993) Proc. Natl. Acad. Sci. USA 90: 2035-203).

Numerous therapeutic agents are currently available that eradicate H. pylori infections in vitro. (Huesca et. al., (1993) Zbl. Bakt. 280: 244-252; Hopkins, R. J. and J. G. Morris, supra). However, many of these treatments are suboptimally effective in vivo because of bacterial resistance, altered drug distribution, patient non-compliance or poor drug availabilty. (Hopkins, R. J. and J. G. Morris, supra). Treatment with antibiotics combined with bismuth are part of the standard regime used to treat H. pylori infection. (Malfertheiner, P. and J. E. Dominguez-Munoz (1993) Clinical Therapeutics 15 Supp. B: 37-48). Recently, combinations of a proton pump inhibitors and a single antibiotic have been shown to ameliorate duodenal ulcer disease. (Malfertheiner, P. and J. E. Dominguez-Munoz supra). However, methods employing antibiotic agents can have the problem of the emergence of bacterial strains which are resistant to these agents. (Hopkins, R. J. and J. G. Morris, supra). These limitations demonstrate that new more effective methods are needed to combat H. pylori infections in vivo. In particular, the design of new vaccines that may prevent infection by this bacterium is highly desirable.

### SUMMARY OF THE INVENTION

This invention relates to novel genes, e.g., genes encoding polypeptides such as bacterial surface proteins, from the organism Helicobacter pylori (H. pylori), and other related genes, their products, and uses thereof. The nucleic acids and peptides of the present invention have utility for diagnostic and therapeutics for H. pylori and other Helicobacter species. They can also be used to detect the presence of H. pylori and other Helicobacter species in a sample; and for use in screening compounds for the ability to interfere with the H. pylori life cycle or to inhibit H. pylori infection. More specifically, this invention features compositions of nucleic acids corresponding to entire coding sequences of H. pylori proteins, including surface or secreted proteins or parts thereof, nucleic acids capable of binding mRNA from H. pylori proteins to block protein translation, and methods for producing H. pylori proteins or parts thereof using peptide synthesis and recombinant DNA techniques. This invention also features antibodies and nucleic acids useful as probes to detect H. pylori infection. In addition, compositions, including vaccine compositions, and methods for the protection or treatment of infection by H. pylori are within the scope of this invention.

### DETAILED DESCRIPTION OF THE DRAWINGS

FIG. 1 is a bar graph that depicts the antibody titer in serum of mice following immunization with specific H. pylori antigens.

FIG. 2 is a bar graph that depicts the antibody titer in mucous of mice following immunization with specific H. pylori antigens.

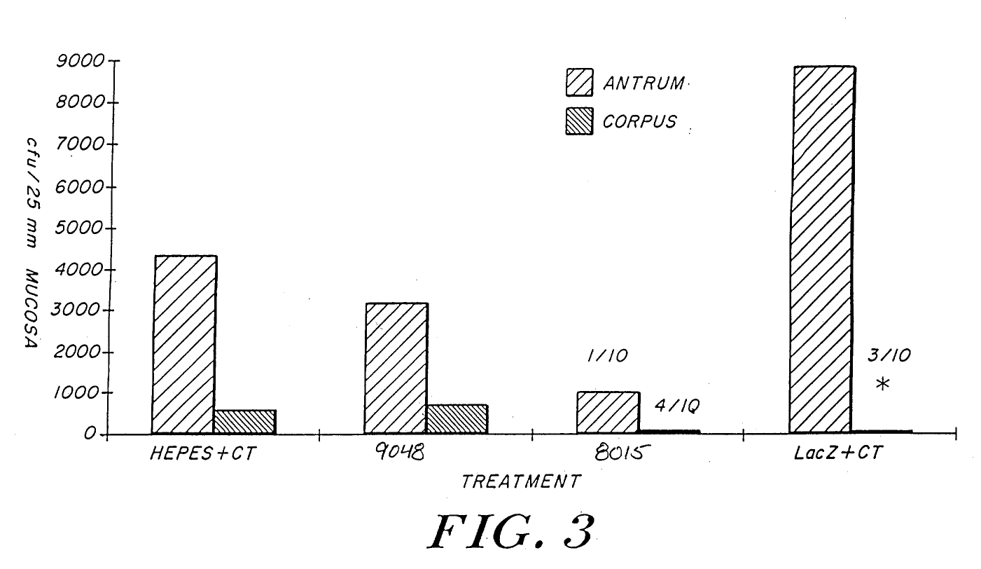

FIG. 3 is a bar graph that depicts therapeutic immunization of H. pylori infected mice with specific antigens dissolved in HEPES buffer.

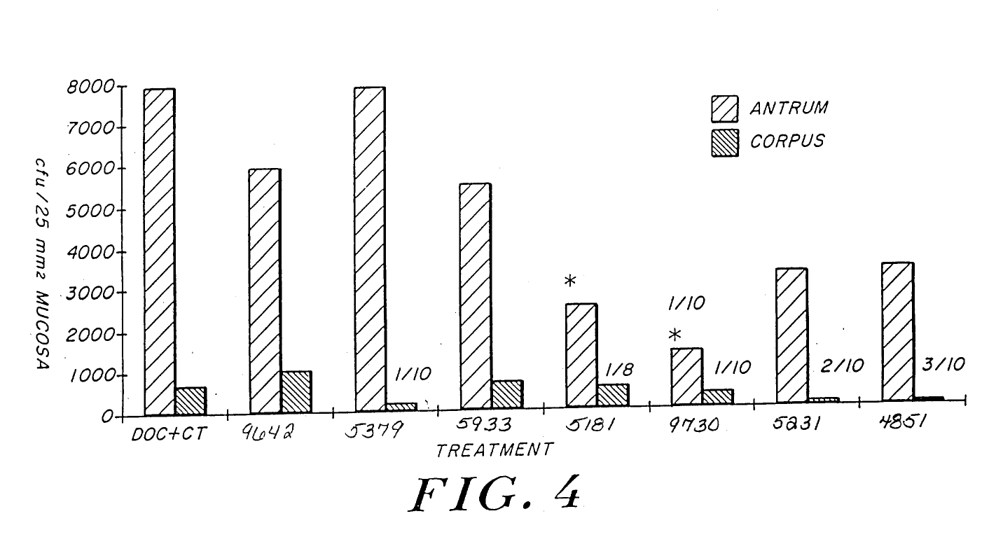

FIG. 4 is a bar graph that depicts therapeutic immunization of H. pylori infected mice with specific antigens dissolved in buffer containing DOC.

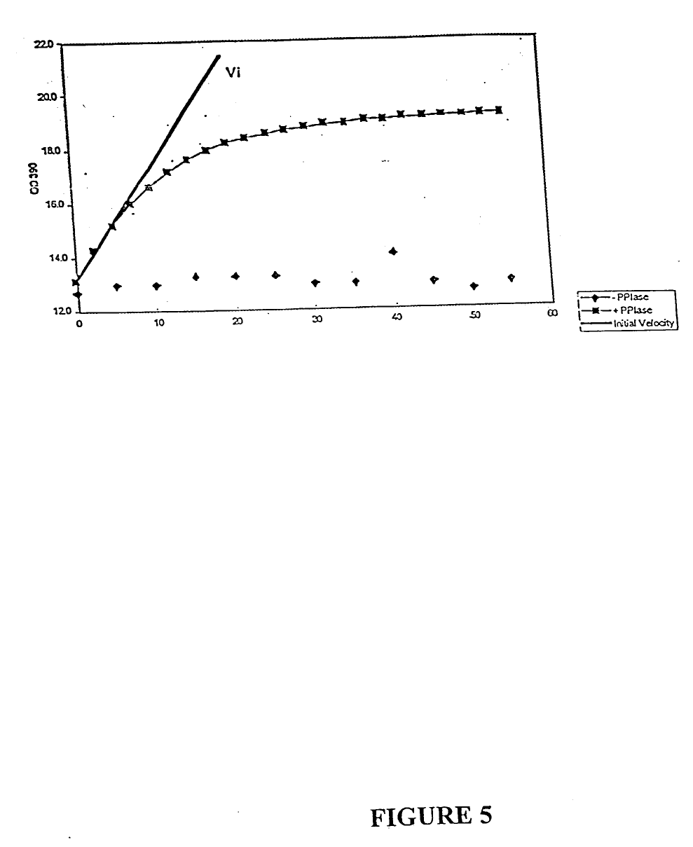

FIG. 5 is a graph depicting the activity of recombinant PPIase.

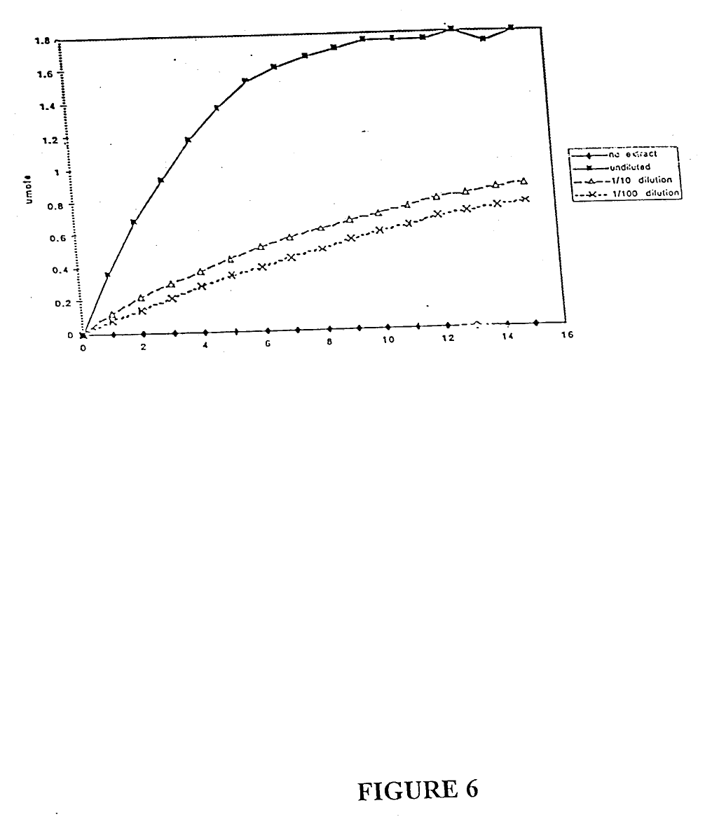

FIG. 6 is a graph depicting PPIase activity in an H. pylori extract.

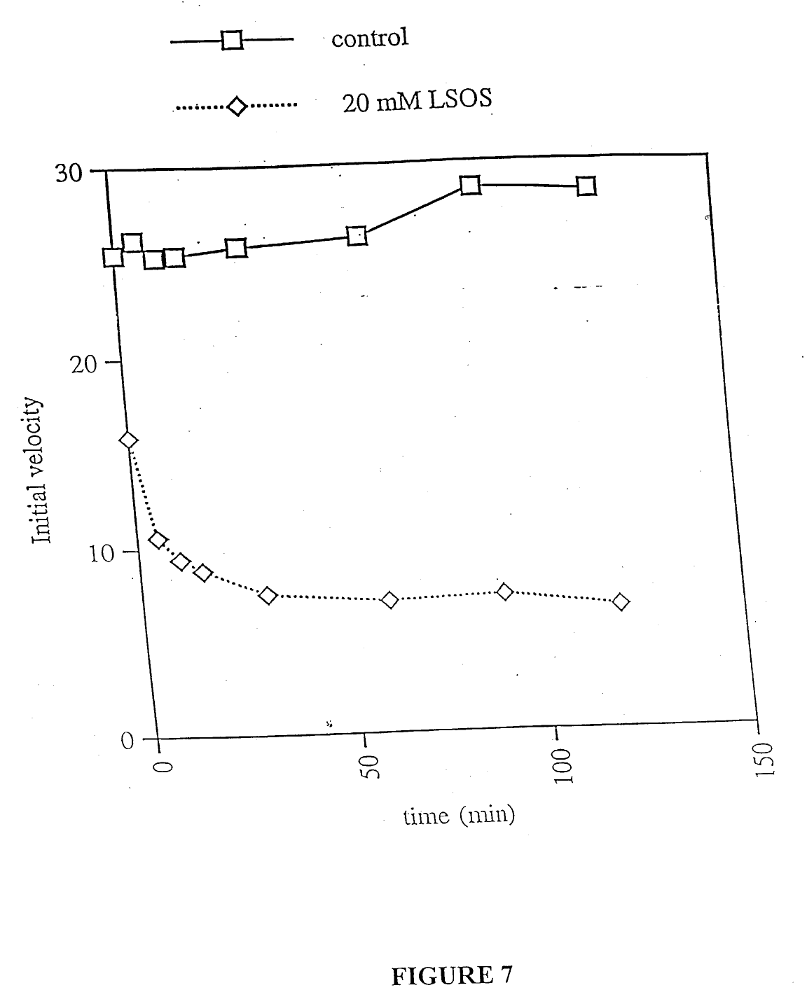

FIG. 7 is a graph depicting inhibition of glutamate racemase activity by L-Serine-O-Sulfate.

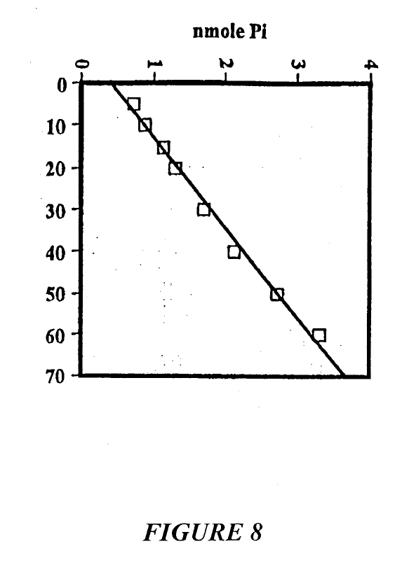

FIG. 8 is a graph depicting the results of an assay of the 3-deoxy-D-manno-2-octulosonic-8-phosphate (KDO-8-P) catalyzed reaction.

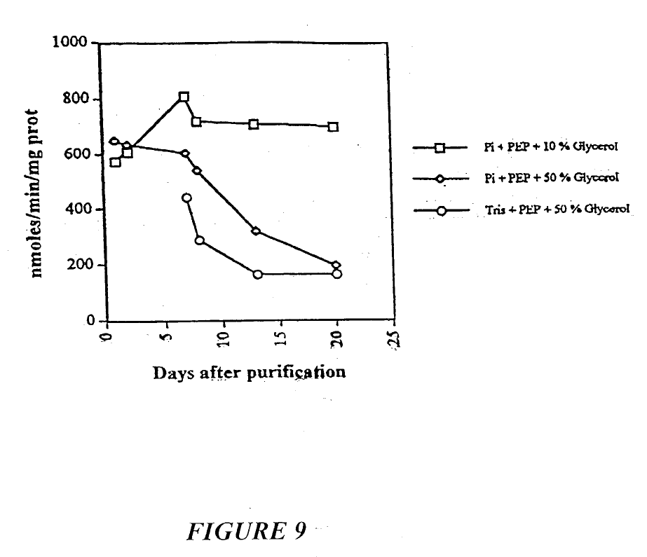

FIG. 9 is a graph depicting the stability of 3-deoxy-D-manno-2-octulosonic-8-phosphate (KDO-8-P) after purification.

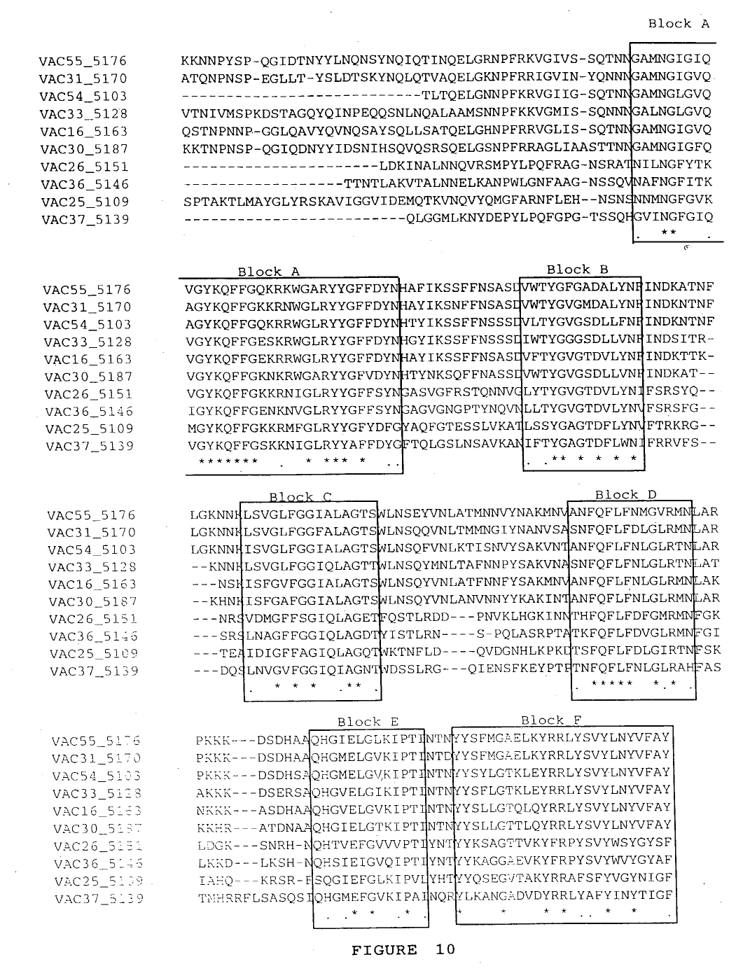

FIG. 10 depicts predicted amphipathic beta-sheet regions at the C-terminus as well as additional amino acid sequence motifs of ten H. pylori outer membrane proteins.

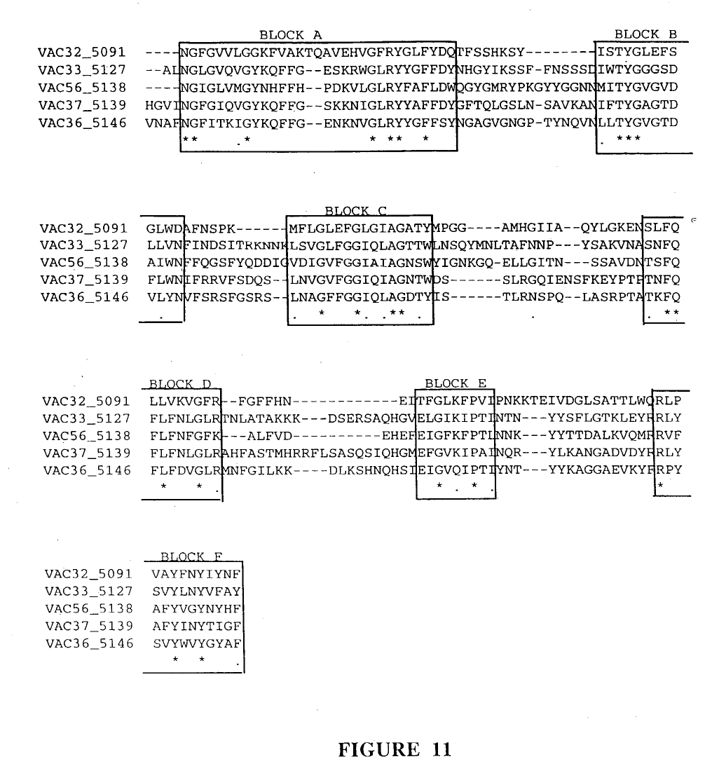

FIG. 11 depicts predicted amphipathic beta-sheet regions at the C-terminus as well as additional amino acid sequence motifs of five H. pylori outer membrane proteins.

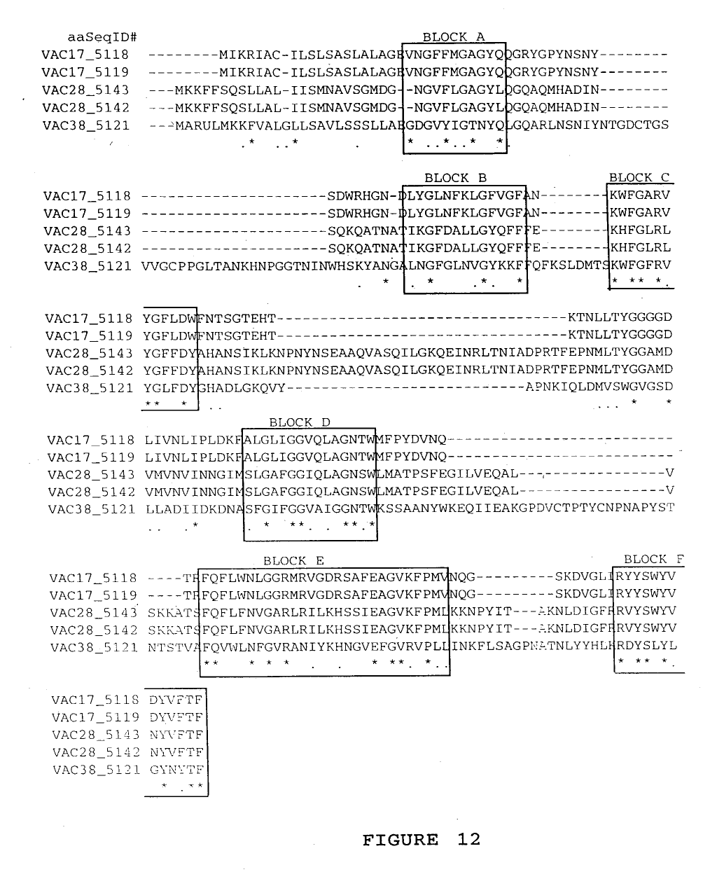

FIG. 12 depicts predicted amphipathic beta-sheet regions at the C-terminus as well as additional amino acid sequence motifs of five H. pylori outer membrane proteins.

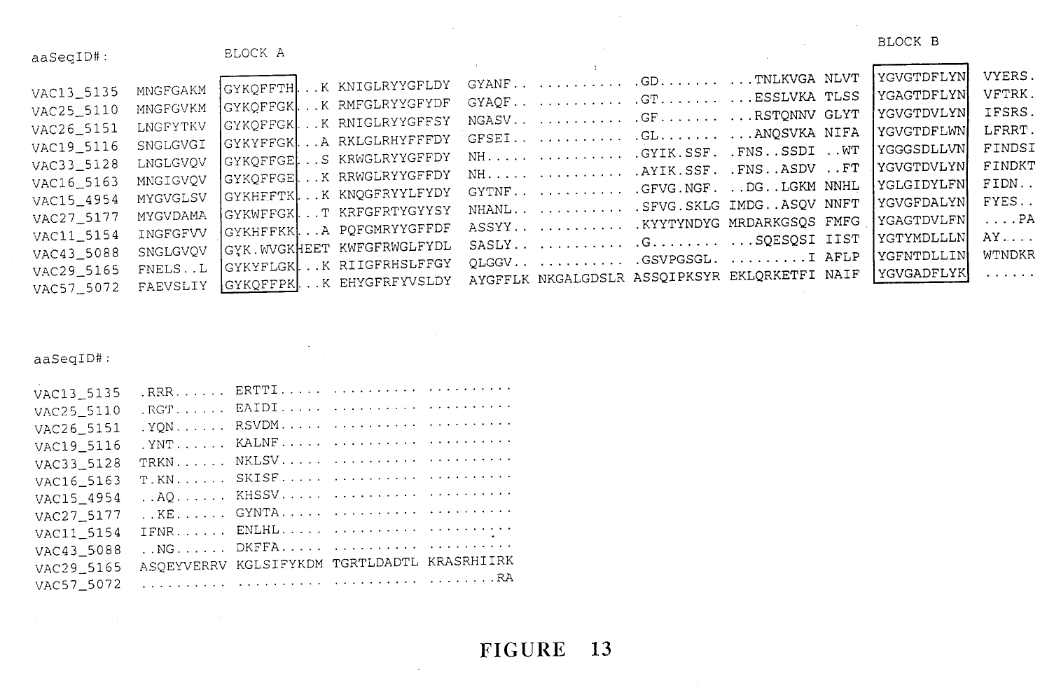

FIG. 13 depicts amino acid sequence motifs of twelve H. pylori outer membranes.

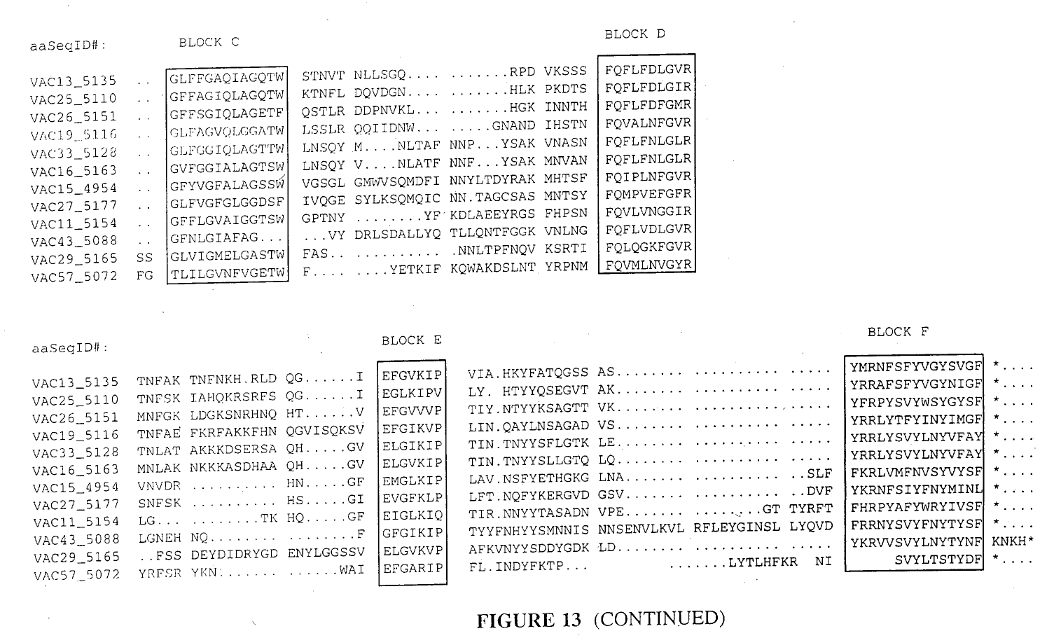

FIG. 14 depicts sequence similarities in the N-terminal portion of six H. pylori proteins.

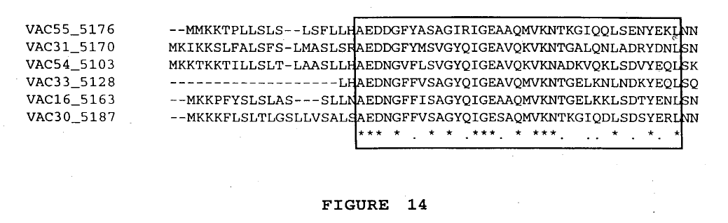

FIG. 15 depicts two members of a family of H. pylori outer membrane proteins which share significant homology across most of their sequences.

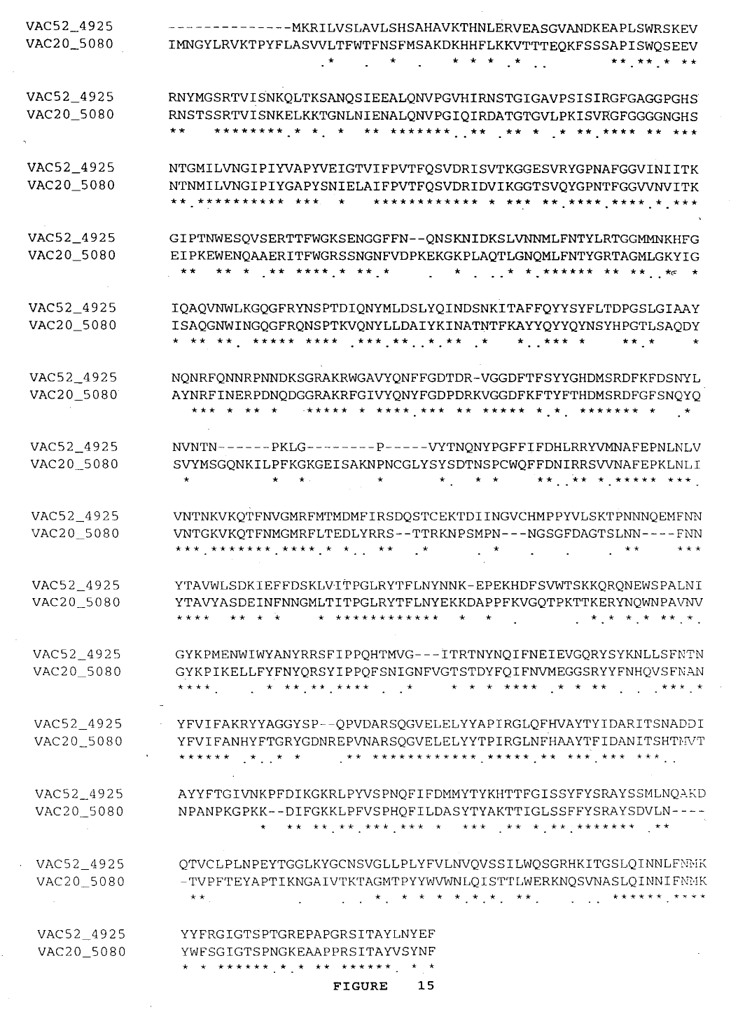

FIG. 16 depicts two members of a family of H. pylori outer membrane proteins which share significant homology across most of their sequences.

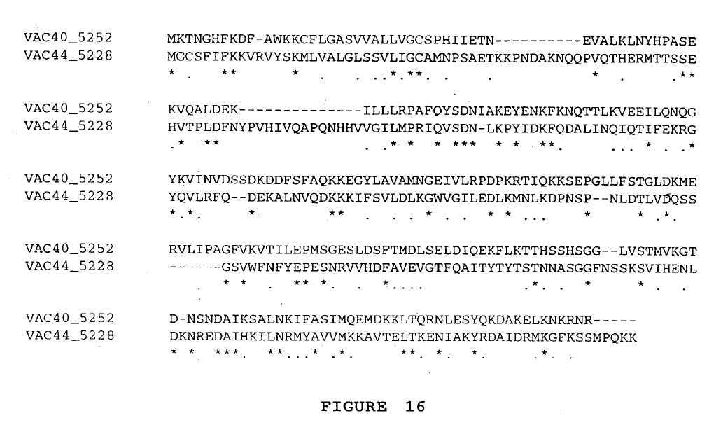

FIG. 17 depicts five members of a family of H. pylori outer membrane proteins which share significant homology across most of their sequences.

### DETAILED DESCRIPTION OF THE INVENTION

In one aspect, the invention features a recombinant or substantially pure preparation of H. pylori polypeptide of SEQ ID NO: 4763. The invention also includes substantially pure nucleic acid encoding an H. pylori polypeptide of SEQ ID NO: 4763, such nucleic acid is contained in SEQ ID NO: 1. The H. pylori polypeptide sequences of the invention described herein are contained in the Sequence Listing, and the nucleic acids encoding H. pylori polypeptides of the invention are contained in the Sequence Listing.

In another aspect, the invention features a recombinant or substantially pure preparation of H. pylori polypeptide selected from the group consisting of H. pylori polypeptides of SEQ ID NO: 4763 through SEQ ID NO: 5012. The invention also includes substantially pure nucleic acid encoding an H. pylori polypeptide selected from the group consisting of SEQ ID NO: 4763 through SEQ ID NO: 5012, such nucleic acids are contained in SEQ ID NO: 1 through SEQ ID NO:250.

In another aspect, the invention features a recombinant or substantially pure preparation of H. pylori polypeptide selected from the group consisting of H. pylori polypeptides of SEQ ID NO: 5013 through SEQ ID NO: 5262. The invention also includes substantially pure nucleic acid encoding an H. pylori polypeptide selected from the group consisting of SEQ ID NO: 5013 through SEQ ID NO: 5262, such nucleic acids are contained in SEQ ID NO: 251 through SEQ ID NO:500.

In another aspect, the invention features a recombinant or substantially pure preparation of H. pylori polypeptide selected from the group consisting of H. pylori polypeptides of SEQ ID NO: 5263 through SEQ ID NO: 5512. The invention also includes substantially pure nucleic acid encoding an H. pylori polypeptide selected from the group consisting of SEQ ID NO: 5263 through SEQ ID NO: 5512, such nucleic acids are contained in SEQ ID NO: 501 through SEQ ID NO:750.

In another aspect, the invention features a recombinant or substantially pure preparation of H. pylori polypeptide selected from the group consisting of H. pylori polypeptides of SEQ ID NO: 5513 through SEQ ID NO: 5762. The invention also includes substantially pure nucleic acid encoding an H. pylori polypeptide selected from the group consisting of SEQ ID NO: 5513 through SEQ ID NO: 5762, such nucleic acids are contained in SEQ ID NO: 751 through SEQ ID NO:1000.

In another aspect, the invention features a recombinant or substantially pure preparation of H. pylori polypeptide selected from the group consisting of H. pylori polypeptides of SEQ ID NO: 5763 through SEQ ID NO: 6012. The invention also includes substantially pure nucleic acid encoding an H. pylori polypeptide selected from the group consisting of SEQ ID NO: 5763 through SEQ ID NO: 6012, such nucleic acids are contained in SEQ ID NO: 1001 through SEQ ID NO:1250.

In another aspect, the invention features a recombinant or substantially pure preparation of H. pylori polypeptide selected from the group consisting of H. pylori polypeptides of SEQ ID NO: 6013 through SEQ ID NO: 6262 The invention also includes substantially pure nucleic acid encoding an H. pylori polypeptide selected from the group consisting of SEQ ID NO: 6013 through SEQ ID NO: 6262, such nucleic acids are contained in SEQ ID NO: 1125 through SEQ ID NO:1500.

In another aspect, the invention features a recombinant or substantially pure preparation of H. pylori polypeptide selected from the group consisting of H. pylori polypeptides of SEQ ID NO: 6263 through SEQ ID NO: 6512. The invention also includes substantially pure nucleic acid encoding an H. pylori polypeptide selected from the group consisting of SEQ ID NO: 6263 through SEQ ID NO: 6512, such nucleic acids are contained in SEQ ID NO: 1501 through SEQ ID NO:1750.

In another aspect, the invention features a recombinant or substantially pure preparation of H. pylori polypeptide selected from the group consisting of H. pylori polypeptides of SEQ ID NO: 6513 through SEQ ID NO: 6762. The invention also includes substantially pure nucleic acid encoding an H. pylori polypeptide selected from the group consisting of SEQ ID NO: 6513 through SEQ ID NO: 6762, such nucleic acids are contained in SEQ ID NO: 1751 through SEQ ID NO:2000.

In another aspect, the invention features a recombinant or substantially pure preparation of H. pylori polypeptide selected from the group consisting of H. pylori polypeptides of SEQ ID NO: 6763 through SEQ ID NO: 7012. The invention also includes substantially pure nucleic acid encoding an H. pylori polypeptide selected from the group consisting of SEQ ID NO: 6763 through SEQ ID NO: 7012, such nucleic acids are contained in SEQ ID NO: 2001 through SEQ ID NO:2250.

In another aspect, the invention features a recombinant or substantially pure preparation of H. pylori polypeptide selected from the group consisting of H. pylori polypeptides of SEQ ID NO: 7013 through SEQ ID NO: 7262. The invention also includes substantially pure nucleic acid encoding an H. pylori polypeptide selected from the group consisting of SEQ ID NO: 7013 through SEQ ID NO: 7262, such nucleic acids are contained in SEQ ID NO: 2251 through SEQ ID NO:2500.

In another aspect, the invention features a recombinant or substantially pure preparation of H. pylori polypeptide selected from the group consisting of H. pylori polypeptides of SEQ ID NO: 7263 through SEQ ID NO:7512. The invention also includes substantially pure nucleic acid encoding an H. pylori polypeptide selected from the group consisting of SEQ ID NO: 7263 through SEQ ID NO: 7512, such nucleic acids are contained in SEQ ID NO: 2501 through SEQ ID NO:2750.

In another aspect, the invention features a recombinant or substantially pure preparation of H. pylori polypeptide selected from the group consisting of H. pylori polypeptides of SEQ ID NO:7513 through SEQ ID NO:7762. The invention also includes substantially pure nucleic acid encoding an H. pylori polypeptide selected from the group consisting of SEQ ID NO: 7513 through SEQ ID NO: 7762, such nucleic acids are contained in SEQ ID NO: 2751 through SEQ ID NO:3000.

In another aspect, the invention features a recombinant or substantially pure preparation of H. pylori polypeptide selected from the group consisting of H. pylori polypeptides of SEQ ID NO:7763 through SEQ ID NO:8012. The invention also includes substantially pure nucleic acid encoding an H. pylori polypeptide selected from the group consisting of SEQ ID NO: 7763 through SEQ ID NO: 8012, such nucleic acids are contained in SEQ ID NO: 3001 through SEQ ID NO:3250.

In another aspect, the invention features a recombinant or substantially pure preparation of H. pylori polypeptide selected from the group consisting of H. pylori polypeptides of SEQ ID NO:8013 through SEQ ID NO:8262. The invention also includes substantially pure nucleic acid encoding an H. pylori polypeptide selected from the group consisting of SEQ ID NO:8013 through SEQ ID NO: 8262, such nucleic acids are contained in SEQ ID NO: 3251 through SEQ ID NO:3500.

In another aspect, the invention features a recombinant or substantially pure preparation of H. pylori polypeptide selected from the group consisting of H. pylori polypeptides of SEQ ID NO:8263 through SEQ ID NO:8512. The invention also includes substantially pure nucleic acid encoding an H. pylori polypeptide selected from the group consisting of SEQ ID NO:8263 through SEQ ID NO: 8512, such nucleic acids are contained in SEQ ID NO: 3501 through SEQ ID NO:3750.

In another aspect, the invention features a recombinant or substantially pure preparation of H. pylori polypeptide selected from the group consisting of H. pylori polypeptides of SEQ ID NO:8513 through SEQ ID NO:8762 The invention also includes substantially pure nucleic acid encoding an H. pylori polypeptide selected from the group consisting of SEQ ID NO:8513 through SEQ ID NO: 8762, such nucleic acids are contained in SEQ ID NO: 3751 through SEQ ID NO:4000.

In another aspect, the invention features a recombinant or substantially pure preparation of H. pylori polypeptide selected from the group consisting of H. pylori polypeptides of SEQ ID NO:8763 through SEQ ID NO:9012. The invention also includes substantially pure nucleic acid encoding an H. pylori polypeptide selected from the group consisting of SEQ ID NO:8763 through SEQ ID NO: 9012, such nucleic acids are contained in SEQ ID NO: 4001 through SEQ ID NO:4250.

In another aspect, the invention features a recombinant or substantially pure preparation of H. pylori polypeptide selected from the group consisting of H. pylori polypeptides of SEQ ID NO:9013 through SEQ ID NO:9262 The invention also includes substantially pure nucleic acid encoding an H. pylori polypeptide selected from the group consisting of SEQ ID NO:9013 through SEQ ID NO: 9262, such nucleic acids are contained in SEQ ID NO: 4251 through SEQ ID NO:4500.

In another aspect, the invention features a recombinant or substantially pure preparation of H. pylori polypeptide selected from the group consisting of H. pylori polypeptides of SEQ ID NO:9263 through SEQ ID NO:9512. The invention also includes substantially pure nucleic acid encoding an H. pylori polypeptide selected from the group consisting of SEQ ID NO:9263 through SEQ ID NO: 9512, such nucleic acids are contained in SEQ ID NO: 4501 through SEQ ID NO:4750.

In another aspect, the invention features a recombinant or substantially pure preparation of H. pylori polypeptide selected from the group consisting of H. pylori polypeptides of SEQ ID NO:9513 through SEQ ID NO:9524. The invention also includes substantially pure nucleic acid encoding an H. pylori polypeptide selected from the group consisting of SEQ ID NO:9513 through SEQ ID NO: 9524, such nucleic acids are contained in SEQ ID NO: 4751 through SEQ ID NO:4762.

In another aspect, the invention features a recombinant or substantially pure preparation of H. pylori polypeptide selected from the group consisting of H. pylori polypeptides of SEQ ID NO:9637 through SEQ ID NO: 9798. The invention also includes substantially pure nucleic acid encoding an H. pylori polypeptide selected from the group consisting of SEQ ID NO: 9637 through SEQ ID NO: 9798, such nucleic acids are contained in SEQ ID NO: 9525 through SEQ ID 9636.

In another aspect, the invention features a recombinant or substantially pure preparation of an H. pylori polypeptide selected from the group consisting of H. pylori polypeptides as set forth in the Sequence Listing. The invention also includes substantially pure nucleic acid encoding an H. pylori polypeptide selected from the group consisting of H. pylori polypeptides as set forth in the Sequence Listing. It should be understood that this invention encompasses each of the H. pylori polypeptides and nucleic acids encoding such polypeptides as identified in the Sequence Listing by a given sequence identification number. For example, a representative H. pylori polypeptide is contained in SEQ ID NO: 4763. Therefore, this invention encompasses a recombinant or substantially pure preparation of an H. pylori polypeptide of SEQ ID NO: 4763. The invention also includes substantially pure nucleic acid encoding an H. pylori polypeptide of SEQ ID NO: 4763.

In another aspect, the invention pertains to any individual H. pylori polypeptide member or nucleic acid encoding such member from the above-identified groups of H. pylori polypeptides (e.g., SEQ ID NO: 4763-SEQ ID NO: 9524 and SEQ ID NO: 9637-SEQ ID NO: 9748) or nucleic acids (e.g., SEQ ID NO: 1-SEQ ID NO: 4762 and SEQ ID NO: 9525-SEQ ID NO: 9636), as well as any subgroups from within the above-identified groups. Furthermore, the subgroups can preferably consist of 1, 3, 5, 10, 15, 20, 30, 40, 50, 75, 100, 125, 150, 175, 200 or 225 members of any of the groups identified above, as well as, any combinations thereof. For example, the group consisting of H. pylori polypeptides SEQ ID NO: 4763 through SEQ ID NO: 5012 can be divided into one or more subgroups as follows: SEQ ID NO: 4763-SEQ ID NO: 4800; SEQ ID NO: 4801-SEQ ID NO: 4860; SEQ ID NO: 4861-SEQ ID NO: 4950; SEQ ID NO: 4951-SEQ ID NO: 5012; or any combinations thereof.

Particularly preferred H. pylori polypeptide or fragments thereof comprises a purified H. pylori murI polypeptide or a fragment thereof, wherein the polypeptide comprises the amino acid sequence of SEQ ID NO: 7635. The invention also includes an isolated nucleic acid encoding a H. pylori murI polypeptide, which nucleic acid comprises the nucleotide sequence of SEQ ID NO: 2873.

Particularly preferred H. pylori polypeptide or fragments thereof comprises a purified H. pylori murC polypeptide or a fragment thereof, wherein the polypeptide comprises the amino acid sequence of SEQ ID NO: 7607. The invention also includes an isolated nucleic acid encoding a H. pylori murC polypeptide, which nucleic acid comprises the nucleotide sequence of SEQ ID NO: 2845.

In another aspect, the invention features an isolated nucleic acid having a nucleotide sequence encoding an H. pylori polypeptide at least about 60% homologous to an amino acid sequence selected from the group consisting of SEQ ID NO: 4763-SEQ ID NO: 9524 and SEQ ID NO: 9637-SEQ ID NO: 9748. In a preferred embodiment, the isolated nucleic acid includes a nucleotide sequence selected from the group consisting of SEQ ID NO: 1-SEQ ID NO: 4762 and SEQ ID NO: 9525-SEQ ID NO: 9636, or a complement thereof.

In another aspect, the invention features an isolated nucleic acid having a nucleotide sequence encoding an H. pylori polypeptide selected from the group consisting of SEQ ID NO: 4763-SEQ ID NO: 9524 and SEQ ID NO: 9637-SEQ ID NO: 9748.

In another aspect, the invention features an isolated nucleic acid which encodes an H. pylori polypeptide, having a nucleotide sequence at least about 60% homologous to a nucleotide sequence selected from the group consisting of SEQ ID NO: 1-SEQ ID NO: 4762 and SEQ ID NO: 9525-SEQ ID NO: 9636, or a complement thereof.

In another aspect, the invention features an isolated nucleic acid molecule encoding an H. pylori polypeptide, having a nucleotide sequence which hybridizes under stringent hybridization conditions to a nucleic acid molecule having the nucleotide sequence selected from the group consisting of SEQ ID NO: 1-SEQ ID NO: 4762 and SEQ ID NO: 9525-SEQ ID NO: 9636, or a complement thereof.

In another aspect, the invention features an isolated nucleic acid having a nucleotide sequence of at least 8 nucleotides in length, wherein the sequence hybridizes under stringent hybridization conditions to a nucleic acid having a nucleotide sequence selected from the group consisting of SEQ ID NO: 1-SEQ ID NO: 4762 and SEQ ID NO: 9525-SEQ ID NO: 9636, or a complement thereof.

Particularly preferred is an isolated nucleic acid having a nucleotide sequence encoding an H. pylori cell envelope polypeptide or a fragment thereof, the nucleic acid selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 11; SEQ ID NO: 112; SEQ ID NO: 113; SEQ ID NO: 114; SEQ ID NO: 115; SEQ ID NO: 116; SEQ ID NO: 117; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; SEQ ID NO: 186; SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; SEQ ID NO: 211; SEQ ID NO: 212; SEQ ID NO: 213; SEQ ID NO: 214; SEQ ID NO: 215; SEQ ID NO: 216; SEQ ID NO: 217; SEQ ID NO: 218; SEQ ID NO: 219; SEQ ID NO: 220; SEQ ID NO: 221; SEQ ID NO: 222; SEQ ID NO: 223; SEQ ID NO: 224; SEQ ID NO: 225; SEQ ID NO: 226; SEQ ID NO: 227; SEQ ID NO: 228; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 236; SEQ ID NO: 237; SEQ ID NO: 238; SEQ ID NO: 239; SEQ ID NO: 240; SEQ ID NO: 241; SEQ ID NO: 242; SEQ ID NO: 243; SEQ ID NO: 244; SEQ ID NO: 245; SEQ ID NO: 246; SEQ ID NO: 247; SEQ ID NO: 248; SEQ ID NO: 249; SEQ ID NO: 250; SEQ ID NO: 251; SEQ ID NO: 252; SEQ ID NO: 253; SEQ ID NO: 254; SEQ ID NO: 255; SEQ ID NO: 256; SEQ ID NO: 257; SEQ ID NO: 258; SEQ ID NO: 259; SEQ ID NO: 260; SEQ ID NO: 261; SEQ ID NO: 262; SEQ ID NO: 263; SEQ ID NO: 264; SEQ ID NO: 265; SEQ ID NO: 266; SEQ ID NO: 267; SEQ ID NO: 268; SEQ ID NO: 269; SEQ ID NO: 270; SEQ ID NO: 271; SEQ ID NO: 272; SEQ ID NO: 273; SEQ ID NO: 274; SEQ ID NO: 275; SEQ ID NO: 276; SEQ ID NO: 277; SEQ ID NO: 278; SEQ ID NO: 279; SEQ ID NO: 280; SEQ ID NO: 281; SEQ ID NO: 282; SEQ ID NO: 283; SEQ ID NO: 284; SEQ ID NO: 285; SEQ ID NO: 286; SEQ ID NO: 287; SEQ ID NO: 288; SEQ ID NO: 289; SEQ ID NO: 290; SEQ ID NO: 291; SEQ ID NO: 292; SEQ ID NO: 293; SEQ ID NO: 294; SEQ ID NO: 295; SEQ ID NO: 296; SEQ ID NO: 297; SEQ ID NO: 298; SEQ ID NO: 299; SEQ ID NO: 300; SEQ ID NO: 301; SEQ ID NO: 302; SEQ ID NO: 303; SEQ ID NO: 304; SEQ ID NO: 305; SEQ ID NO: 306; SEQ ID NO: 307; SEQ ID NO: 308; SEQ ID NO: 309; SEQ ID NO: 310; SEQ ID NO: 311; SEQ ID NO: 312; SEQ ID NO: 313; SEQ ID NO: 314; SEQ ID NO: 315; SEQ ID NO: 316; SEQ ID NO: 317; SEQ ID NO: 318; SEQ ID NO: 319; SEQ ID NO: 320; SEQ ID NO: 321; SEQ ID NO: 322; SEQ ID NO: 323; SEQ ID NO: 324; SEQ ID NO: 325; SEQ ID NO: 326; SEQ ID NO: 327; SEQ ID NO: 328; SEQ ID NO: 329; SEQ ID NO: 330; SEQ ID NO: 331; SEQ ID NO: 332; SEQ ID NO: 333; SEQ ID NO: 334; SEQ ID NO: 335; SEQ ID NO: 336; SEQ ID NO: 337; SEQ ID NO: 338; SEQ ID NO: 339; SEQ ID NO: 340; SEQ ID NO: 341; SEQ ID NO: 342; SEQ ID NO: 343; SEQ ID NO: 344; SEQ ID NO: 345; SEQ ID NO: 346; SEQ ID NO: 347; SEQ ID NO: 348; SEQ ID NO: 349; SEQ ID NO: 350; SEQ ID NO: 351; SEQ ID NO: 352; SEQ ID NO: 353; SEQ ID NO: 354; SEQ ID NO: 355; SEQ ID NO: 356; SEQ ID NO: 357; SEQ ID NO: 358; SEQ ID NO: 359; SEQ ID NO: 360; SEQ ID NO: 361; SEQ ID NO: 362; SEQ ID NO: 363; SEQ ID NO: 364; SEQ ID NO: 365; SEQ ID NO: 366; SEQ ID NO: 367; SEQ ID NO: 368; SEQ ID NO: 369; SEQ ID NO: 370; SEQ ID NO: 371; SEQ ID NO: 372; SEQ ID NO: 373; SEQ ID NO: 374; SEQ ID NO: 375; SEQ ID NO: 376; SEQ ID NO: 377; SEQ ID NO: 378; SEQ ID NO: 379; SEQ ID NO: 380; SEQ ID NO: 381; SEQ ID NO: 382; SEQ ID NO: 383; SEQ ID NO: 384; SEQ ID NO: 385; SEQ ID NO: 386; SEQ ID NO: 387; SEQ ID NO: 388; SEQ ID NO: 389; SEQ ID NO: 390; SEQ ID NO: 391; SEQ ID NO: 392; SEQ ID NO: 393; SEQ ID NO: 394; SEQ ID NO: 395; SEQ ID NO: 396; SEQ ID NO: 397; SEQ ID NO: 398; SEQ ID NO: 399; SEQ ID NO: 400; SEQ ID NO: 401; SEQ ID NO: 402; SEQ ID NO: 403; SEQ ID NO: 404; SEQ ID NO: 405; SEQ ID NO: 406; SEQ ID NO: 407; SEQ ID NO: 408; SEQ ID NO: 409; SEQ ID NO: 410; SEQ ID NO: 411; SEQ ID NO: 412; SEQ ID NO: 413; SEQ ID NO: 414; SEQ ID NO: 415; SEQ ID NO: 416; SEQ ID NO: 417; SEQ ID NO: 418; SEQ ID NO: 419; SEQ ID NO: 420; SEQ ID NO: 421; SEQ ID NO: 422; SEQ ID NO: 423; SEQ ID NO: 424; SEQ ID NO: 425; SEQ ID NO: 426; SEQ ID NO: 427; SEQ ID NO: 428; SEQ ID NO: 429; SEQ ID NO: 430; SEQ ID NO: 431; SEQ ID NO: 432; SEQ ID NO: 433; SEQ ID NO: 434; SEQ ID NO: 435; SEQ ID NO: 436; SEQ ID NO: 437; SEQ ID NO: 438; SEQ ID NO: 439; SEQ ID NO: 440; SEQ ID NO: 441; SEQ ID NO: 442; SEQ ID NO: 443; SEQ ID NO: 444; SEQ ID NO: 445; SEQ ID NO: 446; SEQ ID NO: 447; SEQ ID NO: 448; SEQ ID NO: 449; SEQ ID NO: 450; SEQ ID NO: 451; SEQ ID NO: 452; SEQ ID NO: 453; SEQ ID NO: 454; SEQ ID NO: 455; SEQ ID NO: 456; SEQ ID NO: 457; SEQ ID NO: 458; SEQ ID NO: 459; SEQ ID NO: 460; SEQ ID NO: 461; SEQ ID NO: 462; SEQ ID NO: 463; SEQ ID NO: 464; SEQ ID NO: 465; SEQ ID NO: 466; SEQ ID NO: 467; SEQ ID NO: 468; SEQ ID NO: 469; SEQ ID NO: 470; SEQ ID NO: 471; SEQ ID NO: 472; SEQ ID NO: 473; SEQ ID NO: 474; SEQ ID NO: 475; SEQ ID NO: 476; SEQ ID NO: 477; SEQ ID NO: 478; SEQ ID NO: 479; SEQ ID NO: 480; SEQ ID NO: 481; SEQ ID NO: 482; SEQ ID NO: 483; SEQ ID NO: 484; SEQ ID NO: 485; SEQ ID NO: 486; SEQ ID NO: 487; SEQ ID NO: 488; SEQ ID NO: 489; SEQ ID NO: 490; SEQ ID NO: 491; SEQ ID NO: 492; SEQ ID NO: 493; SEQ ID NO: 494; SEQ ID NO: 495; SEQ ID NO: 496; SEQ ID NO: 497; SEQ ID NO: 498; SEQ ID NO: 499; SEQ ID NO: 500; SEQ ID NO: 501; SEQ ID NO: 502; SEQ ID NO: 503; SEQ ID NO: 504; SEQ ID NO: 505; SEQ ID NO: 506; SEQ ID NO: 507; SEQ ID NO: 508; SEQ ID NO: 509; SEQ ID NO: 510; SEQ ID NO: 511; SEQ ID NO: 512; SEQ ID NO: 513; SEQ ID NO: 514; SEQ ID NO: 515; SEQ ID NO: 516; SEQ ID NO: 517; SEQ ID NO: 518; SEQ ID NO: 519; SEQ ID NO: 520; SEQ ID NO: 521; SEQ ID NO: 522; SEQ ID NO: 523; SEQ ID NO: 524; SEQ ID NO: 525; SEQ ID NO: 526; SEQ ID NO: 527; SEQ ID NO: 528; SEQ ID NO: 529; SEQ ID NO: 530; SEQ ID NO: 531; SEQ ID NO: 532; SEQ ID NO: 533; SEQ ID NO: 534; SEQ ID NO: 535; SEQ ID NO: 536; SEQ ID NO: 537; SEQ ID NO: 538; SEQ ID NO: 539; SEQ ID NO: 540; SEQ ID NO: 541; SEQ ID NO: 542; SEQ ID NO: 543; SEQ ID NO: 544; SEQ ID NO: 545; SEQ ID NO: 546; SEQ ID NO: 547; SEQ ID NO: 548; SEQ ID NO: 549; SEQ ID NO: 550; SEQ ID NO: 551; SEQ ID NO: 552; SEQ ID NO: 553; SEQ ID NO: 554; SEQ ID NO: 555; SEQ ID NO: 556; SEQ ID NO: 557; SEQ ID NO: 558; SEQ ID NO: 559; SEQ ID NO: 560; SEQ ID NO: 561; SEQ ID NO: 562; SEQ ID NO: 563; SEQ ID NO: 564; SEQ ID NO: 565; SEQ ID NO: 566; SEQ ID NO: 567; SEQ ID NO: 568; SEQ ID NO: 569; SEQ ID NO: 570; SEQ ID NO: 571; SEQ ID NO: 572; SEQ ID NO: 573; SEQ ID NO: 574; SEQ ID NO: 575; SEQ ID NO: 576; SEQ ID NO: 577; SEQ ID NO: 578; SEQ ID NO: 579; SEQ ID NO: 580; SEQ ID NO: 581; SEQ ID NO: 582; SEQ ID NO: 583; SEQ ID NO: 584; SEQ ID NO: 585; SEQ ID NO: 586; SEQ ID NO: 587; SEQ ID NO: 588; SEQ ID NO: 589; SEQ ID NO: 590; SEQ ID NO: 591; SEQ ID NO: 592; SEQ ID NO: 593; SEQ ID NO: 594; SEQ ID NO: 595; SEQ ID NO: 596; SEQ ID NO: 597; SEQ ID NO: 598; SEQ ID NO: 599; SEQ ID NO: 600; SEQ ID NO: 601; SEQ ID NO: 602; SEQ ID NO: 603; SEQ ID NO: 604; SEQ ID NO: 605; SEQ ID NO: 606; SEQ ID NO: 607; SEQ ID NO: 608; SEQ ID NO: 609; SEQ ID NO: 610; SEQ ID NO: 611; SEQ ID NO: 612; SEQ ID NO: 613; SEQ ID NO: 614; SEQ ID NO: 615; SEQ ID NO: 616; SEQ ID NO: 617; SEQ ID NO: 618; SEQ ID NO: 619; SEQ ID NO: 620; SEQ ID NO: 621; SEQ ID NO: 622; SEQ ID NO: 623; SEQ ID NO: 624; SEQ ID NO: 625; SEQ ID NO: 626; SEQ ID NO: 627; SEQ ID NO: 628; SEQ ID NO: 629; SEQ ID NO: 630; SEQ ID NO: 631; SEQ ID NO: 632; SEQ ID NO: 633; SEQ ID NO: 634; SEQ ID NO: 635; SEQ ID NO: 636; SEQ ID NO: 637; SEQ ID NO: 638; SEQ ID NO: 639; SEQ ID NO: 640; SEQ ID NO: 641; SEQ ID NO: 642; SEQ ID NO: 643; SEQ ID NO: 644; SEQ ID NO: 645; SEQ ID NO: 646; SEQ ID NO: 647; SEQ ID NO: 648; SEQ ID NO: 649; SEQ ID NO: 650; SEQ ID NO: 651; SEQ ID NO: 652; SEQ ID NO: 653; SEQ ID NO: 654; SEQ ID NO: 655; SEQ ID NO: 656; SEQ ID NO: 657; SEQ ID NO: 658; SEQ ID NO: 659; SEQ ID NO: 660; SEQ ID NO: 661; SEQ ID NO: 662; SEQ ID NO: 663; SEQ ID NO: 664; SEQ ID NO: 665; SEQ ID NO: 666; SEQ ID NO: 667; SEQ ID NO: 668; SEQ ID NO: 669; SEQ ID NO: 670; SEQ ID NO: 671; SEQ ID NO: 672; SEQ ID NO: 673; SEQ ID NO: 674; SEQ ID NO: 675; SEQ ID NO: 676; SEQ ID NO: 677; SEQ ID NO: 678; SEQ ID NO: 679; SEQ ID NO: 680; SEQ ID NO: 681; SEQ ID NO: 682; SEQ ID NO: 683; SEQ ID NO: 684; SEQ ID NO: 685; SEQ ID NO: 686; SEQ ID NO: 687; SEQ ID NO: 688; SEQ ID NO: 689; SEQ ID NO: 690; SEQ ID NO: 691; SEQ ID NO: 692; SEQ ID NO: 693; SEQ ID NO: 694; SEQ ID NO: 695; SEQ ID NO: 696; SEQ ID NO: 697; SEQ ID NO: 698; SEQ ID NO: 699; SEQ ID NO: 700; SEQ ID NO: 701; SEQ ID NO: 702; SEQ ID NO: 703; SEQ ID NO: 704; SEQ ID NO: 705; SEQ ID NO: 706; SEQ ID NO: 707; SEQ ID NO: 708; SEQ ID NO: 709; SEQ ID NO: 710; SEQ ID NO: 711; SEQ ID NO: 712; SEQ ID NO: 713; SEQ ID NO: 714; SEQ ID NO: 715; SEQ ID NO: 716; SEQ ID NO: 717; SEQ ID NO: 718; SEQ ID NO: 719; SEQ ID NO: 720; SEQ ID NO: 721; SEQ ID NO: 722; SEQ ID NO: 723; SEQ ID NO: 724; SEQ ID NO: 725; SEQ ID NO: 726; SEQ ID NO: 727; SEQ ID NO: 728; SEQ ID NO: 729; SEQ ID NO: 730; SEQ ID NO: 731; SEQ ID NO: 732; SEQ ID NO: 733; SEQ ID NO: 734; SEQ ID NO: 735; SEQ ID NO: 736; SEQ ID NO: 737; SEQ ID NO: 738; SEQ ID NO: 739; SEQ ID NO: 740; SEQ ID NO: 741; SEQ ID NO: 742; SEQ ID NO: 743; SEQ ID NO: 744; SEQ ID NO: 745; SEQ ID NO: 746; SEQ ID NO: 747; SEQ ID NO: 748; SEQ ID NO: 749; SEQ ID NO: 750; SEQ ID NO: 751; SEQ ID NO: 752; SEQ ID NO: 753; SEQ ID NO: 754; SEQ ID NO: 755; SEQ ID NO: 756; SEQ ID NO: 757; SEQ ID NO: 758; SEQ ID NO: 759; SEQ ID NO: 760; SEQ ID NO: 761; SEQ ID NO: 762; SEQ ID NO: 763; SEQ ID NO: 764; SEQ ID NO: 765; SEQ ID NO: 766; SEQ ID NO: 767; SEQ ID NO: 768; SEQ ID NO: 769; SEQ ID NO: 770; SEQ ID NO: 771; SEQ ID NO: 772; SEQ ID NO: 773; SEQ ID NO: 774; SEQ ID NO: 775; SEQ ID NO: 776; SEQ ID NO: 777; SEQ ID NO: 778; SEQ ID NO: 779; SEQ ID NO: 780; SEQ ID NO: 781; SEQ ID NO: 782; SEQ ID NO: 783; SEQ ID NO: 784; SEQ ID NO: 785; SEQ ID NO: 786; SEQ ID NO: 787; SEQ ID NO: 788; SEQ ID NO: 789; SEQ ID NO: 790; SEQ ID NO: 791; SEQ ID NO: 792; SEQ ID NO: 793; SEQ ID NO: 794; SEQ ID NO: 795; SEQ ID NO: 796; SEQ ID NO: 797; SEQ ID NO: 798; SEQ ID NO: 799; SEQ ID NO: 800; SEQ ID NO: 801; SEQ ID NO: 802; SEQ ID NO: 803; SEQ ID NO: 804; SEQ ID NO: 805; SEQ ID NO: 806; SEQ ID NO: 807; SEQ ID NO: 808; SEQ ID NO: 809; SEQ ID NO: 810; SEQ ID NO: 811; SEQ ID NO: 812; SEQ ID NO: 813; SEQ ID NO: 814; SEQ ID NO: 815; SEQ ID NO: 816; SEQ ID NO: 817; SEQ ID NO: 818; SEQ ID NO: 819; SEQ ID NO: 820; SEQ ID NO: 821; SEQ ID NO: 822; SEQ ID NO: 823; SEQ ID NO: 824; SEQ ID NO: 825; SEQ ID NO: 826; SEQ ID NO: 827; SEQ ID NO: 828; SEQ ID NO: 829; SEQ ID NO: 830; SEQ ID NO: 831; SEQ ID NO: 832; SEQ ID NO: 833; SEQ ID NO: 834; SEQ ID NO: 835; SEQ ID NO: 836; SEQ ID NO: 837; SEQ ID NO: 838; SEQ ID NO: 839; SEQ ID NO: 840; SEQ ID NO: 841; SEQ ID NO: 842; SEQ ID NO: 843; SEQ ID NO: 844; SEQ ID NO: 845; SEQ ID NO: 846; SEQ ID NO: 847; SEQ ID NO: 848; SEQ ID NO: 849; SEQ ID NO: 850; SEQ ID NO: 851; SEQ ID NO: 852; SEQ ID NO: 853; SEQ ID NO: 854; SEQ ID NO: 855; SEQ ID NO: 856; SEQ ID NO: 857; SEQ ID NO: 858; SEQ ID NO: 859; SEQ ID NO: 860; SEQ ID NO: 861; SEQ ID NO: 862; SEQ ID NO: 863; SEQ ID NO: 864; SEQ ID NO: 865; SEQ ID NO: 866; SEQ ID NO: 867; SEQ ID NO: 868; SEQ ID NO: 869; SEQ ID NO: 870; SEQ ID NO: 871; SEQ ID NO: 872; SEQ ID NO: 873; SEQ ID NO: 874; SEQ ID NO: 875; SEQ ID NO: 876; SEQ ID NO: 877; SEQ ID NO: 878; SEQ ID NO: 879; SEQ ID NO: 880; SEQ ID NO: 881; SEQ ID NO: 882; SEQ ID NO: 883; SEQ ID NO: 884; SEQ ID NO: 885; SEQ ID NO: 886; SEQ ID NO: 887; SEQ ID NO: 888; SEQ ID NO: 889; SEQ ID NO: 890; SEQ ID NO: 891; SEQ ID NO: 892; SEQ ID NO: 893; SEQ ID NO: 894; SEQ ID NO: 895; SEQ ID NO: 896; SEQ ID NO: 897; SEQ ID NO: 898; SEQ ID NO: 899; SEQ ID NO: 900; SEQ ID NO: 901; SEQ ID NO: 902; SEQ ID NO: 903; SEQ ID NO: 904; SEQ ID NO: 905; SEQ ID NO: 906; SEQ ID NO: 907; SEQ ID NO: 908; SEQ ID NO: 909; SEQ ID NO: 910; SEQ ID NO: 911; SEQ ID NO: 912; SEQ ID NO: 913; SEQ ID NO: 914; SEQ ID NO: 915; SEQ ID NO: 916; SEQ ID NO: 917; SEQ ID NO: 918; SEQ ID NO: 919; SEQ ID NO: 920; SEQ ID NO: 921; SEQ ID NO: 922; SEQ ID NO: 923; SEQ ID NO: 924; SEQ ID NO: 925; SEQ ID NO: 926; SEQ ID NO: 927; SEQ ID NO: 928; SEQ ID NO: 929; SEQ ID NO: 930; SEQ ID NO: 931; SEQ ID NO: 932; SEQ ID NO: 933; SEQ ID NO: 934; SEQ ID NO: 935; SEQ ID NO: 936; SEQ ID NO: 937; SEQ ID NO: 938; SEQ ID NO: 939; SEQ, ID NO: 940; SEQ ID NO: 941; SEQ ID NO: 942; SEQ ID NO: 943; SEQ ID NO: 944; SEQ ID NO: 945; SEQ ID NO: 946; SEQ ID NO: 947; SEQ ID NO: 948; SEQ ID NO: 949; SEQ ID NO: 950; SEQ ID NO: 951; SEQ ID NO: 952; SEQ ID NO: 953; SEQ ID NO: 954; SEQ ID NO: 955; SEQ ID NO: 956; SEQ ID NO: 957; SEQ ID NO: 958; SEQ ID NO: 959; SEQ ID NO: 960; SEQ ID NO: 961; SEQ ID NO: 962; SEQ ID NO: 963; SEQ ID NO: 964; SEQ ID NO: 965; SEQ ID NO: 966; SEQ ID NO: 967; SEQ ID NO: 968; SEQ ID NO: 969; SEQ ID NO: 970; SEQ ID NO: 971; SEQ ID NO: 972; SEQ ID NO: 973; SEQ ID NO: 974; SEQ ID NO: 975; SEQ ID NO: 976; SEQ ID NO: 977; SEQ ID NO: 978; SEQ ID NO: 979; SEQ ID NO: 980; SEQ ID NO: 981; SEQ ID NO: 982; SEQ ID NO: 983; SEQ ID NO: 984; SEQ ID NO: 985; SEQ ID NO: 986; SEQ ID NO: 987; SEQ ID NO: 988; SEQ ID NO: 989; SEQ ID NO: 990; SEQ ID NO: 991; SEQ ID NO: 992; SEQ ID NO: 993; SEQ ID NO: 994; SEQ ID NO: 995; SEQ ID NO: 996; SEQ ID NO: 997; SEQ ID NO: 998; SEQ ID NO: 999; SEQ ID NO: 1000; SEQ ID NO: 1001; SEQ ID NO: 1002; SEQ ID NO: 1003; SEQ ID NO: 1004; SEQ ID NO: 1005; SEQ ID NO: 1006; SEQ ID NO: 1007; SEQ ID NO: 1008; SEQ ID NO: 1009; SEQ ID NO: 1010; SEQ ID NO: 1011; SEQ ID NO: 1012; SEQ ID NO: 1013; SEQ ID NO: 1014; SEQ ID NO: 1015; SEQ ID NO: 1016; SEQ ID NO: 10.7, SEQ ID NO: 1018; SEQ ID NO: 1019; SEQ ID NO: 1020; SEQ ID NO: 1021; SEQ ID NO: 1022; SEQ ID NO: 1023; SEQ ID NO: 1024; SEQ ID NO: 1025; SEQ ID NO: 1026; SEQ ID NO: 1027; SEQ ID NO: 1028; SEQ ID NO: 1029; SEQ ID NO: 1030; SEQ ID NO: 1031; SEQ ID NO: 1032; SEQ ID NO: 1033; SEQ ID NO: 1034; SEQ ID NO: 1035; SEQ ID NO: 1036; SEQ ID NO: 1037; SEQ ID NO: 1038; SEQ ID NO: 1039; SEQ ID NO: 1040; SEQ ID NO: 1041; SEQ ID NO: 1042; SEQ ID NO: 1043; SEQ ID NO: 1044; SEQ ID NO: 1045; SEQ ID NO: 1046; SEQ ID NO: 1047; SEQ ID NO: 1048; SEQ ID NO: 1049; SEQ ID NO: 1050; SEQ ID NO: 1051; SEQ ID NO: 1052; SEQ ID NO: 1053; SEQ ID NO: 1054; SEQ ID NO: 1055; SEQ ID NO: 1056; SEQ ID NO: 1057; SEQ ID NO: 1058; SEQ ID NO: 1059; SEQ ID NO: 1060; SEQ ID NO: 1061; SEQ ID NO: 1062; SEQ ID NO: 1063; SEQ ID NO: 1064; SEQ ID NO: 1065; SEQ ID NO: 1066; SEQ ID NO: 1067; SEQ ID NO: 1068; SEQ ID NO: 1069; SEQ ID NO: 1070; SEQ ID NO: 1071; SEQ ID NO: 1072; SEQ ID NO: 1073; SEQ ID NO: 1074; SEQ ID NO: 1075; SEQ ID NO: 1076; SEQ ID NO: 1077; SEQ ID NO: 1078; SEQ ID NO: 1079; SEQ ID NO: 1080; SEQ ID NO: 1081; SEQ ID NO: 1082; SEQ ID NO: 1083; SEQ ID NO: 1084; SEQ ID NO: 1085; SEQ ID NO: 1086; SEQ ID NO: 1087; SEQ ID NO: 1088; SEQ ID NO: 1089; SEQ ID NO: 1090; SEQ ID NO: 1091; SEQ ID NO: 1092; SEQ ID NO: 1093; SEQ ID NO: 1094; SEQ ID NO: 1095; SEQ ID NO: 1096; SEQ ID NO: 1097; SEQ ID NO: 1098; SEQ ID NO: 1099; SEQ ID NO: 1100; SEQ ID NO: 1101; SEQ ID NO: 1102; SEQ ID NO: 1103; SEQ ID NO: 1104; SEQ ID NO: 1105; SEQ ID NO: 1106; SEQ ID NO: 1107; SEQ ID NO: 1108; SEQ ID NO: 1109; SEQ ID NO: 1110; SEQ ID NO: 1111; SEQ ID NO: 1112; SEQ ID NO: 1113; SEQ ID NO: 1114; SEQ ID NO: 1115; SEQ ID NO: 1116; SEQ ID NO: 1117; SEQ ID NO: 1118; SEQ ID NO: 1119; SEQ ID NO: 1120; SEQ ID NO: 1121; SEQ ID NO: 1122; SEQ ID NO: 1123; SEQ ID NO: 1124; SEQ ID NO: 1125; SEQ ID NO: 1126; SEQ ID NO: 1127; SEQ ID NO: 1128; SEQ ID NO: 1129; SEQ ID NO: 1130; SEQ ID NO: 1131; SEQ ID NO: 1132; SEQ ID NO: 1133; SEQ ID NO: 1134; SEQ ID NO: 1135; SEQ ID NO: 1136; SEQ ID NO: 1137; SEQ ID NO: 1138; SEQ ID NO: 1139; SEQ ID NO: 1140; SEQ ID NO: 1141; SEQ ID NO: 1142; SEQ ID NO: 1143; SEQ ID NO: 1144; SEQ ID NO: 1145; SEQ ID NO: 1146; SEQ ID NO: 1147; SEQ ID NO: 1148; SEQ ID NO: 1149; SEQ ID NO: 1150; SEQ ID NO: 1151; SEQ ID NO: 1152; SEQ ID NO: 1153; SEQ ID NO: 1154; SEQ ID NO: 1155; SEQ ID NO: 1156; SEQ ID NO: 1157; SEQ ID NO: 1158; SEQ ID NO: 1159; SEQ ID NO: 1160; SEQ ID NO: 1161; SEQ ID NO: 1162; SEQ ID NO: 1163; SEQ ID NO: 1164; SEQ ID NO: 1165; SEQ ID NO: 1166; SEQ ID NO: 1167; SEQ ID NO: 1168; SEQ ID NO: 1169; SEQ ID NO:1170; SEQ ID NO:1171; SEQ ID NO:1172; SEQ ID NO: 1173; SEQ ID NO: 1174; SEQ ID NO: 1175; SEQ ID NO: 1176; SEQ ID NO: 1177; SEQ ID NO: 1178; SEQ ID NO: 1179; SEQ ID NO: 1180; SEQ ID NO:1181; SEQ ID NO: 1182; SEQ ID NO: 1183; SEQ ID NO: 1184; SEQ ID NO: 1185; SEQ ID NO: 1'186; SEQ ID NO: 1187; SEQ ID NO: 1188; SEQ ID NO: 1189; SEQ ID NO: 1190; SEQ ID NO: 1191; SEQ ID NO: 1192; SEQ ID NO: 1193; SEQ ID NO: 1194; SEQ ID NO: 1195; SEQ ID NO: 1196; SEQ ID NO: 1197; SEQ ID NO: 1198; SEQ ID NO: 1199; SEQ ID NO: 1200; SEQ ID NO: 1201; SEQ ID NO: 1202; SEQ ID NO: 1203; SEQ ID NO: 1204; SEQ ID NO: 1205; SEQ ID NO: 1206; SEQ ID NO: 1207; SEQ ID NO: 1208; SEQ ID NO: 1209; SEQ ID NO: 1210; SEQ ID NO: 1121; SEQ ID NO: 1212; SEQ ID NO:1213; SEQ ID NO:1214; SEQ ID NO: 1215; SEQ ID NO:1216; SEQ ID NO: 1217; SEQ ID NO: 1218; SEQ ID NO: 1219; SEQ ID NO: 1220; SEQ ID NO: 1221; SEQ ID NO: 1222; SEQ ID NO: 1223; SEQ ID NO: 1224; SEQ ID NO: 1225; SEQ ID NO: 1226; SEQ ID NO: 1227; SEQ ID NO: 1228; SEQ ID NO: 1229; SEQ ID NO: 1230; SEQ ID NO: 1231; SEQ ID NO: 1232; SEQ ID NO: 1233; SEQ ID NO: 1234; SEQ ID NO: 1235; SEQ ID NO: 1236; SEQ ID NO: 1237; SEQ ID NO: 1238; SEQ ID NO: 1239; SEQ ID NO: 1240; SEQ ID NO: 1241; SEQ ID NO: 1242; SEQ ID NO: 1243; SEQ ID NO: 1244; SEQ ID NO: 1245; SEQ ID NO: 1246; SEQ ID NO: 1247; SEQ ID NO: 1248; SEQ ID NO: 1249; SEQ ID NO: 1250; SEQ ID NO: 1251; SEQ ID NO: 1252; SEQ ID NO: 1253; SEQ ID NO: 1254; SEQ ID NO: 1255; SEQ ID NO: 1256; SEQ ID NO: 1257; SEQ ID NO: 1258; SEQ ID NO: 1259; SEQ ID NO: 1260; SEQ ID NO: 1261; SEQ ID NO: 1262; SEQ ID NO: 1263; SEQ ID NO: 1264; SEQ ID NO: 1265; SEQ ID NO: 1266; SEQ ID NO: 1267; SEQ ID NO: 1268; SEQ ID NO: 1269; SEQ ID NO: 1270; SEQ ID NO: 1271; SEQ ID NO: 1272; SEQ ID NO: 1273; SEQ ID NO: 1274; SEQ ID NO: 1275; SEQ ID NO: 1276; SEQ ID NO: 1277; SEQ ID NO: 1278; SEQ ID NO: 1279; SEQ ID NO: 1280; SEQ ID NO: 1281; SEQ ID NO: 1282; SEQ ID NO: 1283; SEQ ID NO: 1284; SEQ ID NO: 1285; SEQ ID NO: 1286; SEQ ID NO: 1287; SEQ ID NO: 1288; SEQ ID NO: 1289; SEQ ID NO: 1290; SEQ ID NO: 1291; SEQ ID NO: 1292; SEQ ID NO: 1293; SEQ ID NO: 1294; SEQ ID NO: 1295; SEQ ID NO: 1296; SEQ ID NO: 1297; SEQ ID NO: 1298; SEQ ID NO: 1299; SEQ ID NO: 1300; SEQ ID NO: 1301; SEQ ID NO: 1302; SEQ ID NO: 1303; SEQ ID NO: 1304; SEQ ID NO: 1305; SEQ ID NO: 1306; SEQ ID NO: 1307; SEQ ID NO: 1308; SEQ ID NO: 1309; SEQ ID NO: 1310; SEQ ID NO: 1311; SEQ ID NO:1312; SEQ ID NO: 1313; SEQ ID NO:1314; SEQ ID NO:1315; SEQ ID NO: 1316; SEQ ID NO: 1317; SEQ ID NO: 1318; SEQ ID NO: 1319; SEQ ID NO: 1320; SEQ ID NO: 1321; SEQ ID NO: 1322; SEQ ID NO: 1323; SEQ ID NO: 1324; SEQ ID NO: 1325; SEQ ID NO: 1326; SEQ ID NO: 1327; SEQ ID NO: 1328; SEQ ID NO: 1329; SEQ ID NO: 1330; SEQ ID NO: 1331; SEQ ID NO: 1332; SEQ ID NO: 1333; SEQ ID NO: 1334; SEQ ID NO: 1335; SEQ ID NO: 1336; SEQ ID NO: 1337; SEQ ID NO: 1338; SEQ ID NO: 1339; SEQ ID NO: 1340; SEQ ID NO: 1341; SEQ ID NO: 1342; SEQ ID NO: 1343; SEQ ID NO: 1344; SEQ ID NO: 1345; SEQ ID NO: 1346; SEQ ID NO: 1347; SEQ ID NO: 1348; SEQ ID NO: 1349; SEQ ID NO: 1350; SEQ ID NO: 1351; SEQ ID NO: 1352; SEQ ID NO: 1353; SEQ ID NO: 1354; SEQ ID NO: 1355; SEQ ID NO: 1356; SEQ ID NO: 1357; SEQ ID NO: 1358; SEQ ID NO: 1359; SEQ ID NO: 1360; SEQ ID NO: 1361; SEQ ID NO: 1362; SEQ ID NO: 1363; SEQ ID NO: 1364; SEQ ID NO: 1365; SEQ ID NO: 1366; SEQ ID NO: 1367; SEQ ID NO: 1368; SEQ ID NO: 1369; SEQ ID NO: 1370; SEQ ID NO: 1371; SEQ ID NO: 1372; SEQ ID NO: 1373; SEQ ID NO: 1374; SEQ ID NO: 1375; SEQ ID NO: 1376; SEQ ID NO: 1377; SEQ ID NO: 1378; SEQ ID NO: 1379; SEQ ID NO: 1380; SEQ ID NO: 1381; SEQ ID NO: 1382; SEQ ID NO: 1383; SEQ ID NO: 1384; SEQ ID NO: 1385; SEQ ID NO: 1386; SEQ ID NO: 1387; SEQ ID NO: 1388; SEQ ID NO: 1389; SEQ ID NO: 1390; SEQ ID NO: 1391; SEQ ID NO: 1392; SEQ ID NO: 1393; SEQ ID NO: 1394; SEQ ID NO: 1395; SEQ ID NO: 1396; SEQ ID NO: 1397; SEQ ID NO: 1398; SEQ ID NO: 1399; SEQ ID NO: 1400; SEQ ID NO: 1401; SEQ ID NO: 1402; SEQ ID NO: 1403; SEQ ID NO: 1404; SEQ ID NO: 1405; SEQ ID NO: 1406; SEQ ID NO: 1407; SEQ ID NO: 1408; SEQ ID NO: 1409; SEQ ID NO: 1410; SEQ ID NO: 1411; SEQ ID NO: 1412; SEQ ID NO: 1413; SEQ ID NO: 1414; SEQ ID NO: 1415; SEQ ID NO: 1416; SEQ ID NO: 1417; SEQ ID NO: 1418; SEQ ID NO: 1419; SEQ ID NO: 1420; SEQ ID NO: 1421; SEQ ID NO: 1422; SEQ ID NO: 1423; SEQ ID NO: 1424; SEQ ID NO: 1425; SEQ ID NO: 1426; SEQ ID NO: 1427; SEQ ID NO: 1428; SEQ ID NO: 1429; SEQ ID NO: 1430; SEQ ID NO: 1431; SEQ ID NO: 1432; SEQ ID NO: 1433; SEQ ID NO: 1434; SEQ ID NO: 1435; SEQ ID NO: 1436; SEQ ID NO: 1437; SEQ ID NO: 1438; SEQ ID NO: 1439; SEQ ID NO: 1440; SEQ ID NO: 1441; SEQ ID NO: 1442; SEQ ID NO: 1443; SEQ ID NO: 1444; SEQ ID NO: 1445; SEQ ID NO: 1446; SEQ ID NO: 1447; SEQ ID NO: 1448; SEQ ID NO: 1449; SEQ ID NO: 1450; SEQ ID NO: 1451; SEQ ID NO: 1452; SEQ ID NO: 1453; SEQ ID NO: 1454; SEQ ID NO: 1455; SEQ ID NO: 1456; SEQ ID NO: 1457; SEQ ID NO: 1458; SEQ ID NO: 1459; SEQ ID NO: 1460; SEQ ID NO: 1461; SEQ ID NO: 1462; SEQ ID NO: 1463; SEQ ID NO: 1464; SEQ ID NO: 1465; SEQ ID NO: 1466; SEQ ID NO: 1467; SEQ ID NO: 1468; SEQ ID NO: 1469; SEQ ID NO: 1470; SEQ ID NO: 1471; SEQ ID NO: 1472; SEQ ID NO: 1473; SEQ ID NO: 1474; SEQ ID NO: 1475; SEQ ID NO: 1476; SEQ ID NO: 1477; SEQ ID NO: 1478; SEQ ID NO: 1479; SEQ ID NO: 1480; SEQ ID NO: 1481; SEQ ID NO: 1482; SEQ ID NO: 1483; SEQ ID NO: 1484; SEQ ID NO: 1485; SEQ ID NO: 1486; SEQ ID NO: 1487; SEQ ID NO: 1488; SEQ ID NO: 1489; SEQ ID NO: 1490; SEQ ID NO: 1491; SEQ ID NO: 1492; SEQ ID NO: 1493; SEQ ID NO: 1494; SEQ ID NO: 1495; SEQ ID NO: 1496; SEQ ID NO: 1497; SEQ ID NO: 1498; SEQ ID NO: 1499; SEQ ID NO: 1500; SEQ ID NO: 1501; SEQ ID NO: 1502; SEQ ID NO: 1503; SEQ ID NO: 1504; SEQ ID NO: 1505; SEQ ID NO: 1506; SEQ ID NO: 1507; SEQ ID NO: 1508; SEQ ID NO: 1509; SEQ ID NO: 1510; SEQ ID NO: 1511; SEQ ID NO: 1512, S2Q ID NO: 1513; SEQ ID NO: 1514; SEQ ID NO: 1515; SEQ ID NO: 1516; SEQ ID NO: 1517; SEQ ID NO: 1518; SEQ ID NO: 1519; SEQ ID NO: 1520; SEQ ID NO: 1521; SEQ ID NO: 1522; SEQ ID NO: 1523; SEQ ID NO: 1524; SEQ ID NO: 1525; SEQ ID NO: 1526; SEQ ID NO: 1527; SEQ ID NO: 1528; SEQ ID NO: 1529; SEQ ID NO: 1530; SEQ ID NO: 1531; SEQ ID NO: 1532; SEQ ID NO: 1533; SEQ ID NO: 1534; SEQ ID NO: 1535; SEQ ID NO: 1536; SEQ ID NO: 1537; SEQ ID NO: 1538; SEQ ID NO: 1539; SEQ ID NO: 1540; SEQ ID NO: 1541; SEQ ID NO: 1542; SEQ ID NO: 1543; SEQ ID NO: 1544; SEQ ID NO: 1545; SEQ ID NO: 1546; SEQ ID NO: 1547; SEQ ID NO: 1548; SEQ ID NO: 1549; SEQ ID NO: 1550; SEQ ID NO: 1551; SEQ ID NO: 1552; SEQ ID NO: 1553; SEQ ID NO: 1554; SEQ ID NO: 1555; SEQ ID NO: 1556; SEQ ID NO: 1557; SEQ ID NO: 1558; SEQ ID NO: 1559; SEQ ID NO: 1560; SEQ ID NO: 1561; SEQ ID NO: 1562; SEQ ID NO: 1563; SEQ ID NO: 1564; SEQ ID NO: 1565; SEQ ID NO: 1566; SEQ ID NO: 1567; SEQ ID NO: 1568; SEQ ID NO: 1569; SEQ ID NO: 1570; SEQ ID NO: 1571; SEQ ID NO: 1572; SEQ ID NO: 1573; SEQ ID NO: 1574; SEQ ID NO: 1575; and SEQ ID NO: 9525; SEQ ID NO: 9526; SEQ ID NO: 9527; SEQ ID NO: 9528; SEQ ID NO: 9529; SEQ ID NO: 9530; SEQ ID NO: 9531; SEQ ID NO: 9532; SEQ ID NO: 9533; SEQ ID NO: 9534; SEQ ID NO: 9535; SEQ ID NO: 9536; SEQ ID NO: 9537; SEQ ID NO: 9538; SEQ ID NO: 9539; SEQ ID NO: 9540; SEQ ID NO: 9541; SEQ ID NO: 9542; SEQ ID NO: 9543; SEQ ID NO: 9544; SEQ ID NO: 9545; SEQ ID NO: 9546; SEQ ID NO: 9547; SEQ ID NO: 9548; SEQ ID NO: 9549; SEQ ID NO: 9550; SEQ ID NO: 9551; SEQ ID NO: 9552; SEQ ID NO: 9553; SEQ ID NO: 9554; SEQ ID NO: 9555; SEQ ID NO: 9556; SEQ ID NO: 9557; SEQ ID NO: 9558; SEQ ID NO: 9559; SEQ ID NO: 9560; SEQ ID NO: 9561; SEQ ID NO: 9562; SEQ ID NO: 9563; SEQ ID NO: 9564; SEQ ID NO: 9565; SEQ ID NO: 9566; SEQ ID NO: 9567; SEQ ID NO: 9568; SEQ ID NO: 9569; SEQ ID NO: 9570; SEQ ID NO: 9571; SEQ ID NO: 9572; SEQ ID NO: 9573; SEQ ID NO: 9574; SEQ ID NO: 9575; SEQ ID NO: 9576; SEQ ID NO: 9577; SEQ ID NO: 9578; SEQ ID NO: 9579; SEQ ID NO: 9580; SEQ ID NO: 9581; SEQ ID NO: 9582; SEQ ID NO: 9583; SEQ ID NO: 9584; SEQ ID NO: 9585; SEQ ID NO: 9586; SEQ ID NO: 9587; SEQ ID NO: 9588; SEQ ID NO: 9589; SEQ ID NO: 9590; SEQ ID NO: 9591; SEQ ID NO:9592, or a complement thereof.

In one embodiment, the H. pylori cell envelope polypeptide or a fragment thereof is an H. pylori flagella-associated polypeptide or a fragment thereof encoded by a nucleic acid having a nucleotide sequence selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO:11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99, SEQ ID NO: 100; SEQ ID NO: 101; SEQ ID NO: 102; SEQ ID NO: 103; and SEQ ID NO: 9525; SEQ ID NO: 9526; SEQ ID NO: 9527, or a complement thereof. In another embodiment, the H. pylori cell envelope polypeptide or a fragment thereof is an H. pylori outer membrane polypeptide or a fragment thereof encoded by a nucleic acid having a nucleotide sequence selected from the group consisting of SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO: 117; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; SEQ ID NO: 186; SEQ ID NO: 187, SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; SEQ ID NO: 211; SEQ ID NO: 212; SEQ ID NO: 213; SEQ ID NO: 214; SEQ ID NO: 215; SEQ ID NO: 216; SEQ ID NO: 217; SEQ ID NO: 218; SEQ ID NO: 219; SEQ ID NO: 220; SEQ ID NO: 221; SEQ ID NO: 222; SEQ ID NO: 223; SEQ ID NO: 224; SEQ ID NO: 225; SEQ ID NO: 226; SEQ ID NO: 227; SEQ ID NO: 228; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 236; SEQ ID NO: 237; SEQ ID NO: 238; SEQ ID NO: 239; SEQ ID NO: 240; SEQ ID NO: 241; SEQ ID NO: 242; SEQ ID NO: 243; SEQ ID NO: 244; SEQ ID NO: 245; SEQ ID NO: 246; SEQ ID NO: 247; SEQ ID NO: 248; SEQ ID NO: 249; SEQ ID NO: 250; SEQ ID NO: 251; SEQ ID NO: 252; SEQ ID NO: 253; SEQ ID NO: 254; SEQ ID NO: 255; SEQ ID NO: 256; SEQ ID NO: 257; SEQ ID NO: 258; SEQ ID NO: 259; SEQ ID NO: 260; SEQ ID NO: 261; SEQ ID NO: 262; SEQ ID NO: 263; SEQ ID NO: 264; SEQ ID NO: 265; SEQ ID NO: 266; SEQ ID NO: 267; SEQ ID NO: 268; SEQ ID NO: 269; SEQ ID NO: 270; SEQ ID NO: 271; SEQ ID NO: 272; SEQ ID NO: 273; SEQ ID NO: 274; SEQ ID NO: 275; SEQ ID NO: 276; SEQ ID NO: 277; SEQ ID NO: 278; SEQ ID NO: 279; SEQ ID NO: 280; SEQ ID NO: 281; SEQ ID NO: 282; SEQ ID NO: 283; SEQ ID NO: 284; SEQ ID NO: 285; SEQ ID NO: 286; SEQ ID NO: 287; SEQ ID NO: 288; SEQ ID NO: 289; SEQ ID NO: 290; SEQ ID NO: 291; SEQ ID NO: 292; SEQ ID NO: 293; SEQ ID NO: 294; SEQ ID NO: 295; SEQ ID NO: 296; SEQ ID NO: 297; SEQ ID NO: 298; SEQ ID NO: 299; SEQ ID NO: 300; SEQ ID NO: 301; SEQ ID NO: 302; SEQ ID NO: 303; SEQ ID NO: 304; SEQ ID NO: 305; SEQ ID NO: 306; SEQ ID NO: 307; SEQ ID NO: 308; SEQ ID NO: 309; SEQ ID NO: 310; SEQ ID NO: 311; SEQ ID NO: 312; SEQ ID NO: 313; SEQ ID NO: 314; SEQ ID NO: 315; SEQ ID NO: 316; SEQ ID NO: 317; SEQ ID NO: 318; SEQ ID NO: 319; SEQ ID NO: 320; SEQ ID NO: 321; SEQ ID NO: 322; SEQ ID NO: 323; SEQ ID NO: 324; SEQ ID NO: 325; SEQ ID NO: 326; SEQ ID NO: 327; SEQ ID NO: 328; SEQ ID NO: 329; SEQ ID NO: 330; SEQ ID NO: 331; SEQ ID NO: 332; SEQ ID NO: 333; SEQ ID NO: 334; SEQ ID NO: 335; SEQ ID NO: 336; SEQ ID NO: 337; SEQ ID NO: 338; SEQ ID NO: 339; SEQ ID NO: 340; SEQ ID NO: 341; SEQ ID NO: 342; SEQ ID NO: 343; SEQ ID NO: 344; SEQ ID NO: 345; SEQ ID NO: 346; SEQ ID NO: 347; SEQ ID NO: 348; SEQ ID NO: 349; SEQ ID NO: 350; SEQ ID NO: 351; SEQ ID NO: 352; SEQ ID NO: 353; SEQ ID NO: 354; SEQ ID NO: 355; SEQ ID NO: 356; SEQ ID NO: 357; SEQ ID NO: 358; SEQ ID NO: 359; SEQ ID NO: 360; SEQ ID NO: 361; SEQ ID NO: 362; SEQ ID ND. 363; SEQ ID NO: 364; SEQ ID NO: 365; SEQ ID NO: 366; SEQ ID NO: 367; SEQ ID NO: 368; SEQ ID NO: 369; SEQ ID NO: 370; SEQ ID NO: 371; SEQ ID NO: 372; SEQ ID NO: 373; SEQ ID NO: 374; SEQ ID NO: 375; SEQ ID NO: 376; SEQ ID NO: 377; SEQ ID NO: 378; SEQ ID NO: 379; SEQ ID NO: 380; SEQ ID NO: 381; SEQ ID NO: 382; SEQ ID NO: 383; SEQ ID NO: 384; SEQ ID NO: 385; SEQ ID NO: 386; SEQ ID NO: 387; SEQ ID NO: 388; SEQ ID NO: 389; SEQ ID NO: 390; SEQ ID NO: 391; SEQ ID NO: 392; SEQ ID NO: 393; SEQ ID NO: 394; SEQ ID NO: 395; SEQ ID NO: 396; SEQ ID NO: 397; SEQ ID NO: 398; SEQ ID NO: 399; SEQ ID NO: 400; SEQ ID NO: 401; SEQ ID NO: 402; SEQ ID NO: 403; SEQ ID NO: 404; SEQ ID NO: 405; SEQ ID NO: 406; SEQ ID NO: 407; SEQ ID NO: 408; SEQ ID NO: 409; SEQ ID NO: 410; SEQ ID NO: 411; SEQ ID NO: 412; SEQ ID NO: 413; SEQ ID NO: 414; SEQ ID NO: 415; SEQ ID NO: 416; SEQ ID NO: 417; SEQ ID NO: 418; SEQ ID NO: 419; SEQ ID NO: 420; SEQ ID NO: 421; SEQ ID NO: 422; SEQ ID NO: 423; SEQ ID NO: 424; SEQ ID NO: 425; SEQ ID NO: 426; SEQ ID NO: 427; SEQ ID NO: 428; SEQ ID NO: 429; SEQ ID NO: 430; SEQ ID NO: 431; SEQ ID NO: 432; SEQ ID NO: 433; SEQ ID NO: 434; SEQ ID NO: 435; SEQ ID NO: 436; SEQ ID NO: 437; SEQ ID NO: 438; SEQ ID NO: 439; SEQ ID NO: 440; SEQ ID NO: 441; SEQ ID NO: 442; SEQ ID NO: 443; SEQ ID NO: 444; SEQ ID NO: 445; SEQ ID NO: 446; SEQ ID NO: 447; SEQ ID NO: 448; SEQ ID NO: 449; SEQ ID NO: 450; SEQ ID NO: 451; SEQ ID NO: 452; SEQ ID NO: 453; SEQ ID NO: 454; SEQ ID NO: 455; SEQ ID NO: 456; SEQ ID NO: 457; SEQ ID NO: 458; SEQ ID NO: 459; SEQ ID NO: 460; SEQ ID NO: 461; SEQ ID NO: 462; SEQ ID NO: 463; SEQ ID NO: 464; SEQ ID NO: 465; SEQ ID NO: 466; SEQ ID NO: 467; SEQ ID NO: 468; SEQ ID NO: 469; SEQ ID NO: 470; SEQ ID NO: 471; SEQ ID NO: 472; SEQ ID NO: 473; SEQ ID NO: 474; SEQ ID NO: 475; SEQ ID NO: 476; SEQ ID NO: 477; SEQ ID NO: 478; SEQ ID NO: 479; SEQ ID NO: 480; SEQ ID NO: 481; SEQ ID NO: 482; SEQ ID NO: 483; SEQ ID NO: 484; SEQ ID NO: 485; SEQ ID NO: 486; SEQ ID NO: 487; SEQ ID NO: 488; SEQ ID NO: 489; SEQ ID NO: 490; SEQ ID NO: 491; SEQ ID NO: 492; SEQ ID NO: 493; SEQ ID NO: 494; SEQ ID NO: 495; SEQ ID NO: 496; SEQ ID NO: 497; SEQ ID NO: 498; SEQ ID NO: 499; SEQ ID NO: 500; SEQ ID NO: 501; SEQ ID NO: 502; SEQ ID NO: 503; SEQ ID NO: 504; SEQ ID NO: 505; SEQ ID NO: 506; SEQ ID NO: 507; SEQ ID NO: 508; SEQ ID NO: 509; SEQ ID NO: 510; and SEQ ID NO: 9528; SEQ ID NO: 9529; SEQ ID NO: 9530; SEQ ID NO: 9531; SEQ ID NO: 9532; SEQ ID NO: 9533; SEQ ID NO: 9534; SEQ ID NO: 9535; SEQ ID NO: 9536, or a complement thereof.

In another embodiment, the H. pylori outer membrane polypeptide or a fragment thereof is an H. pylori polypeptide having a terminal phenylalanine residue or a fragment thereof encoded by a nucleic acid having a nucleotide sequence selected from the group consisting of SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO: 117; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; SEQ ID NO: 186; SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; SEQ ID NO: 211; SEQ ID NO: 212; SEQ ID NO: 213; SEQ ID NO: 214; SEQ ID NO: 215; SEQ ID NO: 216; SEQ ID NO: 217; SEQ ID NO: 218; SEQ ID NO: 219; SEQ ID NO: 220; SEQ ID NO: 221; SEQ ID NO: 222; SEQ ID NO: 223; SEQ ID NO: 224; SEQ ID NO: 225; SEQ ID NO: 226; SEQ ID NO: 227; SEQ ID NO: 228; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 236; SEQ ID NO: 237; SEQ ID NO: 238; SEQ ID NO: 239; SEQ ID NO: 240; SEQ ID NO: 241; SEQ ID NO: 242; SEQ ID NO: 243; SEQ ID NO: 244; SEQ ID NO: 245; SEQ ID NO: 246; SEQ ID NO: 247; SEQ ID NO: 248; SEQ ID NO: 249; SEQ ID NO: 250; SEQ ID NO: 251; SEQ ID NO: 252; SEQ ID NO: 253; SEQ ID NO: 254; SEQ ID NO: 255; SEQ ID NO: 256; SEQ ID NO: 257; SEQ ID NO: 258; SEQ ID NO: 259; SEQ ID NO: 260; SEQ ID NO: 261; SEQ ID NO: 262; SEQ ID NO: 263; SEQ ID NO: 264; SEQ ID NO: 265; SEQ ID NO: 266; SEQ ID NO: 267; SEQ ID NO: 268; SEQ ID NO: 269; SEQ ID NO: 270; SEQ ID NO: 271; SEQ ID NO: 272; SEQ ID NO: 273; SEQ ID NO: 274; SEQ ID NO: 275; SEQ ID NO: 276; SEQ ID NO: 277; SEQ ID NO: 278; SEQ ID NO: 279; SEQ ID NO: 280; SEQ ID NO: 281; SEQ ID NO: 282; SEQ ID NO: 283; SEQ ID NO: 284; SEQ ID NO: 285; SEQ ID NO: 286; SEQ ID NO: 287; SEQ ID NO: 288; SEQ ID NO: 289; SEQ ID NO: 290; SEQ ID NO: 291; SEQ ID NO: 292; SEQ ID NO: 293; SEQ ID NO: 294; SEQ ID NO: 295; SEQ ID NO: 296; SEQ ID NO: 297; SEQ ID NO: 298; SEQ ID NO: 299; SEQ ID NO: 300; SEQ ID NO: 301; SEQ ID NO: 302; SEQ ID NO: 303; SEQ ID NO: 304; SEQ ID NO: 305; SEQ ID NO: 306; SEQ ID NO: 307; SEQ ID NO: 308; SEQ ID NO: 309; SEQ ID NO: 310; SEQ ID NO: 311; SEQ ID NO: 312; SEQ ID NO: 313; SEQ ID NO:314; SEQ ID NO:315; SEQ ID NO:316; SEQ ID NO: 317; SEQ ID NO: 318; SEQ ID NO: 319; SEQ ID NO: 320; SEQ ID NO: 321; SEQ ID NO: 322; SEQ ID NO: 323; SEQ ID NO: 324; SEQ ID NO: 325; SEQ ID NO: 326; SEQ ID NO: 327; SEQ ID NO: 328; SEQ ID NO: 329; SEQ ID NO: 330; SEQ ID NO: 331; SEQ ID NO: 332; SEQ ID NO: 333; SEQ ID NO: 342; SEQ ID NO: 343; SEQ ID NO: 344; SEQ ID NO: 345; SEQ ID NO: 346; SEQ ID NO: 347; SEQ ID NO: 348; SEQ ID NO: 349; SEQ ID NO: 350; SEQ ID NO: 351; SEQ ID NO: 352; SEQ ID NO: 353; SEQ ID NO: 354; SEQ ID NO: 355; SEQ ID NO: 356; SEQ ID NO: 357; SEQ ID NO: 358; SEQ ID NO: 359; SEQ ID NO: 360; SEQ ID NO: 361; SEQ ID NO: 362; SEQ ID NO: 369; SEQ ID NO: 370; SEQ ID NO: 371; SEQ ID NO: 372; SEQ ID NO: 373; SEQ ID NO: 374; SEQ ID NO: 375; SEQ ID NO: 376; SEQ ID NO: 377; SEQ ID NO: 378; SEQ ID NO: 379; SEQ ID NO: 380; SEQ ID NO: 381; SEQ ID NO: 382; SEQ ID NO: 383; SEQ ID NO: 384; SEQ ID NO: 385; SEQ ID NO: 386; SEQ ID NO: 387; SEQ ID NO: 388; SEQ ID NO: 389; SEQ ID NO: 390; SEQ ID NO: 391; SEQ ID NO: 392; SEQ ID NO: 393; SEQ ID NO: 394; SEQ ID NO: 395; SEQ ID NO: 396; SEQ ID NO: 397; SEQ ID NO: 9528; SEQ ID NO: 9529; SEQ ID NO: 9530; SEQ ID NO: 9531; SEQ ID NO: 9532; SEQ ID NO: 9533; SEQ ID NO: 9534; SEQ ID NO: 9535, and SEQ ID NO: 9636 or a complement thereof.

In another embodiment, the H. pylori outer membrane polypeptide or a fragment thereof is an H. pylori polypeptide having a terminal phenylalanine residue and a C-terminal tyrosine cluster or a fragment thereof encoded by a nucleic acid having a nucleotide sequence selected from the group consisting of SEQ ID NO: 326; SEQ ID NO: 327; SEQ ID NO: 328; SEQ ID NO: 329; SEQ ID NO: 330; SEQ ID NO: 331; SEQ ID NO: 332; SEQ ID NO: 333; SEQ ID NO: 342; SEQ ID NO: 343; SEQ ID NO: 344; SEQ ID NO: 345; SEQ ID NO: 346; SEQ ID NO: 347; SEQ ID NO: 348; SEQ ID NO: 349; SEQ ID NO: 350; SEQ ID NO: 351; SEQ ID NO: 352; SEQ ID NO: 353; SEQ ID NO: 354; SEQ ID NO: 355; SEQ ID NO: 356; SEQ ID NO: 357; SEQ ID NO: 358; SEQ ID NO: 359; SEQ ID NO: 360; SEQ ID NO: 361; SEQ ID NO: 362; SEQ ID NO: 369; SEQ ID NO: 370; SEQ ID NO: 371; SEQ ID NO: 372; SEQ ID NO: 373; SEQ ID NO: 374; SEQ ID NO: 375; SEQ ID NO: 376; SEQ ID NO: 377; SEQ ID NO: 378; SEQ ID NO: 379; SEQ ID NO: 380; SEQ ID NO: 381; SEQ ID NO: 382; SEQ ID NO: 383; SEQ ID NO: 384; SEQ ID NO: 385; SEQ ID NO: 386; SEQ ID NO: 387; SEQ ID NO: 388; SEQ ID NO: 389; SEQ ID NO: 390; SEQ ID NO: 391; SEQ ID NO: 392; SEQ ID NO: 393; SEQ ID NO: 394; SEQ ID NO: 395; SEQ ID NO: 396; SEQ ID NO: 397 and SEQ ID NO: 9536, or a complement thereof.

In another embodiment, the H. pylori outer membrane polypeptide or a fragment thereof is an H. pylori polypeptide having a terminal tyrosine cluster motif or a fragment thereof encoded by a nucleic acid having a nucleotide sequence selected from the group consisting of SEQ ID NO: 326; SEQ ID NO: 327; SEQ ID NO: 328; SEQ ID NO: 329; SEQ ID NO: 330; SEQ ID NO: 331; SEQ ID NO: 332; SEQ ID NO: 333; SEQ ID NO: 334; SEQ ID NO: 335; SEQ ID NO: 336; SEQ ID NO: 337; SEQ ID NO: 338; SEQ ID NO: 339; SEQ ID NO: 340; SEQ ID NO: 341; SEQ ID NO: 342; SEQ ID NO: 343; SEQ ID NO: 344; SEQ ID NO: 345; SEQ ID NO: 346; SEQ ID NO: 347; SEQ ID NO: 348; SEQ ID NO: 349; SEQ ID NO: 350; SEQ ID NO: 351; SEQ ID NO: 352; SEQ ID NO: 353; SEQ ID NO: 354; SEQ ID NO: 355; SEQ ID NO: 356; SEQ ID NO: 357; SEQ ID NO: 358; SEQ ID NO: 359; SEQ ID NO: 360; SEQ ID NO: 361; SEQ ID NO: 362; SEQ ID NO: 363; SEQ ID NO: 364; SEQ ID NO: 365; SEQ ID NO: 366; SEQ ID NO: 367; SEQ ID NO: 368; SEQ ID NO: 369; SEQ ID NO: 370; SEQ ID NO: 371; SEQ ID NO: 372; SEQ ID NO: 373; SEQ ID NO: 374; SEQ ID NO: 375; SEQ ID NO: 376; SEQ ID NO: 377; SEQ ID NO: 378; SEQ ID NO: 379; SEQ ID NO: 380; SEQ ID NO: 381; SEQ ID NO: 382; SEQ ID NO: 383; SEQ ID NO: 384; SEQ ID NO: 385; SEQ ID NO: 386; SEQ ID NO: 387; SEQ ID NO: 388; SEQ ID NO: 389; SEQ ID NO: 390; SEQ ID NO: 391; SEQ ID NO: 392; SEQ ID NO: 393; SEQ ID NO: 394; SEQ ID NO: 395; SEQ ID NO: 396; SEQ ID NO: 397; SEQ ID NO: 398; SEQ ID NO: 399; SEQ ID NO: 400; SEQ ID NO: 401; SEQ ID NO: 402; SEQ ID NO: 403; SEQ ID NO: 404; SEQ ID NO: 405; SEQ ID NO: 406; SEQ ID NO: 407; SEQ ID NO: 408; SEQ ID NO: 409; SEQ ID NO:410; SEQ ID NO:411; SEQ ID NO:412; SEQ ID NO: 413; SEQ ID NO: 414; SEQ ID NO: 415; SEQ ID NO: 416; SEQ ID NO: 398; SEQ ID NO: 399; SEQ ID NO: 400; SEQ ID NO: 401; SEQ ID NO: 402; SEQ ID NO: 403; SEQ ID NO: 404; SEQ ID NO: 405; SEQ ID NO: 406; SEQ ID NO: 407; SEQ ID NO: 408; SEQ ID NO: 409; SEQ ID NO: 410; SEQ ID NO: 411; SEQ ID NO: 412; SEQ ID NO: 413; SEQ ID NO: 414; SEQ ID NO: 415; SEQ ID NO: 416; SEQ ID NO: 424; SEQ ID NO: 425 and SEQ ID NO: 9536, or a complement thereof.

In another embodiment, the H. pylori cell envelope polypeptide or a fragment thereof is an H. pylori inner membrane polypeptide or a fragment thereof encoded by a nucleic acid having a nucleotide sequence selected from the group consisting of SEQ ID NO: 511; SEQ ID NO: 512; SEQ ID NO: 513; SEQ ID NO: 514; SEQ ID NO: 515; SEQ ID NO: 516; SEQ ID NO: 517; SEQ ID NO: 518; SEQ ID NO: 519; SEQ ID NO: 520; SEQ ID NO: 521; SEQ ID NO: 522; SEQ ID NO: 523; SEQ ID NO: 524; SEQ ID NO: 525; SEQ ID NO: 526; SEQ ID NO: 527; SEQ ID NO: 528; SEQ ID NO: 529; SEQ ID NO: 530; SEQ ID NO: 531; SEQ ID NO: 532; SEQ ID NO: 533; SEQ ID NO: 534; SEQ ID NO: 535; SEQ ID NO: 536; SEQ ID NO: 537; SEQ ID NO: 538; SEQ ID NO: 539; SEQ ID NO: 540; SEQ ID NO: 541; SEQ ID NO: 542; SEQ ID NO: 543; SEQ ID NO: 544; SEQ ID NO: 545; SEQ ID NO: 546; SEQ ID NO: 547; SEQ ID NO: 548; SEQ ID NO: 549; SEQ ID NO: 550; SEQ ID NO: 551; SEQ ID NO: 552; SEQ ID NO: 553; SEQ ID NO: 554; SEQ ID NO: 555; SEQ ID NO: 556; SEQ ID NO: 557; SEQ ID NO: 558; SEQ ID NO: 559; SEQ ID NO: 560; SEQ ID NO: 561; SEQ ID NO: 562; SEQ ID NO: 563; SEQ ID NO: 564; SEQ ID NO: 565; SEQ ID NO: 566; SEQ ID NO: 567; SEQ ID NO: 568; SEQ ID NO: 569; SEQ ID NO: 570; SEQ ID NO: 571; SEQ ID NO: 572; SEQ ID NO: 573; SEQ ID NO: 574; SEQ ID NO: 575; SEQ ID NO: 576; SEQ ID NO: 577; SEQ ID NO: 578; SEQ ID NO: 579; SEQ ID NO: 580; SEQ ID NO: 581; SEQ ID NO: 582; SEQ ID NO: 583; SEQ ID NO: 584; SEQ ID NO: 585; SEQ ID NO: 586; SEQ ID NO: 587; SEQ ID NO: 588; SEQ ID NO: 589; SEQ ID NO: 590; SEQ ID NO: 591; SEQ ID NO: 592; SEQ ID NO: 593; SEQ ID NO: 594; SEQ ID NO: 595; SEQ ID NO: 596; SEQ ID NO: 597; SEQ ID NO: 598; SEQ ID NO: 599; SEQ ID NO: 600; SEQ ID NO: 601; SEQ ID NO: 602; SEQ ID NO: 603; SEQ ID NO: 604; SEQ ID NO: 605; SEQ ID NO: 606; SEQ ID NO: 607; SEQ ID NO: 608; SEQ ID NO: 609; SEQ ID NO: 610; SEQ ID NO: 611; SEQ ID NO: 612; SEQ ID NO: 613; SEQ ID NO: 614; SEQ ID NO: 615; SEQ ID NO: 616; SEQ ID NO: 617; SEQ ID NO: 618; SEQ ID NO: 619; SEQ ID NO: 620; SEQ ID NO: 621; SEQ ID NO: 622; SEQ ID NO: 623; SEQ ID NO: 624; SEQ ID NO: 625; SEQ ID NO: 626; SEQ ID NO: 627; SEQ ID NO: 628; SEQ ID NO: 629; SEQ ID NO: 630; SEQ ID NO: 631; SEQ ID NO: 632; SEQ ID NO: 633; SEQ ID NO: 634; SEQ ID NO: 635; SEQ ID NO: 636; SEQ ID NO: 637; SEQ ID NO: 638; SEQ ID NO: 639; SEQ ID NO: 640; SEQ ID NO: 641; SEQ ID NO: 642; SEQ ID NO: 643; SEQ ID NO: 644; SEQ ID NO: 645; SEQ ID NO: 646; SEQ ID NO: 647; SEQ ID NO: 648; SEQ ID NO: 649; SEQ ID NO: 650; SEQ ID NO: 651; SEQ ID NO: 652; SEQ ID NO: 653; SEQ ID NO: 654; SEQ ID NO: 655; SEQ ID NO: 656; SEQ ID NO: 657; SEQ ID NO: 658; SEQ ID NO: 659; SEQ ID NO: 660; SEQ ID NO: 661; SEQ ID NO: 662; SEQ ID NO: 663; SEQ ID NO: 664; SEQ ID NO: 665; SEQ ID NO: 666; SEQ ID NO: 667; SEQ ID NO: 668; SEQ ID NO: 669; SEQ ID NO: 670; SEQ ID NO: 671; SEQ ID NO: 672; SEQ ID NO: 673; SEQ ID NO: 674; SEQ ID NO: 675; SEQ ID NO: 676; SEQ ID NO: 677; SEQ ID NO: 678; SEQ ID NO: 679; SEQ ID NO: 680; SEQ ID NO: 681; SEQ ID NO: 682; SEQ ID NO: 683; SEQ ID NO: 684; SEQ ID NO: 685; SEQ ID NO: 686; SEQ ID NO: 687; SEQ ID NO: 688; SEQ ID NO: 689; SEQ ID NO: 690; SEQ ID NO: 691; SEQ ID NO: 692; SEQ ID NO: 693; SEQ ID NO: 694; SEQ ID NO: 695; SEQ ID NO: 696; SEQ ID NO: 697; SEQ ID NO: 698; SEQ ID NO: 699; SEQ ID NO: 700; SEQ ID NO: 701; SEQ ID NO: 702; SEQ ID NO: 703; SEQ ID NO: 704; SEQ ID NO: 705; SEQ ID NO: 706; SEQ ID NO: 707; SEQ ID NO: 708; SEQ ID NO: 709; SEQ ID NO: 710; SEQ ID NO: 711; SEQ ID NO: 712; SEQ ID NO: 713; SEQ ID NO: 714; SEQ ID NO: 715; SEQ ID NO: 716; SEQ ID NO: 717; SEQ ID NO: 718; SEQ ID NO: 719; SEQ ID NO: 720; SEQ ID NO: 721; SEQ ID NO: 722; SEQ ID NO: 723; SEQ ID NO: 724; SEQ ID NO: 725; SEQ ID NO: 726; SEQ ID NO: 727; SEQ ID NO: 728; SEQ ID NO: 729; SEQ ID NO: 730; SEQ ID NO: 731; SEQ ID NO: 732; SEQ ID NO: 733; SEQ ID NO: 734; SEQ ID NO: 735; SEQ ID NO: 736; SEQ ID NO: 737; SEQ ID NO: 738; SEQ ID NO: 739; SEQ ID NO: 740; SEQ ID NO: 741; SEQ ID NO: 742; SEQ ID NO: 743; SEQ ID NO: 744; SEQ ID NO: 745; SEQ ID NO: 746; SEQ ID NO: 747; SEQ ID NO: 748; SEQ ID NO: 749; SEQ ID NO: 750; SEQ ID NO: 751; SEQ ID NO: 752; SEQ ID NO: 753; SEQ ID NO: 754; SEQ ID NO: 755; SEQ ID NO: 756; SEQ ID NO: 757; SEQ ID NO: 758; SEQ ID NO: 759; SEQ ID NO: 760; SEQ ID NO: 761; SEQ ID NO: 762; SEQ ID NO: 763; SEQ ID NO: 764; SEQ ID NO: 765; SEQ ID NO: 766; SEQ ID NO: 767; SEQ ID NO: 768; SEQ ID NO: 769; SEQ ID NO: 770; SEQ ID NO: 771; SEQ ID NO: 772; SEQ ID NO: 773; SEQ ID NO: 774; SEQ ID NO: 775; SEQ ID NO: 776; SEQ ID NO: 777; SEQ ID NO: 778; SEQ ID NO: 779; SEQ ID NO: 780; SEQ ID NO: 781; SEQ ID NO: 782; SEQ ID NO: 783; SEQ ID NO: 784; SEQ ID NO: 785; SEQ ID NO: 786; SEQ ID NO: 787; SEQ ID NO: 788; SEQ ID NO: 789; SEQ ID NO: 790; SEQ ID NO: 791; SEQ ID NO: 792; SEQ ID NO: 793; SEQ ID NO: 794; SEQ ID NO: 795; SEQ ID NO: 796; SEQ ID NO: 797; SEQ ID NO: 798; SEQ ID NO: 799; SEQ ID NO: 800; SEQ ID NO: 801; SEQ ID NO: 802; SEQ ID NO: 803; SEQ ID NO: 804; SEQ ID NO: 805; SEQ ID NO: 806; SEQ ID NO: 807; SEQ ID NO: 808; SEQ ID NO: 809; SEQ ID NO: 810; SEQ ID NO: 811; SEQ ID NO: 812; SEQ ID NO: 813; SEQ ID NO: 814; SEQ ID NO: 815; SEQ ID NO: 816; SEQ ID NO: 817; SEQ ID NO: 818; SEQ ID NO: 819; SEQ ID NO: 820; SEQ ID NO: 821; SEQ ID NO: 822; SEQ ID NO: 823; SEQ ID NO: 824; SEQ ID NO: 825; SEQ ID NO: 826; SEQ ID NO: 827; SEQ ID NO: 828; SEQ ID NO: 829; SEQ ID NO: 830; SEQ ID NO: 831; SEQ ID NO: 832; SEQ ID NO: 833; SEQ ID NO: 834; SEQ ID NO: 835; SEQ ID NO: 836; SEQ ID NO: 837; SEQ ID NO: 838; SEQ ID NO: 839; SEQ ID NO: 840; SEQ ID NO: 841; SEQ ID NO: 842; SEQ ID NO: 843; SEQ ID NO: 844; SEQ ID NO: 845; SEQ ID NO: 846; SEQ ID NO: 847; SEQ ID NO: 848; SEQ ID NO: 849; SEQ ID NO: 850; SEQ ID NO: 851; SEQ ID NO: 852; SEQ ID NO: 853; SEQ ID NO: 854; SEQ ID NO: 855; SEQ ID NO: 856; SEQ ID NO: 857; SEQ ID NO: 858; SEQ ID NO: 859; SEQ ID NO: 860; SEQ ID NO: 861; SEQ ID NO: 862; SEQ ID NO: 863; SEQ ID NO: 864; SEQ ID NO: 865; SEQ ID NO: 866; SEQ ID NO: 867; SEQ ID NO: 868; SEQ ID NO: 869; SEQ ID NO: 870; SEQ ID NO: 871; SEQ ID NO: 872; SEQ ID NO: 873; SEQ ID NO: 874; SEQ ID NO: 875; SEQ ID NO: 876; SEQ ID NO: 877; SEQ ID NO: 878; SEQ ID NO: 879; SEQ ID NO: 880; SEQ ID NO: 881; SEQ ID NO: 882; SEQ ID NO: 883; SEQ ID NO: 884; SEQ ID NO: 885; SEQ ID NO: 886; SEQ ID NO: 887; SEQ ID NO: 888; SEQ ID NO: 889; SEQ ID NO: 890; SEQ ID NO: 891; SEQ ID NO: 892; SEQ ID NO:893; SEQ ID NO: 894; SEQ ID NO: 895; SEQ ID NO: 896; SEQ ID NO: 897; SEQ ID NO: 898; SEQ ID NO: 899; SEQ ID NO: 900; SEQ ID NO: 901; SEQ ID NO: 902; SEQ ID NO: 903; SEQ ID NO: 904; SEQ ID NO: 905; SEQ ID NO: 906; SEQ ID NO: 907; SEQ ID NO: 908; SEQ ID NO: 909; SEQ ID NO: 910; SEQ ID NO: 911; SEQ ID NO: 912; SEQ ID NO: 913; SEQ ID NO: 914; SEQ ID NO: 915; SEQ ID NO: 916; SEQ ID NO: 917; SEQ ID NO: 918; SEQ ID NO: 919; SEQ ID NO: 920; SEQ ID NO: 921; SEQ ID NO: 922; SEQ ID NO: 923; SEQ ID NO: 924; SEQ ID NO: 925; SEQ ID NO: 926; SEQ ID NO: 927; SEQ ID NO: 928; SEQ ID NO: 929; SEQ ID NO: 930; SEQ ID NO: 931; SEQ ID NO: 932; SEQ ID NO: 933; SEQ ID NO: 934; SEQ ID NO: 935; SEQ ID NO: 936; SEQ ID NO: 937; SEQ ID NO: 938; SEQ ID NO: 939; SEQ ID NO: 940; SEQ ID NO: 941; SEQ ID NO: 942; SEQ ID NO: 943; SEQ ID NO: 944; SEQ ID NO: 945; SEQ ID NO: 946; SEQ ID NO: 947; SEQ ID NO: 948; SEQ ID NO: 949; SEQ ID NO: 950; SEQ ID NO: 951; SEQ ID NO: 952; SEQ ID NO: 953; SEQ ID NO: 954; SEQ ID NO: 955; SEQ ID NO: 956; SEQ ID NO: 957; SEQ ID NO: 958; SEQ ID NO: 959; SEQ ID NO: 960; SEQ ID NO: 961; SEQ ID NO: 962; SEQ ID NO: 963; SEQ ID NO: 964; SEQ ID NO: 965; SEQ ID NO: 966; SEQ ID NO: 967; SEQ ID NO: 968; SEQ ID NO: 969; SEQ ID NO: 970; SEQ ID NO: 971; SEQ ID NO: 972; SEQ ID NO: 973; SEQ ID NO: 974; SEQ ID NO: 975; SEQ ID NO: 976; SEQ ID NO: 977; SEQ ID NO: 978; SEQ ID NO: 979; SEQ ID NO: 980; SEQ ID NO: 981; SEQ ID NO: 982; SEQ ID NO: 983; SEQ ID NO: 984; SEQ ID NO: 985; SEQ ID NO: 986; SEQ ID NO: 987; SEQ ID NO: 988; SEQ ID NO: 989; SEQ ID NO: 990; SEQ ID NO: 991; SEQ ID NO: 992; SEQ ID NO: 993; SEQ ID NO: 994; SEQ ID NO: 995; SEQ ID NO: 996; SEQ ID NO: 997; SEQ ID NO: 998; SEQ ID NO: 999; SEQ ID NO: 1000; SEQ ID NO: 1001; SEQ ID NO: 1002; SEQ ID NO: 1003; SEQ ID NO: 1004; SEQ ID NO: 1005; SEQ ID NO: 1006; SEQ ID NO: 1007; SEQ ID NO: 1008; SEQ ID NO: 1009; SEQ ID NO: 1010; SEQ ID NO: 1011; SEQ ID NO: 1012; SEQ ID NO: 1013; SEQ ID NO: 1014; SEQ ID NO: 1015; SEQ ID NO: 1016; SEQ ID NO: 1017; SEQ ID NO: 1018; SEQ ID NO: 1019; SEQ ID NO: 1020; SEQ ID NO: 1021; SEQ ID NO: 1022; SEQ ID NO: 1023; SEQ ID NO: 1024; SEQ ID NO: 1025; SEQ ID NO: 1026; SEQ ID NO: 1027; SEQ ID NO: 1028; SEQ ID NO: 1029; SEQ ID NO: 1030; SEQ ID NO: 1031; SEQ ID NO: 1032; SEQ ID NO: 1033; SEQ ID NO: 1034; SEQ ID NO: 1035; SEQ ID NO: 1036; SEQ ID NO: 1037; SEQ ID NO: 1038; SEQ ID NO: 1039; SEQ ID NO: 1040; SEQ ID NO: 1041; SEQ ID NO: 1042; SEQ ID NO: 1043; SEQ ID NO: 1044; SEQ ID NO: 1045; SEQ ID NO: 1046; SEQ ID NO: 1047; SEQ ID NO: 1048; SEQ ID NO: 1049; SEQ ID NO: 1050; SEQ ID NO: 1051; SEQ ID NO: 1052; SEQ ID NO: 1053; SEQ ID NO: 1054; SEQ ID NO: 1055; SEQ ID ND 1056; SEQ ID NO: 1057; SEQ ID NO: 1058; SEQ ID NO: 1059; SEQ ID NO: 1060; SEQ ID NO: 1061; SEQ ID NO: 1062; SEQ ID NO: 1063; SEQ ID NO: 1064; SEQ ID NO: 1065; SEQ ID NO: 1066; SEQ ID NO: 1067; SEQ ID NO: 1068; SEQ ID NO: 1069; SEQ ID NO: 1070; SEQ ID NO: 1071; SEQ ID NO: 1072; SEQ ID NO: 1073; SEQ ID NO: 1074; SEQ ID NO: 1075; SEQ ID NO: 1076; SEQ ID NO: 1077; SEQ ID NO: 1078; SEQ ID NO: 1079; SEQ ID NO: 1080; SEQ ID NO: 1081; SEQ ID NO: 1082; SEQ ID NO: 1083; SEQ ID NO: 1084; SEQ ID NO: 1085; SEQ ID NO: 1086; SEQ ID NO: 1087; SEQ ID NO: 1088; SEQ ID NO: 1089; SEQ ID NO: 1090; SEQ ID NO: 1091; SEQ ID NO: 1092; SEQ ID NO: 1093; SEQ ID NO: 1094; SEQ ID NO: 1095; SEQ ID NO: 1096; SEQ ID NO: 1097; SEQ ID NO: 1098; SEQ ID NO: 1099; SEQ ID NO: 1100; SEQ ID NO: 1101; SEQ ID NO: 1102; SEQ ID NO: 1103; SEQ ID NO: 1104; SEQ ID NO: 1105; SEQ ID NO: 1106; SEQ ID NO: 1107; SEQ ID NO: 1108; SEQ ID NO: 1109; SEQ ID NO: 1110; SEQ ID NO: 1111; SEQ ID NO: 1112; SEQ ID NO: 1113; SEQ ID NO: 1114; SEQ ID NO: 1115; SEQ ID NO: 1116; SEQ ID NO: 1117; SEQ ID NO: 1118; SEQ ID NO: 1119; SEQ ID NO: 1120; SEQ ID NO: 1121; SEQ ID NO: 1122; SEQ ID NO: 1123; SEQ ID NO: 1124; SEQ ID NO: 1125; SEQ ID NO: 1126; SEQ ID NO: 1127; SEQ ID NO: 1128; SEQ ID NO: 1129; SEQ ID NO: 1130; SEQ ID NO: 1131; SEQ ID NO: 1132; SEQ ID NO: 1133; SEQ ID NO: 1134; SEQ ID NO: 1135; SEQ ID NO: 1136; SEQ ID NO: 1137; SEQ ID NO: 1138; SEQ ID NO: 1139; SEQ ID NO: 1140; SEQ ID NO: 1141; SEQ ID NO: 1142; SEQ ID NO: 1143; SEQ ID NO: 1144; SEQ ID NO: 1145; SEQ ID NO: 1146; SEQ ID NO: 1147; SEQ ID NO: 1148; SEQ ID NO: 1149; SEQ ID NO: 1150; SEQ ID NO: 1151; SEQ ID NO: 1152; SEQ ID NO: 1153; SEQ ID NO: 1154; SEQ ID NO: 1155; SEQ ID NO: 1156; SEQ ID NO: 1157; SEQ ID NO: 1158; SEQ ID NO: 1159; SEQ ID NO: 1160; SEQ ID NO: 1161; SEQ ID NO: 1162; SEQ ID NO: 1163; SEQ ID NO: 1164; SEQ ID NO: 1165; SEQ ID NO: 1166; SEQ ID NO: 1167; SEQ ID NO: 1168; SEQ ID NO: 1169; SEQ ID NO: 1170; SEQ ID NO: 1171; SEQ ID NO: 1172; SEQ ID NO: 1173; SEQ ID NO: 1174; SEQ ID NO: 1175; SEQ ID NO: 1176; SEQ ID NO: 1177; SEQ ID NO: 1178; SEQ ID NO: 1179; SEQ ID NO: 1180; SEQ ID NO: 1181; SEQ ID NO: 1182; SEQ ID NO: 1183; SEQ ID NO: 1184; SEQ ID NO: 1185; SEQ ID NO: 1186; SEQ ID NO: 1187; SEQ ID NO: 1188; SEQ ID NO: 118Q9 SEQ ID NO: 1190; SEQ ID NO: 1191; SEQ ID NO: 1192; SEQ ID NO: 1193; SEQ ID NO: 1194; SEQ ID NO: 1195; SEQ ID NO: 1196; SEQ ID NO: 1197; SEQ ID NO: 1198; SEQ ID NO: 1199; SEQ ID NO: 1200; SEQ ID NO: 1201; SEQ ID NO: 1202; SEQ ID NO: 1203; SEQ ID NO: 1204; SEQ ID NO: 1205; SEQ ID NO: 1206; SEQ ID NO: 1207; SEQ ID NO: 1208; SEQ ID NO: 1209; SEQ ID NO: 1210; SEQ ID NO: ¹²¹I; SEQ ID NO: 1212; SEQ ID NO: 1213; SEQ ID NO: 1214; SEQ ID NO: 1215; SEQ ID NO: 1216; SEQ ID NO: 1217; SEQ ID NO: 1218; SEQ ID NO: 1219; SEQ ID NO: 1220; SEQ ID NO: 1221; SEQ ID NO: 1222; SEQ ID NO: 1223; SEQ ID NO: 1224; SEQ ID NO: 1225; SEQ ID NO: 1226; SEQ ID NO: 1227; SEQ ID NO: 1228; SEQ ID NO: 1229; SEQ ID NO: 1230; SEQ ID NO: 1231; SEQ ID NO: 1232; SEQ ID NO: 1233; SEQ ID NO: 1234; SEQ ID NO: 1235; SEQ ID NO: 1236; SEQ ID NO: 1237; SEQ ID NO: 1238; SEQ ID NO: 1239; SEQ ID NO: 1240; SEQ ID NO: 1241; SEQ ID NO: 1242; SEQ ID NO: 1243; SEQ ID NO: 1244; SEQ ID NO: 1245; SEQ ID NO: 1246; SEQ ID NO: 1247; SEQ ID NO: 1248; SEQ ID NO: 1249; SEQ ID NO: 1250; SEQ ID NO: 1251; SEQ ID NO: 1252; SEQ ID NO: 1253; SEQ ID NO: 1254; SEQ ID NO: 1255; SEQ ID NO: 1256; SEQ ID NO: 1257; SEQ ID NO: 1258; SEQ ID NO: 1259; SEQ ID NO: 1260; SEQ ID NO: 1261; SEQ ID NO: 1262; SEQ ID NO: 1263; SEQ ID NO: 1264; SEQ ID NO: 1265; SEQ ID NO: 1266; SEQ ID NO: 1267; SEQ ID NO: 1268; SEQ ID NO: 1269; SEQ ID NO: 1270; SEQ ID NO: 1271; SEQ ID NO: 1272; SEQ ID NO: 1273; SEQ ID NO: 1274; SEQ ID NO: 1275; SEQ ID NO: 1276; SEQ ID NO: 1277; SEQ ID NO: 1278; SEQ ID NO: 1279; SEQ ID NO: 1280; SEQ ID NO: 1281; SEQ ID NO: 1282; SEQ ID NO: 1283; SEQ ID NO: 1284; SEQ ID NO: 1285; SEQ ID NO: 1286; SEQ ID NO: 1287; SEQ ID NO: 1288; SEQ ID NO: 1289; SEQ ID NO: 1290; SEQ ID NO: 1291; SEQ ID NO: 1292; SEQ ID NO: 1293; SEQ ID NO: 1294; SEQ ID NO: 1295; SEQ ID NO: 1296; SEQ ID NO: 1297; SEQ ID NO: 1298; SEQ ID NO: 1299; SEQ ID NO: 1300; SEQ ID NO: 1301; SEQ ID NO: 1302; SEQ ID NO: 1303; SEQ ID NO: 1304; SEQ ID NO: 1305; SEQ ID NO: 1306; SEQ ID NO: 1307; SEQ ID NO: 1308; SEQ ID NO: 1309; SEQ ID NO: 1310; SEQ ID NO: ¹³¹I; SEQ ID NO: 1312; SEQ ID NO: 1313; SEQ ID NO: 1314; SEQ ID NO: 1315; SEQ ID NO: 1316; SEQ ID NO: 1317; SEQ ID NO: 1318; SEQ ID NO: 1319; SEQ ID NO: 1320; SEQ ID NO: 1321; SEQ ID NO: 1322; SEQ ID NO: 1323; SEQ ID NO: 1324; SEQ ID NO: 1325; SEQ ID NO: 1326; SEQ ID NO: 1327; SEQ ID NO: 1328; SEQ ID NO: 1329; SEQ ID NO: 1330; SEQ ID NO: 1331; SEQ ID NO: 1332; SEQ ID NO: 1333; SEQ ID NO: 1334; SEQ ID NO: 1335; SEQ ID NO: 1336; SEQ ID NO: 1337; SEQ ID NO: 1338; SEQ ID NO: 1339; SEQ ID NO: 1340; SEQ ID NO: 1341; SEQ ID NO: 1342; SEQ ID NO: 1343; SEQ ID NO: 1344; SEQ ID NO: 1345; SEQ ID NO: 1346; SEQ ID NO: 1347; SEQ ID NO: 1348; SEQ ID NO: 1349; SEQ ID NO: 1350; SEQ ID NO: ¹³⁵I; SEQ ID NO: 1352; SEQ ID NO: 1353; SEQ ID NO: 1354; SEQ ID NO: 1355; SEQ ID NO: 1356; SEQ ID NO: 1357; SEQ ID NO: 1358; SEQ ID NO: 1359; SEQ ID NO: 1360; SEQ ID NO: 1361; SEQ ID NO: 1362; SEQ ID NO: 1363; SEQ ID NO: 1364; SEQ ID NO: 1365; SEQ ID NO: 1366; SEQ ID NO: 1367; SEQ ID NO: 1368; SEQ ID NO: 1369; SEQ ID NO: 1370; SEQ ID NO: 1371; SEQ ID NO: 1372; SEQ ID NO: 1373; SEQ ID NO: 1374; SEQ ID NO: 1375; SEQ ID NO: 1376; SEQ ID NO: 1377; SEQ ID NO: 1378; SEQ ID NO: 1379; SEQ ID NO: 1380; SEQ ID NO: 1381; SEQ ID NO: 1382; SEQ ID NO: 1383; SEQ ID NO: 1384; SEQ ID NO: 1385; SEQ ID NO: 1386; SEQ ID NO: 1387; SEQ ID NO: 1388; SEQ ID NO: 1389; SEQ ID NO: 1390; SEQ ID NO: 1391; SEQ ID NO: 1392; SEQ ID NO: 1393; SEQ ID NO: 1394; SEQ ID NO: 1395; SEQ ID NO: 1396; SEQ ID NO: 1397; SEQ ID NO: 1398; SEQ ID NO: 1399; SEQ ID NO: 1400; SEQ ID NO: 1401; SEQ ID NO: 1402; SEQ ID NO: 1403; SEQ ID NO: 1404; SEQ ID NO: 1405; SEQ ID NO: 1406; SEQ ID NO: 1407; SEQ ID NO: 1408; SEQ ID NO: 1409; SEQ ID NO: 1410; SEQ ID NO: 1411; SEQ ID NO: 1412; SEQ ID NO: 1413; SEQ ID NO: 1414; SEQ ID NO: 1415; SEQ ID NO: 1416; SEQ ID NO: 1417; SEQ ID NO: 1418; SEQ ID NO: 1419; SEQ ID NO: 1420; SEQ ID NO: 1421; SEQ ID NO: 1422; SEQ ID NO: 1423; SEQ ID NO: 1424; SEQ ID NO: 1425; SEQ ID NO: 1426; SEQ ID NO: 1427; SEQ ID NO: 1428; SEQ ID NO: 1429; SEQ ID NO: 1430; SEQ ID NO: 1431; SEQ ID NO: 1432; SEQ ID NO: 1433; SEQ ID NO: 1434; SEQ ID NO: 1435; SEQ ID NO: 1436; SEQ ID NO: 1437; SEQ ID NO: 1438; SEQ ID NO: 1439; SEQ ID NO: 1440; SEQ ID NO: 1441; SEQ ID NO: 1442; SEQ ID NO: 1443; SEQ ID NO: 1444; SEQ ID NO: 1445; SEQ ID NO: 1446; SEQ ID NO: 1447; SEQ ID NO: 1448; SEQ ID NO: 1449; SEQ ID NO: 1450; SEQ ID NO: 1451; SEQ ID NO: 1452; SEQ ID NO: 1453; SEQ ID NO: 1454; SEQ ID NO: 1455; SEQ ID NO: 1456; SEQ ID NO: 1457; SEQ ID NO: 1458; SEQ ID NO: 1459; SEQ ID NO: 1460; SEQ ID NO: 1461; SEQ ID NO: 1462; SEQ ID NO: 1463; SEQ ID NO: 1464; SEQ ID NO: 1465; SEQ ID NO: 1466; SEQ ID NO: 1467; SEQ ID NO: 1468; SEQ ID NO: 1469; SEQ ID NO: 1470; SEQ ID NO: 1471; SEQ ID NO: 1472; SEQ ID NO: 1473; SEQ ID NO: 1474; SEQ ID NO: 1475; SEQ ID NO: 1476; SEQ ID NO: 1477; SEQ ID NO: 1478; SEQ ID NO: 1479; SEQ ID NO: 1480; SEQ ID NO: 1481; SEQ ID NO: 1482; SEQ ID NO: 1483; SEQ ID NO: 1484; SEQ ID NO: 1485; SEQ ID NO: 1486; SEQ ID NO: 1487; SEQ ID NO: 1488; SEQ ID NO: 1489; SEQ ID NO: 1490; SEQ ID NO: 1491; SEQ ID NO: 1492; SEQ ID NO: 1493; SEQ ID NO: 1494; SEQ ID NO: 1495; SEQ ID NO: 1496; SEQ ID NO: 1497; SEQ ID NO: 1498; SEQ ID NO: 1499; SEQ ID NO: 1500; SEQ ID NO: 1501; SEQ ID NO: 1502; SEQ ID NO: 1503; SEQ ID NO: 1504; SEQ ID NO: 1505; SEQ ID NO: 1506; SEQ ID NO: 1507; SEQ ID NO: 1508; SEQ ID NO: 1509; SEQ ID NO: 1510; SEQ ID NO: 1511; SEQ ID NO: 1512; SEQ ID NO: 1513; SEQ ID NO: 1514; and SEQ ID NO: 9537; SEQ ID NO: 9538; SEQ ID NO: 9539; SEQ ID NO: 9540; SEQ ID NO: 9541; SEQ ID NO: 9542; SEQ ID NO: 9543; SEQ ID NO: 9544; SEQ ID NO: 9545; SEQ ID NO: 9546; SEQ ID NO: 9547; SEQ ID NO: 9548; SEQ ID NO: 9549; SEQ ID NO: 9550; SEQ ID NO: 9551; SEQ ID NO: 9552; SEQ ID NO: 9553; SEQ ID NO: 9554; SEQ ID NO: 9555; SEQ ID NO: 9556; SEQ ID NO: 9557; SEQ ID NO: 9558; SEQ ID NO: 9559; SEQ ID NO: 9560; SEQ ID NO: 9561; SEQ ID NO: 9562; SEQ ID NO: 9563; SEQ ID NO: 9564; SEQ ID NO: 9565; SEQ ID NO: 9566; SEQ ID NO: 9567; SEQ ID NO: 9568; SEQ ID NO: 9569; SEQ ID NO: 9570; SEQ ID NO: 9571; SEQ ID NO: 9572; SEQ ID NO: 9573; SEQ ID NO: 9574; SEQ ID NO: 9575; SEQ ID NO: 9576; SEQ ID NO: 9577; SEQ ID NO: 9578; SEQ ID NO: 9579; SEQ ID NO: 9580; SEQ ID NO: 9581; SEQ ID NO: 9582; SEQ ID NO: 9583; SEQ ID NO: 9584; SEQ ID NO: 9585; SEQ ID NO: 9586; SEQ ID NO: 9587; SEQ ID NO: 9588; SEQ ID NO: 9589; SEQ ID NO: 9590; and SEQ ID NO: 9591, or a complement thereof.

In another embodiment, the H. pylori cell envelope polypeptide or a fragment thereof is an H. pylori inner membrane polypeptide or a fragment thereof involved in transport encoded by a nucleic acid having a nucleotide sequence selected from the group consisting of SEQ ID NO: 511; SEQ ID NO: 512; SEQ ID NO: 513; SEQ ID NO: 514; SEQ ID NO: 515; SEQ ID NO: 516; SEQ ID NO: 517; SEQ ID NO: 518; SEQ ID NO: 519; SEQ ID NO: 520; SEQ ID NO: 521; SEQ ID NO: 522; SEQ ID NO: 523; SEQ ID NO: 524; SEQ ID NO: 525; SEQ ID NO: 526; SEQ ID NO: 527; SEQ ID NO: 528; SEQ ID NO: 529; SEQ ID NO: 530; SEQ ID NO: 531; SEQ ID NO: 532; SEQ ID NO: 533; SEQ ID NO: 534; SEQ ID NO: 535; SEQ ID NO: 536; SEQ ID NO: 537; SEQ ID NO: 538; SEQ ID NO: 539; SEQ ID NO: 540; SEQ ID NO: 541; SEQ ID NO: 542; SEQ ID NO: 543; SEQ ID NO: 544; SEQ ID NO: 545; SEQ ID NO: 546; SEQ ID NO: 547; SEQ ID NO: 548; SEQ ID NO: 549; SEQ ID NO: 550, SEQ ID NO: 551; SEQ ID NO: 552; SEQ ID NO: 553; SEQ ID NO: 554; SEQ ID NO: 555; SEQ ID NO: 556; SEQ ID NO: 557; SEQ ID NO: 558; SEQ ID NO: 559; SEQ ID NO: 560; SEQ ID NO: 561; SEQ ID NO: 562; SEQ ID NO: 563; SEQ ID NO: 564; SEQ ID NO: 565; SEQ ID NO: 566; SEQ ID NO: 567; SEQ ID NO: 568; SEQ ID NO: 569; SEQ ID NO: 570; SEQ ID NO: 571; SEQ ID NO: 572; SEQ ID NO: 573; SEQ ID NO: 574; SEQ ID NO: 575; SEQ ID NO: 576; SEQ ID NO: 577; SEQ ID NO: 578; SEQ ID NO: 579; SEQ ID NO: 580; SEQ ID NO: 581; SEQ ID NO: 582; SEQ ID NO: 583; SEQ ID NO: 584; SEQ ID NO: 585; SEQ ID NO: 586; SEQ ID NO: 587; SEQ ID NO: 588; SEQ ID NO: 589; SEQ ID NO: 590; SEQ ID NO: 591; SEQ ID NO: 592; SEQ ID NO: 593; SEQ ID NO: 594; SEQ ID NO: 595; SEQ ID NO: 596; SEQ ID NO: 597; SEQ ID NO: 598; SEQ ID NO: 599; SEQ ID NO: 600; SEQ ID NO: 601; SEQ ID NO: 602; SEQ ID NO: 603; SEQ ID NO: 604; SEQ ID NO: 605; SEQ ID NO: 606; SEQ ID NO: 607; SEQ ID NO: 608; SEQ ID NO: 609; SEQ ID NO: 610; SEQ ID NO: 611; SEQ ID NO: 612; SEQ ID NO: 613; SEQ ID NO: 614; SEQ ID NO: 615; SEQ ID NO: 616; SEQ ID NO: 617; SEQ ID NO: 618; SEQ ID NO: 619; SEQ ID NO: 620; SEQ ID NO: 621; SEQ ID NO: 622; SEQ ID NO: 623; SEQ ID NO: 624; SEQ ID NO: 625; SEQ ID NO: 626; SEQ ID NO: 627; SEQ ID NO: 628; SEQ ID NO: 629; SEQ ID NO: 630; SEQ ID NO: 631; SEQ ID NO: 632; SEQ ID NO: 633; SEQ ID NO: 634; SEQ ID NO: 635; SEQ ID NO: 636; SEQ ID NO: 637; SEQ ID NO: 638; SEQ ID NO: 639; SEQ ID NO: 640; SEQ ID NO: 641; SEQ ID NO: 642; SEQ ID NO: 643; SEQ ID NO: 644; SEQ ID NO: 645; SEQ ID NO: 646; SEQ ID NO: 647; SEQ ID NO: 648; SEQ ID NO: 649; SEQ ID NO: 650; SEQ ID NO: 651; SEQ ID NO: 652; SEQ ID NO: 653; SEQ ID NO: 654; SEQ ID NO: 655; SEQ ID NO: 656; SEQ ID NO: 657; SEQ ID NO: 658; SEQ ID NO: 659; SEQ ID NO: 660; SEQ ID NO: 661; SEQ ID NO: 662; SEQ ID NO: 663; SEQ ID NO: 664; SEQ ID NO: 665; SEQ ID NO: 666; SEQ ID NO: 667; SEQ ID NO: 668; SEQ ID NO: 669; SEQ ID NO: 670; SEQ ID NO: 671; SEQ ID NO: 672; SEQ ID NO: 673; SEQ ID NO: 674; SEQ ID NO: 675; SEQ ID NO: 676; SEQ ID NO: 677; SEQ ID NO: 678; SEQ ID NO: 679; SEQ ID NO: 680; SEQ ID NO: 681; SEQ ID NO: 682; SEQ ID NO: 683; SEQ ID NO: 684; SEQ ID NO: 685; SEQ ID NO: 686; SEQ ID NO: 687; SEQ ID NO: 688; SEQ ID NO: 689; SEQ ID NO: 690; SEQ ID NO: 691; SEQ ID NO: 692; SEQ ID NO: 693; SEQ ID NO: 694; SEQ ID NO: 695; SEQ ID NO: 696; SEQ ID NO: 697; SEQ ID NO: 698; SEQ ID NO: 699; SEQ ID NO: 700; SEQ ID NO: 701; SEQ ID NO: 702; SEQ ID NO: 703; SEQ ID NO: 704; SEQ ID NO: 705; SEQ ID NO: 706; SEQ ID NO: 707; SEQ ID NO: 708; SEQ ID NO: 709; SEQ ID NO: 710; SEQ ID NO: 711; SEQ ID NO: 712; SEQ ID NO: 713; SEQ ID NO: 714; SEQ ID NO: 715; SEQ ID NO: 716; SEQ ID NO: 717; SEQ ID NO: 718; SEQ ID NO: 719; SEQ ID NO: 720; SEQ ID NO: 721; SEQ ID NO: 722; SEQ ID NO: 723; SEQ ID NO: 724; SEQ ID NO: 725; SEQ ID NO: 726; SEQ ID NO: 727; SEQ ID NO: 728; SEQ ID NO: 729; SEQ ID NO: 730; SEQ ID NO: 731; SEQ ID NO: 732; SEQ ID NO: 733; SEQ ID NO: 734; SEQ ID NO: 735; SEQ ID NO: 736; SEQ ID NO: 737; SEQ ID NO: 738; SEQ ID NO: 739; SEQ ID NO: 740; SEQ ID NO: 741; SEQ ID NO: 742; SEQ ID NO: 743; SEQ ID NO: 744; SEQ ID NO: 745; SEQ ID NO: 746; SEQ ID NO: 747; SEQ ID NO: 748; SEQ ID NO: 749; SEQ ID NO: 750; SEQ ID NO: 751; SEQ ID NO: 752; SEQ ID NO: 753; SEQ ID NO: 754; SEQ ID NO: 755; SEQ ID NO: 756; SEQ ID NO: 757; SEQ ID NO: 758; SEQ ID NO: 759; SEQ ID NO: 760; SEQ ID NO: 761; SEQ ID NO: 762; SEQ ID NO: 763; SEQ ID NO: 764; SEQ ID NO: 765; SEQ ID NO: 766; SEQ ID NO: 767; SEQ ID NO: 768; SEQ ID NO: 769; SEQ ID NO: 770; SEQ ID NO: 771; SEQ ID NO: 772; SEQ ID NO: 773; SEQ ID NO: 774; SEQ ID NO: 775; SEQ ID NO: 776; SEQ ID NO: 777; SEQ ID NO: 778; SEQ ID NO: 779; SEQ ID NO: 780; SEQ ID NO: 781; SEQ ID NO: 782; SEQ ID NO: 783; SEQ ID NO: 784; SEQ ID NO: 785; SEQ ID NO: 786; SEQ ID NO: 787; SEQ ID NO: 788; SEQ ID NO: 789; SEQ ID NO: 790; SEQ ID NO: 791; SEQ ID NO: 792; SEQ ID NO: 793; SEQ ID NO: 794; SEQ ID NO: 795; SEQ ID NO: 796; SEQ ID NO: 797; SEQ ID NO: 798; SEQ ID NO: 799; SEQ ID NO: 800; SEQ ID NO: 801; SEQ ID NO: 802; and SEQ ID NO: 9537; SEQ ID NO: 9538; SEQ ID NO: 9539; SEQ ID NO: 9540; SEQ ID NO: 9541; SEQ ID NO: 9542; SEQ ID NO: 9543; SEQ ID NO: 9544; SEQ ID NO: 9545; SEQ ID NO: 9546; SEQ ID NO: 9547; SEQ ID NO: 9548; SEQ ID NO: 9549; SEQ ID NO: 9550; SEQ ID NO: 9551; SEQ ID NO: 9552; SEQ ID NO: 9553; and SEQ ID NO: 9554, or a complement thereof.

In another embodiment, the H. pylori cell envelope polypeptide or a fragment thereof is an H. pylori inner membrane polypeptide or a fragment thereof involved in amino acid metabolism and transport encoded by a nucleic acid having a nucleotide sequence selected from the group consisting of SEQ ID NO: 683; SEQ ID NO: 684; SEQ ID NO: 685; SEQ ID NO: 686; SEQ ID NO: 687; SEQ ID NO: 688; SEQ ID NO: 689; SEQ ID NO: 690; SEQ ID NO: 691; SEQ ID NO: 692; SEQ ID NO: 693; SEQ ID NO: 694; SEQ ID NO: 695; SEQ ID NO: 696; SEQ ID NO: 697; SEQ ID NO: 698; SEQ ID NO: 699; SEQ ID NO: 700; SEQ ID NO: 701; SEQ ID NO: 702; SEQ ID NO: 703; SEQ ID NO: 704; SEQ ID NO: 705; SEQ ID NO: 706; SEQ ID NO: 707; SEQ ID NO: 708; SEQ ID NO: 709; SEQ ID NO: 710; SEQ ID NO: 711; SEQ ID NO: 712; SEQ ID NO: 713; SEQ ID NO: 714; SEQ ID NO: 715; SEQ ID NO: 716; SEQ ID NO: 717; SEQ ID NO: 718; SEQ ID NO: 719; SEQ ID NO: 720; and SEQ ID NO: 9547 and SEQ ID NO: 9548, or a complement thereof.

In another embodiment, the H. pylori cell envelope polypeptide or a fragment thereof is an H. pylori inner membrane polypeptide or a fragment thereof involved in nucleotide, lipid, or cofactor metabolism and transport encoded by a nucleic acid having a nucleotide sequence selected from the group consisting of SEQ ID NO: 721; SEQ ID NO: 722; SEQ ID NO: 723; SEQ ID NO: 724; SEQ ID NO: 725; SEQ ID NO: 726; SEQ ID NO: 727; SEQ ID NO: 728; SEQ ID NO: 729; SEQ ID NO: 730; SEQ ID NO: 731; SEQ ID NO: 732; SEQ ID NO: 733; SEQ ID NO: 734; SEQ ID NO: 735; SEQ ID NO: 736; SEQ ID NO: 737; SEQ ID NO: 738; SEQ ID NO: 739; SEQ ID NO: 740; SEQ ID NO: 741; SEQ ID NO: 742; SEQ ID NO: 743; SEQ ID NO: 744; SEQ ID NO: 745; SEQ ID NO: 746; SEQ ID NO: 747; SEQ ID NO: 748; SEQ ID NO: 749; SEQ ID NO: 750; SEQ ID NO: 751; SEQ ID NO: 752; SEQ ID NO: 753; SEQ ID NO: 754; SEQ ID NO: 755; SEQ ID NO: 756; SEQ ID NO: 757; SEQ ID NO: 758; SEQ ID NO: 759; SEQ ID NO: 760; SEQ ID NO: 761; SEQ ID NO: 762; SEQ ID NO: 763; SEQ ID NO: 764 and SEQ ID NO: 9549, or a complement thereof.

In another embodiment, the H. pylori cell envelope polypeptide or a fragment thereof is an H. pylori inner membrane polypeptide or a fragment thereof involved in inorganic ion transport encoded by a nucleic acid having a nucleotide sequence selected from the group consisting of SEQ ID NO: 765; SEQ ID NO: 766; SEQ ID NO: 767; SEQ ID NO: 768; SEQ ID NO: 769; SEQ ID NO: 770; SEQ ID NO: 771; SEQ ID NO: 772; SEQ ID NO: 773; SEQ ID NO: 774; SEQ ID NO: 775; SEQ ID NO: 776; SEQ ID NO: 777; SEQ ID NO: 778; SEQ ID NO: 779; SEQ ID NO: 780; SEQ ID NO: 781; SEQ ID NO: 782; SEQ ID NO: 783; SEQ ID NO: 784; SEQ ID NO: 785; SEQ ID NO: 786; SEQ ID NO: 787; SEQ ID NO: 788; SEQ ID NO: 789; SEQ ID NO: 790; SEQ ID NO: 791; SEQ ID NQ: 792; SEQ ID NO: 793; SEQ ID NO: 794; SEQ ID NO: 795; SEQ ID NO: 796; SEQ ID NO: 797; SEQ ID NO: 798; SEQ ID NO: 799; and SEQ ID NO: SEQ ID NO: 9550; SEQ ID NO: 9551; SEQ ID NO: 9552; SEQ ID NO: 9553 and SEQ ID NO: 9554, or a complement thereof.

In another embodiment, the H. pylori cell envelope polypeptide or a fragment thereof is an H. pylori inner membrane polypeptide or a fragment thereof involved in carbohydrate metabolism and transport encoded by a nucleic acid having a nucleotide sequence selected from the group consisting of SEQ ID NO: 800; SEQ ID NO: 801 and SEQ ID NO: 802, or a complement thereof.

In another embodiment, the H. pylori cell envelope polypeptide or a fragment thereof is an H. pylori inner membrane polypeptide or a fragment thereof involved in outer membrane and cell wall formation encoded by a nucleic acid having a nucleotide sequence selected from the group consisting of SEQ ID NO: 803; SEQ ID NO: 804; SEQ ID NO: 805; SEQ ID NO: 806; SEQ ID NO: 807; SEQ ID NO: 808; SEQ ID NO: 809; SEQ ID NO: 810; SEQ ID NO: 811; SEQ ID NO: 812; SEQ ID NO: 813; SEQ ID NO: 814; SEQ ID NO: 815; SEQ ID NO: 816; SEQ ID NO: 817; SEQ ID NO: 818; SEQ ID NO: 819; SEQ ID NO: 820; SEQ ID NO: 821; SEQ ID NO: 822; SEQ ID NO: 823; SEQ ID NO: 824; SEQ ID NO: 825; SEQ ID NO: 826; SEQ ID NO: 827; SEQ ID NO: 828; SEQ ID NO: 829; SEQ ID NO: 830; SEQ ID NO: 831; SEQ ID NO: 832; SEQ ID NO: 833; SEQ ID NO: 834; SEQ ID NO: 835; SEQ ID NO: 836; SEQ ID NO: 837; SEQ ID NO: 838; SEQ ID NO: 839; SEQ ID NO: 840; SEQ ID NO: 841; SEQ ID NO: 842; SEQ ID NO: 843; SEQ ID NO: 844; SEQ ID NO: 845; SEQ ID NO: 846; SEQ ID NO: 847; SEQ ID NO: 848; and SEQ ID NO: 9555; SEQ ID NO: 9556; SEQ ID NO: 9557; SEQ ID NO: 9558; SEQ ID NO: 9559 and SEQ ID NO: 9560, or a complement thereof.

In another embodiment, the H. pylori cell envelope polypeptide or a fragment thereof is an H. pylori inner membrane polypeptide or a fragment thereof involved in energy conversion encoded by a nucleic acid having a nucleotide sequence selected from the group consisting of SEQ ID NO: 849; SEQ ID NO: 850; SEQ ID NO: 851; SEQ ID NO:852; SEQ ID NO: 853; SEQ ID NO: 854; SEQ ID NO: 855; SEQ ID NO: 856; SEQ ID NO: 857; SEQ ID NO: 858; SEQ ID NO: 859; SEQ ID NO: 860; SEQ ID NO: 861; SEQ ID NO: 862; SEQ ID NO: 863; SEQ ID NO: 864; SEQ ID NO: 865; SEQ ID NO: 866; SEQ ID NO: 867; SEQ ID NO: 868; SEQ ID NO: 869; SEQ ID NO: 870; SEQ ID NO: 871; SEQ ID NO: 872; SEQ ID NO: 873; SEQ ID NO: 874; SEQ ID NO: 875; SEQ ID NO: 876; SEQ ID NO: 877; SEQ ID NO: 878; SEQ ID NO: 879; SEQ ID NO: 880; SEQ ID NO: 881; SEQ ID NO: 882; SEQ ID NO: 883; SEQ ID NO: 884; SEQ ID NO: 885; SEQ ID NO: 886; SEQ ID NO: 887; SEQ ID NO: 888; SEQ ID NO: 889; SEQ ID NO: 890; SEQ ID NO: 891; SEQ ID NO: 892; SEQ ID NO: 893; SEQ ID NO: 894; SEQ ID NO: 895; SEQ ID NO: 896; SEQ ID NO: 897; SEQ ID NO: 898; SEQ ID NO: 899; SEQ ID NO: 900; SEQ ID NO: 901; SEQ ID NO: 902; SEQ ID NO: 903; SEQ ID NO: 904; SEQ ID NO: 905; SEQ ID NO: 906; SEQ ID NO: 907; SEQ ID NO: 908; SEQ ID NO: 909; SEQ ID NO: 910; SEQ ID NO: 911; SEQ ID NO: 912; SEQ ID NO: 913; SEQ ID NO: 914; SEQ ID NO: 915; SEQ ID NO: 916; SEQ ID NO: 917; SEQ ID NO: 918; SEQ ID NO: 919; SEQ ID NO: 920; SEQ ID NO: 921; SEQ ID NO: 922; SEQ ID NO: 923; SEQ ID NO: 924; SEQ ID NO: 925; SEQ ID NO: 926; SEQ ID NO: 927; SEQ ID NO: 928; SEQ ID NO: 929; SEQ ID NO: 930; SEQ ID NO: 931; SEQ ID NO: 932; SEQ ID NO: 933; SEQ ID NO: 934; SEQ ID NO: 935; SEQ ID NO: 936; SEQ ID NO: 937; SEQ ID NO: 938; SEQ ID NO: 939; SEQ ID NO: 940; SEQ ID NO: 941; SEQ ID NO: 942; SEQ ID NO: 943; SEQ ID NO: 944; SEQ ID NO: 945; SEQ ID NO: 946; SEQ ID NO: 947; SEQ ID NO: 948; SEQ ID NO: 949; SEQ ID NO: 950; SEQ ID NO: 951; SEQ ID NO: 952; SEQ ID NO: 953; SEQ ID NO: 954; SEQ ID NO: 955; SEQ ID NO: 956; SEQ ID NO: 957; SEQ ID NO: 958; SEQ ID NO: 959; SEQ ID NO: 960; SEQ ID NO: 961; SEQ ID NO: 962; SEQ ID NO: 963; SEQ ID NO: 964; SEQ ID NO: 965; SEQ ID NO: 966; SEQ ID NO: 967; SEQ ID NO: 968; SEQ ID NO: 969; SEQ ID NO: 970; SEQ ID NO: 971; SEQ ID NO: 972; SEQ ID NO: 973; SEQ ID NO: 974; SEQ ID NO: 975; SEQ ID NO: 976; SEQ ID NO: 977; SEQ ID NO: 978; SEQ ID NO: 979; SEQ ID NO: 980; SEQ ID NO: 981; SEQ ID NO: 982; SEQ ID NO: 983; SEQ ID NO: 984; SEQ ID NO: 985; SEQ ID NO: 986; SEQ ID NO: 987; SEQ ID NO: 988; SEQ ID NO: 989; SEQ ID NO: 990; SEQ ID NO: 991; SEQ ID NO: 992; SEQ ID NO: 993; SEQ ID NO: 994; SEQ ID NO: 995 and SEQ ID NO: 9561; SEQ ID NO: 9562; SEQ ID NO: 9563; SEQ ID NO: 9564 and SEQ ID NO: 9565, or a complement thereof.

In another embodiment, the H. pylori cell envelope polypeptide or a fragment thereof is an H. pylori inner membrane polypeptide or a fragment thereof involved in secretion and adhesion encoded by a nucleic acid having a nucleotide sequence selected from the group consisting of SEQ ID NO: 996; SEQ ID NO: 997; SEQ ID NO: 998; SEQ ID NO: 999; SEQ ID NO: 1000; SEQ ID NO: 1001; SEQ ID NO: 1002; SEQ ID NO: 1003; SEQ ID NO: 1004; SEQ ID NO: 1005; SEQ ID NO: 1006; SEQ ID NO: 1007 and SEQ ID NO: 9566, or a complement thereof.

In another embodiment, the H. pylori cell envelope polypeptide or a fragment thereof is an H. pylori inner membrane polypeptide or a fragment thereof involved in regulation encoded by a nucleic acid having a nucleotide sequence selected from the group consisting of SEQ ID NO: 1008; SEQ ID NO: 1009; SEQ ID NO: 1010; SEQ ID NO: 1011; SEQ ID NO: 1012; SEQ ID NO: 1013; SEQ ID NO: 1014; SEQ ID NO: 1015; SEQ ID NO: 1016; SEQ ID NO: 1017; SEQ ID NO: 1018; SEQ ID NO: 1019; SEQ ID NO: 1020; SEQ ID NO: 1021; SEQ ID NO: 1022; SEQ ID NO: 1023; SEQ ID NO: 1024; SEQ ID NO: 1025; SEQ ID NO: 1026 and SEQ ID NO: 1027, or a complement thereof.

In another embodiment, the H. pylori cell envelope polypeptide or a fragment thereof is an H. pylori inner membrane chaperone polypeptide or a fragment thereof encoded by a nucleic acid having a nucleotide sequence selected from the group consisting of SEQ ID NO: 1028; SEQ ID NO: 1029; SEQ ID NO: 1030; SEQ ID NO: 1031 and SEQ ID NO: 9567, or a complement thereof.

In another embodiment, the H. pylori cell envelope polypeptide or a fragment thereof is an H. pylori inner membrane polypeptide or a fragment thereof involved in cell division encoded by a nucleic acid having a nucleotide sequence selected from the group consisting of SEQ ID NO: 1032; SEQ ID NO: 1033; SEQ ID NO: 1034; SEQ ID NO: 1035; SEQ ID NO: 1036; SEQ ID NO: 1037; SEQ ID NO: 1038; SEQ ID NO: 1039; SEQ ID NO: 1040; SEQ ID NO: 1041; SEQ ID NO: 1042; SEQ ID NO: 1043; SEQ ID NO: 1044; SEQ ID NO: 1045; SEQ ID NO: 1046; SEQ ID NO: 1047; SEQ ID NO: 1048; SEQ ID NO: 1049; SEQ ID NO: 1050; SEQ ID NO: 1051; SEQ ID NO: 1052; and SEQ ID NO: 9568 and SEQ ID NO: 9569, or a complement thereof.

In another embodiment, the H. pylori cell envelope polypeptide or a fragment thereof is an H. pylori inner membrane polypeptide or a fragment thereof involved in motility encoded by a nucleic acid having a nucleotide sequence selected from the group consisting of SEQ ID NO: 1053 and SEQ ID NO: 1054, or a complement thereof.

Particularly preferred is an isolated nucleic acid having a nucleotide sequence encoding an H. pylori cytoplasmic polypeptide or a fragment thereof, the nucleic acid selected from the group consisting of SEQ ID NO: 1576; SEQ ID NO: 1577; SEQ ID NO: 1578; SEQ ID NO: 1579; SEQ ID NO: 1580; SEQ ID NO: 1581; SEQ ID NO: 1582; SEQ ID NO: 1583; SEQ ID NO: 1584; SEQ ID NO: 1585; SEQ ID NO: 1586; SEQ ID NO: 1587; SEQ ID NO: 1588; SEQ ID NO: 1589; SEQ ID NO: 1590; SEQ ID NO: 1591; SEQ ID NO: 1592; SEQ ID NO: 1593; SEQ ID NO: 1594; SEQ ID NO: 1595; SEQ ID NO: 1596; SEQ ID NO: 1597; SEQ ID NO: 1598; SEQ ID NO: 1599; SEQ ID NO: 1600; SEQ ID NO: 1601; SEQ ID NO: 1602; SEQ ID NO: 1603; SEQ ID NO: 1604; SEQ ID NO: 1605; SEQ ID NO: 1606; SEQ ID NO: 1607; SEQ ID NO: 1608; SEQ ID NO: 1609; SEQ ID NO: 1610; SEQ ID NO: 1611; SEQ ID NO: 1612; SEQ ID NO: 1613; SEQ ID NO: 1614; SEQ ID NO: 1615; SEQ ID NO: 1616; SEQ ID NO: 1617; SEQ ID NO: 1618; SEQ ID NO: 1619; SEQ ID NO: 1620; SEQ ID NO: 1621; SEQ ID NO: 1622; SEQ ID NO: 1623; SEQ ID NO: 1624; SEQ ID NO: 1625; SEQ ID NO: 1626; SEQ ID NO: 1627; SEQ ID NO: 1628; SEQ ID NO: 1629; SEQ ID NO: 1630; SEQ ID NO: 1631; SEQ ID NO: 1632; SEQ ID NO: 1633; SEQ ID NO: 1634; SEQ ID NO: 1635; SEQ ID NO: 1636; SEQ ID NO: 1637; SEQ ID NO: 1638; SEQ ID NO: 1639; SEQ ID NO: 1640; SEQ ID NO: 1641; SEQ ID NO: 1642; SEQ ID NO: 1643; SEQ ID NO: 1644; SEQ ID NO: 1645; SEQ ID NO: 1646; SEQ ID NO: 1647; SEQ ID NO: 1648; SEQ ID NO: 1649; SEQ ID NO: 1650; SEQ ID NO: 1651; SEQ ID NO: 1652; SEQ ID NO: 1653; SEQ ID NO: 1654; SEQ ID NO: 1655; SEQ ID NO: 1656; SEQ ID NO: 1657; SEQ ID NO: 1658; SEQ ID NO: 1659; SEQ ID NO: 1660; SEQ ID NO: 1661; SEQ ID NO: 1662; SEQ ID NO: 1663; SEQ ID NO: 1664; SEQ ID NO: 1665; SEQ ID NO: 1666; SEQ ID NO: 1667; SEQ ID NO: 1668; SEQ ID NO: 1669; SEQ ID NO: 1670; SEQ ID NO: 1671; SEQ ID NO: 1672; SEQ ID NO: 1673; SEQ ID NO: 1674; SEQ ID NO: 1675; SEQ ID NO: 1676; SEQ ID NO: 1677; SEQ ID NO: 1678; SEQ ID NO: 1679; SEQ ID NO: 1680; SEQ ID NO: 1681; SEQ ID NO: 1682; SEQ ID NO: 1683; SEQ ID NO: 1684; SEQ ID NO: 1685; SEQ ID NO: 1686; SEQ ID NO: 1687; SEQ ID NO: 1688; SEQ ID NO: 1689; SEQ ID NO: 1690; SEQ ID NO: 1691; SEQ ID NO: 1692; SEQ ID NO: 1693; SEQ ID NO: 1694; SEQ ID NO: 1695; SEQ ID NO: 1696; SEQ ID NO: 1697; SEQ ID NO: 1698; SEQ ID NO: 1699; SEQ ID NO: 1700; SEQ ID NO: 1701; SEQ ID NO: 1702; SEQ ID NO: 1703; SEQ ID NO: 1704; SEQ ID NO: 1705; SEQ ID NO: 1706; SEQ ID NO: 1707; SEQ ID NO: 1708; SEQ ID NO: 1709; SEQ ID NO: 1710; SEQ ID NO: 1711; SEQ ID NO: 1712; SEQ ID NO: 1713; SEQ ID NO: 1714; SEQ ID NO: 1715; SEQ ID NO: 1716; SEQ ID NO: 1717; SEQ ID NO: 1718; SEQ ID NO: 1719; SEQ ID NO: 1720; SEQ ID NO: 1721; SEQ ID NO: 1722; SEQ ID NO: 1723; SEQ ID NO: 1724; SEQ ID NO: 1725; SEQ ID NO: 1726; SEQ ID NO: 1727; SEQ ID NO: 1728; SEQ ID NO: 1729; SEQ ID NO: 1730; SEQ ID NO: 1731; SEQ ID NO: 1732; SEQ ID NO: 1733; SEQ ID NO: 1734; SEQ ID NO: 1735; SEQ ID NO: 1736; SEQ ID NO: 1737; SEQ ID NO: 1738; SEQ ID NO: 1739; SEQ ID NO: 1740; SEQ ID NO: 1741; SEQ ID NO: 1742; SEQ ID NO: 1743; SEQ ID NO: 1744; SEQ ID NO: 1745; SEQ ID NO: 1746; SEQ ID NO: 1747; SEQ ID NO: 1748; SEQ ID NO: 1749; SEQ ID NO: 1750; SEQ ID NO: 1751; SEQ ID NO: 1752; SEQ ID NO: 1753; SEQ ID NO: 1754; SEQ ID NO: 1755; SEQ ID NO: 1756; SEQ ID NO: 1757; SEQ ID NO: 1758; SEQ ID NO: 1759; SEQ ID NO: 1760; SEQ ID NO: 1761; SEQ ID NO: 1762; SEQ ID NO: 1763; SEQ ID NO: 1764; SEQ ID NO: 1765; SEQ ID NO: 1766; SEQ ID NO: 1767; SEQ ID NO: 1768; SEQ ID NO: 1769; SEQ ID NO: 1770; SEQ ID NO: 1771; SEQ ID NO: 1772; SEQ ID NO: 1773; SEQ ID NO: 1774; SEQ ID NO: 1775; SEQ ID NO: 1776; SEQ ID NO: 1777; SEQ ID NO: 1778; SEQ ID NO: 1779; SEQ ID NO: 1780; SEQ ID NO: 1781; SEQ ID NO: 1782; SEQ ID NO: 1783; SEQ ID NO: 1784; SEQ ID NO: 1785; SEQ ID NO: 1786; SEQ ID NO: 1787; SEQ ID NO: 1788; SEQ ID NO: 1789; SEQ ID NO: 1790; SEQ ID NO: 1791; SEQ ID NO: 1792; SEQ ID NO: 1793; SEQ ID NO: 1794; SEQ ID NO: 1795; SEQ ID NO: 1796; SEQ ID NO: 1797; SEQ ID NO: 1798; SEQ ID NO: 1799; SEQ ID NO: 1800; SEQ ID NO: 1801; SEQ ID NO: 1802; SEQ ID NO: 1803; SEQ ID NO: 1804; SEQ ID NO: 1805; SEQ ID NO: 1806; SEQ ID NO: 1807; SEQ ID NO: 1808; SEQ ID NO: 1809; SEQ ID NO: 1810; SEQ ID NO: 1811; SEQ ID NO: 1812; SEQ ID NO: 1813; SEQ ID NO: 1814; SEQ ID NO: 1815; SEQ ID NO: 1816; SEQ ID NO: 1817; SEQ ID NO: 1818; SEQ ID NO: 1819; SEQ ID NO: 1820; SEQ ID NO: 1821; SEQ ID NO: 1822; SEQ ID NO: 1823; SEQ ID NO: 1824; SEQ ID NO: 1825; SEQ ID NO: 1826; SEQ ID NO: 1827; SEQ ID NO: 1828; SEQ ID NO: 1829; SEQ ID NO: 1830; SEQ ID NO: 1831; SEQ ID NO: 1832; SEQ ID NO: 1833; SEQ ID NO: 1834; SEQ ID NO: 1835; SEQ ID NO: 1836; SEQ ID NO: 1837; SEQ ID NO: 1838; SEQ ID NO: 1839; SEQ ID NO: 1840; SEQ ID NO: 1841; SEQ ID NO: 1842; SEQ ID NO: 1843; SEQ ID NO: 1844; SEQ ID NO: 1845; SEQ ID NO: 1846; SEQ ID NO: 1847; SEQ ID NO: 1848; SEQ ID NO: 1849; SEQ ID NO: 1850; SEQ ID NO: 1851; SEQ ID NO: 1852; SEQ ID NO: 1853; SEQ ID NO: 1854; SEQ ID NO: 1855; SEQ ID NO: 1856; SEQ ID NO: 1857; SEQ ID NO: 1858; SEQ ID NO: 1859; SEQ ID NO: 1860; SEQ ID NO: 1861; SEQ ID NO: 1862; SEQ ID NO: 1863; SEQ ID NO: 1864; SEQ ID NO: 1865; SEQ ID NO: 1866; SEQ ID NO: 1867; SEQ ID NO: 1868; SEQ ID NO: 1869; SEQ ID NO: 1870; SEQ ID NO: 1871; SEQ ID NO: 1872; SEQ ID NO: 1873; SEQ ID NO: 1874; SEQ ID NO: 1875; SEQ ID NO: 1876; SEQ ID NO: 1877; SEQ ID NO: 1878; SEQ ID NO: 1879; SEQ ID NO: 1880; SEQ ID NO: 1881; SEQ ID NO: 1882; SEQ ID NO: 1883; SEQ ID NO: 1884; SEQ ID NO: 1885; SEQ ID NO: 1886; SEQ ID NO: 1887; SEQ ID NO: 1888; SEQ ID NO: 1889; SEQ ID NO: 1890; SEQ ID NO: 1891; SEQ ID NO: 1892; SEQ ID NO: 1893; SEQ ID NO: 1894; SEQ ID NO: 1895; SEQ ID NO: 1896; SEQ ID NO: 1897; SEQ ID NO: 1898; SEQ ID NO: 1899; SEQ ID NO: 1900; SEQ ID NO: 1901; SEQ ID NO: 1902; SEQ ID NO: 1903; SEQ ID NO: 1904; SEQ ID NO: 1905; SEQ ID NO: 1906; SEQ ID NO: 1907; SEQ ID NO: 1908; SEQ ID NO: 1909; SEQ ID NO: 1910; SEQ ID NO: 1911; SEQ ID NO: 1912; SEQ ID NO: 1913; SEQ ID NO: 1914; SEQ ID NO: 1915; SEQ ID NO: 1916; SEQ ID NO: 1917; SEQ ID NO: 1918; SEQ ID NO: 1919; SEQ ID NO: 1920; SEQ ID NO: 1921; SEQ ID NO: 1922; SEQ ID NO: 1923; SEQ ID NO: 1924; SEQ ID NO: 1925; SEQ ID NO: 1926; SEQ ID NO: 1927; SEQ ID NO: 1928; SEQ ID NO. 1929; SEQ ID NO: 1930; SEQ ID NO: 1931; SEQ ID NO: 1932; SEQ ID NO: 1933; SEQ ID NO: 1934; SEQ ID NO: 1935; SEQ ID NO: 1936; SEQ ID NO: 1937; SEQ ID NO: 1938; SEQ ID NO: 1939; SEQ ID NO: 1940; SEQ ID NO: 1941; SEQ ID NO: 1942; SEQ ID NO: 1943; SEQ ID NO: 1944; SEQ ID NO: 1945; SEQ ID NO: 1946; SEQ ID NO: 1947; SEQ ID NO: 1948; SEQ ID NO: 1949; SEQ ID NO: 1950; SEQ ID NO: 1951; SEQ ID NO: 1952; SEQ ID NO: 1953; SEQ ID NO: 1954; SEQ ID NO: 1955; SEQ ID NO: 1956; SEQ ID NO: 1957; SEQ ID NO: 1958; SEQ ID NO: 1959; SEQ ID NO: 1960; SEQ ID NO: 1961; SEQ ID NO: 1962; SEQ ID NO: 1963; SEQ ID NO: 1964; SEQ ID NO: 1965; SEQ ID NO: 1966; SEQ ID NO: 1967; SEQ ID NO: 1968; SEQ ID NO: 1969; SEQ ID NO: 1970; SEQ ID NO: 1971; SEQ ID NO: 1972; SEQ ID NO: 1973; SEQ ID NO: 1974; SEQ ID NO: 1975; SEQ ID NO: 1976; SEQ ID NO: 1977; SEQ ID NO: 1978; SEQ ID NO: 1979; SEQ ID NO: 1980; SEQ ID NO: 1981; SEQ ID NO: 1982; SEQ ID NO: 1983; SEQ ID NO: 1984; SEQ ID NO: 1985; SEQ ID NO: 1986; SEQ ID NO: 1987; SEQ ID NO: 1988; SEQ ID NO: 1989; SEQ ID NO: 1990; SEQ ID NO: 1991; SEQ ID NO: 1992; SEQ ID NO: 1993; SEQ ID NO: 1994; SEQ ID NO: 1995; SEQ ID NO: 1996; SEQ ID NO: 1997; SEQ ID NO: 1998; SEQ ID NO: 1999; SEQ ID NO: 2000; SEQ ID NO: 2001; SEQ ID NO: 2002; SEQ ID NO: 2003; SEQ ID NO: 2004; SEQ ID NO: 2005; SEQ ID NO: 2006; SEQ ID NO: 2007; SEQ ID NO: 2008; SEQ ID NO: 2009; SEQ ID NO: 2010; SEQ ID NO: 2011; SEQ ID NO: 2012; SEQ ID NO: 2013; SEQ ID NO: 2014; SEQ ID NO: 2015; SEQ ID NO: 2016; SEQ ID NO: 2017; SEQ ID NO: 2018; SEQ ID NO: 2019; SEQ ID NO: 2020; SEQ ID NO: 2021; SEQ ID NO: 2022; SEQ ID NO: 2023; SEQ ID NO: 2024; SEQ ID NO: 2025; SEQ ID NO: 2026; SEQ ID NO: 2027; SEQ ID NO: 2028; SEQ ID NO: 2029; SEQ ID NO: 2030; SEQ ID NO: 2031; SEQ ID NO: 2032; SEQ ID NO: 2033; SEQ ID NO: 2034; SEQ ID NO: 2035; SEQ ID NO: 2036; SEQ ID NO: 2037; SEQ ID NO: 2038; SEQ ID NO: 2039; SEQ ID NO: 2040; SEQ ID NO: 2041; SEQ ID NO: 2042; SEQ ID NO: 2043; SEQ ID NO: 2044; SEQ ID NO: 2045; SEQ ID NO: 2046; SEQ ID NO: 2047; SEQ ID NO: 2048; SEQ ID NO: 2049; SEQ ID NO: 2050; SEQ ID NO: 2051; SEQ ID NO: 2052; SEQ ID NO: 2053; SEQ ID NO: 2054; SEQ ID NO: 2055; SEQ ID NO: 2056; SEQ ID NO: 2057; SEQ ID NO: 2058; SEQ ID NO: 2059; SEQ ID NO: 2060; SEQ ID NO: 2061; SEQ ID NO: 2062; SEQ ID NO: 2063; SEQ ID NO: 2064; SEQ ID NO: 2065; SEQ ID NO: 2066; SEQ ID NO: 2067; SEQ ID NO: 2068; SEQ ID NO: 2069; SEQ ID NO: 2070; SEQ ID NO: 2071; SEQ ID NO: 2072; SEQ ID NO: 2073; SEQ ID NO: 2074; SEQ ID NO: 2075; SEQ ID NO: 2076; SEQ ID NO: 2077; SEQ ID NO: 2078; SEQ ID NO: 2079; SEQ ID NO: 2080; SEQ ID NO: 2081; SEQ ID NO: 2082; SEQ ID NO: 2083; SEQ ID NO: 2084; SEQ ID NO: 2085; SEQ ID NO: 2086; SEQ ID NO: 2087; SEQ ID NO: 2088; SEQ ID NO: 2089; SEQ ID NO: 2090; SEQ ID NO: 2091; SEQ ID NO: 2092; SEQ ID NO: 2093; SEQ ID NO: 2094; SEQ ID NO: 2095; SEQ ID NO: 2096; SEQ ID NO: 2097; SEQ ID NO: 2098; SEQ ID NO: 2099; SEQ ID NO: 2100; SEQ ID NO: 2101; SEQ ID NO: 2102; SEQ ID NO: 2103; SEQ ID NO: 2104; SEQ ID NO: 2105; SEQ ID NO: 2106; SEQ ID NO: 2107; SEQ ID NO: 2108; SEQ ID NO: 2109; SEQ ID NO: 2110; SEQ ID NO: 2111; SEQ ID NO: 2112; SEQ ID NO: 2113; SEQ ID NO: 2114; SEQ ID NO: 2115; SEQ ID NO: 2116; SEQ ID NO: 2117; SEQ ID NO: 2118; SEQ ID NO: 2119; SEQ ID NO: 2120; SEQ ID NO: 2121; SEQ ID NO: 2122; SEQ ID NO: 2123; SEQ ID NO: 2124; SEQ ID NO: 2125; SEQ ID NO: 2126; SEQ ID NO: 2127; SEQ ID NO: 2128; SEQ ID NO: 2129; SEQ ID NO: 2130; SEQ ID NO: 2131; SEQ ID NO: 2132; SEQ ID NO: 2133; SEQ ID NO: 2134; SEQ ID NO: 2135; SEQ ID NO: 2136; SEQ ID NO: 2137; SEQ ID NO: 2138; SEQ ID NO: 2139; SEQ ID NO: 2140; SEQ ID NO: 2141; SEQ ID NO: 2142; SEQ ID NO: 2143; SEQ ID NO: 2144; SEQ ID NO: 2145; SEQ ID NO: 2146; SEQ ID NO: 2147; SEQ ID NO: 2148; SEQ ID NO: 2149; SEQ ID NO: 2150; SEQ ID NO: 2151; SEQ ID NO: 2152; SEQ ID NO: 2153; SEQ ID NO: 2154; SEQ ID NO: 2155; SEQ ID NO: 2156; SEQ ID NO: 2157; SEQ ID NO: 2158; SEQ ID NO: 2159; SEQ ID NO: 2160; SEQ ID NO: 2161; SEQ ID NO: 2162; SEQ ID NO: 2163; SEQ ID NO: 2164; SEQ ID NO: 2165; SEQ ID NO: 2166; SEQ ID NO: 2167; SEQ ID NO: 2168; SEQ ID NO: 2169; SEQ ID NO: 2170; SEQ ID NO: 2171; SEQ ID NO: 2172; SEQ ID NO: 2173; SEQ ID NO: 2174; SEQ ID NO: 2175; SEQ ID NO: 2176; SEQ ID NO: 2177; SEQ ID NO: 2178; SEQ ID NO: 2179; SEQ ID NO: 2180; SEQ ID NO: 2181; SEQ ID NO: 2182; SEQ ID NO: 2183; SEQ ID NO: 2184; SEQ ID NO: 2185; SEQ ID NO: 2186; SEQ ID NO: 2187; SEQ ID NO: 2188; SEQ ID NO: 2189; SEQ ID NO: 2190; SEQ ID NO: 2191; SEQ ID NO: 2192; SEQ ID NO: 2193; SEQ ID NO: 2194; SEQ ID NO: 2195; SEQ ID NO: 2196; SEQ ID NO: 2197; SEQ ID NO: 2198; SEQ ID NO: 2199; SEQ ID NO: 2200; SEQ ID NO: 2201; SEQ ID NO: 2202; SEQ ID NO: 2203; SEQ ID NO: 2204; SEQ ID NO: 2205; SEQ ID NO: 2206; SEQ ID NO: 2207; SEQ ID NO: 2208; SEQ ID NO: 2209; SEQ ID NO: 2210; SEQ ID NO: 2211; SEQ ID NO: 2212; SEQ ID NO: 2213; SEQ ID NO: 2214; SEQ ID NO: 2215; SEQ ID NO: 2216; SEQ ID NO: 2217; SEQ ID NO: 2218; SEQ ID NO: 2219; SEQ ID NO: 2220; SEQ ID NO: 2221; SEQ ID NO: 2222; SEQ ID NO: 2223; SEQ ID NO: 2224; SEQ ID NO: 2225; SEQ ID NO: 2226; SEQ ID NO: 2227; SEQ ID NO: 2228; SEQ ID NO: 2229; SEQ ID NO: 2230; SEQ ID NO: 2231; SEQ ID NO: 2232; SEQ ID NO: 2233; SEQ ID NO: 2234; SEQ ID NO: 2235; SEQ ID NO: 2236; SEQ ID NO: 2237; SEQ ID NO: 2238; SEQ ID NO: 2239; SEQ ID NO: 2240; SEQ ID NO: 2241; SEQ ID NO: 2242; SEQ ID NO: 2243; SEQ ID NO: 2244; SEQ ID NO: 2245; SEQ ID NO: 2246; SEQ ID NO: 2247; SEQ ID NO: 2248; SEQ ID NO: 2249; SEQ ID NO: 2250; SEQ ID NO: 2251; SEQ ID NO: 2252; SEQ ID NO: 2253; SEQ ID NO: 2254; SEQ ID NO: 2255; SEQ ID NO: 2256; SEQ ID NO: 2257; SEQ ID NO: 2258; SEQ ID NO: 2259; SEQ ID NO: 2260; SEQ ID NO: 2261; SEQ ID NO: 2262; SEQ ID NO: 2263; SEQ ID NO: 2264; SEQ ID NO: 2265; SEQ ID NO: 2266; SEQ ID NO: 2267; SEQ ID NO: 2268; SEQ ID NO: 2269; SEQ ID NO: 2270; SEQ ID NO: 2271; SEQ ID NO: 2272; SEQ ID NO: 2273; SEQ ID NO: 2274; SEQ ID NO: 2275; SEQ ID NO: 2276; SEQ ID NO: 2277; SEQ ID NO: 2278; SEQ ID NO: 2279; SEQ ID NO: 2280; SEQ ID NO: 2281; SEQ ID NO: 2282; SEQ ID NO: 2283; SEQ ID NO: 2284; SEQ ID NO: 2285; SEQ ID NO: 2286; SEQ ID NO: 2287; SEQ ID NO: 2288; SEQ ID NO: 2289; SEQ ID NO: 2290; SEQ ID NO: 2291; SEQ ID NO: 2292; SEQ ID NO:2293; SEQ ID NO:2294; SEQ ID NO:2295; SEQ ID NO:2296; SEQ ID NO: 2297; SEQ ID NO: 2298; SEQ ID NO: 2299; SEQ ID NO: 2300; SEQ ID NO: 2301; SEQ ID NO: 2302; SEQ ID NO: 2303; SEQ ID NO: 2304; SEQ ID NO: 2305; SEQ ID NO: 2306; SEQ ID NO: 2307; SEQ ID NO: 2308; SEQ ID NO: 2309; SEQ ID NO: 2310; SEQ ID NO: 2311; SEQ ID NO: 2312; SEQ ID NO: 2313; SEQ ID NO: 2314; SEQ ID NO: 2315; SEQ ID NO: 2316; SEQ ID NO: 2317; SEQ ID NO: 2318; SEQ ID NO: 2319; SEQ ID NO: 2320; SEQ ID NO: 2321; SEQ ID NO: 2322; SEQ ID NO: 2323; SEQ ID NO: 2324; SEQ ID NO: 2325; SEQ ID NO: 2326; SEQ ID NO: 2327; SEQ ID NO: 2328; SEQ ID NO: 2329; SEQ ID NO: 2330; SEQ ID NO: 2331; SEQ ID NO: 2332; SEQ ID NO: 2333; SEQ ID NO: 2334; SEQ ID NO: 2335; SEQ ID NO: 2336; SEQ ID NO: 2337; SEQ ID NO: 2338; SEQ ID NO: 2339; SEQ ID NO: 2340; SEQ ID NO: 2341; SEQ ID NO: 2342; SEQ ID NO: 2343; SEQ ID NO: 2344; SEQ ID NO: 2345; SEQ ID NO: 2346; SEQ ID NO: 2347; SEQ ID NO: 2348; SEQ ID NO: 2349; SEQ ID NO: 2350; SEQ ID NO: 2351; SEQ ID NO: 2352; SEQ ID NO: 2353; SEQ ID NO: 2354; SEQ ID NO: 2355; SEQ ID NO: 2356; SEQ ID NO: 2357; SEQ ID NO: 2358; SEQ ID NO: 2359; SEQ ID NO: 2360; SEQ ID NO: 2361; SEQ ID NO: 2362; SEQ ID NO: 2363; SEQ ID NO: 2364; SEQ ID NO: 2365; SEQ ID NO: 2366; SEQ ID NO: 2367; SEQ ID NO: 2368; SEQ ID NO: 2369; SEQ ID NO: 2370; SEQ ID NO: 2371; SEQ ID NO: 2372; SEQ ID NO: 2373; SEQ ID NO: 2374; SEQ ID NO: 2375; SEQ ID NO: 2376; SEQ ID NO: 2377; SEQ ID NO: 2378; SEQ ID NO: 2379; SEQ ID NO: 2380; SEQ ID NO: 2381; SEQ ID NO: 2382; SEQ ID NO: 2383; SEQ ID NO: 2384; SEQ ID NO: 2385; SEQ ID NO: 2386; SEQ ID NO: 2387; SEQ ID NO: 2388; SEQ ID NO: 2389; SEQ ID NO: 2390; SEQ ID NO: 2391; SEQ ID NO: 2392; SEQ ID NO: 2393; SEQ ID NO: 2394; SEQ ID NO: 2395; SEQ ID NO: 2396; SEQ ID NO: 2397; SEQ ID NO: 2398; SEQ ID NO: 2399; SEQ ID NO: 2400; SEQ ID NO: 2401; SEQ ID NO: 2402; SEQ ID NO: 2403; SEQ ID NO: 2404; SEQ ID NO: 2405; SEQ ID NO: 2406; SEQ ID NO: 2407; SEQ ID NO: 2408; SEQ ID NO: 2409; SEQ ID NO: 2410; SEQ ID NO: 2411; SEQ ID NO: 2412; SEQ ID NO: 2413; SEQ ID NO: 2414; SEQ ID NO: 2415; SEQ ID NO: 2416; SEQ ID NO: 2417; SEQ ID NO: 2418; SEQ ID NO: 2419; SEQ ID NO: 2420; SEQ ID NO: 2421; SEQ ID NO: 2422; SEQ ID NO: 2423; SEQ ID NO: 2424; SEQ ID NO: 2425; SEQ ID NO: 2426; SEQ ID NO: 2427; SEQ ID NO: 2428; SEQ ID NO: 2429; SEQ ID NO: 2430; SEQ ID NO: 2431; SEQ ID NO: 2432; SEQ ID NO: 2433; SEQ ID NO: 2434; SEQ ID NO: 2435; SEQ ID NO: 2436; SEQ ID NO: 2437; SEQ ID NO: 2438; SEQ ID NO: 2439; SEQ ID NO: 2440; SEQ ID NO: 2441; SEQ ID NO: 2442; SEQ ID NO: 2443; SEQ ID NO: 2444; SEQ ID NO: 2445; SEQ ID NO: 2446; SEQ ID NO: 2447; SEQ ID NO: 2448; SEQ ID NO: 2449; SEQ ID NO: 2450; SEQ ID NO: 2451; SEQ ID NO: 2452; SEQ ID NO: 2453; SEQ ID NO: 2454; SEQ ID NO: 2455; SEQ ID NO: 2456; SEQ ID NO: 2457; SEQ ID NO: 2458; SEQ ID NO: 2459; SEQ ID NO: 2460; SEQ ID NO: 2461; SEQ ID NO: 2462; SEQ ID NO: 2463; SEQ ID NO: 2464; SEQ ID NO: 2465; SEQ ID NO: 2466; SEQ ID NO: 2467; SEQ ID NO: 2468; SEQ ID NO: 2469; SEQ ID NO: 2470; SEQ ID NO: 2471; SEQ ID NO: 2472; SEQ ID NO: 2473; SEQ ID NO: 2474; SEQ ID NO: 2475; SEQ ID NO: 2476; SEQ ID NO: 2477; SEQ ID NO: 2478; SEQ ID NO: 2479; SEQ ID NO: 2480; SEQ ID NO: 2481; SEQ ID NO: 2482; SEQ ID NO: 2483; SEQ ID NO: 2484; SEQ ID NO: 2485; SEQ ID NO: 2486; SEQ ID NO: 2487; SEQ ID NO: 2488; SEQ ID NO: 2489; SEQ ID NO: 2490; SEQ ID NO: 2491; SEQ ID NO: 2492; SEQ ID NO: 2493; SEQ ID NO: 2494; SEQ ID NO: 2495; SEQ ID NO: 2496; SEQ ID NO: 2497; SEQ ID NO: 2498; SEQ ID NO: 2499; SEQ ID NO: 2500; SEQ ID NO: 2501; SEQ ID NO: 2502; SEQ ID NO: 2503; SEQ ID NO: 2504; SEQ ID NO: 2505; SEQ ID NO: 2506; SEQ ID NO: 2507; SEQ ID NO: 2508; SEQ ID NO: 2509; SEQ ID NO: 2510; SEQ ID NO: 2511; SEQ ID NO: 2512; SEQ ID NO: 2513; SEQ ID NO: 2514; SEQ ID NO: 2515; SEQ ID NO: 2516; SEQ ID NO: 2517; SEQ ID NO: 2518; SEQ ID NO: 2519; SEQ ID NO: 2520; SEQ ID NO: 2521; SEQ ID NO: 2522; SEQ ID NO: 2523; SEQ ID NO: 2524; SEQ ID NO: 2525; SEQ ID NO: 2526; SEQ ID NO: 2527; SEQ ID NO: 2528; SEQ ID NO: 2529; SEQ ID NO: 2530; SEQ ID NO: 2531; SEQ ID NO: 2532; SEQ ID NO: 2533; SEQ ID NO: 2534; SEQ ID NO: 2535; SEQ ID NO: 2536; SEQ ID NO: 2537; SEQ ID NO: 2538; SEQ ID NO: 2539; SEQ ID NO: 2540; SEQ ID NO: 2541; SEQ ID NO: 2542; SEQ ID NO: 2543; SEQ ID NO: 2544; SEQ ID NO: 2545; SEQ ID NO: 2546; SEQ ID NO: 2547; SEQ ID NO: 2548; SEQ ID NO: 2549; SEQ ID NO: 2550; SEQ ID NO: 2551; SEQ ID NO: 2552; SEQ ID NO: 2553; SEQ ID NO: 2554; SEQ ID NO: 2555; SEQ ID NO: 2556; SEQ ID NO: 2557; SEQ ID NO: 2558; SEQ ID NO: 2559; SEQ ID NO: 2560; SEQ ID NO: 2561; SEQ ID NO: 2562; SEQ ID NO: 2563; SEQ ID NO: 2564; SEQ ID NO: 2565; SEQ ID NO: 2566; SEQ ID NO: 2567; SEQ ID NO: 2568; SEQ ID NO: 2569; SEQ ID NO: 2570; SEQ ID NO: 2571; SEQ ID NO: 2572; SEQ ID NO: 2573; SEQ ID NO: 2574; SEQ ID NO: 2575; SEQ ID NO: 2576; SEQ ID NO: 2577; SEQ ID NO: 2578; SEQ ID NO: 2579; SEQ ID NO: 2580; SEQ ID NO: 2581; SEQ ID NO: 2582; SEQ ID NO: 2583; SEQ ID NO: 2584; SEQ ID NO: 2585; SEQ ID NO: 2586; SEQ ID NO: 2587; SEQ ID NO: 2588; SEQ ID NO: 2589; SEQ ID NO: 2590; SEQ ID NO: 2591; SEQ ID NO: 2592; SEQ ID NO: 2593; SEQ ID NO: 2594; SEQ ID NO: 2595; SEQ ID NO: 2596; SEQ ID NO: 2597; SEQ ID NO: 2598; SEQ ID NO: 2599; SEQ ID NO: 2600; SEQ ID NO: 2601; SEQ ID NO: 2602; SEQ ID NO: 2603; SEQ ID NO: 2604; SEQ ID NO: 2605; SEQ ID NO: 2606; SEQ ID NO: 2607; SEQ ID NO: 2608; SEQ ID NO: 2609; SEQ ID NO: 2610; SEQ ID NO: 2611; SEQ ID NO: 2612; SEQ ID NO: 2613; SEQ ID NO: 2614; SEQ ID NO: 2615; SEQ ID NO: 2616; SEQ ID NO: 2617; SEQ ID NO: 2618; SEQ ID NO: 2619; SEQ ID NO: 2620; SEQ ID NO: 2621; SEQ ID NO: 2622; SEQ ID NO: 2623; SEQ ID NO: 2624; SEQ ID NO: 2625; SEQ ID NO: 2626; SEQ ID NO: 2627; SEQ ID NO: 2628; SEQ ID NO: 2629; SEQ ID NO: 2630; SEQ ID NO: 2631; SEQ ID NO: 2632; SEQ ID NO: 2633; SEQ ID NO: 2634; SEQ ID NO: 2635; SEQ ID NO: 2636; SEQ ID NO: 2637; SEQ ID NC: 638; SEQ ID NO: 2639; SEQ ID NO: 2640; SEQ ID NO: 2641; SEQ ID NO: 2642; SEQ ID NO: 2643; SEQ ID NO: 2644; SEQ ID NO: 2645; SEQ ID NO: 2646; SEQ ID NO: 2647; SEQ ID NO: 2648; SEQ ID NO: 2649; SEQ ID NO: 2650; SEQ ID NO: 2651; SEQ ID NO: 2652; SEQ ID NO: 2653; SEQ ID NO: 2654; SEQ ID NO: 2655; SEQ ID NO: 2656; SEQ ID NO: 2657; SEQ ID NO: 2658; SEQ ID NO: 2659; SEQ ID NO: 2660; SEQ ID NO: 2661; SEQ ID NO: 2662; SEQ ID NO: 2663; SEQ ID NO: 2664; SEQ ID NO: 2665; SEQ ID NO: 2666; SEQ ID NO: 2667; SEQ ID NO: 2668; SEQ ID NO: 2669; SEQ ID NO: 2670; SEQ ID NO: 2671; SEQ ID NO: 2672; SEQ ID NO: 2673; SEQ ID NO: 2674; SEQ ID NO: 2675; SEQ ID NO: 2676; SEQ ID NO: 2677; SEQ ID NO: 2678; SEQ ID NO: 2679; SEQ ID NO: 2680; SEQ ID NO: 2681; SEQ ID NO: 2682; SEQ ID NO: 2683; SEQ ID NO: 2684; SEQ ID NO: 2685; SEQ ID NO: 2686; SEQ ID NO: 2687; SEQ ID NO: 2688; SEQ ID NO: 2689; SEQ ID NO: 2690; SEQ ID NO: 2691; SEQ ID NO: 2692; SEQ ID NO: 2693; SEQ ID NO: 2694; SEQ ID NO: 2695; SEQ ID NO: 2696; SEQ ID NO: 2697; SEQ ID NO: 2698; SEQ ID NO: 2699; SEQ ID NO: 2700; SEQ ID NO: 2701; SEQ ID NO: 2702; SEQ ID NO: 2703; SEQ ID NO: 2704; SEQ ID NO: 2705; SEQ ID NO: 2706; SEQ ID NO: 2707; SEQ ID NO: 2708; SEQ ID NO: 2709; SEQ ID NO: 2710; SEQ ID NO: 2711; SEQ ID NO: 2712; SEQ ID NO: 2713; SEQ ID NO: 2714; SEQ ID NO: 2715; SEQ ID NO: 2716; SEQ ID NO: 2717; SEQ ID NO: 2718; SEQ ID NO: 2719; SEQ ID NO: 2720; SEQ ID NO: 2721; SEQ ID NO: 2722; SEQ ID NO: 2723; SEQ ID NO: 2724; SEQ ID NO: 2725; SEQ ID NO: 2726; SEQ ID NO: 2727; SEQ ID NO: 2728; SEQ ID NO: 2729; SEQ ID NO: 2730; SEQ ID NO: 2731; SEQ ID NO: 2732; SEQ ID NO: 2733; SEQ ID NO: 2734; SEQ ID NO: 2735; SEQ ID NO: 2736; SEQ ID NO: 2737; SEQ ID NO: 2738; SEQ ID NO: 2739; SEQ ID NO: 2740; SEQ ID NO: 2741; SEQ ID NO: 2742; SEQ ID NO: 2743; SEQ ID NO: 2744; SEQ ID NO: 2745; SEQ ID NO: 2746; SEQ ID NO: 2747; SEQ ID NO: 2748; SEQ ID NO: 2749; SEQ ID NO: 2750; SEQ ID NO: 2751; SEQ ID NO: 2752; SEQ ID NO: 2753; SEQ ID NO: 2754; SEQ ID NO: 2755; SEQ ID NO: 2756; SEQ ID NO: 2757; SEQ ID NO: 2758; SEQ ID NO: 2759; SEQ ID NO: 2760; SEQ ID NO: 2761; SEQ ID NO: 2762; SEQ ID NO: 2763; SEQ ID NO: 2764; SEQ ID NO: 2765; SEQ ID NO: 2766; SEQ ID NO: 2767; SEQ ID NO: 2768; SEQ ID NO: 2769; SEQ ID NO: 2770; SEQ ID NO: 2771; SEQ ID NO: 2772; SEQ ID NO: 2773; SEQ ID NO: 2774; SEQ ID NO: 2775; SEQ ID NO: 2776; SEQ ID NO: 2777; SEQ ID NO: 2778; SEQ ID NO: 2779; SEQ ID NO: 2780; SEQ ID NO: 2781; SEQ ID NO: 2782; SEQ ID NO: 2783; SEQ ID NO: 2784; SEQ ID NO: 2785; SEQ ID NO: 2786; SEQ ID NO: 2787; SEQ ID NO: 2788; SEQ ID NO: 2789; SEQ ID NO: 2790; SEQ ID NO: 2791; SEQ ID NO: 2792; SEQ ID NO: 2793; SEQ ID NO: 2794; SEQ ID NO: 2795; SEQ ID NO: 2796; SEQ ID NO: 2797; SEQ ID NO: 2798; SEQ ID NO: 2799; SEQ ID NO: 2800; SEQ ID NO: 2081; SEQ ID NO: 2802; SEQ ID NO: 2803; SEQ ID NO: 2804; SEQ ID NO: 2805; SEQ ID NO: 2806; SEQ ID NO: 2807; SEQ ID NO: 2808; SEQ ID NO: 2809; SEQ ID NO: 2810; SEQ ID NO: 2811; SEQ ID NO: 2812; SEQ ID NO: 2813; SEQ ID NO: 2814; SEQ ID NO: 2815; SEQ ID NO: 2816; SEQ ID NO: 2817; SEQ ID NO: 2818; SEQ ID NO: 2819; SEQ ID NO: 2820; SEQ ID NO: 2821; SEQ ID NO: 2822; SEQ ID NO: 2823; SEQ ID NO: 2824; SEQ ID NO: 2825; SEQ ID NO: 2826; SEQ ID NO: 2827; SEQ ID NO: 2828; SEQ ID NO: 2829; SEQ ID NO: 2830; SEQ ID NO: 2831; SEQ ID NO: 2832; SEQ ID NO: 2833; SEQ ID NO: 2834; SEQ ID NO: 2835; SEQ ID NO: 2836; SEQ ID NO: 2837; SEQ ID NO: 2838; SEQ ID NO: 2839; SEQ ID NO: 2840; SEQ ID NO: 2841; SEQ ID NO: 2842; SEQ ID NO: 2843; SEQ ID NO: 2844; SEQ ID NO: 2845; SEQ ID NO: 2846; SEQ ID NO: 2847; SEQ ID NO: 2848; SEQ ID NO: 2849; SEQ ID NO: 2850; SEQ ID NO: 2851; SEQ ID NO: 2852; SEQ ID NO: 2853; SEQ ID NO: 2854; SEQ ID NO: 2855; SEQ ID NO: 2856; SEQ ID NO: 2857; SEQ ID NO: 2858; SEQ ID NO: 2859; SEQ ID NO: 2860; SEQ ID NO: 2861; SEQ ID NO: 2862; SEQ ID NO: 2863; SEQ ID NO: 2864; SEQ ID NO: 2865; SEQ ID NO: 2866; SEQ ID NO: 2867; SEQ ID NO: 2868; SEQ ID NO: 2869; SEQ ID NO: 2870; SEQ ID NO: 2871; SEQ ID NO: 2872; SEQ ID NO: 2873; SEQ ID NO: 2874; SEQ ID NO: 2875; SEQ ID NO: 2876; SEQ ID NO: 2877; SEQ ID NO: 2878; SEQ ID NO: 2879; SEQ ID NO: 2880; SEQ ID NO: 2881; SEQ ID NO: 2882; SEQ ID NO: 2883; SEQ ID NO: 2884; SEQ ID NO: 2885; SEQ ID NO: 2886; SEQ ID NO: 2887; SEQ ID NO: 2888; SEQ ID NO: 2889; SEQ ID NO: 2890; SEQ ID NO: 2891; SEQ ID NO: 2892; SEQ ID NO: 2893; SEQ ID NO: 2894; SEQ ID NO: 2895; SEQ ID NO: 2896; SEQ ID NO: 2897; SEQ ID NO: 2898; SEQ ID NO: 2899; SEQ ID NO: 2900; SEQ ID NO: 2901; SEQ ID NO: 2902; SEQ ID NO: 2903; SEQ ID NO: 2904; SEQ ID NO: 2905; SEQ ID NO: 2906; SEQ ID NO: 2907; SEQ ID NO: 2908; SEQ ID NO: 2909; SEQ ID NO: 2910; SEQ ID NO: 2911; SEQ ID NO: 2912; SEQ ID NO: 2913; SEQ ID NO: 2914; SEQ ID NO: 2915; SEQ ID NO: 2916; SEQ ID NO: 2917; SEQ ID NO: 2918; SEQ ID NO: 2919; SEQ ID NO: 2920; SEQ ID NO: 2921; SEQ ID NO: 2922; SEQ ID NO: 2923; SEQ ID NO: 2924; SEQ ID NO: 2925; SEQ ID NO: 2926; SEQ ID NO: 2927; SEQ ID NO: 2928; SEQ ID NO: 2929; SEQ ID NO: 2930; SEQ ID NO: 2931; SEQ ID NO: 2932; SEQ ID NO: 2933; SEQ ID NO: 2934; SEQ ID NO: 2935; SEQ ID NO: 2936; SEQ ID NO: 2937; SEQ ID NO: 2938; SEQ ID NO: 2939; SEQ ID NO: 2940; SEQ ID NO: 2941; SEQ ID NO: 2942; SEQ ID NO: 2943; SEQ ID NO: 2944; SEQ ID NO: 2945; SEQ ID NO: 2946; SEQ ID NO: 2947; SEQ ID NO: 2948; SEQ ID NO: 2949; SEQ ID NO: 2950; SEQ ID NO: 2951; SEQ ID NO: 2952; SEQ ID NO: 2953; SEQ ID NO: 2954; SEQ ID NO: 2955; SEQ ID NO: 2956; SEQ ID NO: 2957; SEQ ID NO: 2958; SEQ ID NO: 2959; SEQ ID NO: 2960; SEQ ID NO: 2961; SEQ ID NO: 2962; SEQ ID NO: 2963; SEQ ID NO: 2964; SEQ ID NO: 2965; SEQ ID NO: 2966; SEQ ID NO: 2967; SEQ ID NO: 2968; SEQ ID NO: 2969; SEQ ID NO: 2970; SEQ ID NO: 2971; SEQ ID NO: 2972; SEQ ID NO: 2973; SEQ ID NO: 2974; SEQ ID NO: 2975; SEQ ID NO: 2976; SEQ ID NO: 2977; SEQ ID NO: 2978; SEQ ID NO: 2979; SEQ ID NO: 2980; SEQ ID NO: 2981; SEQ ID NO: 2982; SEQ ID NO: 2983; SEQ ID NO: 2984; SEQ ID NO: 2985; SEQ ID NO: 2986; SEQ ID NO: 2987; SEQ ID NO: 2988; SEQ ID NO: 2989; SEQ ID NO: 2990; SEQ ID NO: 2991; SEQ ID NO: 2992; SEQ ID NO: 2993; SEQ ID NO: 2994; SEQ ID NO: 2995; SEQ ID NO: 2996; SEQ ID NO: 2997; SEQ ID NO: 2998; SEQ ID NO: 2999; SEQ ID NO: 3000; SEQ ID NO: 3001; SEQ ID NO: 3002; SEQ ID NO: 3003; SEQ ID NO: 3004; SEQ ID NO: 3005; SEQ ID NO: 3006; SEQ ID NO: 3007; SEQ ID NO: 3008; SEQ ID NO: 3009; SEQ ID NO: 3010; SEQ ID NO: 3011; SEQ ID NO: 3012; SEQ ID NO: 3013; SEQ ID NO: 3014; SEQ ID NO: 3015; SEQ ID NO: 3016; SEQ ID NO: 3017; SEQ ID NO: 3018; SEQ ID NO: 3019; SEQ ID NO: 3020; SEQ ID NO: 3021; SEQ ID NO: 3022; SEQ ID NO: 3023; SEQ ID NO: 3024; SEQ ID NO: 3025; SEQ ID NO: 3026; SEQ ID NO: 3027; SEQ ID NO: 3028; SEQ ID NO: 3029; SEQ ID NO: 3030; SEQ ID NO: 3031; SEQ ID NO: 3032; SEQ ID NO: 3033; SEQ ID NO: 3034; SEQ ID NO: 3035; SEQ ID NO: 3036; SEQ ID NO: 3037; SEQ ID NO: 3038; SEQ ID NO: 3039; SEQ ID NO: 3040; SEQ ID NO: 3041; SEQ ID NO: 3042; SEQ ID NO: 3043; SEQ ID NO: 3044; SEQ ID NO: 3045; SEQ ID NO: 3046; SEQ ID NO: 3047; SEQ ID NO: 3048; SEQ ID NO: 3049; SEQ ID NO: 3050; SEQ ID NO: 3051; SEQ ID NO: 3052; SEQ ID NO: 3053; SEQ ID NO: 3054; SEQ ID NO: 3055; SEQ ID NO: 3056; SEQ ID NO: 3057; SEQ ID NO: 3058; SEQ ID NO: 3059; SEQ ID NO: 3060; SEQ ID NO: 3061; SEQ ID NO: 3062; SEQ ID NO: 3063; SEQ ID NO: 3064; SEQ ID NO: 3065; SEQ ID NO: 3066; SEQ ID NO: 3067; SEQ ID NO: 3068; SEQ ID NO: 3069; SEQ ID NO: 3070; SEQ ID NO: 3071; SEQ ID NO: 3072; SEQ ID NO: 3073; SEQ ID NO: 3074; SEQ ID NO: 3075; SEQ ID NO: 3076; SEQ ID NO: 3077; SEQ ID NO: 3078; SEQ ID NO: 3079; SEQ ID NO: 3080; SEQ ID NO: 3081; SEQ ID NO: 3082; SEQ ID NO: 3083; SEQ ID NO: 3084; SEQ ID NO: 3085; SEQ ID NO: 3086; SEQ ID NO: 3087; SEQ ID NO: 3088; SEQ ID NO: 3089; SEQ ID NO: 3090; SEQ ID NO: 3091; SEQ ID NO: 3092; SEQ ID NO: 3093; SEQ ID NO: 3094; SEQ ID NO: 3095; SEQ ID NO: 3096; SEQ ID NO: 3097; SEQ ID NO: 3098; SEQ ID NO: 3099; SEQ ID NO: 3100; SEQ ID NO: 3101; SEQ ID NO: 3102; SEQ ID NO: 3103; SEQ ID NO: 3104; SEQ ID NO: 3105; SEQ ID NO: 3106; SEQ ID NO: 3107; SEQ ID NO: 3108; SEQ ID NO: 3109; SEQ ID NO: 3110; SEQ ID NO: 3111; SEQ ID NO: 3112; SEQ ID NO: 3113; SEQ ID NO: 3114; SEQ ID NO: 3115; SEQ ID NO: 3116; SEQ ID NO: 3117; SEQ ID NO: 3118; SEQ ID NO: 3119; SEQ ID NO: 3120; SEQ ID NO: 3121; SEQ ID NO: 3122; SEQ ID NO: 3123; SEQ ID NO: 3124; SEQ ID NO: 3125; SEQ ID NO: 3126; SEQ ID NO: 3127; SEQ ID NO: 3128; SEQ ID NO: 3129; SEQ ID NO: 3130; SEQ ID NO: 3131; SEQ ID NO: 3132; SEQ ID NO: 3133; SEQ ID NO: 3134; SEQ ID NO: 3135; SEQ ID NO: 3136; SEQ ID NO: 3137; SEQ ID NO:3138; SEQ ID NO: 3139; SEQ ID NO: 3140; SEQ ID NO: 3141; SEQ ID NO: 3142; SEQ ID NO: 3143; SEQ ID NO: 3144; SEQ ID NO: 3145; SEQ ID NO: 3146; SEQ ID NO: 3147; SEQ ID NO: 3148; SEQ ID NO: 3149; SEQ ID NO: 3150; SEQ ID NO: 3151; SEQ ID NO: 3152; SEQ ID NO: 3153; SEQ ID NO: 3154; SEQ ID NO: 3155; SEQ ID NO: 3156; SEQ ID NO: 3157; SEQ ID NO: 3158; SEQ ID NO: 3159; SEQ ID NO: 3160; SEQ ID NO: 3161; SEQ ID NO: 3162; SEQ ID NO: 3163; SEQ ID NO: 3164; SEQ ID NO: 3165; SEQ ID NO: 3166; SEQ ID NO: 3167; SEQ ID NO: 3168; SEQ ID NO: 3169; SEQ ID NO: 3170; SEQ ID NO: 3171; SEQ ID NO: 3172; SEQ ID NO: 3173; SEQ ID NO: 3174; SEQ ID NO: 3175; SEQ ID NO: 3176; SEQ ID NO: 3177; SEQ ID NO: 3178; SEQ ID NO: 3179; SEQ ID NO: 3180; SEQ ID NO: 3181; SEQ ID NO: 3182; SEQ ID NO: 3183; SEQ ID NO: 3184; SEQ ID NO: 3185; SEQ ID NO: 3186; SEQ ID NO: 3187; SEQ ID NO: 3188; SEQ ID NO: 3189; SEQ ID NO: 3190; SEQ ID NO: 3191; SEQ ID NO: 3192; SEQ ID NO: 3193; SEQ ID NO: 3194; SEQ ID NO: 3195; SEQ ID NO: 3196; SEQ ID NO: 3197; SEQ ID NO: 3198; SEQ ID NO: 3199; SEQ ID NO: 3200; SEQ ID NO: 3201; SEQ ID NO: 3202; SEQ ID NO: 3203; SEQ ID NO: 3204; SEQ ID NO: 3205; SEQ ID NO: 3206; SEQ ID NO: 3207; SEQ ID NO: 3208; SEQ ID NO: 3209; SEQ ID NO: 3210; SEQ ID NO: 3211; SEQ ID NO: 3212; SEQ ID NO: 3213; SEQ ID NO: 3214; SEQ ID NO: 3215; SEQ ID NO: 3216; SEQ ID NO: 3217; SEQ ID NO: 3218; SEQ ID NO: 3219; SEQ ID NO: 3220; SEQ ID NO: 3221; SEQ ID NO: 3222; SEQ ID NO: 3223; SEQ ID NO: 3224; SEQ ID NO: 3225; SEQ ID NO: 3226; SEQ ID NO: 3227; SEQ ID NO: 3228; SEQ ID NO: 3229; SEQ ID NO: 3230; SEQ ID NO: 3231; SEQ ID NO: 3232; SEQ ID NO: 3233; SEQ ID NO: 3234; SEQ ID NO: 3235; SEQ ID NO: 3236; SEQ ID NO: 3237; SEQ ID NO: 3238; SEQ ID NO: 3239; SEQ ID NO: 3240; SEQ ID NO: 3241; SEQ ID NO: 3242; SEQ ID NO: 3243; SEQ ID NO: 3244; SEQ ID NO: 3245; SEQ ID NO: 3246; SEQ ID NO: 3247; SEQ ID NO: 3248; SEQ ID NO: 3249; SEQ ID NO: 3250; SEQ ID NO: 3251; SEQ ID NO: 3252; SEQ ID NO: 3253; SEQ ID NO: 3254; SEQ ID NO: 3255; SEQ ID NO: 3256; SEQ ID NO: 3257; SEQ ID NO: 3258; SEQ ID NO: 3259; SEQ ID NO: 3260; SEQ ID NO: 3261; SEQ ID NO: 3262; SEQ ID NO: 3263; SEQ ID NO: 3264; SEQ ID NO: 3265; SEQ ID NO: 3266; SEQ ID NO: 3267; SEQ ID NO: 3268; SEQ ID NO: 3269; SEQ ID NO: 3270; SEQ ID NO: 3271; SEQ ID NO: 3272; SEQ ID NO: 3273; SEQ ID NO: 3274; SEQ ID NO: 3275; SEQ ID NO: 3276; SEQ ID NO: 3277; SEQ ID NO: 3278; SEQ ID NO: 3279; SEQ ID NO: 3280; SEQ ID NO: 3281; SEQ ID NO: 3282; SEQ ID NO: 3283; SEQ ID NO: 3284; SEQ ID NO: 3285; SEQ ID NO: 3286; SEQ ID NO: 3287; SEQ ID NO: 3288; SEQ ID NO: 3289; SEQ ID NO: 3290; SEQ ID NO: 3291; SEQ ID NO: 3292; SEQ ID NO: 3293; SEQ ID NO: 3294; SEQ ID NO: 3295; SEQ ID NO: 3296; SEQ ID NO: 3297; SEQ ID NO: 3298; SEQ ID NO: 3299; SEQ ID NO: 3300; SEQ ID NO: 3301; SEQ ID NO: 3302; SEQ ID NO: 3303; SEQ ID NO: 3304; SEQ ID NO: 3305; SEQ ID NO: 3306; SEQ ID NO: 3307; SEQ ID NO: 3308; SEQ ID NO: 3309; SEQ ID NO: 3310; SEQ ID NO: 3311; SEQ ID NO: 3312; SEQ ID NO: 3313; SEQ ID NO: 3314; SEQ ID NO: 3315; SEQ ID NO: 3316; SEQ ID NO: 3317; SEQ ID NO: 3318; SEQ ID NO: 3319; SEQ ID NO: 3320; SEQ ID NO: 3321; SEQ ID NO: 3322; SEQ ID NO: 3323; SEQ ID NO: 3324; SEQ ID NO: 3325; SEQ ID NO: 3326; SEQ ID NO: 3327; SEQ ID NO: 3328; SEQ ID NO: 3329; SEQ ID NO: 3330; SEQ ID NO: 3331; SEQ ID NO: 3332; SEQ ID NO: 3333; SEQ ID NO: 3334; SEQ ID NO: 3335; SEQ ID NO: 3336; SEQ ID NO: 3337; SEQ ID NO: 3338; SEQ ID NO: 3339; SEQ ID NO: 3340; SEQ ID NO: 3341; SEQ ID NO: 3342; SEQ ID NO: 3343; SEQ ID NO: 3344; SEQ ID NO: 3345; SEQ ID NO: 3346; SEQ ID NO: 3347; SEQ ID NO: 3348; SEQ ID NO: 3349; SEQ ID NO: 3350; SEQ ID NO: 3351; SEQ ID NO: 3352; SEQ ID NO: 3353; SEQ ID NO: 3354; SEQ ID NO: 3355; SEQ ID NO: 3356; SEQ ID NO: 3357; SEQ ID NO: 3358; SEQ ID NO: 3359; SEQ ID NO: 3360; SEQ ID NO: 3361; SEQ ID NO: 3362; SEQ ID NO: 3363; SEQ ID NO: 3364; SEQ ID NO: 3365; SEQ ID NO: 3366; SEQ ID NO: 3367; SEQ ID NO: 3368; SEQ ID NO: 3369; SEQ ID NO: 3370; SEQ ID NO: 3371; SEQ ID NO: 3372; SEQ ID NO: 3373; SEQ ID NO: 3374; SEQ ID NO: 3375; SEQ ID NO: 3376; SEQ ID NO: 3377; SEQ ID NO: 3378; SEQ ID NO: 3379; SEQ ID NO: 3380; SEQ ID NO: 3381; SEQ ID NO: 3382; SEQ ID NO: 3383; SEQ ID NO: 3384; SEQ ID NO: 3385; SEQ ID NO: 3386; SEQ ID NO: 3387; SEQ ID NO: 3388; SEQ ID NO: 3389; SEQ ID NO: 3390; SEQ ID NO: 3391; SEQ ID NO: 3392; SEQ ID NO: 3393; SEQ ID NO: 3394; SEQ ID NO: 3395; SEQ ID NO: 3396; SEQ ID NO: 3397; SEQ ID NO: 3398; SEQ ID NO: 3399; SEQ ID NO: 3400; SEQ ID NO: 3401; SEQ ID NO: 3402; SEQ ID NO: 3403; SEQ ID NO: 3404; SEQ ID NO: 3405; SEQ ID NO: 3406; SEQ ID NO: 3407; SEQ ID NO: 3408; SEQ ID NO: 3409; SEQ ID NO: 3410; SEQ ID NO: 3411; SEQ ID NO: 3412; SEQ ID NO: 3413; SEQ ID NO: 3414; SEQ ID NO: 3415; SEQ ID NO: 3416; SEQ ID NO: 3417; SEQ ID NO: 3418; SEQ ID NO: 3419; SEQ ID NO: 3420; SEQ ID NO: 3421; SEQ ID NO: 3422; SEQ ID NO: 3423; SEQ ID NO: 3424; SEQ ID NO: 3425; SEQ ID NO: 3426; SEQ ID NO: 3427; SEQ ID NO: 3428; SEQ ID NO: 3429; SEQ ID NO: 3430; SEQ ID NO: 3431; SEQ ID NO: 3432; SEQ ID NO: 3433; SEQ ID NO: 3434; SEQ ID NO: 3435; SEQ ID NO: 3436; SEQ ID NO: 3437; SEQ ID NO: 3438; SEQ ID NO: 3439; SEQ ID NO: 3440; SEQ ID NO: 3441; SEQ ID NO: 3442; SEQ ID NO: 3443; SEQ ID NO: 3444; SEQ ID NO: 3445; SEQ ID NO: 3446; SEQ ID NO: 3447; SEQ ID NO: 3448; SEQ ID NO: 3449; SEQ ID NO: 3450; SEQ ID NO: 3451; SEQ ID NO: 3452; SEQ ID NO: 3453; SEQ ID NO: 3454; SEQ ID NO: 3455; SEQ ID NO: 3456; SEQ ID NO: 3457; SEQ ID NO: 3458; SEQ ID NO: 3459, SEQ ID NO: 3460; SEQ ID NO: 3461; SEQ ID NO: 3462; SEQ ID NO: 3463; SEQ ID NO: 3464; SEQ ID NO: 3465; SEQ ID NO: 3466; SEQ ID NO: 3467; SEQ ID NO: 3468; SEQ ID NO: 3469; SEQ ID NO: 3470; SEQ ID NO: 3471; SEQ ID NO: 3472; SEQ ID NO: 3473; SEQ ID NO: 3474; SEQ ID NO: 3475; SEQ ID NO: 3476; SEQ ID NO: 3477; SEQ ID NO: 3478; SEQ ID NO: 3479; SEQ ID NO: 3480; SEQ ID NO: 3481; SEQ ID NO: 3482; SEQ ID NO: 3483; SEQ ID NO: 3484; SEQ ID NO: 3485; SEQ ID NO: 3486; SEQ ID NO: 3487; SEQ ID NO: 3488; SEQ ID NO: 3489; SEQ ID NO: 3490; SEQ ID NO: 3491; SEQ ID NO: 3492; SEQ ID NO: 3493; SEQ ID NO: 3494; SEQ ID NO: 3495; SEQ ID NO: 3496; SEQ ID NO: 3497; SEQ ID NO: 3498; SEQ ID NO: 3499; SEQ ID NO: 3500; SEQ ID NO: 3501; SEQ ID NO: 3502; SEQ ID NO: 3503; SEQ ID NO: 3504; SEQ ID NO: 3505; SEQ ID NO: 3506; SEQ ID NO: 3507; SEQ ID NO: 3508; SEQ ID NO: 3509; SEQ ID NO: 3510; SEQ ID NO: 3511; SEQ ID NO: 3512; SEQ ID NO: 3513; SEQ ID NO: 3514; SEQ ID NO: 3515; SEQ ID NO: 3516; SEQ ID NO: 3517; SEQ ID NO: 3518; SEQ ID NO: 3519; SEQ ID NO: 3520; SEQ ID NO: 3521; SEQ ID NO: 3522; SEQ ID NO: 3523; SEQ ID NO: 3524; SEQ ID NO: 3525; SEQ ID NO: 3526; SEQ ID NO: 3527; SEQ ID NO: 3528; SEQ ID NO: 3529; SEQ ID NO: 3530; SEQ ID NO: 3531; SEQ ID NO: 3532; SEQ ID NO: 3533; SEQ ID NO: 3534; SEQ ID NO: 3535; SEQ ID NO: 3536; SEQ ID NO: 3537; SEQ ID NO: 3538; SEQ ID NO: 3539; SEQ ID NO: 3540; SEQ ID NO: 3541; SEQ ID NO: 3542; SEQ ID NO: 3543; SEQ ID NO: 3544; SEQ ID NO: 3545; SEQ ID NO: 3546; SEQ ID NO: 3547; SEQ ID NO: 3548; SEQ ID NO: 3549; SEQ ID NO: 3550; SEQ ID NO: 3551; SEQ ID NO: 3552; SEQ ID NO: 3553; SEQ ID NO: 3554; SEQ ID NO: 3555; SEQ ID NO: 3556; SEQ ID NO: 3557; SEQ ID NO: 3558; SEQ ID NO: 3559; SEQ ID NO: 3560; SEQ ID NO: 3561; SEQ ID NO: 3562; SEQ ID NO: 3563; SEQ ID NO: 3564; SEQ ID NO: 3565; SEQ ID NO: 3566; SEQ ID NO: 3567; SEQ ID NO: 3568; SEQ ID NO: 3569; SEQ ID NO: 3570; SEQ ID NO: 3571; SEQ ID NO: 3572; SEQ ID NO: 3573; SEQ ID NO: 3574; SEQ ID NO: 3575; SEQ ID NO: 3576; SEQ ID NO: 3577; SEQ ID NO: 3578; SEQ ID NO: 3579; SEQ ID NO: 3580; SEQ ID NO: 3581; SEQ ID NO: 3582; SEQ ID NO: 3583; SEQ ID NO: 3584; SEQ ID NO: 3585; SEQ ID NO: 3586; SEQ ID NO: 3587; SEQ ID NO: 3588; SEQ ID NO: 3589; SEQ ID NO: 3590; SEQ ID NO: 3591; SEQ ID NO: 3592; SEQ ID NO: 3593; SEQ ID NO: 3594; SEQ ID NO: 3595; SEQ ID NO: 3596; SEQ ID NO: 3597; SEQ ID NO: 3598; SEQ ID NO: 3599; SEQ ID NO: 3600; SEQ ID NO: 3601; SEQ ID NO: 3602; SEQ ID NO: 3603; SEQ ID NO: 3604; SEQ ID NO: 3605; SEQ ID NO: 3606; SEQ ID NO: 3607; SEQ ID NO: 3608; SEQ ID NO: 3609; SEQ ID NO: 3610; SEQ ID NO: 3611; SEQ ID NO: 3612; SEQ ID NO: 3613; SEQ ID NO: 3614; SEQ ID NO: 3615; SEQ ID NO: 3616; SEQ ID NO: 3617; SEQ ID NO: 3618; SEQ ID NO: 3619; SEQ ID NO: 3620; SEQ ID NO: 3621; SEQ ID NO: 3622; SEQ ID NO: 3623; SEQ ID NO: 3624; SEQ ID NO: 3625; SEQ ID NO: 3626; SEQ ID NO: 3627; SEQ ID NO: 3628; SEQ ID NO: 3629; SEQ ID NO: 3630; SEQ ID NO: 3631; SEQ ID NO: 3632; SEQ ID NO: 3633; SEQ ID NO: 3634; SEQ ID NO: 3635; SEQ ID NO: 3636; SEQ ID NO: 3637; SEQ ID NO: 3638; SEQ ID NO: 3639; SEQ ID NO: 3640; SEQ ID NO: 3641; SEQ ID NO: 3642; SEQ ID NO: 3643; SEQ ID NO: 3644; SEQ ID NO: 3645; SEQ ID NO: 3646; SEQ ID NO: 3647; SEQ ID NO: 3648; SEQ ID NO: 3649; SEQ ID NO: 3650; SEQ ID NO: 3651; SEQ ID NO: 3652; SEQ ID NO: 3653; SEQ ID NO: 3654; SEQ ID NO: 3655; SEQ ID NO: 3656; SEQ ID NO: 3657; SEQ ID NO: 3658; SEQ ID NO: 3659; SEQ ID NO: 3660; SEQ ID NO: 3661; SEQ ID NO: 3662; SEQ ID NO: 3663; SEQ ID NO: 3664; SEQ ID NO: 3665; SEQ ID NO: 3666; SEQ ID NO: 3667; SEQ ID NO: 3668; SEQ ID NO: 3669; SEQ ID NO: 3670; SEQ ID NO: 3671; SEQ ID NO: 3672; SEQ ID NO: 3673; SEQ ID NO: 3674; SEQ ID NO: 3675; SEQ ID NO: 3676; SEQ ID NO: 3677; SEQ ID NO: 3678; SEQ ID NO: 3679; SEQ ID NO: 3680; SEQ ID NO: 3681; SEQ ID NO: 3682; SEQ ID NO: 3683; SEQ ID NO: 3684; SEQ ID NO: 3685; SEQ ID NO: 3686; SEQ ID NO: 3687; SEQ ID NO: 3688; SEQ ID NO: 3689; SEQ ID NO: 3690; SEQ ID NO: 3691; SEQ ID NO: 3692; SEQ ID NO: 3693; SEQ ID NO: 3694; SEQ ID NO: 3695; SEQ ID NO: 3696; SEQ ID NO: 3697; SEQ ID NO: 3698; SEQ ID NO: 3699; SEQ ID NO: 3700; SEQ ID NO: 3701; SEQ ID NO: 3702; SEQ ID NO: 3703; SEQ ID NO: 3704; SEQ ID NO: 3705; SEQ ID NO: 3706; SEQ ID NO: 3707; SEQ ID NO: 3708; SEQ ID NO: 3709; SEQ ID NO: 3710; SEQ ID NO: 3711; SEQ ID NO: 3712; SEQ ID NO: 3713; SEQ ID NO: 3714; SEQ ID NO: 3715; SEQ ID NO: 3716; SEQ ID NO: 3717; SEQ ID NO: 3718; SEQ ID NO: 3719; SEQ ID NO: 3720; SEQ ID NO: 3721; SEQ ID NO: 3722; SEQ ID NO: 3723; SEQ ID NO: 3724; SEQ ID NO: 3725; SEQ ID NO: 3726; SEQ ID NO: 3727; SEQ ID NO: 3728; SEQ ID NO: 3729; SEQ ID NO: 3730; SEQ ID NO: 3731; SEQ ID NO: 3732; SEQ ID NO: 3733; SEQ ID NO: 3734; SEQ ID NO: 3735; SEQ ID NO: 3736; SEQ ID NO: 3737; SEQ ID NO: 3738; SEQ ID NO: 3739; SEQ ID NO: 3740; SEQ ID NO: 3741; SEQ ID NO: 3742; SEQ ID NO: 3743; SEQ ID NO: 3744; SEQ ID NO: 3745; SEQ ID NO: 3746; SEQ ID NO: 3747; SEQ ID NO: 3748; SEQ ID NO: 3749; SEQ ID NO: 3750; SEQ ID NO: 3751; SEQ ID NO: 3752; SEQ ID NO: 3753; SEQ ID NO: 3754; SEQ ID NO: 3755; SEQ ID NO: 3756; SEQ ID NO: 3757; SEQ ID NO: 3758; SEQ ID NO: 3759; SEQ ID NO: 3760; SEQ ID NO: 3761; SEQ ID NO: 3762; SEQ ID NO: 3763; SEQ ID NO: 3764; SEQ ID NO: 3765; SEQ ID NO: 3766; SEQ ID NO: 3767; SEQ ID NO: 3768; SEQ ID NO: 3769; SEQ ID NO: 3770; SEQ ID NO: 3771; SEQ ID NO: 3772; SEQ ID NO: 3773; SEQ ID NO: 3774; SEQ ID NO: 3775; SEQ ID NO: 3776; SEQ ID NO: 3777; SEQ ID NO: 3778; SEQ ID NO: 3779; SEQ ID NO: 3780; SEQ ID NO: 3781; SEQ ID NO: 3782; SEQ ID NO: 3783; SEQ ID NO: 3784; SEQ ID NO: 3785; SEQ ID NO: 3786; SEQ ID NO: 3787; SEQ ID NO: 3788; SEQ ID NO: 3789; SEQ ID NO: 3790; SEQ ID NO: 3791; SEQ ID NO: 3792; SEQ ID NO: 3793; SEQ ID NO: 3794; SEQ ID NO: 3795; SEQ ID NO: 3796; SEQ ID NO: 3797; SEQ ID NO: 3798; SEQ ID NO: 3799; SEQ ID NO: 3800; SEQ ID NO: 3801; SEQ ID NO: 3802; SEQ ID NO: 3803; SEQ ID NO: 3804; SEQ ID NO: 3805; SEQ ID NO: 3806; SEQ ID NO: 3807; SEQ ID NO: 3808; SEQ ID NO: 3809; SEQ ID NO: 3810; SEQ ID NO: 3811; SEQ ID NO: 3812; SEQ ID NO: 3813; SEQ ID NO: 3814; SEQ ID NO: 3815; SEQ ID NO: 3816; SEQ ID NO: 3817; SEQ ID NO: 3818; SEQ ID NO: 3819; SEQ ID NO: 3820; SEQ ID NO: 3821; SEQ ID NO: 3822; SEQ ID NO: 3823; SEQ ID NO: 3824; SEQ ID NO: 3825; SEQ ID NO: 3826; SEQ ID NO: 3827; SEQ ID NO: 3828; SEQ ID NO: 3829; SEQ ID NO: 3830; SEQ ID NO: 3831; SEQ ID NO: 3832; SEQ ID NO: 3833; SEQ ID NO: 3834; SEQ ID NO: 3835; SEQ ID NO: 3836; SEQ ID NO: 3837; SEQ ID NO: 3838; SEQ ID NO: 3839; SEQ ID NO: 3840; SEQ ID NO: 3841; SEQ ID NO: 3842; SEQ ID NO: 3843; SEQ ID NO: 3844; SEQ ID NO: 3845; SEQ ID NO: 3846; SEQ ID NO: 3847; SEQ ID NO: 3848; SEQ ID NO: 3849; SEQ ID NO: 3850; SEQ ID NO: 3851; SEQ ID NO: 3852; SEQ ID NO: 3853; SEQ ID NO: 3854; SEQ ID NO: 3855; SEQ ID NO: 3856; SEQ ID NO: 3857; SEQ ID NO: 3858; SEQ ID NO: 3859; SEQ ID NO: 3860; SEQ ID NO: 3861; SEQ ID NO: 3862; SEQ ID NO: 3863; SEQ ID NO: 3864; SEQ ID NO: 3865; SEQ ID NO: 3866; SEQ ID NO: 3867; SEQ ID NO: 3868; SEQ ID NO: 3869; SEQ ID NO: 3870; SEQ ID NO: 3871; SEQ ID NO: 3872; SEQ ID NO: 3873; SEQ ID NO: 3874; SEQ ID NO: 3875; SEQ ID NO: 3876; SEQ ID NO: 3877; SEQ ID NO: 3878; SEQ ID NO: 3879; SEQ ID NO: 3880; SEQ ID NO: 3881; SEQ ID NO: 3882; SEQ ID NO: 3883; SEQ ID NO: 3884; SEQ ID NO: 3885; SEQ ID NO: 3886; SEQ ID NO: 3887; SEQ ID NO: 3888; SEQ ID NO: 3889; SEQ ID NO: 3890; SEQ ID NO: 3891; SEQ ID NO: 3892; SEQ ID NO: 3893; SEQ ID NO: 3894; SEQ ID NO: 3895; SEQ ID NO: 3896; SEQ ID NO: 3897; SEQ ID NO: 3898; SEQ ID NO: 3899; SEQ ID NO: 3900; SEQ ID NO: 3901; SEQ ID NO: 3902; SEQ ID NO: 3903; SEQ ID NO: 3904; SEQ ID NO: 3905; SEQ ID NO: 3906; SEQ ID NO: 3907; SEQ ID NO: 3908; SEQ ID NO: 3909; SEQ ID NO: 3910; SEQ ID NO: 3911; SEQ ID NO: 3912; SEQ ID NO: 3913; SEQ ID NO: 3914; SEQ ID NO: 3915; SEQ ID NO: 3916; SEQ ID NO: 3917; SEQ ID NO: 3918; SEQ ID NO: 3919; SEQ ID NO: 3920; SEQ ID NO: 3921; SEQ ID NO: 3922; SEQ ID NO: 3923; SEQ ID NO: 3924; SEQ ID NO: 3925; SEQ ID NO: 3926; SEQ ID NO: 3927; SEQ ID NO: 3928; SEQ ID NO: 3929; SEQ ID NO: 3930; SEQ ID NO: 3931; SEQ ID NO: 3932; SEQ ID NO: 3933; SEQ ID NO: 3934; SEQ ID NO: 3935; SEQ ID NO: 3936; SEQ ID NO: 3937; SEQ ID NO: 3938; SEQ ID NO: 3939; SEQ ID NO: 3940; SEQ ID NO: 3941; SEQ ID NO: 3942; SEQ ID NO: 3943; SEQ ID NO: 3944; SEQ ID NO: 3945; SEQ ID NO: 3946; SEQ ID NO: 3947; SEQ ID NO: 3948; SEQ ID NO: 3949; SEQ ID NO: 3950; SEQ ID NO: 3951; SEQ ID NO: 3952; SEQ ID NO: 3953; SEQ ID NO: 3954; SEQ ID NO: 3955; SEQ ID NO: 3956; SEQ ID NO: 3957; SEQ ID NO: 3958; SEQ ID NO: 3959; SEQ ID NO: 3960; SEQ ID NO: 3961; SEQ ID NO: 3962; SEQ ID NO: 3963; SEQ ID NO: 3964; SEQ ID NO: 3965; SEQ ID NO: 3966; SEQ ID NO: 3967; SEQ ID NO: 3968; SEQ ID NO: 3969; SEQ ID NO: 3970; SEQ ID NO: 3971; SEQ ID NO: 3972; SEQ ID NO: 3973; SEQ ID NO: 3974; SEQ ID NO: 3975; SEQ ID NO: 3976; SEQ ID NO: 3977; SEQ ID NO: 3978; SEQ ID NO: 3979; SEQ ID NO: 3980; SEQ ID NO: 3981; SEQ ID NO: 3982; SEQ ID NO: 3983; SEQ ID NO: 3984; SEQ ID NO: 3985; SEQ ID NO: 3986; SEQ ID NO: 3987; SEQ ID NO: 3988; SEQ ID NO: 3989; SEQ ID NO: 3990; SEQ ID NO: 3991; SEQ ID NO: 3992; SEQ ID NO: 3993; SEQ ID NO: 3994; SEQ ID NO: 3995; SEQ ID NO: 3996; SEQ ID NO: 3997; SEQ ID NO: 3998; SEQ ID NO: 3999; SEQ ID NO: 4000; SEQ ID NO: 4001; SEQ ID NO: 4002; SEQ ID NO: 4003; SEQ ID NO: 4004; SEQ ID NO: 4005; SEQ ID NO: 4006; SEQ ID NO: 4007; SEQ ID NO: 4008; SEQ ID NO: 4009; SEQ ID NO: 4010; SEQ ID NO: 4011; SEQ ID NO: 4012; SEQ ID NO: 4013; SEQ ID NO: 4014; SEQ ID NO: 4015; and SEQ ID NO: 9593; SEQ ID NO: 9594; SEQ ID NO: 9595; SEQ ID NO: 9596; SEQ ID NO: 9597; SEQ ID NO: 9598; SEQ ID NO: 9599; SEQ ID NO: 9600; SEQ ID NO: 9601; SEQ ID NO: 9602; SEQ ID NO: 9603; SEQ ID NO: 9604; SEQ ID NO: 9605; SEQ ID NO: 9606; SEQ ID NO: 9607; SEQ ID NO: 9608; SEQ ID NO: 9609; SEQ ID NO: 9610; SEQ ID NO: 9611; SEQ ID NO: 9612; SEQ ID NO: 9613; SEQ ID NO: 9614; SEQ ID NO: 9615; SEQ ID NO: 9616; SEQ ID NO: 9617; SEQ ID NO: 9618; SEQ ID NO: 9619; SEQ ID NO: 9620; SEQ ID NO: 9621, or a complement thereof.

In one embodiment, the H. pylori cytoplasmic polypeptide or a fragment thereof is an H. pylori polypeptide or a fragment thereof involved in energy metabolism encoded by a nucleic acid having a nucleotide sequence selected from the group consisting of SEQ ID NO: 1576; SEQ ID NO: 1577; SEQ ID NO: 1578; SEQ ID NO: 1579; SEQ ID NO: 1580; SEQ ID NO: 1581; SEQ ID NO: 1582; SEQ ID NO: 1583; SEQ ID NO: 1584; SEQ ID NO: 1585; SEQ ID NO: 1586; SEQ ID NO: 1587; SEQ ID NO: 1588; SEQ ID NO: 1589; SEQ ID NO: 1590; SEQ ID NO: 1591; SEQ ID NO: 1592; SEQ ID NO: 1593; SEQ ID NO: 1594; SEQ ID NO: 1595; SEQ ID NO: 1596; SEQ ID NO: 1597; SEQ ID NO: 1598; SEQ ID NO: 1599; SEQ ID NO: 1600; SEQ ID NO: 1601; SEQ ID NO: 1602; SEQ ID NO: 1603; SEQ ID NO: 1604; SEQ ID NO: 1605; SEQ ID NO: 1606; SEQ ID NO: 1607; SEQ ID NO: 1608; SEQ ID NO: 1609; SEQ ID NO: 1610; SEQ ID NO: 1611; SEQ ID NO: 1612; SEQ ID NO: 1613; SEQ ID NO: 1614; SEQ ID NO: 1615; SEQ ID NO: 1616; SEQ ID NO: 1617; SEQ ID NO: 1618; SEQ ID NO: 1619; SEQ ID NO: 1620; SEQ ID NO: 1621; SEQ ID NO: 1622; SEQ ID NO: 1623; SEQ ID NO: 1624; SEQ ID NO: 1625; SEQ ID NO: 1626; SEQ ID NO: 1627; SEQ ID NO: 1628; SEQ ID NO: 1629; SEQ ID NO: 1630; SEQ ID NO: 1631; SEQ ID NO: 1632; SEQ ID NO: 1633; SEQ ID NO: 1634; SEQ ID NO: 1635; SEQ ID NO: 1636; SEQ ID NO: 1637; SEQ ID NO: 1638; SEQ ID NO: 1639; SEQ ID NO: 1640; SEQ ID NO: 1641; SEQ ID NO: 1642; SEQ ID NO: 1643; SEQ ID NO: 1644; SEQ ID NO: 1645; SEQ ID NO: 1646; SEQ ID NO: 1647; SEQ ID NO: 1648; SEQ ID NO: 1649; SEQ ID NO: 1650; SEQ ID NO: 1651; SEQ ID NO: 1652; SEQ ID NO: 1653; SEQ ID NO: 1654; SEQ ID NO: 1655; SEQ ID NO: 1656; SEQ ID NO: 1657; SEQ ID NO: 1658; SEQ ID NO: 1659; SEQ ID NO: 1660; SEQ ID NO: 1661; SEQ ID NO: 1662; SEQ ID NO: 1663; SEQ ID NO: 1664; SEQ ID NO: 1665; SEQ ID NO: 1666; SEQ ID NO: 1667; SEQ ID NO: 1668; SEQ ID NO: 1669; SEQ ID NO: 1670; SEQ ID NO: 1671; SEQ ID NO: 1672; SEQ ID NO: 1673; SEQ ID NO: 1674; SEQ ID NO: 1675; SEQ ID NO: 1676; SEQ ID NO: 1677; SEQ ID NO: 1678; SEQ ID NO: 1679; SEQ ID NO: 1680; SEQ ID NO: 1681; SEQ ID NO: 1682; SEQ ID NO: 1683; SEQ ID NO: 1684; SEQ ID NO: 1685; SEQ ID NO: 1686; SEQ ID NO: 1687; SEQ ID NO: 1688; SEQ ID NO: 1689; SEQ ID NO: 1690; SEQ ID NO: 1691; SEQ ID NO: 1692; SEQ ID NO: 1693; SEQ ID NO: 1694; SEQ ID NO: 1695; SEQ ID NO: 1696; SEQ ID NO: 1697; SEQ ID NO: 1698; SEQ ID NO: 1699; SEQ ID NO: 1700; SEQ ID NO: 1701; SEQ ID NO: 1702; SEQ ID NO: 1703; SEQ ID NO: 1704; SEQ ID NO: 1705; SEQ ID NO: 1706; SEQ ID NO: 1707; SEQ ID NO: 1708; SEQ ID NO: 1709; SEQ ID NO: 1710; SEQ ID NO: 1711; SEQ ID NO: 1712; SEQ ID NO: 1713; SEQ ID NO: 1714; SEQ ID NO: 1715; SEQ ID NO: 1716; SEQ ID NO: 1717; SEQ ID NO: 1718; SEQ ID NO: 1719; SEQ ID NO: 1720; SEQ ID NO: 1721; SEQ ID NO: 1722; SEQ ID NO: 1723; SEQ ID NO: 1724; SEQ ID NO: 1725; and SEQ ID NO: 9593; SEQ ID NO: 9594, or a complement thereof.

In another embodiment, the H. pylori cytoplasmic polypeptide or a fragment thereof is an H. pylori polypeptide or a fragment thereof involved in amino acid metabolism encoded by a nucleic acid having a nucleotide sequence selected from the group consisting of SEQ ID NO: 1726; SEQ ID NO: 1727; SEQ ID NO: 1728; SEQ ID NO: 1729; SEQ ID NO: 1730; SEQ ID NO: 1731; SEQ ID NO: 1732; SEQ ID NO: 1733; SEQ ID NO: 1734; SEQ ID NO: 1735; SEQ ID NO: 1736; SEQ ID NO: 1737; SEQ ID NO: 1738; SEQ ID NO: 1739; SEQ ID NO: 1740; SEQ ID NO: 1741; SEQ ID NO: 1742; SEQ ID NO: 1743; SEQ ID NO: 1744; SEQ ID NO: 1745; SEQ ID NO: 1746; SEQ ID NO: 1747; SEQ ID NO: 1748; SEQ ID NO: 1749; SEQ ID NO: 1750; SEQ ID NO: 1751; SEQ ID NO: 1752; SEQ ID NO: 1753; SEQ ID NO: 1754; SEQ ID NO: 1755; SEQ ID NO: 1756; SEQ ID NO: 1757; SEQ ID NO: 1758; SEQ ID NO: 1759; SEQ ID NO: 1760; SEQ ID NO: 1761; SEQ ID NO: 1762; SEQ ID NO: 1763; SEQ ID NO: 1764; SEQ ID NO: 1765; SEQ ID NO: 1766; SEQ ID NO: 1767; SEQ ID NO: 1768; SEQ ID NO: 1769; SEQ ID NO: 1770; SEQ ID NO: 1771; SEQ ID NO: 1772; SEQ ID NO: 1773; SEQ ID NO: 1774; SEQ ID NO: 1775; SEQ ID NO: 1776; SEQ ID NO: 1777; SEQ ID NO: 1778; SEQ ID NO: 1779; SEQ ID NO: 1780; SEQ ID NO: 1781; SEQ ID NO: 1782; SEQ ID NO: 1783; SEQ ID NO: 1784; SEQ ID NO: 1785; SEQ ID NO: 1786; SEQ ID NO: 1787; SEQ ID NO: 1788; SEQ ID NO: 1789; SEQ ID NO: 1790; SEQ ID NO: 1791; SEQ ID NO: 1792; SEQ ID NO: 1793; SEQ ID NO: 1794; SEQ ID NO: 1795; SEQ ID NO: 1796; SEQ ID NO: 1797; SEQ ID NO: 1798; SEQ ID NO: 1799; SEQ ID NO: 1800; SEQ ID NO: 1801; SEQ ID NO: 1802; SEQ ID NO: 1803; SEQ ID NO: 1804; SEQ ID NO: 1805; SEQ ID NO: 1806; SEQ ID NO: 1807; SEQ ID NO: 1808; SEQ ID NO: 1809; SEQ ID NO: 1810; SEQ ID NO: 1811; SEQ ID NO: 1812; SEQ ID NO: 1813; SEQ ID NO: 1814; SEQ ID NO: 1815; SEQ ID NO: 1816; SEQ ID NO: 1817; SEQ ID NO: 1818; SEQ ID NO: 1819; SEQ ID NO: 1820; SEQ ID NO: 1821; SEQ ID NO: 1822; SEQ ID NO: 1823; SEQ ID NO: 1824; SEQ ID NO: 1825; SEQ ID NO: 1826; SEQ ID NO: 1827; SEQ ID NO: 1828; SEQ ID NO: 1829; SEQ ID NO: 1830; SEQ ID NO: 1831; SEQ ID NO: 1832; SEQ ID NO: 1833; SEQ ID NO: 1834; SEQ ID NO: 1835; SEQ ID NO: 1836; SEQ ID NO: 1837; SEQ ID NO: 1838; SEQ ID NO: 1839; SEQ ID NO: 1840; SEQ ID NO: 1841; SEQ ID NO: 1842; SEQ ID NO: 1843; SEQ ID NO: 1844; SEQ ID NO: 1845; SEQ ID NO: 1846; SEQ ID NO: 1847; SEQ ID NO: 1848; SEQ ID NO: 1849; SEQ ID NO: 1850; SEQ ID NO: 1851; SEQ ID NO: 1852; SEQ ID NO: 1853; SEQ ID NO: 1854; SEQ ID NO: 1855; SEQ ID NO: 1856; SEQ ID NO: 1857; SEQ ID NO: 1858; SEQ ID NO: 1859; SEQ ID NO: 1860; SEQ ID NO: 1861; SEQ ID NO: 1862; SEQ ID NO: 1863; SEQ ID NO: 1864; SEQ ID NO: 1865; SEQ ID NO: 1866; SEQ ID NO: 1867; SEQ ID NO: 1868 and SEQ ID NO: 9595; SEQ ID NO: 9596, or a complement thereof.

In another embodiment, the H. pylori cytoplasmic polypeptide or a fragment thereof is an H. pylori polypeptide or a fragment thereof involved in cofactor metabolism encoded by a nucleic acid having a nucleotide sequence selected from the group consisting of SEQ ID NO: 1869; SEQ ID NO: 1870; SEQ ID NO: 1871; SEQ ID NO: 1872; SEQ ID NO: 1873; SEQ ID NO: 1874; SEQ ID NO: 1875; SEQ ID NO: 1876; SEQ ID NO: 1877; SEQ ID NO: 1878; SEQ ID NO: 1879; SEQ ID NO: 1880; SEQ ID NO: 1881; SEQ ID NO: 1882; SEQ ID NO: 1883; SEQ ID NO: 1884; SEQ ID NO: 1885; SEQ ID NO: 1886; SEQ ID NO: 1887; SEQ ID NO: 1888; SEQ ID NO: 1889; SEQ ID NO: 1890; SEQ ID NO: 1891; SEQ ID NO: 1892; SEQ ID NO: 1893; SEQ ID NO: 1894; SEQ ID NO: 1895; SEQ ID NO: 1896; SEQ ID NO: 1897; SEQ ID NO: 1898; SEQ ID NO: 1899; SEQ ID NO: 1900; SEQ ID NO: 1901; SEQ ID NO: 1902; SEQ ID NO: 1903; SEQ ID NO: 1904; SEQ ID NO: 1905;-SEQ ID NO: 1906; SEQ ID NO: 1907; SEQ ID NO: 1908; SEQ ID NO: 1909; SEQ ID NO: 1910; SEQ ID NO: 1911; SEQ ID NO: 1912; SEQ ID NO: 1913; SEQ ID NO: 1914; SEQ ID NO: 1915; SEQ ID NO: 1916; SEQ ID NO: 1917; SEQ ID NO: 1918; SEQ ID NO: 1919; SEQ ID NO: 1920; SEQ ID NO: 1921; SEQ ID NO: 1922; SEQ ID NO: 1923; SEQ ID NO: 1924; SEQ ID NO: 1925; SEQ ID NO: 1926; SEQ ID NC. 927; SEQ ID NO: 1928; SEQ ID NO: 1929; SEQ ID NO: 1930; SEQ ID NO: 1931; SEQ ID NO: 1932; SEQ ID NO: 1933; SEQ ID NO: 1934; SEQ ID NO: 1935; SEQ ID NO: 1936; SEQ ID NO: 1937; SEQ ID NO: 1938; SEQ ID NO: 1939; SEQ ID NO: 1940; SEQ ID NO: 1941; SEQ ID NO: 1942; SEQ ID NO: 1943; SEQ ID NO: 1944; SEQ ID NO: 1945; SEQ ID NO: 1946; SEQ ID NO: 1947; SEQ ID NO: 1948; SEQ ID NO: 1949; SEQ ID NO: 1950; SEQ ID NO: 1951; SEQ ID NO: 1952; SEQ ID NO: 1953; SEQ ID NO: 1954; SEQ ID NO: 1955; SEQ ID NO: 1956; SEQ ID NO: 1957; SEQ ID NO: 1958; SEQ ID NO: 1959; SEQ ID NO: 1960; SEQ ID NO: 1961; SEQ ID NO: 1962; SEQ ID NO: 1963; SEQ ID NO: 1964; SEQ ID NO: 1965; SEQ ID NO: 1966; SEQ ID NO: 1967; SEQ ID NO: 1968; SEQ ID NO: 1969; SEQ ID NO: 1970; SEQ ID NO: 1971; SEQ ID NO: 1972; SEQ ID NO: 1973; SEQ ID NO: 1974; SEQ ID NO: 1975; SEQ ID NO: 1976; SEQ ID NO: 1977; SEQ ID NO: 1978; SEQ ID NO: 1979; SEQ ID NO: 1980; SEQ ID NO: 1981; SEQ ID NO: 1982; SEQ ID NO: 1983; SEQ ID NO: 1984; SEQ ID NO: 1985; SEQ ID NO: 1986; SEQ ID NO: 1987; SEQ ID NO: 1988; SEQ ID NO: 1989; SEQ ID NO: 1990; SEQ ID NO: 1991; SEQ ID NO: 1992; SEQ ID NO: 1993; SEQ ID NO: 1994; SEQ ID NO: 1995; SEQ ID NO: 1996; SEQ ID NO: 1997; SEQ ID NO: 1998; SEQ ID NO: 1999; SEQ ID NO: 2000; SEQ ID NO: 2001; SEQ ID NO: 2002; SEQ ID NO: 2003; SEQ ID NO: 2004; SEQ ID NO: 2005; and SEQ ID NO: 9597, or a complement thereof.

In another embodiment, the H. pylori cytoplasmic polypeptide or a fragment thereof is an H. pylori polypeptide or a fragment thereof involved in carbohydrate metabolism encoded by a nucleic acid having a nucleotide sequence selected from the group consisting of SEQ ID NO: 2006; SEQ ID NO: 2007; SEQ ID NO: 2008; SEQ ID NO: 2009; SEQ ID NO: 2010; SEQ ID NO: 2011; SEQ ID NO: 2012; SEQ ID NO: 2013; SEQ ID NO: 2014; SEQ ID NO: 2015; SEQ ID NO: 2016; SEQ ID NO: 2017; SEQ ID NO: 2018; SEQ ID NO: 2019; SEQ ID NO: 2020; SEQ ID NO: 2021; SEQ ID NO: 2022; SEQ ID NO: 2023; SEQ ID NO: 2024; SEQ ID NO: 2025; SEQ ID NO: 2026; SEQ ID NO: 2027; SEQ ID NO: 2028; SEQ ID NO: 2029; SEQ ID NO: 2030; SEQ ID NO: 2031; SEQ ID NO: 2032; SEQ ID NO: 2033; SEQ ID NO: 2034; SEQ ID NO: 2035; SEQ ID NO: 2036; SEQ ID NO: 2037; SEQ ID NO: 2038; SEQ ID NO: 2039; SEQ ID NO: 2040; SEQ ID NO: 2041; SEQ ID NO: 2042; SEQ ID NO: 2043; SEQ ID NO: 2044; SEQ ID NO: 2045; SEQ ID NO: 2046; SEQ ID NO: 2047; SEQ ID NO: 2048; SEQ ID NO: 2049; SEQ ID NO: 2050; SEQ ID NO: 2051; SEQ ID NO: 2052; SEQ ID NO: 2053; SEQ ID NO: 2054; SEQ ID NO: 2055; SEQ ID NO: 2056; SEQ ID NO: 2057; SEQ ID NO: 2058; SEQ ID NO: 2059; SEQ ID NO: 2060; SEQ ID NO: 2061; SEQ ID NO: 2062; SEQ ID NO: 2063; SEQ ID NO: 2064; SEQ ID NO: 2065; SEQ ID NO: 2066; SEQ ID NO: 2067; SEQ ID NO: 2068; SEQ ID NO: 2069; SEQ ID NO: 2070; SEQ ID NO: 2071; SEQ ID NO: 2072; SEQ ID NO: 2073; SEQ ID NO: 2074; SEQ ID NO: 2075; SEQ ID NO: 2076; and SEQ ID NO: 9598; SEQ ID NO: 9599, or a complement thereof.

In another embodiment, the H. pylori cytoplasmic polypeptide or a fragment thereof is an H. pylori polypeptide or a fragment thereof involved in nucleic acid metabolism encoded by a nucleic acid having a nucleotide sequence selected from the group consisting of SEQ ID NO: 2077; SEQ ID NO: 2078; SEQ ID NO: 2079; SEQ ID NO: 2080; SEQ ID NO: 2081; SEQ ID NO: 2082; SEQ ID NO: 2083; SEQ ID NO: 2084; SEQ ID NO: 2085; SEQ ID NO: 2086; SEQ ID NO: 2087; SEQ ID NO: 2088; SEQ ID NO: 2089; SEQ ID NO: 2090; SEQ ID NO: 2091; SEQ ID NO: 2092; SEQ ID NO: 2093; SEQ ID NO: 2094; SEQ ID NO: 2095; SEQ ID NO: 2096; SEQ ID NO: 2097; SEQ ID NO: 2098; SEQ ID NO: 2099; SEQ ID NO: 2100; SEQ ID NO: 2101; SEQ ID NO: 2102; SEQ ID NO: 2103; SEQ ID NO: 2104; SEQ ID NO: 2105; SEQ ID NO: 2106; SEQ ID NO: 2107; SEQ ID NO: 2108; SEQ ID NO: 2109; SEQ ID NO: 2110; SEQ ID NO: 2111; SEQ ID NO: 2112; SEQ ID NO: 2113; SEQ ID NO: 2114; SEQ ID NO:2115; SEQ ID NO:2116; SEQ ID NO:2117; SEQ ID NO:2118; SEQ ID NO: 2119; SEQ ID NO: 2120; SEQ ID NO: 2121; SEQ ID NO: 2122; SEQ ID NO: 2123; SEQ ID NO: 2124; SEQ ID NO: 2125; SEQ ID NO: 2126; SEQ ID NO: 2127; SEQ ID NO: 2128; SEQ ID NO: 2129; SEQ ID NO: 2130; SEQ ID NO: 2131; SEQ ID NO: 2132; SEQ ID NO: 2133; SEQ ID NO: 2134; SEQ ID NO: 2135; SEQ ID NO: 2136; SEQ ID NO: 2137; SEQ ID NO: 2138; SEQ ID NO: 2139; SEQ ID NO: 2140; SEQ ID NO: 2141; SEQ ID NO: 2142; SEQ ID NO: 2143; SEQ ID NO: 2144; SEQ ID NO: 2145; SEQ ID NO: 2146; SEQ ID NO: 2147; SEQ ID NO: 2148; SEQ ID NO: 2149; SEQ ID NO: 2150; SEQ ID NO: 2151; SEQ ID NO: 2152; SEQ ID NO: 2153; SEQ ID NO: 2154; SEQ ID NO: 2155; SEQ ID NO: 2156; SEQ ID NO: 2157; SEQ ID NO: 2158; SEQ ID NO:2159; SEQ ID NO:2160; SEQ ID NO:2161; SEQ ID NO: 2162; SEQ ID NO: 2163; SEQ ID NO: 2164; SEQ ID NO: 2165; SEQ ID NO: 2166; SEQ ID NO: 2167; SEQ ID NO: 2168; SEQ ID NO: 2169; SEQ ID NO: 2170; SEQ ID NO: 2171; SEQ ID NO: 2172; SEQ ID NO: 2173; SEQ ID NO: 2174; SEQ ID NO: 2175; SEQ ID NO: 2176; SEQ ID NO: 2177; SEQ ID NO: 2178; SEQ ID NO: 2179; SEQ ID NO: 2180; SEQ ID NO: 2181; SEQ ID NO: 2182; and SEQ ID NO: 9600, or a complement thereof.

In another embodiment, the H. pylori cytoplasmic polypeptide or a fragment thereof is an H. pylori polypeptide or a fragment thereof involved in lipid metabolism encoded by a nucleic acid having a nucleotide sequence selected from the group consisting of SEQ ID NO: 2183; SEQ ID NO: 2184; SEQ ID NO: 2185; SEQ ID NO: 2186; SEQ ID NO: 2187; SEQ ID NO: 2188; SEQ ID NO: 2189; SEQ ID NO: 2190; SEQ ID NO: 2191; SEQ ID NO: 2192; SEQ ID NO: 2193; SEQ ID NO: 2194; SEQ ID NO 2195; SEQ ID NO: 2196; SEQ ID NO: 2197; SEQ ID NO: 2198; SEQ ID NO: 2199; SEQ ID NO: 2200; SEQ ID NO: 2201; SEQ ID NO: 2202; SEQ ID NO: 2203; SEQ ID NO: 2204; SEQ ID NO: 2205; SEQ ID NO: 2206; SEQ ID NO: 2207; SEQ ID NO: 2208; SEQ ID NO: 2209; SEQ ID NO: 2210; SEQ ID NO: 2211; SEQ ID NO: 2212; SEQ ID NO: 2213; SEQ ID NO: 2214; SEQ ID NO: 2215; SEQ ID NO: 2216; SEQ ID NO: 2217; SEQ ID NO: 2218; and SEQ ID NO: 9601; SEQ ID NO: 9602, or a complement thereof.

In another embodiment, the H. pylori cytoplasmic polypeptide or a fragment thereof is an H. pylori polypeptide or a fragment thereof involved in mRNA translation and ribosome biogenesis encoded by a nucleic acid having a nucleotide sequence selected from the group consisting of SEQ ID NO: 2219; SEQ ID NO: 2220; SEQ ID NO: 2221; SEQ ID NO: 2222; SEQ ID NO: 2223; SEQ ID NO: 2224; SEQ ID NO: 2225; SEQ ID NO: 2226; SEQ ID NO: 2227; SEQ ID NO: 2228; SEQ ID NO: 2229; SEQ ID NO: 2230; SEQ ID NO: 2231; SEQ ID NO: 2232; SEQ ID NO: 2233; SEQ ID NO: 2234; SEQ ID NO: 2235; SEQ ID NO: 2236; SEQ ID NO: 2237; SEQ ID NO: 2238; SEQ ID NO: 2239; SEQ ID NO: 2240; SEQ ID NO: 2241; SEQ ID NO: 2242; SEQ ID NO: 2243; SEQ ID NO: 2244; SEQ ID NO: 2245; SEQ ID NO: 2246; SEQ ID NO: 2247; SEQ ID NO: 2248; SEQ ID NO: 2249; SEQ ID NO: 2250; SEQ ID NO: 2251; SEQ ID NO: 2252; SEQ ID NO: 2253; SEQ ID NO: 2254; SEQ ID NO: 2255; SEQ ID NO: 2256; SEQ ID NO: 2257; SEQ ID NO: 2258; SEQ ID NO: 2259; SEQ ID NO: 2260; SEQ ID NO: 2261; SEQ ID NO: 2262; SEQ ID NO: 2263; SEQ ID NO: 2264; SEQ ID NO: 2265; SEQ ID NO: 2266; SEQ ID NO: 2267; SEQ ID NO: 2268; SEQ ID NO: 2269; SEQ ID NO: 2270; SEQ ID NO: 2271; SEQ ID NO: 2272; SEQ ID NO: 2273; SEQ ID NO: 2274; SEQ ID NO: 2275; SEQ ID NO: 2276; SEQ ID NO: 2277; SEQ ID NO: 2278; SEQ ID NO: 2279; SEQ ID NO: 2280; SEQ ID NO: 2281; SEQ ID NO: 2282; SEQ ID NO: 2283; SEQ ID NO: 2284; SEQ ID NO: 2285; SEQ ID NO: 2286; SEQ ID NO: 2287; SEQ ID NO: 2288; SEQ ID NO: 2289; SEQ ID NO: 2290; SEQ ID NO: 2291; SEQ ID NO: 2292; SEQ ID NO: 2293; SEQ ID NO: 2294; SEQ ID NO: 2295; SEQ ID NO: 2296; SEQ ID NO: 2297; SEQ ID NO: 2298; SEQ ID NO: 2299; SEQ ID NO: 2300; SEQ ID NO: 2301; SEQ ID NO: 2302; SEQ ID NO: 2303; SEQ ID NO: 2304; SEQ ID NO: 2305; SEQ ID NO: 2306; SEQ ID NO: 2307; SEQ ID NO: 2308; SEQ ID NO: 2309; SEQ ID NO: 2310; SEQ ID NO: 2311; SEQ ID NO: 2312; SEQ ID NO: 2313; SEQ ID NO: 2314; SEQ ID NO: 2315; SEQ ID NO: 2316; SEQ ID NO: 2317; SEQ ID NO: 2318; SEQ ID NO: 2319; SEQ ID NO: 2320; SEQ ID NO: 2321; SEQ ID NO: 2322; SEQ ID NO: 2323; SEQ ID NO: 2324; SEQ ID NO: 2325; SEQ ID NO: 2326; SEQ ID NO: 2327; SEQ ID NO: 2328; SEQ ID NO: 2329; SEQ ID NO: 2330; SEQ ID NO: 2331; SEQ ID NO: 2332; SEQ ID NO: 2333; SEQ ID NO: 2334; SEQ ID NO: 2335; SEQ ID NO: 2336; SEQ ID NO: 2337; SEQ ID NO: 2338; SEQ ID NO: 2339; SEQ ID NO: 2340; SEQ ID NO: 2341; SEQ ID NO: 2342; SEQ ID NO: 2343; SEQ ID NO: 2344; SEQ ID NO: 2345; SEQ ID NO: 2346; SEQ ID NO: 2347; SEQ ID NO: 2348; SEQ ID NO: 2349; SEQ ID NO: 2350; SEQ ID NO: 2351; SEQ ID NO: 2352; SEQ ID NO: 2353; SEQ ID NO: 2354; SEQ ID NO: 2355; SEQ ID NO: 2356; SEQ ID NO: 2357; SEQ ID NO: 2358; SEQ ID NO: 2359; SEQ ID NO: 2360; SEQ ID NO: 2361; SEQ ID NO: 2362; SEQ ID NO: 2363; SEQ ID NO: 2364; SEQ ID NO: 2365; SEQ ID NO: 2366; SEQ ID NO: 2367; SEQ ID NO: 2368; SEQ ID NO: 2369; SEQ ID NO: 2370; SEQ ID NO: 2371; SEQ ID NO: 2372; SEQ ID NO: 2373; SEQ ID NO: 2374; SEQ ID NO: 2375; SEQ ID NO: 2376; SEQ ID NO: 2377; SEQ ID NO: 2378; SEQ ID NO: 2379; SEQ ID NO: 2380; SEQ ID NO: 2381; SEQ ID NO: 2382; SEQ ID NO: 2383; SEQ ID NO: 2384; SEQ ID NO: 2385; SEQ ID NO: 2386; SEQ ID NO: 2387; SEQ ID NO: 2388; SEQ ID NO: 2389; SEQ ID NO: 2390; SEQ ID NO: 2391; SEQ ID NO: 2392; SEQ ID NO: 2393; SEQ ID NO: 2394; SEQ ID NO: 2395; SEQ ID NO: 2396; SEQ ID NO: 2397; SEQ ID NO: 2398; SEQ ID NO: 2399; SEQ ID NO: 2400; SEQ ID NO: 2401; SEQ ID NO: 2402; SEQ ID NO: 2403; SEQ ID NO: 2404; SEQ ID NO: 2405; SEQ ID NO: 2406; SEQ. ID NO: 2407; SEQ ID NO: 2408; SEQ ID NO: 2409; SEQ ID NO: 2410; SEQ ID NO: 2411; SEQ ID NO: 2412; SEQ ID NO: 2413; SEQ ID NO: 2414; SEQ ID NO: 2415; SEQ ID NO: 2416; SEQ ID NO: 2417; SEQ ID NO: 2418; SEQ ID NO: 2419; SEQ ID NO: 2420; SEQ ID NO: 2421; SEQ ID NO: 2422; SEQ ID NO: 2423; SEQ ID NO: 2424; SEQ ID NO: 2425; SEQ ID NO: 2426; SEQ ID NO: 2427; SEQ ID NO: 2428; SEQ ID NO: 2429; SEQ ID NO: 2430; SEQ ID NO: 2431; SEQ ID NO: 2432; SEQ ID NO: 2433; SEQ ID NO: 2434; SEQ ID NO: 2435; SEQ ID NO: 2436; SEQ ID NO: 2437; SEQ ID NO: 2438 and SEQ ID NO: 9603; SEQ ID NO: 9604, or a complement thereof.

In another embodiment, the H. pylori cytoplasmic polypeptide or a fragment thereof is an H. pylori polypeptide or a fragment thereof involved in genome replication, transcription, recombination and repair encoded by a nucleic acid having a nucleotide sequence selected from the group consisting of SEQ ID NO: 2439; SEQ ID NO: 2440; SEQ ID NO: 2441; SEQ ID NO: 2442; SEQ ID NO: 2443; SEQ ID NO: 2444; SEQ ID NO: 2445; SEQ ID NO: 2446; SEQ ID NO: 2447; SEQ ID NO: 2448; SEQ ID NO: 2449; SEQ ID NO: 2450; SEQ ID NO: 2451; SEQ ID NO: 2452; SEQ ID NO: 2453; SEQ ID NO: 2454; SEQ ID NO: 2455; SEQ ID NO: 2456; SEQ ID NO: 2457; SEQ ID NO: 2458; SEQ ID NO: 2459; SEQ ID NO: 2460; SEQ ID NO: 2461; SEQ ID NO: 2462; SEQ ID NO: 2463; SEQ ID NO: 2464; SEQ ID NO: 2465; SEQ ID NO: 2466; SEQ ID NO: 2467; SEQ ID NO: 2468; SEQ ID NO: 2469; SEQ ID NO: 2470; SEQ ID NO: 2471; SEQ ID NO: 2472; SEQ ID NO: 2473; SEQ ID NO: 2474; SEQ ID NO: 2475; SEQ ID NO: 2476; SEQ ID NO: 2477; SEQ ID NO: 2478; SEQ ID NO: 2479; SEQ ID NO: 2480; SEQ ID NO: 2481; SEQ ID NO: 2482; SEQ ID NO: 2483; SEQ ID NO: 2484; SEQ ID NO: 2485; SEQ ID NO: 2486; SEQ ID NO: 2487; SEQ ID NO: 2488; SEQ ID NO: 2489; SEQ ID NO: 2490; SEQ ID NO: 2491; SEQ ID NO: 2492; SEQ ID NO: 2493; SEQ ID NO: 2494; SEQ ID NO: 2495; SEQ ID NO: 2496; SEQ ID NO: 2497; SEQ ID NO: 2498; SEQ ID NO: 2499; SEQ ID NO: 2500; SEQ ID NO: 2501; SEQ ID NO: 2502; SEQ ID NO: 2503; SEQ ID NO: 2504; SEQ ID NO: 2505; SEQ ID NO: 2506; SEQ ID NO: 2507; SEQ ID NO: 2508; SEQ ID NO: 2509; SEQ ID NO: 2510; SEQ ID NO: 2511; SEQ ID NO: 2512; SEQ ID NO: 2513; SEQ ID NO: 2514; SEQ ID NO: 2515; SEQ ID NO: 2516; SEQ ID NO: 2517; SEQ ID NO: 2518; SEQ ID NO: 2519; SEQ ID NO: 2520; SEQ ID NO: 2521; SEQ ID NO: 2522; SEQ ID NO: 2523; SEQ ID NO: 2524; SEQ ID NO: 2525; SEQ ID NO: 2526; SEQ ID NO: 2527; SEQ ID NO: 2528; SEQ ID NO: 2529; SEQ ID NO: 2530; SEQ ID NO: 2531; SEQ ID NO: 2532; SEQ ID NO: 2533; SEQ ID NO: 2534; SEQ ID NO: 2535; SEQ ID NO: 2536; SEQ ID NO: 2537; SEQ ID NO: 2538; SEQ ID NO: 2539; SEQ ID NO: 2540; SEQ ID NO: 2541; SEQ ID NO: 2542; SEQ ID NO: 2543; SEQ ID NO: 2544; SEQ ID NO: 2545; SEQ ID NO: 2546; SEQ ID NO: 2547; SEQ ID NO: 2548; SEQ ID NO: 2549; SEQ ID NO: 2550; SEQ ID NO: 2551; SEQ ID NO: 2552; SEQ ID NO: 2553; SEQ ID NO: 2554; SEQ ID NO: 2555; SEQ ID NO: 2556; SEQ ID NO: 2557; SEQ ID NO: 2558; SEQ ID NO: 2559; SEQ ID NO: 2560; SEQ ID NO: 2561; SEQ ID NO: 2562; SEQ ID NO: 2563; SEQ ID NO: 2564; SEQ ID NO: 2565; SEQ ID NO: 2566; SEQ ID NO: 2567; SEQ ID NO: 2568; SEQ ID NO: 2569; SEQ ID NO: 2570; SEQ ID NO: 2571; SEQ ID NO: 2572; SEQ ID NO: 2573; SEQ ID NO: 2574; SEQ ID NO: 2575; SEQ ID NO: 2576; SEQ ID NO: 2577; SEQ ID NO: 2578; SEQ ID NO: 2579; SEQ ID NO: 2580; SEQ ID NO: 2581; SEQ ID NO: 2582; SEQ ID NO: 2583; SEQ ID NO: 2584; SEQ ID NO: 2585; SEQ ID NO: 2586; SEQ ID NO: 2587; SEQ ID NO: 2588; SEQ ID NO: 2589; SEQ ID NO: 2590; SEQ ID NO: 2591; SEQ ID NO: 2592; SEQ ID NO: 2593; SEQ ID NO: 2594; SEQ ID NO: 2595; SEQ ID NO: 2596; SEQ ID NO: 2597; SEQ ID NO: 2598; SEQ ID NO: 2599; SEQ ID NO: 2600; SEQ ID NO: 2601; SEQ ID NO: 2602; SEQ ID NO: 2603; SEQ ID NO: 2604; SEQ ID NO: 2605; SEQ ID NO: 2606; SEQ ID NO: 2607; SEQ ID NO: 2608; SEQ ID NO: 2609; SEQ ID NO: 2610; SEQ ID NO: 2611; SEQ ID NO: 2612; SEQ ID NO: 2613; SEQ ID NO: 2614; SEQ ID NO: 2615; SEQ ID NO: 2616; SEQ ID NO: 2617; SEQ ID NO: 2618; SEQ ID NO: 2619; SEQ ID NO: 2620; SEQ ID NO: 2621; SEQ ID NO: 2622; SEQ ID NO: 2623; SEQ ID NO: 2624; SEQ ID NO: 2625; SEQ ID NO: 2626; SEQ ID NO: 2627; SEQ ID NO: 2628; SEQ ID NO: 2629; SEQ ID NO: 2630; SEQ ID NO: 2631; SEQ ID NO: 2632; SEQ ID NO: 2633; SEQ ID NO: 2634; SEQ ID NO: 2635; SEQ ID NO: 2636; SEQ ID NO: 2637; SEQ ID NO: 2638; SEQ ID NO: 2639; SEQ ID NO: 2640; SEQ ID NO:2641; SEQ ID NO: 2642; SEQ ID NO: 2643; SEQ ID NO: 2644; SEQ ID NO: 2645; SEQ ID NO: 2646; SEQ ID NO: 2647; SEQ ID NO: 2648; SEQ ID NO: 2649; SEQ ID NO: 2650; SEQ ID NO: 2651; SEQ ID NO: 2652; SEQ ID NO: 2653; SEQ ID NO: 2654; SEQ ID NO: 2655; SEQ ID NO: 2656; SEQ ID NO: 2657; SEQ ID NO: 2658; SEQ ID NO: 2659; SEQ ID NO: 2660; SEQ ID NO: 2661; SEQ ID NO: 2662; SEQ ID NO: 2663; SEQ ID NO: 2664; SEQ ID NO: 2665; SEQ ID NO: 2666; SEQ ID NO: 2667; SEQ ID NO: 2668; SEQ ID NO: 2669; SEQ ID NO: 2670; SEQ ID NO: 2671; SEQ ID NO: 2672; SEQ ID NO: 2673; SEQ ID NO: 2674; SEQ ID NO: 2675; SEQ ID NO: 2676; SEQ ID NO: 2677; SEQ ID NO: 2678; SEQ ID NO: 2679; SEQ ID NO: 2680; SEQ ID NO: 2681; SEQ ID NO: 2682; SEQ ID NO: 2683; SEQ ID NO: 2684; SEQ ID NO: 2685; SEQ ID NO: 2686; SEQ ID NO: 2687; SEQ ID NO: 2688; SEQ ID NO: 2689; SEQ ID NO: 2690; SEQ ID NO: 2691; SEQ ID NO: 2692; SEQ ID NO: 2693; SEQ ID NO: 2694; SEQ ID NO: 2695; SEQ ID NO: 2696; SEQ ID NO: 2697; SEQ ID NO: 2698; SEQ ID NO: 2699; SEQ ID NO: 2700; SEQ ID NO: 2701; SEQ ID NO: 2702; SEQ ID NO: 2703; SEQ ID NO: 2704; SEQ ID NO: 2705; SEQ ID NO: 2706; SEQ ID NO: 2707; SEQ ID NO: 2708; SEQ ID NO: 2709; SEQ ID NO: 2710; SEQ ID NO: 2711; SEQ ID NO: 2712; SEQ ID NO: 2713; SEQ ID NO: 2714; SEQ ID NO: 2715; SEQ ID NO: 2716; SEQ ID NO: 2717; SEQ ID NO: 2718; SEQ ID NO: 2719; SEQ ID NO: 2720; SEQ ID NO: 2721; SEQ ID NO: 2722; SEQ ID NO: 2723; SEQ ID NO: 2724; SEQ ID NO: 2725; SEQ ID NO: 2726; SEQ ID NO: 2727; SEQ ID NO: 2728; SEQ ID NO: 2729; SEQ ID NO: 2730; SEQ ID NO: 2731; SEQ ID NO: 2732; SEQ ID NO: 2733; SEQ ID NO: 2734; SEQ ID NO: 2735; SEQ ID NO: 2736; SEQ ID NO: 2737; SEQ ID NO: 2738; SEQ ID NO: 2739; SEQ ID NO: 2740; SEQ ID NO: 2741; SEQ ID NO: 2742; SEQ ID NO: 2743; SEQ ID NO: 2744; SEQ ID NO: 2745; SEQ ID NO: 2746; SEQ ID NO: 2747; SEQ ID NO: 2748; SEQ ID NO: 2749; SEQ ID NO: 2750; SEQ ID NO: 2751; SEQ ID NO: 2752; SEQ ID NO: 2753; SEQ ID NO: 2754; SEQ ID NO: 2755; SEQ ID NO: 2756; SEQ ID NO: 2757; SEQ ID NO: 2758; SEQ ID NO: 2759; SEQ ID NO: 2760; SEQ ID NO: 2761; SEQ ID NO: 2762; SEQ ID NO: 2763; SEQ ID NO: 2764; SEQ ID NO: 2765; SEQ ID NO: 2766; SEQ ID NO: 2767; SEQ ID NO: 2768; SEQ ID NO: 2769; SEQ ID NO: 2770; SEQ ID NO: 2771; SEQ ID NO: 2772; SEQ ID NO: 2773; SEQ ID NO: 2774; SEQ ID NO: 2775; SEQ ID NO: 2776; SEQ ID NO: 2777; SEQ ID NO: 2778; SEQ ID NO: 2779; SEQ ID NO: 2780; SEQ ID NO: 2781; SEQ ID NO: 2782; SEQ ID NO: 2783; SEQ ID NO: 2784; SEQ ID NO: 2785; SEQ ID NO: 2786; SEQ ID NO: 2787; SEQ ID NO: 2788; SEQ ID NO: 2789; SEQ ID NO: 2790; SEQ ID NO: 2791; SEQ ID NO: 2792; SEQ ID NO: 2793; SEQ ID NO: 2794; SEQ ID NO: 2795; SEQ ID NO: 2796; SEQ ID NO: 2797; SEQ ID NO: 2798; SEQ ID NO: 2799; SEQ ID NO: 2800; SEQ ID NO: 2801; SEQ ID NO: 2802; SEQ ID NO: 2803; SEQ ID NO: 2804; SEQ ID NO: 2805; SEQ ID NO: 2806; SEQ ID NO: 2807; and SEQ ID NO: 9605; SEQ ID NO: 9606; SEQ ID NO: 9607 and SEQ ID NO: 9608, or a complement thereof.

In another embodiment, the H. pylori cytoplasmic polypeptide or a fragment thereof is an H. pylori polypeptide or a fragment thereof involved in secretion and adhesion encoded by a nucleic acid having a nucleotide sequence selected from the group consisting of SEQ ID NO: 2808; SEQ ID NO: 2809; SEQ ID NO: 2810; SEQ ID NO: 2811; SEQ ID NO: 2812; SEQ ID NO: 2813; SEQ ID NO: 2814; SEQ ID NO: 2815; SEQ ID NO: 2816; SEQ ID NO: 2817; SEQ ID NO: 2818; SEQ ID NO: 2819; SEQ ID NO: 2820; SEQ ID NO: 2821; SEQ ID NO: 2822; SEQ ID NO: 2823 and SEQ ID NO: 2824 or a complement thereof.

In another embodiment, the H. pylori cytoplasmic polypeptide or a fragment thereof is an H. pylori polypeptide or a fragment thereof involved in outer membrane and cell wall biogenesis encoded by a nucleic acid having a nucleotide sequence selected from the group consisting of SEQ ID NO: 2825; SEQ ID NO: 2826; SEQ ID NO: 2827; SEQ ID NO: 2828; SEQ ID NO: 2829; SEQ ID NO: 2830; SEQ ID NO: 2831; SEQ ID NO: 2832; SEQ ID NO: 2833; SEQ ID NO: 2834; SEQ ID NO:2835; SEQ ID NO: 2836; SEQ ID NO: 2837; SEQ ID NO: 2838; SEQ ID NO: 2839; SEQ ID NO: 2840; SEQ ID NO: 2841; SEQ ID NO: 2842; SEQ ID NO: 2843; SEQ ID NO: 2844; SEQ ID NO: 2845; SEQ ID NO: 2846; SEQ ID NO: 2847; SEQ ID NO: 2848; SEQ ID NO: 2849; SEQ ID NO: 2850; SEQ ID NO: 2851; SEQ ID NO: 2852; SEQ ID NO: 2853; SEQ ID NO: 2854; SEQ ID NO: 2855; SEQ ID NO: 2856; SEQ ID NO: 2857; SEQ ID NO: 2858; SEQ ID NO: 2859; SEQ ID NO: 2860; SEQ ID NO: 2861; SEQ ID NO: 2862; SEQ ID NO: 2863; SEQ ID NO: 2864; SEQ ID NO: 2865; SEQ ID NO: 2866; SEQ ID NO: 2867; SEQ ID NO: 2868; SEQ ID NO: 2869; SEQ ID NO: 2870; SEQ ID NO: 2871; SEQ ID NO: 2872; SEQ ID NO: 2873; SEQ ID NO: 2874; SEQ ID NO: 2875; SEQ ID NO: 2876; SEQ ID NO: 2877; and SEQ ID NO: 9609 and SEQ ID NO: 9610, or a complement thereof.

In another embodiment, the H. pylori cytoplasmic polypeptide or a fragment thereof is an H. pylori polypeptide or a fragment thereof involved in protein folding and stabilization encoded by a nucleic acid having a nucleotide sequence selected from the group consisting of SEQ ID NO: 2878; SEQ ID NO: 2879; SEQ ID NO: 2880; SEQ ID NO: 2881; SEQ ID NO: 2882; SEQ ID NO: 2883; SEQ ID NO: 2884; SEQ ID NO: 2885; SEQ ID NO: 2886; SEQ ID NO: 2887; SEQ ID NO: 2888; SEQ ID NO: 2889; SEQ ID NO: 2890; SEQ ID NO: 2891; SEQ ID NO: 2892; SEQ ID NO: 2893; SEQ ID NO: 2894; SEQ ID NO: 2895; SEQ ID NO: 2896; SEQ ID NO: 2897; SEQ ID NO: 2898; SEQ ID NO: 2899; SEQ ID NO: 2900; SEQ ID NO: 2901; SEQ ID NO: 2902; SEQ ID NO: 2903; SEQ ID NO: 2904; SEQ ID NO: 2905; SEQ ID NO: 2906; SEQ ID NO: 2907; SEQ ID NO: 2908; SEQ ID NO: 2909; SEQ ID NO: 2910; SEQ ID NO: 2911; SEQ ID NO: 2912; SEQ ID NO: 2913; SEQ ID NO: 2914; SEQ ID NO: 2915; SEQ ID NO: 2916; SEQ ID NO: 2917; SEQ ID NO: 2918; and SEQ ID NO: 9611 and SEQ ID NO: 9612, or a complement thereof.

Particularly preferred is an isolated nucleic acid having a nucleotide sequence encoding an H. pylori secreted polypeptide or a fragment thereof, the nucleic acid selected from the group consisting of SEQ ID NO: 4016; SEQ ID NO: 4017; SEQ ID NO: 4018; SEQ ID NO: 4019; SEQ ID NO: 4020; SEQ ID NO: 4021; SEQ ID NO: 4022; SEQ ID NO: 4023; SEQ ID NO: 4024; SEQ ID NO: 4025; SEQ ID NO: 4026; SEQ ID NO: 4027; SEQ ID NO: 4028; SEQ ID NO: 4029; SEQ ID NO: 4030; SEQ ID NO: 4031; SEQ ID NO: 4032; SEQ ID NO: 4033; SEQ ID NO: 4034; SEQ ID NO: 4035; SEQ ID NO: 4036; SEQ ID NO: 4037; SEQ ID NO: 4038; SEQ ID NO: 4039; SEQ ID NO: 4040; SEQ ID NO: 4041; SEQ ID NO: 4042; SEQ ID NO: 4043; SEQ ID NO: 4044; SEQ ID NO: 4045; SEQ ID NO: 4046; SEQ ID NO: 4047; SEQ ID NO: 4048; SEQ ID NO: 4049; SEQ ID NO: 4050; SEQ ID NO: 4051; SEQ ID NO: 4052; SEQ ID NO: 4053; SEQ ID NO: 4054; SEQ ID NO: 4055; SEQ ID NO: 4056; SEQ ID NO: 4057; SEQ ID NO: 4058; SEQ ID NO: 4059; SEQ ID NO: 4060; SEQ ID NO: 4061; SEQ ID NO: 4062; SEQ ID NO: 4063; SEQ ID NO: 4064; SEQ ID NO: 4065; SEQ ID NO: 4066; SEQ ID NO: 4067; SEQ ID NO: 4068; SEQ ID NO: 4069; SEQ ID NO: 4070; SEQ ID NO: 4071; SEQ ID NO: 4072; SEQ ID NO: 4073; SEQ ID NO: 4074; SEQ ID NO: 4075; SEQ ID NO: 4076; SEQ ID NO: 4077; SEQ ID NO: 4078; SEQ ID NO: 4079; SEQ ID NO: 4080; SEQ ID NO: 4081; SEQ ID NO: 4082; SEQ ID NO: 4083; SEQ ID NO: 4084; SEQ ID NO: 4085; SEQ ID NO: 4086; SEQ ID NO: 4087; SEQ ID NO: 4088; SEQ ID NO: 4089; SEQ ID NO: 4090; SEQ ID NO: 4091; SEQ ID NO: 4092; SEQ ID NO: 4093; SEQ ID NO: 4094; SEQ ID NO: 4095; SEQ ID NO: 4096; SEQ ID NO: 4097; SEQ ID NO: 4098; SEQ ID NO: 4099; SEQ ID NO: 4100; SEQ ID NO: 4101; SEQ ID NO: 4102; SEQ ID NO: 4103; SEQ ID NO: 4104; SEQ ID NO: 4105; SEQ ID NO: 4106; SEQ ID NO: 4107; SEQ ID NO: 4108; SEQ ID NO: 4109; SEQ ID NO: 4110; SEQ ID NO: 4111; SEQ ID NO: 4112; SEQ ID NO: 4113; SEQ ID NO: 4114; SEQ ID NO: 4115; SEQ ID NO: 4116; SEQ ID NO: 4117; SEQ ID NO: 4118; SEQ ID NO: 4119; SEQ ID NO: 4120; SEQ ID NO: 4121; SEQ ID NO: 4122; SEQ ID NO: 4123; SEQ ID NO: 4124; SEQ ID NO: 4125; SEQ ID NO: 4126; SEQ ID NO: 4127; SEQ ID NO: 4128; SEQ ID NO: 4129; SEQ ID NO: 4130; SEQ ID NO: 4131; SEQ ID NO: 4132; SEQ ID NO: 4133; SEQ ID NO: 4134; SEQ ID NO: 4135; SEQ ID NO: 4136; SEQ ID NO: 4137; SEQ ID NO: 4138; SEQ ID NO: 4139; SEQ ID NO: 4140; SEQ ID NO: 4141; SEQ ID NO: 4142; SEQ ID NO: 4143; SEQ ID NO: 4144; SEQ ID NO: 4145; SEQ ID NO: 4146; SEQ ID NO: 4147; SEQ ID NO: 4148; SEQ ID NO: 4149; SEQ ID NO: 4150; SEQ ID NO: 4151; SEQ ID NO: 4152; SEQ ID NO: 4153; SEQ ID NO: 4154; SEQ ID NO: 4155; SEQ ID NO: 4156; SEQ ID NO: 4157; SEQ ID NO: 4158; SEQ ID NO: 4159; SEQ ID NO: 4160; SEQ ID NO: 4161; SEQ ID NO: 4162; SEQ ID NO: 4163; SEQ ID NO: 4164; SEQ ID NO: 4165; SEQ ID NO: 4166; SEQ ID NO: 4167; SEQ ID NO: 4168; SEQ ID NO: 4169; SEQ ID NO: 4170; SEQ ID NO: 4171; SEQ ID NO: 4172; SEQ ID NO: 4173; SEQ ID NO: 4174; SEQ ID NO: 4175; SEQ ID NO: 4176; SEQ ID NO: 4177; SEQ ID NO: 4178; SEQ ID NO: 4179; SEQ ID NO: 4180; SEQ ID NO: 4181; SEQ ID NO: 4182; SEQ ID NO: 4183; SEQ ID NO: 4184; SEQ ID NO: 4185; SEQ ID NO: 4186; SEQ ID NO: 4187; SEQ ID NO: 4188; SEQ ID NO: 4189; SEQ ID NO: 4190; SEQ ID NO: 4191; SEQ ID NO: 4192; SEQ ID NO: 4193; SEQ ID NO: 4194; SEQ ID NO: 4195; SEQ ID NO: 4196; SEQ ID NO: 4197; SEQ ID NO: 4198; SEQ ID NO: 4199; SEQ ID NO: 4200; SEQ ID NO: 4201; SEQ ID NO: 4202; SEQ ID NO: 4203; SEQ ID NO: 4204; SEQ ID NO: 4205; SEQ ID NO: 4206; SEQ ID NO: 4207; SEQ ID NO: 4208; SEQ ID NO: 4209; SEQ ID NO: 4210; SEQ ID NO: 4211; SEQ ID NO: 4212; SEQ ID NO: 4213; SEQ ID NO: 4214; SEQ ID NO: 4215; SEQ ID NO: 4216; SEQ ID NO: 4217; SEQ ID NO: 4218; SEQ ID NO: 4219; SEQ ID NO: 4220; SEQ ID NO: 4221; SEQ ID NO: 4222; SEQ ID NO: 4223; SEQ ID NO: 4224; SEQ ID NO: 4225; SEQ ID NO: 4226; SEQ ID NO: 4227; SEQ ID NO: 4228; SEQ ID NO: 4229; SEQ ID NO: 4230; SEQ ID NO: 4231; SEQ ID NO: 4232; SEQ ID NO: 4233; SEQ ID NO: 4234; SEQ ID NO: 4235; SEQ ID NO: 4236; SEQ ID NO: 4237; SEQ ID NO: 4238; SEQ ID NO: 4239; SEQ ID NO: 4240; SEQ ID NO: 4241; SEQ ID NO: 4242; SEQ ID NO: 4243; SEQ ID NO: 4244; SEQ ID NO: 4245; SEQ ID NO: 4246; SEQ ID NO: 4247; SEQ ID NO: 4248; SEQ ID NO: 4249; SEQ ID NO: 4250; SEQ ID NO: 4251; SEQ ID NO: 4252; SEQ ID NO: 4253; SEQ ID NO: 4254; SEQ ID NO: 4255; SEQ ID NO: 4256; SEQ ID NO: 4257; SEQ ID NO: 4258; SEQ ID NO: 4259; SEQ ID NO: 4260; SEQ ID NO: 4261; SEQ ID NO: 4262; SEQ ID NO: 4263; SEQ ID NO: 4264; SEQ ID NO: 4265; SEQ ID NO: 4266; SEQ ID NO: 4267; SEQ ID NO: 4268; SEQ ID NO: 4269; SEQ ID NO: 4270; SEQ ID NO: 4271; SEQ ID NO: 4272; SEQ ID NO: 4273; SEQ ID NO: 4274; SEQ ID NO: 4275; SEQ ID NO: 4276; SEQ ID NO: 4277; SEQ ID NO: 4278; SEQ ID NO: 4279; SEQ ID NO: 4280; SEQ ID NO: 4281; SEQ ID NO: 4282; SEQ ID NO: 4283; SEQ ID NO: 4284; SEQ ID NO: 4285; SEQ ID NO: 4286; SEQ ID NO: 4287; SEQ ID NO: 4288; SEQ ID NO: 4289; SEQ ID NO: 4290; SEQ ID NO: 4291; SEQ ID NO: 4292; SEQ ID NO: 4293; SEQ ID NO: 4294; SEQ ID NO: 4295; SEQ ID NO: 4296; SEQ ID NO: 4297; SEQ ID NO: 4298; SEQ ID NO: 4299; SEQ ID NO: 4300; SEQ ID NO: 4301; SEQ ID NO: 4302; SEQ ID NO: 4303; SEQ ID NO: 4304; SEQ ID NO: 4305; SEQ ID NO: 4306; SEQ ID NO: 4307; SEQ ID NO: 4308; SEQ ID NO: 4309; SEQ ID NO: 4310; SEQ ID NO: 4311; SEQ ID NO: 4312; SEQ ID NO: 4313; SEQ ID NO: 4314; SEQ ID NO: 4315; SEQ ID NO: 4316; SEQ ID NO: 4317; SEQ ID NO: 4318; SEQ ID NO: 4319; SEQ ID NO: 4320; SEQ ID NO: 4321; SEQ ID NO: 4322; SEQ ID NO: 4323; SEQ ID NO: 4324; SEQ ID NO: 4325; SEQ ID NO: 4326; SEQ ID NO: 4327; SEQ ID NO: 4328; SEQ ID NO: 4329; SEQ ID NO: 4330; SEQ ID NO: 4331; SEQ ID NO: 4332; SEQ ID NO: 4333; SEQ ID NO: 4334; SEQ ID NO: 4335; SEQ ID NO: 4336; SEQ ID NO: 4337; SEQ ID NO: 4338; SEQ ID NO: 4339; SEQ ID NO: 4340; SEQ ID NO: 4341; SEQ ID NO: 4342; SEQ ID NO: 4343; SEQ ID NO: 4344; SEQ ID NO: 4345; SEQ ID NO: 4346; SEQ ID NO: 4347; SEQ ID NO: 4348; SEQ ID NO: 4349; SEQ ID NO: 4350; SEQ ID NO: 4351; SEQ ID NO: 4352; SEQ ID NO: 4353; SEQ ID NO: 4354; SEQ ID NO: 4355; SEQ ID NO: 4356; SEQ ID NO: 4357; SEQ ID NO: 4358; SEQ ID NO: 4359; SEQ ID NO: 4360; SEQ ID NO: 4361; SEQ ID NO: 4362; SEQ ID NO: 4363; SEQ ID NO: 4364; SEQ ID NO: 4365; SEQ ID NO: 4366; SEQ ID NO: 4367; SEQ ID NO: 4368; SEQ ID NO: 4369; SEQ ID NO: 4370; SEQ ID NO: 4371; SEQ ID NO: 4372; SEQ ID NO: 4373; SEQ ID NO: 4374; SEQ ID NO: 4375; SEQ ID NO: 4376; SEQ ID NO: 4377; SEQ ID NO: 4378; SEQ ID NO: 4379; SEQ ID NO: 4380; SEQ ID NO: 4381; SEQ ID NO: 4382; SEQ ID NO: 4383; SEQ ID NO: 4384; SEQ ID NO: 4385; SEQ ID NO: 4386; SEQ ID NO: 4387; SEQ ID NO: 4388; and SEQ ID NO: 9622; SEQ ID NO: 9623; SEQ ID NO: 9624 and SEQ ID NO: 9625, or a complement therof.

In one embodiment, the H. pylori secreted polypeptide or a fragment thereof is an H. pylori periplasmic polypeptide or a fragment thereof encoded by a nucleic acid having a nucleotide sequence selected from the group consisting of SEQ ID NO: 4016; SEQ ID NO: 4017 and SEQ ID NO: 4018, or a complement thereof.

In another embodiment, the H. pylori secreted polypeptide or a fragment thereof is an H. pylori polypeptide or a fragment thereof involved in secretion and adhesion encoded by a nucleic acid having a nucleotide sequence selected from the group consisting of SEQ ID NO: 4019; SEQ ID NO: 4020; SEQ ID NO: 4021; SEQ ID NO: 4022; SEQ ID NO: 4023; SEQ ID NO: 4024; SEQ ID NO: 4025; SEQ ID NO: 4026 and SEQ ID NO: 9622, or a complement thereof.

In another embodiment, the H. pylori secreted polypeptide or a fragment thereof is an H. pylori chaperone polypeptide or a fragment thereof encoded by a nucleic acid having a nucleotide sequence selected from the group consisting of SEQ ID NO: 4027; SEQ ID NO: 4028; SEQ ID NO: 4029 and SEQ ID NO: 4030, or a complement thereof.

Particularly preferred is an isolated nucleic acid having a nucleotide sequence encoding an H. pylori cellular polypeptide or a fragment thereof, the nucleic acid selected from the group consisting of SEQ ID NO: 4389; SEQ ID NO: 4390; SEQ ID NO: 4391; SEQ ID NO: 4392; SEQ ID NO: 4393; SEQ ID NO: 4394; SEQ ID NO: 4395; SEQ ID NO: 4396; SEQ ID NO: 4397; SEQ ID NO: 4398; SEQ ID NO: 4399; SEQ ID NO: 4400; SEQ ID NO: 4401; SEQ ID NO: 4402; SEQ ID NO: 4403; SEQ ID NO: 4404; SEQ ID NO: 4405; SEQ ID NO: 4406; SEQ ID NO: 4407; SEQ ID NO: 4408; SEQ ID NO: 4409; SEQ ID NO: 4410; SEQ ID NO: 4411; SEQ ID NO: 4412; SEQ ID NO: 4413; SEQ ID NO: 4414; SEQ ID NO: 4415; SEQ ID NO: 4416; SEQ ID NO: 4417; SEQ ID NO: 4418; SEQ ID NO: 4419; SEQ ID NO: 4420; SEQ ID NO: 4421; SEQ ID NO: 4422; SEQ ID NO: 4423; SEQ ID NO: 4424; SEQ ID NO: 4425; SEQ ID NO: 4426; SEQ ID NO: 4427; SEQ ID NO: 4428; SEQ ID NO: 4429; SEQ ID NO: 4430; SEQ ID NO: 4431; SEQ ID NO: 4432; SEQ ID NO: 4433; SEQ ID NO: 4434; SEQ ID NO: 4435; SEQ ID NO: 4436; SEQ ID NO: 4437; SEQ ID NO: 4438; SEQ ID NO: 4439; SEQ ID NO: 4440; SEQ ID NO: 4441; SEQ ID NO: 4442; SEQ ID NO: 4443; SEQ ID NO: 4444; SEQ ID NO: 4445; SEQ ID NO: 4446; SEQ ID NO: 4447; SEQ ID NO: 4448; SEQ ID NO: 4449; SEQ ID NO: 4450; SEQ ID NO: 4451; SEQ ID NO: 4452; SEQ ID NO: 4453; SEQ ID NO: 4454; SEQ ID NO: 4455; SEQ ID NO: 4456; SEQ ID NO: 4457; SEQ ID NO: 4458; SEQ ID NO: 4459; SEQ ID NO: 4460; SEQ ID NO: 4461; SEQ ID NO: 4462; SEQ ID NO: 4463; SEQ ID NO: 4464; SEQ ID NO: 4465; SEQ ID NO: 4466; SEQ ID NO: 4467; SEQ ID NO: 4468; SEQ ID NO: 4469; SEQ ID NO: 4470; SEQ ID NO: 4471; SEQ ID NO: 4472; SEQ ID NO: 4473; SEQ ID NO: 4474; SEQ ID NO: 4475; SEQ ID NO: 4476; SEQ ID NO: 4477; SEQ ID NO: 4478; SEQ ID NO: 4479; SEQ ID NO: 4480; SEQ ID NO: 4481; SEQ ID NO: 4482; SEQ ID NO: 4483; SEQ ID NO: 4484; SEQ ID NO: 4485; SEQ ID NO: 4486; SEQ ID NO: 4487; SEQ ID NO: 4488; SEQ ID NO: 4489; SEQ ID NO: 4490; SEQ ID NO: 4491; SEQ ID NO: 4492; SEQ ID NO: 4493; SEQ ID NO: 4494; SEQ ID NO: 4495; SEQ ID NO: 4496; SEQ ID NO: 4497; SEQ ID NO: 4498; SEQ ID NO: 4499; SEQ ID NO: 4500; SEQ ID NO: 4501; SEQ ID NO: 4502; SEQ ID NO: 4503; SEQ ID NO: 4504; SEQ ID NO: 4505; SEQ ID NO: 4506; SEQ ID NO: 4507; SEQ ID NO: 4508; SEQ ID NO: 4509; SEQ ID NO: 4510; SEQ ID NO: 4511; SEQ ID NO: 4512; SEQ ID NO: 4513; SEQ ID NO: 4514; SEQ ID NO: 4515; SEQ ID NO: 4516; SEQ ID NO: 4517; SEQ ID NO: 4518; SEQ ID NO: 4519; SEQ ID NO: 4520; SEQ ID NO: 4521; SEQ ID NO: 4522; SEQ ID NO: 4523; SEQ ID NO: 4524; SEQ ID NO: 4525; SEQ ID NO: 4526; SEQ ID NO: 4527; SEQ ID NO: 4528; SEQ ID NO: 4529; SEQ ID NO: 4530; SEQ ID NO: 4531; SEQ ID NO: 4532; SEQ ID NO: 4533; SEQ ID NO: 4534; SEQ ID NO: 4535; SEQ ID NO: 4536; SEQ ID NO: 4537; SEQ ID NO: 4538; SEQ ID NO: 4539; SEQ ID NO: 4540; SEQ ID NO: 4541; SEQ ID NO: 4542; SEQ ID NO: 4543; SEQ ID NO: 4544; SEQ ID NO: 4545; SEQ ID NO: 4546; SEQ ID NO: 4547; SEQ ID NO: 4548; SEQ ID NO: 4549; SEQ ID NO: 4550; SEQ ID NO: 4551; SEQ ID NO: 4552; SEQ ID NO: 4553; SEQ ID NO: 4554; SEQ ID NO: 4555; SEQ ID NO: 4556; SEQ ID NO: 4557; SEQ ID NO: 4558; SEQ ID NO: 4559; SEQ ID NO: 4560; SEQ ID NO: 4561; SEQ ID NO: 4562; SEQ ID NO: 4563; SEQ ID NO: 4564; SEQ ID NO: 4565; SEQ ID NO: 4566; SEQ ID NO: 4567; SEQ ID NO: 4568; SEQ ID NO: 4569; SEQ ID NO: 4570; SEQ ID NO: 4571; SEQ ID NO: 4572; SEQ ID NO: 4573; SEQ ID NO: 4574; SEQ ID NO: 4575; SEQ ID NO: 4576; SEQ ID NO: 4577; SEQ ID NO: 4578; SEQ ID NO: 4579; SEQ ID NO: 4580; SEQ ID NO: 4581; SEQ ID NO: 4582; SEQ ID NO: 4583; SEQ ID NO: 4584; SEQ ID NO: 4585; SEQ ID NO: 4586; SEQ ID NO: 4587; SEQ ID NO: 4588; SEQ ID NO: 4589; SEQ ID NO: 4590; SEQ ID NO: 4591; SEQ ID NO: 4592; SEQ ID NO: 4593; SEQ ID NO: 4594; SEQ ID NO: 4595; SEQ ID NO: 4596; SEQ ID NO: 4597; SEQ ID NO: 4598; SEQ ID NO: 4599; SEQ ID NO: 4600; SEQ ID NO: 4601; SEQ ID NO: 4602; SEQ ID NO: 4603; SEQ ID NO: 4604; SEQ ID NO: 4605; SEQ ID NO: 4606; SEQ ID NO: 4607; SEQ ID NO: 4608; SEQ ID NO: 4609; SEQ ID NO: 4610; SEQ ID NO: 4611; SEQ ID NO: 4612; SEQ ID NO: 4613; SEQ ID NO: 4614; SEQ ID NO: 4615; SEQ ID NO: 4616; SEQ ID NO: 4617; SEQ ID NO: 4618; SEQ ID NO: 4619; SEQ ID NO: 4620; SEQ ID NO: 4621; SEQ ID NO: 4622; SEQ ID NO: 4623; SEQ ID NO: 4624; SEQ ID NO: 4625; SEQ ID NO: 4626; SEQ ID NO: 4627; SEQ ID NO: 4628; SEQ ID NO: 4629; SEQ ID NO: 4630; SEQ ID NO: 4631; SEQ ID NO: 4632; SEQ ID NO: 4633; SEQ ID NO: 4634; SEQ ID NO: 4635; SEQ ID NO: 4636; SEQ ID NO: 4637; SEQ ID NO: 4638; SEQ ID NO: 4639; SEQ ID NO: 4640; SEQ ID NO: 4641; SEQ ID NO: 4642; SEQ ID NO: 4643; SEQ ID NO: 4644; SEQ ID NO: 4645; SEQ ID NO: 4646; SEQ ID NO: 4647; SEQ ID NO: 4648; SEQ ID NO: 4649; SEQ ID NO: 4650; SEQ ID NO: 4651; SEQ ID NO: 4652; SEQ ID NO: 4653; SEQ ID NO: 4654; SEQ ID NO: 4655; SEQ ID NO: 4656; SEQ ID NO: 4657; SEQ ID NO: 4658; SEQ ID NO: 4659; SEQ ID NO: 4660; SEQ ID NO: 4661; SEQ ID NO: 4662; SEQ ID NO: 4663; SEQ ID NO: 4664; SEQ ID NO: 4665; SEQ ID NO: 4666; SEQ ID NO: 4667; SEQ ID NO: 4668; SEQ ID NO: 4669; SEQ ID NO: 4670; SEQ ID NO: 4671; SEQ ID NO: 4672; SEQ ID NO: 4673; SEQ ID NO: 4674; SEQ ID NO: 4675; SEQ ID NO: 4676; SEQ ID NO: 4677; SEQ ID NO: 4678; SEQ ID NO: 4679; SEQ ID NO: 4680; SEQ ID NO: 4681; SEQ ID NO: 4682; SEQ ID NO: 4683; SEQ ID NO: 4684; SEQ ID NO: 4685; SEQ ID NO: 4686; SEQ ID NO: 4687; SEQ ID NO: 4688; SEQ ID NO: 4689; SEQ ID NO: 4690; SEQ ID NO: 4691; SEQ ID NO: 4692; SEQ ID NO: 4693; SEQ ID NO: 4694; SEQ ID NO: 4695; SEQ ID NO: 4696; SEQ ID NO: 4697; SEQ ID NO: 4698; SEQ ID NO: 4699; SEQ ID NO: 4700; SEQ ID NO: 4701; SEQ ID NO: 4702; SEQ ID NO: 4703; SEQ ID NO: 4704; SEQ ID NO: 4705; SEQ ID NO: 9626; SEQ ID NO: 9627; SEQ ID NO: 9628; SEQ ID NO: 9629; SEQ ID NO: 9630; SEQ ID NO: 9631; SEQ ID NO: 9632; SEQ ID NO: 9633; SEQ ID NO: 9634; SEQ ID NO: 9635 and SEQ ID NO: 9636, or a complement thereof.

Particularly preferred is an isolated nucleic acid having a nucleotide sequence encoding an H. pylori membrane associated polypeptide or a fragment thereof, the nucleic acid selected from the group consisting of SEQ ID NO: 4706; SEQ ID NO: 4707; SEQ ID NO: 4708; SEQ ID NO: 4709; SEQ ID NO: 4710; SEQ ID NO: 4711; SEQ ID NO: 4712; SEQ ID NO: 4713; SEQ ID NO: 4714; SEQ ID NO: 4715; SEQ ID NO: 4716; SEQ ID NO: 4717; SEQ ID NO: 4718; SEQ ID NO: 4719; SEQ ID NO: 4720; SEQ ID NO: 4721; SEQ ID NO: 4722; SEQ ID NO: 4723; SEQ ID NO: 4724; SEQ ID NO: 4725; SEQ ID NO: 4726; SEQ ID NO: 4727; SEQ ID NO: 4728; SEQ ID NO: 4729; SEQ ID NO: 4730; SEQ ID NO: 4731; SEQ ID NO: 4732; SEQ ID NO: 4733; SEQ ID NO: 4734; SEQ ID NO: 4735; SEQ ID NO: 4736; SEQ ID NO: 4737; SEQ ID NO: 4738; SEQ ID NO: 4739; SEQ ID NO: 4740; SEQ ID NO: 4741; SEQ ID NO: 4742; SEQ ID NO: 4743; SEQ ID NO: 4744; SEQ ID NO: 4745; SEQ ID NO: 4746; SEQ ID NO: 4747; SEQ ID NO: 4748; SEQ ID NO: 4749; SEQ ID NO: 4750; SEQ ID NO: 4751; SEQ ID NO: 4752; SEQ ID NO: 4753; SEQ ID NO: 4754; SEQ ID NO: 4755; SEQ ID NO: 4756; SEQ ID NO: 4757; SEQ ID NO: 4758; SEQ ID NO: 4759; SEQ ID NO: 4760; SEQ ID NO: 4761 and SEQ ID NO: 4762, or a complement thereof.

In another aspect, the invention features a probe having a nucleotide sequence consisting of at least 8 nucleotides of a nucleotide sequence selected from the group consisting of SEQ ID NO: 1-SEQ ID NO: 4762 and SEQ ID NO: 9525-SEQ ID NO: 9636, or a complement thereof.

In another aspect, the invention features an isolated H. pylori polypeptide having an amino acid sequence at least about 60% homologous to an H. pylori polypeptide selected from the group consisting of SEQ ID NO: 4763-SEQ ID NO: 9524 and SEQ ID NO: 9637-SEQ ID NO: 9748.

In another aspect, the invention features an isolated H. pylori polypeptide which is encoded by a nucleic acid having a nucleotide sequence at least about 60% homologous to a nucleotide sequence selected from the group consisting of SEQ ID NO: 1-SEQ ID NO:4762 and SEQ ID NO: 9525-SEQ ID NO: 9636. In one embodiment, the isolated H. pylori polypeptide is encoded by a nucleotide sequence selected from the group consisting of SEQ ID NO: 1-SEQ ID NO: 4762 and SEQ ID NO: 9525-SEQ ID NO: 9636.

In another aspect, the invention features an isolated H. pylori polypeptide which is encoded by a nucleic acid which hybridizes under stringent hybridization conditions to a nucleic acid selected from the group consisting of SEQ ID NO: 1-SEQ ID NO: 4762 and SEQ ID NO: 9525-SEQ ID NO: 9636, or a complement thereof.

In another aspect, the invention features an isolated H. pylori polypeptide having an amino acid sequence selected from the group consisting of SEQ ID NO: 4763-SEQ ID NO: 9524 and SEQ ID NO: 9637-SEQ ID NO: 9748.

Particularly preferred is an isolated H. pylori cell envelope polypeptide or a fragment thereof, wherein the polypeptide has an amino acid sequence selected from the group consisting of SEQ ID NO: 4763; SEQ ID NO: 4764; SEQ ID NO: 4765; SEQ ID NO: 4766; SEQ ID NO: 4767; SEQ ID NO: 4768; SEQ ID NO: 4769; SEQ ID NO: 4770; SEQ ID NO: 4771; SEQ ID NO: 4772; SEQ ID NO: 4773; SEQ ID NO: 4774; SEQ ID NO: 4775; SEQ ID NO: 4776; SEQ ID NO: 4777; SEQ ID NO: 4778; SEQ ID NO: 4779; SEQ ID NO: 4780; SEQ ID NO: 4781; SEQ ID NO: 4782; SEQ ID NO: 4783; SEQ ID NO: 4784; SEQ ID NO: 4785; SEQ ID NO: 4786; SEQ ID NO: 4787; SEQ ID NO: 4788; SEQ ID NO: 4789; SEQ ID NO: 4790; SEQ ID NO: 4791; SEQ ID NO: 4792; SEQ ID NO: 4793; SEQ ID NO: 4794; SEQ ID NO: 4795; SEQ ID NO: 4796; SEQ ID NO: 4797; SEQ ID NO: 4798; SEQ ID NO: 4799; SEQ ID NO: 4800; SEQ ID NO: 4801; SEQ ID NO: 4802; SEQ ID NO: 4803; SEQ ID NO: 4804; SEQ ID NO: 4805; SEQ ID NO: 4806; SEQ ID NO: 4807; SEQ ID NO: 4808; SEQ ID NO: 4809; SEQ ID NO: 4810; SEQ ID NO: 4811; SEQ ID NO: 4812; SEQ ID NO: 4813; SEQ ID NO: 4814; SEQ ID NO: 4815; SEQ ID NO: 4816; SEQ ID NO: 4817; SEQ ID NO: 4818; SEQ ID NO: 4819; SEQ ID NO: 4820; SEQ ID NO: 4821; SEQ ID NO: 4822; SEQ ID NO: 4823; SEQ ID NO: 4824; SEQ ID NO: 4825; SEQ ID NO: 4826; SEQ ID NO: 4827; SEQ ID NO: 4828; SEQ ID NO: 4829; SEQ ID NO: 4830; SEQ ID NO: 4831; SEQ ID NO: 4832; SEQ ID NO: 4833; SEQ ID NO: 4834; SEQ ID NO: 4835; SEQ ID NO: 4836; SEQ ID NO: 4837; SEQ ID NO: 4838; SEQ ID NO: 4839; SEQ ID NO: 4840; SEQ ID NO: 4841; SEQ ID NO: 4842; SEQ ID NO: 4843; SEQ ID NO: 4844; SEQ ID NO: 4845; SEQ ID NO: 4846; SEQ ID NO: 4847; SEQ ID NO: 4848; SEQ ID NO: 4849; SEQ ID NO: 4850; SEQ ID NO: 4851; SEQ ID NO: 4852; SEQ ID NO: 4853; SEQ ID NO: 4854; SEQ ID NO: 4855; SEQ ID NO: 4856; SEQ ID NO: 4857; SEQ ID NO: 4858; SEQ ID NO: 4859; SEQ ID NO: 4860; SEQ ID NO: 4861; SEQ ID NO: 4862; SEQ ID NO: 4863; SEQ ID NO: 4864; SEQ ID NO: 4865; SEQ ID NO: 4866; SEQ ID NO: 4867; SEQ ID NO: 4868; SEQ ID NO: 4869; SEQ ID NO: 4870; SEQ ID NO: 4871; SEQ ID NO: 4872; SEQ ID NO: 4873; SEQ ID NO: 4874; SEQ ID NO: 4875; SEQ ID NO: 4876; SEQ ID NO: 4877; SEQ ID NO: 4878; SEQ ID NO: 4879; SEQ ID NO: 4880; SEQ ID NO: 4881; SEQ ID NO: 4882; SEQ ID NO: 4883; SEQ ID NO: 4884; SEQ ID NO: 4885; SEQ ID NO: 4886; SEQ ID NO: 4887; SEQ ID NO: 4888; SEQ ID NO: 4889; SEQ ID NO: 4890; SEQ ID NO: 4891; SEQ ID NO: 4892; SEQ ID NO: 4893; SEQ ID NO: 4894; SEQ ID NO: 4895; SEQ ID NO: 4896; SEQ ID NO: 4897; SEQ ID NO: 4898; SEQ ID NO: 4899; SEQ ID NO: 4900; SEQ ID NO: 4901; SEQ ID NO: 4902; SEQ ID NO: 4903; SEQ ID NO: 4904; SEQ ID NO: 4905; SEQ ID NO: 4906; SEQ ID NO: 4907; SEQ ID NO: 4908; SEQ ID NO: 4909; SEQ ID NO: 4910; SEQ ID NO: 4911; SEQ ID NO: 4912; SEQ ID NO: 4913; SEQ ID NO: 4914; SEQ ID NO: 4915; SEQ ID NO: 4916; SEQ ID NO: 4917; SEQ ID NO: 4918; SEQ ID NO: 4919; SEQ ID NO: 4920; SEQ ID NO: 4921; SEQ ID NO: 4922; SEQ ID NO: 4923; SEQ ID NO: 4924; SEQ ID NO: 4925; SEQ ID NO: 4926; SEQ ID NO: 4927; SEQ ID NO: 4928; SEQ ID NO: 4929; SEQ ID NO: 4930; SEQ ID NO: 4931; SEQ ID NO: 4932; SEQ ID NO: 4933; SEQ ID NO: 4934; SEQ ID NO: 4935; SEQ ID NO: 4936; SEQ ID NO: 4937; SEQ ID NO: 4938; SEQ ID NO: 4939; SEQ ID NO: 4940; SEQ ID NO: 4941; SEQ ID NO: 4942; SEQ ID NO: 4943; SEQ ID NO: 4944; SEQ ID NO: 4945; SEQ ID NO: 4946; SEQ ID NO: 4947; SEQ ID NO: 4948; SEQ ID NO: 4949; SEQ ID NO: 4950; SEQ ID NO: 4951; SEQ ID NO: 4952; SEQ ID NO: 4953; SEQ ID NO: 4954; SEQ ID NO: 4955; SEQ ID NO: 4956; SEQ ID NO: 4957; SEQ ID NO: 4958; SEQ ID NO: 4959; SEQ ID NO: 4960; SEQ ID NO: 4961; SEQ ID NO: 4962; SEQ ID NO: 4963; SEQ ID NO: 4964; SEQ ID NO: 4965; SEQ ID NO: 4966; SEQ ID NO: 4967; SEQ ID NO: 4968; SEQ ID NO: 4969; SEQ ID NO: 4970; SEQ ID NO: 4971; SEQ ID NO: 4972; SEQ ID NO: 4973; SEQ ID NO: 4974; SEQ ID NO: 4975; SEQ ID NO: 4976; SEQ ID NO: 4977; SEQ ID NO: 4978; SEQ ID NO: 4979; SEQ ID NO: 4980; SEQ ID NO: 4981; SEQ ID NO: 4982; SEQ ID NO: 4983; SEQ ID NO: 4984; SEQ ID NO: 4985; SEQ ID NO: 4986; SEQ ID NO: 4987; SEQ ID NO: 4988; SEQ ID NO: 4989; SEQ ID NO: 4990; SEQ ID NO: 4991; SEQ ID NO: 4992; SEQ ID NO: 4993; SEQ ID NO: 4994; SEQ ID NO: 4995; SEQ ID NO: 4996; SEQ ID NO: 4997; SEQ ID NO: 4998; SEQ ID NO: 4999; SEQ ID NO: 5000; SEQ ID NO: 5001; SEQ ID NO: 5002; SEQ ID NO: 5003; SEQ ID NO: 5004; SEQ ID NO: 5005; SEQ ID NO: 5006; SEQ ID NO: 5007; SEQ ID NO: 5008; SEQ ID NO: 5009; SEQ ID NO: 5010; SEQ ID NO: 5011; SEQ ID NO: 5012; SEQ ID NO: 5013; SEQ ID NO: 5014; SEQ ID NO: 5015; SEQ ID NO: 5016; SEQ ID NO: 5017; SEQ ID NO: 5018; SEQ ID NO: 5019; SEQ ID NO: 5020; SEQ ID NO: 5021; SEQ ID NO: 5022; SEQ ID NO: 5023; SEQ ID NO: 5024; SEQ ID NO: 5025; SEQ ID NO: 5026; SEQ ID NO: 5027; SEQ ID NO: 5028; SEQ ID NO: 5029; SEQ ID NO: 5030; SEQ ID NO: 5031; SEQ ID NO: 5032; SEQ ID NO: 5033; SEQ ID NO: 5034; SEQ ID NO: 5035; SEQ ID NO: 5036; SEQ ID NO: 5037; SEQ ID NO: 5038; SEQ ID NO: 5039; SEQ ID NO: 5040; SEQ ID NO: 5041; SEQ ID NO: 5042; SEQ ID NO: 5043; SEQ ID NO: 5044; SEQ ID NO: 5045; SEQ ID NO: 5046; SEQ ID NO: 5047; SEQ ID NO: 5048; SEQ ID NO: 5049; SEQ ID NO: 5050; SEQ ID NO: 5051; SEQ ID NO: 5052; SEQ ID NO: 5053; SEQ ID NO: 5054; SEQ ID NO: 5055; SEQ ID NO: 5056; SEQ ID NO: 5057; SEQ ID NO: 5058; SEQ ID NO: 5059; SEQ ID NO: 5060; SEQ ID NO: 5061; SEQ ID NO: 5062; SEQ ID NO: 5063; SEQ ID NO: 5064; SEQ ID NO: 5065; SEQ ID NO: 5066; SEQ ID NO: 5067; SEQ ID NO: 5068; SEQ ID NO: 5069; SEQ ID NO: 5070; SEQ ID NO: 5071; SEQ ID NO: 5072; SEQ ID NO: 5073; SEQ ID NO: 5074; SEQ ID NO: 5075; SEQ ID NO: 5076; SEQ ID NO: 5077; SEQ ID NO: 5078; SEQ ID NO: 5079; SEQ ID NO: 5080; SEQ ID NO: 5081; SEQ ID NO: 5082; SEQ ID NO: 5083; SEQ ID NO: 5084; SEQ ID NO: 5085; SEQ ID NO: 5086; SEQ ID NO: 5087; SEQ ID NO: 5088; SEQ ID NO: 5089; SEQ ID NO: 5090; SEQ ID NO: 5091; SEQ ID NO: 5092; SEQ ID NO: 5093; SEQ ID NO: 5094; SEQ ID NO: 5095; SEQ ID NO: 5096; SEQ ID NO: 5097; SEQ ID NO: 5098; SEQ ID NO: 5099; SEQ ID NO: 5100; SEQ ID NO: 5101; SEQ ID NO: 5102, SEQ ID NO: 5103; SEQ ID NO: 5104; SEQ ID NO: 5105; SEQ ID NO: 5106; SEQ ID NO: 5107; SEQ ID NO: 5108; SEQ ID NO:5109; SEQ ID NO: 5110; SEQ ID NO:5111; SEQ ID NO:5112; SEQ ID NO: 5113; SEQ ID NO: 5114; SEQ ID NO: 5115; SEQ ID NO: 5116; SEQ ID NO: 5117; SEQ ID NO: 5118; SEQ ID NO: 5119; SEQ ID NO: 5120; SEQ ID NO: 5121; SEQ ID NO: 5122; SEQ ID NO: 5123; SEQ ID NO: 5124; SEQ ID NO: 5125; SEQ ID NO: 5126; SEQ ID NO: 5127; SEQ ID NO: 5128; SEQ ID NO: 5129; SEQ ID NO: 5130; SEQ ID NO: 5131; SEQ ID NO: 5132; SEQ ID NO: 5133; SEQ ID NO: 5134; SEQ ID NO: 5135; SEQ ID NO: 5136; SEQ ID NO: 5137; SEQ ID NO: 5138; SEQ ID NO: 5139; SEQ ID NO: 5140; SEQ ID NO: 5141; SEQ ID NO: 5142; SEQ ID NO: 5143; SEQ ID NO: 5144; SEQ ID NO: 5145; SEQ ID NO: 5146; SEQ ID NO: 5147; SEQ ID NO: 5148; SEQ ID NO: 5149; SEQ ID NO: 5150; SEQ ID NO: 5151; SEQ ID NO: 5152; SEQ ID NO: 5153; SEQ ID NO: 5154; SEQ ID NO: 5155; SEQ ID NO: 5156; SEQ ID NO: 5157; SEQ ID NO: 5158; SEQ ID NO: 5159; SEQ ID NO: 5160; SEQ ID NO: 5161; SEQ ID NO: 5162; SEQ ID NO: 5163; SEQ ID NO: 5164; SEQ ID NO: 5165; SEQ ID NO: 5166; SEQ ID NO: 5167; SEQ ID NO: 5168; SEQ ID NO: 5169; SEQ ID NO: 5170; SEQ ID NO: 5171; SEQ ID NO: 5172; SEQ ID NO: 5173; SEQ ID NO: 5174; SEQ ID NO: 5175; SEQ ID NO: 5176; SEQ ID NO: 5177; SEQ ID NO: 5178; SEQ ID NO: 5179; SEQ ID NO: 5180; SEQ ID NO: 5181; SEQ ID NO: 5182; SEQ ID NO: 5183; SEQ ID NO: 5184; SEQ ID NO: 5185; SEQ ID NO: 5186; SEQ ID NO: 5187; SEQ ID NO: 5188; SEQ ID NO: 5189; SEQ ID NO: 5190; SEQ ID NO: 5191; SEQ ID NO: 5192; SEQ ID NO: 5193; SEQ ID NO: 5194; SEQ ID NO: 5195; SEQ ID NO: 5196; SEQ ID NO: 5197; SEQ ID NO: 5198; SEQ ID NO: 5199; SEQ ID NO: 5200; SEQ ID NO: 5201; SEQ ID NO: 5202; SEQ ID NO: 5203; SEQ ID NO: 5204; SEQ ID NO: 5205; SEQ ID NO: 5206; SEQ ID NO: 5207; SEQ ID NO: 5208; SEQ ID NO: 5209; SEQ ID NO: 5210; SEQ ID NO: 5211; SEQ ID NO: 5212; SEQ ID NO: 5213; SEQ ID NO: 5214; SEQ ID NO: 5215; SEQ ID NO: 5216; SEQ ID NO: 5217; SEQ ID NO: 5218; SEQ ID NO: 5219; SEQ ID NO: 5220; SEQ ID NO: 5221; SEQ ID NO: 5222; SEQ ID NO: 5223; SEQ ID NO: 5224; SEQ ID NO: 5225; SEQ ID NO: 5226; SEQ ID NO: 5227; SEQ ID NO: 5228; SEQ ID NO: 5229; SEQ ID NO: 5230; SEQ ID NO: 5231; SEQ ID NO: 5232; SEQ ID NO: 5233; SEQ ID NO: 5234; SEQ ID NO: 5235; SEQ ID NO: 5236; SEQ ID NO: 5237; SEQ ID NO: 5238; SEQ ID NO: 5239; SEQ ID NO: 5240; SEQ ID NO: 5241; SEQ ID NO: 5242; SEQ ID NO: 5243; SEQ ID NO: 5244; SEQ ID NO: 5245; SEQ ID NO: 5246; SEQ ID NO: 5247; SEQ ID NO: 5248; SEQ ID NO: 5249; SEQ ID NO: 5250; SEQ ID NO: 5251; SEQ ID NO: 5252; SEQ ID NO: 5253; SEQ ID NO: 5254; SEQ ID NO: 5255; SEQ ID NO: 5256; SEQ ID NO: 52.57; SEQ ID NO: 5258; SEQ ID NO: 5259; SEQ ID NO: 5260; SEQ ID NO: 5261; SEQ ID NO: 5262; SEQ ID NO: 5263; SEQ ID NO: 5264; SEQ ID NO: 5265; SEQ ID NO: 5266; SEQ ID NO: 5267; SEQ ID NO: 5268; SEQ ID NO: 5269; SEQ ID NO: 5270; SEQ ID NO: 5271; SEQ ID NO: 5272; SEQ ID NO: 5273; SEQ ID NO: 5274; SEQ ID NO: 5275; SEQ ID NO: 5276; SEQ ID NO: 5277; SEQ ID NO: 5278; SEQ ID NO: 5279; SEQ ID NO: 5280; SEQ ID NO: 5281; SEQ ID NO: 5282; SEQ ID NO: 5283; SEQ ID NO: 5284; SEQ ID NO: 5285; SEQ ID NO: 5286; SEQ ID NO: 5287; SEQ ID NO: 5288; SEQ ID NO: 5289; SEQ ID NO: 5290; SEQ ID NO: 5291; SEQ ID NO: 5292; SEQ ID NO: 5293; SEQ ID NO: 5294; SEQ ID NO: 5295; SEQ ID NO: 5296; SEQ ID NO: 5297; SEQ ID NO: 5298; SEQ ID NO: 5299; SEQ ID NO: 5300; SEQ ID NO: 5301; SEQ ID NO: 5302; SEQ ID NO: 5303; SEQ ID NO: 5304; SEQ ID NO: 5305; SEQ ID NO: 5306; SEQ ID NO: 5307; SEQ ID NO: 5308; SEQ ID NO: 5309; SEQ ID NO: 5310; SEQ ID NO: 5311; SEQ ID NO: 5312; SEQ ID NO: 5313; SEQ ID NO: 5314; SEQ ID NO: 5315; SEQ ID NO: 5316; SEQ ID NO: 5317; SEQ ID NO: 5318; SEQ ID NO: 5319; SEQ ID NO: 5320; SEQ ID NO: 5321; SEQ ID NO: 5322; SEQ ID NO: 5323; SEQ ID NO: 5324; SEQ ID NO: 5325; SEQ ID NO: 5326; SEQ ID NO: 5327; SEQ ID NO: 5328; SEQ ID NO: 5329; SEQ ID NO: 5330; SEQ ID NO: 5331; SEQ ID NO: 5332; SEQ ID NO: 5333; SEQ ID NO: 5334; SEQ ID NO: 5335; SEQ ID NO: 5336; SEQ ID NO: 5337; SEQ ID NO: 5338; SEQ ID NO: 5339; SEQ ID NO: 5340; SEQ ID NO: 5341; SEQ ID NO: 5342; SEQ ID NO: 5343; SEQ ID NO: 5344; SEQ ID NO: 5345; SEQ ID NO: 5346; SEQ ID NO: 5347; SEQ ID NO: 5348; SEQ ID NO: 5349; SEQ ID NO: 5350; SEQ ID NO: 5351; SEQ ID NO: 5352; SEQ ID NO: 5353; SEQ ID NO: 5354; SEQ ID NO: 5355; SEQ ID NO: 5356; SEQ ID NO: 5357; SEQ ID NO: 5358; SEQ ID NO: 5359; SEQ ID NO: 5360; SEQ ID NO: 5361; SEQ ID NO: 5362; SEQ ID NO: 5363; SEQ ID NO: 5364; SEQ ID NO: 5365; SEQ ID NO: 5366; SEQ ID NO: 5367; SEQ ID NO: 5368; SEQ ID NO: 5369; SEQ ID NO: 5370; SEQ ID NO: 5371; SEQ ID NO: 5372; SEQ ID NO: 5373; SEQ ID NO: 5374; SEQ ID NO: 5375; SEQ ID NO: 5376; SEQ ID NO: 5377; SEQ ID NO: 5378; SEQ ID NO: 5379; SEQ ID NO: 5380; SEQ ID NO: 5381; SEQ ID NO: 5382; SEQ ID NO: 5383; SEQ ID NO: 5384; SEQ ID NO: 5385; SEQ ID NO: 5386; SEQ ID NO: 5387; SEQ ID NO: 5388; SEQ ID NO: 5389; SEQ ID NO: 5390; SEQ ID NO: 5391; SEQ ID NO: 5392; SEQ ID NO: 5393; SEQ ID NO: 5394; SEQ ID NO: 5395; SEQ ID NO: 5396; SEQ ID NO: 5397; SEQ ID NO: 5398; SEQ ID NO: 5399; SEQ ID NO: 5400; SEQ ID NO: 5401; SEQ ID NO: 5402; SEQ ID NO: 5403; SEQ ID NO: 5404; SEQ ID NO: 5405; SEQ ID NO: 5406; SEQ ID NO: 5407; SEQ ID NO: 5408; SEQ ID NO: 5409; SEQ ID NO: 5410; SEQ ID NO: 5411; SEQ ID NO: 5412; SEQ ID NO: 5413; SEQ ID NO: 5414; SEQ ID NO: 5415; SEQ ID NO: 5416; SEQ ID NO: 5417; SEQ ID NO: 5418; SEQ ID NO: 5419; SEQ ID NO: 5420; SEQ ID NO: 5421; SEQ ID NO: 5422; SEQ ID NO: 5423; SEQ ID NO: 5424; SEQ ID NO: 5425; SEQ ID NO: 5426; SEQ ID NO: 5427; SEQ ID NO: 5428; SEQ ID NO: 5429; SEQ ID NO: 5430; SEQ ID NO: 5431; SEQ ID NO: 5432; SEQ ID NO: 5433; SEQ ID NO: 5434; SEQ ID NO: 5435; SEQ ID NO: 5436; SEQ ID NO: 5437; SEQ ID NO: 5438; SEQ ID NO: 5439; SEQ ID NO: 5440; SEQ ID NO: 5441; SEQ ID NO: 5442; SEQ ID NO: 5443; SEQ ID NO: 5444; SEQ ID NO: 5445; SEQ ID NO: 5446; SEQ ID NO: 5447; SEQ ID NO: 5448; SEQ ID NO: 5449; SEQ ID NO: 5450; SEQ ID NO: 5451; SEQ ID NO: 5452; SEQ ID NO: 5453; SEQ ID NO: 5454; SEQ ID NO: 5455; SEQ ID NO: 5456; SEQ ID NO: 5457; SEQ ID NO: 5458; SEQ ID NO: 5459; SEQ ID NO: 5460; SEQ ID NO: 5461; SEQ ID NO: 5462; SEQ ID NO: 5463; SEQ ID NO: 5464; SEQ ID NO: 5465; SEQ ID NO: 5466; SEQ ID NO: 5467; SEQ ID NO: 5468; SEQ ID NO: 5469; SEQ ID NO: 5470; SEQ ID NO: 5471; SEQ ID NO: 5472; SEQ ID NO: 5473; SEQ ID NO: 5474; SEQ ID NO: 5475; SEQ ID NO: 5476; SEQ ID NO: 5477; SEQ ID NO: 5478; SEQ ID NO: 5479; SEQ ID NO: 5480; SEQ ID NO: 5481; SEQ ID NO: 5482; SEQ ID NO: 5483; SEQ ID NO: 5484; SEQ ID NO: 5485; SEQ ID NO: 5486; SEQ ID NO: 5487; SEQ ID NO: 5488; SEQ ID NO: 5489; SEQ ID NO: 5490; SEQ ID NO: 5491; SEQ ID NO: 5492; SEQ ID NO: 5493; SEQ ID NO: 5494; SEQ ID NO: 5495; SEQ ID NO: 5496; SEQ ID NO: 5497; SEQ ID NO: 5498; SEQ ID NO: 5499; SEQ ID NO: 5500; SEQ ID NO: 5501; SEQ ID NO: 5502; SEQ ID NO: 5503; SEQ ID NO: 5504; SEQ ID NO: 5505; SEQ ID NO: 5506; SEQ ID NO: 5507; SEQ ID NO: 5508; SEQ ID NO: 5509; SEQ ID NO: 5510; SEQ ID NO: 5511; SEQ ID NO: 5512; SEQ ID NO: 5513; SEQ ID NO: 5514; SEQ ID NO: 5515; SEQ ID NO: 5516; SEQ ID NO: 5517; SEQ ID NO: 5518; SEQ ID NO: 5519; SEQ ID NO: 5520; SEQ ID NO: 5521; SEQ ID NO: 5522; SEQ ID NO: 5523; SEQ ID NO: 5524; SEQ ID NO: 5525; SEQ ID NO: 5526; SEQ ID NO: 5527; SEQ ID NO: 5528; SEQ ID NO: 5529; SEQ ID NO: 5530; SEQ ID NO: 5531; SEQ ID NO: 5532; SEQ ID NO: 5533; SEQ ID NO: 5534; SEQ ID NO: 5535; SEQ ID NO: 5536; SEQ ID NO: 5537; SEQ ID NO: 5538; SEQ ID NO: 5539; SEQ ID NO: 5540; SEQ ID NO: 5541; SEQ ID NO: 5542; SEQ ID NO: 5543; SEQ ID NO: 5544; SEQ ID NO: 5545; SEQ ID NO: 5546; SEQ ID NO: 5547; SEQ ID NO: 5548; SEQ ID NO: 5549; SEQ ID NO: 5550; SEQ ID NO: 5551; SEQ ID NO: 5552; SEQ ID NO: 5553; SEQ ID NO: 5554; SEQ ID NO: 5555; SEQ ID NO: 5556; SEQ ID NO: 5557; SEQ ID NO: 5558; SEQ ID NO: 5559; SEQ ID NO: 5560; SEQ ID NO: 5561; SEQ ID NO: 5562; SEQ ID NO: 5563; SEQ ID NO: 5564; SEQ ID NO: 5565; SEQ ID NO: 5566; SEQ ID NO: 5567; SEQ ID NO: 5568; SEQ ID NO: 5569; SEQ ID NO: 5570; SEQ ID NO: 5571; SEQ ID NO: 5572; SEQ ID NO: 5573; SEQ ID NO: 5574; SEQ ID NO: 5575; SEQ ID NO: 5576; SEQ ID NO: 5577; SEQ ID NO: 5578; SEQ ID NO: 5579; SEQ ID NO: 5580; SEQ ID NO: 5581; SEQ ID NO: 5582; SEQ ID NO: 5583; SEQ ID NO: 5584; SEQ ID NO: 5585; SEQ ID NO: 5586; SEQ ID NO: 5587; SEQ ID NO: 5588; SEQ ID NO: 5589; SEQ ID NO: 5590; SEQ ID NO: 5591; SEQ ID NO: 5592; SEQ ID NO: 5593; SEQ ID NO: 5594; SEQ ID NO: 5595; SEQ ID NO: 5596; SEQ ID NO: 5597; S-Q ID NO: 5598; SEQ ID NO: 5599; SEQ ID NO: 5600; SEQ ID NO: 5601; SEQ ID NO: 5602; SEQ ID NO: 5603; SEQ ID NO: 5604; SEQ ID NO: 5605; SEQ ID NO: 5606; SEQ ID NO: 5607; SEQ ID NO: 5608; SEQ ID NO: 5609; SEQ ID NO: 5610; SEQ ID NO: 5611; SEQ ID NO: 5612; SEQ ID NO: 5613; SEQ ID NO: 5614; SEQ ID NO: 5615; SEQ ID NO: 5616; SEQ ID NO: 5617; SEQ ID NO: 5618; SEQ ID NO: 5619; SEQ ID NO: 5620; SEQ ID NO: 5621; SEQ ID NO: 5622; SEQ ID NO: 5623; SEQ ID NO: 5624; SEQ ID NO: 5625; SEQ ID NO: 5626; SEQ ID NO: 5627; SEQ ID NO: 5628; SEQ ID NO: 5629; SEQ ID NO: 5630; SEQ ID NO: 5631; SEQ ID NO: 5632; SEQ ID NO: 5633; SEQ ID NO: 5634; SEQ ID NO: 5635; SEQ ID NO: 5636; SEQ ID NO: 5637; SEQ ID NO: 5638; SEQ ID NO: 5639; SEQ ID NO: 5640; SEQ ID NO: 5641; SEQ ID NO: 5642; SEQ ID NO: 5643; SEQ ID NO: 5644; SEQ ID NO: 5645; SEQ ID NO: 5646; SEQ ID NO: 5647; SEQ ID NO: 5648; SEQ ID NO: 5649; SEQ ID NO: 5650; SEQ ID NO: 5651; SEQ ID NO: 5652; SEQ ID NO: 5653; SEQ ID NO: 5654; SEQ ID NO: 5655; SEQ ID NO: 5656; SEQ ID NO: 5657; SEQ ID NO: 5658; SEQ ID NO: 5659; SEQ ID NO: 5660; SEQ ID NO: 5661; SEQ ID NO: 5662; SEQ ID NO: 5663; SEQ ID NO: 5664; SEQ ID NO: 5665; SEQ ID NO: 5666; SEQ ID NO: 5667; SEQ ID NO: 5668; SEQ ID NO: 5669; SEQ ID NO: 5670; SEQ ID NO: 5671; SEQ ID NO: 5672; SEQ ID NO: 5673; SEQ ID NO: 5674; SEQ ID NO: 5675; SEQ ID NO: 5676; SEQ ID NO: 5677; SEQ ID NO: 5678; SEQ ID NO: 5679; SEQ ID NO: 5680; SEQ ID NO: 5681; SEQ ID NO: 5682; SEQ ID NO: 5683; SEQ ID NO: 5684; SEQ ID NO: 5685; SEQ ID NO: 5686; SEQ ID NO: 5687; SEQ ID NO: 5688; SEQ ID NO: 5689; SEQ ID NO: 5690; SEQ ID NO: 5691; SEQ ID NO: 5692; SEQ ID NO: 5693; SEQ ID NO: 5694; SEQ ID NO: 5695; SEQ ID NO: 5696; SEQ ID NO: 5697; SEQ ID NO: 5698; SEQ ID NO: 5699; SEQ ID NO: 5700; SEQ ID NO: 5701; SEQ ID NO: 5702; SEQ ID NO: 5703; SEQ ID NO: 5704; SEQ ID NO: 5705; SEQ ID NO: 5706; SEQ ID NO: 5707; SEQ ID NO: 5708; SEQ ID NO: 5709; SEQ ID NO: 5710; SEQ ID NO: 5711; SEQ ID NO: 5712; SEQ ID NO: 5713; SEQ ID NO: 5714; SEQ ID NO: 5715; SEQ ID NO: 5716; SEQ ID NO: 5717; SEQ ID NO: 5718; SEQ ID NO: 5719; SEQ ID NO:5720; SEQ ID NO:5721; SEQ ID NO:5722; SEQ ID NO:5723; SEQ ID NO: 5724; SEQ ID NO: 5725; SEQ ID NO: 5726; SEQ ID NO: 5727; SEQ ID NO: 5728; SEQ ID NO: 5729; SEQ ID NO: 5730; SEQ ID NO: 5731; SEQ ID NO: 5732; SEQ ID NO: 5733; SEQ ID NO: 5734; SEQ ID NO: 5735; SEQ ID NO: 5736; SEQ ID NO: 5737; SEQ ID NO: 5738; SEQ ID NO: 5739; SEQ ID NO: 5740; SEQ ID NO: 5741; SEQ ID NO: 5742; SEQ ID NO: 5743; SEQ ID NO: 5744; SEQ ID NO: 5745; SEQ ID NO: 5746; SEQ ID NO: 5747; SEQ ID NO: 5748; SEQ ID NO: 5749; SEQ ID NO: 5750; SEQ ID NO: 5751; SEQ ID NO: 5752; SEQ ID NO: 5753; SEQ ID NO: 5754; SEQ ID NO: 5755; SEQ ID NO: 5756; SEQ ID NO: 5757; SEQ ID NO: 5758; SEQ ID NO: 5759; SEQ ID NO: 5760; SEQ ID NO: 5761; SEQ ID NO: 5762; SEQ ID NO: 5763; SEQ ID NO: 5764; SEQ ID NO: 5765; SEQ ID NO: 5766; SEQ ID NO: 5767; SEQ ID NO: 5768; SEQ ID NO: 5769; SEQ ID NO: 5770; SEQ ID NO: 5771; SEQ ID NO: 5772; SEQ ID NO: 5773; SEQ ID NO: 5774; SEQ ID NO: 5775; SEQ ID NO: 5776; SEQ ID NO: 5777; SEQ ID NO: 5778; SEQ ID NO: 5779; SEQ ID NO: 5780; SEQ ID NO: 5781; SEQ ID NO: 5782; SEQ ID NO: 5783; SEQ ID NO: 5784; SEQ ID NO: 5785; SEQ ID NO: 5786; SEQ ID NO: 5787; SEQ ID NO: 5788; SEQ ID NO: 5789; SEQ ID NO: 5790; SEQ ID NO: 5791; SEQ ID NO: 5792; SEQ ID NO: 5793; SEQ ID NO: 5794; SEQ ID NO: 5795; SEQ ID NO: 5796; SEQ ID NO: 5797; SEQ ID NO: 5798; SEQ ID NO: 5799; SEQ ID NO: 5800; SEQ ID NO: 5801; SEQ ID NO: 5802; SEQ ID NO: 5803; SEQ ID NO: 5804; SEQ ID NO: 5805; SEQ ID NO: 5806; SEQ ID NO: 5807; SEQ ID NO: 5808; SEQ ID NO: 5809; SEQ ID NO: 5810; SEQ ID NO: 5811; SEQ ID NO: 5812; SEQ ID NO: 5813; SEQ ID NO: 5814; SEQ ID NO: 5815; SEQ ID NO: 5816; SEQ ID NO: 5817; SEQ ID NO: 5818; SEQ ID NO: 5819; SEQ ID NO: 5820; SEQ ID NO: 5821; SEQ ID NO: 5822; SEQ ID NO: 5823; SEQ ID NO: 5824; SEQ ID NO: 5825; SEQ ID NO: 5826; SEQ ID NO: 5827; SEQ ID NO: 5828; SEQ ID NO: 5829; SEQ ID NO: 5830; SEQ ID NO: 5831; SEQ ID NO: 5832; SEQ ID NO: 5833; SEQ ID NO: 5834; SEQ ID NO: 5835; SEQ ID NO: 5836; SEQ ID NO: 5837; SEQ ID NO: 5838; SEQ ID NO: 5839; SEQ ID NO: 5840; SEQ ID NO: 5841; SEQ ID NO: 5842; SEQ ID NO: 5843; SEQ ID NO: 5844; SEQ ID NO: 5845; SEQ ID NO: 5846; SEQ ID NO: 5847; SEQ ID NO: 5848; SEQ ID NO: 5849; SEQ ID NO: 5850; SEQ ID NO: 5851; SEQ ID NO: 5852; SEQ ID NO: 5853; SEQ ID NO: 5854; SEQ ID NO: 5855; SEQ ID NO: 5856; SEQ ID NO: 5857; SEQ ID NO: 5858; SEQ ID NO: 5859; SEQ ID NO: 5860; SEQ ID NO: 5861; SEQ ID NO: 5862; SEQ ID NO: 5863; SEQ ID NO: 5864; SEQ ID NO: 5865; SEQ ID NO: 5866; SEQ ID NO: 5867; SEQ ID NO: 5868; SEQ ID NO: 5869; SEQ ID NO: 5870; SEQ ID NO: 5871; SEQ ID NO: 5872; SEQ ID NO: 5873; SEQ ID NO: 5874; SEQ ID NO: 5875; SEQ ID NO: 5876; SEQ ID NO: 5877; SEQ ID NO: 5878; SEQ ID NO: 5879; SEQ ID NO: 5880; SEQ ID NO: 5881; SEQ ID NO: 5882; SEQ ID NO: 5883; SEQ ID NO: 5884; SEQ ID NO: 5885; SEQ ID NO: 5886; SEQ ID NO: 5887; SEQ ID NO: 5888; SEQ ID NO: 5889; SEQ ID NO: 5890; SEQ ID NO: 5891; SEQ ID NO: 5892; SEQ ID NO: 5893; SEQ ID NO: 5894; SEQ ID NO: 5895; SEQ ID NO: 5896; SEQ ID NO: 5897; SEQ ID NO: 5898; SEQ ID NO: 5899; SEQ ID NO: 5900; SEQ ID NO: 5901; SEQ ID NO: 5902; SEQ ID NO: 5903; SEQ ID NO: 5904; SEQ ID NO: 5905; SEQ ID NO: 5906; SEQ ID NO: 5907; SEQ ID NO: 5908; SEQ ID NO: 5909; SEQ ID NO: 5910; SEQ ID NO: 5911; SEQ ID NO: 5912; SEQ ID NO: 5913; SEQ ID NO: 5914; SEQ ID NO: 5915; SEQ ID NO: 5916; SEQ ID NO: 5917; SEQ ID NO: 5918; SEQ ID NO: 5919; SEQ ID NO: 5920; SEQ ID NO: 5921; SEQ ID NO: 5922; SEQ ID NO: 5923; SEQ ID NO: 5924; SEQ ID NO: 5925; SEQ ID NO: 5926; SEQ ID NO: 5927; SEQ ID NO: 5928; SEQ ID NO: 5929; SEQ ID NO: 5930; SEQ ID NO: 5931; SEQ ID NO: 5932; SEQ ID NO: 5933; SEQ ID NO: 5934; SEQ ID NO: 5935; SEQ ID NO: 5936; SEQ ID NO: 5937; SEQ ID NO: 5938; SEQ ID NO: 5939; SEQ ID NO: 5940; SEQ ID NO: 5941; SEQ ID NO: 5942; SEQ ID NO: 5943; SEQ ID NO: 5944; SEQ ID NO: 5945; SEQ ID NO: 5946; SEQ ID NO: 5947; SEQ ID NO: 5948; SEQ ID NO: 5949; SEQ ID NO: 5950; SEQ ID NO: 5951; SEQ ID NO: 5952; SEQ ID NO: 5953; SEQ ID NO: 5954; SEQ ID NO: 5955; SEQ ID NO: 5956; SEQ ID NO: 5957; SEQ ID NO: 5958; SEQ ID NO: 5959; SEQ ID NO: 5960; SEQ ID NO: 5961; SEQ ID NO: 5962; SEQ ID NO: 5963; SEQ ID NO: 5964; SEQ ID NO: 5965; SEQ ID NO: 5966; SEQ ID NO: 5967; SEQ ID NO: 5968; SEQ ID NO: 5969; SEQ ID NO: 5970; SEQ ID NO: 5971; SEQ ID NO: 5972; SEQ ID NO: 5973; SEQ ID NO: 5974; SEQ ID NO: 5975; SEQ ID NO: 5976; SEQ ID NO: 5977; SEQ ID NO: 5978; SEQ ID NO: 5979; SEQ ID NO: 5980; SEQ ID NO: 5981; SEQ ID NO: 5982; SEQ ID NO: 5983; SEQ ID NO: 5984; SEQ ID NO: 5985; SEQ ID NO: 5986; SEQ ID NO: 5987; SEQ ID NO: 5988; SEQ ID NO: 5989; SEQ ID NO: 5990; SEQ ID NO: 5991; SEQ ID NO: 5992; SEQ ID NO: 5993; SEQ ID NO: 5994; SEQ ID NO: 5995; SEQ ID NO: 5996; SEQ ID NO: 5997; SEQ ID NO: 5998; SEQ ID NO: 5999; SEQ ID NO: 6000; SEQ ID NO: 6001; SEQ ID NO: 6002; SEQ ID NO: 6003; SEQ ID NO: 6004; SEQ ID NO: 6005; SEQ ID NO: 6006; SEQ ID NO: 6007; SEQ ID NO: 6008; SEQ ID NO: 6009; SEQ ID NO: 6010; SEQ ID NO: 6011; SEQ ID NO: 6012; SEQ ID NO: 6013; SEQ ID NO: 6014; SEQ ID NO: 6015; SEQ ID NO: 6016; SEQ ID NO: 6017; SEQ ID NO: 6018; SEQ ID NO: 6019; SEQ ID NO: 6020; SEQ ID NO: 6021; SEQ ID NO: 6022; SEQ ID NO: 6023; SEQ ID NO: 6024; SEQ ID NO: 6025; SEQ ID NO: 6026; SEQ ID NO: 6027; SEQ ID NO: 6028; SEQ ID NO: 6029; SEQ ID NO: 6030; SEQ ID NO: 6031; SEQ ID NO: 6032; SEQ ID NO: 6033; SEQ ID NO: 6034; SEQ ID NO: 6035; SEQ ID NO: 6036; SEQ ID NO: 6037; SEQ ID NO: 6038; SEQ ID NO: 6039; SEQ ID NO: 6040; SEQ ID NO: 6041; SEQ ID NO: 6042; SEQ ID NO: 6043; SEQ ID NO: 6044; SEQ ID NO: 6045; SEQ ID NO: 6046; SEQ ID NO: 6047; SEQ ID NO: 6048; SEQ ID NO: 6049; SEQ ID NO: 6050; SEQ ID NO: 6051; SEQ ID NO: 6052; SEQ ID NO: 6053; SEQ ID NO: 6054; SEQ ID NO: 6055; SEQ ID NO: 6056; SEQ ID NO: 6057; SEQ ID NO: 6058; SEQ ID NO: 6059; SEQ ID NO: 6060; SEQ ID NO: 6061; SEQ ID NO: 6062; SEQ ID NO: 6063; SEQ ID NO: 6064; SEQ ID NO: 6065; SEQ ID NO: 6066; SEQ ID NO: 6067; SEQ ID NO: 6068; SEQ ID NO: 6069; SEQ ID NO: 6070; SEQ ID NO: 6071; SEQ ID NO: 6072; SEQ ID NO: 6073; SEQ ID NO: 6074; SEQ ID NO: 6075; SEQ ID NO: 6076; SEQ ID NO: 6077; SEQ ID NO: 6078; SEQ ID NO: 6079; SEQ ID NO: 6080; SEQ ID NO: 6081; SEQ ID NO: 6082; SEQ ID NO: 6083; SEQ ID NO: 6084; SEQ ID NO: 6085; SEQ ID NO: 6086; SEQ ID NO: 6087; SEQ ID NO: 6088; SEQ ID NO: 6089; SEQ ID NO: 6090; SEQ ID NO: 6091; SEQ ID NO: 6092; SEQ ID NO: 6093; SEQ ID NO: 6094; SEQ ID NO: 6095; SEQ ID NO: 6096; SEQ ID NO: 6097; SEQ ID NO: 6098; SEQ ID NO: 6099; SEQ ID NO: 6100; SEQ ID NO: 6101; SEQ ID NO: 6102; SEQ ID NO: 6103; SEQ ID NO: 6104; SEQ ID NO: 6105; SEQ ID NO: 6106; SEQ ID NO: 6107; SEQ ID NO: 6108; SEQ ID NO: 6109; SEQ ID NO: 6110; SEQ ID NO: 6111; SEQ ID NO: 6112; SEQ ID NO: 6113; SEQ ID NO: 6114; SEQ ID NO: 6115; SEQ ID NO: 6116; SEQ ID NO: 6117; SEQ ID NO: 6118; SEQ ID NO: 6119; SEQ ID NO: 6120; SEQ ID NO: 6121; SEQ ID NO: 6122; SEQ ID NO: 6123; SEQ ID NO: 6124; SEQ ID NO: 6125; SEQ ID NO: 6126; SEQ ID NO: 6127; SEQ ID NO: 6128; SEQ ID NO: 6129; SEQ ID NO: 6130; SEQ ID NO: 6131; SEQ ID NO: 6132; SEQ ID NO: 6133; SEQ ID NO: 6134; SEQ ID NO: 6135; SEQ ID NO: 6136; SEQ ID NO: 6137; SEQ ID NO: 6138; SEQ ID NO: 6139; SEQ ID NO: 6140; SEQ ID NO: 6141; SEQ ID NO: 6142; SEQ ID NO: 6143; SEQ ID NO: 6144; SEQ ID NO: 6145; SEQ ID NO: 6146; SEQ ID NO: 6147; SEQ ID NO: 6148; SEQ ID NO: 6149; SEQ ID NO: 6150; SEQ ID NO: 6151; SEQ ID NO:6152; SEQ ID NO: 6153; SEQ ID NO: 6154; SEQ ID NO: 6155; SEQ ID NO: 6156; SEQ ID NO: 6157; SEQ ID NO: 6158; SEQ ID NO: 6159; SEQ ID NO: 6160; SEQ ID NO: 6161; SEQ ID NO: 6162; SEQ ID NO: 6163; SEQ ID NO: 6164; SEQ ID NO: 6165; SEQ ID NO: 6166; SEQ ID NO: 6167; SEQ ID NO: 6168; SEQ ID NO: 6169; SEQ ID NO: 6170; SEQ ID NO: 6171; SEQ ID NO: 6172; SEQ ID NO: 6173; SEQ ID NO: 6174; SEQ ID NO: 6175; SEQ ID NO: 6176; SEQ ID NO: 6177; SEQ ID NO: 6178; SEQ ID NO: 6179; SEQ ID NO: 6180; SEQ ID NO: 6181; SEQ ID NO: 6182; SEQ ID NO: 6183; SEQ ID NO: 6184; SEQ ID NO: 6185; SEQ ID NO: 6186; SEQ ID NO: 6187; SEQ ID NO: 6188; SEQ ID NO: 6189; SEQ ID NO: 6190; SEQ ID NO: 6191; SEQ ID NO: 6192; SEQ ID NO: 6193; SEQ ID NO: 6194; SEQ ID NO: 6195; SEQ ID NO: 6196; SEQ ID NO: 6197; SEQ ID NO: 6198; SEQ ID NO: 6199; SEQ ID NO: 6200; SEQ ID NO: 6201; SEQ ID NO: 6202; SEQ ID NO: 6203; SEQ ID NO: 6204; SEQ ID NO: 6205; SEQ ID NO: 6206; SEQ ID NO: 6207; SEQ ID NO: 6208; SEQ ID NO: 6209; SEQ ID NO: 6210; SEQ ID NO: 6211; SEQ ID NO: 6212; SEQ ID NO: 6213; SEQ ID NO: 6214; SEQ ID NO: 6215; SEQ ID NO: 6216; SEQ ID NO: 6217; SEQ ID NO: 6218; SEQ ID NO: 6219; SEQ ID NO: 6220; SEQ ID NO: 6221; SEQ ID NO: 6222; SEQ ID NO: 6223; SEQ ID NO: 6224; SEQ ID NO: 6225; SEQ ID NO: 6226; SEQ ID NO: 6227; SEQ ID NO: 6228; SEQ ID NO: 6229; SEQ ID NO: 6230; SEQ ID NO: 6231; SEQ ID NO: 6232; SEQ ID NO: 6233; SEQ ID NO: 6234; SEQ ID NO: 6235; SEQ ID NO: 6236; SEQ ID NO: 6237; SEQ ID NO: 6238; SEQ ID NO: 6239; SEQ ID NO: 6240; SEQ ID NO: 6241; SEQ ID NO: 6242; SEQ ID NO: 6243; SEQ ID NO: 6244; SEQ ID NO: 6245; SEQ ID NO: 6246; SEQ ID NO: 6247; SEQ ID NO: 6248; SEQ ID NO: 6249; SEQ ID NO: 6250; SEQ ID NO: 6251; SEQ ID NO: 6252; SEQ ID NO: 6253; SEQ ID NO: 6254; SEQ ID NO: 6255; SEQ ID NO: 6256; SEQ ID NO: 6257; SEQ ID NO: 6258; SEQ ID NO: 6259; SEQ ID NO: 6260; SEQ ID NO: 6261; SEQ ID NO: 6262; SEQ ID NO: 6263; SEQ ID NO: 6264; SEQ ID NO: 6265; SEQ ID NO: 6266; SEQ ID NO: 6267; SEQ ID NO: 6268; SEQ ID NO: 6269; SEQ ID NO: 6270; SEQ ID NO: 6271; SEQ ID NO: 6272; SEQ ID NO: 6273; SEQ ID NO: 6274; SEQ ID NO: 6275; SEQ ID NO: 6276; SEQ ID NO: 6277; SEQ ID NO: 6278; SEQ ID NO: 6279; SEQ ID NO: 6280; SEQ ID NO: 6281; SEQ ID NO: 6282; SEQ ID NO: 6283; SEQ ID NO: 6284; SEQ ID NO: 6285; SEQ ID NO: 6286; SEQ ID NO: 6287; SEQ ID NO: 6288; SEQ ID NO: 6289; SEQ ID NO: 6290; SEQ ID NO: 6291; SEQ ID NO: 6292; SEQ ID NO: 6293; SEQ ID NO: 6294; SEQ ID NO: 6295; SEQ ID NO: 6296; SEQ ID NO: 6297; SEQ ID NO: 6298; SEQ ID NO: 6299; SEQ ID NO: 6300; SEQ ID NO: 6301; SEQ ID NO: 6302; SEQ ID NO: 6303; SEQ ID NO: 6304; SEQ ID NO: 6305; SEQ ID NO: 6306; SEQ ID NO: 6307; SEQ ID NO: 6308; SEQ ID NO: 6309; SEQ ID NO: 6310; SEQ ID NO: 6311; SEQ ID NO: 6312; SEQ ID NO: 6313; SEQ ID NO: 6314; SEQ ID NO: 6315; SEQ ID NO: 6316; SEQ ID NO: 6317; SEQ ID NO: 6318; SEQ ID NO: 6319; SEQ ID NO: 6320; SEQ ID NO: 6321; SEQ ID NO: 6322; SEQ ID NO: 6323; SEQ ID NO: 6324; SEQ ID NO: 6325; SEQ ID NO: 6326; SEQ ID NO: 6327; SEQ ID NO: 6328; SEQ ID NO: 6329; SEQ ID NO: 6330; SEQ ID NO: 6331; SEQ ID NO: 6332; SEQ ID NO: 6333; SEQ ID NO: 6334; SEQ ID NO: 6335; SEQ ID NO: 6336; SEQ ID NO: 6337; SEQ ID NO: 9637; SEQ ID NO: 9638; SEQ ID NO: 9639; SEQ ID NO: 9640; SEQ ID NO: 9641; SEQ ID NO: 9642; SEQ ID NO: 9643; SEQ ID NO: 9644; SEQ ID NO: 9645; SEQ ID NO: 9646; SEQ ID NO: 9647; SEQ ID NO: 9648; SEQ ID NO: 9649; SEQ ID NO: 9650; SEQ ID NO: 9651; SEQ ID NO: 9652; SEQ ID NO: 9653; SEQ ID NO: 9654; SEQ ID NO: 9655; SEQ ID NO: 9656; SEQ ID NO: 9657; SEQ ID NO: 9658; SEQ ID NO: 9659; SEQ ID NO: 9660; SEQ ID NO: 9661; SEQ ID NO: 9662; SEQ ID NO: 9663; SEQ ID NO: 9664; SEQ ID NO: 9665; SEQ ID NO: 9666; SEQ ID NO: 9667; SEQ ID NO: 9668; SEQ ID NO: 9669; SEQ ID NO: 9670; SEQ ID NO: 9671; SEQ ID NO: 9672; SEQ ID NO: 9673; SEQ ID NO: 9674; SEQ ID NO: 9675; SEQ ID NO: 9676; SEQ ID NO: 9677; SEQ ID NO: 9678; SEQ ID NO: 9679; SEQ ID NO: 9680; SEQ ID NO: 9681; SEQ ID NO: 9682; SEQ ID NO: 9683; SEQ ID NO: 9684; SEQ ID NO: 9685; SEQ ID NO: 9686; SEQ ID NO: 9687; SEQ ID NO: 9688; SEQ ID NO: 9689; SEQ ID NO: 9690; SEQ ID NO: 9691; SEQ ID NO: 9692; SEQ ID NO: 9693; SEQ ID NO: 9694; SEQ ID NO: 9695; SEQ ID NO: 9696; SEQ ID NO: 9697; SEQ ID NO: 9698; SEQ ID NO: 9699; SEQ ID NO: 9700; SEQ ID NO: 9701; SEQ ID NO: 9702; SEQ ID NO: 9703 and SEQ ID NO: 9704.

In one embodiment, the H. pylori cell envelope polypeptide or a fragment thereof is an H. pylori flagella-associated polypeptide or a fragment thereof having an amino acid sequence selected from the group consisting of SEQ ID NO: 4763; SEQ ID NO: 4764; SEQ ID NO: 4765; SEQ ID NO: 4766; SEQ ID NO: 4767; SEQ ID NO: 4768; SEQ ID NO: 4769; SEQ ID NO: 4770; SEQ ID NO: 4771; SEQ ID NO: 4772; SEQ ID NO: 4773; SEQ ID NO: 4774; SEQ ID NO: 4775; SEQ ID NO: 4776; SEQ ID NO: 4777; SEQ ID NO: 4778; SEQ ID NO: 4779; SEQ ID NO: 4780; SEQ ID NO: 4781; SEQ ID NO: 4782; SEQ ID NO: 4783; SEQ ID NO: 4784; SEQ ID NO: 4785; SEQ ID NO: 4786; SEQ ID NO: 4787; SEQ ID NO: 4788; SEQ ID NO: 4789; SEQ ID NO: 4790; SEQ ID NO: 4791; SEQ ID NO: 4792; SEQ ID NO: 4793; SEQ ID NO: 4794; SEQ ID NO: 4795; SEQ ID NO: 4796; SEQ ID NO: 4797; SEQ ID NO: 4798; SEQ ID NO: 4799; SEQ ID NO: 4800; SEQ ID NO: 4801; SEQ ID NO: 4802; SEQ ID NO: 4803; SEQ ID NO: 4804; SEQ ID NO: 4805; SEQ ID NO: 4806; SEQ ID NO: 4807; SEQ ID NO: 4808; SEQ ID NO: 4809; SEQ ID NO: 4810; SEQ ID NO: 4811; SEQ ID NO: 4812; SEQ ID NO: 4813; SEQ ID NO: 4814; SEQ ID NO: 4815; SEQ ID NO: 4816; SEQ ID NO: 4817; SEQ ID NO: 4818; SEQ ID NO: 4819; SEQ ID NO: 4820; SEQ ID NO: 4821; SEQ ID NO: 4822; SEQ ID NO: 4823; SEQ ID NO: 4824; SEQ ID NO: 4825; SEQ ID NO: 4826; SEQ ID NO: 4827; SEQ ID NO: 4828; SEQ ID NO: 4829; SEQ ID NO: 4830; SEQ ID NO: 4831; SEQ ID NO: 4832; SEQ ID NO: 4833; SEQ ID NO: 4834; SEQ ID NO: 4835; SEQ ID NO: 4836; SEQ ID NO: 4837; SEQ ID NO: 4838; SEQ ID NO: 4839; SEQ ID NO: 4840; SEQ ID NO: 4841; SEQ ID NO: 4842; SEQ ID NO: 4843; SEQ ID NO: 4844; SEQ ID NO: 4845; SEQ ID NO: 4846; SEQ ID NO: 4847; SEQ ID NO: 4848; SEQ ID NO: 4849; SEQ ID NO: 4850; SEQ ID NO: 4851; SEQ ID NO: 4852; SEQ ID NO: 4853; SEQ ID NO: 4854; SEQ ID NO: 4855; SEQ ID NO: 4856; SEQ ID NO: 4857; SEQ ID NO: 4858; SEQ ID NO: 4859; SEQ ID NO: 4860; SEQ ID NO: 4861; SEQ ID NO: 4862; SEQ ID NO: 4863; SEQ ID NO: 4864; SEQ ID NO: 4865; SEQ ID NO: 9637; SEQ ID NO: 9638 and SEQ ID NO: 9639.

In another embodiment, the H. pylori cell envelope polypeptide or a fragment thereof is an H. pylori outer membrane polypeptide or a fragment thereof having an amino acid sequence selected from the group consisting of SEQ ID NO: 4866; SEQ ID NO: 4867; SEQ ID NO: 4868; SEQ ID NO: 4869; SEQ ID NO: 4870; SEQ ID NO: 4871; SEQ ID NO: 4872; SEQ ID NO: 4873; SEQ ID NO: 4874; SEQ ID NO: 4875; SEQ ID NO: 4876; SEQ ID NO: 4877; SEQ ID NO: 4878; SEQ ID NO: 4879; SEQ ID NO: 4880; SEQ ID NO: 4881; SEQ ID NO: 4882; SEQ ID NO: 4883; SEQ ID NO: 4884; SEQ ID NO: 4885; SEQ ID NO: 4886; SEQ ID NO: 4887; SEQ ID NO: 4888; SEQ ID NO: 4889; SEQ ID NO: 4890; SEQ ID NO: 4891; SEQ ID NO: 4892; SEQ ID NO: 4893; SEQ ID NO: 4894; SEQ ID NO: 4895; SEQ ID NO: 4896; SEQ ID NO: 4897; SEQ ID NO: 4898; SEQ ID NO: 4899; SEQ ID NO: 4900; SEQ ID NO: 4901; SEQ ID NO: 4902; SEQ ID NO: 4903; SEQ ID NO: 4904; SEQ ID NO: 4905; SEQ ID NO: 4906; SEQ ID NO: 4907; SEQ ID NO: 4908; SEQ ID NO: 4909; SEQ ID NO: 4910; SEQ ID NO: 4911; SEQ ID NO: 4912; SEQ ID NO: 4913; SEQ ID NO: 4914; SEQ ID NO: 4915; SEQ ID NO: 4916; SEQ ID NO: 4917; SEQ ID NO: 4918; SEQ ID NO: 4919; SEQ ID NO: 4920; SEQ ID NO: 4921; SEQ ID NO: 4922; SEQ ID NO: 4923; SEQ ID NO: 4924; SEQ ID NO: 4925; SEQ ID NO: 4926; SEQ ID NO: 4927; SEQ ID NO: 4928; SEQ ID NO: 4929; SEQ ID NO: 4930; SEQ ID NO: 4931; SEQ ID NO: 4932; SEQ ID NO: 4933; SEQ ID NO: 4934; SEQ ID NO: 4935; SEQ ID NO: 4936; SEQ ID NO: 4937; SEQ ID NO: 4938; SEQ ID NO: 4939; SEQ ID NO: 4940; SEQ ID NO: 4941; SEQ ID NO: 4942; SEQ ID NO: 4943; SEQ ID NO: 4944; SEQ ID NO: 4945; SEQ ID NO: 4946; SEQ ID NO: 4947; SEQ ID NO: 4948; SEQ ID NO: 4949; SEQ ID NO: 4950; SEQ ID NO: 4951; SEQ ID NO: 4952; SEQ ID NO: 4953; SEQ ID NO: 4954; SEQ ID NO: 4955; SEQ ID NO: 4956; SEQ ID NO: 4957; SEQ ID NO: 4958; SEQ ID NO: 4959; SEQ ID NO: 4960; SEQ ID NO: 4961; SEQ ID NO: 4962; SEQ ID NO: 4963; SEQ ID NO: 4964; SEQ ID NO: 4965; SEQ ID NO: 4966; SEQ ID NO: 4967; SEQ ID NO: 4968; SEQ ID NO: 4969; SEQ ID NO: 4970; SEQ ID NO: 4971; SEQ ID NO: 4972; SEQ ID NO: 4973; SEQ ID NO: 4974; SEQ ID NO: 4975; SEQ ID NO: 4976; SEQ ID NO: 4977; SEQ ID NO: 4978; SEQ ID NO: 4979; SEQ ID NO: 4980; SEQ ID NO: 4981; SEQ ID NO: 4982; SEQ ID NO: 4983; SEQ ID NO: 4984; SEQ ID NO: 4985; SEQ ID NO: 4986; SEQ ID NO: 4987; SEQ ID NO: 4988; SEQ ID NO: 4989; SEQ ID NO: 4990; SEQ ID NO: 4991; SEQ ID NO: 4992; SEQ ID NO: 4993; SEQ ID NO: 4994; SEQ ID NO: 4995; SEQ ID NO: 4996; SEQ ID NO: 4997; SEQ ID NO: 4998; SEQ ID NO: 4999; SEQ ID NO: 5000; SEQ ID NO: 5001; SEQ ID NO: 5002; SEQ ID NO: 5003; SEQ ID NO: 5004; SEQ ID NO: 5005; SEQ ID NO: 5006; SEQ ID NO: 5007; SEQ ID NO: 5008; SEQ ID NO: 5009; SEQ ID NO: 5010; SEQ ID NO: 5011; SEQ ID NO: 5012; SEQ ID NO: 5013; SEQ ID NO: 5014; SEQ ID NO: 5015; SEQ ID NO: 5016; SEQ ID NO: 5017; SEQ ID NO: 5018; SEQ ID NO: 5019; SEQ ID NO: 5020; SEQ ID NO: 5021; SEQ ID NO: 5022; SEQ ID NO: 5023; SEQ ID NO: 5024; SEQ ID NO: 5025; SEQ ID NO: 5026; SEQ ID NO: 5027; SEQ ID NO: 5028; SEQ ID NO: 5029; SEQ ID NO: 5030; SEQ ID NO: 5031; SEQ ID NO: 5032; SEQ ID NO: 5033; SEQ ID NO: 5034; SEQ ID NO: 5035; SEQ ID NO: 5036; SEQ ID NO: 5037; SEQ ID NO: 5038; SEQ ID NO: 5039; SEQ ID NO: 5040; SEQ ID NO: 5041; SEQ ID NO: 5042; SEQ ID NO: 5043; SEQ ID NO: 5044; SEQ ID NO: 5045; SEQ ID NO: 5046; SEQ ID NO: 5047; SEQ ID NO: 5048; SEQ ID NO: 5049; SEQ ID NO: 5050; SEQ ID NO: 5051; SEQ ID NO: 5052; SEQ ID NO: 5053; SEQ ID NO: 5054; SEQ ID NO: 5055; SEQ ID NO: 5056; SEQ ID NO: 5057; SEQ ID NO: 5058; SEQ ID NO: 5059; SEQ ID NO: 5060; SEQ ID NO: 5061; SEQ ID NO: 5062; SEQ ID NO: 5063; SEQ ID NO: 5064; SEQ ID NO: 5065; SEQ ID NO: 5066; SEQ ID NO: 5067; SEQ ID NO: 5068; SEQ ID NO: 5069; SEQ ID NO: 5070; SEQ ID NO: 5071; SEQ ID NO: 5072; SEQ ID NO: 5073; SEQ ID NO: 5074; SEQ ID NO: 5075; SEQ ID NO: 5076; SEQ ID NO: 5077; SEQ ID NO: 5078; SEQ ID NO: 5079; SEQ ID NO: 5080; SEQ ID NO: 5081; SEQ ID NO: 5082; SEQ ID NO: 5083; SEQ ID NO: 5084; SEQ ID NO: 5085; SEQ ID NO: 5086; SEQ ID NO: 5087; SEQ ID NO: 5088; SEQ ID NO: 5089; SEQ ID NO: 5090; SEQ ID NO: 5091; SEQ ID NO: 5092; SEQ ID NO: 5093; SEQ ID NO: 5094; SEQ ID NO: 5095; SEQ ID NO: 5096; SEQ ID NO: 5097; SEQ ID NO: 5098; SEQ ID NO: 5099; SEQ ID NO: 5100; SEQ ID NO: 5101; SEQ ID NO: 5102; SEQ ID NO: 5103; SEQ ID NO: 5104; SEQ ID NO: 5105; SEQ ID NO: 5106; SEQ ID NO: 5107; SEQ ID NO: 5108; SEQ ID NO: 5109; SEQ ID NO: 5110; SEQ ID NO: 5111; SEQ ID NO: 5112; SEQ ID NO: 5113; SEQ ID NO: 5114; SEQ ID NO: 5115; SEQ ID NO: 5116; SEQ ID NO: 5117; SEQ ID NO: 5118; SEQ ID NO: 5119; SEQ ID NO: 5120; SEQ ID NO: 5121; SEQ ID NO: 5122; SEQ ID NO: 5123; SEQ ID NO: 5124; SEQ ID NO: 5125; SEQ ID NO: 5126; SEQ ID NO: 5127; SEQ ID NO: 5128; SEQ ID NO: 5129; SEQ ID NO: 5130; SEQ ID NO: 5131; SEQ ID NO: 5132; SEQ ID NO: 5133; SEQ ID NO: 5134; SEQ ID NO: 5135; SEQ ID NO: 5136; SEQ ID NO: 5137; SEQ ID NO: 5138; SEQ ID NO: 5139; SEQ ID NO: 5140; SEQ ID NO: 5141; SEQ ID NO: 5142; SEQ ID NO: 5143; SEQ ID NO: 5144; SEQ ID NO: 5145; SEQ ID NO: 5146; SEQ ID NO: 5147; SEQ ID NO: 5148; SEQ ID NO: 5149; SEQ ID NO: 5150; SEQ ID NO: 5151; SEQ ID NO: 5152; SEQ ID NO: 5153; SEQ ID NO: 5154; SEQ ID NO: 5155; SEQ ID NO: 5156; SEQ ID NO: 5157; SEQ ID NO: 5158; SEQ ID NO: 5159; SEQ ID NO: 5160; SEQ ID NO: 5161; SEQ ID NO: 5162; SEQ ID NO: 5163; SEQ ID NO: 5164; SEQ ID NO: 5165; SEQ ID NO: 5166; SEQ ID NO: 5167; SEQ ID NO: 5168; SEQ ID NO: 5169; SEQ ID NO: 5170; SEQ ID NO: 5171; SEQ ID NO: 5172; SEQ ID NO: 5173; SEQ ID NO: 5174; SEQ ID NO: 5175; SEQ ID NO: 5176; SEQ ID NO: 5177; SEQ ID NO: 5178; SEQ ID NO: 5179; SEQ ID NO: 5180; SEQ ID NO: 5181; SEQ ID NO: 5182; SEQ ID NO: 5183; SEQ ID NO: 5184; SEQ ID NO: 5185; SEQ ID NO: 5186; SEQ ID NO: 5187; SEQ ID NO: 5188; SEQ ID NO: 5189; SEQ ID NO: 5190; SEQ ID NO: 5191; SEQ ID NO: 5192; SEQ ID NO: 5193; SEQ ID NO: 5194; SEQ ID NO: 5195; SEQ ID NO: 5196; SEQ ID NO: 5197; SEQ ID NO: 5198; SEQ ID NO: 5199; SEQ ID NO: 5200; SEQ ID NO: 5201; SEQ ID NO: 5202; SEQ ID NO: 5203; SEQ ID NO: 5204; SEQ ID NO: 5205; SEQ ID NO: 5206; SEQ ID NO: 5207; SEQ ID NO: 5208; SEQ ID NO: 5209; SEQ ID NO: 5210; SEQ ID NO: 5211; SEQ ID NO: 5212; SEQ ID NO: 5213; SEQ ID NO: 5214; SEQ ID NO: 5215; SEQ ID NO: 5216; SEQ ID NO: 5217; SEQ ID NO: 5218; SEQ ID NO: 5219; SEQ ID NO: 5220; SEQ ID NO: 5221; SEQ ID NO: 5222; SEQ ID NO: 5223; SEQ ID NO: 5224; SEQ ID NO: 5225; SEQ ID NO: 5226; SEQ ID NO: 5227; SEQ ID NO: 5228; SEQ ID NO: 5229; SEQ ID NO: 5230; SEQ ID NO: 5231; SEQ ID NO: 5232; SEQ ID NO: 5233; SEQ ID NO: 5234; SEQ ID NO: 5235; SEQ ID NO: 5236; SEQ ID NO: 5237; SEQ ID NO: 5238; SEQ ID NO: 5239; SEQ ID NO: 5240; SEQ ID NO: 5241; SEQ ID NO: 5242; SEQ ID NO: 5243; SEQ ID NO: 5244; SEQ ID NO: 5245; SEQ ID NO: 5246; SEQ ID NO: 5247; SEQ ID NO: 5248; SEQ ID NO: 5249; SEQ ID NO: 5250; SEQ ID NO: 5251; SEQ ID NO: 5252; SEQ ID NO: 5253; SEQ ID NO: 5254; SEQ ID NO: 5255; SEQ ID NO: 5256; SEQ ID NO: 5257; SEQ ID NO: 5258; SEQ ID NO: 5259; SEQ ID NO: 5260; SEQ ID NO: 5261; SEQ ID NO: 5262; SEQ ID NO: 5263; SEQ ID NO: 5264; SEQ ID NO: 5265; SEQ ID NO: 5266; SEQ ID NO: 5267; SEQ ID NO: 5268; SEQ ID NO: 5269; SEQ ID NO: 5270; SEQ ID NO: 5271 and SEQ ID NO: 5272.

In another embodiment, the H. pylori outer membrane polypeptide or a fragment thereof is an H. pylori polypeptide having a terminal phenylalanine residue or a fragment thereof having an amino acid sequence selected from the group consisting of SEQ ID NO: 4866; SEQ ID NO: 4867; SEQ ID NO: 4868; SEQ ID NO: 4869; SEQ ID NO: 4870; SEQ ID NO: 4871; SEQ ID NO: 4872; SEQ ID NO: 4873; SEQ ID NO: 4874; SEQ ID NO: 4875; SEQ ID NO: 4876; SEQ ID NO: 4877; SEQ ID NO: 4878; SEQ ID NO: 4879; SEQ ID NO: 4880; SEQ ID NO: 4881; SEQ ID NO: 4882; SEQ ID NO: 4883; SEQ ID NO: 4884; SEQ ID NO: 4885; SEQ ID NO: 4886; SEQ ID NO: 4887; SEQ ID NO: 4888; SEQ ID NO: 4889; SEQ ID NO: 4890; SEQ ID NO: 4891; SEQ ID NO: 4892; SEQ ID NO: 4893; SEQ ID NO: 4894; SEQ ID NO: 4895; SEQ ID NO: 4896; SEQ ID NO: 4897; SEQ ID NO: 4898; SEQ ID NO: 4899; SEQ ID NO: 4900; SEQ ID NO: 4901; SEQ ID NO: 4902; SEQ ID NO: 4903; SEQ ID NO: 4904; SEQ ID NO: 4905; SEQ ID NO: 4906; SEQ ID NO: 4907; SEQ ID NO: 4908; SEQ ID NO: 4909; SEQ ID NO: 4910; SEQ ID NO: 4911; SEQ ID NO: 4912; SEQ ID NO: 4913; SEQ ID NO: 4914; SEQ ID NO: 4915; SEQ ID NO: 4916; SEQ ID NO: 4917; SEQ ID NO: 4918; SEQ ID NO: 4919; SEQ ID NO: 4920; SEQ ID NO: 4921; SEQ ID NO: 4922; SEQ ID NO: 4923; SEQ ID NO: 4924; SEQ ID NO: 4925; SEQ ID NO: 4926; SEQ ID NO: 4927; SEQ ID NO: 4928; SEQ ID NO: 4929; SEQ ID NO: 4930; SEQ ID NO: 4931; SEQ ID NO: 4932; SEQ ID NO: 4933; SEQ ID NO: 4934; SEQ ID NO: 4935; SEQ ID NO: 4936; SEQ ID NO: 4937; SEQ ID NO: 4938; SEQ ID NO: 4939; SEQ ID NO: 4940; SEQ ID NO: 4941; SEQ ID NO: 4942; SEQ ID NO: 4943; SEQ ID NO: 4944; SEQ ID NO: 4945; SEQ ID NO: 4946; SEQ ID NO: 4947; SEQ ID NO: 4948; SEQ ID NO: 4949; SEQ ID NO: 4950; SEQ ID NO: 4951; SEQ ID NO: 4952; SEQ ID NO: 4953; SEQ ID NO: 4954; SEQ ID NO: 4955; SEQ ID NO: 4956; SEQ ID NO: 4957; SEQ ID NO: 4958; SEQ ID NO: 4959; SEQ ID NO: 4960; SEQ ID NO: 4961; SEQ ID NO: 4962; SEQ ID NO: 4963; SEQ ID NO: 4964; SEQ ID NO: 4965; SEQ ID NO: 4966; SEQ ID NO: 4967; SEQ ID NO: 4968; SEQ ID NO: 4969; SEQ ID NO: 4970; SEQ ID NO: 4971; SEQ ID NO: 4972; SEQ ID NO: 4973; SEQ ID NO: 4974; SEQ ID NO: 4975; SEQ ID NO: 4976; SEQ ID NO: 4977; SEQ ID NO: 4978; SEQ ID NO: 4979; SEQ ID NO: 4980; SEQ ID NO: 4981; SEQ ID NO: 4982; SEQ ID NO: 4983; SEQ ID NO: 4984; SEQ ID NO: 4985; SEQ ID NO: 4986; SEQ ID NO: 4987; SEQ ID NO: 4988; SEQ ID NO: 4989; SEQ ID NO: 4990; SEQ ID NO: 4991; SEQ ID NO: 4992; SEQ ID NO: 4993; SEQ ID NO: 4994; SEQ ID NO: 4995; SEQ ID NO: 4996; SEQ ID NO: 4997; SEQ ID NO: 4998; SEQ ID NO: 4999; SEQ ID NO: 5000; SEQ ID NO: 5001; SEQ ID NO: 5002; SEQ ID NO: 5003; SEQ ID NO: 5004; SEQ ID NO: 5005; SEQ ID NO: 5006; SEQ ID NO: 5007; SEQ ID NO: 5008; SEQ ID NO: 5009; SEQ ID NO: 5010; SEQ ID NO: 5011; SEQ ID NO: 5012; SEQ ID NO: 5013; SEQ ID NO: 5014; SEQ ID NO: 5015; SEQ ID NO: 5016; SEQ ID NO: 5017; SEQ ID NO: 5018; SEQ ID NO: 5019; SEQ ID NO: 5020; SEQ ID NO: 5021; SEQ ID NO: 5022; SEQ ID NO: 5023; SEQ ID NO: 5024; SEQ ID NO: 5025; SEQ ID NO: 5026; SEQ ID NO: 5027; SEQ ID NO: 5028; SEQ ID NO: 5029; SEQ ID NO: 5030; SEQ ID NO: 5031; SEQ ID NO: 5032; SEQ ID NO: 5033; SEQ ID NO: 5034; SEQ ID NO: 5035; SEQ ID NO: 5036; SEQ ID NO: 5037; SEQ ID NO: 5038; SEQ ID NO: 5039; SEQ ID NO: 5040; SEQ ID NO: 5041; SEQ ID NO: 5042; SEQ ID NO: 5043; SEQ ID NO: 5044; SEQ ID NO: 5045; SEQ ID NO: 5046; SEQ ID NO: 5047; SEQ ID NO: 5048; SEQ ID NO: 5049; SEQ ID NO: 5050; SEQ ID NO: 5051; SEQ ID NO: 5052; SEQ ID NO: 5053; SEQ ID NO: 5054; SEQ ID NO: 5055; SEQ ID NO: 5056; SEQ ID NO: 5057; SEQ ID NO: 5058; SEQ ID NO: 5059; SEQ ID NO: 5060; SEQ ID NO: 5061; SEQ ID NO: 5062; SEQ ID NO: 5063; SEQ ID NO: 5064; SEQ ID NO: 5065; SEQ ID NO: 5066; SEQ ID NO: 5067; SEQ ID NO: 5068; SEQ ID NO: 5069; SEQ ID NO: 5070; SEQ ID NO: 5071; SEQ ID NO: 5072; SEQ ID NO: 5073; SEQ ID NO: 5074; SEQ ID NO: 5075; SEQ ID NO: 5076; SEQ ID NO: 5077; SEQ ID NO: 5078; SEQ ID NO: 5079; SEQ ID NO: 5080; SEQ ID NO: 5081; SEQ ID NO: 5082; SEQ ID NO: 5083; SEQ ID NO: 5084; SEQ ID NO: 5085; SEQ ID NO: 5086; SEQ ID NO: 5087; SEQ ID NO: 5088; SEQ ID NO: 5089; SEQ ID NO: 5090; SEQ ID NO: 5091; SEQ ID NO: 5092; SEQ ID NO: 5093; SEQ ID NO: 5094; SEQ ID NO: 5095; SEQ ID NO: 5104; SEQ ID NO: 5105; SEQ ID NO: 5106; SEQ ID NO: 5107; SEQ ID NO: 5108; SEQ ID NO: 5109; SEQ ID NO: 5110; SEQ ID NO: 5111; SEQ ID NO: 5112; SEQ ID NO: 5113; SEQ ID NO: 5114; SEQ ID NO: 5115; SEQ ID NO: 5116; SEQ ID NO: 5117; SEQ ID NO: 5118; SEQ ID NO: 5119; SEQ ID NO: 5120; SEQ ID NO: 5121; SEQ ID NO: 5122; SEQ ID NO: 5123; SEQ ID NO: 5124; SEQ ID NO: 5131; SEQ ID NO: 5132; SEQ ID NO: 5133; SEQ ID NO: 5134; SEQ ID NO: 5135; SEQ ID NO: 5136; SEQ ID NO: 5137; SEQ ID NO: 5138; SEQ ID NO: 5139 SEQ ID NO: 5140; SEQ ID NO: 5141; SEQ ID NO: 5142; SEQ ID NO: 5143; SEQ ID NO: 5144; SEQ ID NO: 5145; SEQ ID NO: 5146; SEQ ID NO: 5147; SEQ ID NO: 5148; SEQ ID NO: 5149; SEQ ID NO: 5150; SEQ ID NO: 5151; SEQ ID NO: 5152; SEQ ID NO: 5153; SEQ ID NO: 5154; SEQ ID NO: 5155; SEQ ID NO: 5156; SEQ ID NO: 5157; SEQ ID NO: 5158; SEQ ID NO: 5159; SEQ ID NO: 9640; SEQ ID NO: 9641; SEQ ID NO: 9642; SEQ ID NO: 9643; SEQ ID NO: 9644; SEQ ID NO: 9645; SEQ ID NO: 9646; SEQ ID NO: 9647 and SEQ ID NO: 9648.

In another embodiment, the H. pylori outer membrane polypeptide or a fragment thereof is an H. pylori polypeptide having a terminal phenylalanine residue and a C-terminal tyrosine cluster or a fragment thereof having an amino acid sequence selected from the group consisting of SEQ ID NO: 5088; SEQ ID NO: 5089; SEQ ID NO: 5090; SEQ ID NO: 5091; SEQ ID NO: 5092; SEQ ID NO: 5093; SEQ ID NO: 5094; SEQ ID NO: 5095; SEQ ID NO: 5104; SEQ ID NO: 5105; SEQ ID NO: 5106; SEQ ID NO: 5107; SEQ ID NO: 5108; SEQ ID NO: 5109; SEQ ID NO: 5110; SEQ ID NO: 5111; SEQ ID NO: 5112; SEQ ID NO: 5113; SEQ ID NO: 5114; SEQ ID NO: 5115; SEQ ID NO: 5116; SEQ ID NO: 5117; SEQ ID NO: 5118; SEQ ID NO: 5119; SEQ ID NO: 5120; SEQ ID NO: 5121; SEQ ID NO: 5122; SEQ ID NO: 5123; SEQ ID NO: 5124; SEQ ID NO: 5131; SEQ ID NO: 5132; SEQ ID NO: 5133; SEQ ID NO: 5134; SEQ ID NO: 5135; SEQ ID NO: 5136; SEQ ID NO: 5137; SEQ ID NO: 5138; SEQ ID NO: 5139; SEQ ID NO: 5140; SEQ ID NO: 5141; SEQ ID NO: 5142; SEQ ID NO: 5143; SEQ ID NO: 5144; SEQ ID NO: 5145; SEQ ID NO: 5146; SEQ ID NO: 5147; SEQ ID NO: 5148; SEQ ID NO: 5149; SEQ ID NO: 5150; SEQ ID NO: 5151; SEQ ID NO: 5152; SEQ ID NO: 5153; SEQ ID NO: 5154; SEQ ID NO: 5155; SEQ ID NO: 5156; SEQ ID NO: 5157; SEQ ID NO: 5158; SEQ ID NO: 5159 and SEQ ID NO: 9648.

In another embodiment, the H. pylori outer membrane polypeptide or a fragment thereof is an H. pylori polypeptide having a terminal tyrosine cluster motif or a fragment thereof having an amino acid sequence selected from the group consisting of SEQ ID NO: 5088; SEQ ID NO: 5089; SEQ ID NO: 5090; SEQ ID NO: 5091; SEQ ID NO: 5092; SEQ ID NO: 5093; SEQ ID NO: 5094; SEQ ID NO: 5095; SEQ ID NO: 5096; SEQ ID NO: 5097; SEQ ID NO: 5098; SEQ ID NO: 5099; SEQ ID NO: 5100; SEQ ID NO: 5101; SEQ ID NO: 5102; SEQ ID NO: 5103; SEQ ID NO: 5104; SEQ ID NO: 5105; SEQ ID NO: 5106; SEQ ID NO: 5107; SEQ ID NO: 5108; SEQ ID NO: 5109; SEQ ID NO: 5110; SEQ ID NO: 5111; SEQ ID NO: 5112; SEQ ID NO: 5113; SEQ ID NO: 5114; SEQ ID NO: 5115; SEQ ID NO: 5116; SEQ ID NO: 5117; SEQ ID NO: 5118; SEQ ID NO: 5119; SEQ ID NO: 5120; SEQ ID NO: 5121; SEQ ID NO: 5122; SEQ ID NO: 5123; SEQ ID NO: 5124; SEQ ID NO: 5125; SEQ ID NO: 5126; SEQ ID NO: 5127; SEQ ID NO: 5128; SEQ ID NO: 5129; SEQ ID NO: 5130; SEQ ID NO: 5131; SEQ ID NO: 5132; SEQ ID NO: 5133; SEQ ID NO: 5134; SEQ ID NO: 5135; SEQ ID NO: 5136; SEQ ID NO: 5137; SEQ ID NO: 5138; SEQ ID NO: 5139; SEQ ID NO: 5140; SEQ ID NO: 5141; SEQ ID NO: 5142; SEQ ID NO: 5143; SEQ ID NO: 5144; SEQ ID NO: 5145; SEQ ID NO: 5146; SEQ ID NO: 5147; SEQ ID NO: 5148; SEQ ID NO: 5149; SEQ ID NO: 5150; SEQ ID NO: 5151; SEQ ID NO: 5152; SEQ ID NO: 5153; SEQ ID NO: 5154; SEQ ID NO: 5155; SEQ ID NO: 5156; SEQ ID NO: 5157; SEQ ID NO: 5158; SEQ ID NO: 5159; SEQ ID NO: 5160; SEQ ID NO: 5161; SEQ ID NO: 5162; SEQ ID NO: 5163; SEQ ID NO: 5164; SEQ ID NO: 5165; SEQ ID NO: 5166; SEQ ID NO: 5167; SEQ ID NO: 5168; SEQ ID NO: 5169; SEQ ID NO: 5170; SEQ ID NO: 5171; SEQ ID NO: 5172; SEQ ID NO: 5173; SEQ ID NO: 5174; SEQ ID NO: 5175; SEQ ID NO: 5176; SEQ ID NO: 5177; SEQ ID NO: 5178; SEQ ID NO: 5186; SEQ ID NO: 5187; and SEQ ID NO: 9648.

In another embodiment, the H. pylori cell envelope polypeptide or a fragment thereof is an H. pylori inner membrane polypeptide or a fragment thereof having an amino acid sequence selected from the group consisting of SEQ ID NO: 5273; SEQ ID NO: 5274; SEQ ID NO: 5275; SEQ ID NO: 5276; SEQ ID NO: 5277; SEQ ID NO: 5278; SEQ ID NO: 5279; SEQ ID NO: 5280; SEQ ID NO: 5281; SEQ ID NO: 5282; SEQ ID NO: 5283; SEQ ID NO: 5284; SEQ ID NO: 5285; SEQ ID NO: 5286; SEQ ID NO: 5287; SEQ ID NO: 5288; SEQ ID NO: 5289; SEQ ID NO: 5290; SEQ ID NO: 5291; SEQ ID NO: 5292; SEQ ID NO: 5293; SEQ ID NO: 5294; SEQ ID NO: 5295; SEQ ID NO: 5296; SEQ ID NO: 5297; SEQ ID NO: 5298; SEQ ID NO: 5299; SEQ ID NO: 5300; SEQ ID NO: 5301; SEQ ID NO: 5302; SEQ ID NO: 5303; SEQ ID NO: 5304; SEQ ID NO: 5305; SEQ ID NO: 5306; SEQ ID NO: 5307; SEQ ID NO: 5308; SEQ ID NO: 5309; SEQ ID NO: 5310; SEQ ID NO: 5311; SEQ ID NO: 5312; SEQ ID NO: 5313; SEQ ID NO: 5314; SEQ ID NO: 5315; SEQ ID NO: 5316; SEQ ID NO: 5317; SEQ ID NO: 5318; SEQ ID NO: 5319; SEQ ID NO: 5320; SEQ ID NO: 5321; SEQ ID NO: 5322; SEQ ID NO: 5323; SEQ ID NO: 5324; SEQ ID NO: 5325; SEQ ID NO: 5326; SEQ ID NO: 5327; SEQ ID NO: 5328; SEQ ID NO: 5329; SEQ ID NO: 5330; SEQ ID NO: 5331; SEQ ID NO: 5332; SEQ ID NO: 5333; SEQ ID NO: 5334; SEQ ID NO: 5335; SEQ ID NO: 5336; SEQ ID NO: 5337; SEQ ID NO: 5338; SEQ ID NO: 5339; SEQ ID NO: 5340; SEQ ID NO: 5341; SEQ ID NO: 5342; SEQ ID NO: 5343; SEQ ID NO: 5344; SEQ ID NO: 5345; SEQ ID NO: 5346; SEQ ID NO: 5347; SEQ ID NO: 5348; SEQ ID NO: 5349; SEQ ID NO: 5350; SEQ ID NO: 5351; SEQ ID NO: 5352; SEQ ID NO: 5353; SEQ ID NO: 5354; SEQ ID NO: 5355; SEQ ID NO: 5356; SEQ ID NO: 5357; SEQ ID NO: 5358; SEQ ID NO: 5359; SEQ ID NO: 5360; SEQ ID NO: 5361; SEQ ID NO: 5362; SEQ ID NO: 5363; SEQ ID NO: 5364; SEQ ID NO: 5365; SEQ ID NO: 5366; SEQ ID NO: 5367; SEQ ID NO: 5368; SEQ ID NO: 5369; SEQ ID NO: 5370; SEQ ID NO: 5371; SEQ ID NO: 5372; SEQ ID NO: 5373; SEQ ID NO: 5374; SEQ ID NO: 5375; SEQ ID NO: 5376; SEQ ID NO: 5377; SEQ ID NO: 5378; SEQ ID NO: 5379; SEQ ID NO: 5380; SEQ ID NO: 5381; SEQ ID NO: 5382; SEQ ID NO: 5383; SEQ ID NO: 5384; SEQ ID NO: 5385; SEQ ID NO: 5386; SEQ ID NO: 5387; SEQ ID NO: 5388; SEQ ID NO: 5389; SEQ ID NO: 5390; SEQ ID NO: 5391; SEQ ID NO: 5392; SEQ ID NO: 5393; SEQ ID NO: 5394; SEQ ID NO: 5395; SEQ ID NO: 5396; SEQ ID NO: 5397; SEQ ID NO: 5398; SEQ ID NO: 5399; SEQ ID NO: 5400; SEQ ID NO: 5401; SEQ ID NO: 5402; SEQ ID NO: 5403; SEQ ID NO: 5404; SEQ ID NO: 5405; SEQ ID NO: 5406; SEQ ID NO: 5407; SEQ ID NO: 5408; SEQ ID NO: 5409; SEQ ID NO: 5410; SEQ ID NO: 5411; SEQ ID NO: 5412; SEQ ID NO: 5413; SEQ ID NO: 5414; SEQ ID NO: 5415; SEQ ID NO: 5416; SEQ ID NO: 5417; SEQ ID NO: 5418; SEQ ID NO: 5419; SEQ ID NO: 5420; SEQ ID NO: 5421; SEQ ID NO: 5422; SEQ ID NO: 5423; SEQ ID NO: 5424; SEQ ID NO: 5425; SEQ ID NO: 5426; SEQ ID NO: 5427; SEQ ID NO: 5428; SEQ ID NO: 5429; SEQ ID NO: 5430; SEQ ID NO: 5431; SEQ ID NO: 5432; SEQ ID NO: 5433; SEQ ID NO: 5434; SEQ ID NO: 5435; SEQ ID NO: 5436; SEQ ID NO: 5437; SEQ ID NO: 5438; SEQ ID NO: 5439; SEQ ID NO: 5440; SEQ ID NO: 5441; SEQ ID NO: 5442; SEQ ID NO: 5443; SEQ ID NO: 5444; SEQ ID NO: 5445; SEQ ID NO: 5446; SEQ ID NO: 5447; SEQ ID NO: 5448; SEQ ID NO: 5449; SEQ ID NO: 5450; SEQ ID NO: 5451; SEQ ID NO: 5452; SEQ ID NO: 5453; SEQ ID NO: 5454; SEQ ID NO: 5455; SEQ ID NO: 5456; SEQ ID NO: 5457; SEQ ID NO: 5458; SEQ ID NO: 5459; SEQ ID NO: 5460; SEQ ID NO: 5461; SEQ ID NO: 5462; SEQ ID NO: 5463; SEQ ID NO: 5464; SEQ ID NO: 5465; SEQ ID NO: 5466; SEQ ID NO:5467; SEQ ID NO: 5468; SEQ ID NO: 5469; SEQ ID NO: 5470; SEQ ID NO: 5471; SEQ ID NO: 5472; SEQ ID NO: 5473; SEQ ID NO: 5474; SEQ ID NO: 5475; SEQ ID NO: 5476; SEQ ID NO: 5477; SEQ ID NO: 5478; SEQ ID NO: 5479; SEQ ID NO: 5480; SEQ ID NO: 5481; SEQ ID NO: 5482; SEQ ID NO: 5483; SEQ ID NO: 5484; SEQ ID NO: 5485; SEQ ID NO: 5486; SEQ ID NO: 5487; SEQ ID NO: 5488; SEQ ID NO: 5489; SEQ ID NO: 5490; SEQ ID NO: 5491; SEQ ID NO: 5492; SEQ ID NO: 5493; SEQ ID NO: 5494; SEQ ID NO: 5495; SEQ ID NO: 5496; SEQ ID NO: 5497; SEQ ID NO: 5498; SEQ ID NO: 5499; SEQ ID NO: 5500; SEQ ID NO: 5501; SEQ ID NO: 5502; SEQ ID NO: 5503; SEQ ID NO: 5504; SEQ ID NO: 5505; SEQ ID NO: 5506; SEQ ID NO: 5507; SEQ ID NO: 5508; SEQ ID NO: 5509; SEQ ID NO: 5510; SEQ ID NO: 5511; SEQ ID NO: 5512; SEQ ID NO: 5513; SEQ ID NO: 5514; SEQ ID NO: 5515; SEQ ID NO: 5516; SEQ ID NO: 5517; SEQ ID NO: 5518; SEQ ID NO: 5519; SEQ ID NO: 5520; SEQ ID NO: 5521; SEQ ID NO: 5522; SEQ ID NO: 5523; SEQ ID NO: 5524; SEQ ID NO: 5525; SEQ ID NO: 5526; SEQ ID NO: 5527; SEQ ID NO: 5528; SEQ ID NO: 5529; SEQ ID NO: 5530; SEQ ID NO: 5531; SEQ ID NO: 5532; SEQ ID NO: 5533; SEQ ID NO: 5534; SEQ ID NO: 5535; SEQ ID NO: 5536; SEQ ID NO: 5537; SEQ ID NO: 5538; SEQ ID NO: 5539; SEQ ID NO: 5540; SEQ ID NO: 5541; SEQ ID NO: 5542; SEQ ID NO: 5543; SEQ ID NO: 5544; SEQ ID NO: 5545; SEQ ID NO: 5546; SEQ ID NO: 5547; SEQ ID NO: 5548; SEQ ID NO: 5549; SEQ ID NO: 5550; SEQ ID NO: 5551; SEQ ID NO: 5552; SEQ ID NO: 5553; SEQ ID NO: 5554; SEQ ID NO: 5555; SEQ ID NO: 5556; SEQ ID NO: 5557; SEQ ID NO: 5558; SEQ ID NO: 5559; SEQ ID NO: 5560; SEQ ID NO: 5561; SEQ ID NO: 5562; SEQ ID NO: 5563; SEQ ID NO: 5564; SEQ ID NO: 5565; SEQ ID NO: 5566; SEQ ID NO: 5567; SEQ ID NO: 5568; SEQ ID NO: 5569; SEQ ID NO: 5570; SEQ ID NO: 5571; SEQ ID NO: 5572; SEQ ID NO: 5573; SEQ ID NO: 5574; SEQ ID NO: 5575; SEQ ID NO: 5576; SEQ ID NO: 5577; SEQ ID NO: 5578; SEQ ID NO: 5579; SEQ ID NO: 5580; SEQ ID NO: 5581; SEQ ID NO: 5582; SEQ ID NO: 5583; SEQ ID NO: 5584; SEQ ID NO: 5585; SEQ ID NO: 5586; SEQ ID NO: 5587; SEQ ID NO: 5588; SEQ ID NO: 5589; SEQ ID NO: 5590; SEQ ID NO: 5591; SEQ ID NO: 5592; SEQ ID NO: 5593; SEQ ID NO: 5594; SEQ ID NO: 5595; SEQ ID NO: 5596; SEQ ID NO: 5597; SEQ ID NO: 5598; SEQ ID NO: 5599; SEQ ID NO: 5600; SEQ ID NO: 5601; SEQ ID NO: 5602; SEQ ID NO: 5603; SEQ ID NO: 5604; SEQ ID NO: 5605; SEQ ID NO: 5606; SEQ ID NO: 5607; SEQ ID NO: 5608; SEQ ID NO: 5609; SEQ ID NO: 5610; SEQ ID NO: 5611; SEQ ID NO: 5612; SEQ ID NO: 5613; SEQ ID NO: 5614; SEQ ID NO: 5615; SEQ ID NO: 5616; SEQ ID NO: 5617; SEQ ID NO: 5618; SEQ ID NO: 5619; SEQ ID NO: 5620; SEQ ID NO: 5621; SEQ ID NO: 5622; SEQ ID NO: 5623; SEQ ID NO: 5624; SEQ ID NO: 5625; SEQ ID NO: 5626; SEQ ID NO: 5627; SEQ ID NO: 5628; SEQ ID NO: 5629; SEQ ID NO: 5630; SEQ ID NO: 5631; SEQ ID NO: 5632; SEQ ID NO: 5633; SEQ ID NO: 5634; SEQ ID NO: 5635; SEQ ID NO: 5636; SEQ ID NO: 5637; SEQ ID NO: 5638; SEQ ID NO: 5639; SEQ ID NO: 5640; SEQ ID NO: 5641; SEQ ID NO: 5642; SEQ ID NO: 5643; SEQ ID NO: 5644; SEQ ID NO: 5645; SEQ ID NO: 5646; SEQ ID NO: 5647; SEQ ID NO: 5648; SEQ ID NO: 5649; SEQ ID NO: 5650; SEQ ID NO: 5651; SEQ ID NO: 5652; SEQ ID NO: 5653; SEQ ID NO: 5654; SEQ ID NO: 5655; SEQ ID NO: 5656; SEQ ID NO: 5657; SEQ ID NO: 5658; SEQ ID NO: 5659; SEQ ID NO: 5660; SEQ ID NO: 5661; SEQ ID NO: 5662; SEQ ID NO: 5663; SEQ ID NO: 5664; SEQ ID NO: 5665; SEQ ID NO: 5666; SEQ ID NO: 5667; SEQ ID NO: 5668; SEQ ID NO: 5669; SEQ ID NO: 5670; SEQ ID NO: 5671; SEQ ID NO: 5672; SEQ ID NO: 5673; SEQ ID NO: 5674; SEQ ID NO: 5675; SEQ ID NO: 5676; SEQ ID NO: 5677; SEQ ID NO: 5678; SEQ ID NO: 5679; SEQ ID NO: 5680; SEQ ID NO: 5681; SEQ ID NO: 5682; SEQ ID NO: 5683; SEQ ID NO: 5684; SEQ ID NO: 5685; SEQ ID NO: 5686; SEQ ID NO: 5687; SEQ ID NO: 5688; SEQ ID NO: 5689; SEQ ID NO: 5690; SEQ ID NO: 5691; SEQ ID NO: 5692; SEQ ID NO: 5693; SEQ ID NO: 5694; SEQ ID NO: 5695; SEQ ID NO: 5.696; SEQ ID NO: 5697; SEQ ID NO: 5698; SEQ ID NO: 5699; SEQ ID NO: 5700; SEQ ID NO: 5701; SEQ ID NO: 5702; SEQ ID NO: 5703; SEQ ID NO: 5704; SEQ ID NO: 5705; SEQ ID NO: 5706; SEQ ID NO: 5707; SEQ ID NO: 5708; SEQ ID NO: 5709; SEQ ID NO: 5710; SEQ ID NO: 5711; SEQ ID NO: 5712; SEQ ID NO: 5713; SEQ ID NO: 5714; SEQ ID NO: 5715; SEQ ID NO: 5716; SEQ ID NO: 5717; SEQ ID NO: 5718; SEQ ID NO: 5719; SEQ ID NO: 5720; SEQ ID NO: 57-21; SEQ ID NO: 5722; SEQ ID NO: 5723; SEQ ID NO: 5724; SEQ ID NO: 5725; SEQ ID NO: 5726; SEQ ID NO: 5727; SEQ ID NO: 5728; SEQ ID NO: 5729; SEQ ID NO: 5730; SEQ ID NO: 5731; SEQ ID NO: 5732; SEQ ID NO: 5733; SEQ ID NO: 5734; SEQ ID NO: 5735; SEQ ID NO: 5736; SEQ ID NO: 5737; SEQ ID NO: 5738; SEQ ID NO: 5739; SEQ ID NO: 5740; SEQ ID NO: 5741; SEQ ID NO: 5742; SEQ ID NO: 5743; SEQ ID NO: 5744; SEQ ID NO: 5745; SEQ ID NO: 5746; SEQ ID NO: 5747; SEQ ID NO: 5748; SEQ ID NO: 5749; SEQ ID NO: 5750; SEQ ID NO: 5751; SEQ ID NO: 5752; SEQ ID NO: 5753; SEQ ID NO: 5754; SEQ ID NO: 5755; SEQ ID NO: 5756; SEQ ID NO: 5757; SEQ ID NO: 5758; SEQ ID NO: 5759; SEQ ID NO: 5760; SEQ ID NO: 5761; SEQ ID NO: 5762; SEQ ID NO: 5763; SEQ ID NO: 5764; SEQ ID NO: 5765; SEQ ID NO: 5766; SEQ ID NO: 5767; SEQ ID NO: 5768; SEQ ID NO: 5769; SEQ ID NO: 5770; SEQ ID NO: 5771; SEQ ID NO: 5772; SEQ ID NO: 5773; SEQ ID NO: 5774; SEQ ID NO: 5775; SEQ ID NO: 5776; SEQ ID NO: 5777; SEQ ID NO: 5778; SEQ ID NO: 5779; SEQ ID NO: 5780; SEQ ID NO: 5781; SEQ ID NO: 5782; SEQ ID NO: 5783; SEQ ID NO: 5784; SEQ ID NO: 5785; SEQ ID NO: 5786; SEQ ID NO: 5787; SEQ ID NO: 5788; SEQ ID NO: 5789; SEQ ID NO: 5790; SEQ ID NO: 5791; SEQ ID NO: 5792; SEQ ID NO: 5793; SEQ ID NO: 5794; SEQ ID NO: 5795; SEQ ID NO: 5796; SEQ ID NO: 5797; SEQ ID NO: 5798; SEQ ID NO: 5799; SEQ ID NO: 5800; SEQ ID NO: 5801; SEQ ID NO: 5802; SEQ ID NO: 5803; SEQ ID NO: 5804; SEQ ID NO: 5805; SEQ ID NO: 5806; SEQ ID NO: 5807; SEQ ID NO: 5808; SEQ ID NO: 5809; SEQ ID NO: 5810; SEQ ID NO: 5811; SEQ ID NO: 5812; SEQ ID NO: 5813; SEQ ID NO: 5814; SEQ ID NO: 5815; SEQ ID NO: 5816; SEQ ID NO: 5817; SEQ ID NO: 5818; SEQ ID NO: 5819; SEQ ID NO: 5820; SEQ ID NO: 5821; SEQ ID NO: 5822; SEQ ID NO: 5823; SEQ ID NO: 5824; SEQ ID NO: 5825; SEQ ID NO: 5826; SEQ ID NO: 5827; SEQ ID NO: 5828; SEQ ID NO: 5829; SEQ ID NO: 5830; SEQ ID NO: 5831; SEQ ID NO: 5832; SEQ ID NO: 5833; SEQ ID NO: 5834; SEQ ID NO: 5835; SEQ ID NO: 5836; SEQ ID NO: 5837; SEQ ID NO: 5838; SEQ ID NO: 5839; SEQ ID NO: 5840; SEQ ID NO: 5841; SEQ ID NO: 5842; SEQ ID NO: 5843; SEQ ID NO: 5844; SEQ ID NO: 5845; SEQ ID NO: 5846; SEQ ID NO: 5847; SEQ ID NO: 5848; SEQ ID NO: 5849; SEQ ID NO: 5850; SEQ ID NO: 5851; SEQ ID NO: 5852; SEQ ID NO: 5853; SEQ ID NO: 5854; SEQ ID NO: 5855; SEQ ID NO: 5856; SEQ ID NO: 5857; SEQ ID NO: 5858; SEQ ID NO: 5859; SEQ ID NO: 5860; SEQ ID NO: 5861; SEQ ID NO: 5862; SEQ ID NO: 5863; SEQ ID NO: 5864; SEQ ID NO: 5865; SEQ ID NO: 5866; SEQ ID NO: 5867; SEQ ID NO: 5868; SEQ ID NO: 5869; SEQ ID NO: 5870; SEQ ID NO: 5871; SEQ ID NO: 5872; SEQ ID NO: 5873; SEQ ID NO: 5874; SEQ ID NO: 5875; SEQ ID NO: 5876; SEQ ID NO: 5877; SEQ ID NO: 5878; SEQ ID NO: 5879; SEQ ID NO: 5880; SEQ ID NO: 5881; SEQ ID NO: 5882; SEQ ID NO: 5883; SEQ ID NO: 5884; SEQ ID NO: 5885; SEQ ID NO: 5886; SEQ ID NO: 5887; SEQ ID NO: 5888; SEQ ID NO: 5889; SEQ ID NO: 5890; SEQ ID NO: 5891; SEQ ID NO: 5892; SEQ ID NO: 5893; SEQ ID NO: 5894; SEQ ID NO: 5895; SEQ ID NO: 5896; SEQ ID NO: 5897; SEQ ID NO: 5898; SEQ ID NO: 5899; SEQ ID NO: 5900; SEQ ID NO: 5901; SEQ ID NO: 5902; SEQ ID NO: 5903; SEQ ID NO: 5904; SEQ ID NO: 5905; SEQ ID NO: 5906; SEQ ID NO: 5907; SEQ ID NO: 5908; SEQ ID NO: 5909; SEQ ID NO: 5910; SEQ ID NO: 5911; SEQ ID NO: 5912; SEQ ID NO: 5913; SEQ ID NO: 5914; SEQ ID NO: 5915; SEQ ID NO: 5916; SEQ ID NO: 5917; SEQ ID NO: 5918; SEQ ID NO: 5919; SEQ ID NO: 5920; SEQ ID NO: 5921; SEQ ID NO: 5922; SEQ ID NO: 5923; SEQ ID NO: 5924; SEQ ID NO: 5925; SEQ ID NO: 5926; SEQ ID NO: 5927; SEQ ID NO: 5928; SEQ ID NO: 5929; SEQ ID NO: 5930; SEQ ID NO: 5931; SEQ ID NO: 5932; SEQ ID NO: 5933; SEQ ID NO: 5934; SEQ ID NO: 5935; SEQ ID NO: 5936; SEQ ID NO: 5937; SEQ ID NO: 5938; SEQ ID NO: 5939; SEQ ID NO: 5940; SEQ ID NO: 5941; SEQ ID NO: 5942; SEQ ID NO: 5943; SEQ ID NO: 5944; SEQ ID NO: 5945; SEQ ID NO: 5946; SEQ ID NO: 5947; SEQ ID NO: 5948; SEQ ID NO: 5949; SEQ ID NO: 5950; SEQ ID NO: 5951; SEQ ID NO: 5952; SEQ ID NO: 5953; SEQ ID NO: 5954; SEQ ID NO: 5955; SEQ ID NO: 5956; SEQ ID NO: 5957; SEQ ID NO: 5958; SEQ ID NO: 5959; SEQ ID NO: 5960; SEQ ID NO: 5961; SEQ ID NO: 5962; SEQ ID NO: 5963; SEQ ID NO: 5964; SEQ ID NO: 5965; SEQ ID NO: 5966; SEQ ID NO: 5967; SEQ ID NO: 5968; SEQ ID NO: 5969; SEQ ID NO: 5970; SEQ ID NO: 5971; SEQ ID NO: 5972; SEQ ID NO: 5973; SEQ ID NO: 5974; SEQ ID NO: 5975; SEQ ID NO: 5976; SEQ ID NO: 5977; SEQ ID NO: 5978; SEQ ID NO: 5979; SEQ ID NO: 5980; SEQ ID NO: 5981; SEQ ID NO: 5982; SEQ ID NO: 5983; SEQ ID NO: 5984; SEQ ID NO: 5985; SEQ ID NO: 5986; SEQ ID NO: 5987; SEQ ID NO: 5988; SEQ ID NO: 5989; SEQ ID NO: 5990; SEQ ID NO: 5991; SEQ ID NO: 5992; SEQ ID NO: 5993; SEQ ID NO: 5994; SEQ ID NO: 5995; SEQ ID NO: 5996; SEQ ID NO: 5997; SEQ ID NO: 5998; SEQ ID NO: 5999; SEQ ID NO: 6000; SEQ ID NO: 6001; SEQ ID NO: 6002; SEQ ID NO: 6003; SEQ ID NO: 6004; SEQ ID NO: 6005; SEQ ID NO: 6006; SEQ ID NO: 6007; SEQ ID NO: 6008; SEQ ID NO: 6009; SEQ ID NO: 6010; SEQ ID NO: 6011; SEQ ID NO: 6012; SEQ ID NO: 6013; SEQ ID NO: 6014; SEQ ID NO: 6015; SEQ ID NO: 6016; SEQ ID NO: 6017; SEQ ID NO: 6018; SEQ ID NO: 6019; SEQ ID NO: 6020; SEQ ID NO: 6021; SEQ ID NO: 6022; SEQ ID NO: 6023; SEQ ID NO: 6024; SEQ ID NO: 6025; SEQ ID NO: 6026; SEQ ID NO: 6027; SEQ ID NO: 6028; SEQ ID NO: 6029; SEQ ID NO: 6030; SEQ ID NO: 6031; SEQ ID NO: 6032; SEQ ID NO: 6033; SEQ ID NO: 6034; SEQ ID NO: 6035; SEQ ID NO: 6036; SEQ ID NO: 6037; SEQ ID NO: 6038; SEQ ID NO: 6039; SEQ ID NO: 6040; SEQ ID NO: 6041; SEQ ID NO: 6042; SEQ ID NO: 6043; SEQ ID NO: 6044; SEQ ID NO: 6045; SEQ ID NO: 6046; SEQ TD NO: 6047; SEQ ID NO: 6048; SEQ ID NO: 6049; SEQ ID NO: 6050; SEQ ID NO: 6051; SEQ ID NO: 6052; SEQ ID NO: 6053; SEQ ID NO: 6054; SEQ ID NO: 6055; SEQ ID NO: 6056; SEQ ID NO: 6057; SEQ ID NO: 6058; SEQ ID NO: 6059; SEQ ID NO: 6060; SEQ ID NO: 6061; SEQ ID NO: 6062; SEQ ID NO: 6063; SEQ ID NO: 6064; SEQ ID NO: 6065; SEQ ID NO: 6066; SEQ ID NO: 6067; SEQ ID NO: 6068; SEQ ID NO: 6069; SEQ ID NO: 6070; SEQ ID NO: 6071; SEQ ID NO: 6072; SEQ ID NO: 6073; SEQ ID NO: 6074; SEQ ID NO: 6075; SEQ ID NO: 6076; SEQ ID NO: 6077; SEQ ID NO: 6078; SEQ ID NO: 6079; SEQ ID NO: 6080; SEQ ID NO: 6081; SEQ ID NO: 6082; SEQ ID NO: 6083; SEQ ID NO: 6084; SEQ ID NO: 6085; SEQ ID NO: 6086; SEQ ID NO: 6087; SEQ ID NO: 6088; SEQ ID NO: 6089; SEQ ID NO: 6090; SEQ ID NO: 6091; SEQ ID NO: 6092; SEQ ID NO: 6093; SEQ ID NO: 6094; SEQ ID NO: 6095; SEQ ID NO: 6096; SEQ ID NO: 6097; SEQ ID NO: 6098; SEQ ID NO: 6099; SEQ ID NO: 6100; SEQ ID NO: 6101; SEQ ID NO: 6102; SEQ ID NO: 6103; SEQ ID NO: 6104; SEQ ID NO: 6105; SEQ ID NO: 6106; SEQ ID NO: 6107; SEQ ID NO: 6108; SEQ ID NO: 6109; SEQ ID NO: 6110; SEQ ID NO: 6111; SEQ ID NO: 6112; SEQ ID NO: 6113; SEQ ID NO: 6114; SEQ ID NO: 6115; SEQ ID NO: 6116; SEQ ID NO: 6117; SEQ ID NO: 6118; SEQ ID NO: 6119; SEQ ID NO: 6120; SEQ ID NO: 6121; SEQ ID NO: 6122; SEQ ID NO: 6123; SEQ ID NO: 6124; SEQ ID NO: 6125; SEQ ID NO: 6126; SEQ ID NO: 6127; SEQ ID NO: 6128; SEQ ID NO: 6129; SEQ ID NO: 6130; SEQ ID NO: 6131; SEQ ID NO: 6132; SEQ ID NO: 6133; SEQ ID NO: 6134; SEQ ID NO: 6135; SEQ ID NO: 6136; SEQ ID NO: 6137; SEQ ID NO: 6138; SEQ ID NO: 6139; SEQ ID NO: 6140; SEQ ID NO: 6141; SEQ ID NO: 6142; SEQ ID NO: 6143; SEQ ID NO: 6144; SEQ ID NO: 6145; SEQ ID NO: 6146; SEQ ID NO: 6147; SEQ ID NO: 6148; SEQ ID NO: 6149; SEQ ID NO: 6150; SEQ ID NO: 6151; SEQ ID NO: 6152; SEQ ID NO: 6153; SEQ ID NO: 6154; SEQ ID NO: 6155; SEQ ID NO: 6156; SEQ ID NO: 6157; SEQ ID NO: 6158; SEQ ID NO: 6159; SEQ ID NO: 6160; SEQ ID NO: 6161; SEQ ID NO: 6162; SEQ ID NO: 6163; SEQ ID NO: 6164; SEQ ID NO: 6165; SEQ ID NO: 6166; SEQ ID NO: 6167; SEQ ID NO: 6168; SEQ ID NO: 6169; SEQ ID NO: 6170; SEQ ID NO: 6171; SEQ ID NO: 6172; SEQ ID NO: 6173; SEQ ID NO: 6174; SEQ ID NO: 6175; SEQ ID NO: 6176; SEQ ID NO: 6177; SEQ ID NO: 6178; SEQ ID NO: 6179; SEQ ID NO: 6180; SEQ ID NO: 6181; SEQ ID NO: 6182; SEQ ID NO: 6183; SEQ ID NO: 6184; SEQ ID NO: 6185; SEQ ID NO: 6186; SEQ ID NO: 6187; SEQ ID NO: 6188; SEQ ID NO: 6189; SEQ ID NO: 6190; SEQ ID NO: 6191; SEQ ID NO: 6192; SEQ ID NO: 6193; SEQ ID NO: 6194; SEQ ID NO: 6195; SEQ ID NO: 6196; SEQ ID NO: 6197; SEQ ID NO: 6198; SEQ ID NO: 6199; SEQ ID NO: 6200; SEQ ID NO: 6201; SEQ ID NO: 6202; SEQ ID NO: 6203; SEQ ID NO: 6204; SEQ ID NO: 6205; SEQ ID NO: 6206; SEQ ID NO: 6207; SEQ ID NO: 6208; SEQ ID NO: 6209; SEQ ID NO: 6210; SEQ ID NO: 6211; SEQ ID NO: 6212; SEQ ID NO: 6213; SEQ ID NO: 6214; SEQ ID NO: 6215; SEQ ID NO: 6216; SEQ ID NO: 6217; SEQ ID NO: 6218; SEQ ID NO: 6219; SEQ ID NO: 6220; SEQ ID NO: 6221; SEQ ID NO: 6222; SEQ ID NO: 6223; SEQ ID NO: 6224; SEQ ID NO: 6225; SEQ ID NO: 6226; SEQ ID NO: 6227; SEQ ID NO: 6228; SEQ ID NO: 6229; SEQ ID NO: 6230; SEQ ID NO: 6231; SEQ ID NO: 6232; SEQ ID NO: 6233; SEQ ID NO: 6234; SEQ ID NO: 6235; SEQ ID NO: 6236; SEQ ID NO: 6237; SEQ ID NO: 6238; SEQ ID NO: 6239; SEQ ID NO: 6240; SEQ ID NO: 6241; SEQ ID NO: 6242; SEQ ID NO: 6243; SEQ ID NO: 6244; SEQ ID NO: 6245; SEQ ID NO: 6246; SEQ ID NO: 6247; SEQ ID NO: 6248; SEQ ID NO: 6249; SEQ ID NO: 6250; SEQ ID NO: 6251; SEQ ID NO: 6252; SEQ ID NO: 6253; SEQ ID NO: 6254; SEQ ID NO: 6255; SEQ ID NO: 6256; SEQ ID NO: 6257; SEQ ID NO: 6258; SEQ ID NO: 6259; SEQ ID NO: 6260; SEQ ID NO: 6261; SEQ ID NO: 6262; SEQ ID NO: 6263; SEQ ID NO: 6264; SEQ ID NO: 6265; SEQ ID NO: 6266; SEQ ID NO: 6267; SEQ ID NO: 6268; SEQ ID NO: 6269; SEQ ID NO: 6270; SEQ ID NO: 6271; SEQ ID NO: 6272; SEQ ID NO: 6273; SEQ ID NO: 6274; SEQ ID NO: 6275; SEQ ID NO: 6276; SEQ ID NO: 9649; SEQ ID NO: 9650; SEQ ID NO: 9651; SEQ ID NO: 9652; SEQ ID NO: 9653; SEQ ID NO: 9654; SEQ ID NO: 9655; SEQ ID NO: 9656; SEQ ID NO: 9657; SEQ ID NO: 9658; SEQ ID NO: 9659; SEQ ID NO: 9660; SEQ ID NO: 9661; SEQ ID NO: 9662; SEQ ID NO: 9663; SEQ ID NO: 9664; SEQ ID NO: 9665; SEQ ID NO: 9666; SEQ ID NO: 9667; SEQ ID NO: 9668; SEQ ID NO: 9669; SEQ ID NO: 9670; SEQ ID NO: 9671; SEQ ID NO: 9672; SEQ ID NO: 9673; SEQ ID NO: 9674; SEQ ID NO: 9675; SEQ ID NO: 9676; SEQ ID NO: 9677; SEQ ID NO: 9678; SEQ ID NO: 9679; SEQ ID NO: 9680; SEQ ID NO: 9681; SEQ ID NO: 9682; SEQ ID NO: 9683; SEQ ID NO: 9684; SEQ ID NO: 9685; SEQ ID NO: 9686; SEQ ID NO: 9687; SEQ ID NO: 9688; SEQ ID NO: 9689; SEQ ID NO: 9690; SEQ ID NO: 9691; SEQ ID NO: 9692; SEQ ID NO: 9693; SEQ ID NO: 9694; SEQ ID NO: 9695; SEQ ID NO: 9696; SEQ ID NO: 9697; SEQ ID NO: 9698; SEQ ID NO: 9699; SEQ ID NO: 9700; SEQ ID NO: 9701; SEQ ID NO: 9702 and SEQ ID NO: 9703.

In another embodiment, the H. pylori cell envelope polypeptide or a fragment thereof is an H. pylori inner membrane polypeptide or a fragment thereof involved in transport having an amino acid sequence selected from the group consisting of SEQ ID NO: 5273; SEQ ID NO: 5274; SEQ ID NO: 5275; SEQ ID NO: 5276; SEQ ID NO: 5277; SEQ ID NO: 5278; SEQ ID NO: 5279; SEQ ID NO: 5280; SEQ ID NO: 5281; SEQ ID NO: 5282; SEQ ID NO: 5283; SEQ ID NO: 5284; SEQ ID NO: 5285; SEQ ID NO: 5286; SEQ ID NO: 5287; SEQ ID NO: 5288; SEQ ID NO: 5289; SEQ ID NO: 5290; SEQ ID NO: 5291; SEQ ID NO: 5292; SEQ ID NO: 5293; SEQ ID NO: 5294; SEQ ID NO: 5295; SEQ ID NO: 5296; SEQ ID NO: 5297; SEQ ID NO: 5298; SEQ ID NO: 5299; SEQ ID NO: 5300; SEQ ID NO: 5301; SEQ ID NO: 5302; SEQ ID NO: 5303; SEQ ID NO: 5304; SEQ ID NO: 5305; SEQ ID NO: 5306; SEQ ID NO: 5307; SEQ ID NO: 5308; SEQ ID NO: 5309; SEQ ID NO: 5310; SEQ ID NO: 5311; SEQ ID NO: 5312; SEQ ID NO: 5313; SEQ ID NO: 5314; SEQ ID NO: 5315; SEQ ID NO: 5316; SEQ ID NO: 5317; SEQ ID NO: 5318; SEQ ID NO: 5319; SEQ ID NO: 5320; SEQ ID NO: 5321; SEQ ID NO: 5322; SEQ ID NO: 5323; SEQ ID NO: 5324; SEQ ID NO: 5325; SEQ ID NO: 5326; SEQ ID NO: 5327; SEQ ID NO: 5328; SEQ ID NO: 5329; SEQ ID NO: 5330; SEQ ID NO:5331; SEQ ID NO: 5332; SEQ ID NO: 5333; SEQ ID NO: 5334; SEQ ID NO: 5335; SEQ ID NO: 5336; SEQ ID NO: 5337; SEQ ID NO: 5338; SEQ ID NO: 5339; SEQ ID NO: 5340; SEQ ID NO: 5341; SEQ ID NO: 5342; SEQ ID NO: 5343; SEQ ID NO: 5344; SEQ ID NO: 5345; SEQ ID NO: 5346; SEQ ID NO: 5347; SEQ ID NO: 5348; SEQ ID NO: 5349; SEQ ID NO: 5350; SEQ ID NO: 5351; SEQ ID NO: 5352; SEQ ID NO: 5353; SEQ ID NO: 5354; SEQ ID NO: 5355; SEQ ID NO: 5356; SEQ ID NO: 5357; SEQ ID NO: 5358; SEQ ID NO: 5359; SEQ ID NO: 5360; SEQ ID NO: 5361; SEQ ID NO: 5362; SEQ ID NO: 5363; SEQ ID NO: 5364; SEQ ID NO: 5365; SEQ ID NO: 5366; SEQ ID NO: 5367; SEQ ID NO: 5368; SEQ ID NO: 5369; SEQ ID NO: 5370; SEQ ID NO: 5371; SEQ ID NO: 5372; SEQ ID NO: 5373; SEQ ID NO: 5374; SEQ ID NO: 5375; SEQ ID NO: 5376; SEQ ID NO: 5377; SEQ ID NO: 5378; SEQ ID NO: 5379; SEQ ID NO: 5380; SEQ ID NO: 5381; SEQ ID NO: 5382; SEQ ID NO: 5383; SEQ ID NO: 5384; SEQ ID NO: 5385; SEQ ID NO: 5386; SEQ ID NO: 5387; SEQ ID NO: 5388; SEQ ID NO: 5389; SEQ ID NO: 5390; SEQ ID NO: 5391; SEQ ID NO: 5392; SEQ ID NO: 5393; SEQ ID NO: 5394; SEQ ID NO: 5395; SEQ ID NO: 5396; SEQ ID NO: 5397; SEQ ID NO: 5398; SEQ ID NO: 5399; SEQ ID NO: 5400; SEQ ID NO: 5401; SEQ ID NO: 5402; SEQ ID NO: 5403; SEQ ID NO: 5404; SEQ ID NO: 5405; SEQ ID NO: 5406; SEQ ID NO: 5407; SEQ ID NO: 5408; SEQ ID NO: 5409; SEQ ID NO: 5410; SEQ ID NO: 5411; SEQ ID NO: 5412; SEQ ID NO: 5413; SEQ ID NO: 5414; SEQ ID NO: 5415; SEQ ID NO: 5416; SEQ ID NO: 5417; SEQ ID NO: 5418; SEQ ID NO: 5419; SEQ ID NO: 5420; SEQ ID NO: 5421; SEQ ID NO: 5422; SEQ ID NO: 5423; SEQ ID NO: 5424; SEQ ID NO: 5425; SEQ ID NO: 5426; SEQ ID NO: 5427; SEQ ID NO: 5428; SEQ ID NO:5429; SEQ ID NO: 5430; SEQ ID NO:5431; SEQ ID NO: 5432; SEQ ID NO: 5433; SEQ ID NO:5434; SEQ ID NO: 5435; SEQ ID NO: 5436; SEQ ID NO: 5437; SEQ ID NO: 5438; SEQ ID NO: 5439; SEQ ID NO: 5440; SEQ ID NO: 5441; SEQ ID NO: 5442; SEQ ID NO: 5443; SEQ ID NO: 5444; SEQ ID NO: 5564; SEQ ID NO: 9649; SEQ ID NO: 9650; SEQ ID NO: 9651; SEQ ID NO: 9652; SEQ ID NO: 9653; SEQ ID NO: 9654; SEQ ID NO: 9655; SEQ ID NO: 9656; SEQ ID NO: 9657; SEQ ID NO: 9658; SEQ ID NO: 9659; SEQ ID NO: 9660; SEQ ID NO: 9661; SEQ ID NO: 9662; SEQ ID NO: 9663; SEQ ID NO: 9664; SEQ ID NO: 9665 and SEQ ID NO: 9666.

In another embodiment, the H. pylori cell envelope polypeptide or a fragment thereof is an H. pylori inner membrane polypeptide or a fragment thereof involved in amino acid metabolism and transport having an amino acid sequence selected from the group consisting of SEQ ID NO: 5445; SEQ ID NO: 5446; SEQ ID NO: 5447; SEQ ID NO: 5448; SEQ ID NO: 5449; SEQ ID NO: 5450; SEQ ID NO: 5451; SEQ ID NO: 5452; SEQ ID NO: 5453; SEQ ID NO: 5454; SEQ ID NO: 5455; SEQ ID NO: 5456; SEQ ID NO: 5457; SEQ ID NO: 5458; SEQ ID NO: 5459; SEQ ID NO: 5460; SEQ ID NO: 5461; SEQ ID NO: 5462; SEQ ID NO: 5463; SEQ ID NO: 5464; SEQ ID NO: 5465; SEQ ID NO: 5466; SEQ ID NO: 5467; SEQ ID NO: 5468; SEQ ID NO: 5469; SEQ ID NO: 5470; SEQ ID NO: 5471; SEQ ID NO: 5472; SEQ ID NO: 5473; SEQ ID NO: 5474; SEQ ID NO: 5475; SEQ ID NO: 5476; SEQ ID NO: 5477; SEQ ID NO: 5478; SEQ ID NO: 5479; SEQ ID NO: 5480; SEQ ID NO: 5481; SEQ ID NO: 5482; SEQ ID NO: 9659 and SEQ ID NO: 9660.

In another embodiment, the H. pylori cell envelope polypeptide or a fragment thereof is an H. pylori inner membrane polypeptide or a fragment thereof involved in nucleotide, lipid, or cofactor metabolism and transport having an amino acid sequence selected from the group consisting of SEQ ID NO: 5483; SEQ ID NO: 5484; SEQ ID NO: 5485; SEQ ID NO: 5486; SEQ ID NO: 5487; SEQ ID NO: 5488; SEQ ID NO: 5489; SEQ ID NO: 5490; SEQ ID NO: 5491; SEQ ID NO: 5492; SEQ ID NO: 5493; SEQ ID NO: 5494; SEQ ID NO: 5495; SEQ ID NO: 5496; SEQ ID NO: 5497; SEQ ID NO: 5498; SEQ ID NO: 5499; SEQ ID NO: 5500; SEQ ID NO: 5501; SEQ ID NO: 5502; SEQ ID NO: 5503; SEQ ID NO: 5504; SEQ ID NO: 5505; SEQ ID NO: 5506; SEQ ID NO: 5507; SEQ ID NO: 5508; SEQ ID NO: 5509; SEQ ID NO: 5510; SEQ ID NO: 5511; SEQ ID NO: 5512; SEQ ID NO: 5513; SEQ ID NO: 5514; SEQ ID NO: 5515; SEQ ID NO: 5516; SEQ ID NO: 5517; SEQ ID NO: 5518; SEQ ID NO: 5519; SEQ ID NO: 5520; SEQ ID NO: 5521; SEQ ID NO: 5522; SEQ ID NO: 5523; SEQ ID NO: 5524; SEQ ID NO: 5525; SEQ ID NO: 5526 and SEQ ID NO: 9661.

In another embodiment, the H. pylori cell envelope polypeptide or a fragment thereof is an H. pylori inner membrane polypeptide or a fragment thereof involved in inorganic ion transport having an amino acid sequence selected from the group consisting of SEQ ID NO: 5527; SEQ ID NO: 5528; SEQ ID NO: 5529; SEQ ID NO: 5530; SEQ ID NO: 5531; SEQ ID NO: 5532; SEQ ID NO: 5533; SEQ ID NO: 5534; SEQ ID NO: 5535; SEQ ID NO: 5536; SEQ ID NO: 5537; SEQ ID NO: 5538; SEQ ID NO: 5539; SEQ ID NO: 5540; SEQ ID NO: 5541; SEQ ID NO: 5542; SEQ ID NO: 5543; SEQ ID NO: 5544; SEQ ID NO: 5545; SEQ ID NO: 5546; SEQ ID NO: 5547; SEQ ID NO: 5548; SEQ ID NO: 5549; SEQ ID NO: 5550; SEQ ID NO: 5551; SEQ ID NO: 5552; SEQ ID NO: 5553; SEQ ID NO: 5554; SEQ ID NO: 5555; SEQ ID NO: 5556; SEQ ID NO: 5557; SEQ ID NO: 5558; SEQ ID NO: 5559; SEQ ID NO: 5560; SEQ ID NO: 5561; SEQ ID NO: 9662; SEQ ID NO: 9663; SEQ ID NO: 9664; SEQ ID NO: 9665 and SEQ ID NO: 9666.

In another embodiment, the H. pylori cell envelope polypeptide or a fragment thereof is an H. pylori inner membrane polypeptide or a fragment thereof involved in carbohydrate metabolism and transport having an amino acid sequence selected from the group consisting of SEQ ID NO: 5562; SEQ ID NO: 5563 and SEQ ID NO: 5564.

In another embodiment, the H. pylori cell envelope polypeptide or a fragment thereof is an H. pylori inner membrane polypeptide or a fragment thereof involved in outer membrane and cell wall formation having an amino acid sequence selected from the group consisting of SEQ ID NO: 5565; SEQ ID NO: 5566; SEQ ID NO: 5567; SEQ ID NO: 5568; SEQ ID NO: 5569; SEQ ID NO: 5570; SEQ ID NO: 5571; SEQ ID NO: 5572; SEQ ID NO: 5573; SEQ ID NO: 5574; SEQ ID NO: 5575; SEQ ID NO: 5576; SEQ ID NO: 5577; SEQ ID NO: 5578; SEQ ID NO: 5579; SEQ ID NO: 5580; SEQ ID NO: 5581; SEQ ID NO: 5582; SEQ ID NO: 5583; SEQ ID NO: 5584; SEQ ID NO: 5585; SEQ ID NO: 5586; SEQ ID NO: 5587; SEQ ID NO: 5588; SEQ ID NO: 5589; SEQ ID NO: 5590; SEQ ID NO: 5591; SEQ ID NO: 5592; SEQ ID NO: 5593; SEQ ID NO: 5594; SEQ ID NO: 5595; SEQ ID NO: 5596; SEQ ID NO: 5597; SEQ ID NO: 5598; SEQ ID NO: 5599; SEQ ID NO: 5600; SEQ ID NO: 5601; SEQ ID NO: 5602; SEQ ID NO: 5603; SEQ ID NO: 5604; SEQ ID NO: 5605; SEQ ID NO: 5606; SEQ ID NO: 5607; SEQ ID NO: 5608; SEQ ID NO: 5609; SEQ ID NO: 5610; SEQ ID NO: 9667; SEQ ID NO: 9668; SEQ ID NO: 9669; SEQ ID NO: 9670; SEQ ID NO: 9671 and SEQ ID NO: 9672.

In another embodiment, the H. pylori cell envelope polypeptide or a fragment thereof is an H. pylori inner membrane polypeptide or a fragment thereof involved in energy conversion having an amino acid sequence selected from the group consisting of SEQ ID NO: 5611; SEQ ID NO: 5612; SEQ ID NO: 5613; SEQ ID NO: 5614; SEQ ID NO: 5615; SEQ ID NO: 5616; SEQ ID NO: 5617; SEQ ID NO: 5618; SEQ ID NO: 5619; SEQ ID NO: 5620; SEQ ID NO: 5621; SEQ ID NO: 5622; SEQ ID NO: 5623; SEQ ID NO: 5624; SEQ ID NO: 5625; SEQ ID NO: 5626; SEQ ID NO: 5627; SEQ ID NO: 5628; SEQ ID NO: 5629; SEQ ID NO: 5630; SEQ ID NO: 5631; SEQ ID NO: 5632; SEQ ID NO: 5633; SEQ ID NO: 5634; SEQ ID NO: 5635; SEQ ID NO: 5636; SEQ ID NO: 5637; SEQ ID NO: 5638; SEQ ID NO: 5639; SEQ ID NO: 5640; SEQ ID NO: 5641; SEQ ID NO: 5642; SEQ ID NO: 5643; SEQ ID NO: 5644; SEQ ID NO: 5645; SEQ ID NO: 5646; SEQ ID NO: 5647; SEQ ID NO: 5648; SEQ ID NO: 5649; SEQ ID NO: 5650; SEQ ID NO: 5651; SEQ ID NO: 5652; SEQ ID NO:5653; SEQ ID NO: 5654; SEQ ID NO: 5655; SEQ ID NO: 5656; SEQ ID NO: 5657; SEQ ID NO: 5658; SEQ ID NO: 5659; SEQ ID NO: 5660; SEQ ID NO: 5661; SEQ ID NO: 5662; SEQ ID NO: 5663; SEQ ID NO: 5664; SEQ ID NO: 5665; SEQ ID NO: 5666; SEQ ID NO: 5667; SEQ ID NO: 5668; SEQ ID NO: 5669; SEQ ID NO: 5670; SEQ ID NO: 5671; SEQ ID NO: 5672; SEQ ID NO: 5673; SEQ ID NO: 5674; StQ ID NO: 5675; SEQ ID NO: 5676; SEQ ID NO: 5677; SEQ ID NO: 5678; SEQ ID NO: 5679; SEQ ID NO: 5680; SEQ ID NO: 5681; SEQ ID NO:5682; SEQ ID NO: 5683; SEQ ID NO: 5684; SEQ ID NO: 5685; SEQ ID NO: 5686; SEQ ID NO: 5687; SEQ ID NO: 5688; SEQ ID NO: 5689; SEQ ID NO: 5690; SEQ ID NO: 5691; SEQ ID NO: 5692; SEQ ID NO: 5693; SEQ ID NO: 5694; SEQ ID NO: 5695; SEQ ID NO: 5696; SEQ ID NO: 5697; SEQ ID NO: 5698; SEQ ID NO: 5699; SEQ ID NO: 5700; SEQ ID NO: 5701; SEQ ID NO: 5702; SEQ ID NO: 5703; SEQ ID NO: 5704; SEQ ID NO: 5705; SEQ ID NO: 5706; SEQ ID NO: 5707; SEQ ID NO: 5708; SEQ ID NO: 5709; SEQ ID NO: 5710; SEQ ID NO: 5711; SEQ ID NO: 5712; SEQ ID NO: 5713; SEQ ID NO: 5714; SEQ ID NO: 5715; SEQ ID NO: 5716; SEQ ID NO: 5717; SEQ ID NO: 5718; SEQ ID NO: 5719; SEQ ID NO: 5720; SEQ ID NO: 5721; SEQ ID NO: 5722; SEQ ID NO: 5723; SEQ ID NO: 5724; SEQ ID NO: 5725; SEQ ID NO: 5726; SEQ ID NO: 5727; SEQ ID NO: 5728; SEQ ID NO: 5729; SEQ ID NO: 5730; SEQ ID NO: 5731; SEQ ID NO: 5732; SEQ ID NO: 5733; SEQ ID NO: 5734; SEQ ID NO: 5735; SEQ ID NO: 5736; SEQ ID NO: 5737; SEQ ID NO: 5738; SEQ ID NO: 5739; SEQ ID NO: 5740; SEQ ID NO: 5741; SEQ ID NO: 5742; SEQ ID NO: 5743; SEQ ID NO: 5744; SEQ ID NO: 5745; SEQ ID NO: 5746; SEQ ID NO: 5747; SEQ ID NO: 5748; SEQ ID NO: 5749; SEQ ID NO: 5750; SEQ ID NO: 5751; SEQ ID NO: 5752; SEQ ID NO: 5753; SEQ ID NO: 5754; SEQ ID NO: 5755; SEQ ID NO: 5756; SEQ ID NO: 5757; SEQ ID NO: 9673; SEQ ID NO: 9674; SEQ ID NO: 9675; SEQ ID NO: 9676; SEQ ID NO: 9677.

In another embodiment, the H. pylori cell envelope polypeptide or a fragment thereof is an H. pylori inner membrane polypeptide or a fragment thereof involved in secretion and adhesion having an amino acid sequence selected from the group consisting of SEQ ID NO: 5758; SEQ ID NO: 5759; SEQ ID NO: 5760; SEQ ID NO: 5761; SEQ ID NO: 5762; SEQ ID NO: 5763; SEQ ID NO: 5764; SEQ ID NO: 5765; SEQ ID NO: 5766; SEQ ID NO: 5767; SEQ ID NO: 5768; SEQ ID NO: 5769; SEQ ID NO: 9678.

In another embodiment, the H. pylori cell envelope polypeptide or a fragment thereof is an H. pylori inner membrane polypeptide or a fragment thereof involved in regulation having an amino acid sequence selected from the group consisting of SEQ ID NO: 5770; SEQ ID NO: 5771; SEQ ID NO: 5772; SEQ ID NO: 5773; SEQ ID NO: 5774; SEQ ID NO: 5775; SEQ ID NO: 5776; SEQ ID NO: 5777; SEQ ID NO: 5778; SEQ ID NO: 5779; SEQ ID NO: 5780; SEQ ID NO: 5781; SEQ ID NO: 5782; SEQ ID NO: 5783; SEQ ID NO: 5784; SEQ ID NO: 5785; SEQ ID NO: 5786; SEQ ID NO: 5787; SEQ ID NO: 5788; SEQ ID NO: 5789.

In another embodiment, the H. pylori cell envelope polypeptide or a fragment thereof is an H. pylori inner membrane chaperone polypeptide or a fragment thereof having an amino acid sequence selected from the group consisting of SEQ ID NO: 5790; SEQ ID NO: 5791; SEQ ID NO: 5792; SEQ ID NO: 5793 and SEQ ID NO: 9679.

In another embodiment, the H. pylori cell envelope polypeptide or a fragment thereof is an H. pylori inner membrane polypeptide or a fragment thereof involved in cell division having an amino acid sequence selected from the group consisting of SEQ ID NO: 5794; SEQ ID NO: 5795; SEQ ID NO: 5796; SEQ ID NO: 5797; SEQ ID NO: 5798; SEQ ID NO: 5799; SEQ ID NO: 5800; SEQ ID NO: 5801; SEQ ID NO: 5802; SEQ ID NO: 5803; SEQ ID NO: 5804; SEQ ID NO: 5805; SEQ ID NO: 5806; SEQ ID NO: 5807; SEQ ID NO: 5808; SEQ ID NO: 5809; SEQ ID NO: 5810; SEQ ID NO: 5811; SEQ ID NO: 5812; SEQ ID NO: 5813; SEQ ID NO: 5814; SEQ ID NO: 9680 and SEQ ID NO: 9681.

In another embodiment, the H. pylori cell envelope polypeptide or a fragment thereof is an H. pylori inner membrane polypeptide or a fragment thereof involved in motility having an amino acid sequence selected from the group consisting of SEQ ID NO: 5815 and SEQ ID NO: 5816.

Particularly preferred is an isolated H. pylori cell envelope polypeptide or a fragment thereof, wherein the polypeptide is encoded by a nucleic acid sequence selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO:11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO: 117; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; SEQ ID NO: 186; SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; SEQ ID NO: 211; SEQ ID NO: 212; SEQ ID NO: 213; SEQ ID NO: 214; SEQ ID NO: 215; SEQ ID NO: 216; SEQ ID NO: 217; SEQ ID NO: 218; SEQ ID NO: 219; SEQ ID NO: 220; SEQ ID NO: 221; SEQ ID NO: 222; SEQ ID NO: 223; SEQ ID NO: 224; SEQ ID NO: 225; SEQ ID NO: 226; SEQ ID NO: 227; SEQ ID NO: 228; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 236; SEQ ID NO: 237; SEQ ID NO: 238; SEQ ID NO: 239; SEQ ID NO: 240; SEQ ID NO: 241; SEQ ID NO: 242; SEQ ID NO: 243; SEQ ID NO: 244; SEQ ID NO: 245; SEQ ID NO: 246; SEQ ID NO: 247; SEQ ID NO: 248; SEQ ID NO: 249; SEQ ID NO: 250; SEQ ID NO: 251; SEQ ID NO: 252; SEQ ID NO: 253; SEQ ID NO: 254; SEQ ID NO: 255; SEQ ID NO: 256; SEQ ID NO: 257; SEQ ID NO: 258; SEQ ID NO: 259; SEQ ID NO: 260; SEQ ID NO: 261; SEQ ID NO: 262; SEQ ID NO: 263; SEQ ID NO: 264; SEQ ID NO: 265; SEQ ID NO: 266; SEQ ID NO: 267; SEQ ID NO: 268; SEQ ID NO: 269; SEQ ID NO: 270; SEQ ID NO: 271; SEQ ID NO: 272; SEQ ID NO: 273; SEQ ID NO: 274; SEQ ID NO: 275; SEQ ID NO: 276; SEQ ID NO: 277; SEQ ID NO: 278; SEQ ID NO: 279; SEQ ID NO: 280; SEQ ID NO: 281; SEQ ID NO: 282; SEQ ID NO: 283; SEQ ID NO: 284; SEQ ID NO: 285; SEQ ID NO: 286; SEQ ID NO: 287; SEQ ID NO: 288; SEQ ID NO: 289; SEQ ID NO: 290; SEQ ID NO: 291; SEQ ID NO: 292; SEQ ID NO: 293; SEQ ID NO: 294; SEQ ID NO: 295; SEQ ID NO: 296; SEQ ID NO: 297; SEQ ID NO: 298; SEQ ID NO: 299; SEQ ID NO: 300; SEQ ID NO: 301; SEQ ID NO: 302; SEQ ID NO: 303; SEQ ID NO: 304; SEQ ID NO: 305; SEQ ID NO: 306; SEQ ID NO: 307; SEQ ID NO: 308; SEQ ID NO: 309; SEQ ID NO: 310; SEQ ID NO: 311; SEQ ID NO: 312; SEQ ID NO: 313; SEQ ID NO: 314; SEQ ID NO: 315; SEQ ID NO: 316; SEQ ID NO: 317; SEQ ID NO: 318; SEQ ID NO: 319; SEQ ID NO: 320; SEQ ID NO: 321; SEQ ID NO: 322; SEQ ID NO: 323; SEQ ID NO: 324; SEQ ID NO: 325; SEQ ID NO: 326; SEQ ID NO: 327; SEQ ID NO: 328; SEQ ID NO: 329; SEQ ID NO: 330; SEQ ID NO: 331; SEQ ID NO: 332; SEQ ID NO: 333; SEQ ID NO: 334; SEQ ID NO: 335; SEQ ID NO: 336; SEQ ID NO: 337; SEQ ID NO: 338; SEQ ID NO: 339; SEQ ID NO: 340; SEQ ID NO: 341; SEQ ID NO: 342; SEQ ID NO: 343; SEQ ID NO: 344; SEQ ID NO: 345; SEQ ID NO: 346; SEQ ID NO: 347; SEQ ID NO: 348; SEQ ID NO: 349; SEQ ID NO: 350; SEQ ID NO: 351; SEQ ID NO: 352; SEQ ID NO: 353; SEQ ID NO: 354; SEQ ID NO: 355; SEQ ID NO: 356; SEQ ID NO: 357; SEQ ID NO: 358; SEQ ID NO: 359; SEQ ID NO: 360; SEQ ID NO: 361; SEQ ID NO: 362; SEQ ID NO: 363; SEQ ID NO: 364; SEQ ID NO: 365; SEQ ID NO: 366; SEQ ID NO: 367; SEQ ID NO: 368; SEQ ID NO: 369; SEQ ID NO: 370; SEQ ID NO: 371; SEQ ID NO: 372; SEQ ID NO: 373; SEQ ID NO: 374; SEQ ID NO: 375; SEQ ID NO: 376; SEQ ID NO: 377; SEQ ID NO: 378; SEQ ID NO: 379; SEQ ID NO: 380; SEQ ID NO: 381; SEQ ID NO: 382; SEQ ID NO: 383; SEQ ID NO: 384; SEQ ID NO: 385; SEQ ID NO: 386; SEQ ID NO: 387; SEQ ID NO: 388; SEQ ID NO: 389; SEQ ID NO: 390; SEQ ID NO: 391; SEQ ID NO: 392; SEQ ID NO: 393; SEQ ID NO: 394; SEQ ID NO: 395; SEQ ID NO: 396; SEQ ID NO: 397; SEQ ID NO: 398; SEQ ID NO: 399; SEQ ID NO: 400; SEQ ID NO: 401; SEQ ID NO: 402; SEQ ID NO: 403; SEQ ID NO: 404; SEQ ID NO: 405; SEQ ID NO: 406; SEQ ID NO: 407; SEQ ID NO: 408; SEQ ID NO: 409; SEQ ID NO: 410; SEQ ID NO: 411; SEQ ID NO: 412; SEQ ID NO: 413; SEQ ID NO: 414; SEQ ID NO: 415; SEQ ID NO: 416; SEQ ID NO: 417; SEQ ID NO: 418; SEQ ID NO: 419; SEQ ID NO: 420; SEQ ID NO: 421; SEQ ID NO: 422; SEQ ID NO: 423; SEQ ID NO: 424; SEQ ID NO: 425; SEQ ID NO: 426; SEQ ID NO: 427; SEQ ID NO: 428; SEQ ID NO: 429; SEQ ID NO: 430; SEQ ID NO: 431; SEQ ID NO: 432; SEQ ID NO: 433; SEQ ID NO: 434; SEQ ID NO: 435; SEQ ID NO: 436; SEQ ID NO: 437; SEQ ID NO: 438; SEQ ID NO: 439; SEQ ID NO: 440; SEQ ID NO: 441; SEQ ID NO: 442; SEQ ID NO: 443; SEQ ID NO: 444; SEQ ID NO: 445; SEQ ID NO: 446; SEQ ID NO: 447; SEQ ID NO: 448; SEQ ID NO: 449; SEQ ID NO: 450; SEQ ID NO: 451; SEQ ID NO: 452; SEQ ID NO: 453; SEQ ID NO: 454; SEQ ID NO: 455; SEQ ID NO: 456; SEQ ID NO: 457; SEQ ID NO: 458; SEQ ID NO: 459; SEQ ID NO: 460; SEQ ID NO: 461; SEQ ID NO: 462; SEQ ID NO: 463; SEQ ID NO: 464; SEQ ID NO: 465; SEQ ID NO: 466; SEQ ID NO: 467; SEQ ID NO: 468; SEQ ID NO: 469; SEQ ID NO: 470; SEQ ID NO: 471; SEQ ID NO: 472; SEQ ID NO: 473; SEQ ID NO: 474; SEQ ID NO: 475; SEQ ID NO: 476; SEQ ID NO: 477; SEQ ID NO: 478; SEQ ID NO: 479; SEQ ID NO: 480; SEQ ID NO: 481; SEQ ID NO: 482; SEQ ID NO: 483; SEQ ID NO: 484; SEQ ID NO: 485; SEQ ID NO: 486; SEQ ID NO: 487; SEQ ID NO: 488; SEQ ID NO: 489; SEQ ID NO: 490; SEQ ID NO: 491; SEQ ID NO: 492; SEQ ID NO: 493; SEQ ID NO: 494; SEQ ID NO: 495; SEQ ID NO: 496; SEQ ID NO: 497; SEQ ID NO: 498; SEQ ID NO: 499; SEQ ID NO: 500; SEQ ID NO: 501; SEQ ID NO: 502; SEQ ID NO: 503; SEQ ID NO: 504; SEQ ID NO: 505; SEQ ID NO: 506; SEQ ID NO: 507; SEQ ID NO: 508; SEQ ID NO: 509; SEQ ID NO: 510; SEQ ID NO: 511; SEQ ID NO: 512; SEQ ID NO: 513; SEQ ID NO: 514; SEQ ID NO: 515; SEQ ID NO: 516; SEQ ID NO: 517; SEQ ID NO: 518; SEQ ID NO: 519; SEQ ID NO: 520; SEQ ID NO: 521; SEQ ID NO: 522; SEQ ID NO: 523; SEQ ID NO: 524; SEQ ID NO: 525; SEQ ID NO: 526; SEQ ID NO: 527; SEQ ID NO: 528; SEQ ID NO: 529; SEQ ID NO: 530; SEQ ID NO: 531; SEQ ID NO: 532; SEQ ID NO: 533; SEQ ID NO: 534; SEQ ID NO: 535; SEQ ID NO: 536; SEQ ID NO: 537; SEQ ID NO: 538; SEQ ID NO: 539; SEQ ID NO: 540; SEQ ID NO: 541; SEQ ID NO: 542; SEQ ID NO: 543; SEQ ID NO: 544; SEQ ID NO: 545; SEQ ID NO: 546; SEQ ID NO: 547; SEQ ID NO: 548; SEQ ID NO: 549; SEQ ID NO: 550; SEQ ID NO: 551; SEQ ID NO: 552; SEQ ID NO: 553; SEQ ID NO: 554; SEQ ID NO: 555; SEQ ID NO: 556; SEQ ID NO: 557; SEQ ID NO: 558; SEQ ID NO: 559; SEQ ID NO: 560; SEQ ID NO: 561; SEQ ID NO: 562; SEQ ID NO: 563; SEQ ID NO: 564; SEQ ID NO: 565; SEQ ID NO: 566; SEQ ID NO: 567; SEQ ID NO: 568; SEQ ID NO: 569; SEQ ID NO: 570; SEQ ID NO: 571; SEQ ID NO: 572; SEQ ID NO: 573; SEQ ID NO: 574; SEQ ID NO: 575; SEQ ID NO: 576; SEQ ID NO: 577; SEQ ID NO: 578; SEQ ID NO: 579; SEQ ID NO: 580; SEQ ID NO: 581; SEQ ID NO: 582; SEQ ID NO: 583; SEQ ID NO: 584; SEQ ID NO: 585; SEQ ID NO: 586; SEQ ID NO: 587; SEQ ID NO: 588; SEQ ID NO: 589; SEQ ID NO: 590; SEQ ID NO: 591; SEQ ID NO: 592; SEQ ID NO: 593; SEQ ID NO: 594; SEQ ID NO: 595; SEQ ID NO: 596; SEQ ID NO: 597; SEQ ID NO: 598; SEQ ID NO: 599; SEQ ID NO: 600; SEQ ID NO: 601; SEQ ID NO: 602; SEQ ID NO: 603; SEQ ID NO: 604; SEQ ID NO: 605; SEQ ID NO: 606; SEQ ID NO: 607; SEQ ID NO: 608; SEQ ID NO: 609; SEQ ID NO: 610; SEQ ID NO: 611; SEQ ID NO: 612; SEQ ID NO: 613; SEQ ID NO: 614; SEQ ID NO: 615; SEQ ID NO: 616; SEQ ID NO: 617; SEQ ID NO: 618; SEQ ID NO: 619; SEQ ID NO: 620; SEQ ID NO: 621; SEQ ID NO: 622; SEQ ID NO: 623; SEQ ID NO-624; SEQ ID NO: 625; SEQ ID NO: 626; SEQ ID NO: 627; SEQ ID NO: 628; SEQ ID NO: 629; SEQ ID NO: 630; SEQ ID NO: 631; SEQ ID NO: 632; SEQ ID NO: 633; SEQ ID NO: 634; SEQ ID NO: 635; SEQ ID NO: 636; SEQ ID NO: 637; SEQ ID NO: 638; SEQ ID NO: 639; SEQ ID NO: 640; SEQ ID NO: 641; SEQ ID NO: 642; SEQ ID NO: 643; SEQ ID NO: 644; SEQ ID NO: 645; SEQ ID NO: 646; SEQ ID NO: 647; SEQ ID NO: 648; SEQ ID NO: 649; SEQ ID NO: 650; SEQ ID NO: 651; SEQ ID NO: 652; SEQ ID NO: 653; SEQ ID NO: 654; SEQ ID NO: 655; SEQ ID NO: 656; SEQ ID NO: 657; SEQ ID NO: 658; SEQ ID NO: 659; SEQ ID NO: 660; SEQ ID NO: 661; SEQ ID NO: 662; SEQ ID NO: 663; SEQ ID NO: 664; SEQ ID NO: 665; SEQ ID NO: 666; SEQ ID NO: 667; SEQ ID NO: 668; SEQ ID NO: 669; SEQ ID NO: 670; SEQ ID NO: 671; SEQ ID NO: 672; SEQ ID NO: 673; SEQ ID NO: 674; SEQ ID NO: 675; SEQ ID NO: 676; SEQ ID NO: 677; SEQ ID NO: 678; SEQ ID NO: 679; SEQ ID NO: 680; SEQ ID NO: 681; SEQ ID NO: 682; SEQ ID NO: 683; SEQ ID NO: 684; SEQ ID NO: 685; SEQ ID NO: 686; SEQ ID NO: 687; SEQ ID NO: 688; SEQ ID NO: 689; SEQ ID NO: 690; SEQ ID NO: 691; SEQ ID NO: 692; SEQ ID NO: 693; SEQ ID NO: 694; SEQ ID NO: 695; SEQ ID NO: 696; SEQ ID NO: 697; SEQ ID NO: 698; SEQ ID NO: 699; SEQ ID NO: 700; SEQ ID NO: 701; SEQ ID NO: 702; SEQ ID NO: 703; SEQ ID NO: 704; SEQ ID NO: 705; SEQ ID NO: 706; SEQ ID NO: 707; SEQ ID NO: 708; SEQ ID NO: 709; SEQ ID NO: 710; SEQ ID NO: 711; SEQ ID NO: 712; SEQ ID NO: 713; SEQ ID NO: 714; SEQ ID NO: 715; SEQ ID NO: 716; SEQ ID NO: 717; SEQ ID NO: 718; SEQ ID NO: 719; SEQ ID NO: 720; SEQ ID NO: 721; SEQ ID NO: 722; SEQ ID NO: 723; SEQ ID NO: 724; SEQ ID NO: 725; SEQ ID NO: 726; SEQ ID NO: 727; SEQ ID NO: 728; SEQ ID NO: 729; SEQ ID NO: 730; SEQ ID NO: 731; SEQ ID NO: 732; SEQ ID NO: 733; SEQ ID NO: 734; SEQ ID NO: 735; SEQ ID NO: 736; SEQ ID NO: 737; SEQ ID NO: 738; SEQ ID NO: 739; SEQ ID NO: 740; SEQ ID NO: 741; SEQ ID NO: 742; SEQ ID NO: 743; SEQ ID NO: 744; SEQ ID NO: 745; SEQ ID NO: 746; SEQ ID NO: 747; SEQ ID NO: 748; SEQ ID NO: 749; SEQ ID NO: 750; SEQ ID NO: 751; SEQ ID NO: 752; SEQ ID NO: 753; SEQ ID NO: 754; SEQ ID NO: 755; SEQ ID NO: 756; SEQ ID NO: 757; SEQ ID NO: 758; SEQ ID NO: 759; SEQ ID NO: 760; SEQ ID NO: 761; SEQ ID NO: 762; SEQ ID NO: 763; SEQ ID NO: 764; SEQ ID NO: 765; SEQ ID NO: 766; SEQ ID NO: 767; SEQ ID NO: 768; SEQ ID NO: 769; SEQ ID NO: 770; SEQ ID NO: 771; SEQ ID NO: 772; SEQ ID NO: 773; SEQ ID NO: 774; SEQ ID NO: 775; SEQ ID NO: 776; SEQ ID NO: 777; SEQ ID NO: 778; SEQ ID NO: 779; SEQ ID NO: 780; SEQ ID NO: 781; SEQ ID NO: 782; SEQ ID NO: 783; SEQ ID NO: 784; SEQ ID NO: 785; SEQ ID NO: 786; SEQ ID NO: 787; SEQ ID NO: 788; SEQ ID NO: 789; SEQ ID NO: 790; SEQ ID NO: 791; SEQ ID NO: 792; SEQ ID NO: 793; SEQ ID NO: 794; SEQ ID NO: 795; SEQ ID NO: 796; SEQ ID NO: 797; SEQ ID NO: 798; SEQ ID NO: 799; SEQ ID NO: 800; SEQ ID NO: 801; SEQ ID NO: 802; SEQ ID NO: 803; SEQ ID NO: 804; SEQ ID NO: 805; SEQ ID NO: 806; SEQ ID NO: 807; SEQ ID NO: 808; SEQ ID NO: 809; SEQ ID NO: 810; SEQ ID NO: 811; SEQ ID NO: 812; SEQ ID NO: 813; SEQ ID NO: 814; SEQ ID NO: 815; SEQ ID NO: 816; SEQ ID NO: 817; SEQ ID NO: 818; SEQ ID NO: 819; SEQ ID NO: 820; SEQ ID NO: 821; SEQ ID NO: 822; SEQ ID NO: 823; SEQ ID NO: 824; SEQ ID NO: 825; SEQ ID NO: 826; SEQ ID NO: 827; SEQ ID NO: 828; SEQ ID NO: 829; SEQ ID NO: 830; SEQ ID NO: 831; SEQ ID NO: 832; SEQ ID NO: 833; SEQ ID NO: 834; SEQ ID NO: 835; SEQ ID NO: 836; SEQ ID NO: 837; SEQ ID NO: 838; SEQ ID NO: 839; SEQ ID NO: 840; SEQ ID NO: 841; SEQ ID NO: 842; SEQ ID NO: 843; SEQ ID NO: 844; SEQ ID NO: 845; SEQ ID NO: 846; SEQ ID NO: 847; SEQ ID NO: 848; SEQ ID NO: 849; SEQ ID NO: 850; SEQ ID NO: 851; SEQ ID NO: 852; SEQ ID NO: 853; SEQ ID NO: 854; SEQ ID NO: 855; SEQ ID NO: 856; SEQ ID NO: 857; SEQ ID NO: 858; SEQ ID NO: 859; SEQ ID NO: 860; SEQ ID NO: 861; SEQ ID NO: 862; SEQ ID NO: 863; SEQ ID NO: 864; SEQ ID NO: 865; SEQ ID NO: 866; SEQ ID NO: 867; SEQ ID NO: 868; SEQ ID NO: 869; SEQ ID NO: 870; SEQ ID NO: 871; SEQ ID NO: 872; SEQ ID NO: 873; SEQ ID NO: 874; SEQ ID NO: 875; SEQ ID NO: 876; SEQ ID NO: 877; SEQ ID NO: 878; SEQ ID NO: 879; SEQ ID NO: 880; SEQ ID NO: 881; SEQ ID NO: 882; SEQ ID NO: 883; SEQ ID NO: 884; SEQ ID NO: 885; SEQ ID NO: 886; SEQ ID NO: 887; SEQ ID NO: 888; SEQ ID NO: 889; SEQ ID NO: 890; SEQ ID NO: 891; SEQ ID NO: 892; SEQ ID NO: 893; SEQ ID NO: 894; SEQ ID NO: 895; SEQ ID NO: 896; SEQ ID NO: 897; SEQ ID NO: 898; SEQ ID NO: 899; SEQ ID NO: 900; SEQ ID NO: 901; SEQ ID NO: 902; SEQ ID NO: 903; SEQ ID NO: 904; SEQ ID NO: 905; SEQ ID NO: 906; SEQ ID NO: 907; SEQ ID NO: 908; SEQ ID NO: 909; SEQ ID NO: 910; SEQ ID NO: 911; SEQ ID NO: 912; SEQ ID NO: 913; SEQ ID NO: 914; SEQ ID NO: 915; SEQ ID NO: 916; SEQ ID NO: 917; SEQ ID NO: 918; SEQ ID NO: 919; SEQ ID NO: 920; SEQ ID NO: 921; SEQ ID NO: 922; SEQ ID NO: 923; SEQ ID NO: 924; SEQ ID NO: 925; SEQ ID NO: 926; SEQ ID NO: 927; SEQ ID NO: 928; SEQ ID NO: 929; SEQ ID NO: 930; SEQ ID NO: 931; SEQ ID NO: 932; SEQ ID NO: 933; SEQ ID NO: 934; SEQ ID NO: 935; SEQ ID NO: 936; SEQ ID NO: 937; SEQ ID NO: 938; SEQ ID NO: 939; SEQ ID NO: 940; SEQ ID NO: 941; SEQ ID NO: 942; SEQ ID NO: 943; SEQ ID NO: 944; SEQ ID NO: 945; SEQ ID NO: 946; SEQ ID NO: 947; SEQ ID NO: 948; SEQ ID NO: 949; SEQ ID NO: 950; SEQ ID NO: 951; SEQ ID NO: 952; SEQ ID NO: 953; SEQ ID NO: 954; SEQ ID NO: 955; SEQ ID NO: 956; SEQ ID NO: 957; SEQ ID NO: 958; SEQ ID NO: 959; SEQ ID NO: 960; SEQ ID NO: 961; SEQ ID NO: 962; SEQ ID NO: 963; SEQ ID NO: 964; SEQ ID NO: 965; SEQ ID NO: 966; SEQ ID NO: 967; SEQ ID NO: 968; SEQ ID NO: 969; SEQ ID NO: 970; SEQ ID NO: 971; SEQ ID NO: 972; SEQ ID NO: 973; SEQ ID NO: 974; SEQ ID NO: 975; SEQ ID NO: 976; SEQ ID NO: 977; SEQ ID NO: 978; SEQ ID NO: 979; SEQ ID NO: 980; SEQ ID NO: 981; SEQ ID NO: 982; SEQ ID NO: 983; SEQ ID NO: 984; SEQ ID NO: 985; SEQ ID NO: 986; SEQ ID NO: 987; SEQ ID NO: 988; SEQ ID NO: 989; SEQ ID NO: 990; SEQ ID NO: 991; SEQ ID NO: 992; SEQ ID NO: 993; SEQ ID NO: 994; SEQ ID NO: 995; SEQ ID NO: 996; SEQ ID NO: 997; SEQ ID NO: 998; SEQ ID NO: 999; SEQ ID NO: 1000; SEQ ID NO: 1001; SEQ ID NO: 1002; SEQ ID NO: 1003; SEQ ID NO: 1004; SEQ ID NO: 1005; SEQ ID NO: 1006; SEQ ID NO: 1007; SEQ ID NO: 1008; SEQ ID NO:1009; SEQ ID NO: 1010; SEQ ID NO:1011; SEQ ID NO: 1012; SEQ ID NO: 1013; SEQ ID NO: 1014; SEQ ID NO: 1015; SEQ ID NO: 1016; SEQ ID NO: 1017; SEQ ID NO: 1018; SEQ ID NO: 1019; SEQ ID NO: 1020; SEQ ID NO: 1021; SEQ ID NO: 1.022; SEQ ID NO: 1023; SEQ ID NO: 1024; SEQ ID NO: 1025; SEQ ID NO: 1026; SEQ ID NO: 1027; SEQ ID NO: 1028; SEQ ID NO: 1029; SEQ ID NO: 1030; SEQ ID NO: 1031; SEQ ID NO: 1032; SEQ ID NO: 1033; SEQ ID NO: 1034; SEQ ID NO: 1035; SEQ ID NO: 1036; SEQ ID NO: 1037; SEQ ID NO: 1038; SEQ ID NO: 1039; SEQ ID NO: 1040; SEQ ID NO: 1041; SEQ ID NO: 1042; SEQ ID NO: 1043; SEQ ID NO: 1044; SEQ ID NO: 1045; SEQ ID NO: 1046; SEQ ID NO: 1047; SEQ ID NO: 1048; SEQ ID NO: 1049; SEQ ID NO: 1050; SEQ ID NO: 1051; SEQ ID NO: 1052; SEQ ID NO: 1053; SEQ ID NO: 1054; SEQ ID NO: 1055; SEQ ID NO: 1056; SEQ ID NO: 1057; SEQ ID NO: 1058; SEQ ID NO: 1059; SEQ ID NO: 1060; SEQ ID NO: 1061; SEQ ID NO: 1062; SEQ ID NO: 1063; SEQ ID NO: 1064; SEQ ID NO: 1065; SEQ ID NO: 1066; SEQ ID NO: 1067; SEQ ID NO: 1068; SEQ ID NO: 1069; SEQ ID NO: 1070; SEQ ID NO: 1071; SEQ ID NO: 1072; SEQ ID NO: 1073; SEQ ID NO: 1074; SEQ ID NO: 1075; SEQ ID NO: 1076; SEQ ID NO: 1077; SEQ ID NO: 1078; SEQ ID NO: 1079; SEQ ID NO: 1080; SEQ ID NO: 1081; SEQ ID NO: 1082; SEQ ID NO: 1083; SEQ ID NO: 1084; SEQ ID NO: 1085; SEQ ID NO: 1086; SEQ ID NO: 1087; SEQ ID NO: 1088; SEQ ID NO: 1089; SEQ ID NO: 1090; SEQ ID NO: 1091; SEQ ID NO: 1092; SEQ ID NO: 1093; SEQ ID NO: 1094; SEQ ID NO: 1095; SEQ ID NO: 1096; SEQ ID NO: 1097; SEQ ID NO: 1098; SEQ ID NO: 1099; SEQ ID NO: 1100; SEQ ID NO: 1101; SEQ ID NO: 1102; SEQ ID NO: 1103; SEQ ID NO: 1104; SEQ ID NO: 1105; SEQ ID NO: 1106; SEQ ID NO: 1107; SEQ ID NO: 1108; SEQ ID NO:1109; SEQ ID NO: 1110; SEQ ID NO: 1111; SEQ ID NO: 1112; SEQ ID NO: 1113; SEQ ID NO: 1114; SEQ ID NO: 1115; SEQ ID NO: 1116; SEQ ID NO: 1117; SEQ ID NO: 1118; SEQ ID NO:1119; SEQ ID NO: 1120; SEQ ID NO: 1121; SEQ ID NO: 1122; SEQ ID NO: 1123; SEQ ID NO: 1124; SEQ ID NO: 1125; SEQ ID NO: 1126; SEQ ID NO: 1127; SEQ ID NO: 1128; SEQ ID NO: 1129; SEQ ID NO: 1130; SEQ ID NO: 1131; SEQ ID NO: 1132; SEQ ID NO: 1133; SEQ ID NO: 1134; SEQ ID NO: 1135; SEQ ID NO: 1136; SEQ ID NO: 1137; SEQ ID NO: 1138; SEQ ID NO: 1139; SEQ ID NO: 1140; SEQ ID NO: 1141; SEQ ID NO: 1142; SEQ ID NO: 1143; SEQ ID NO: 1144; SEQ ID NO: 1145; SEQ ID NO: 1146; SEQ ID NO: 1147; SEQ ID NO: 1148; SEQ ID NO: 1149; SEQ ID NO: 1150; SEQ ID NO: 1151; SEQ ID NO: 1152; SEQ ID NO: 1153; SEQ ID NO: 1154; SEQ ID NO: 1155; SEQ ID NO: 1156; SEQ ID NO: 1157; SEQ ID NO: 1158; SEQ ID NO: 1159; SEQ ID NO: 1160; SEQ ID NO: 1161; SEQ ID NO: 1162; SEQ ID NO: 1163; SEQ ID NO: 1164; SEQ ID NO: 1165; SEQ ID NO: 1166; SEQ ID NO: 1167; SEQ ID NO: 1168; SEQ ID NO: 1169; SEQ ID NO: 1170; SEQ ID NO: 1171; SEQ ID NO: 1172; SEQ ID NO: 1173; SEQ ID NO:1174; SEQ ID NO: 1175; SEQ ID NO:1176; SEQ ID NO: 1177; SEQ ID NO: 1178; SEQ ID NO: 1179; SEQ ID NO: 1180; SEQ ID NO: 1181; SEQ ID NO: 1182; SEQ ID NO: 1183; SEQ ID NO: 1184; SEQ ID NO: 1185; SEQ ID NO: 1186; SEQ ID NO: 1187; SEQ ID NO: 1188; SEQ ID NO: 1189; SEQ ID NO: 1190; SEQ ID NO: 1191; SEQ ID NO: 1192; SEQ ID NO: 1193; SEQ ID NO: 1194; SEQ ID NO: 1195; SEQ ID NO: 1196; SEQ ID NO: 1197; SEQ ID NO: 1198; SEQ ID NO: 1199; SEQ ID NO: 1200; SEQ ID NO: 1201; SEQ ID NO: 1202; SEQ ID NO: 1203; SEQ ID NO: 1204; SEQ ID NO: 1205; SEQ ID NO: 1206; SEQ ID NO: 1207; SEQ ID NO: 1208; SEQ ID NO: 1209; SEQ ID NO: 1210; SEQ ID NO: 1211; SEQ ID NO: 1212; SEQ ID NO: 1213; SEQ ID NO: 1214; SEQ ID NO: 1215; SEQ ID NO: 1216; SEQ ID NO: 1217; SEQ ID NO: 1218; SEQ ID NO: 1219; SEQ ID NO: 1220; SEQ ID NO: 1221; SEQ ID NO: 1222; SEQ ID NO: 1223; SEQ ID NO: 1224; SEQ ID NO: 1225; SEQ ID NO: 1226; SEQ ID NO: 1227; SEQ ID NO: 1228; SEQ ID NO: 1229; SEQ ID NO: 1230; SEQ ID NO: 1231; SEQ ID NO: 1232; SEQ ID NO: 1233; SEQ ID NO: 1234; SEQ ID NO: 1235; SEQ ID NO: 1236; SEQ ID NO: 1237; SEQ ID NO: 1238; SEQ ID NO: 1239; SEQ ID NO: 1240; SEQ ID NO: 1241; SEQ ID NO: 1242; SEQ ID NO: 1243; SEQ ID NO: 1244; SEQ ID NO: 1245; SEQ ID NO: 1246; SEQ ID NO: 1247; SEQ ID NO: 1248; SEQ ID NO: 1249; SEQ ID NO: 1250; SEQ ID NO: 1251; SEQ ID NO: 1252; SEQ ID NO: 1253; SEQ ID NO: 1254; SEQ ID NO: 1255; SEQ ID NO: 1256; SEQ ID NO: 1257; SEQ ID NO: 1258; SEQ ID NO: 1259; SEQ ID NO: 1260; SEQ ID NO: 1261; SEQ ID NO: 1262; SEQ ID NO: 1263; SEQ ID NO: 1264; SEQ ID NO: 1265; SEQ ID NO: 1266; SEQ ID NO: 1267; SEQ ID NO: 1268; SEQ ID NO: 1269; SEQ ID NO: 1270; SEQ ID NO: 1271; SEQ ID NO: 1272; SEQ ID NO: 1273; SEQ ID NO: 1274; SEQ ID NO: 1275; SEQ ID NO: 1276; SEQ ID NO: 1277; SEQ ID NO: 1278; SEQ ID NO: 1279; SEQ ID NO: 1280; SEQ ID NO: 1281; SEQ ID NO: 1282; SEQ ID NO: 1283; SEQ ID NO: 1284; SEQ ID NO: 1285; SEQ ID NO: 1286; SEQ ID NO: 1287; SEQ ID NO: 1288; SEQ ID NO: 1289; SEQ ID NO: 1290; SEQ ID NO: 1291; SEQ ID NO: 1292; SEQ ID NO: 1293; SEQ ID NO: 1294; SEQ ID NO: 1295; SEQ ID NO: 1296; SEQ ID NO: 1297; SEQ ID NO: 1298; SEQ ID NO: 1299; SEQ ID NO: 1300; SEQ ID NO: 1301; SEQ ID NO: 1302; SEQ ID NO: 1303; SEQ ID NO: 1304; SEQ ID NO: 1305; SEQ ID NO: 1306; SEQ ID NO: 1307; SEQ ID NO: 1308; SEQ ID NO: 1309; SEQ ID NO: 1310; SEQ ID NO: 1311I; SEQ ID NO: 1312; SEQ ID NO: 1313; SEQ ID NO: 1314; SEQ ID NO: 1315; SEQ ID NO: 1316; SEQ ID NO: 1317; SEQ ID NO: 1318; SEQ ID NO: 1319; SEQ ID NO: 1320; SEQ ID NO: 1321; SEQ ID NO: 1322; SEQ ID NO: 1323; SEQ ID NO: 1324; SEQ ID NO: 1325; SEQ ID NO: 1326; SEQ ID NO: 1327; SEQ ID NO: 1328; SEQ ID NO: 1329; SEQ ID NO: 1330; SEQ ID NO: 1331; SEQ ID NO: 1332; SEQ ID NO: 1333; SEQ ID NO: 1334; SEQ ID NO: 1335; SEQ ID NO: 1336; SEQ ID NO: 1337; SEQ ID NO: 1338; SEQ ID NO: 1339; SEQ ID NO: 1340; SEQ ID NO: 1341; SEQ ID NO: 1342; SEQ ID NO: 1343; SEQ ID NO: 1344; SEQ ID NO: 1345; SEQ ID NO: 1346; SEQ ID NO: 1347; SEQ ID NO: 1348; SEQ ID NO: 1349; SEQ ID NO: 1350; SEQ ID NO: 1351; SEQ ID NO: 1352; SEQ ID NO: 1353; SEQ ID NO: 1354; SEQ ID NO: 1355; SEQ ID NO: 1356; SEQ ID NO: 1357; SEQ ID NO: 1358; SEQ ID NO: 1359; SEQ ID NO: 1360; SEQ ID NO: 1361; SEQ ID NO: 1362; SEQ ID NO: 1363; SEQ ID NO: 1364; SEQ ID NO: 1365; SEQ ID NO: 1366; SEQ ID NO: 1367; SEQ ID NO: 1368; SEQ ID NO: 1369; SEQ ID NO: 1370; SEQ ID NO: 1371; SEQ ID NO: 1372; SEQ ID NO: 1373; SEQ ID NO: 1374; SEQ ID NO: 1375; SEQ ID NO: 1376; SEQ ID NO: 1377; SEQ ID NO: 1378; SEQ ID NO: 1379; SEQ ID NO: 1380; SEQ ID NO: 1381; SEQ ID NO: 1382; SEQ ID NO: 1383; SEQ ID NO: 1384; SEQ ID NO: 1385; SEQ ID NO: 1386; SEQ ID NO: 1387; SEQ ID NO: 1388; SEQ ID NO: 1389; SEQ ID NO: 1390; SEQ ID NO: 1391; SEQ ID NO: 1392; SEQ ID NO: 1393; SEQ ID NO: 1394; SEQ ID NO: 1395; SEQ ID NO: 1396; SEQ ID NO: 1397; SEQ ID NO: 1398; SEQ ID NO: 1399; SEQ ID NO: 1400; SEQ ID NO: 1401; SEQ ID NO: 1402; SEQ ID NO: 1403; SEQ ID NO: 1404; SEQ ID NO: 1405; SEQ ID NO: 1406; SEQ ID NO: 1407; SEQ ID NO: 1408; SEQ ID NO: 1409; SEQ ID NO: 1410; SEQ ID NO: 1411; SEQ ID NO: 1412; SEQ ID NO: 1413; SEQ ID NO: 1414; SEQ ID NO: 1415; SEQ ID NO: 1416; SEQ ID NO: 1417; SEQ ID NO: 1418; SEQ ID NO: 1419; SEQ ID NO: 1420; SEQ ID NO: 1421; SEQ ID NO: 1422; SEQ ID NO: 1423; SEQ ID NO: 1424; SEQ ID NO: 1425; SEQ ID NO: 1426; SEQ ID NO: 1427; SEQ ID NO: 1428; SEQ ID NO: 1429; SEQ ID NO: 1430; SEQ ID NO: 1431; SEQ ID NO: 1432; SEQ ID NO: 1433; SEQ ID NO: 1434; SEQ ID NO: 1435; SEQ ID NO: 1436; SEQ ID NO: 1437; SEQ ID NO: 1438; SEQ ID NO: 1439; SEQ ID NO: 1440; SEQ ID NO: 1441; SEQ ID NO: 1442; SEQ ID NO: 1443; SEQ ID NO: 1444; SEQ ID NO: 1445; SEQ ID NO: 1446; SEQ ID NO: 1447; SEQ ID NO: 1448; SEQ ID NO: 1449; SEQ ID NO: 1450; SEQ ID NO: 1451; SEQ ID NO: 1452; SEQ ID NO: 1453; SEQ ID NO: 1454; SEQ ID NO: 1455; SEQ ID NO: 1456; SEQ ID NO: 1457; SEQ ID NO: 1458; SEQ ID NO: 1459; SEQ ID NO: 1460; SEQ ID NO: 1461; SEQ ID NO: 1462; SEQ ID NO: 1463; SEQ ID NO: 1464; SEQ ID NO: 1465; SEQ ID NO: 1466; SEQ ID NO: 1467; SEQ ID NO: 1468; SEQ ID NO: 1469; SEQ ID NO: 1470; SEQ ID NO: 1471; SEQ ID NO: 1472; SEQ ID NO: 1473; SEQ ID NO: 1474; SEQ ID NO: 1475; SEQ ID NO: 1476; SEQ ID NO: 1477; SEQ ID NO: 1478; SEQ ID NO: 1479; SEQ ID NO: 1480; SEQ ID NO: 1481; SEQ ID NO: 1482; SEQ ID NO: 1483; SEQ ID NO: 1484; SEQ ID NO: 1485; SEQ ID NO: 1486; SEQ ID NO: 1487; SEQ ID NO: 1488; SEQ ID NO: 1489; SEQ ID NO: 1490; SEQ ID NO: 1491; SEQ ID NO: 1492; SEQ ID NO: 1493; SEQ ID NO: 1494; SEQ ID NO: 1495; SEQ ID NO: 1496; SEQ ID NO: 1497; SEQ ID NO: 1498; SEQ ID NO: 1499; SEQ ID NO: 1500; SEQ ID NO: 1501; SEQ ID NO: 1502; SEQ ID NO: 1503; SEQ ID NO: 1504; SEQ ID NO: 1505; SEQ ID NO: 1506; SEQ ID NO: 1507; SEQ ID NO: 1508; SEQ ID NO: 1509; SEQ ID NO: 1510; SEQ ID NO: 1511; SEQ ID NO: 1512; SEQ ID NO: 1513; SEQ ID NO: 1514; SEQ ID NO: 1515; SEQ ID NO: 1516; SEQ ID NO: 1517; SEQ ID NO: 1518; SEQ ID NO: 1519; SEQ ID NO: 1520; SEQ ID NO: 1521; SEQ ID NO: 1522; SEQ ID NO: 1523; SEQ ID NO: 1524; SEQ ID NO: 1525; SEQ ID NO: 1526; SEQ ID NO: 1527; SEQ ID NO: 1528; SEQ ID NO: 1529; SEQ ID NO: 1530; SEQ ID NO: 1531; SEQ ID NO: 1532; SEQ ID NO: 1533; SEQ ID NO: 1534; SEQ ID NO: 1535; SEQ ID NO: 1536; SEQ ID NO: 1537; SEQ ID NO: 1538; SEQ ID NO: 1539; SEQ ID NO: 1540; SEQ ID NO: 1541; SEQ ID NO: 1542; SEQ ID NO: 1543; SEQ ID NO: 1544; SEQ ID NO: 1545; SEQ ID NO: 1546; SEQ ID NO: 1547; SEQ ID NO: 1548; SEQ ID NO: 1549; SEQ ID NO: 1550; SEQ ID NO: 1551; SEQ ID NO: 1552; SEQ ID NO: 1553; SEQ ID NO: 1554; SEQ ID NO: 1555; SEQ ID NO: 1556; SEQ ID NO: 1557; SEQ ID NO: 1558; SEQ ID NO: 1559; SEQ ID NO: 1560; SEQ ID NO: 1561; SEQ ID NO: 1562; SEQ ID NO: 1563; SEQ ID NO: 1564; SEQ ID NO: 1565; SEQ ID NO: 1566; SEQ ID NO: 1567; SEQ ID NO: 1568; SEQ ID NO: 1569; SEQ ID NO: 1570; SEQ ID NO: 1571; SEQ ID NO: 1572; SEQ ID NO: 1573; SEQ ID NO: 1574; SEQ ID NO: 1575; SEQ ID NO: 9525; SEQ ID NO: 9526; SEQ ID NO: 9527; SEQ ID NO: 9528; SEQ ID NO: 9529; SEQ ID NO: 9530; SEQ ID NO: 9531; SEQ ID NO: 9532; SEQ ID NO: 9533; SEQ ID NO: 9534; SEQ ID NO: 9535; SEQ ID NO: 9536; SEQ ID NO: 9537; SEQ ID NO: 9538; SEQ ID NO: 9539; SEQ ID NO: 9540; SEQ ID NO: 9541; SEQ ID NO: 9542; SEQ ID NO: 9543; SEQ ID NO: 9544; SEQ ID NO: 9545; SEQ ID NO: 9546; SEQ ID NO: 9547; SEQ ID NO: 9548; SEQ ID NO: 9549; SEQ ID NO: 9550; SEQ ID NO: 9551; SEQ ID NO: 9552; SEQ ID NO: 9553; SEQ ID NO: 9554; SEQ ID NO: 9555; SEQ ID NO: 9556; SEQ ID NO: 9557; SEQ ID NO: 9558; SEQ ID NO: 9559; SEQ ID NO: 9560; SEQ ID NO: 9561; SEQ ID NO: 9562; SEQ ID NO: 9563; SEQ ID NO: 9564; SEQ ID NO: 9565; SEQ ID NO: 9566; SEQ ID NO: 9567; SEQ ID NO: 9568; SEQ ID NO: 9569; SEQ ID NO: 9570; SEQ ID NO: 9571; SEQ ID NO: 9572; SEQ ID NO: 9573; SEQ ID NO: 9574; SEQ ID NO: 9575; SEQ ID NO: 9576; SEQ ID NO: 9577; SEQ ID NO: 9578; SEQ ID NO: 9579; SEQ ID NO: 9580; SEQ ID NO: 9581; SEQ ID NO: 9582; SEQ ID NO: 9583; SEQ ID NO: 9584; SEQ ID NO: 9585; SEQ ID NO: 9586; SEQ ID NO: 9587; SEQ ID NO: 9588; SEQ ID NO: 9589; SEQ ID NO: 9590; SEQ ID NO: 9591 and SEQ ID NO: 9592.

In one embodiment, the H. pylori cell envelope polypeptide or a fragment thereof is an H. pylori flagella-associated polypeptide or a fragment thereof encoded by a nucleic acid having a nucleotide sequence selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO:97; SEQ ID NO: 98; SEQ ID NO: 99; SEQ ID NO: 100; SEQ ID NO: 101; SEQ ID NO: 102; SEQ ID NO: 103; SEQ ID NO: 9525; SEQ ID NO: 9526 and SEQ ID NO: 9527.

In another embodiment, the H. pylori cell envelope polypeptide or a fragment thereof is an H. pylori outer membrane polypeptide or a fragment thereof encoded by a nucleic acid having a nucleotide sequence selected from the group consisting of SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO:111; SEQ ID NO: 112; SEQ ID NO: 113; SEQ ID NO: 114; SEQ ID NO: 115; SEQ ID NO: 116; SEQ ID NO: 117; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145: SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; SEQ ID NO: 186; SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; SEQ ID NO: 211; SEQ ID NO: 212; SEQ ID NO: 213; SEQ ID NO: 214; SEQ ID NO: 215; SEQ ID NO: 216; SEQ ID NO: 217; SEQ ID NO: 218; SEQ ID NO: 219; SEQ ID NO: 220; SEQ ID NO: 221; SEQ ID NO: 222; SEQ ID NO: 223; SEQ ID NO: 224; SEQ ID NO: 225; SEQ ID NO: 226; SEQ ID NO: 227; SEQ ID NO: 228; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 236; SEQ ID NO: 237; SEQ ID NO: 238; SEQ ID NO: 239; SEQ ID NO: 240; SEQ ID NO: 241; SEQ ID NO: 242; SEQ ID NO: 243; SEQ ID NO: 244; SEQ ID NO: 245; SEQ ID NO: 246; SEQ ID NO: 247; SEQ ID NO: 248; SEQ ID NO: 249; SEQ ID NO: 250; SEQ ID NO:251; SEQ ID NO: 252; SEQ ID NO: 253; SEQ ID NO: 254; SEQ ID NO: 255; SEQ ID NO: 256; SEQ ID NO: 257; SEQ ID NO: 258; SEQ ID NO: 259; SEQ ID NO: 260; SEQ ID NO: 261; SEQ ID NO: 262; SEQ ID NO: 263; SEQ ID NO: 264; SEQ ID NO: 265; SEQ ID NO: 266; SEQ ID NO: 267; SEQ ID NO: 268; SEQ ID NO: 269; SEQ ID NO: 270; SEQ ID NO: 271; SEQ ID NO: 272; SEQ ID NO: 273; SEQ ID NO: 274; SEQ ID NO: 275; SEQ ID NO: 276; SEQ ID NO: 277; SEQ ID NO: 278; SEQ ID NO: 279; SEQ ID NO: 280; SEQ ID NO: 281; SEQ ID NO: 282; SEQ ID NO: 283; SEQ ID NO: 284; SEQ ID NO: 285; SEQ ID NO: 286; SEQ ID NO: 287; SEQ ID NO: 288; SEQ ID NO: 289; SEQ ID NO: 290; SEQ ID NO: 291; SEQ ID NO: 292; SEQ ID NO: 293; SEQ ID NO: 294; SEQ ID NO: 295; SEQ ID NO: 296; SEQ ID NO: 297; SEQ ID NO: 298; SEQ ID NO: 299; SEQ ID NO: 300; SEQ ID NO: 301; SEQ ID NO: 302; SEQ ID NO: 303; SEQ ID NO: 304; SEQ ID NO: 305; SEQ ID NO: 306; SEQ ID NO: 307; SEQ ID NO: 308; SEQ ID NO: 309; SEQ ID NO: 310; SEQ ID NO: 311; SEQ ID NO:312; SEQ ID NO:313; SEQ ID NO: 314; SEQ ID NO: 315; SEQ ID NO: 316; SEQ ID NO: 317; SEQ ID NO: 318; SEQ ID NO: 319; SEQ ID NO: 320; SEQ ID NO: 321; SEQ ID NO: 322; SEQ ID NO: 323; SEQ ID NO: 324; SEQ ID NO: 325; SEQ ID NO: 326; SEQ ID NO: 327; SEQ ID NO: 328; SEQ ID NO: 329; SEQ ID NO: 330; SEQ ID NO: 331; SEQ ID NO: 332; SEQ ID NO: 333; SEQ ID NO: 334; SEQ ID NO: 335; SEQ ID NO: 336; SEQ ID NO: 337; SEQ ID NO: 338; SEQ ID NO: 339; SEQ ID NO: 340; SEQ ID NO: 341; SEQ ID NO: 342; SEQ ID NO: 343; SEQ ID NO: 344; SEQ ID NO: 345; SEQ ID NO: 346; SEQ ID NO: 347; SEQ ID NO: 348; SEQ ID NO: 349; SEQ ID NO: 350; SEQ ID NO: 351; SEQ ID NO: 352; SEQ ID NO: 353; SEQ ID NO: 354; SEQ ID NO: 355; SEQ ID NO: 356; SEQ ID NO: 357; SEQ ID NO: 358; SEQ ID NO: 359; SEQ ID NO: 360; SEQ ID NO: 361; SEQ ID NO: 362; SEQ ID NO: 363; SEQ ID NO: 364; SEQ ID NO: 365; SEQ ID NO: 366; SEQ ID NO: 367; SEQ ID NO: 368; SEQ ID NO: 369; SEQ ID NO: 370; SEQ ID NO: 371; SEQ ID NO: 372; SEQ ID NO: 373; SEQ ID NO: 374; SEQ ID NO: 375; SEQ ID NO: 376; SEQ ID NO: 377; SEQ ID NO: 378; SEQ ID NO: 379; SEQ ID NO: 380; SEQ ID NO: 381; SEQ ID NO: 382; SEQ ID NO: 383; SEQ ID NO: 384; SEQ ID NO: 385; SEQ ID NO: 386; SEQ ID NO: 387; SEQ ID NO: 388; SEQ ID NO: 389; SEQ ID NO: 390; SEQ ID NO: 391; SEQ ID NO: 392; SEQ ID NO: 393; SEQ ID NO: 394; SEQ ID NO: 395; SEQ ID NO: 396; SEQ ID NO: 397; SEQ ID NO: 398; SEQ ID NO: 399; SEQ ID NO: 400; SEQ ID NO: 401; SEQ ID NO: 402; SEQ ID NO: 403; SEQ ID NO: 404; SEQ ID NO: 405; SEQ ID NO: 406; SEQ ID NO: 407; SEQ ID NO: 408; SEQ ID NO: 409; SEQ ID NO: 410; SEQ ID NO: 411; SEQ ID NO: 412; SEQ ID NO: 413; SEQ ID NO: 414; SEQ ID NO: 415; SEQ ID NO: 416; SEQ ID NO: 417; SEQ ID NO: 418; SEQ ID NO: 419; SEQ ID NO: 420; SEQ ID NO: 421; SEQ ID NO: 422; SEQ ID NO: 423; SEQ ID NO: 424; SEQ ID NO: 425; SEQ ID NO: 426; SEQ ID NO: 427; SEQ ID NO: 428; SEQ ID NO: 429; SEQ ID NO: 430; SEQ ID NO: 431; SEQ ID NO: 432; SEQ ID NO: 433; SEQ ID NO: 434; SEQ ID NO: 435; SEQ ID NO: 436; SEQ ID NO: 437; SEQ ID NO: 438; SEQ ID NO: 439; SEQ ID NO: 440; SEQ ID NO: 441; SEQ ID NO: 442; SEQ ID NO: 443; SEQ ID NO: 444; SEQ ID NO: 445; SEQ ID NO: 446; SEQ ID NO: 447; SEQ ID NO: 448; SEQ ID NO: 449; SEQ ID NO: 450; SEQ ID NO: 451; SEQ ID NO: 452; SEQ ID NO: 453; SEQ ID NO: 454; SEQ ID NO: 455; SEQ ID NO: 456; SEQ ID NO: 457; SEQ ID NO: 458; SEQ ID NO: 459; SEQ ID NO: 460; SEQ ID NO: 461; SEQ ID NO: 462; SEQ ID NO: 463; SEQ ID NO: 464; SEQ ID NO: 465; SEQ ID NO: 466; SEQ ID NO: 467; SEQ ID NO: 468; SEQ ID NO: 469; SEQ ID NO: 470; SEQ ID NO: 471; SEQ ID NO: 472; SEQ ID NO: 473; SEQ ID NO: 474; SEQ ID NO: 475; SEQ ID NO: 476; SEQ ID NO: 477; SEQ ID NO: 478; SEQ ID NO: 479; SEQ ID NO: 480; SEQ ID NO: 481; SEQ ID NO: 482; SEQ ID NO: 483; SEQ ID NO: 484; SEQ ID NO: 485; SEQ ID NO: 486; SEQ ID NO: 487; SEQ ID NO: 488; SEQ ID NO: 489; SEQ ID NO: 490; SEQ ID NO: 491; SEQ ID NO: 492; SEQ ID NO: 493; SEQ ID NO: 494; SEQ ID NO: 495; SEQ ID NO: 496; SEQ ID NO: 497; SEQ ID NO: 498; SEQ ID NO: 499; SEQ ID NO: 500; SEQ ID NO: 501; SEQ ID NO: 502; SEQ ID NO: 503; SEQ ID NO: 504; SEQ ID NO: 505; SEQ ID NO: 506; SEQ ID NO: 507; SEQ ID NO: 508; SEQ ID NO: 509; SEQ ID NO: 510; SEQ ID NO: 9528; SEQ ID NO: 9529; SEQ ID NO: 9530; SEQ ID NO: 9531; SEQ ID NO: 9532; SEQ ID NO: 9533; SEQ ID NO: 9534; SEQ ID NO: 9535 and SEQ ID NO: 9536.

In another embodiment, the H. pylori outer membrane polypeptide or a fragment thereof is an H. pylori polypeptide having a terminal phenylalanine residue or a fragment thereof encoded by a nucleic acid having a nucleotide sequence selected from the group consisting of SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO: 117; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; SEQ ID NO: 186; SEQ ID NO: 187; SEQ ID NO: 188; SEQ ID NO: 189; SEQ ID NO: 190, SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; SEQ ID NO: 211; SEQ ID NO: 212; SEQ ID NO: 213; SEQ ID NO: 214; SEQ ID NO: 215; SEQ ID NO: 216; SEQ ID NO: 217; SEQ ID NO: 218; SEQ ID NO: 219; SEQ ID NO: 220; SEQ ID NO: 221; SEQ ID NO: 222; SEQ ID NO: 223; SEQ ID NO: 224; SEQ ID NO: 225; SEQ ID NO: 226; SEQ ID NO: 227; SEQ ID NO: 228; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 236; SEQ ID NO: 237; SEQ ID NO: 238; SEQ ID NO: 239; SEQ ID NO: 240; SEQ ID NO: 241; SEQ ID NO: 242; SEQ ID NO: 243; SEQ ID NO: 244; SEQ ID NO: 245; SEQ ID NO: 246; SEQ ID NO: 247; SEQ ID NO: 248; SEQ ID NO: 249; SEQ ID NO: 250; SEQ ID NO: 251; SEQ ID NO: 252; SEQ ID NO: 253; SEQ ID NO: 254; SEQ ID NO: 255; SEQ ID NO: 256; SEQ ID NO: 257; SEQ ID NO: 258; SEQ ID NO: 259; SEQ ID NO: 260; SEQ ID NO: 261; SEQ ID NO: 262; SEQ ID NO: 263; SEQ ID NO: 264; SEQ ID NO: 265; SEQ ID NO: 266; SEQ ID NO: 267; SEQ ID NO: 268; SEQ ID NO: 269; SEQ ID NO: 270; SEQ ID NO: 271; SEQ ID NO: 272; SEQ ID NO: 273; SEQ ID NO: 274; SEQ ID NO: 275; SEQ ID NO: 276; SEQ ID NO: 277; SEQ ID NO: 278; SEQ ID NO: 279; SEQ ID NO: 280; SEQ ID NO: 281; SEQ ID NO: 282; SEQ ID NO: 283; SEQ ID NO: 284; SEQ ID NO: 285; SEQ ID NO: 286; SEQ ID NO: 287; SEQ ID NO: 288; SEQ ID NO: 289; SEQ ID NO: 290; SEQ ID NO: 291; SEQ ID NO: 292; SEQ ID NO: 293; SEQ ID NO: 294; SEQ ID NO: 295; SEQ ID NO: 296; SEQ ID NO: 297; SEQ ID NO: 298; SEQ ID NO: 299; SEQ ID NO: 300; SEQ ID NO: 301; SEQ ID NO: 302; SEQ ID NO: 303; SEQ ID NO: 304; SEQ ID NO: 305; SEQ ID NO: 306; SEQ ID NO: 307; SEQ ID NO: 308; SEQ ID NO: 309; SEQ ID NO: 310; SEQ ID NO: 311; SEQ ID NO: 312; SEQ ID NO: 313; SEQ ID NO: 314; SEQ ID NO: 315; SEQ ID NO: 316; SEQ ID NO: 317; SEQ ID NO: 318; SEQ ID NO: 319; SEQ ID NO: 320; SEQ ID NO: 321; SEQ ID NO: 322; SEQ ID NO: 323; SEQ ID NO: 324; SEQ ID NO: 325 SEQ ID NO: 326; SEQ ID NO: 327; SEQ ID NO: 328; SEQ ID NO: 329; SEQ ID NO: 330; SEQ ID NO: 331; SEQ ID NO: 332; SEQ ID NO: 333; SEQ ID NO: 342; SEQ ID NO: 343; SEQ ID NO: 344; SEQ ID NO: 345; SEQ ID NO: 346; SEQ ID NO: 347; SEQ ID NO: 348; SEQ ID NO: 349; SEQ ID NO: 350; SEQ ID NO: 351; SEQ ID NO: 352; SEQ ID NO: 353; SEQ ID NO: 354; SEQ ID NO: 355; SEQ ID NO: 356; SEQ ID NO: 357; SEQ ID NO: 358; SEQ ID NO: 359; SEQ ID NO: 360; SEQ ID NO: 361; SEQ ID NO: 362; SEQ ID NO: 369; SEQ ID NO: 370; SEQ ID NO: 371; SEQ ID NO: 372; SEQ ID NO: 373; SEQ ID NO: 374; SEQ ID NO: 375; SEQ ID NO: 376; SEQ ID NO: 377; SEQ ID NO: 378; SEQ ID NO: 379; SEQ ID NO: 380; SEQ ID NO: 381; SEQ ID NO: 382; SEQ ID NO: 383; SEQ ID NO: 384; SEQ ID NO: 385; SEQ ID NO: 386; SEQ ID NO: 387; SEQ ID NO: 388; SEQ ID NO: 389; SEQ ID NO: 390; SEQ ID NO: 391; SEQ ID NO: 392; SEQ ID NO: 393; SEQ ID NO: 394; SEQ ID NO: 395; SEQ ID NO: 396; SEQ ID NO: 397; SEQ ID NO: 9528; SEQ ID NO: 9529; SEQ ID NO: 9530; SEQ ID NO: 9531; SEQ ID NO: 9532; SEQ ID NO: 9533; SEQ ID NO: 9534; SEQ ID NO: 9535 and SEQ ID NO: 9536.

In another embodiment, the H. pylori outer membrane polypeptide or a fragment thereof is an H. pylori polypeptide having a terminal phenylalanine residue and a C-terminal tyrosine cluster or a fragment thereof encoded by a nucleic acid having a nucleotide sequence selected from the group consisting of SEQ ID NO: 326; SEQ ID NO: 327; SEQ ID NO: 328; SEQ ID NO: 329; SEQ ID NO: 330; SEQ ID NO: 331; SEQ ID NO: 332; SEQ ID NO: 333; SEQ ID NO: 342; SEQ ID NO: 343; SEQ ID NO: 344; SEQ ID NO: 345; SEQ ID NO: 346; SEQ ID NO: 347; SEQ ID NO: 348; SEQ ID NO: 349; SEQ ID NO: 350; SEQ ID NO: 351; SEQ ID NO: 352; SEQ ID NO: 353; SEQ ID NO: 354; SEQ ID NO: 355; SEQ ID NO: 356; SEQ ID NO: 357; SEQ ID NO: 358; SEQ ID NO: 359; SEQ ID NO: 360; SEQ ID NO: 361; SEQ ID NO: 362; SEQ ID NO: 369; SEQ ID NO: 370; SEQ ID NO: 371; SEQ ID NO: 372; SEQ ID NO: 373; SEQ ID NO: 374; SEQ ID NO: 375; SEQ ID NO: 376; SEQ ID NO: 377; SEQ ID NO: 378; SEQ ID NO: 379; SEQ ID NO: 380; SEQ ID NO: 381; SEQ ID NO: 382; SEQ ID NO: 383; SEQ ID NO: 384; SEQ ID NO: 385; SEQ ID NO: 386; SEQ ID NO: 387; SEQ ID NO: 388; SEQ ID NO: 389; SEQ ID NO: 390; SEQ ID NO: 391; SEQ ID NO: 392; SEQ ID NO: 393; SEQ ID NO: 394; SEQ ID NO: 395; SEQ ID NO: 396; SEQ ID NO: 397 and SEQ ID NO: 9536.

In another embodiment, the H. pylori outer membrane polypeptide or a fragment thereof is an H. pylori polypeptide having a terminal tyrosine cluster motif or a fragment thereof encoded by a nucleic acid having a nucleotide sequence selected from the group consisting of SEQ ID NO: 326; SEQ ID NO: 327; SEQ ID NO: 328; SEQ ID NO: 329; SEQ ID NO: 330; SEQ ID NO: 331; SEQ ID NO: 332; SEQ ID NO: 333; SEQ ID NO: 334; SEQ ID NO: 335; SEQ ID NO: 336; SEQ ID NO: 337; SEQ ID NO: 338; SEQ ID NO: 339; SEQ ID NO: 340; SEQ ID NO: 341; SEQ ID NO: 342; SEQ ID NO: 343; SEQ ID NO: 344; SEQ ID NO: 345; SEQ ID NO: 346; SEQ ID NO: 347; SEQ ID NO: 348; SEQ ID NO: 349; SEQ ID NO: 350; SEQ ID NO: 351; SEQ ID NO: 352; SEQ ID NO: 353; SEQ ID NO: 354; SEQ ID NO: 355; SEQ ID NO: 356; SEQ ID NO: 357; SEQ ID NO: 358; SEQ ID NO: 359; SEQ ID NO: 360; SEQ ID NO: 361; SEQ ID NO: 362; SEQ ID NO: 363; SEQ ID NO: 364; SEQ ID NO: 365; SEQ ID NO: 366; SEQ ID NO: 367; SEQ ID NO: 368; SEQ ID NO: 369; SEQ ID NO: 370; SEQ ID NO: 371; SEQ ID NO: 372; SEQ ID NO: 373; SEQ ID NO: 374; SEQ ID NO: 375; SEQ ID NO: 376; SEQ ID NO: 377; SEQ ID NO: 378; SEQ ID NO: 379; SEQ ID NO: 380; SEQ ID NO: 381; SEQ ID NO: 382; SEQ ID NO: 383; SEQ ID NO: 384; SEQ ID NO: 385; SEQ ID NO: 386; SEQ ID NO: 387; SEQ ID NO: 388; SEQ ID NO: 389; SEQ ID NO: 390; SEQ ID NO: 391; SEQ ID NO: 392; SEQ ID NO: 393; SEQ ID NO: 394; SEQ ID NO: 395; SEQ ID NO: 396; SEQ ID NO: 397; SEQ ID NO: 398; SEQ ID NO: 399; SEQ ID NO: 400; SEQ ID NO: 401; SEQ ID NO: 402; SEQ ID NO: 403; SEQ ID NO: 404; SEQ ID NO: 405; SEQ ID NO: 406; SEQ ID NO: 407; SEQ ID NO: 408; SEQ ID NO: 409; SEQ ID NO: 410; SEQ ID NO: 411; SEQ ID NO: 412; SEQ ID NO: 413; SEQ ID NO: 414; SEQ ID NO: 415; SEQ ID NO: 416; SEQ ID NO: 424; SEQ ID NO: 425 and SEQ ID NO: 9536.

In another embodiment, the H. pylori cell envelope polypeptide or a fragment thereof is an H. pylori inner membrane polypeptide or a fragment thereof encoded by a nucleic acid having a nucleotide sequence selected from the group consisting of SEQ ID NO: 511; SEQ ID NO: 512; SEQ ID NO: 513; SEQ ID NO: 514; SEQ ID NO: 515; SEQ ID NO: 516; SEQ ID NO: 517; SEQ ID NO: 518; SEQ ID NO: 519; SEQ ID NO: 520; SEQ ID NO: 521; SEQ ID NO: 522; SEQ ID NO: 523; SEQ ID NO: 524; SEQ ID NO: 525; SEQ ID NO: 526; SEQ ID NO: 527; SEQ ID NO: 528; SEQ ID NO: 529; SEQ ID NO: 530; SEQ ID NO: 531; SEQ ID NO: 532; SEQ ID NO: 533; SEQ ID NO: 534; SEQ ID NO: 535; SEQ ID NO: 536; SEQ ID NO: 537; SEQ ID NO: 538; SEQ ID NO: 539; SEQ ID NO: 540; SEQ ID NO: 541; SEQ ID NO: 542; SEQ ID NO: 543; SEQ ID NO: 544; SEQ ID NO: 545; SEQ ID NO: 546; SEQ ID NO: 547; SEQ ID NO: 548; SEQ ID NO: 549; SEQ ID NO: 550; SEQ ID NO: 551; SEQ ID NO: 552; SEQ ID NO: 553; SEQ ID NO: 554; SEQ ID NO: 555; SEQ ID NO: 556; SEQ ID NO: 557; SEQ ID NO: 558; SEQ ID NO: 559; SEQ ID NO: 560; SEQ ID NO: 561; SEQ ID NO: 562; SEQ ID NO: 563; SEQ ID NO: 564; SEQ ID NO: 565; SEQ ID NO: 566; SEQ ID NO: 567; SEQ ID NO: 568; SEQ ID NO: 569; SEQ ID NO: 570; SEQ ID NO: 571; SEQ ID NO: 572; SEQ ID NO: 573; SEQ ID NO: 574; SEQ ID NO: 575; SEQ ID NO: 576; SEQ ID NO: 577; SEQ ID NO: 578; SEQ ID NO: 579; SEQ ID NO: 580; SEQ ID NO: 581; SEQ ID NO: 582; SEQ ID NO: 583; SEQ ID NO: 584; SEQ ID NO: 585; SEQ ID NO: 586; SEQ ID NO: 587; SEQ ID NO: 588; SEQ ID NO: 589; SEQ ID NO: 590; SEQ ID NO: 591; SEQ ID NO: 592; SEQ ID NO: 593; SEQ ID NO: 594; SEQ ID NO: 595; SEQ ID NO: 596; SEQ ID NO: 597; SEQ ID NO: 598; SEQ ID NO: 599; SEQ ID NO: 600; SEQ ID NO: 601; SEQ ID NO: 602; SEQ ID NO: 603; SEQ ID NO: 604; SEQ ID NO: 605; SEQ ID NO: 606; SEQ ID NO: 607; SEQ ID NO: 608; SEQ ID NO: 609; SEQ ID NO: 610; SEQ ID NO: 611; SEQ ID NO: 612; SEQ ID NO: 613; SEQ ID NO: 614; SEQ ID NO: 615; SEQ ID NO: 616; SEQ ID NO: 617; SEQ ID NO: 618; SEQ ID NO: 619; SEQ ID NO: 620; SEQ ID NO: 621; SEQ ID NO: 622; SEQ ID NO: 623; SEQ ID NO: 624; SEQ ID NO: 625; SEQ ID NO: 626; SEQ ID NO: 627; SEQ ID NO: 628; SEQ ID NO: 629; SEQ ID NO: 630; SEQ ID NO: 631; SEQ ID NO: 632; SEQ ID NO: 633; SEQ ID NO: 634; SEQ ID NO: 635; SEQ ID NO: 636; SEQ ID NO: 637; SEQ ID NO: 638; SEQ ID NO: 639; SEQ ID NO: 640; SEQ ID NO: 641; SEQ ID NO: 642; SEQ ID NO: 643; SEQ ID NO: 644; SEQ ID NO: 645; SEQ ID NO: 646; SEQ ID NO: 647; SEQ ID NO: 648; SEQ ID NO: 649; SEQ ID NO: 650; SEQ ID NO: 651; SEQ ID NO: 652; SEQ ID NO: 653; SEQ ID NO: 654; SEQ ID NO: 655; SEQ ID NO: 656; SEQ ID NO: 657; SEQ ID NO: 658; SEQ ID NO: 659; SEQ ID NO: 660; SEQ ID NO: 661; SEQ ID NO: 662; SEQ ID NO: 663; SEQ ID NO: 664; SEQ ID NO: 665; SEQ ID NO: 666; SEQ ID NO: 667; SEQ ID NO: 668; SEQ ID NO: 669; SEQ ID NO: 670; SEQ ID NO: 671; SEQ ID NO: 672; SEQ ID NO: 673; SEQ ID NO: 674; SEQ ID NO: 675; SEQ ID NO: 676; SEQ ID NO: 677; SEQ ID NO: 678; SEQ ID NO: 678; SEQ ID NO: 680; SEQ ID NO: 681; SEQ ID NO: 682; SEQ ID NO: 683; SEQ ID NO: 684; SEQ ID NO: 685; SEQ ID NO: 686; SEQ ID NO: 687; SEQ ID NO: 688; SEQ ID NO: 689; SEQ ID NO: 690; SEQ ID NO: 691; SEQ ID NO: 692; SEQ ID NO: 693; SEQ ID NO: 694; SEQ ID NO: 695; SEQ ID NO: 696; SEQ ID NO: 697; SEQ ID NO: 698; SEQ ID NO: 699; SEQ ID NO: 700; SEQ ID NO: 701; SEQ ID NO: 702; SEQ ID NO: 703; SEQ ID NO: 704; SEQ ID NO: 705; SEQ ID NO: 706; SEQ ID NO: 707; SEQ ID NO: 708; SEQ ID NO: 709; SEQ ID NO: 710; SEQ ID NO: 711; SEQ ID NO: 712; SEQ ID NO: 713; SEQ ID NO: 714; SEQ ID NO: 715; SEQ ID NO: 716; SEQ ID NO: 717; SEQ ID NO: 718; SEQ ID NO: 719; SEQ ID NO: 720; SEQ ID NO: 721; SEQ ID NO: 722; SEQ ID NO: 723; SEQ ID NO: 724; SEQ ID NO: 725; SEQ ID NO: 726; SEQ ID NO: 727; SEQ ID NO: 728; SEQ ID NO: 729; SEQ ID NO: 730; SEQ ID NO: 731; SEQ ID NO: 732; SEQ ID NO: 733; SEQ ID NO: 734; SEQ ID NO: 735; SEQ ID NO: 736; SEQ ID NO: 737; SEQ ID NO: 738; SEQ ID NO: 739; SEQ ID NO: 740; SEQ ID NO: 741; SEQ ID NO: 742; SEQ ID NO: 743; SEQ ID NO: 744; SEQ ID NO: 745; SEQ ID NO: 746; SEQ ID NO: 747; SEQ ID NO: 748; SEQ ID NO: 749; SEQ ID NO: 750; SEQ ID NO: 751; SEQ ID NO: 752; SEQ ID NO: 753; SEQ ID NO: 754; SEQ ID NO: 755; SEQ ID NO: 756; SEQ ID NO: 757; SEQ ID NO: 758; SEQ ID NO: 759; SEQ ID NO: 760; SEQ ID NO: 761; SEQ ID NO: 762; SEQ ID NO: 763; SEQ ID NO: 764; SEQ ID NO: 765; SEQ ID NO: 766; SEQ ID NO: 767; SEQ ID NO: 768; SEQ ID NO: 769; SEQ ID NO: 770; SEQ ID NO: 771; SEQ ID NO: 772; SEQ ID NO: 773; SEQ ID NO: 774; SEQ ID NO: 775; SEQ ID NO: 776; SEQ ID NO: 777; SEQ ID NO: 778; SEQ ID NO: 779; SEQ ID NO: 780; SEQ ID NO: 781; SEQ ID NO: 782; SEQ ID NO: 783; SEQ ID NO: 784; SEQ ID NO: 785; SEQ ID NO: 786; SEQ ID NO: 787; SEQ ID NO: 788; SEQ ID NO: 789; SEQ ID NO: 790; SEQ ID NO: 791; SEQ ID NO: 792; SEQ ID NO: 793; SEQ ID NO: 794; SEQ ID NO: 795; SEQ ID NO: 796; SEQ ID NO: 797; SEQ ID NO: 798; SEQ ID NO: 799; SEQ ID NO: 800; SEQ ID NO: 801; SEQ ID NO: 802; SEQ ID NO: 803; SEQ ID NO: 804; SEQ ID NO: 805; SEQ ID NO: 806; SEQ ID NO: 807; SEQ ID NO: 808; SEQ ID NO: 809; SEQ ID NO: 810; SEQ ID NO: 811; SEQ ID NO: 812; SEQ ID NO: 813; SEQ ID NO: 814; SEQ ID NO: 815; SEQ ID NO: 816; SEQ ID NO: 817; SEQ ID NO: 818; SEQ ID NO: 819; SEQ ID NO: 820; SEQ ID NO: 821; SEQ ID NO: 822; SEQ ID NO: 823; SEQ ID NO: 824; SEQ ID NO: 825; SEQ ID NO: 826; SEQ ID NO: 827; SEQ ID NO: 828; SEQ ID NO: 829; SEQ ID NO: 830; SEQ ID NO: 831; SEQ ID NO: 832; SEQ ID NO: 833; SEQ ID NO: 834; SEQ ID NO: 835; SEQ ID NO: 836; SEQ ID NO: 837; SEQ ID NO: 838; SEQ ID NO: 839; SEQ ID NO: 840; SEQ ID NO: 841; SEQ ID NO: 842; SEQ ID NO: 843; SEQ ID NO: 844; SEQ ID NO: 845; SEQ ID NO: 846; SEQ ID NO: 847; SEQ ID NO: 848; SEQ ID NO: 849; SEQ ID NO: 850; SEQ ID NO: 851; SEQ ID NO: 852; SEQ ID NO: 853; SEQ ID NO: 854; SEQ ID NO: 855; SEQ ID NO: 856; SEQ ID NO: 857; SEQ ID NO: 858; SEQ ID NO: 859; SEQ ID NO: 860; SEQ ID NO: 861; SEQ ID NO: 862; SEQ ID NO: 863; SEQ ID NO: 864; SEQ ID NO: 865; SEQ ID NO: 866; SEQ ID NO: 867; SEQ ID NO: 868; SEQ ID NO: 869; SEQ ID NO: 870; SEQ ID NO: 871; SEQ ID NO: 872; SEQ ID NO: 873; SEQ ID NO: 874; SEQ ID NO: 875; SEQ ID NO: 876; SEQ ID NO: 877; SEQ ID NO: 878; SEQ ID NO: 879; SEQ ID NO: 880; SEQ ID NO: 881; SEQ ID NO: 882; SEQ ID NO: 883; SEQ ID NO: 884; SEQ ID NO: 885; SEQ ID NO: 886; SEQ ID NO: 887; SEQ ID NO: 888; SEQ ID NO: 889; SEQ ID NO: 890; SEQ ID NO: 891; SEQ ID NO: 892; SEQ ID NO: 893; SEQ ID NO: 894; SEQ ID NO: 895; SEQ ID NO: 896; SEQ ID NO: 897; SEQ ID NO: 898; SEQ ID NO: 899; SEQ ID NO: 900; SEQ ID NO: 901; SEQ ID NO: 902; SEQ ID NO: 903; SEQ ID NO: 904; SEQ ID NO: 905; SEQ ID NO: 906; SEQ ID NO: 907; SEQ ID NO: 908; SEQ ID NO: 909; SEQ ID NO: 910; SEQ ID NO: 911; SEQ ID NO: 912; SEQ ID NO: 913; SEQ ID NO: 914; SEQ ID NO: 915; SEQ ID NO: 916; SEQ ID NO: 917; SEQ ID NO: 918; SEQ ID NO: 919; SEQ ID NO: 920; SEQ ID NO: 921; SEQ ID NO: 922; SEQ ID NO: 923; SEQ ID NO: 924; SEQ ID NO: 925; SEQ ID NO: 926; SEQ ID NO: 927; SEQ ID NO: 928; SEQ ID NO: 929; SEQ ID NO: 930; SEQ ID NO: 931; SEQ ID NO: 932; SEQ ID NO: 933; SEQ ID NO: 934; SEQ ID NO: 935; SEQ ID NO: 936; SEQ ID NO: 937; SEQ ID NO: 938; SEQ ID NO: 939; SEQ ID NO: 940; SEQ ID NO: 941; SEQ ID NO: 942; SEQ ID NO: 943; SEQ ID NO: 944; SEQ ID NO: 945; SEQ ID NO: 946; SEQ ID NO: 947; SEQ ID NO: 948; SEQ ID NO: 949; SEQ ID NO: 950; SEQ ID NO: 951; SEQ ID NO: 952; SEQ ID NO: 953; SEQ ID NO: 954; SEQ ID NO: 955; SEQ ID NO: 956; SEQ ID NO: 957; SEQ ID NO: 958; SEQ ID NO: 959; SEQ ID NO: 960; SEQ ID NO: 961; SEQ ID NO: 962; SEQ ID NO: 963; SEQ ID NO: 964; SEQ ID NO: 965; SEQ ID NO: 966; SEQ ID NO: 967; SEQ ID NO: 968; SEQ ID NO: 969; SEQ ID NO: 970; SEQ ID NO: 971; SEQ ID NO: 972; SEQ ID NO: 973; SEQ ID NO: 974; SEQ ID NO: 975; SEQ ID NO: 976; SEQ ID NO: 977; SEQ ID NO: 978; SEQ ID NO: 979; SEQ ID NO: 980; SEQ ID NO: 981; SEQ ID NO: 982; SEQ ID NO: 983; SEQ ID NO: 984; SEQ ID NO: 985; SEQ ID NO: 986; SEQ ID NO: 987; SEQ ID NO: 988; SEQ ID NO: 989; SEQ ID NO: 990; SEQ ID NO: 991; SEQ ID NO: 992; SEQ ID NO: 993; SEQ ID NO: 994; SEQ ID NO: 995; SEQ ID NO: 996; SEQ ID NO: 997; SEQ ID NO: 998; SEQ ID NO: 999; SEQ ID NO: 1000; SEQ ID NO: 1001; SEQ ID NO: 1002; SEQ ID NO: 1003; SEQ ID NO: 1004; SEQ ID NO: 1005; SEQ ID NO: 1006; SEQ ID NO: 1007; SEQ ID NO: 1008; SEQ ID NO: 1009; SEQ ID NO: 1010; SEQ ID NO: 1011; SEQ ID NO: 1012; SEQ ID NO: 1013; SEQ ID NO: 1014; SEQ ID NO: 1015; SEQ ID NO: 1016; SEQ ID NO: 1017; SEQ ID NO: 1018; SEQ ID NO: 1019; SEQ ID NO: 1020; SEQ ID NO: 1021; SEQ ID NO: 1022; SEQ ID NO: 1023; SEQ ID NO: 1024; SEQ ID NO: 1025; SEQ ID NO: 1026; SEQ ID NO: 1027; SEQ ID NO: 1028; SEQ ID NO: 1029; SEQ ID NO: 1030; SEQ ID NO: 1031; SEQ ID NO: 1032; SEQ ID NO: 1033; SEQ ID NO: 1034; SEQ ID NO: 1035; SEQ ID NO: 1036; SEQ ID NO: 1037; SEQ ID NO: 1038; SEQ ID NO: 1039; SEQ ID NO: 1040; SEQ ID NO: 1041; SEQ ID NO: 1042; SEQ ID NO: 1043; SEQ ID NO: 1044; SEQ ID NO: 1045; SEQ ID NO: 1046; SEQ ID NO: 1047; SEQ ID NO: 1048; SEQ ID NO: 1049; SEQ ID NO: 1050; SEQ ID NO: 1051; SEQ ID NO: 1052; SEQ ID NO: 1053; SEQ ID NO: 1054; SEQ ID NO: 1055; SEQ ID NO: 1056; SEQ ID NO: 1057; SEQ ID NO: 1058; SEQ ID NO: 1059; SEQ ID NO: 1060; SEQ ID NO: 1061; SEQ ID NO: 1062; SEQ ID NO: 1063; SEQ ID NO: 1064; SEQ ID NO: 1065; SEQ ID NO: 1066; SEQ ID NO: 1067; SEQ ID NO: 1068; SEQ ID NO: 1069; SEQ ID NO: 1070; SEQ ID NO: 1071; SEQ ID NO: 1072; SEQ ID NO: 1073; SEQ ID NO: 1074; SEQ ID NO: 1075; SEQ ID NO: 1076; SEQ ID NO: 1077; SEQ ID NO: 1078; SEQ ID NO: 1079; SEQ ID NO: 1080; SEQ ID NO: 1081; SEQ ID NO: 1082; SEQ ID NO: 1083; SEQ ID NO: 1084; SEQ ID NO: 1085; SEQ ID NO: 1086; SEQ ID NO: 1087; SEQ ID NO: 1088; SEQ ID NO: 1089; SEQ ID NO: 1090; SEQ ID NO: 1091; SEQ ID NO: 1092; SEQ ID NO: 1093; SEQ ID NO: 1094; SEQ ID NO: 1095; SEQ ID NO: 1096; SEQ ID NO: 1097; SEQ ID NO: 1098; SEQ ID NO: 1099; SEQ ID NO: 1100; SEQ ID NO: 1101; SEQ ID NO: 1102; SEQ ID NO: 1103; SEQ ID NO: 1104; SEQ ID NO: 1105; SEQ ID NO: 1106; SEQ ID NO: 1107; SEQ ID NO: 1108; SEQ ID NO: 1109; SEQ ID NO: 1110; SEQ ID NO: 1111; SEQ ID NO: 1112; SEQ ID NO: 1113; SEQ ID NO: 1114; SEQ ID NO: 1115; SEQ ID NO: 1116; SEQ ID NO: 1117; SEQ ID NO: 1118; SEQ ID NO: 1119; SEQ ID NO: 1120; SEQ ID NO: 1121; SEQ ID NO: 1122; SEQ ID NO: 1123; SEQ ID NO: 1124; SEQ ID NO: 1125; SEQ ID NO: 1126; SEQ ID NO: 1127; SEQ ID NO: 1128; SEQ ID NO: 1129; SEQ ID NO: 1130; SEQ ID NO: 1131; SEQ ID NO: 1132; SEQ ID NO: 1133; SEQ ID NO: 1134; SEQ ID NO: 1135; SEQ ID NO: 1136; SEQ ID NO: 1137; SEQ ID NO: 1138; SEQ ID NO: 1139; SEQ ID NO: 1140; SEQ ID NO: 1141; SEQ ID NO: 1142; SEQ ID NO: 1143; SEQ ID NO: 1144; SEQ ID NO: 1145; SEQ ID NO: 1146; SEQ ID NO: 1147; SEQ ID NO: 1148; SEQ ID NO: 1149; SEQ ID NO: 1150; SEQ ID NO: 1151; SEQ ID NO: 1152; SEQ ID NO: 1153; SEQ ID NO: 1154; SEQ ID NO: 1155; SEQ ID NO: 1156; SEQ ID NO: 1157; SEQ ID NO: 1158; SEQ ID NO: 1159; SEQ ID NO: 1160; SEQ ID NO: 1161; SEQ ID NO: 1162; SEQ ID NO: 1163; SEQ ID NO: 1164; SEQ ID NO: 1165; SEQ ID NO: 1166; SEQ ID NO: 1167; SEQ ID NO: 1168; SEQ ID NO: 1169; SEQ ID NO: 1170; SEQ ID NO: 1171; SEQ ID NO: 1172; SEQ ID NO: 1173; SEQ ID NO: 1174; SEQ ID NO: 1175; SEQ ID NO: 1176; SEQ ID NO: 1177; SEQ ID NO: 1178; SEQ ID NO: 1179; SEQ ID NO: 1180; SEQ ID NO: 1181; SEQ ID NO: 1182; SEQ ID NO: 1183; SEQ ID NO: 1184; SEQ ID NO: 1185; SEQ ID NO: 1186; SEQ ID NO: 1187; SEQ ID NO: 1188; SEQ ID NO: 1189; SEQ ID NO: 1190; SEQ ID NO: 1191; SEQ ID NO: 1192; SEQ. ID NO: 1193; SEQ ID NO: 1194; SEQ ID NO: 1195; SEQ ID NO: 1196; SEQ ID NO: 119; SEQ ID NO: 1198; SEQ ID NO: 1199; SEQ ID NO: 1200; SEQ ID NO: 1201; SEQ ID NO: 1202; SEQ ID NO: 1203; SEQ ID NO: 1204; SEQ ID NO: 1205; SEQ ID NO: 1206; SEQ ID NO: 1207; SEQ ID NO: 1208; SEQ ID NO: 1209; SEQ ID NO: 1210; SEQ ID NO: ¹²¹I; SEQ ID NO: 1212; SEQ ID NO: 1213; SEQ ID NO: 1214; SEQ ID NO: 1215; SEQ ID NO: 1216; SEQ ID NO: 1217; SEQ ID NO: 1218; SEQ ID NO: 1219; SEQ ID NO: 1220; SEQ ID NO: 1221; SEQ ID NO: 1222; SEQ ID NO: 1223; SEQ ID NO: 1224; SEQ ID NO: 1225; SEQ ID NO: 1226; SEQ ID NO: 1227; SEQ ID NO: 1228; SEQ ID NO: 1229; SEQ ID NO: 1230; SEQ ID NO: 1231; SEQ ID NO: 1232; SEQ ID NO: 1233; SEQ ID NO: 1234; SEQ ID NO: 1235; SEQ ID NO: 1236; SEQ ID NO: 1237; SEQ ID NO: 1238; SEQ ID NO: 1239; SEQ ID NO: 1240; SEQ ID NO: 1241; SEQ ID NO: 1242; SEQ ID NO: 1243; SEQ ID NO: 1244; SEQ ID NO: 1245; SEQ ID NO: 1246; SEQ ID NO: 1247; SEQ ID NO: 1248; SEQ ID NO: 1249; SEQ ID NO: 1250; SEQ ID NO: 1251; SEQ ID NO: 1252; SEQ ID NO: 1253; SEQ ID NO: 1254; SEQ ID NO: 1255; SEQ ID NO: 1256; SEQ ID NO: 1257; SEQ ID NO: 1258; SEQ ID NO: 1259; SEQ ID NO: 1260; SEQ ID NO: 1261; SEQ ID NO: 1262; SEQ ID NO: 1263; SEQ ID NO: 1264; SEQ ID NO: 1265; SEQ ID NO: 1266; SEQ ID NO: 1267; SEQ ID NO: 1268; SEQ ID NO: 1269; SEQ ID NO: 1270; SEQ ID NO: 1271; SEQ ID NO: 1272; SEQ ID NO: 1273; SEQ ID NO: 1274; SEQ ID NO: 1275; SEQ ID NO: 1276; SEQ ID NO: 1277; SEQ ID NO: 1278; SEQ ID NO: 1279; SEQ ID NO: 1280; SEQ ID NO: 1281; SEQ ID NO: 1282; SEQ ID NO: 1283; SEQ ID NO: 1284; SEQ ID NO: 1285; SEQ ID NO: 1286; SEQ ID NO: 1287; SEQ ID NO: 1288; SEQ ID NO: 1289; SEQ ID NO: 1290; SEQ ID NO: 1291; SEQ ID NO: 1292; SEQ ID NO: 1293; SEQ ID NO: 1294; SEQ ID NO: 1295; SEQ ID NO: 1296; SEQ ID NO: 1297; SEQ ID NO: 1298; SEQ ID NO: 1299; SEQ ID NO: 1300; SEQ ID NO: 1301; SEQ ID NO: 1302; SEQ ID NO: 1303; SEQ ID NO: 1304; SEQ ID NO: 1305; SEQ ID NO: 1306; SEQ ID NO: 1307; SEQ ID NO: 1308; SEQ ID NO: 1309; SEQ ID NO: 1310; SEQ ID NO: ¹³¹I; SEQ ID NO: 1312; SEQ ID NO:1313; SEQ ID NO: 1314; SEQ ID NO: 1315; SEQ ID NO: 1316; SEQ ID NO: 1317; SEQ ID NO: 1318; SEQ ID NO: 1319; SEQ ID NO: 1320; SEQ ID NO: 1321; SEQ ID NO: 1322; SEQ ID NO: 1323; SEQ ID NO: 1324; SEQ ID NO: 1325; SEQ ID NO: 1326; SEQ ID NO: 1327; SEQ ID NO: 1328; SEQ ID NO: 1329; SEQ ID NO: 1330; SEQ ID NO: 1331; SEQ ID NO: 1332; SEQ ID NO: 1333; SEQ ID NO: 1334; SEQ ID NO: 1335; SEQ ID NO: 1336; SEQ ID NO: 1337; SEQ ID NO: 1338; SEQ ID NO: 1339; SEQ ID NO: 1340; SEQ ID NO: 1341; SEQ ID NO: 1342; SEQ ID NO: 1343; SEQ ID NO: 1344; SEQ ID NO: 1345; SEQ ID NO: 1346; SEQ ID NO: 1347; SEQ ID NO: 1348; SEQ ID NO: 1349; SEQ ID NO: 1350; SEQ ID NO: 1351; SEQ ID NO: 1352; SEQ ID NO: 1353; SEQ ID NO: 1354; SEQ ID NO: 1355; SEQ ID NO: 1356; SEQ ID NO: 1357; SEQ ID NO: 1358; SEQ ID NO: 1359; SEQ ID NO: 1360; SEQ ID NO: 1361; SEQ ID NO: 1362; SEQ ID NO: 1363; SEQ ID NO: 1364; SEQ ID NO: 1365; SEQ ID NO: 1366; SEQ ID NO: 1367; SEQ ID NO: 1368; SEQ ID NO: 1369; SEQ ID NO: 1370; SEQ ID NO: 1371; SEQ ID NO: 1372; SEQ ID NO: 1373; SEQ ID NO: 1374; SEQ ID NO: 1375; SEQ ID NO: 1376; SEQ ID NO: 1377; SEQ ID NO: 1378; SEQ ID NO: 1379; SEQ ID NO: 1380; SEQ ID NO: 1381; SEQ ID NO: 1382; SEQ ID NO: 1383; SEQ ID NO: 1384; SEQ ID NO: 1385; SEQ ID NO: 1386; SEQ ID NO: 1387; SEQ ID NO: 1388; SEQ ID NO: 1389; SEQ ID NO: 1390; SEQ ID NO: 1391; SEQ ID NO: 1392; SEQ ID NO: 1393; SEQ ID NO: 1394; SEQ ID NO: 1395; SEQ ID NO: 1396; SEQ ID NO: 1397; SEQ ID NO: 1398; SEQ ID NO: 1399; SEQ ID NO: 1400; SEQ ID NO: 1401; SEQ ID NO: 1402; SEQ ID NO: 1403; SEQ ID NO: 1404; SEQ ID NO: 1405; SEQ ID NO: 1406; SEQ ID NO: 1407; SEQ ID NO: 1408; SEQ ID NO: 1409; SEQ ID NO: 1410; SEQ ID NO: 1411; SEQ ID NO: 1412; SEQ ID NO: 1413; SEQ ID NO: 1414; SEQ ID NO: 1415; SEQ ID NO: 1416; SEQ ID NO: 1417; SEQ ID NO: 1418; SEQ ID NO: 1419; SEQ ID NO: 1420; SEQ ID NO: 1421; SEQ ID NO: 1422; SEQ ID NO: 1423; SEQ ID NO: 1424; SEQ ID NO: 1425; SEQ ID NO: 1426; SEQ ID NO: 1427; SEQ ID NO: 1428; SEQ ID NO: 1429; SEQ ID NO: 1430; SEQ ID NO: 1431; SEQ ID NO: 1432; SEQ ID NO: 1433; SEQ ID NO: 1434; SEQ ID NO: 1435; SEQ ID NO: 1436; SEQ ID NO: 1437; SEQ ID NO: 1438; SEQ ID NO: 1439; SEQ ID NO: 1440; SEQ ID NO: 1441; SEQ ID NO: 1442; SEQ ID NO: 1443; SEQ ID NO: 1444; SEQ ID NO: 1445; SEQ ID NO: 1446; SEQ ID NO: 1447; SEQ ID NO: 1448; SEQ ID NO: 1449; SEQ ID NO: 1450; SEQ ID NO: 1451; SEQ ID NO: 1452; SEQ ID NO: 1453; SEQ ID NO: 1454; SEQ ID NO: 1455; SEQ ID NO: 1456; SEQ ID NO: 1457; SEQ ID NO: 1458; SEQ ID NO: 1459; SEQ ID NO: 1460; SEQ ID NO: 1461; SEQ ID NO: 1462; SEQ ID NO: 1463; SEQ ID NO: 1464; SEQ ID NO: 1465; SEQ ID NO: 1466; SEQ ID NO: 1467; SEQ ID NO: 1468; SEQ ID NO: 1469; SEQ ID NO: 1470; SEQ ID NO: 1471; SEQ ID NO: 1472; SEQ ID NO: 1473; SEQ ID NO: 1474; SEQ ID NO: 1475; SEQ ID NO: 1476; SEQ ID NO: 1477; SEQ ID NO: 1478;-SEQ ID NO: 1479; SEQ ID NO: 1480; SEQ ID NO: 1481; SEQ ID NO: 1482; SEQ ID NO: 1483; SEQ ID NO: 1484; SEQ ID NO: 1485; SEQ ID NO: 1486; SEQ ID NO: 1487; SEQ ID NO: 1488; SEQ ID NO: 1489; SEQ ID NO: 1490; SEQ ID NO: 1491; SEQ ID NO: 1492; SEQ ID NO: 1493; SEQ ID NO: 1494; SEQ ID NO: 1495; SEQ ID NO: 1496; SEQ ID NO: 1497; SEQ ID NO: 1498; SEQ ID NO: 1499; SEQ ID NO: 1500; SEQ ID NO: 1501; SEQ ID NO: 1502; SEQ ID NO: 1503; SEQ ID NO: 1504; SEQ ID NO: 1505; SEQ ID NO: 1506; SEQ ID NO: 1507; SEQ ID NO: 1508; SEQ ID NO: 1509; SEQ ID NO: 1510; SEQ ID NO: 1511; SEQ ID NO: 1512; SEQ ID NO: 1513; SEQ ID NO: 1514; SEQ ID NO: 9537; SEQ ID NO: 9538; SEQ ID NO: 9539; SEQ ID NO: 9540; SEQ ID NO: 9541; SEQ ID NO: 9542; SEQ ID NO: 9543; SEQ ID NO: 9544; SEQ ID NO: 9545; SEQ ID NO: 9546; SEQ ID NO: 9547; SEQ ID NO: 9548; SEQ ID NO: 9549; SEQ ID NO: 9550; SEQ ID NO: 9551; SEQ ID NO: 9552; SEQ ID NO: 9553; SEQ ID NO: 9554; SEQ ID NO: 9555; SEQ ID NO: 9556; SEQ ID NO: 9557; SEQ ID NO: 9558; SEQ ID NO: 9559; SEQ ID NO: 9560; SEQ ID NO: 9561; SEQ ID NO: 9562; SEQ ID NO: 9563; SEQ ID NO: 9564; SEQ ID NO: 9565; SEQ ID NO: 9566; SEQ ID NO: 9567; SEQ ID NO: 9568; SEQ ID NO: 9569; SEQ ID NO: 9570; SEQ ID NO: 9571; SEQ ID NO: 9572; SEQ ID NO: 9573; SEQ ID NO: 9574; SEQ ID NO: 9575; SEQ ID NO: 9576; SEQ ID NO: 9577; SEQ ID NO: 9578; SEQ ID NO: 9579; SEQ ID NO: 9580; SEQ ID NO: 9581; SEQ ID NO: 9582; SEQ ID NO: 9583; SEQ ID NO: 9584; SEQ ID NO: 9585; SEQ ID NO: 9586; SEQ ID NO: 9587; SEQ ID NO: 9588; SEQ ID NO: 9589; SEQ ID NO: 9590 and SEQ ID NO:9591.

In another embodiment, the H. pylori cell envelope polypeptide or a fragment thereof is an H. pylori inner membrane polypeptide or a fragment thereof involved in transport encoded by a nucleic acid having a nucleotide sequence selected from the group consisting of SEQ ID NO: 511; SEQ ID NO: 512; SEQ ID NO: 513; SEQ ID NO: 514; SEQ ID NO:515; SEQ ID NO:516; SEQ ID NO:517; SEQ ID NO:518; SEQ ID NO: 519; SEQ ID NO: 520; SEQ ID NO: 521; SEQ ID NO: 522; SEQ ID NO: 523; SEQ ID NO: 524; SEQ ID NO: 525; SEQ ID NO: 526; SEQ ID NO: 527; SEQ ID NO: 528; SEQ ID NO: 529; SEQ ID NO: 530; SEQ ID NO: 531; SEQ ID NO: 532; SEQ ID NO: 533; SEQ ID NO: 534; SEQ ID NO: 535; SEQ ID NO: 536; SEQ ID NO: 537; SEQ ID NO: 538; SEQ ID NO: 539; SEQ ID NO: 540; SEQ ID NO: 541; SEQ ID NO: 542; SEQ ID NO: 543; SEQ ID NO: 544; SEQ ID NO: 545; SEQ ID NO: 546; SEQ ID NO: 547; SEQ ID NO: 548; SEQ ID NO: 549; SEQ ID NO: 550; SEQ ID NO: 551; SEQ ID NO: 552; SEQ ID NO: 553; SEQ ID NO: 554; SEQ ID NO: 555; SEQ ID NO: 556; SEQ ID NO: 557; SEQ ID NO: 558; SEQ ID NO: 559; SEQ ID NO: 560; SEQ ID NO: 561; SEQ ID NO: 562; SEQ ID NO: 563; SEQ ID NO: 564; SEQ ID NO: 565; SEQ ID NO: 566; SEQ ID NO: 567; SEQ ID NO: 568; SEQ ID NO: 569; SEQ ID NO: 570; SEQ ID NO: 571; SEQ ID NO: 572; SEQ ID NO: 573; SEQ ID NO: 574; SEQ ID NO: 575; SEQ ID NO: 576; SEQ ID NO: 577; SEQ ID NO: 578; SEQ ID NO: 579; SEQ ID NO: 580; SEQ ID NO: 581; SEQ ID NO: 582; SEQ ID NO: 583; SEQ ID NO: 584; SEQ ID NO: 585; SEQ ID NO: 586; SEQ ID NO: 587; SEQ ID NO: 588; SEQ ID NO: 589; SEQ ID NO: 590; SEQ ID NO: 591; SEQ ID NO: 592; SEQ ID NO: 593; SEQ ID NO: 594; SEQ ID NO: 595; SEQ ID NO: 596; SEQ ID NO: 597; SEQ ID NO: 598; SEQ ID NO: 599; SEQ ID NO: 600; SEQ ID NO: 601; SEQ ID NO: 602; SEQ ID NO: 603; SEQ ID NO: 604; SEQ ID NO: 605; SEQ ID NO: 606; SEQ ID NO: 607; SEQ ID NO: 608; SEQ ID NO: 609; SEQ ID NO: 610; SEQ ID NO: 611; SEQ ID NO: 612; SEQ ID NO: 613; SEQ ID NO: 614; SEQ ID NO: 615; SEQ ID NO: 616; SEQ ID NO: 617; SEQ ID NO: 618; SEQ ID NO: 619; SEQ ID NO: 620; SEQ ID NO: 621; SEQ ID NO: 622; SEQ ID NO: 623; SEQ ID NO: 624; SEQ ID NO: 625; SEQ ID NO: 626; SEQ ID NO: 627; SEQ ID NO: 628; SEQ ID NO: 629; SEQ ID NO: 630; SEQ ID NO: 631; SEQ ID NO: 632; SEQ ID NO: 633; SEQ ID NO: 634; SEQ ID NO: 635; SEQ ID NO: 636; SEQ ID NO: 637; SEQ ID NO: 638; SEQ ID NO: 639; SEQ ID NO: 640; SEQ ID NO: 641; SEQ ID NO: 642; SEQ ID NO: 643; SEQ ID NO: 644; SEQ ID NO: 645; SEQ ID NO: 646; SEQ ID NO: 647; SEQ ID NO: 648; SEQ ID NO: 649; SEQ ID NO: 650; SEQ ID NO: 651; SEQ ID NO: 652; SEQ ID NO: 653; SEQ ID NO: 654; SEQ ID NO: 655; SEQ ID NO: 656; SEQ ID NO: 657; SEQ ID NO: 658; SEQ ID NO: 659; SEQ ID NO: 660; SEQ ID NO: 661; SEQ ID NO: 662; SEQ ID NO: 663; SEQ ID NO: 664; SEQ ID NO: 665; SEQ ID NO: 666; SEQ ID NO: 667; SEQ ID NO: 668; SEQ ID NO: 669; SEQ ID NO: 670; SEQ ID NO: 671; SEQ ID NO: 672; SEQ ID NO: 673; SEQ ID NO: 674; SEQ ID NO: 675; SEQ ID NO: 676; SEQ ID NO: 677; SEQ ID NO: 678; SEQ ID NO: 679; SEQ ID NO: 680; SEQ ID NO: 681; SEQ ID NO: 682; SEQ ID NO: 682; SEQ ID NO: 683; SEQ ID NO: 684; SEQ ID NO: 685; SEQ ID NO: 686; SEQ ID NO: 687; SEQ ID NO: 688; SEQ ID NO: 689; SEQ ID NO: 690; SEQ ID NO: 691; SEQ ID NO: 692; SEQ ID NO: 693; SEQ ID NO: 694; SEQ ID NO: 695; SEQ ID NO: 696; SEQ ID NO: 697; SEQ ID NO: 698; SEQ ID NO: 699; SEQ ID NO: 700; SEQ ID NO: 701; SEQ ID NO: 702; SEQ ID NO: 703; SEQ ID NO: 704; SEQ ID NO: 705; SEQ ID NO: 706; SEQ ID NO: 707; SEQ ID NO: 708; SEQ ID NO: 709; SEQ ID NO: 710; SEQ ID NO: 711; SEQ ID NO: 712; SEQ ID NO: 713; SEQ ID NO: 714; SEQ ID NO: 715; SEQ ID NO: 716; SEQ ID NO: 717; SEQ ID NO: 718; SEQ ID NO: 719; SEQ ID NO: 720; SEQ ID NO: 721; SEQ ID NO: 722; SEQ ID NO: 723; SEQ ID NO: 724; SEQ ID NO: 725; SEQ ID NO: 726; SEQ ID NO: 727; SEQ ID NO: 728; SEQ ID NO: 729; SEQ ID NO: 730; SEQ ID NO: 731; SEQ ID NO: 732; SEQ ID NO: 733; SEQ ID NO: 734; SEQ ID NO: 735; SEQ ID NO: 736; SEQ ID NO: 737; SEQ ID NO: 738; SEQ ID NO: 739; SEQ ID NO: 740; SEQ ID NO: 741; SEQ ID NO: 742; SEQ ID NO: 743; SEQ ID NO: 744; SEQ ID NO: 745; SEQ ID NO: 746; SEQ ID NO: 747; SEQ ID NO: 748; SEQ ID NO: 749; SEQ ID NO: 750; SEQ ID NO: 751; SEQ ID NO: 752; SEQ ID NO: 753; SEQ ID NO: 754; SEQ ID NO: 755; SEQ ID NO: 756; SEQ ID NO: 757; SEQ ID NO: 758; SEQ ID NO: 759; SEQ ID NO: 760; SEQ ID NO: 761; SEQ ID NO: 762; SEQ ID NO: 763; SEQ ID NO: 764; SEQ ID NO: 765; SEQ ID NO: 766; SEQ ID NO: 767; SEQ ID NO: 768; SEQ ID NO: 769; SEQ ID NO: 770; SEQ ID NO: 771; SEQ ID NO: 772; SEQ ID NO: 773; SEQ ID NO: 774; SEQ ID NO: 775; SEQ ID NO: 776; SEQ ID NO: 777; SEQ ID NO: 778; SEQ ID NO: 779; SEQ ID NO: 780; SEQ ID NO: 781; SEQ ID NO: 782; SEQ ID NO: 783; SEQ ID NO: 784; SEQ ID NO: 785; SEQ ID NO: 786; SEQ ID NO: 787; SEQ ID NO: 788; SEQ ID NO: 789; SEQ ID NO: 790; SEQ ID NO: 791; SEQ ID NO: 792; SEQ ID NO: 793; SEQ ID NO: 794; SEQ ID NO: 795; SEQ ID NO: 796; SEQ ID NO: 797; SEQ ID NO: 798; SEQ ID NO: 799; SEQ ID NO: 800; SEQ ID NO: 801; SEQ ID NO: 802; SEQ ID NO: 9537; SEQ ID NO: 9538; SEQ ID NO: 9539; SEQ ID NO: 9540; SEQ ID NO: 9541; SEQ ID NO: 9542; SEQ ID NO: 9543; SEQ ID NO: 9544; SEQ ID NO: 9545; SEQ ID NO: 9546; SEQ ID NO: 9547; SEQ ID NO: 9548; SEQ ID NO: 9549; SEQ ID NO: 9550; SEQ ID NO: 9551; SEQ ID NO: 9552; SEQ ID NO: 9553 and SEQ ID NO: 9554.

In another embodiment, the H. pylori cell envelope polypeptide or a fragment thereof is an H. pylori inner membrane polypeptide or a fragment thereof involved in amino acid metabolism and transport encoded by a nucleic acid having a nucleotide sequence selected from the group consisting of SEQ ID NO: 683; SEQ ID NO: 684; SEQ ID NO: 685; SEQ ID NO: 686; SEQ ID NO: 687; SEQ ID NO: 688; SEQ ID NO: 689; SEQ ID NO: 690; SEQ ID NO: 691; SEQ ID NO: 692; SEQ ID NO: 693; SEQ ID NO: 694; SEQ ID NO: 695; SEQ ID NO: 696; SEQ ID NO: 697; SEQ ID NO: 698; SEQ ID NO: 699; SEQ ID NO: 700; SEQ ID NO: 701; SEQ ID NO: 702; SEQ ID NO: 703; SEQ ID NO: 704; SEQ ID NO: 705; SEQ ID NO: 706; SEQ ID NO: 707; SEQ ID NO: 708; SEQ ID NO: 709; SEQ ID NO: 710; SEQ ID NO: 711; SEQ ID NO: 712; SEQ ID NO: 713; SEQ ID NO: 714; SEQ ID NO: 715; SEQ ID NO: 716; SEQ ID NO: 717; SEQ ID NO: 718; SEQ ID NO: 719; SEQ ID NO: 720; SEQ ID NO: 9547 and SEQ ID NO: 9548.

In another embodiment, the H. pylori cell envelope polypeptide or a fragment thereof is an H. pylori inner membrane polypeptide or a fragment thereof involved in nucleotide, lipid, or cofactor metabolism and transport encoded by a nucleic acid having a nucleotide sequence selected from the group consisting of SEQ ID NO: 721; SEQ ID NO: 722; SEQ ID NO: 723; SEQ ID NO: 724; SEQ ID NO: 725; SEQ ID NO: 726; SEQ ID NO: 727; SEQ ID NO: 728; SEQ ID NO: 729; SEQ ID NO: 730; SEQ ID NO: 731; SEQ ID NO: 732; SEQ ID NO: 733; SEQ ID NO: 734; SEQ ID NO: 735; SEQ ID NO: 736; SEQ ID NO: 737; SEQ ID NO: 738; SEQ ID NO: 739; SEQ ID NO: 740; SEQ ID NO: 741; SEQ ID NO: 742; SEQ ID NO: 743; SEQ ID NO: 744; SEQ ID NO: 745; SEQ ID NO: 746; SEQ ID NO: 747; SEQ ID NO: 748; SEQ ID NO: 749; SEQ ID NO: 750; SEQ ID NO: 751; SEQ ID NO: 752; SEQ ID NO: 753; SEQ ID NO: 754; SEQ ID NO: 755; SEQ ID NO: 756; SEQ ID NO: 757; SEQ ID NO: 758; SEQ ID NO: 759; SEQ ID NO: 760; SEQ ID NO: 761; SEQ ID NO: 762; SEQ ID NO: 763; SEQ ID NO: 764 and SEQ ID NO: 9549.

In another embodiment, the H. pylori cell envelope polypeptide or a fragment thereof is an H. pylori inner membrane polypeptide or a fragment thereof involved in inorganic ion transport encoded by a nucleic acid having a nucleotide sequence selected from the group consisting of SEQ ID NO: 765; SEQ ID NO: 766; SEQ ID NO: 767; SEQ ID NO: 768; SEQ ID NO: 769; SEQ ID NO: 770; SEQ ID NO: 771; SEQ ID NO: 772; SEQ ID NO: 773; SEQ ID NO: 774; SEQ ID NO: 775; SEQ ID NO: 776; SEQ ID NO: 777; SEQ ID NO: 778; SEQ ID NO: 779; SEQ ID NO: 780; SEQ ID NO: 781; SEQ ID NO: 782; SEQ ID NO: 783; SEQ ID NO: 784; SEQ ID NO: 785; SEQ ID NO: 786; SEQ ID NO: 787; SEQ ID NO: 788; SEQ ID NO: 789; SEQ ID NO: 790; SEQ ID NO: 791; SEQ ID NO: 792; SEQ ID NO: 793; SEQ ID NO: 794; SEQ ID NO: 795; SEQ ID NO: 796; SEQ ID NO: 797; SEQ ID NO: 798; SEQ ID NO: 799; SEQ ID NO: 9550; SEQ ID NO: 9551; SEQ ID NO: 9552; SEQ ID NO: 9553 and SEQ ID NO: 9554.

In another embodiment, the H. pylori cell envelope polypeptide or a fragment thereof is an H. pylori inner membrane polypeptide or a fragment thereof involved in carbohydrate metabolism and transport encoded by a nucleic acid having a nucleotide sequence selected from the group consisting of SEQ ID NO: 800; SEQ ID NO: 801; SEQ ID NO: 802.

In another embodiment, the H. pylori cell envelope polypeptide or a fragment thereof is an H. pylori inner membrane polypeptide or a fragment thereof involved in outer membrane and cell wall formation encoded by a nucleic acid having a nucleotide sequence selected from the group consisting of SEQ ID NO: 803; SEQ ID NO: 804; SEQ ID NO: 805; SEQ ID NO: 806; SEQ ID NO: 807; SEQ ID NO: 808; SEQ ID NO: 809; SEQ ID NO: 810; SEQ ID NO: 811; SEQ ID NO: 812; SEQ ID NO: 813; SEQ ID NO: 814; SEQ ID NO: 815; SEQ ID NO: 816; SEQ ID NO: 817; SEQ ID NO: 818; SEQ ID NO: 819; SEQ ID NO: 820; SEQ ID NO: 821; SEQ ID NO: 822; SEQ ID NO: 823; SEQ ID NO: 824; SEQ ID NO: 825; SEQ ID NO: 826; SEQ ID NO: 827; SEQ ID NO: 828; SEQ ID NO: 829; SEQ ID NO: 830; SEQ ID NO: 831; SEQ ID NO: 832; SEQ ID NO: 833; SEQ ID NO: 834; SEQ ID NO: 835; SEQ ID NO: 836; SEQ ID NO: 837; SEQ ID NO: 838; SEQ ID NO: 839; SEQ ID NO: 840; SEQ ID NO: 841; SEQ ID NO: 842; SEQ ID NO: 843; SEQ ID NO: 844; SEQ ID NO: 845; SEQ ID NO: 846; SEQ ID NO: 847; SEQ ID NO: 848; SEQ ID NO: 9555; SEQ ID NO: 9556; SEQ ID NO: 9557; SEQ ID NO: 9558; SEQ ID NO: 9559 and SEQ ID NO: 9560.

In another embodiment, the H. pylori cell envelope polypeptide or a fragment thereof is an H. pylori inner membrane polypeptide or a fragment thereof involved in energy conversion encoded by a nucleic acid having a nucleotide sequence selected from the group consisting of SEQ ID NO: 849; SEQ ID NO: 850; SEQ ID NO: 851; SEQ ID NO: 852; SEQ ID NO: 853; SEQ ID NO: 854; SEQ ID NO: 855; SEQ ID NO: 856; SEQ ID NO: 857; SEQ ID NO: 858; SEQ ID NO: 859; SEQ ID NO: 860; SEQ ID NO: 861; SEQ ID NO: 862; SEQ ID NO: 863; SEQ ID NO: 864; SEQ ID NO: 865; SEQ ID NO: 866; SEQ ID NO: 867; SEQ ID NO: 868; SEQ ID NO: 869; SEQ ID NO: 870; SEQ ID NO: 871; SEQ ID NO: 872; SEQ ID NO: 873; SEQ ID NO: 874; SEQ ID NO: 875; SEQ ID NO: 876; SEQ ID NO: 877; SEQ ID NO: 878; SEQ ID NO: 879; SEQ ID NO: 880; SEQ ID NO: 881; SEQ ID NO: 882; SEQ ID NO: 883; SEQ ID NO: 884; SEQ ID NO: 885; SEQ ID NO: 886; SEQ ID NO: 887; SEQ ID NO: 888; SEQ ID NO: 889; SEQ ID NO: 890; SEQ ID NO: 891; SEQ ID NO: 892; SEQ ID NO: 893; SEQ ID NO: 894; SEQ ID NO: 895; SEQ ID NO: 896; SEQ ID NO: 897; SEQ ID NO: 898; SEQ ID NO: 899; SEQ ID NO: 900; SEQ ID NO: 901; SEQ ID NO: 902; SEQ ID NO: 903; SEQ ID NO: 904; SEQ ID NO: 905; SEQ ID NO: 906; SEQ ID NO: 907; SEQ ID NO: 908; SEQ ID NO: 909; SEQ ID NO: 910; SEQ ID NO: 911; SEQ ID NO: 912; SEQ ID NO: 913; SEQ ID NO: 914; SEQ ID NO: 915; SEQ ID NO: 916; SEQ ID NO: 917; SEQ ID NO: 918; SEQ ID NO: 919; SEQ ID NO: 920; SEQ ID NO: 921; SEQ ID NO: 922; SEQ ID NO: 923; SEQ ID NO: 924; SEQ ID NO: 925; SEQ ID NO: 926; SEQ ID NO: 927; SEQ ID NO: 928; SEQ ID NO: 929; SEQ ID NO: 930; SEQ ID NO: 931; SEQ ID NO: 932; SEQ ID NO: 933; SEQ ID NO: 934; SEQ ID NO: 935; SEQ ID NO: 936; SEQ ID NO: 937; SEQ ID NO: 938; SEQ ID NO: 939; SEQ ID NO: 940; SEQ ID NO: 941; SEQ ID NO: 942; SEQ ID NO: 943; SEQ ID NO: 944; SEQ ID NO: 945; SEQ ID NO: 946; SEQ ID NO: 947; SEQ ID NO: 948; SEQ ID NO: 949; SEQ ID NO: 950; SEQ ID NO: 951; SEQ ID NO: 952; SEQ ID NO: 953; SEQ ID NO: 954; SEQ ID NO: 955; SEQ ID NO: 956; SEQ ID NO: 957; SEQ ID NO: 958; SEQ ID NO: 959; SEQ ID NO: 960; SEQ ID NO: 961; SEQ ID NO: 962; SEQ ID NO: 963; SEQ ID NO: 964; SEQ ID NO: 965; SEQ ID NO: 966; SEQ ID NO: 967; SEQ ID NO: 968; SEQ ID NO: 969; SEQ ID NO: 970; SEQ ID NO: 971; SEQ ID NO: 972; SEQ ID NO: 973; SEQ ID NO: 974; SEQ ID NO: 975; SEQ ID NO: 976; SEQ ID NO: 977; SEQ ID NO: 978; SEQ ID NO: 979; SEQ ID NO: 980; SEQ ID NO: 981; SEQ ID NO: 982; SEQ ID NO: 983; SEQ ID NO: 984; SEQ ID NO: 985; SEQ ID NO: 986; SEQ ID NO: 987; SEQ ID NO: 988; SEQ ID NO: 989; SEQ ID NO: 990; SEQ ID NO: 991; SEQ ID NO: 992; SEQ ID NO: 993; SEQ ID NO: 994; SEQ ID NO: 995; SEQ ID NO: 9561; SEQ ID NO: 9562; SEQ ID NO: 9563; SEQ ID NO: 9564 and SEQ ID NO: 9565.

In another embodiment, the H. pylori cell envelope polypeptide or a fragment thereof is an H. pylori inner membrane polypeptide or a fragment thereof involved in secretion and adhesion encoded by a nucleic acid having a nucleotide sequence selected from the group consisting of SEQ ID NO: 996; SEQ ID NO: 997; SEQ ID NO: 998; SEQ ID NO: 999; SEQ ID NO: 1000; SEQ ID NO: 1001; SEQ ID NO: 1002; SEQ ID NO: 1003; SEQ ID NO: 1004; SEQ ID NO: 1005; SEQ ID NO: 1006; SEQ ID NO: 1007 and SEQ ID NO: 9566.

In another embodiment, the H. pylori cell envelope polypeptide or a fragment thereof is an H. pylori inner membrane polypeptide or a fragment thereof involved in regulation encoded by a nucleic acid having a nucleotide sequence selected from the group consisting of SEQ ID NO: 1008; SEQ ID NO: 1009; SEQ ID NO: 1010; SEQ ID NO: 1011; SEQ ID NO: 1012; SEQ ID NO 1013; SEQ ID NO: 1014; SEQ ID NO: 1015; SEQ ID NO: 1016; SEQ ID NO: 1017; SEQ ID NO: 1018; SEQ ID NO: 1019; SEQ ID NO: 1020; SEQ ID NO: 1021; SEQ ID NO: 1022; SEQ ID NO: 1023; SEQ ID NO: 1024; SEQ ID NO: 1025; SEQ ID NO: 1026 and SEQ ID NO: 1027.

In another embodiment, the H. pylori cell envelope polypeptide or a fragment thereof is an H. pylori inner membrane chaperone polypeptide or a fragment thereof encoded by a nucleic acid having a nucleotide sequence selected from the group consisting of SEQ ID NO: 1028; SEQ ID NO: 1029; SEQ ID NO: 1030; SEQ ID NO: 1031 and SEQ ID NO: 9567.

In another embodiment, the H. pylori cell envelope polypeptide or a fragment thereof is an H. pylori inner membrane polypeptide or a fragment thereof involved in cell division encoded by a nucleic acid having a nucleotide sequence selected from the group consisting of SEQ ID NO: 1032; SEQ ID NO: 1033; SEQ ID NO: 1034; SEQ ID NO: 1035; SEQ ID NO: 1036; SEQ ID NO: 1037; SEQ ID NO: 1038; SEQ ID NO: 1039; SEQ ID NO: 1040; SEQ ID NO: 1041; SEQ ID NO: 1042; SEQ ID NO: 1043; SEQ ID NO: 1044; SEQ ID NO: 1045; SEQ ID NO: 1046; SEQ ID NO: 1047; SEQ ID NO: 1048; SEQ ID NO: 1049; SEQ ID NO: 1050; SEQ ID NO: 1051; SEQ ID NO: 1052; SEQ ID NO: 9568 and SEQ ID NO: 9569.

In another embodiment, the H. pylori cell envelope polypeptide or a fragment thereof is an H. pylori inner membrane polypeptide or a fragment thereof involved in motility encoded by a nucleic acid having a nucleotide sequence selected from the group consisting of SEQ ID NO: 1053 and SEQ ID NO: 1054.

Particularly preferred is an isolated H. pylori cytoplasmic polypeptide or a fragment thereof, wherein the polypeptide has an amino acid sequence selected from the group consisting of SEQ ID NO: 6338; SEQ ID NO: 6339; SEQ ID NO: 6340; SEQ ID NO: 6341; SEQ ID NO: 6342; SEQ ID NO: 6343; SEQ ID NO: 6344; SEQ ID NO: 6345; SEQ ID NO: 6346; SEQ ID NO: 6347; SEQ ID NO: 6348; SEQ ID NO: 6349; SEQ ID NO: 6350; SEQ ID NO: 6351; SEQ ID NO: 6352; SEQ ID NO: 6353; SEQ ID NO: 6354; SEQ ID NO: 6355; SEQ ID NO: 6356; SEQ ID NO: 6357; SEQ ID NO: 6358; SEQ ID NO: 6359; SEQ ID NO: 6360; SEQ ID NO: 6361; SEQ ID NO: 6362; SEQ ID NO: 6363; SEQ ID NO: 6364; SEQ ID NO: 6365; SEQ ID NO: 6366; SEQ ID NO: 6367; SEQ ID NO: 6368; SEQ ID NO: 6369; SEQ ID NO: 6370; SEQ ID NO: 6371; SEQ ID NO: 6372; SEQ ID NO: 6373; SEQ ID NO: 6374; SEQ ID NO: 6375; SEQ ID NO: 6376; SEQ ID NO: 6377; SEQ ID NO: 6378; SEQ ID NO: 6379; SEQ ID NO: 6380; SEQ ID NO: 6381; SEQ ID NO: 6382; SEQ ID NO: 6383; SEQ ID NO: 6384; SEQ ID NO: 6385; SEQ ID NO: 6386; SEQ ID NO: 6387; SEQ ID NO: 6388; SEQ ID NO: 6389; SEQ ID NO: 6390; SEQ ID NO: 6391; SEQ ID NO: 6392; SEQ ID NO: 6393; SEQ ID NO: 6394; SEQ ID NO: 6395; SEQ ID NO: 6396; SEQ ID NO: 6397; SEQ ID NO: 6398; SEQ ID NO: 6399; SEQ ID NO: 6400; SEQ ID NO: 6401; SEQ ID NC. 6402; SEQ ID NO: 6403; SEQ ID NO: 6404; SEQ ID NO: 6405; SEQ ID NO: 6406; SEQ ID NO: 6407; SEQ ID NO: 6408; SEQ ID NO: 6409; SEQ ID NO: 6410; SEQ ID NO: 6411; SEQ ID NO: 6412; SEQ ID NO: 6413; SEQ ID NO: 6414; SEQ ID NO: 6415; SEQ ID NO: 6416; SEQ ID NO: 6417; SEQ ID NO: 6418; SEQ ID NO: 6419; SEQ ID NO: 6420; SEQ ID NO: 6421; SEQ ID NO: 6422; SEQ ID NO: 6423; SEQ ID NO: 6424; SEQ ID NO: 6425; SEQ ID NO: 6426; SEQ ID NO: 6427; SEQ ID NO: 6428; SEQ ID NO: 6429; SEQ ID NO: 6430; SEQ ID NO: 6431; SEQ ID NO: 6432; SEQ ID NO: 6433; SEQ ID NO: 6434; SEQ ID NO: 6435; SEQ ID NO: 6436; SEQ ID NO: 6437; SEQ ID NO: 6438; SEQ ID NO: 6439; SEQ ID NO: 6440; SEQ ID NO: 6441; SEQ ID NO: 6442; SEQ ID NO: 6443; SEQ ID NO: 6444; SEQ ID NO: 6445; SEQ ID NO: 6446; SEQ ID NO: 6447; SEQ ID NO: 6448; SEQ ID NO: 6449; SEQ ID NO: 6450; SEQ ID NO: 6451; SEQ ID NO: 6452; SEQ ID NO: 6453; SEQ ID NO: 6454; SEQ ID NO: 6455; SEQ ID NO: 6456; SEQ ID NO: 6457; SEQ ID NO: 6458; SEQ ID NO: 6459; SEQ ID NO: 6460; SEQ ID NO: 6461; SEQ ID NO: 6462; SEQ ID NO: 6463; SEQ ID NO: 6464; SEQ ID NO: 6465; SEQ ID NO: 6466; SEQ ID NO: 6467; SEQ ID NO: 6468; SEQ ID NO: 6469; SEQ ID NO: 6470; SEQ ID NO: 6471; SEQ ID NO: 6472; SEQ ID NO: 6473; SEQ ID NO: 6474; SEQ ID NO: 6475; SEQ ID NO: 6476; SEQ ID NO: 6477; SEQ ID NO: 6478; SEQ ID NO: 6479; SEQ ID NO: 6480; SEQ ID NO: 6481; SEQ ID NO: 6482; SEQ ID NO: 6483; SEQ ID NO: 6484; SEQ ID NO: 6485; SEQ ID NO: 6486; SEQ ID NO: 6487; SEQ ID NO: 6488; SEQ ID NO: 6489; SEQ ID NO: 6490; SEQ ID NO: 6491; SEQ ID NO: 6492; SEQ ID NO: 6493; SEQ ID NO: 6494; SEQ ID NO: 6495; SEQ ID NO: 6496; SEQ ID NO: 6497; SEQ ID NO: 6498; SEQ ID NO: 6499; SEQ ID NO: 6500; SEQ ID NO: 6501; SEQ ID NO: 6502; SEQ ID NO: 6503; SEQ ID NO: 6504; SEQ ID NO: 6505; SEQ ID NO: 6506; SEQ ID NO: 6507; SEQ ID NO: 6508; SEQ ID NO: 6509; SEQ ID NO: 6510; SEQ ID NO: 6511; SEQ ID NO: 6512; SEQ ID NO: 6513; SEQ ID NO: 6514; SEQ ID NO: 6515; SEQ ID NO: 6516; SEQ ID NO: 6517; SEQ ID NO: 6518; SEQ ID NO: 6519; SEQ ID NO: 6520; SEQ ID NO: 6521; SEQ ID NO: 6522; SEQ ID NO: 6523; SEQ ID NO: 6524; SEQ ID NO: 6525; SEQ ID NO: 6526; SEQ ID NO: 6527; SEQ ID NO: 6528; SEQ ID NO: 6529; SEQ ID NO: 6530; SEQ ID NO: 6531; SEQ ID NO: 6532; SEQ ID NO: 6533; SEQ ID NO: 6534; SEQ ID NO: 6535; SEQ ID NO: 6536; SEQ ID NO: 6537; SEQ ID NO: 6538; SEQ ID NO: 6539; SEQ ID NO: 6540; SEQ ID NO: 6541; SEQ ID NO: 6542; SEQ ID NO: 6543; SEQ ID NO: 6544; SEQ ID NO: 6545; SEQ ID NO: 6546; SEQ ID NO: 6547; SEQ ID NO: 6548; SEQ ID NO: 6549; SEQ ID NO: 6550; SEQ ID NO: 6551; SEQ ID NO: 6552; SEQ ID NO: 6553; SEQ ID NO: 6554; SEQ ID NO: 6555; SEQ ID NO: 6556; SEQ ID NO: 6557; SEQ ID NO: 6558; SEQ ID NO: 6559; SEQ ID NO: 6560; SEQ ID NO: 6561; SEQ ID NO: 6562; SEQ ID NO: 6563; SEQ ID NO: 6564; SEQ ID NO: 6565; SEQ ID NO: 6566; SEQ ID NO: 6567; SEQ ID NO: 6568; SEQ ID NO: 6569; SEQ ID NO: 6570; SEQ ID NO: 6571; SEQ ID NO: 6572; SEQ ID NO: 6573; SEQ ID NO: 6574; SEQ ID NO: 6575; SEQ ID NO: 6576; SEQ ID NO: 6577; SEQ ID NO: 6578; SEQ ID NO: 6579; SEQ ID NO: 6580; SEQ ID NO: 6581; SEQ ID NO: 6582; SEQ ID NO: 6583; SEQ ID NO: 6584; SEQ ID NO: 6585; SEQ ID NO: 6586; SEQ ID NO: 6587; SEQ ID NO: 6588; SEQ ID NO: 6589; SEQ ID NO: 6590; SEQ ID NO: 6591; SEQ ID NO: 6592; SEQ ID NO: 6593; SEQ ID NO: 6594; SEQ ID NO: 6595; SEQ ID NO: 6596; SEQ ID NO: 6597; SEQ ID NO: 6598; SEQ ID NO: 6599; SEQ ID NO: 6600; SEQ ID NO: 6601; SEQ ID NO: 6602; SEQ ID NO: 6603; SEQ ID NO: 6604; SEQ ID NO: 6605; SEQ ID NO: 6606; SEQ ID NO: 6607; SEQ ID NO: 6608; SEQ ID NO: 6609; SEQ ID NO: 6610; SEQ ID NO: 6611; SEQ ID NO: 6612; SEQ ID NO: 6613; SEQ ID NO: 6614; SEQ ID NO: 6615; SEQ ID NO: 6616; SEQ ID NO: 6617; SEQ ID NO: 6618; SEQ ID NO: 6619; SEQ ID NO: 6620; SEQ ID NO: 6621; SEQ ID NO: 6622; SEQ ID NO: 6623; SEQ ID NO: 6624; SEQ ID NO: 6625; SEQ ID NO: 6626; SEQ ID NO: 6627; SEQ ID NO: 6628; SEQ ID NO: 6629; SEQ ID NO: 6630; SEQ ID NO: 6631; SEQ ID NO: 6632; SEQ ID NO: 6633; SEQ ID NO: 6634; SEQ ID NO: 6635; SEQ ID NO: 6636; SEQ ID NO: 6637; SEQ ID NO: 6638; SEQ ID NO: 6639; SEQ ID NO: 6640; SEQ ID NO: 6641; SEQ ID NO: 6642; SEQ ID NO: 6643; SEQ ID NO: 6644; SEQ ID NO: 6645; SEQ ID NO: 6646; SEQ ID NO: 6647; SEQ ID NO: 6648; SEQ ID NO: 6649; SEQ ID NO: 6650; SEQ ID NO: 6651; SEQ ID NO: 6652; SEQ ID NO: 6653; SEQ ID NO: 6654; SEQ ID NO: 6655; SEQ ID NO: 6656; SEQ ID NO: 6657; SEQ ID NO: 6658; SEQ ID NO: 6659; SEQ ID NO: 6660; SEQ ID NO: 6661; SEQ ID NO: 6662; SEQ ID NO: 6663; SEQ ID NO: 6664; SEQ ID NO: 6665; SEQ ID NO: 6666; SEQ ID NO: 6667; SEQ ID NO: 6668; SEQ ID NO: 6669; SEQ ID NO: 6670; SEQ ID NO: 6671; SEQ ID NO: 6672; SEQ ID NO: 6673; SEQ ID NO: 6674; SEQ ID NO: 6675; SEQ ID NO: 6676; SEQ ID NO: 6677; SEQ ID NO: 6678; SEQ ID NO: 6679; SEQ ID NO: 6680; SEQ ID NO: 6681; SEQ ID NO: 6682; SEQ ID NO: 6683; SEQ ID NO: 6684; SEQ ID NO: 6685; SEQ ID NO: 6686; SEQ ID NO: 6687; SEQ ID NO: 6688; SEQ ID NO: 6689; SEQ ID NO: 6690; SEQ ID NO: 6691; SEQ ID NO: 6692; SEQ ID NO: 6693; SEQ ID NO: 6694; SEQ ID NO: 6695; SEQ ID NO: 6696; SEQ ID NO: 6697; SEQ ID NO: 6698; SEQ ID NO: 6699; SEQ ID NO: 6700; SEQ ID NO: 6701; SEQ ID NO: 6702; SEQ ID NO: 6703; SEQ ID NO: 6704; SEQ ID NO: 6705; SEQ ID NO: 6706; SEQ ID NO: 6707; SEQ ID NO: 6708; SEQ ID NO: 6709; SEQ ID NO: 6710; SEQ ID NO: 6711; SEQ ID NO: 6712; SEQ ID NO: 6713; SEQ ID NO: 6714; SEQ ID NO: 6715; SEQ ID NO: 6716; SEQ ID NO: 6717; SEQ ID NO: 6718; SEQ ID NO: 6719; SEQ ID NO: 6720; SEQ ID NO: 6721; SEQ ID NO: 6722; SEQ ID NO: 6723; SEQ ID NO: 6724; SEQ ID NO: 6725; SEQ ID NO: 6726; SEQ ID NO: 6727; SEQ ID NO: 6728; SEQ ID NO: 6729; SEQ ID NO: 6730; SEQ ID NO: 6731; SEQ ID NO: 6732; SEQ ID NO: 6733; SEQ ID NO: 6734; SEQ ID NO: 6735; SEQ ID NO: 6736; SEQ ID NO: 6737; SEQ ID NO: 6738; SEQ ID NO: 6739; SEQ ID NO: 6740; SEQ ID NO: 6741; SEQ ID NO: 6742; SEQ ID NO: 6743; SEQ ID NO: 6744; SEQ ID NO: 6745; SEQ ID NO: 6746; SEQ ID NO: 6747; SEQ ID NO: 6748; SEQ ID NO: 6749; SEQ ID NO: 6750; SEQ ID NO: 6751; SEQ ID NO: 6752; SEQ ID NO: 6753; SEQ ID NO: 6754; SEQ ID NO: 6755; SEQ ID NO: 6756; SEQ ID NO: 6757; SEQ ID NO: 6758; SEQ ID NO: 6759; SEQ ID NO: 6760; SEQ ID NO: 6761; SEQ ID NO: 6762; SEQ ID NO: 6763; SEQ ID NO: 6764; SEQ ID NO: 6765; SEQ ID NO: 6766; SEQ ID NO: 6767; SEQ ID NO: 6768; SEQ ID NO: 6769; SEQ ID NO: 6770; SEQ ID NO: 6771; SEQ ID NO: 6772; SEQ ID NO: 6773; SEQ ID NO: 6774; SEQ ID NO: 6775; SEQ ID NO: 6776; SEQ ID NO: 6777; SEQ ID NO: 6778; SEQ ID NO: 6779; SEQ ID NO: 6780; SEQ ID NO: 6781; SEQ ID NO: 6782; SEQ ID NO: 6783; SEQ ID NO: 6784; SEQ ID NO: 6785; SEQ ID NO: 6786; SEQ ID NO: 6787; SEQ ID NO: 6788; SEQ ID NO: 6789; SEQ ID NO: 6790; SEQ ID NO: 6791; SEQ ID NO: 6792; SEQ ID NO: 6793; SEQ ID NO: 6794; SEQ ID NO: 6795; SEQ ID NO: 6796; SEQ ID NO: 6797; SEQ ID NO: 6798; SEQ ID NO: 6799; SEQ ID NO: 6800; SEQ ID NO: 6801; SEQ ID NO: 6802; SEQ ID NO: 6803; SEQ ID NO: 6804; SEQ ID NO: 6805; SEQ ID NO: 6806; SEQ ID NO: 6807; SEQ ID NO: 6808; SEQ ID NO: 6809; SEQ ID NO: 6810; SEQ ID NO: 6811; SEQ ID NO: 6812; SEQ ID NO: 6813; SEQ ID NO: 6814; SEQ ID NO: 6815; SEQ ID NO: 6816; SEQ ID NO: 6817; SEQ ID NO: 6818; SEQ ID NO: 6819; SEQ ID NO: 6820; SEQ ID NO: 6821; SEQ ID NO: 6822; SEQ ID NO: 6823; SEQ ID NO: 6824; SEQ ID NO: 6825; SEQ ID NO: 6826; SEQ ID NO: 6827; SEQ ID NO: 6828; SEQ ID NO: 6829; SEQ ID NO: 6830; SEQ ID NO: 6831; SEQ ID NO: 6832; SEQ ID NO: 6833; SEQ ID NO: 6834; SEQ ID NO: 6835; SEQ ID NO: 6836; SEQ ID NO: 6837; SEQ ID NO: 6838; SEQ ID NO: 6839; SEQ ID NO: 6840; SEQ ID NO: 6841; SEQ ID NO: 6842; SEQ ID NO: 6843; SEQ ID NO: 6844; SEQ ID NO: 6845; SEQ ID NO: 6846; SEQ ID NO: 6847; SEQ ID NO: 6848; SEQ ID NO: 6849; SEQ ID NO: 6850; SEQ ID NO: 6851; SEQ ID NO: 6852; SEQ ID NO: 6853; SEQ ID NO: 6854; SEQ ID NO: 6855; SEQ ID NO: 6856; SEQ ID NO: 6857; SEQ ID NO: 6858; SEQ ID NO: 6859; SEQ ID NO: 6860; SEQ ID NO: 6861; SEQ ID NO: 6862; SEQ ID NO: 6863; SEQ ID NO: 6864; SEQ ID NO: 6865; SEQ ID NO: 6866; SEQ ID NO: 6867; SEQ ID NO: 6868; SEQ ID NO: 6869; SEQ ID NO: 6870; SEQ ID NO: 6871; SEQ ID NO: 6872; SEQ ID NO: 6873; SEQ ID NO: 6874; SEQ ID NO: 6875; SEQ ID NO: 6876; SEQ ID NO: 6877; SEQ ID NO: 6878; SEQ ID NO: 6879; SEQ ID NO: 6880; SEQ ID NO: 6881; SEQ ID NO: 6882; SEQ ID NO: 6883; SEQ ID NO: 6884; SEQ ID NO: 6885; SEQ ID NO: 6886; SEQ ID NO: 6887; SEQ ID NO: 6888; SEQ ID NO: 6889; SEQ ID NO: 6890; SEQ ID NO: 6891; SEQ ID NO: 6892; SEQ ID NO: 6893; SEQ ID NO: 6894; SEQ ID NO: 6895; SEQ ID NO: 6896; SEQ ID NO: 6897; SEQ ID NO: 6898; SEQ ID NO: 6899; SEQ ID NO: 6900; SEQ ID NO: 6901; SEQ ID NO: 6902; SEQ ID NO: 6903; SEQ ID NO: 6904; SEQ ID NO: 6905; SEQ ID NO: 6906; SEQ ID NO: 6907; SEQ ID NO: 6908; SEQ ID NO: 6909; SEQ ID NO: 6910; SEQ ID NO: 6911; SEQ ID NO: 6912; SEQ ID NO: 6913; SEQ ID NO: 6914; SEQ ID NO: 6915; SEQ ID NO: 6916; SEQ ID NO: 6917; SEQ ID NO: 6918; SEQ ID NO: 6919; SEQ ID NO: 6920; SEQ ID NO: 6921; SEQ ID NO: 6922; SEQ ID NO: 6923; SEQ ID NO: 6924; SEQ ID NO: 6925; SEQ ID NO: 6926; SEQ ID NO: 6927; SEQ ID NO: 6928; SEQ ID NO: 6929; SEQ ID NO: 6930; SEQ ID NO: 6931; SEQ ID NO: 6932; SEQ ID NO: 6933; SEQ ID NO: 6934; SEQ ID NO: 6935; SEQ ID NO: 6936; SEQ ID NO: 6937; SEQ ID NO: 6938; SEQ ID NO: 6939; SEQ ID NO: 6940; SEQ ID NO: 6941; SEQ ID NO: 6942; SEQ ID NO: 6943; SEQ ID NO: 6944; SEQ ID NO: 6945; SEQ ID NO: 6946; SEQ ID NO: 6947; SEQ ID NO: 6948; SEQ ID NO: 6949; SEQ ID NO: 6950; SEQ ID NO: 6951; SEQ ID NO: 6952; SEQ ID NO: 6953; SEQ ID NO: 6954; SEQ ID NO: 6955; SEQ ID NO: 6956; SEQ ID NO: 6957; SEQ ID NO: 6958; SEQ ID NO: 6959; SEQ ID NO: 6960; SEQ ID NO: 6961; SEQ ID NO: 6962; SEQ ID NO: 6963; SEQ ID NO: 6964; SEQ ID NO: 6965; SEQ ID NO: 6966; SEQ ID NO: 6967; SEQ ID NO: 6968; SEQ ID NO: 6969; SEQ ID NO: 6970; SEQ ID NO: 6971; SEQ ID NO: 6972; SEQ ID NO: 6973; SEQ ID NO: 6974; SEQ ID NO: 6975; SEQ ID NO: 6976; SEQ ID NO: 6977; SEQ ID NO: 6978; SEQ ID NO: 6979; SEQ ID NO: 6980; SEQ ID NO: 6981; SEQ ID NO: 6982; SEQ ID NO: 6983; SEQ ID NO: 6984; SEQ ID NO: 6985; SEQ ID NO: 6986; SEQ ID NO: 6987; SEQ ID NO: 6988; SEQ ID NO: 6989; SEQ ID NO: 6990; SEQ ID NO: 6991; SEQ ID NO: 6992; SEQ ID NO: 6993; SEQ ID NO: 6994; SEQ ID NO: 6995; SEQ ID NO: 6996; SEQ ID NO: 6997; SEQ ID NO: 6998; SEQ ID NO: 6999; SEQ ID NO: 7000; SEQ ID NO: 7001; SEQ ID NO: 7002; SEQ ID NO: 7003; SEQ ID NO: 7004; SEQ ID NO: 7005; SEQ ID NO: 7006; SEQ ID NO: 7007; SEQ ID NO: 7008; SEQ ID NO: 7009; SEQ ID NO: 7010; SEQ ID NO: 7011; SEQ ID NO: 7012; SEQ ID NO: 7013; SEQ ID NO: 7014; SEQ ID NO: 7015; SEQ ID NO: 7016; SEQ ID NO: 7017; SEQ ID NO: 7018; SEQ ID NO: 7019; SEQ ID NO: 7020; SEQ ID NO: 7021; SEQ ID NO: 7022; SEQ ID NO: 7023; SEQ ID NO: 7024; SEQ ID NO: 7025; SEQ ID NO: 7026; SEQ ID NO: 7027; SEQ ID NO: 7028; SEQ ID NO: 7029; SEQ ID NO: 7030; SEQ ID NO: 7031; SEQ ID NO: 7032; SEQ ID NO: 7033; SEQ ID NO: 7034; SEQ ID NO: 7035; SEQ ID NO: 7036; SEQ ID NO: 7037; SEQ ID NO: 7038; SEQ ID NO: 7039; SEQ ID NO: 7040; SEQ ID NO: 7041; SEQ ID NO: 7042; SEQ ID NO: 7043; SEQ ID NO: 7044; SEQ ID NO: 7045; SEQ ID NO: 7046; SEQ ID NO: 7047; SEQ ID NO: 7048; SEQ ID NO: 7049; SEQ ID NO: 7050; SEQ ID NO: 7051; SEQ ID NO: 7052; SEQ ID NO: 7053; SEQ ID NO: 7054; SEQ ID NO: 7055; SEQ ID NO-7056; SEQ ID NO: 7057; SEQ ID NO: 7058; SEQ ID NO: 7059; SEQ ID NO: 7060; SEQ ID NO: 7061; SEQ ID NO: 7062; SEQ ID NO: 7063; SEQ ID NO: 7064; SEQ ID NO: 7065; SEQ ID NO: 7066; SEQ ID NO: 7067; SEQ ID NO: 7068; SEQ ID NO: 7069; SEQ ID NO: 7070; SEQ ID NO: 7071; SEQ ID NO: 7072; SEQ ID NO: 7073; SEQ ID NO: 7074; SEQ ID NO: 7075; SEQ ID NO: 7076; SEQ ID NO: 7077; SEQ ID NO: 7078; SEQ ID NO: 7079; SEQ ID NO: 7080; SEQ ID NO: 7081; SEQ ID NO: 7082; SEQ ID NO: 7083; SEQ ID NO: 7084; SEQ ID NO: 7085; SEQ ID NO: 7086; SEQ ID NO: 7087; SEQ ID NO: 7088; SEQ ID NO: 7089; SEQ ID NO: 7090; SEQ ID NO: 7091; SEQ ID NO: 7092; SEQ ID NO: 7093; SEQ ID NO: 7094; SEQ ID NO: 7095; SEQ ID NO: 7096; SEQ ID NO: 7097; SEQ ID NO: 7098; SEQ ID NO: 7099; SEQ ID NO: 7100; SEQ ID NO: 7101; SEQ ID NO: 7102; SEQ ID NO: 7103; SEQ ID NO: 7104; SEQ ID NO: 7105; SEQ ID NO: 7106; SEQ ID NO: 7107; SEQ ID NO: 7108; SEQ ID NO: 7109; SEQ ID NO: 7110; SEQ ID NO: 7111; SEQ ID NO: 7112; SEQ ID NO: 7113; SEQ ID NO: 7114; SEQ ID NO: 7115; SEQ ID NO: 7116; SEQ ID NO: 7117; SEQ ID NO: 7118; SEQ ID NO: 7119; SEQ ID NO: 7120; SEQ ID NO: 7121; SEQ ID NO: 7122; SEQ ID NO: 7123; SEQ ID NO: 7124; SEQ ID NO: 7125; SEQ ID NO: 7126; SEQ ID NO: 7127; SEQ ID NO: 7128; SEQ ID NO: 7129; SEQ ID NO: 7130; SEQ ID NO: 7131; SEQ ID NO: 7132; SEQ ID NO: 7133; SEQ ID NO: 7134; SEQ ID NO: 7135; SEQ ID NO: 7136; SEQ ID NO: 7137; SEQ ID NO: 7138; SEQ ID NO: 7139; SEQ ID NO: 7140; SEQ ID NO: 7141; SEQ ID NO: 7142; SEQ ID NO: 7143; SEQ ID NO: 7144; SEQ ID NO: 7145; SEQ ID NO: 7146; SEQ ID NO: 7147; SEQ ID NO: 7148; SEQ ID NO: 7149; SEQ ID NO: 7150; SEQ ID NO: 7151; SEQ ID NO: 7152; SEQ ID NO: 7153; SEQ ID NO: 7154; SEQ ID NO: 7155; SEQ ID NO: 7156; SEQ ID NO: 7157; SEQ ID NO: 7158; SEQ ID NO: 7159; SEQ ID NO: 7160; SEQ ID NO: 7161; SEQ ID NO: 7162; SEQ ID NO: 7163; SEQ ID NO: 7164; SEQ ID NO: 7165; SEQ ID NO: 7166; SEQ ID NO: 7167; SEQ ID NO: 7168; SEQ ID NO: 7169; SEQ ID NO: 7170; SEQ ID NO: 7171; SEQ ID NO: 7172; SEQ ID NO: 7173; SEQ ID NO: 7174; SEQ ID NO: 7175; SEQ ID NO: 7176; SEQ ID NO: 7177; SEQ ID NO: 7178; SEQ ID NO: 7179; SEQ ID NO: 7180; SEQ ID NO: 7181; SEQ ID NO: 7182; SEQ ID NO: 7183; SEQ ID NO: 7184; SEQ ID NO: 7185; SEQ ID NO: 7186; SEQ ID NO: 7187; SEQ ID NO: 7188; SEQ ID NO: 7189; SEQ ID NO: 7190; SEQ ID NO: 7191; SEQ ID NO: 7192; SEQ ID NO: 7193; SEQ ID NO: 7194; SEQ ID NO: 7195; SEQ ID NO: 7196; SEQ ID NO: 7197; SEQ ID NO: 7198; SEQ ID NO: 7199; SEQ ID NO: 7200; SEQ ID NO: 7201; SEQ ID NO: 7202; SEQ ID NO: 7203; SEQ ID NO: 7204; SEQ ID NO: 7205; SEQ ID NO: 7206; SEQ ID NO: 7207; SEQ ID NO: 7208; SEQ ID NO: 7209; SEQ ID NO: 7210; SEQ ID NO: 7211; SEQ ID NO: 7212; SEQ ID NO: 7213; SEQ ID NO: 7214; SEQ ID NO: 7215; SEQ ID NO: 7216; SEQ ID NO: 7217; SEQ ID NO: 7218; SEQ ID NO: 7219; SEQ ID NO: 7220; SEQ ID NO: 7221; SEQ ID NO: 7222; SEQ ID NO: 7223; SEQ ID NO: 7224; SEQ ID NO: 7225; SEQ ID NO: 7226; SEQ ID NO: 7227; SEQ ID NO: 7228; SEQ ID NO: 7229; SEQ ID NO: 7230; SEQ ID NO: 7231; SEQ ID NO: 7232; SEQ ID NO: 7233; SEQ ID NO: 7234; SEQ ID NO: 7235; SEQ ID NO: 7236; SEQ ID NO: 7237; SEQ ID NO: 7238; SEQ ID NO: 7239; SEQ ID NO: 7240; SEQ ID NO: 7241; SEQ ID NO: 7242; SEQ ID NO: 7243; SEQ ID NO: 7244; SEQ ID NO: 7245; SEQ ID NO: 7246; SEQ ID NO: 7247; SEQ ID NO: 7248; SEQ ID NO: 7249; SEQ ID NO: 7250; SEQ ID NO: 7251; SEQ ID NO: 7252; SEQ ID NO: 7253; SEQ ID NO: 7254; SEQ ID NO: 7255; SEQ ID NO: 7256; SEQ ID NO: 7257; SEQ ID NO: 7258; SEQ ID NO: 7259; SEQ ID NO: 7260; SEQ ID NO: 7261; SEQ ID NO: 7262; SEQ ID NO: 7263; SEQ ID NO: 7264; SEQ ID NO: 7265; SEQ ID NO: 7266; SEQ ID NO: 7267; SEQ ID NO: 7268; SEQ ID NO: 7269; SEQ ID NO: 7270; SEQ ID NO: 7271; SEQ ID NO: 7272; SEQ ID NO: 7273; SEQ ID NO: 7274; SEQ ID NO: 7275; SEQ ID NO: 7276; SEQ ID NO: 7277; SEQ ID NO: 7278; SEQ ID NO: 7279; SEQ ID NO: 7280; SEQ ID NO: 7281; SEQ ID NO: 7282; SEQ ID NO: 7283; SEQ ID NO: 7284; SEQ ID NO: 7285; SEQ ID NO: 7286; SEQ ID NO: 7287; SEQ ID NO: 7288; SEQ ID NO: 7289; SEQ ID NO: 7290; SEQ ID NO: 7291; SEQ ID NO: 7292; SEQ ID NO:7293; SEQ ID NO: 7294; SEQ ID NO: 7295; SEQ ID NO: 7296; SEQ ID NO: 7297; SEQ ID NO: 7298; SEQ ID NO: 7299; SEQ ID NO: 7300; SEQ ID NO: 7301; SEQ ID NO: 7302; SEQ ID NO: 7303; SEQ ID NO: 7304; SEQ ID NO: 7305; SEQ ID NO: 7306; SEQ ID NO: 7307; SEQ ID NO: 7308; SEQ ID NO: 7309; SEQ ID NO: 7310; SEQ ID NO: 7311; SEQ ID NO: 7312; SEQ ID NO: 7313; SEQ ID NO: 7314; SEQ ID NO: 7315; SEQ ID NO: 7316; SEQ ID NO: 7317; SEQ ID NO: 7318; SEQ ID NO: 7319; SEQ ID NO: 7320; SEQ ID NO: 7321; SEQ ID NO: 7322; SEQ ID NO: 7323; SEQ ID NO: 7324; SEQ ID NO: 7325; SEQ ID NO: 7326; SEQ ID NO: 7327; SEQ ID NO: 7328; SEQ ID NO: 7329; SEQ ID NO: 7330; SEQ ID NO: 7331; SEQ ID NO: 7332; SEQ ID NO: 7333; SEQ ID NO: 7334; SEQ ID NO: 7335; SEQ ID NO: 7336; SEQ ID NO: 7337; SEQ ID NO: 7338; SEQ ID NO: 7339; SEQ ID NO: 7340; SEQ ID NO: 7341; SEQ ID NO: 7342; SEQ ID NO: 7343; SEQ ID NO: 7344; SEQ ID NO: 7345; SEQ ID NO: 7346; SEQ ID NO: 7347; SEQ ID NO: 7348; SEQ ID NO: 7349; SEQ ID NO: 7350; SEQ ID NO: 7351; SEQ ID NO: 7352; SEQ ID NO: 7353; SEQ ID NO: 7354; SEQ ID NO: 7355; SEQ ID NO: 7356; SEQ ID NO: 7357; SEQ ID NO: 7358; SEQ ID NO: 7359; SEQ ID NO: 7360; SEQ ID NO: 7361; SEQ ID NO: 7362; SEQ ID NO: 7363; SEQ ID NO: 7364; SEQ ID NO: 7365; SEQ ID NO: 7366; SEQ ID NO: 7367; SEQ ID NO: 7368; SEQ ID NO: 7369; SEQ ID NO: 7370; SEQ ID NO: 7371; SEQ ID NO: 7372; SEQ ID NO: 7373; SEQ ID NO: 7374; SEQ ID NO: 7375; SEQ ID NO: 7376; SEQ ID NO: 7377; SEQ ID NO: 7378; SEQ ID NO: 7379; SEQ ID NO: 7380; SEQ ID NO: 7381; SEQ ID NO: 7382; SEQ ID NO: 7383; SEQ ID NO: 7384; SEQ ID NO: 7385; SEQ ID NO: 7386; SEQ ID NO: 7387; SEQ ID NO: 7388; SEQ ID NO: 7389; SEQ ID NO: 7390; SEQ ID NO: 7391; SEQ ID NO: 7392; SEQ ID NO: 7393; SEQ ID NO: 7394; SEQ ID NO: 7395; SEQ ID NO: 7396; SEQ ID NO: 7397; SEQ ID NO: 7398; SEQ ID NO: 7399; SEQ ID NO: 7400; SEQ ID NO: 7401; SEQ ID NO: 7402; SEQ ID NO: 7403; SEQ ID NO: 7404; SEQ ID NO: 7405; SEQ ID NO: 7406; SEQ ID NO: 7407; SEQ ID NO: 7408; SEQ ID NO: 7409; SEQ ID NO: 7410; SEQ ID NO: 7411; SEQ ID NO: 7412; SEQ ID NO: 7413; SEQ ID NO: 7414; SEQ ID NO: 7415; SEQ ID NO: 7416; SEQ ID NO: 7417; SEQ ID NO: 7418; SEQ ID NO: 7419; SEQ ID NO: 7420; SEQ ID NO: 7421; SEQ ID NO: 7422; SEQ ID NO: 7423; SEQ ID NO: 7424; SEQ ID NO: 7425; SEQ ID NO: 7426; SEQ ID NO: 7427; SEQ ID NO: 7428; SEQ ID NO: 7429; SEQ ID NO: 7430; SEQ ID NO: 7431; SEQ ID NO: 7432; SEQ ID NO: 7433; SEQ ID NO: 7434; SEQ ID NO: 7435; SEQ ID NO: 7436; SEQ ID NO: 7437; SEQ ID NO: 7438; SEQ ID NO: 7439; SEQ ID NO: 7440; SEQ ID NO: 7441; SEQ ID NO: 7442; SEQ ID NO: 7443; SEQ ID NO: 7444; SEQ ID NO: 7445; SEQ ID NO: 7446; SEQ ID NO: 7447; SEQ ID NO: 7448; SEQ ID NO: 7449; SEQ ID NO: 7450; SEQ ID NO: 7451; SEQ ID NO: 7452; SEQ ID NO: 7453; SEQ ID NO: 7454; SEQ ID NO: 7455; SEQ ID NO: 7456; SEQ ID NO: 7457; SEQ ID NO: 7458; SEQ ID NO: 7459; SEQ ID NO: 7460; SEQ ID NO: 7461; SEQ ID NO: 7462; SEQ ID NO: 7463; SEQ ID NO: 7464; SEQ ID NO: 7465; SEQ ID NO: 7466; SEQ ID NO: 7467; SEQ ID NO: 7468; SEQ ID NO: 7469; SEQ ID NO: 7470; SEQ ID NO: 7471; SEQ ID NO: 7472; SEQ ID NO: 7473; SEQ ID NO: 7474; SEQ ID NO: 7475; SEQ ID NO: 7476; SEQ ID NO: 7477; SEQ ID NO: 7478; SEQ ID NO: 7479; SEQ ID NO: 7480; SEQ ID NO: 7481; SEQ ID NO: 7482; SEQ ID NO: 7483; SEQ ID NO: 7484; SEQ ID NO: 7485; SEQ ID NO: 7486; SEQ ID NO: 7487; SEQ ID NO: 7488; SEQ ID NO: 7489; SEQ ID NO: 7490; SEQ ID NO: 7491; SEQ ID NO: 7492; SEQ ID NO: 7493; SEQ ID NO: 7494; SEQ ID NO: 7495; SEQ ID NO: 7496; SEQ ID NO: 7497; SEQ ID NO: 7498; SEQ ID NO: 7499; SEQ ID NO: 7500; SEQ ID NO: 7501; SEQ ID NO: 7502; SEQ ID NO: 7503; SEQ ID NO: 7504; SEQ ID NO: 7505; SEQ ID NO: 7506; SEQ ID NO: 7507; SEQ ID NO: 7508; SEQ ID NO: 7509; SEQ ID NO: 7510; SEQ ID NO: 7511; SEQ ID NO: 7512; SEQ ID NO: 7513; SEQ ID NO: 7514; SEQ ID NO: 7515; SEQ ID NO: 7516; SEQ ID NO: 7517; SEQ ID NO: 7518; SEQ ID NO: 7519; SEQ ID NO: 7520; SEQ ID NO: 7521; SEQ ID NO: 7522; SEQ ID NO: 7523; SEQ ID NO: 7524; SEQ ID NO: 7525; SEQ ID NO: 7526; SEQ ID NO: 7527; SEQ ID NO: 7528; SEQ ID NO: 7529; SEQ ID NO: 7530; SEQ ID NO: 7531; SEQ ID NO: 7532; SEQ ID NO: 7533; SEQ ID NO: 7534; SEQ ID NO: 7535; SEQ ID NO: 7536; SEQ ID NO: 7537; SEQ ID NO: 7538; SEQ ID NO: 7539; SEQ ID NO: 7540; SEQ ID NO: 7541; SEQ ID NO: 7542; SEQ ID NO: 7543; SEQ ID NO: 7544; SEQ ID NO: 7545; SEQ ID NO: 7546; SEQ ID NO: 7547; SEQ ID NO: 7548; SEQ ID NO: 7549; SEQ ID NO: 7550; SEQ ID NO: 7551; SEQ ID NO: 7552; SEQ ID NO: 7553; SEQ ID NO: 7554; SEQ ID NO: 7555; SEQ ID NO: 7556; SEQ ID NO: 7557; SEQ ID NO: 7558; SEQ ID NO: 7559; SEQ ID NO: 7560; SEQ ID NO: 7561; SEQ ID NO: 7562; SEQ ID NO: 7563; SEQ ID NO: 7564; SEQ ID NO: 7565; SEQ ID NO: 7566; SEQ ID NO: 7567; SEQ ID NO: 7568; SEQ ID NO: 7569; SEQ ID NO: 7570; SEQ ID NO: 7571; SEQ ID NO: 7572; SEQ ID NO: 7573; SEQ ID NO: 7574; SEQ ID NO: 7575; SEQ ID NO: 7576; SEQ ID NO: 7577; SEQ ID NO: 7578; SEQ ID NO: 7579; SEQ ID NO: 7580; SEQ ID NO: 7581; SEQ ID NO: 7582; SEQ ID NO: 7583; SEQ ID NO: 7584; SEQ ID NO: 7585; SEQ ID NO: 7586; SEQ ID NO: 7587; SEQ ID NO: 7588; SEQ ID NO: 7589; SEQ ID NO: 7590; SEQ ID NO: 7591; SEQ ID NO: 7592; SEQ ID NO: 7593; SEQ ID NO: 7594; SEQ ID NO: 7595; SEQ ID NO: 7596; SEQ ID NO: 7597; SEQ ID NO: 7598; SEQ ID NO: 7599; SEQ ID NO: 7600; SEQ ID NO: 7601; SEQ ID NO: 7602; SEQ ID NO: 7603; SEQ ID NO: 7604; SEQ ID NO: 7605; SEQ ID NO: 7606; SEQ ID NO: 7607; SEQ ID NO: 7608; SEQ ID NO: 7609; SEQ ID NO: 7610; SEQ ID NO: 7611; SEQ ID NO: 7612; SEQ ID NO: 7613; SEQ ID NO: 7614; SEQ ID NO: 7615; SEQ ID NO: 7616; SEQ ID NO: 7617; SEQ ID NO: 7618; SEQ ID NO: 7619; SEQ ID NO: 7620; SEQ ID NO: 7621; SEQ ID NO: 7622; SEQ ID NO: 7623; SEQ ID NO: 7624; SEQ ID NO: 7625; SEQ ID NO: 7626; SEQ ID NO: 7627; SEQ ID NO: 7628; SEQ ID NO: 7629; SEQ ID NO: 7630; SEQ ID NO: 7631; SEQ ID NO: 7632; SEQ ID NO: 7633; SEQ ID NO: 7634; SEQ ID NO: 7635; SEQ ID NO: 7636; SEQ ID NO: 7637; SEQ ID NO: 7638; SEQ ID NO: 7639; SEQ ID NO: 7640; SEQ ID NO: 7641; SEQ ID NO: 7642; SEQ ID NO: 7643; SEQ ID NO: 7644; SEQ ID NO: 7645; SEQ ID NO: 7646; SEQ ID NO: 7647; SEQ ID NO: 7648; SEQ ID NO: 7649; SEQ ID NO: 7650; SEQ ID NO: 7651; SEQ ID NO: 7652; SEQ ID NO: 7653; SEQ ID NO: 7654; SEQ ID NO: 7655; SEQ ID NO: 7656; SEQ ID NO: 7657; SEQ ID NO: 7658; SEQ ID NO: 7659; SEQ ID NO: 7660; SEQ ID NO: 7661; SEQ ID NO: 7662; SEQ ID NO: 7663; SEQ ID NO: 7664; SEQ ID NO: 7665; SEQ ID NO: 7666; SEQ ID NO: 7667; SEQ ID NO: 7668; SEQ ID NO: 7669; SEQ ID NO: 7670; SEQ ID NO: 7671; SEQ ID NO: 7672; SEQ ID NO: 7673; SEQ ID NO: 7674; SEQ ID NO: 7675; SEQ ID NO: 7676; SEQ ID NO: 7677; SEQ ID NO: 7678; SEQ ID NO: 7679; SEQ ID NO: 7680; SEQ ID NO: 7681; SEQ ID NO: 7682; SEQ ID NO: 7683; SEQ ID NO: 7684; SEQ ID NO: 7685; SEQ ID NO: 7686; SEQ ID NO: 7687; SEQ ID NO: 7688; SEQ ID NO: 7689; SEQ ID NO: 7690; SEQ ID NO: 7691; SEQ ID NO: 7692; SEQ ID NO: 7693; SEQ ID NO: 7694; SEQ ID NO: 7695; SEQ ID NO: 7696; SEQ ID NO: 7697; SEQ ID NO: 7698; SEQ ID NO: 7699; SEQ ID NO: 7700; SEQ ID NO: 7701; SEQ ID NO: 7702; SEQ ID NO: 7703; SEQ ID NO: 7704; SEQ ID NO: 7705; SEQ ID NO: 7706; SEQ ID NO: 7707; SEQ ID NO: 7708; SEQ ID NO: 7709; SEQ ID NO: 7710; SEQ ID NO: 7711; SEQ ID NO: 7712; SEQ ID NO: 7713; SEQ ID NO: 7714; SEQ ID NO: 7715; SEQ ID NO: 7716; SEQ ID NO: 7717; SEQ ID NO: 7718; SEQ ID NO: 7719; SEQ ID NO: 7720; SEQ ID NO: 7721; SEQ ID NO: 7722; SEQ ID NO: 7723; SEQ ID NO: 7724; SEQ ID NO: 7725; SEQ ID NO: 7726; SEQ ID NO: 7727; SEQ ID NO: 7728; SEQ ID NO: 7729; SEQ ID NO: 7730; SEQ ID NO: 7731; SEQ ID NO: 7732; SEQ ID NO: 7733; SEQ ID NO: 7734; SEQ ID NO: 7735; SEQ ID NO: 7736; SEQ ID NO: 7737; SEQ ID NO: 7738; SEQ ID NO: 7739; SEQ ID NO: 7740; SEQ ID NO: 7741; SEQ ID NO: 7742; SEQ ID NO: 7743; SEQ ID NO: 7744; SEQ ID NO: 7745; SEQ ID NO: 7746; SEQ ID NO: 7747; SEQ ID NO: 7748; SEQ ID NO: 7749; SEQ ID NO: 7750; SEQ ID NO: 7751; SEQ ID NO: 7752; SEQ ID NO: 7753; SEQ ID NO: 7754; SEQ ID NO: 7755; SEQ ID NO: 7756; SEQ ID NO: 7757; SEQ ID NO: 7758; SEQ ID NO: 7759; SEQ ID NO: 7760; SEQ ID NO: 7761; SEQ ID NO: 7762; SEQ ID NO: 7763; SEQ ID NO: 7764; SEQ ID NO: 7765; SEQ ID NO: 7766; SEQ ID NO: 7767; SEQ ID NO: 7768; SEQ ID NO: 7769; SEQ ID NO: 7770; SEQ ID NO: 7771; SEQ ID NO: 7772; SEQ ID NO: 7773; SEQ ID NO: 7774; SEQ ID NO: 7775; SEQ ID NO: 7776; SEQ ID NO: 7777; SEQ ID NO: 7778; SEQ ID NO: 7779; SEQ ID NO: 7780; SEQ ID NO: 7781; SEQ ID NO: 7782; SEQ ID NO: 7783; SEQ ID NO: 7784; SEQ ID NO: 7785; SEQ ID NO: 7786; SEQ ID NO: 7787; SEQ ID NO: 7788; SEQ ID NO: 7789; SEQ ID NO: 7790; SEQ ID NO: 7791; SEQ ID NO: 7792; SEQ ID NO: 7793; SEQ ID NO: 7794; SEQ ID NO: 7795; SEQ ID NO: 7796; SEQ ID NO: 7797; SEQ ID NO: 7798; SEQ ID NO: 7799; SEQ ID NO: 7800; SEQ ID NO: 7801; SEQ ID NO: 7802; SEQ ID NO: 7803; SEQ ID NO: 7804; SEQ ID NO: 7805; SEQ ID NO: 7806; SEQ ID NO: 7807; SEQ ID NO: 7808; SEQ ID NO: 7809; SEQ ID NO: 7810; SEQ ID NO: 7811; SEQ ID NO: 7812; SEQ ID NO: 7813; SEQ ID NO: 7814; SEQ ID NO: 7815; SEQ ID NO: 7816; SEQ ID NO: 7817; SEQ ID NO: 7818; SEQ ID NO: 7819; SEQ ID NO: 7820; SEQ ID NO: 7821; SEQ ID NO: 7822; SEQ ID NO: 7823; SEQ ID NO: 7824; SEQ ID NO: 7825; SEQ ID NO: 7826; SEQ ID NO: 7827; SEQ ID NO: 7828; SEQ ID NO: 7829; SEQ ID NO: 7830; SEQ ID NO: 7831; SEQ ID NO: 7832; SEQ ID NO: 7833; SEQ ID NO: 7834; SEQ ID NO: 7835; SEQ ID NO: 7836; SEQ ID NO: 7837; SEQ ID NO: 7838; SEQ ID NO: 7839; SEQ ID NO: 7840; SEQ ID NO: 7841; SEQ ID NO: 7842; SEQ ID NO: 7843; SEQ ID NO: 7844; SEQ ID NO: 7845; SEQ ID NO: 7846; SEQ ID NO: 7847; SEQ ID NO: 7848; SEQ ID NO: 7849; SEQ ID NO: 7850; SEQ ID NO: 7851; SEQ ID NO: 7852; SEQ ID NO: 7853; SEQ ID NO: 7854; SEQ ID NO: 7855; SEQ ID NO: 7856; SEQ ID NO: 7857; SEQ ID NO: 7858; SEQ ID NO: 7859; SEQ ID NO: 7860; SEQ ID NO: 7861; SEQ ID NO: 7862; SEQ ID NO: 7863; SEQ ID NO: 7864; SEQ ID NO: 7865; SEQ ID NO: 7866; SEQ ID NO: 7867; SEQ ID NO: 7868; SEQ ID NO: 7869; SEQ ID NO: 7870; SEQ ID NO: 7871; SEQ ID NO: 7872; SEQ ID NO: 7873; SEQ ID NO: 7874; SEQ ID NO: 7875; SEQ ID NO: 7876; SEQ ID NO: 7877; SEQ ID NO: 7878; SEQ ID NO: 7879; SEQ ID NO: 7880; SEQ ID NO: 7881; SEQ ID NO: 7882; SEQ ID NO. 7883; SEQ ID NO: 7884; SEQ ID NO: 7885; SEQ ID NO: 7886; SEQ ID NO: 788; SEQ ID NO: 7888; SEQ ID NO: 7889; SEQ ID NO: 7890; SEQ ID NO: 7891; SEQ ID NO: 7892; SEQ ID NO: 7893; SEQ ID NO: 7894; SEQ ID NO: 7895; SEQ ID NO: 7896; SEQ ID NO: 7897; SEQ ID NO: 7898; SEQ ID NO: 7899; SEQ ID NO: 7900; SEQ ID NO: 7901; SEQ ID NO: 7902; SEQ ID NO: 7903; SEQ ID NO: 7904; SEQ ID NO: 7905; SEQ ID NO: 7906; SEQ ID NO: 7907; SEQ ID NO: 7908; SEQ ID NO: 7909; SEQ ID NO: 7910; SEQ ID NO: 7911; SEQ ID NO: 7912; SEQ ID NO: 7913; SEQ ID NO: 7914; SEQ ID NO: 7915; SEQ ID NO: 7916; SEQ ID NO: 7917; SEQ ID NO: 7918; SEQ ID NO: 7919; SEQ ID NO: 7920; SEQ ID NO: 7921; SEQ ID NO: 7922; SEQ ID NO: 7923; SEQ ID NO: 7924; SEQ ID NO: 7925; SEQ ID NO: 7926; SEQ ID NO: 7927; SEQ ID NO: 7928; SEQ ID NO: 7929; SEQ ID NO: 7930; SEQ ID NO: 7931; SEQ ID NO: 7932; SEQ ID NO: 7933; SEQ ID NO: 7934; SEQ ID NO: 7935; SEQ ID NO: 7936; SEQ ID NO: 7937; SEQ ID NO: 7938; SEQ ID NO: 7939; SEQ ID NO: 7940; SEQ ID NO: 7941; SEQ ID NO: 7942; SEQ ID NO: 7943; SEQ ID NO: 7944; SEQ ID NO: 7945; SEQ ID NO: 7946; SEQ ID NO: 7947; SEQ ID NO: 7948; SEQ ID NO: 7949; SEQ ID NO: 7950; SEQ ID NO: 7951; SEQ ID NO: 7952; SEQ ID NO: 7953; SEQ ID NO: 7954; SEQ ID NO: 7955; SEQ ID NO: 7956; SEQ ID NO: 7957; SEQ ID NO: 7958; SEQ ID NO: 7959; SEQ ID NO: 7960; SEQ ID NO: 7961; SEQ ID NO: 7962; SEQ ID NO: 7963; SEQ ID NO: 7964; SEQ ID NO: 7965; SEQ ID NO: 7966; SEQ ID NO: 7967; SEQ ID NO: 7968; SEQ ID NO: 7969; SEQ ID NO: 7970; SEQ ID NO: 7971; SEQ ID NO: 7972; SEQ ID NO: 7973; SEQ ID NO: 7974; SEQ ID NO: 7975; SEQ ID NO: 7976; SEQ ID NO: 7977; SEQ ID NO: 7978; SEQ ID NO: 7979; SEQ ID NO: 7980; SEQ ID NO: 7981; SEQ ID NO: 7982; SEQ ID NO: 7983; SEQ ID NO: 7984; SEQ ID NO: 7985; SEQ ID NO: 7986; SEQ ID NO: 7987; SEQ ID NO: 7988; SEQ ID NO: 7989; SEQ ID NO: 7990; SEQ ID NO: 7991; SEQ ID NO: 7992; SEQ ID NO: 7993; SEQ ID NO: 7994; SEQ ID NO: 7995; SEQ ID NO: 7996; SEQ ID NO: 7997; SEQ ID NO: 7998; SEQ ID NO: 7999; SEQ ID NO: 8000; SEQ ID NO: 8001; SEQ ID NO: 8002; SEQ ID NO: 8003; SEQ ID NO: 8004; SEQ ID NO: 8005; SEQ ID NO: 8006; SEQ ID NO: 8007; SEQ ID NO: 8008; SEQ ID NO: 8009; SEQ ID NO: 8010; SEQ ID NO: 8011; SEQ ID NO: 8012; SEQ ID NO: 8013; SEQ ID NO: 8014; SEQ ID NO: 8015; SEQ ID NO: 8016; SEQ ID NO: 8017; SEQ ID NO: 8018; SEQ ID NO: 8019; SEQ ID NO: 8020; SEQ ID NO: 8021; SEQ ID NO: 8022; SEQ ID NO: 8023; SEQ ID NO: 8024; SEQ ID NO: 8025; SEQ ID NO: 8026; SEQ ID NO: 8027; SEQ ID NO: 8028; SEQ ID NO: 8029; SEQ ID NO: 8030; SEQ ID NO: 8031; SEQ ID NO: 8032; SEQ ID NO: 8033; SEQ ID NO: 8034; SEQ ID NO: 8035; SEQ ID NO: 8036; SEQ ID NO: 8037; SEQ ID NO: 8038; SEQ ID NO: 8039; SEQ ID NO: 8040; SEQ ID NO: 8041; SEQ ID NO: 8042; SEQ ID NO: 8043; SEQ ID NO: 8044; SEQ ID NO: 8045; SEQ ID NO: 8046; SEQ ID NO: 8047; SEQ ID NO: 8048; SEQ ID NO: 8049; SEQ ID NO: 8050; SEQ ID NO: 8051; SEQ ID NO: 8052; SEQ ID NO: 8053; SEQ ID NO: 8054; SEQ ID NO: 8055; SEQ ID NO: 8056; SEQ ID NO: 8057; SEQ ID NO: 8058; SEQ ID NO: 8059; SEQ ID NO: 8060; SEQ ID NO: 8061; SEQ ID NO: 8062; SEQ ID NO: 8063; SEQ ID NO: 8064; SEQ ID NO: 8065; SEQ ID NO: 8066; SEQ ID NO: 8067; SEQ ID NO: 8068; SEQ ID NO: 8069; SEQ ID NO: 8070; SEQ ID NO: 8071; SEQ ID NO: 8072; SEQ ID NO: 8073; SEQ ID NO: 8074; SEQ ID NO: 8075; SEQ ID NO: 8076; SEQ ID NO: 8077; SEQ ID NO: 8078; SEQ ID NO: 8079; SEQ ID NO: 8080; SEQ ID NO: 8081; SEQ ID NO: 8082; SEQ ID NO: 8083; SEQ ID NO: 8084; SEQ ID NO: 8085; SEQ ID NO: 8086; SEQ ID NO: 8087; SEQ ID NO: 8088; SEQ ID NO: 8089; SEQ ID NO: 8090; SEQ ID NO: 8091; SEQ ID NO: 8092; SEQ ID NO: 8093; SEQ ID NO: 8094; SEQ ID NO: 8095; SEQ ID NO: 8096; SEQ ID NO: 8097; SEQ ID NO: 8098; SEQ ID NO: 8099; SEQ ID NO: 8100; SEQ ID NO: 8101; SEQ ID NO: 8102; SEQ ID NO: 8103; SEQ ID NO: 8104; SEQ ID NO: 8105; SEQ ID NO: 8106; SEQ ID NO: 8107; SEQ ID NO: 8108; SEQ ID NO: 8109; SEQ ID NO: 8110; SEQ ID NO: 8111; SEQ ID NO: 8112; SEQ ID NO: 8113; SEQ ID NO: 8114; SEQ ID NO: 8115; SEQ ID NO: 8116; SEQ ID NO:8117; SEQ ID NO: 8118; SEQ ID NO: 8119; SEQ ID NO: 8120; SEQ ID NO: 8121; SEQ ID NO: 8122; SEQ ID NO: 8123; SEQ ID NO: 8124; SEQ ID NO: 8125; SEQ ID NO: 8126; SEQ ID NO: 8127; SEQ ID NO: 8128; SEQ ID NO: 8129; SEQ ID NO: 8130; SEQ ID NO: 8131; SEQ ID NO: 8132; SEQ ID NO: 8133; SEQ ID NO: 8134; SEQ ID NO: 8135; SEQ ID NO: 8136; SEQ ID NO: 8137; SEQ ID NO: 8138; SEQ ID NO: 8139; SEQ ID NO: 8140; SEQ ID NO: 8141; SEQ ID NO: 8142; SEQ ID NO: 8143; SEQ ID NO: 8144; SEQ ID NO: 8145; SEQ ID NO: 8146; SEQ ID NO: 8147; SEQ ID NO: 8148; SEQ ID NO: 8149; SEQ ID NO: 8150; SEQ ID NO: 8151; SEQ ID NO: 8152; SEQ ID NO: 8153; SEQ ID NO: 8154; SEQ ID NO: 8155; SEQ ID NO: 8156; SEQ ID NO: 8157; SEQ ID NO: 8158; SEQ ID NO: 8159; SEQ ID NO: 8160; SEQ ID NO: 8161; SEQ ID NO: 8162; SEQ ID NO: 8163; SEQ ID NO: 8164; SEQ ID NO: 8165; SEQ ID NO: 8166; SEQ ID NO: 8167; SEQ ID NO: 8168; SEQ ID NO: 8169; SEQ ID NO: 8170; SEQ ID NO: 8171; SEQ ID NO: 8172; SEQ ID NO: 8173; SEQ ID NO: 8174; SEQ ID NO: 8175; SEQ ID NO: 8176; SEQ ID NO: 8177; SEQ ID NO: 8178; SEQ ID NO: 8179; SEQ ID NO: 8180; SEQ ID NO: 8181; SEQ ID NO: 8182; SEQ ID NO: 8183; SEQ ID NO: 8184; SEQ ID NO: 8185; SEQ ID NO: 8186; SEQ ID NO: 8187; SEQ ID NO: 8188; SEQ ID NO: 8189; SEQ ID NO: 8190; SEQ ID NO: 8191; SEQ ID NO: 8192; SEQ ID NO: 8193; SEQ ID NO: 8194; SEQ ID NO: 8195; SEQ ID NO: 8196; SEQ ID NO: 8197; SEQ ID NO: 8198; SEQ ID NO: 8199; SEQ ID NO: 8200; SEQ ID NO: 8201; SEQ ID NO: 8202; SEQ ID NO: 8203; SEQ ID NO: 8204; SEQ ID NO: 8205; SEQ ID NO: 8206; SEQ ID NO: 8207; SEQ ID NO: 8208; SEQ ID NO: 8209; SEQ ID NO: 8210; SEQ ID NO: 8211; SEQ ID NO: 8212; SEQ ID NO: 8213; SEQ ID NO: 8214; SEQ ID NO: 8215; SEQ ID NO: 8216; SEQ ID NO: 8217; SEQ ID NO: 8218; SEQ ID NO: 8219; SEQ ID NO: 8220; SEQ ID NO: 8221; SEQ ID NO: 8222; SEQ ID NO: 8223; SEQ ID NO: 8224; SEQ ID NO: 8225; SEQ ID NO: 8226; SEQ ID NO: 8227; SEQ ID NO: 8228; SEQ ID NO: 8229; SEQ ID NO: 8230; SEQ ID NO: 8231; SEQ ID NO: 8232; SEQ ID NO: 8233; SEQ ID NO: 8234; SEQ ID NO: 8235; SEQ ID NO: 8236; SEQ ID NO: 8237; SEQ ID NO: 8238; SEQ ID NO: 8239; SEQ ID NO: 8240; SEQ ID NO: 8241; SEQ ID NO: 8242; SEQ ID NO: 8243; SEQ ID NO: 8244; SEQ ID NO: 8245; SEQ ID NO: 8246; SEQ ID NO: 8247; SEQ ID NO: 8248; SEQ ID NO: 8249; SEQ ID NO: 8250; SEQ ID NO: 8251; SEQ ID NO: 8252; SEQ ID NO: 8253; SEQ ID NO: 8254; SEQ ID NO: 8255; SEQ ID NO: 8256; SEQ ID NO: 8257; SEQ ID NO: 8258; SEQ ID NO: 8259; SEQ ID NO: 8260; SEQ ID NO: 8261; SEQ ID NO: 8262; SEQ ID NO: 8263; SEQ ID NO: 8264; SEQ ID NO: 8265; SEQ ID NO: 8266; SEQ ID NO: 8267; SEQ ID NO: 8268; SEQ ID NO: 8269; SEQ ID NO: 8270; SEQ ID NO: 8271; SEQ ID NO: 8272; SEQ ID NO: 8273; SEQ ID NO: 8274; SEQ ID NO: 8275; SEQ ID NO: 8276; SEQ ID NO: 8277; SEQ ID NO: 8278; SEQ ID NO: 8279; SEQ ID NO: 8280; SEQ ID NO: 8281; SEQ ID NO: 8282; SEQ ID NO: 8283; SEQ ID NO: 8284; SEQ ID NO: 8285; SEQ ID NO: 8286; SEQ ID NO: 8287; SEQ ID NO: 8288; SEQ ID NO: 8289; SEQ ID NO: 8290; SEQ ID NO: 8291; SEQ ID NO: 8292; SEQ ID NO: 8293; SEQ ID NO: 8294; SEQ ID NO: 8295; SEQ ID NO: 8296; SEQ ID NO: 8297; SEQ ID NO: 8298; SEQ ID NO: 8299; SEQ ID NO: 8300; SEQ ID NO: 8301; SEQ ID NO: 8302; SEQ ID NO: 8303; SEQ ID NO: 8304; SEQ ID NO: 8305; SEQ ID NO: 8306; SEQ ID NO: 8307; SEQ ID NO: 8308; SEQ ID NO: 8309; SEQ ID NO: 8310; SEQ ID NO: 8311; SEQ ID NO: 8312; SEQ ID NO: 8313; SEQ ID NO: 8314; SEQ ID NO: 8315; SEQ ID NO: 8316; SEQ ID NO: 8317; SEQ ID NO: 8318; SEQ ID NO: 8319; SEQ ID NO: 8320; SEQ ID NO: 8321; SEQ ID NO: 8322; SEQ ID NO: 8323; SEQ ID NO: 8324; SEQ ID NO: 8325; SEQ ID NO: 8326; SEQ ID NO: 8327; SEQ ID NO: 8328; SEQ ID NO: 8329; SEQ ID NO: 8330; SEQ ID NO: 8331; SEQ ID NO: 8332; SEQ ID NO: 8333; SEQ ID NO: 8334; SEQ ID NO: 8335; SEQ ID NO: 8336; SEQ ID NO: 8337; SEQ ID NO: 8338; SEQ ID NO: 8339; SEQ ID NO: 8340; SEQ ID NO: 8341; SEQ ID NO: 8342; SEQ ID NO: 8343; SEQ ID NO: 8344; SEQ ID NO: 8345; SEQ ID NO: 8346; SEQ ID NO: 8347; SEQ ID NO: 8348; SEQ ID NO: 8349; SEQ ID NO: 8350; SEQ ID NO: 8351; SEQ ID NO: 8352; SEQ ID NO: 8353; SEQ ID NO: 8354; SEQ ID NO: 8355; SEQ ID NO: 8356; SEQ ID NO: 8357; SEQ ID NO: 8358; SEQ ID NO: 8359; SEQ ID NO: 8360; SEQ ID NO: 8361; SEQ ID NO: 8362; SEQ ID NO: 8363; SEQ ID NO: 8364; SEQ ID NO: 8365; SEQ ID NO: 8366; SEQ ID NO: 8367; SEQ ID NO: 8368; SEQ ID NO: 8369; SEQ ID NO: 8370; SEQ ID NO: 8371; SEQ ID NO: 8372; SEQ ID NO: 8373; SEQ ID NO: 8374; SEQ ID NO: 8375; SEQ ID NO: 8376; SEQ ID NO: 8377; SEQ ID NO: 8378; SEQ ID NO: 8379; SEQ ID NO: 8380; SEQ ID NO: 8381; SEQ ID NO: 8382; SEQ ID NO: 8383; SEQ ID NO: 8384; SEQ ID NO: 8385; SEQ ID NO: 8386; SEQ ID NO: 8387; SEQ ID NO: 8388; SEQ ID NO: 8389; SEQ ID NO: 8390; SEQ ID NO: 8391; SEQ ID NO: 8392; SEQ ID NO: 8393; SEQ ID NO: 8394; SEQ ID NO: 8395; SEQ ID NO: 8396; SEQ ID NO: 8397; SEQ ID NO: 8398; SEQ ID NO: 8399; SEQ ID NO: 8400; SEQ ID NO: 8401; SEQ ID NO: 8402; SEQ ID NO: 8403; SEQ ID NO: 8404; SEQ ID NO: 8405; SEQ ID NO: 8406; SEQ ID NO: 8407; SEQ ID NO: 8408; SEQ ID NO: 8409; SEQ ID NO: 8410; SEQ ID NO: 8411; SEQ ID NO: 8412; SEQ ID NO: 8413; SEQ ID NO: 8414; SEQ ID NO: 8415; SEQ ID NO: 8416; SEQ ID NO: 8417; SEQ ID NO: 8418; SEQ ID NO: 8419; SEQ ID NO: 8420; SEQ ID NO: 8421; SEQ ID NO: 8422; SEQ ID NO: 8423; SEQ ID NO: 8424; SEQ ID NO: 8425; SEQ ID NO: 8426; SEQ ID NO: 8427; SEQ ID NO: 8428; SEQ ID NO: 8429; SEQ ID NO: 8430; SEQ ID NO: 8431; SEQ ID NO: 8432; SEQ ID NO: 8433; SEQ ID NO: 8434; SEQ ID NO: 8435; SEQ ID NO: 8436; SEQ ID NO: 8437; SEQ ID NO: 8438; SEQ ID NO: 8439; SEQ ID NO: 8440; SEQ ID NO: 8441; SEQ ID NO: 8442; SEQ ID NO: 8443; SEQ ID NO: 8444; SEQ ID NO: 8445; SEQ ID NO: 8446; SEQ ID NO: 8447; SEQ ID NO: 8448; SEQ ID NO: 8449; SEQ ID NO: 8450; SEQ ID NO: 8451; SEQ ID NO: 8452; SEQ ID NO: 8453; SEQ ID NO: 8454; SEQ ID NO: 8455; SEQ ID NO: 8456; SEQ ID NO: 8457; SEQ ID NO: 8458; SEQ ID NO: 8459; SEQ ID NO: 8460; SEQ ID NO: 8461; SEQ ID NO: 8462; SEQ ID NO: 8463; SEQ ID NO: 8464; SEQ ID NO: 8465; SEQ ID NO: 8466; SEQ ID NO: 8467; SEQ ID NO: 8468; SEQ ID NO: 8469; SEQ ID NO: 8470; SEQ ID NO: 8471; SEQ ID NO: 8472; SEQ ID NO: 8473; SEQ ID NO: 8474; SEQ ID NO: 8475; SEQ ID NO: 8476; SEQ ID NO: 8477; SEQ ID NO: 8478; SEQ ID NO: 8479; SEQ ID NO: 8480; SEQ ID NO: 8481; SEQ ID NO: 8482; SEQ ID NO: 8483; SEQ ID NO: 8484; SEQ ID NO: 8485; SEQ ID NO: 8486; SEQ ID NO: 8487; SEQ ID NO: 8488; SEQ ID NO: 8489; SEQ ID NO: 8490; SEQ ID NO: 8491; SEQ ID NO: 8492; SEQ ID NO: 8493; SEQ ID NO: 8494; SEQ ID NO: 8495; SEQ ID NO: 8496; SEQ ID NO: 8497; SEQ ID NO: 8498; SEQ ID NO: 8499; SEQ ID NO: 8500; SEQ ID NO: 8501; SEQ ID NO: 8502; SEQ ID NO: 8503; SEQ ID NO: 8504; SEQ ID NO: 8505; SEQ ID NO: 8506; SEQ ID NO: 8507; SEQ ID NO: 8508; SEQ ID NO: 8509; SEQ ID NO: 8510; SEQ ID NO: 8511; SEQ ID NO: 8512; SEQ ID NO: 8513; SEQ ID NO: 8514; SEQ ID NO: 8515; SEQ ID NO: 8516; SEQ ID NO: 8517; SEQ ID NO: 8518; SEQ ID NO: 8519; SEQ ID NO: 8520; SEQ ID NO: 8521; SEQ ID NO: 8522; SEQ ID NO: 8523; SEQ ID NO: 8524; SEQ ID NO: 8525; SEQ ID NO: 8526; SEQ ID NO: 8527; SEQ ID NO: 8528; SEQ ID NO: 8529; SEQ ID NO: 8530; SEQ ID NO: 8531; SEQ ID NO: 8532; SEQ ID NO: 8533; SEQ ID NO: 8534; SEQ ID NO: 8535; SEQ ID NO: 8536; SEQ ID NO: 8537; SEQ ID NO: 8538; SEQ ID NO: 8539; SEQ ID NO: 8540; SEQ ID NO: 8541; SEQ ID NO: 8542; SEQ ID NO: 8543; SEQ ID NO: 8544; SEQ ID NO: 8545; SEQ ID NO: 8546; SEQ ID NO: 8547; SEQ ID NO: 8548; SEQ ID NO: 8549; SEQ ID NO: 8550; SEQ ID NO: 8551; SEQ ID NO: 8552; SEQ ID NO: 8553; SEQ ID NO: 8554; SEQ ID NO: 8555; SEQ ID NO: 8556; SEQ ID NO: 8557; SEQ ID NO: 8558; SEQ ID NO: 8559; SEQ ID NO: 8560; SEQ ID NO: 8561; SEQ ID NO: 8562; SEQ ID NO: 8563; SEQ ID NO: 8564; SEQ ID NO: 8565; SEQ ID NO: 8566; SEQ ID NO: 8567; SEQ ID NO: 8568; SEQ ID NO: 8569; SEQ ID NO: 8570; SEQ ID NO: 8571; SEQ ID NO: 8572; SEQ ID NO: 8573; SEQ ID NO: 8574; SEQ ID NO: 8575; SEQ ID NO: 8576; SEQ ID NO: 8577; SEQ ID NO: 8578; SEQ ID NO: 8579; SEQ ID NO: 8580; SEQ ID NO: 8581; SEQ ID NO: 8582; SEQ ID NO: 8583; SEQ ID NO: 8584; SEQ ID NO: 8585; SEQ ID NO: 8586; SEQ ID NO: 8587; SEQ ID NO: 8588; SEQ ID NO: 8589; SEQ ID NO: 8590; SEQ ID NO: 8591; SEQ ID NO: 8592; SEQ ID NO: 8593; SEQ ID NO: 8594; SEQ ID NO: 8595; SEQ ID NO: 8596; SEQ ID NO: 8597; SEQ ID NO: 8598; SEQ ID NO: 8599; SEQ ID NO: 8600; SEQ ID NO: 8601; SEQ ID NO: 8602; SEQ ID NO: 8603; SEQ ID NO: 8604; SEQ ID NO: 8605; SEQ ID NO: 8606; SEQ ID NO: 8607; SEQ ID NO: 8608; SEQ ID NO: 8609; SEQ ID NO: 8610; SEQ ID NO: 8611; SEQ ID NO: 8612; SEQ ID NO: 8613; SEQ ID NO: 8614; SEQ ID NO: 8615; SEQ ID NO: 8616; SEQ ID NO: 8617; SEQ ID NO: 8618; SEQ ID NO: 8619; SEQ ID NO: 8620; SEQ ID NO: 8621; SEQ ID NO: 8622; SEQ ID NO: 8623; SEQ ID NO: 8624; SEQ ID NO: 8625; SEQ ID NO: 8626; SEQ ID NO: 8627; SEQ ID NO: 8628; SEQ ID NO: 8629; SEQ ID NO: 8630; SEQ ID NO: 8631; SEQ ID NO: 8632; SEQ ID NO: 8633; SEQ ID NO: 8634; SEQ ID NO: 8635; SEQ ID NO: 8636; SEQ ID NO: 8637; SEQ ID NO: 8638; SEQ ID NO: 8639; SEQ ID NO: 8640; SEQ ID NO: 8641; SEQ ID NO: 8642; SEQ ID NO: 8643; SEQ ID NO: 8644; SEQ ID NO: 8645; SEQ ID NO: 8646; SEQ ID NO: 8647; SEQ ID NO: 8648; SEQ ID NO: 8649; SEQ ID NO: 8650; SEQ ID NO: 8651; SEQ ID NO: 8652; SEQ ID NO: 8653; SEQ ID NO: 8654; SEQ ID NO: 8655; SEQ ID NO: 8656; SEQ ID NO: 8657; SEQ ID NO: 8658; SEQ ID NO: 8659; SEQ ID NO: 8660; SEQ ID NO: 8661; SEQ ID NO: 8662; SEQ ID NO: 8663; SEQ ID NO: 8664; SEQ ID NO: 8665; SEQ ID NO: 8666; SEQ ID NO: 8667; SEQ ID NO: 8668; SEQ ID NO: 8669; SEQ ID NO: 8670; SEQ ID NO: 8671; SEQ ID NO: 8672; SEQ ID NO: 8673; SEQ ID NO: 8674; SEQ ID NO: 8675; SEQ ID NO: 8676; SEQ ID NO: 8677; SEQ ID NO: 8678; SEQ ID NO: 8679; SEQ ID NO: 8680; SEQ ID NO: 8681; SEQ ID NO: 8682; SEQ ID NO: 8683; SEQ ID NO: 8684; SEQ ID NO: 8685; SEQ ID NO: 8686; SEQ ID NO: 8687; SEQ ID NO: 8688; SEQ ID NO: 8689; SEQ ID NO: 8690; SEQ ID NO: 8691; SEQ ID NO: 8692; SEQ ID NO: 8693; SEQ ID NO: 8694; SEQ ID NO: 8695; SEQ ID NO: 8696; SEQ ID NO: 8697; SEQ ID NO: 8698; SEQ ID NO: 8699; SEQ ID NO: 8700; SEQ ID NO: 8701; SEQ ID NO: 8702; SEQ ID NO: 8703; SEQ ID NO: 8704; SEQ ID NO: 8705; SEQ ID NO: 8706; SEQ ID NO: 8707; SEQ ID NO: 8708; SEQ ID NO: 8709; SEQ ID NO: 8710; SEQ ID NO: 8711; SEQ ID NO: 8712; SEQ ID NO: 8713; SEQ ID NO: 8714; SEQ ID NO: 8715; SEQ ID NO: 8716; SEQ ID NO: 8717; SEQ ID NO: 8718; SEQ ID NO: 8719; SEQ ID NO: 8720; SEQ ID NO: 8721; SEQ ID NO: 8722; SEQ ID NO: 8723; SEQ ID NO: 8724; SEQ ID NO: 8725; SEQ ID NO: 8726; SEQ ID NO: 8727; SEQ ID NO: 8728; SEQ ID NO: 8729; SEQ ID NO: 8730; SEQ ID NO: 8731; SEQ ID NO: 8732; SEQ ID NO: 8733; SEQ ID NO: 8734; SEQ ID NO: 8735; SEQ ID NO: 8736; SEQ ID NO: 8737; SEQ ID NO: 8738; SEQ ID NO: 8739; SEQ ID NO: 8740; SEQ ID NO: 8741; SEQ ID NO: 8742; SEQ ID NO: 8743; SEQ ID NO: 8744; SEQ ID NO: 8745; SEQ ID NO: 8746; SEQ ID NO: 8747; SEQ ID NO: 8748; SEQ ID NO: 8749; SEQ ID NO: 8750; SEQ ID NO: 8751; SEQ ID NO: 8752; SEQ ID NO: 8753; SEQ ID NO: 8754; SEQ ID NO: 8755; SEQ ID NO: 8756; SEQ ID NO: 8757; SEQ ID NO: 8758; SEQ ID NO: 8759; SEQ ID NO: 8760; SEQ ID NO: 8761; SEQ ID NO: 8762; SEQ ID NO: 8763; SEQ ID NO: 8764; SEQ ID NO: 8765; SEQ ID NO: 8766; SEQ ID NO: 8767; SEQ ID NO: 8768; SEQ ID NO: 8769; SEQ ID NO: 8770; SEQ ID NO: 8771; SEQ ID NO: 8772; SEQ ID NO: 8773; SEQ ID NO: 8774; SEQ ID NO: 8775; SEQ ID NO: 8776; SEQ ID NO: 8777; SEQ ID NO: 8778; SEQ ID NO: 8779; SEQ ID NO: 8780; SEQ ID NO: 8781; SEQ ID NO: 8782; SEQ ID NO: 8783; SEQ ID NO: 8784; SEQ ID NO: 8785; SEQ ID NO: 8786; SEQ ID NO: 8787; SEQ ID NO: 8788; SEQ ID NO: 8789; SEQ ID NO: 8790; SEQ ID NO: 8791; SEQ ID NO: 8792; SEQ ID NO: 8793; SEQ ID NO: 8794; SEQ ID NO: 8795; SEQ ID NO: 8796; SEQ ID NO: 8797; SEQ ID NO: 8798; SEQ ID NO: 8799; SEQ ID NO: 8800; SEQ ID NO: 8801; SEQ ID NO: 8802; SEQ ID NO: 8803; SEQ ID NO: 8804; SEQ ID NO: 8805; SEQ ID NO: 8806; SEQ ID NO: 8807; SEQ ID NO: 8808; SEQ ID NO: 8809; SEQ ID NO: 8810; SEQ ID NO: 8811; SEQ ID NO: 8812; SEQ ID NO: 8813; SEQ ID NO: 8814; SEQ ID NO: 8815; SEQ ID NO: 8816; SEQ ID NO: 8817; SEQ ID NO: 8818; SEQ ID NO: 8819; SEQ ID NO: 8820; SEQ ID NO: 8821; SEQ ID NO: 8822; SEQ ID NO: 8823; SEQ ID NO: 8824; SEQ ID NO: 8825; SEQ ID NO: 8826; SEQ ID NO: 8827; SEQ ID NO: 8828; SEQ ID NO: 8829; SEQ ID NO: 8830; SEQ ID NO: 8831; SEQ ID NO: 8832; SEQ ID NO: 8833; SEQ ID NO: 8834; SEQ ID NO: 8835; SEQ ID NO: 8836; SEQ ID NO: 8837; SEQ ID NO: 8838; SEQ ID NO: 8839; SEQ ID NO: 8840; SEQ ID NO: 8841; SEQ ID NO: 8842; SEQ ID NO: 8843; SEQ ID NO: 8844; SEQ ID NO: 8845; SEQ ID NO: 8846; SEQ ID NO: 8847; SEQ ID NO: 8848; SEQ ID NO: 8849; SEQ ID NO: 8850; SEQ ID NO: 8851; SEQ ID NO: 8852; SEQ ID NO: 8853; SEQ ID NO: 8854; SEQ ID NO: 8855; SEQ ID NO: 8856; SEQ ID NO: 8857; SEQ ID NO: 8858; SEQ ID NO: 8859; SEQ ID NO: 8860; SEQ ID NO: 8861; SEQ ID NO: 8862; SEQ ID NO: 8863; SEQ ID NO: 8864; SEQ ID NO: 8865; SEQ ID NO: 8866; SEQ ID NO: 8867; SEQ ID NO: 8868; SEQ ID NO: 8869; SEQ ID NO: 8870; SEQ ID NO: 8871; SEQ ID NO: 8872; SEQ ID NO: 8873; SEQ ID NO: 8874; SEQ ID NO: 8875; SEQ ID NO: 8876; SEQ ID NO: 8877; SEQ ID NO: 9705; SEQ ID NO: 9706; SEQ ID NO: 9707; SEQ ID NO: 9708; SEQ ID NO: 9709; SEQ ID NO: 9710; SEQ ID NO: 9711; SEQ ID NO: 9712; SEQ ID NO: 9713; SEQ ID NO: 9714; SEQ ID NO: 9715; SEQ ID NO: 9716; SEQ ID NO: 9717; SEQ ID NO: 9718; SEQ ID NO: 9719; SEQ ID NO: 9720; SEQ ID NO: 9721; SEQ ID NO: 9722; SEQ ID NO: 9723; SEQ ID NO: 9724; SEQ ID NO: 9725; SEQ ID NO: 9726; SEQ ID NO: 9727; SEQ ID NO: 9728; SEQ ID NO: 9729; SEQ ID NO: 9730; SEQ ID NO: 9731; SEQ ID NO: 9732 and SEQ ID NO: 9733.

In one embodiment, the H. pylori cytoplasmic polypeptide or a fragment thereof is an H. pylori polypeptide involved in energy metabolism or a fragment thereof having an amino acid sequence selected from the group consisting of SEQ ID NO: 6338; SEQ ID NO: 6339; SEQ ID NO: 6340; SEQ ID NO: 6341; SEQ ID NO: 6342; SEQ ID NO: 6343; SEQ ID NO: 6344; SEQ ID NO: 6345; SEQ ID NO: 6346; SEQ ID NO: 6347; SEQ ID NO: 6348; SEQ ID NO: 6349; SEQ ID NO: 6350; SEQ ID NO: 6351; SEQ ID NO: 6352; SEQ ID NO: 6353; SEQ ID NO: 6354; SEQ ID NO: 6355; SEQ ID NO: 6356; SEQ ID NO: 6357; SEQ ID NO: 6358; SEQ ID NO: 6359; SEQ ID NO: 6360; SEQ ID NO: 6361; SEQ ID NO: 6362; SEQ ID NO: 6363; SEQ ID NO: 6364; SEQ ID NO: 6365; SEQ ID NO: 6366; SEQ ID NO: 6367; SEQ ID NO: 6368; SEQ ID NO: 6369; SEQ ID NO: 6370; SEQ ID NO: 6371; SEQ ID NO: 6372; SEQ ID NO: 6373; SEQ ID NO: 6374; SEQ ID NO: 6375; SEQ ID NO: 6376; SEQ ID NO: 6377; SEQ ID NO: 6378; SEQ ID NO: 6379; SEQ ID NO: 6380; SEQ ID NO: 6381; SEQ ID NO: 6382; SEQ ID NO: 6383; SEQ ID NO: 6384; SEQ ID NO: 6385; SEQ ID NO: 6386; SEQ ID NO: 6387; SEQ ID NO: 6389; SEQ ID NO: 6390; SEQ ID NO: 6391; SEQ ID NO: 6392; SEQ ID NO: 6393; SEQ ID NO: 6394; SEQ ID NO: 6395; SEQ ID NO: 6396; SEQ ID NO: 6397; SEQ ID NO: 6398; SEQ ID NO: 6399; SEQ ID NO: 6400; SEQ ID NO: 6401; SEQ ID NO: 6402; SEQ ID NO: 6403; SEQ ID NO: 6404; SEQ ID NO: 6405; SEQ ID NO: 6406; SEQ ID NO: 6407; SEQ ID NO: 6408; SEQ ID NO: 6409; SEQ ID NO: 6410; SEQ ID NO: 6411; SEQ ID NO: 6412; SEQ ID NO: 6413; SEQ ID NO: 6414; SEQ ID NO: 6415; SEQ ID NO: 6416; SEQ ID NO: 6417; SEQ ID NO: 6418; SEQ ID NO: 6419; SEQ ID NO: 6420; SEQ ID NO: 6421; SEQ ID NO: 6422; SEQ ID NO: 6423; SEQ ID NO: 6424; SEQ ID NO: 6425; SEQ ID NO: 6426; SEQ ID NO: 6427; SEQ ID NO: 6428; SEQ ID NO: 6429; SEQ ID NO: 6430; SEQ ID NO: 6431; SEQ ID NO: 6432; SEQ ID NO: 6433; SEQ ID NO: 6434; SEQ ID NO: 6435; SEQ ID NO: 6436; SEQ ID NO: 6437; SEQ ID NO: 6438; SEQ ID NO: 6439; SEQ ID NO: 6440; SEQ ID NO: 6441; SEQ ID NO: 6442; SEQ ID NO: 6443; SEQ ID NO: 6444; SEQ ID NO: 6445; SEQ ID NO: 6446; SEQ ID NO: 6447; SEQ ID NO: 6448; SEQ ID NO: 6449; SEQ ID NO: 6450; SEQ ID NO: 6451; SEQ ID NO: 6452; SEQ ID NO: 6453; SEQ ID NO: 6454; SEQ ID NO: 6455; SEQ ID NO: 6456; SEQ ID NO: 6457; SEQ ID NO: 6458; SEQ ID NO: 6459; SEQ ID NO: 6460; SEQ ID NO: 6461; SEQ ID NO: 6462; SEQ ID NO: 6463; SEQ ID NO: 6464; SEQ ID NO: 6465; SEQ ID NO: 6466; SEQ ID NO: 6467; SEQ ID NO: 6468; SEQ ID NO: 6469; SEQ ID NO: 6470; SEQ ID NO: 6471; SEQ ID NO: 6472; SEQ ID NO: 6473; SEQ ID NO: 6474; SEQ ID NO: 6475; SEQ ID NO: 6476; SEQ ID NO: 6477; SEQ ID NO: 6478; SEQ ID NO: 6479; SEQ ID NO: 6480; SEQ ID NO: 6481; SEQ ID NO: 6482; SEQ ID NO: 6483; SEQ ID NO: 6484; SEQ ID NO: 6485; SEQ ID NO: 6486; SEQ ID NO: 6487; SEQ ID NO: 9705 and SEQ ID NO: 9706.

In another embodiment, the H. pylori cytoplasmic polypeptide or a fragment thereof is an H. pylori polypeptide involved in amino acid metabolism or a fragment thereof having an amino acid sequence selected from the group consisting of SEQ ID NO: 6488; SEQ ID NO: 6489; SEQ ID NO: 6490; SEQ ID NO: 6491; SEQ ID NO: 6492; SEQ ID NO: 6493; SEQ ID NO: 6494; SEQ ID NO: 6495; SEQ ID NO: 6496; SEQ ID NO: 6497; SEQ ID NO: 6498; SEQ ID NO: 6499; SEQ ID NO: 6500; SEQ ID NO: 6501; SEQ ID NO: 6502; SEQ ID NO: 6503; SEQ ID NO: 6504; SEQ ID NO: 6505; SEQ ID NO: 6506; SEQ ID NO: 6507; SEQ ID NO: 6508; SEQ ID NO: 6509; SEQ ID NO: 6510; SEQ ID NO: 6511; SEQ ID NO: 6512; SEQ ID NO: 6513; SEQ ID NO: 6514; SEQ ID NO: 6515; SEQ ID NO: 6516; SEQ ID NO: 6517; SEQ ID NO: 6518; SEQ ID NO: 6519; SEQ ID NO: 6520; SEQ ID NO: 6521; SEQ ID NO: 6522; SEQ ID NO: 6523; SEQ ID NO: 6524; SEQ ID NO: 6525; SEQ ID NO: 6526, SEQ ID NO: 6527; SEQ ID NO: 6528; SEQ ID NO: 6529; SEQ ID NO: 6530; SEQ ID NO: 6531; SEQ ID NO: 6532; SEQ ID NO: 6533; SEQ ID NO: 6534; SEQ ID NO: 6535; SEQ ID NO: 6536; SEQ ID NO: 6537; SEQ ID NO: 6538; SEQ ID NO: 6539; SEQ ID NO: 6540; SEQ ID NO: 6541; SEQ ID NO: 6542; SEQ ID NO: 6543; SEQ ID NO: 6544; SEQ ID NO: 6545; SEQ ID NO: 6546; SEQ ID NO: 6547; SEQ ID NO: 6548; SEQ ID NO: 6549; SEQ ID NO: 6550; SEQ ID NO: 6551; SEQ ID NO: 6552; SEQ ID NO: 6553; SEQ ID NO: 6554; SEQ ID NO: 6555; SEQ ID NO: 6556; SEQ ID NO: 6557; SEQ ID NO: 6558; SEQ ID NO: 6559; SEQ ID NO: 6560; SEQ ID NO: 6561; SEQ ID NO: 6562; SEQ ID NO: 6563; SEQ ID NO: 6564; SEQ ID NO: 6565; SEQ ID NO: 6566; SEQ ID NO: 6567; SEQ ID NO: 6568; SEQ ID NO: 6569; SEQ ID NO: 6570; SEQ ID NO: 6571; SEQ ID NO: 6572; SEQ ID NO: 6573; SEQ ID NO: 6574; SEQ ID NO: 6575; SEQ ID NO: 6576; SEQ ID NO: 6577; SEQ ID NO: 6578; SEQ ID NO: 6579; SEQ ID NO: 6580; SEQ ID NO: 6581; SEQ ID NO: 6582; SEQ ID NO: 6583; SEQ ID NO: 6584; SEQ ID NO: 6585; SEQ ID NO: 6586; SEQ ID NO: 6587; SEQ ID NO: 6588; SEQ ID NO: 6589; SEQ ID NO: 6590; SEQ ID NO: 6591; SEQ ID NO: 6592; SEQ ID NO: 6593; SEQ ID NO: 6594; SEQ ID NO: 6595; SEQ ID NO: 6596; SEQ ID NO: 6597; SEQ ID NO: 6598; SEQ ID NO: 6599; SEQ ID NO: 6600; SEQ ID NO: 6601; SEQ ID NO: 6602; SEQ ID NO: 6603; SEQ ID NO: 6604; SEQ ID NO: 6605; SEQ ID NO: 6606; SEQ ID NO: 6607; SEQ ID NO: 6608; SEQ ID NO: 6609; SEQ ID NO: 6610; SEQ ID NO: 6611; SEQ ID NO: 6612; SEQ ID NO: 6613; SEQ ID NO: 6614; SEQ ID NO: 6615; SEQ ID NO: 6616; SEQ ID NO: 6617; SEQ ID NO: 6618; SEQ ID NO: 6619; SEQ ID NO: 6620; SEQ ID NO: 6621; SEQ ID NO: 6622; SEQ ID NO: 6623; SEQ ID NO: 6624; SEQ ID NO: 6625; SEQ ID NO: 6626; SEQ ID NO: 6627; SEQ ID NO: 6628; SEQ ID NO: 6629; SEQ ID NO: 6630; SEQ ID NO: 9707 and SEQ ID NO: 9708.

In another embodiment, the H. pylori cytoplasmic polypeptide or a fragment thereof is an H. pylori polypeptide involved in cofactor metabolism or a fragment thereof having an amino acid sequence selected from the group consisting of SEQ ID NO: 6631; SEQ ID NO: 6632; SEQ ID NO: 6633; SEQ ID NO: 6634; SEQ ID NO: 6635; SEQ ID NO: 6636; SEQ ID NO: 6637; SEQ ID NO: 6638; SEQ ID NO: 6639; SEQ ID NO: 6640; SEQ ID NO: 6641; SEQ ID NO: 6642; SEQ ID NO: 6643; SEQ ID NO: 6644; SEQ ID NO: 6645; SEQ ID NO: 6646; SEQ ID NO: 6647; SEQ ID NO: 6648; SEQ ID NO: 6649; SEQ ID NO: 6650; SEQ ID NO: 6651; SEQ ID NO: 6652; SEQ ID NO: 6653; SEQ ID NO: 6654; SEQ ID NO: 6655; SEQ ID NO: 6656; SEQ ID NO: 6657; SEQ ID NO: 6658; SEQ ID NO: 6659; SEQ ID NO: 6660; SEQ ID NO: 6661; SEQ ID NO: 6662; SEQ ID NO: 6663; SEQ ID NO: 6664; SEQ ID NO: 6665; SEQ ID NO: 6666; SEQ ID NO: 6667; SEQ ID NO: 6668; SEQ ID NO: 6669; SEQ ID NO: 6670; SEQ ID NO: 6671; SEQ ID NO: 6672; SEQ ID NO: 6673; SEQ ID NO: 6674; SEQ ID NO: 6675; SEQ ID NO: 6676; SEQ ID NO: 6677; SEQ ID NO: 6678; SEQ ID NO: 6679; SEQ ID NO: 6680; SEQ ID NO: 6681; SEQ ID NO: 6682; SEQ ID NO: 6683; SEQ ID NO: 6684; SEQ ID NO: 6685; SEQ ID NO: 6686; SEQ ID NO: 6687; SEQ ID NO: 6688; SEQ ID NO: 6689; SEQ ID NO: 6690; SEQ ID NO: 6691; SEQ ID NO: 6692; SEQ ID NO: 6693; SEQ ID NO: 6694; SEQ ID NO: 6695; SEQ ID NO: 6696; SEQ ID NO: 6697; SEQ ID NO: 6698; SEQ ID NO: 6699; SEQ ID NO: 6700; SEQ ID NO: 6701; SEQ ID NO: 6702; SEQ ID NO: 6703; SEQ ID NO: 6704; SEQ ID NO: 6705; SEQ ID NO: 6706; SEQ ID NO: 6707; SEQ ID NO: 6708; SEQ ID NO: 6709; SEQ ID NO: 6710; SEQ ID NO: 6711; SEQ ID NO: 6712; SEQ ID NO: 6713; SEQ ID NO: 6714; SEQ ID NO: 6715; SEQ ID NO: 6716; SEQ ID NO: 6717; SEQ ID NO: 6718; SEQ ID NO: 6719; SEQ ID NO: 6720; SEQ ID NO: 6721; SEQ ID NO: 6722; SEQ ID NO: 6723; SEQ ID NO: 6724; SEQ ID NO: 6725; SEQ ID NO: 6726; SEQ ID NO: 6727; SEQ ID NO: 6728; SEQ ID NO: 6729; SEQ ID NO: 6730; SEQ ID NO: 6731; SEQ ID NO: 6732; SEQ ID NO: 6733; SEQ ID NO: 6734; SEQ ID NO: 6735; SEQ ID NO: 6736; SEQ ID NO: 6737; SEQ ID NO: 6738; SEQ ID NO: 6739; SEQ ID NO: 6740; SEQ ID NO: 6741; SEQ ID NO: 6742; SEQ ID NO: 6743; SEQ ID NO: 6744; SEQ ID NO: 6745; SEQ ID NO: 6746; SEQ ID NO: 6747; SEQ ID NO: 6748; SEQ ID NO: 6749, SEQ ID NO: 6750; SEQ ID NO: 6751; SEQ ID NO: 6752; SEQ ID NO: 6753; SEQ ID NO: 6754; SEQ ID NO: 6755; SEQ ID NO: 6756; SEQ ID NO: 6757; SEQ ID NO: 6758; SEQ ID NO: 6759; SEQ ID NO: 6760; SEQ ID NO: 6761; SEQ ID NO: 6762; SEQ ID NO: 6763; SEQ ID NO: 6764; SEQ ID NO: 6765; SEQ ID NO: 6766; SEQ ID NO: 6767 and SEQ ID NO: 9709.

In another embodiment, the H. pylori cytoplasmic polypeptide or a fragment thereof is an H. pylori polypeptide involved in carbohydrate metabolism or a fragment thereof having an amino acid sequence selected from the group consisting of SEQ ID NO: 6768; SEQ ID NO: 6769; SEQ ID NO: 6770; SEQ ID NO: 6771; SEQ ID NO: 6772; SEQ ID NO: 6773; SEQ ID NO: 6774; SEQ ID NO: 6775; SEQ ID NO: 6776; SEQ ID NO: 6777; SEQ ID NO: 6778; SEQ ID NO: 6779; SEQ ID NO: 6780; SEQ ID NO: 6781; SEQ ID NO: 6782; SEQ ID NO: 6783; SEQ ID NO: 6784; SEQ ID NO: 6785; SEQ ID NO: 6786; SEQ ID NO: 6787; SEQ ID NO: 6788; SEQ ID NO: 6789; SEQ ID NO: 6790; SEQ ID NO: 6791; SEQ ID NO: 6792; SEQ ID NO: 6793; SEQ ID NO: 6794; SEQ ID NO: 6795; SEQ ID NO: 6796; SEQ ID NO: 6797; SEQ ID NO: 6798; SEQ ID NO: 6799; SEQ ID NO: 6800; SEQ ID NO: 6801; SEQ ID NO: 6802; SEQ ID NO: 6803; SEQ ID NO: 6804; SEQ ID NO: 6805; SEQ ID NO: 6806; SEQ ID NO: 6807; SEQ ID NO: 6808; SEQ ID NO: 6809; SEQ ID NO: 6810; SEQ ID NO: 6811; SEQ ID NO: 6812; SEQ ID NO: 6813; SEQ ID NO: 6814; SEQ ID NO: 6815; SEQ ID NO: 6816; SEQ ID NO: 6817; SEQ ID NO: 6818; SEQ ID NO: 6819; SEQ ID NO: 6820; SEQ ID NO: 6821; SEQ ID NO: 6822; SEQ ID NO: 6823; SEQ ID NO: 6824; SEQ ID NO: 6825; SEQ ID NO: 6826; SEQ ID NO: 6827; SEQ ID NO: 6828; SEQ ID NO: 6829; SEQ ID NO: 6830; SEQ ID NO: 6831; SEQ ID NO: 6832; SEQ ID NO: 6833; SEQ ID NO: 6834; SEQ ID NO: 6835; SEQ ID NO: 6836; SEQ ID NO: 6837; SEQ ID NO: 6838; SEQ ID NO: 9710 and SEQ ID NO: 9711.

In another embodiment, the H. pylori cytoplasmic polypeptide or a fragment thereof is an H. pylori polypeptide involved in nucleic acid metabolism or a fragment thereof having an amino acid sequence selected from the group consisting of SEQ ID NO: 6839; SEQ ID NO: 6840; SEQ ID NO: 6841; SEQ ID NO: 6842; SEQ ID NO: 6843; SEQ ID NO: 6844; SEQ ID NO: 6845; SEQ ID NO: 6846; SEQ ID NO: 6847; SEQ ID NO: 6848; SEQ ID NO: 6849; SEQ ID NO: 6850; SEQ ID NO: 6851; SEQ ID NO: 6852; SEQ ID NO: 6853; SEQ ID NO: 6854; SEQ ID NO: 6855; SEQ ID NO: 6856; SEQ ID NO: 6857; SEQ ID NO: 6858; SEQ ID NO: 6859; SEQ ID NO: 6860; SEQ ID NO: 6861; SEQ ID NO: 6862; SEQ ID NO: 6863; SEQ ID NO: 6864; SEQ ID NO: 6865; SEQ ID NO: 6866; SEQ ID NO: 6867; SEQ ID NO: 6868; SEQ ID NO: 6869; SEQ ID NO: 6870; SEQ ID NO: 6871; SEQ ID NO: 6872; SEQ ID NO: 6873; SEQ ID NO: 6874; SEQ ID NO: 6875; SEQ ID NO: 6876; SEQ ID NO: 6877; SEQ ID NO: 6878; SEQ ID NO: 6879; SEQ ID NO: 6880; SEQ ID NO: 6881; SEQ ID NO: 6882; SEQ ID NO: 6883; SEQ ID NO: 6884; SEQ ID NO: 6885; SEQ ID NO: 6886; SEQ ID NO: 6887; SEQ ID NO: 6888; SEQ ID NO: 6889; SEQ ID NO: 6890; SEQ ID NO: 6891; SEQ ID NO: 6892; SEQ ID NO: 6893; SEQ ID NO: 6894; SEQ ID NO: 6895; SEQ ID NO: 6896; SEQ ID NO: 6897; SEQ ID NO: 6898; SEQ ID NO: 6899; SEQ ID NO: 6900; SEQ ID NO: 6901; SEQ ID NO: 6902; SEQ ID NO: 6903; SEQ ID NO: 6904; SEQ ID NO: 6905; SEQ ID NO: 6906; SEQ ID NO: 6907; SEQ ID NO: 6908; SEQ ID NO: 6909; SEQ ID NO: 6910; SEQ ID NO: 6911; SEQ ID NO: 6912; SEQ ID NO: 6913; SEQ ID NO: 6914; SEQ ID NO: 6915; SEQ ID NO: 6916; SEQ ID NO: 6917; SEQ ID NO: 6918; SEQ ID NO: 6919; SEQ ID NO: 6920; SEQ ID NO: 6921; SEQ ID NO: 6922; SEQ ID NO: 6923; SEQ ID NO: 6924; SEQ ID NO: 6925; SEQ ID NO: 6926; SEQ ID NO: 6927; SEQ ID NO: 6928; SEQ ID NO: 6929; SEQ ID NO: 6930; SEQ ID NO: 6931; SEQ ID NO: 6932; SEQ ID NO: 6933; SEQ ID NO: 6934; SEQ ID NO: 6935; SEQ ID NO: 6936; SEQ ID NO: 6937; SEQ ID NO: 6938; SEQ ID NO: 6939; SEQ ID NO: 6940; SEQ ID NO: 6941; SEQ ID NO: 6942; SEQ ID NO: 6943; SEQ ID NO: 6944 and SEQ ID NO: 9712.

In another embodiment, the H. pylori cytoplasmic polypeptide or a fragment thereof is an H. pylori polypeptide involved in lipid metabolism or a fragment thereof having an amino acid sequence selected from the group consisting of SEQ ID NO: 6945; SEQ ID NO: 6946; SEQ ID NO: 6947; SEQ ID NO: 6948; SEQ ID NO: 6949; SEQ ID NO: 6950; SEQ ID NO: 6951; SEQ ID NO: 6952; SEQ ID NO: 6953; SEQ ID NO: 6954; SEQ ID NO: 6955; SEQ ID NO: 6956; SEQ ID NO: 6957; SEQ ID NO: 6958; SEQ ID NO: 6959; SEQ ID NO: 6960; SEQ ID NO: 6961; SEQ ID NO: 6962; SEQ ID NO: 6963; SEQ ID NO: 6964; SEQ ID NO: 6965; SEQ ID NO: 6966; SEQ ID NO: 6967; SEQ ID NO: 6968; SEQ ID NO: 6969; SEQ ID NO: 6970; SEQ ID NO: 6971; SEQ ID NO: 6972; SEQ ID NO: 6973; SEQ ID NO: 6974; SEQ ID NO: 6975; SEQ ID NO: 6976; SEQ ID NO: 6977; SEQ ID NO: 6978; SEQ ID NO: 6979; SEQ ID NO: 6980; SEQ ID NO: 9713 and SEQ ID NO: 9714.

In another embodiment, the H. pylori cytoplasmic polypeptide or a fragment thereof is an H. pylori polypeptide involved in mRNA translation and ribosome biogenesis or a fragment thereof having an amino acid sequence selected from the group consisting of SEQ ID NO: 6981; SEQ ID NO: 6982; SEQ ID NO: 6983; SEQ ID NO: 6984; SEQ ID NO: 6985; SEQ ID NO: 6986; SEQ ID NO: 6987; SEQ ID NO: 6988; SEQ ID NO: 6989; SEQ ID NO: 6990; SEQ ID NO: 6991; SEQ ID NO: 6992; SEQ ID NO: 6993; SEQ ID NO: 6994; SEQ ID NO: 6995; SEQ ID NO: 6996; SEQ ID NO: 6997; SEQ ID NO: 6998; SEQ ID NO: 6999; SEQ ID NO: 7000; SEQ ID NO: 7001; SEQ ID NO: 7002; SEQ ID NO: 7003; SEQ ID NO: 7004; SEQ ID NO: 7005; SEQ ID NO: 7006; SEQ ID NO: 7007; SEQ ID NO: 7008; SEQ ID NO: 7009; SEQ ID NO: 7010; SEQ ID NO: 7011; SEQ ID NO: 7012; SEQ ID NO: 7013; SEQ ID NO: 7014; SEQ ID NO: 7015; SEQ ID NO: 7016; SEQ ID NO: 7017; SEQ ID NO: 7018; SEQ ID NO: 7019; SEQ ID NO: 7020; SEQ ID NO: 7021; SEQ ID NO: 7022; SEQ ID NO: 7023; SEQ ID NO: 7024; SEQ ID NO: 7025; SEQ ID NO: 7026; SEQ ID NO: 7027; SEQ ID NO: 7028; SEQ ID NO: 7029; SEQ ID NO: 7030; SEQ ID NO: 7031; SEQ ID NO: 7032; SEQ ID NO: 7033; SEQ ID NO: 7034; SEQ ID NO: 7035; SEQ ID NO: 7036; SEQ ID NO: 7037; SEQ ID NO: 7038; SEQ ID NO: 7039; SEQ ID NO: 7040; SEQ ID NO: 7041; SEQ ID NO: 7042; SEQ ID NO: 7043; SEQ ID NO: 7044; SEQ ID NO: 7045; SEQ ID NO: 7046; SEQ ID NO: 7047; SEQ ID NO: 7048; SEQ ID NO: 7049; SEQ ID NO: 7050; SEQ ID NO: 7051; SEQ ID NO: 7052; SEQ ID NO: 7053; SEQ ID NO: 7054; SEQ ID NO: 7055; SEQ ID NO: 7056; SEQ ID NO: 7057; SEQ ID NO: 7058; SEQ ID NO: 7059; SEQ ID NO: 7060; SEQ ID NO: 7061; SEQ ID NO: 7062; SEQ ID NO: 7063; SEQ ID NO: 7064; SEQ ID NO: 7065; SEQ ID NO: 7066; SEQ ID NO: 7067; SEQ ID NO: 7068; SEQ ID NO: 7069; SEQ ID NO: 7070; SEQ ID NO: 7071; SEQ ID NO: 7072; SEQ ID NO: 7073; SEQ ID NO: 7074; SEQ ID NO: 7075; SEQ ID NO: 7076; SEQ ID NO: 7077; SEQ ID NO: 7078; SEQ ID NO: 7079; SEQ ID NO: 7080; SEQ ID NO: 7081; SEQ ID NO: 7082; SEQ ID NO: 7083; SEQ ID NO: 7084; SEQ ID NO: 7085; SEQ ID NO: 7086; SEQ ID NO: 7087; SEQ ID NO: 7088; SEQ ID NO: 7089; SEQ ID NO: 7090; SEQ ID NO: 7091; SEQ ID NO: 7092; SEQ ID NO: 7093; SEQ ID NO: 7094; SEQ ID NO: 7095; SEQ ID NO: 7096; SEQ ID NO: 7097; SEQ ID NO: 7098; SEQ ID NO: 7099; SEQ ID NO: 7100; SEQ ID NO: 7101; SEQ ID NO: 7102; SEQ ID NO: 7103; SEQ ID NO: 7104; SEQ ID NO: 7105; SEQ ID NO: 7106; SEQ ID NO: 7107; SEQ ID NO: 7108; SEQ ID NO: 7109; SEQ ID NO: 7110; SEQ ID NO: 7111; SEQ ID NO: 7112; SEQ ID NO: 7113; SEQ ID NO: 7114; SEQ ID NO: 7115; SEQ ID NO: 7116; SEQ ID NO: 7117; SEQ ID NO: 7118; SEQ ID NO: 7119; SEQ ID NO: 7120; SEQ ID NO: 7121; SEQ ID NO: 7122; SEQ ID NO: 7123; SEQ ID NO: 7124; SEQ ID NO: 7125; SEQ ID NO: 7126; SEQ ID NO: 7127; SEQ ID NO: 7128; SEQ ID NO: 7129; SEQ ID NO: 7130; SEQ ID NO: 7131; SEQ ID NO: 7132; SEQ ID NO: 7133; SEQ ID NO: 7134; SEQ ID NO: 7135; SEQ ID NO: 7136; SEQ ID NO: 7137; SEQ ID NO: 7138; SEQ ID NO: 7139; SEQ ID NO: 7140; SEQ ID NO: 7141; SEQ ID NO: 7142; SEQ ID NO: 7143; SEQ ID NO: 7144; SEQ ID NO: 7145; SEQ ID NO: 7146; SEQ ID NO: 7147; SEQ ID NO: 7148; SEQ ID NO: 7149; SEQ ID NO: 7150; SEQ ID NO: 7151; SEQ ID NO: 7152; SEQ ID NO: 7153; SEQ ID NO: 7154; SEQ ID NO: 7155; SEQ ID NO: 7156; SEQ ID NO: 7157; SEQ ID NO: 7158; SEQ ID NO: 7159; SEQ ID NO: 7160; SEQ ID NO: 7161; SEQ ID NO: 7162; SEQ ID NO: 7163; SEQ ID NO: 7164; SEQ ID NO: 7165; SEQ ID NO: 7166; SEQ ID NO: 7167; SEQ ID NO: 7168; SEQ ID NO: 7169; SEQ ID NO: 7170; SEQ ID NO: 7171; SEQ ID NO: 7172; SEQ ID NO: 7173; SEQ ID NO: 7174; SEQ ID NO: 7175; SEQ ID NO: 7176; SEQ ID NO: 7177; SEQ ID NO: 7178; SEQ ID NO: 7179; SEQ ID NO: 7180; SEQ ID NO: 7181; SEQ ID NO: 7182; SEQ ID NO: 7183; SEQ ID NO: 7184; SEQ ID NO: 7185; SEQ ID NO: 7186; SEQ ID NO: 7187; SEQ ID NO: 7188; SEQ ID NO: 7189; SEQ ID NO: 7190; SEQ ID NO: 7191; SEQ ID NO: 7192; SEQ ID NO: 7193; SEQ ID NO: 7194; SEQ ID NO: 7195; SEQ ID NO: 7196; SEQ ID NO: 7197; SEQ ID NO: 7198; SEQ ID NO: 7199; SEQ ID NO: 7200; SEQ ID NO: 9715 and SEQ ID NO: 9716.

In another embodiment, the H. pylori cytoplasmic polypeptide or a fragment thereof is an H. pylori polypeptide involved in genome replication, transcription, recombination and repair or a fragment thereof having an amino acid sequence selected from the group consisting of SEQ ID NO: 7201; SEQ ID NO: 7202; SEQ ID NO: 7203; SEQ ID NO: 7204; SEQ ID NO: 7205; SEQ ID NO: 7206; SEQ ID NO: 7207; SEQ ID NO: 7208; SEQ ID NO: 7209; SEQ ID NO: 7210; SEQ ID NO: 7211; SEQ ID NO: 7212; SEQ ID NO: 7213; SEQ ID NO: 7214; SEQ ID NO: 7215; SEQ ID NO: 7216; SEQ ID NO: 7217; SEQ ID NO: 7218; SEQ ID NO: 7219; SEQ ID NO: 7220; SEQ ID NO: 7221; SEQ ID NO: 7222; SEQ ID NO: 7223; SEQ ID NO: 7224; SEQ ID NO: 7225; SEQ ID NO: 7226; SEQ ID NO: 7227; SEQ ID NO: 7228; SEQ ID NO: 7229; SEQ ID NO: 7230; SEQ ID NO: 7231; SEQ ID NO: 7232; SEQ ID NO: 7233; SEQ ID NO: 7234; SEQ ID NO: 7235; SEQ ID NO: 7236; SEQ ID NO: 7237; SEQ ID NO: 7238; SEQ ID NO: 7239; SEQ ID NO: 7240; SEQ ID NO: 7241; SEQ ID NO: 7242; SEQ ID NO: 7243; SEQ ID NO: 7244; SEQ ID NO: 7245; SEQ ID NO: 7246; SEQ ID NO: 7247; SEQ ID NO: 7248; SEQ ID NO: 7249; SEQ ID NO: 7250; SEQ ID NO: 7251; SEQ ID NO: 7252; SEQ ID NO: 7253; SEQ ID NO: 7254; SEQ ID NO: 7255; SEQ ID NO: 7256; SEQ ID NO: 7257; SEQ ID NO: 7258; SEQ ID NO: 7259; SEQ ID NO: 7260; SEQ ID NO: 7261; SEQ ID NO: 7262; SEQ ID NO: 7263; SEQ ID NO: 7264; SEQ ID NO: 7265; SEQ ID NO: 7266; SEQ ID NO: 7267; SEQ ID NO: 7268; SEQ ID NO: 7269; SEQ ID NO: 7270; SEQ ID NO: 7271; SEQ ID NO: 7272; SEQ ID NO: 7273; SEQ ID NO: 7274; SEQ ID NO: 7275; SEQ ID NO: 7276; SEQ ID NO: 7277; SEQ ID NO: 7278; SEQ ID NO: 7279; SEQ ID NO: 7280; SEQ ID NO: 7281; SEQ ID NO: 7282; SEQ ID NO: 7283; SEQ ID NO: 7284; SEQ ID NO: 7285; SEQ ID NO: 7286; SEQ ID NO: 7287; SEQ ID NO: 7288; SEQ ID NO: 7289; SEQ ID NO: 7290; SEQ ID NO: 7291; SEQ ID NO: 7292; SEQ ID NO: 7293; SEQ ID NO: 7294; SEQ ID NO: 7295; SEQ ID NO: 7296; SEQ ID NO: 7297; SEQ ID NO: 7298; SEQ ID NO: 7299; SEQ ID NO: 7300; SEQ ID NO: 7301; SEQ ID NO: 7302; SEQ ID NO: 7303; SEQ ID NO: 7304; SEQ ID NO: 7305; SEQ ID NO: 7306; SEQ ID NO: 7307; SEQ ID NO: 7308; SEQ ID NO: 7309; SEQ ID NO: 7310; SEQ ID NO: 7311; SEQ ID NO: 7312; SEQ ID NO: 7313; SEQ ID NO: 7314; SEQ ID NO: 7315; SEQ ID NO: 7316; SEQ ID NO: 7317; SEQ ID NO: 7318; SEQ ID NO: 7319; SEQ ID NO: 7320; SEQ ID NO: 7321; SEQ ID NO: 7322; SEQ ID NO: 7323; SEQ ID NO: 7324; SEQ ID NO: 7325; SEQ ID NO: 7326; SEQ ID NO: 7327; SEQ ID NO: 7328; SEQ ID NO: 7329; SEQ ID NO: 7330; SEQ ID NO: 7331; SEQ ID NO: 7332; SEQ ID NO: 7333; SEQ ID NO: 7334; SEQ ID NO: 7335; SEQ ID NO: 7336; SEQ ID NO: 7337; SEQ ID NO: 7338; SEQ ID NO: 7339; SEQ ID NO: 7340; SEQ ID NO: 7341; SEQ ID NO: 7342; SEQ ID NO: 7343; SEQ ID NO: 7344; SEQ ID NO: 7345; SEQ ID NO: 7346; SEQ ID NO: 7347; SEQ ID NO: 7348; SEQ ID NO: 7349; SEQ ID NO: 7350; SEQ ID NO: 7351; SEQ ID NO: 7352; SEQ ID NO: 7353; SEQ ID NO: 7354; SEQ ID NO: 7355; SEQ ID NO: 7356; SEQ ID NO: 7357; SEQ ID NO: 7358; SEQ ID NO: 7359; SEQ ID NO: 7360; SEQ ID NO: 7361; SEQ ID NO: 7362; SEQ ID NO: 7363; SEQ ID NO: 7364; SEQ ID NO: 7365; SEQ ID NO: 7366; SEQ ID NO: 7367; SEQ ID NO: 7368; SEQ ID NO: 7369; SEQ ID NO: 7370; SEQ ID NO: 7371; SEQ ID NO: 7372; SEQ ID NO: 7373; SEQ ID NO: 7374; SEQ ID NO: 7375; SEQ ID NO: 7376; SEQ ID NO: 7377; SEQ ID NO: 7378; SEQ ID NO: 7379; SEQ ID NO: 7380; SEQ ID NO: 7381; SEQ ID NO: 7382; SEQ ID NO: 7383; SEQ ID NO: 7384; SEQ ID NO: 7385; SEQ ID NO: 7386; SEQ ID NO: 7387; SEQ ID NO: 7388; SEQ ID NO: 7389; SEQ ID NO: 7390; SEQ ID NO: 7391; SEQ ID NO: 7392; SEQ ID NO: 7393; SEQ ID NO: 7394; SEQ ID NO: 7395; SEQ ID NO: 7396; SEQ ID NO: 7397; SEQ ID NO: 7398; SEQ ID NO: 7399; SEQ ID NO: 7400; SEQ ID NO: 7401; SEQ ID NO: 7402; SEQ ID NO: 7403; SEQ ID NO: 7404; SEQ ID NO: 7405; SEQ ID NO: 7406; SEQ ID NO: 7407; SEQ ID NO: 7408; SEQ ID NO: 7409; SEQ ID NO: 7410; SEQ ID NO: 7411; SEQ ID NO: 7412; SEQ ID NO: 7413; SEQ ID NO: 7414; SEQ ID NO: 7415; SEQ ID NO: 7416; SEQ ID NO: 7417; SEQ ID NO: 7418; SEQ ID NO: 7419; SEQ ID NO: 7420; SEQ ID NO: 7421; SEQ ID NO: 7422; SEQ ID NO: 7423; SEQ ID NO: 7424; SEQ ID NO: 7425; SEQ ID NO: 7426; SEQ ID NO: 7427; SEQ ID NO: 7428; SEQ ID NO: 7429; SEQ ID NO: 7430; SEQ ID NO: 7431; SEQ ID NO: 7432; SEQ ID NO: 7433; SEQ ID NO: 7434; SEQ ID NO: 7435; SEQ ID NO: 7436; SEQ ID NO: 7437; SEQ ID NO: 7438; SEQ ID NO: 7439; SEQ ID NO: 7440; SEQ ID NO: 7441; SEQ ID NO: 7442; SEQ ID NO: 7443; SEQ ID NO: 7444; SEQ ID NO: 7445; SEQ ID NO: 7446; SEQ ID NO: 7447; SEQ ID NO: 7448; SEQ ID NO: 7449; SEQ ID NO: 7450; SEQ ID NO: 7451; SEQ ID NO: 7452; SEQ ID NO: 7453; SEQ ID NO: 7454; SEQ ID NO: 7455; SEQ ID NO: 7456; SEQ ID NO: 7457; SEQ ID NO: 7458; SEQ ID NO: 7459; SEQ ID NO: 7460; SEQ ID NO: 7461; SEQ ID NO: 7462; SEQ ID NO: 7463; SEQ ID NO: 7464; SEQ ID NO: 7465; SEQ ID NO: 7466; SEQ ID NO: 7467; SEQ ID NO: 7468; SEQ ID NO: 7469; SEQ ID NO: 7470; SEQ ID NO: 7471; SEQ ID NO: 7472; SEQ ID NO: 7473; SEQ ID NO: 7474; SEQ ID NO: 7475; SEQ ID NO: 7476; SEQ ID NO: 7477; SEQ ID NO: 7478; SEQ ID NO: 7479; SEQ ID NO: 7480; SEQ ID NO: 7481; SEQ ID NO: 7482; SEQ ID NO: 7483; SEQ ID NO: 7484; SEQ ID NO: 7485; SEQ ID NO: 7486; SEQ ID NO: 7487; SEQ ID NO: 7488; SEQ ID NO: 7489; SEQ ID NO: 7490; SEQ ID NO: 7491; SEQ ID NO: 7492; SEQ ID NO: 7493; SEQ ID NO: 7494; SEQ ID NO: 7495; SEQ ID NO: 7496; SEQ ID NO: 7497; SEQ ID NO: 7498; SEQ ID NO: 7499; SEQ ID NO: 7500; SEQ ID NO: 7501; SEQ ID NO: 7502; SEQ ID NO: 7503; SEQ ID NO: 7504; SEQ ID NO: 7505; SEQ ID NO: 7506; SEQ ID NO: 7507; SEQ ID NO: 7508; SEQ ID NO: 7509; SEQ ID NO:7510; SEQ ID NO: 7511; SEQ ID NO: 7512; SEQ ID NO: 7513; SEQ ID NO: 7514; SEQ ID NO: 7515; SEQ ID NO: 7516; SEQ ID NO: 7517; SEQ ID NO: 7518; SEQ ID NO: 7519; SEQ ID NO: 7520; SEQ ID NO: 7521; SEQ ID NO: 7522; SEQ ID NO: 7523; SEQ ID NO: 7524; SEQ ID NO: 7525; SEQ ID NO: 7526; SEQ ID NO: 7527; SEQ ID NO: 7528; SEQ ID NO: 7529; SEQ ID NO: 7530; SEQ ID NO: 7531; SEQ ID NO: 7532; SEQ ID NO: 7533; SEQ ID NO: 7534; SEQ ID NO: 7535; SEQ ID NO: 7536; SEQ ID NO: 7537; SEQ ID NO: 7538; SEQ ID NO: 7539; SEQ ID NO: 7540; SEQ ID NO: 7541; SEQ ID NO: 7542; SEQ ID NO: 7543; SEQ ID NO: 7544; SEQ ID NO: 7545; SEQ ID NO: 7546; SEQ ID NO: 7547; SEQ ID NO: 7548; SEQ ID NO: 7549; SEQ ID NO: 7550; SEQ ID NO: 7551; SEQ ID NO: 7552; SEQ ID NO: 7553; SEQ ID NO: 7554; SEQ ID NO: 7555; SEQ ID NO: 7556; SEQ ID NO: 7557; SEQ ID NO: 7558; SEQ ID NO: 7559; SEQ ID NO: 7560; SEQ ID NO: 7561; SEQ ID NO: 7562; SEQ ID NO: 7563; SEQ ID NO: 7564; SEQ ID NO: 7565; SEQ ID NO: 7566; SEQ ID NO: 7567; SEQ ID NO: 7568; SEQ ID NO: 7569; SEQ ID NO: 9717; SEQ ID NO: 9718; SEQ ID NO: 9719 and SEQ ID NO: 9720.

In another embodiment, the H. pylori cytoplasmic polypeptide or a fragment thereof is an H. pylori polypeptide involved in secretion and adhesion or a fragment thereof having an amino acid sequence selected from the group consisting of SEQ ID NO: 7570; SEQ ID NO: 7571; SEQ ID NO: 7572; SEQ ID NO: 7573; SEQ ID NO: 7574; SEQ ID NO: 7575; SEQ ID NO: 7576; SEQ ID NO: 7577; SEQ ID NO: 7578; SEQ ID NO: 7579; SEQ ID NO: 7580; SEQ ID NO: 7581; SEQ ID NO: 7582; SEQ ID NO: 7583; SEQ ID NO: 7584; SEQ ID NO: 7585; SEQ ID NO: 7586.

In another embodiment, the H. pylori cytoplasmic polypeptide or a fragment thereof is an H. pylori polypeptide involved in outer membrane and cell wall biogenesis or a fragment thereof having an amino acid sequence selected from the group consisting of SEQ ID NO: 7587; SEQ ID NO: 7588; SEQ ID NO: 7589; SEQ ID NO: 7590; SEQ ID NO: 7591; SEQ ID NO: 7592; SEQ ID NO: 7593; SEQ ID NO: 7594; SEQ ID NO: 7595; SEQ ID NO: 7596; SEQ ID NO: 7597; SEQ ID NO: 7598; SEQ ID NO: 7599; SEQ ID NO: 7600; SEQ ID NO: 7601; SEQ ID NO: 7602; SEQ ID NO: 7603; SEQ ID NO: 7604; SEQ ID NO: 7605; SEQ ID NO: 7606; SEQ ID NO: 7607; SEQ ID NO: 7608; SEQ ID NO: 7609; SEQ ID NO: 7610; SEQ ID NO: 7611; SEQ ID NO: 7612; SEQ ID NO: 7613; SEQ ID NO: 7614; SEQ ID NO: 7615; SEQ ID NO: 7616; SEQ ID NO: 7617; SEQ ID NO: 7618; SEQ ID NO: 7619; SEQ ID NO: 7620; SEQ ID NO: 7621; SEQ ID NO: 7622; SEQ ID NO: 7623; SEQ ID NO: 7624; SEQ ID NO: 7625; SEQ ID NO: 7626; SEQ ID NO: 7627; SEQ ID NO: 7628; SEQ ID NO: 7629; SEQ ID NO: 7630; SEQ ID NO: 7631; SEQ ID NO: 7632; SEQ ID NO: 7633; SEQ ID NO: 7634; SEQ ID NO: 7635; SEQ ID NO: 7636; SEQ ID NO: 7637; SEQ ID NO: 7638; SEQ ID NO: 7639; SEQ ID NO: 9721 and SEQ ID NO: 9722.

In another embodiment, the H. pylori cytoplasmic polypeptide or a fragment thereof is an H. pylori polypeptide involved in protein folding and stabilization or a fragment thereof having an amino acid sequence selected from the group consisting of SEQ ID NO: 7640; SEQ ID NO: 7641; SEQ ID NO: 7642; SEQ ID NO: 7643; SEQ ID NO: 7644; SEQ ID NO: 7645; SEQ ID NO: 7646; SEQ ID NO: 7647; SEQ ID NO: 7648; SEQ ID NO: 7649; SEQ ID NO: 7650; SEQ ID NO: 7651; SEQ ID NO: 7652; SEQ ID NO: 7653; SEQ ID NO: 7654; SEQ ID NO: 7655; SEQ ID NO: 7656; SEQ ID NO: 7657; SEQ ID NO: 7658; SEQ ID NO: 7659; SEQ ID NO: 7660; SEQ ID NO: 7661; SEQ ID NO: 7662; SEQ ID NO: 7663; SEQ ID NO: 7664; SEQ ID NO: 7665; SEQ ID NO: 7666; SEQ ID NO: 7667; SEQ ID NO: 7668; SEQ ID NO: 7669; SEQ ID NO: 7670; SEQ ID NO: 7671; SEQ ID NO: 7672; SEQ ID NO: 7673; SEQ ID NO: 7674; SEQ ID NO: 7675; SEQ ID NO: 7676; SEQ ID NO: 7677; SEQ ID NO: 7678; SEQ ID NO: 7679; SEQ ID NO: 7680; SEQ ID NO: 9723 and SEQ ID NO: 9724.

Particularly preferred is an isolated H. pylori cytoplasmic polypeptide or a fragment thereof, wherein the polypeptide is encoded by a nucleic acid sequence selected from the group consisting of SEQ ID NO: 1576; SEQ ID NO: 1577; SEQ ID NO: 1578; SEQ ID NO: 1579; SEQ ID NO: 1580; SEQ ID NO: 1581; SEQ ID NO: 1582; SEQ ID NO: 1583; SEQ ID NO: 1584; SEQ ID NO: 1585; SEQ ID NO: 1586; SEQ ID NO: 1587; SEQ ID NO: 1588; SEQ ID NO: 1589; SEQ ID NO: 1590; SEQ ID NO: 1591; SEQ ID NO: 1592; SEQ ID NO: 1593; SEQ ID NO: 1594; SEQ ID NO: 1595; SEQ ID NO: 1596; SEQ ID NO: 1597; SEQ ID NO: 1598; SEQ ID NO: 1599; SEQ ID NO: 1600; SEQ ID NO: 1601; SEQ ID NO: 1602; SEQ ID NO: 1603; SEQ ID NO: 1604; SEQ ID NO: 1605; SEQ ID NO: 1606; SEQ ID NO: 1607; SEQ ID NO: 1608; SEQ ID NO: 1609; SEQ ID NO: 1610; SEQ ID NO: 1611; SEQ ID NO: 1612; SEQ ID NO: 1613; SEQ ID NO: 1614; SEQ ID NO: 1615; SEQ ID NO: 1616; SEQ ID NO: 1617; SEQ ID NO: 1618; SEQ ID NO: 1619; SEQ ID NO: 1620; SEQ ID NO: 1621; SEQ ID NO: 1622; SEQ ID NO: 1623; SEQ ID NO: 1624; SEQ ID NO: 1625; SEQ ID NO: 1626; SEQ ID NO: 1627; SEQ ID NO: 1628; SEQ ID NO: 1629; SEQ ID NO: 1630; SEQ ID NO: 1631; SEQ ID NO: 1632; SEQ ID NO: 1633; SEQ ID NO: 1634; SEQ ID NO: 1635; SEQ ID NO: 1636; SEQ ID NO: 1637; SEQ ID NO: 1638; SEQ ID NO: 1639; SEQ ID NO: 1640; SEQ ID NO: 1641; SEQ ID NO: 1642; SEQ ID NO: 1643; SEQ ID NO: 1644; SEQ ID NO: 1645; SEQ ID NO: 1646; SEQ ID NO: 1647; SEQ ID NO: 1648; SEQ ID NO: 1649; SEQ ID NO: 1650; SEQ ID NO: 1651; SEQ ID NO: 1652; SEQ ID NO: 1653; SEQ ID NO: 1654; SEQ ID NO: 1655; SEQ ID NO: 1656; SEQ ID NO: 1657; SEQ ID NO: 1658; SEQ ID NO: 1659; SEQ ID NO: 1660; SEQ ID NO: 1661; SEQ ID NO: 1662; SEQ ID NO: 1663; SEQ ID NO: 1664; SEQ ID NO: 1665; SEQ ID NO: 1666; SEQ ID NO: 1667; SEQ ID NO: 1668; SEQ ID NO: 1669; SEQ ID NO: 1670; SEQ ID NO: 1671; SEQ ID NO: 1672; SEQ ID NO: 1673; SEQ ID NO: 1674; SEQ ID NO: 1675; SEQ ID NO: 1676; SEQ ID NO: 1677; SEQ ID NO: 1678; SEQ ID NO: 1679; SEQ ID NO: 1680; SEQ ID NO: 1681; SEQ ID NO: 1682; SEQ ID NO: 1683; SEQ ID NO: 1684; SEQ ID NO: 1685; SEQ ID NO: 1686; SEQ ID NO: 1687; SEQ ID NO: 1688; SEQ ID NO: 1689; SEQ ID NO: 1690; SEQ ID NO: 1691; SEQ ID NO: 1692; SEQ ID NO: 1693; SEQ ID NO: 1694; SEQ ID NO: 1695; SEQ ID NO: 1696; SEQ ID NO: 1697; SEQ ID NO: 1698; SEQ ID NO: 1699; SEQ ID NO: 1700; SEQ ID NO: 1701; SEQ ID NO: 1702; SEQ ID NO: 1703; SEQ ID NO: 1704; SEQ ID NO: 1705; SEQ ID NO: 1706; SEQ ID NO: 1707; SEQ ID NO: 1708; SEQ ID NO: 1709; SEQ ID NO: 1710; SEQ ID NO: 1711; SEQ ID NO: 1712; SEQ ID NO: 1713; SEQ ID NO: 1714; SEQ ID NO: 1715; SEQ ID NO: 1716; SEQ ID NO: 1717; SEQ ID NO: 1718; SEQ ID NO: 1719; SEQ ID NO: 1720; SEQ ID NO: 1721; SEQ ID NO: 1722; SEQ ID NO: 1723; SEQ ID NO: 1724; SEQ ID NO: 1725; SEQ ID NO: 1726; SEQ ID NO: 1727; SEQ ID NO: 1728; SEQ ID NO: 1729; SEQ ID NO: 1730; SEQ ID NO: 1731; SEQ ID NO: 1732; SEQ ID NO: 1733; SEQ ID NO: 1734; SEQ ID NO: 1735; SEQ ID NO: 1736; SEQ ID NO: 1737; SEQ ID NO: 1738; SEQ ID NO: 1739; SEQ ID NO: 1740; SEQ ID NO: 1741; SEQ ID NO: 1742; SEQ ID NO: 1743; SEQ ID NO: 1744; SEQ ID NO: 1745; SEQ ID NO: 1746; SEQ ID NO: 1747; SEQ ID NO: 1748; SEQ ID NO: 1749; SEQ ID NO: 1750; SEQ ID NO: 1751; SEQ ID NO: 1752; SEQ ID NO: 1753; SEQ ID NO: 1754; SEQ ID NO: 1755; SEQ ID NO: 1756; SEQ ID NO: 1757; SEQ ID NO: 1758; SEQ ID NO: 1759; SEQ ID NO: 1760; SEQ ID NO: 1761; SEQ ID NO: 1762; SEQ ID NO: 1763; SEQ ID NO: 1764; SEQ ID NO: 1765; SEQ ID NO: 1766; SEQ ID NO: 1767; SEQ ID NO: 1768; SEQ ID NO: 1769; SEQ ID NO: 1770; SEQ ID NO: 1771; SEQ ID NO: 1772; SEQ ID NO: 1773; SEQ ID NO: 1774; SEQ ID NO: 1775; SEQ ID NO: 1776; SEQ ID NO: 1777; SEQ ID NO: 1778; SEQ ID NO: 1779; SEQ ID NO: 1780; SEQ ID NO: 1781; SEQ ID NO: 1782; SEQ ID NO: 1783; SEQ ID NO: 1784; SEQ ID NO: 1785; SEQ ID NO: 1786; SEQ ID NO: 1787; SEQ ID NO: 1788; SEQ ID NO: 1789; SEQ ID NO: 1790; SEQ ID NO: 1791; SEQ ID NO: 1792; SEQ ID NO: 1793; SEQ ID NO: 1794; SEQ ID NO: 1795; SEQ ID NO: 1796; SEQ ID NO: 1797; SEQ ID NO: 1798; SEQ ID NO: 1799; SEQ ID NO: 1800; SEQ ID NO: 1801; SEQ ID NO: 1802; SEQ ID NO: 1803; SEQ ID NO: 1804; SEQ ID NO: 1805; SEQ ID NO: 1806; SEQ ID NO: 1807; SEQ ID NO: 1808; SEQ ID NO: 1809; SEQ ID NO: 1810; SEQ ID NO: 1811; SEQ ID NO: 1812; SEQ ID NO: 1813; SEQ ID NO: 1814; SEQ ID NO: 1815; SEQ ID NO: 1816; SEQ ID NO: 1817; SEQ ID NO: 1818; SEQ ID NO: 1819; SEQ ID NO: 1820; SEQ ID NO: 1821; SEQ ID NO: 1822; SEQ ID NO: 1823; SEQ ID NO: 1824; SEQ ID NO: 1825; SEQ ID NO: 1826; SEQ ID NO: 1827; SEQ ID NO: 1828; SEQ ID NO: 1829; SEQ ID NO: 1830; SEQ ID NO: 1831; SEQ ID NO: 1832; SEQ ID NO: 1833; SEQ ID NO: 1834; SEQ ID NO: 1835; SEQ ID NO: 1836; SEQ ID NO: 1837; SEQ ID NO: 1838; SEQ ID NO: 1839; SEQ ID NO: 1840; SEQ ID NO: 1841; SEQ ID NO: 1842; SEQ ID NO: 1843; SEQ ID NO: 1844; SEQ ID NO: 1845; SEQ ID NO: 1846; SEQ ID NO: 1847; SEQ ID NO: 1848; SEQ ID NO: 1849; SEQ ID NO: 1850; SEQ ID NO: 1851; SEQ ID NO: 1852; SEQ ID NO: 1853; SEQ ID NO: 1854; SEQ ID NO: 1855; SEQ ID NO: 1856; SEQ ID NO: 1857; SEQ ID NO: 1858; SEQ ID NO: 1859; SEQ ID NO: 1860; SEQ ID NO: 1861; SEQ ID NO: 1862; SEQ ID NO: 1863; SEQ ID NO: 1864; SEQ ID NO: 1865; SEQ ID NO: 1866; SEQ ID NO: 1867; SEQ ID NO: 1868; SEQ ID NO: 1869; SEQ ID NO: 1870; SEQ ID NO: 1871; SEQ ID NO: 1872; SEQ ID NO: 1873; SEQ ID NO: 1874; SEQ ID NO: 1875; SEQ ID NO: 1876; SEQ ID NO: 1877; SEQ ID NO: 1878; SEQ ID NO: 1879; SEQ ID NO: 1880; SEQ ID NO: 1881; SEQ ID NO: 1882; SEQ ID NO: 1883; SEQ ID NO: 1884; SEQ ID NO: 1885; SEQ ID NO: 1886; SEQ ID NO: 1887; SEQ ID NO: 1888; SEQ ID NO: 1889; SEQ ID NO: 1890; SEQ ID NO: 1891; SEQ ID NO: 1892; SEQ ID NO: 1893; SEQ ID NO: 1894; SEQ ID NO: 1895; SEQ ID NO: 1896; SEQ ID NO: 1897; SEQ ID NO: 1898; SEQ ID NO: 1899; SEQ ID NO: 1900; SEQ ID NO: 1901; SEQ ID NO: 1902; SEQ ID NO: 1903; SEQ ID NO: 1904; SEQ ID NO: 1905; SEQ ID NO: 1906; SEQ ID NO: 1907; SEQ ID NO: 1908; SEQ ID NO: 1909; SEQ ID NO: 1910; SEQ ID NO: 1911; SEQ ID NO: 1912; SEQ ID NO: 1913; SEQ ID NO: 1914; SEQ ID NO: 1915; SEQ ID NO: 1916; SEQ ID NO: 1917; SEQ ID NO: 1918; SEQ ID NO: 1919; SEQ ID NO: 1920; SEQ ID NO: 1921; SEQ ID NO: 1922; SEQ ID NO: 1923; SEQ ID NO: 1924; SEQ ID NO: 1925; SEQ ID NO: 1926; SEQ ID NO: 1927; SEQ ID NO: 1928; SEQ ID NO: 1929; SEQ ID NO: 1930; SEQ ID NO: 1931; SEQ ID NO: 1932; SEQ ID NO: 1933; SEQ ID NO: 1934; SEQ ID NO: 1935; SEQ ID NO: 1936; SEQ ID NO: 1937; SEQ ID NO: 1938; SEQ ID NO: 1939; SEQ ID NO: 1940; SEQ ID NO: 1941; SEQ ID NO: 1942; SEQ ID NO: 1943; SEQ ID NO: 1944; SEQ ID NO: 1945; SEQ ID NO: 1946; SEQ ID NO: 1947; SEQ ID NO: 1948; SEQ ID NO: 1949; SEQ ID NO: 1950; SEQ ID NO: 1951; SEQ ID NO: 1952; SEQ ID NO: 1953; SEQ ID NO: 1954; SEQ ID NO: 1955; SEQ ID NO: 1956; SEQ ID NO: 1957; SEQ ID NO: 1958; SEQ ID NO: 1959; SEQ ID NO: 1960; SEQ ID NO: 1961; SEQ ID NO: 1962; SEQ ID NO: 1963; SEQ ID NO: 1964; SEQ ID NO: 1965; SEQ ID NO: 1966; SEQ ID NO: 1967; SEQ ID NO: 1968; SEQ ID NO: 1969; SEQ ID NO: 1970; SEQ ID NO: 1971; SEQ ID NO: 1972; SEQ ID NO: 1973; SEQ ID NO: 1974; SEQ ID NO: 1975; SEQ ID NO: 1976; SEQ ID NO: 1977; SEQ ID NO: 1978; SEQ ID NO: 1979; SEQ ID NO: 1980; SEQ ID NO: 1981; SEQ ID NO: 1982; SEQ ID NO: 1983; SEQ ID NO: 1984; SEQ ID NO: 1985; SEQ ID NO: 1986; SEQ ID NO: 1987; SEQ ID NO: 1988; SEQ ID NO: 1989; SEQ ID NO: 1990; SEQ ID NO: 1991; SEQ ID NO: 1992; SEQ ID NO: 1993; SEQ ID NO: 1994; SEQ ID NO: 1995; SEQ ID NO: 1996; SEQ ID NO: 1997; SEQ ID NO: 1998; SEQ ID NO: 1999; SEQ ID NO: 2000; SEQ ID NO: 2001; SEQ ID NO: 2002; SEQ ID NO: 2003; SEQ ID NO: 2004; SEQ ID NO: 2005; SEQ ID NO: 2006; SEQ ID NO: 2007; SEQ ID NO: 2008; SEQ ID NO: 2009; SEQ ID NO: 2010; SEQ ID NO: 2011; SEQ ID NO: 2012; SEQ ID NO: 2013; SEQ ID NO: 2014; SEQ ID NO: 2015; SEQ ID NO: 2016; SEQ ID NO: 2017; SEQ ID NO: 2018; SEQ ID NO: 2019; SEQ ID NO: 2020; SEQ ID NO: 2021; SEQ ID NO: 2022; SEQ ID NO: 2023; SEQ ID NO: 2024; SEQ ID NO: 2025; SEQ ID NO: 2026; SEQ ID NO: 2027; SEQ ID NO: 2028; SEQ ID NO: 2029; SEQ ID NO: 2030; SEQ ID NO: 2031; SEQ ID NO: 2032; SEQ ID NO: 2033; SEQ ID NO: 2034; SEQ ID NO: 2035; SEQ ID NO: 2036; SEQ ID NO: 2037; SEQ ID NO: 2038; SEQ ID NO: 2039; SEQ ID NO: 2040; SEQ ID NO: 2041; SEQ ID NO: 2042; SEQ ID NO: 2043; SEQ ID NO: 2044; SEQ ID NO: 2045; SEQ ID NO: 2046; SEQ ID NO: 2047; SEQ ID NO: 2048; SEQ ID NO: 2049; SEQ ID NO: 2050; SEQ ID NO: 2051; SEQ ID NO: 2052; SEQ ID NO: 2053; SEQ ID NO: 2054; SEQ ID NO: 2055; SEQ ID NO: 2056; SEQ ID NO: 2057; SEQ ID NO: 2058; SEQ ID NO: 2059; SEQ ID NO: 2060; SEQ ID NO: 2061; SEQ ID NO: 2062; SEQ ID NO: 2063; SEQ ID NO: 2064; SEQ ID NO: 2065; SEQ ID NO: 2066; SEQ ID NO: 2067; SEQ ID NO: 2068; SEQ ID NO: 2069; SEQ ID NO: 2070; SEQ ID NO: 2071; SEQ ID NO: 2072; SEQ ID NO: 2073; SEQ ID NO: 2074; SEQ ID NO: 2075; SEQ ID NO: 2076; SEQ ID NO: 2077; SEQ ID NO: 2078; SEQ ID NO: 2079; SEQ ID NO: 2080; SEQ ID NO: 2081; SEQ ID NO: 2082; SEQ ID NO: 2083; SEQ ID NO: 2084; SEQ ID NO: 2085; SEQ ID NO: 2086; SEQ ID NO: 2087; SEQ ID NO: 2088; SEQ ID NO: 2089; SEQ ID NO: 2090; SEQ ID NO: 2091; SEQ ID NO: 2092; SEQ ID NO: 2093; SEQ ID NO: 2094; SEQ ID NO: 2095; SEQ ID NO: 2096; SEQ ID NO: 2097; SEQ ID NO: 2098; SEQ ID NO: 2099; SEQ ID NO: 2100; SEQ ID NO: 2101; SEQ ID NO: 2102; SEQ ID NO: 2103; SEQ ID NO: 2104; SEQ ID NO: 2105; SEQ ID NO: 2106; SEQ ID NO: 2107; SEQ ID NO: 2108; SEQ ID NO: 2109; SEQ ID NO: 2110; SEQ ID NO: 2111; SEQ ID NO: 2112; SEQ ID NO: 2113; SEQ ID NO: 2114; SEQ ID NO: 2115; SEQ ID NO: 2116; SEQ ID NO: 2117; SEQ ID NO: 2118; SEQ ID NO: 2119; SEQ ID NO: 2120; SEQ ID NO: 2121; SEQ ID NO: 2122; SEQ ID NO: 2123; SEQ ID NO: 2124; SEQ ID NO: 2125; SEQ ID NO: 2126; SEQ ID NO: 2127; SEQ ID NO: 2128; SEQ ID NO: 2129; SEQ ID NO: 2130; SEQ ID NO: 2131; SEQ ID NO: 2132; SEQ ID NO: 2133; SEQ ID NO: 2134; SEQ ID NO: 2135; SEQ ID NO: 2136; SEQ ID NO: 2137; SEQ ID NO: 2138; SEQ ID NO: 2139; SEQ ID NO: 2140; SEQ ID NO: 2141; SEQ ID NO: 2142; SEQ ID NO: 2143; SEQ ID NO: 2144; SEQ ID NO: 2145; SEQ ID NO: 2146; SEQ ID NO: 2147; SEQ ID NO: 2148; SEQ ID NO: 2149; SEQ ID NO: 2150; SEQ ID NO: 2151; SEQ ID NO: 2152; SEQ ID NO: 2153; SEQ ID NO: 2154; SEQ ID NO: 2155; SEQ ID NO: 2156; SEQ ID NO: 2157; SEQ ID NO: 2158; SEQ ID NO: 2159; SEQ ID NO: 2160; SEQ ID NO: 2161; SEQ ID NO: 2162; SEQ ID NO: 2163; SEQ ID NO: 2164; SEQ ID NO: 2165; SEQ ID NO: 2166; SEQ ID NO: 2167; SEQ ID NO: 2168; SEQ ID NO: 2169; SEQ ID NO: 2170; SEQ ID NO: 2171; SEQ ID NO: 2172; SEQ ID NO: 2173; SEQ ID NO: 2174; SEQ ID NO: 2175; SEQ ID NO: 2176; SEQ ID NO: 2177; SEQ ID NO: 2178; SEQ ID NO: 2179; SEQ ID NO: 2180; SEQ ID NO: 2181; SEQ ID NO: 2182; SEQ ID NO: 2183; SEQ ID NO: 2184; SEQ ID NO: 2185; SEQ ID NO: 2186; SEQ ID NO: 2187; SEQ ID NO: 2188; SEQ ID NO: 2189; SEQ ID NO: 2190; SEQ ID NO: 2191; SEQ ID NO: 2192; SEQ ID NO: 2193; SEQ ID NO: 2194; SEQ ID NO: 2195; SEQ ID NO: 2196; SEQ ID NO: 2197; SEQ ID NO: 2198; SEQ ID NO: 2199; SEQ ID NO: 2200; SEQ ID NO: 2201; SEQ ID NO: 2202; SEQ ID NO: 2203; SEQ ID NO: 2204; SEQ ID NO: 2205; SEQ ID NO: 2206; SEQ ID NO: 2207; SEQ ID NO: 2208; SEQ ID NO: 2209; SEQ ID NO: 2210; SEQ ID NO: 2211; SEQ ID NO: 2212; SEQ ID NO: 2213; SEQ ID NO: 2214; SEQ ID NO: 2215; SEQ ID NO: 2216; SEQ ID NO: 2217; SEQ ID NO: 2218; SEQ ID NO: 2219; SEQ ID NO: 2220; SEQ ID NO: 2221; SEQ ID NO: 2222; SEQ ID NO: 2223; SEQ ID NO: 2224; SEQ ID NO: 2225; SEQ ID NO: 2226; SEQ ID NO: 2227; SEQ ID NO: 2228; SEQ ID NO: 2229; SEQ ID NO: 2230; SEQ ID NO: 2231; SEQ ID NO: 2232; SEQ ID NO: 2233; SEQ ID NO: 2234; SEQ ID NO: 2235; SEQ ID NO: 2236; SEQ ID NO: 2237; SEQ ID NO: 2238; SEQ ID NO: 2239; SEQ ID NO: 2240; SEQ ID NO: 2241; SEQ ID NO: 2242; SEQ ID NO: 2243; SEQ ID NO: 2244; SEQ ID NO: 2245; SEQ ID NO: 2246; SEQ ID NO: 2247; SEQ ID NO: 2248; SEQ ID NO: 2249; SEQ ID NO: 2250; SEQ ID NO: 2251; SEQ ID NO: 2252; SEQ ID NO: 2253; SEQ ID NO: 2254; SEQ ID NO: 2255; SEQ ID NO: 2256; SEQ ID NO: 2257; SEQ ID NO: 2258; SEQ ID NO: 2259; SEQ ID NO: 2260; SEQ ID NO: 2261; SEQ ID NO: 2262; SEQ ID NO: 2263; SEQ ID NO: 2264; SEQ ID NO: 2265; SEQ ID NO: 2266; SEQ ID NO: 2267; SEQ ID NO: 2268; SEQ ID NO: 2269; SEQ ID NO: 2270; SEQ ID NO: 2271; SEQ ID NO: 2272; SEQ ID NO: 2273; SEQ ID NO: 2274; SEQ ID NO: 2275; SEQ ID NO: 2276; SEQ ID NO: 2277; SEQ ID NO: 2278; SEQ ID NO: 2279; SEQ ID NO: 2280; SEQ ID NO: 2281; SEQ ID NO: 2282; SEQ ID NO: 2283; SEQ ID NO: 2284; SEQ ID NO: 2285; SEQ ID NO: 2286; SEQ ID NO: 2287; SEQ ID NO: 2288; SEQ ID NO: 2289; SEQ ID NO: 2290; SEQ ID NO: 2291; SEQ ID NO: 2292; SEQ ID NO: 2293; SEQ ID NO: 2294; SEQ ID NO: 2295; SEQ ID NO: 2296; SEQ ID NO: 2297; SEQ ID NO: 2298; SEQ ID NO: 2299; SEQ ID NO: 2300; SEQ ID NO: 2301; SEQ ID NO: 2302; SEQ ID NO: 2303; SEQ ID NO: 2304; SEQ ID NO: 2305; SEQ ID NO: 2306; SEQ ID NO: 2307; SEQ ID NO: 2308; SEQ ID NO: 2309; SEQ ID NO: 2310; SEQ ID NO: 2311; SEQ ID NO: 2312; SEQ ID NO: 2313; SEQ ID NO: 2314; SEQ ID NO: 2315; SEQ ID NO: 2316; SEQ ID NO: 2317; SEQ ID NO: 2318; SEQ ID NO: 2319; SEQ ID NO: 2320; SEQ ID NO: 2321; SEQ ID NO: 2322; SEQ ID NO: 2323; SEQ ID NO: 2324; SEQ ID NO: 2325; SEQ ID NO: 2326; SEQ ID NO:2327; SEQ ID NO: 2328; SEQ ID NO: 2329; SEQ ID NO: 2330; SEQ ID NO: 2331; SEQ ID NO: 2332; SEQ ID NO: 2333; SEQ ID NO: 2334; SEQ ID NO: 2335; SEQ ID NO: 2336; SEQ ID NO: 2337; SEQ ID NO: 2338; SEQ ID NO: 2339; SEQ ID NO: 2340; SEQ ID NO: 2341; SEQ ID NO: 2342; SEQ ID NO: 2343; SEQ ID NO: 2344; SEQ ID NO: 2345; SEQ ID NO: 2346; SEQ ID NO: 2347; SEQ ID NO: 2348; SEQ ID NO: 2349; SEQ ID NO: 2350; SEQ ID NO: 2351; SEQ ID NO: 2352; SEQ ID NO: 2353; SEQ ID NO: 2354; SEQ ID NO: 2355; SEQ ID NO: 2356; SEQ ID NO: 2357; SEQ ID NO: 2358; SEQ ID NO: 2359; SEQ ID NO: 2360; SEQ ID NO: 2361; SEQ ID NO: 2362; SEQ ID NO: 2363; SEQ ID NO: 2364; SEQ ID NO: 2365; SEQ ID NO: 2366; SEQ ID NO: 2367; SEQ ID NO: 2368; SEQ ID NO: 2369; SEQ ID NO: 2370; SEQ ID NO: 2371; SEQ ID NO: 2372; SEQ ID NO: 2373; SEQ ID NO: 2374; SEQ ID NO: 2375; SEQ ID NO: 2376; SEQ ID NO: 2377; SEQ ID NO: 2378; SEQ ID NO: 2379; SEQ ID NO: 2380; SEQ ID NO: 2381; SEQ ID NO: 2382; SEQ ID NO: 2383; SEQ ID NO: 2384; SEQ ID NO: 2385; SEQ ID NO: 2386; SEQ ID NO: 2387; SEQ ID NO: 2388; SEQ ID NO: 2389; SEQ ID NO: 2390; SEQ ID NO: 2391; SEQ ID NO: 2392; SEQ ID NO: 2393; SEQ ID NO: 2394; SEQ ID NO: 2395; SEQ ID NO: 2396; SEQ ID NO: 2397; SEQ ID NO: 2398; SEQ ID NO: 2399; SEQ ID NO: 2400; SEQ ID NO: 2401; SEQ ID NO: 2402; SEQ ID NO: 2403; SEQ ID NO: 2404; SEQ ID NO: 2405; SEQ ID NO: 2406; SEQ ID NO: 2407; SEQ ID NO: 2408; SEQ ID NO: 2409; SEQ ID NO:2410; SEQ ID NO:2411; SEQ ID NO:2412; SEQ ID NO:2413; SEQ ID NO: 2414; SEQ ID NO: 2415; SEQ ID NO: 2416; SEQ ID NO: 2417; SEQ ID NO: 2418; SEQ ID NO: 2419; SEQ ID NO: 2420; SEQ ID NO: 2421; SEQ ID NO: 2422; SEQ ID NO: 2423; SEQ ID NO: 2424; SEQ ID NO: 2425; SEQ ID NO: 2426; SEQ ID NO: 2427; SEQ ID NO: 2428; SEQ ID NO: 2429; SEQ ID NO: 2430; SEQ ID NO: 2431; SEQ ID NO: 2432; SEQ ID NO: 2433; SEQ ID NO: 2434; SEQ ID NO: 2435; SEQ ID NO: 2436; SEQ ID NO: 2437; SEQ ID NO: 2438; SEQ ID NO: 2439; SEQ ID NO: 2440; SEQ ID NO: 2441; SEQ ID NO: 2442; SEQ ID NO: 2443; SEQ ID NO: 2444; SEQ ID NO: 2445; SEQ ID NO: 2446; SEQ ID NO: 2447; SEQ ID NO: 2448; SEQ ID NO: 2449; SEQ ID NO: 2450; SEQ ID NO: 2451; SEQ ID NO: 2452; SEQ ID NO: 2453; SEQ ID NO: 2454; SEQ ID NO: 2455; SEQ ID NO: 2456; SEQ ID NO: 2457; SEQ ID NO: 2458; SEQ ID NO: 2459; SEQ ID NO: 2460; SEQ ID NO: 2461; SEQ ID NO: 2462; SEQ ID NO: 2463; SEQ ID NO: 2464; SEQ ID NO: 2465; SEQ ID NO: 2466; SEQ ID NO: 2467; SEQ ID NO: 2468; SEQ ID NO: 2469; SEQ ID NO: 2470; SEQ ID NO: 2471; SEQ ID NO: 2472; SEQ ID NO: 2473; SEQ ID NO: 2474; SEQ ID NO: 2475; SEQ ID NO: 2476; SEQ ID NO: 2477; SEQ ID NO: 2478; SEQ ID NO: 2479; SEQ ID NO: 2480; SEQ ID NO: 2481; SEQ ID NO: 2482; SEQ ID NO: 2483; SEQ ID NO: 2484; SEQ ID NO: 2485; SEQ ID NO: 2486; SEQ ID NO: 2487; SEQ ID NO: 2488; SEQ ID NO: 2489; SEQ ID NO: 2490; SEQ ID NO: 2491; SEQ ID NO: 2492; SEQ ID NO: 2493; SEQ ID NO: 2494; SEQ ID NO: 2495; SEQ ID NO: 2496; SEQ ID NO: 2497; SEQ ID NO: 2498; SEQ ID NO: 2499; SEQ ID NO: 2500; SEQ ID NO: 2501; SEQ ID NO: 2502; SEQ ID NO: 2503; SEQ ID NO: 2504; SEQ ID NO: 2505; SEQ ID NO: 2506; SEQ ID NO: 2507; SEQ ID NO: 2508; SEQ ID NO: 2509; SEQ ID NO: 2510; SEQ ID NO: 2511; SEQ ID NO: 2512; SEQ ID NO: 2513; SEQ ID NO: 2514; SEQ ID NO: 2515; SEQ ID NO: 2516; SEQ ID NO: 2517; SEQ ID NO: 2518; SEQ ID NO: 2519; SEQ ID NO: 2520; SEQ ID NO: 2521; SEQ ID NO: 2522; SEQ ID NO: 2523; SEQ ID NO: 2524; SEQ ID NO: 2525; SEQ ID NO: 2526; SEQ ID NO: 2527; SEQ ID NO: 2528; SEQ ID NO: 2529; SEQ ID NO: 2530; SEQ ID NO: 2531; SEQ ID NO: 2532; SEQ ID NO: 2533; SEQ ID NO: 2534; SEQ ID NO: 2535; SEQ ID NO: 2536; SEQ ID NO: 2537; SEQ ID NO: 2538; SEQ ID NO: 2539; SEQ ID NO: 2540; SEQ ID NO: 2541; SEQ ID NO: 2542; SEQ ID NO: 2543; SEQ ID NO: 2544; SEQ ID NO: 2545; SEQ ID NO: 2546; SEQ ID NO: 2547; SEQ ID NO: 2548; SEQ ID NO: 2549; SEQ ID NO: 2550; SEQ ID NO: 2551; SEQ ID NO: 2552; SEQ ID NO: 2553; SEQ ID NO: 2554; SEQ ID NO: 2555; SEQ ID NO: 2556; SEQ ID NO: 2557; SEQ ID NO: 2558; SEQ ID NO: 2559; SEQ ID NO: 2560; SEQ ID NO: 2561; SEQ ID NO: 2562; SEQ ID NO: 2563; SEQ ID NO: 2564; SEQ ID NO: 2565; SEQ ID NO: 2566; SEQ ID NO: 2567; SEQ ID NO: 2568; SEQ ID NO: 2569; SEQ ID NO: 2570; SEQ ID NO: 2571; SEQ ID NO: 2572; SEQ ID NO: 2573; SEQ ID NO: 2574; SEQ ID NO: 2575; SEQ ID NO: 2576; SEQ ID NO: 2577; SEQ ID NO: 2578; SEQ ID NO: 2579; SEQ ID NO: 2580; SEQ ID NO: 2581; SEQ ID NO: 2582; SEQ ID NO: 2583; SEQ ID NO: 2584; SEQ ID NO: 2585; SEQ ID NO: 2586; SEQ ID NO: 2587; SEQ ID NO: 2588; SEQ ID NO: 2589; SEQ ID NO: 2590; SEQ ID NO: 2591; SEQ ID NO: 2592; SEQ ID NO: 2593; SEQ ID NO: 2594; SEQ ID NO: 2595; SEQ ID NO: 2596; SEQ ID NO: 2597; SEQ ID NO: 2598; SEQ ID NO: 2599; SEQ ID NO: 2600; SEQ ID NO: 2601; SEQ ID NO: 2602; SEQ ID NO: 2603; SEQ ID NO: 2604; SEQ ID NO: 2605; SEQ ID NO: 2606; SEQ ID NO: 2607; SEQ ID NO: 2608; SEQ ID NO: 2609; SEQ ID NO: 2610; SEQ ID NO: 2611; SEQ ID NO: 2612; SEQ ID NO: 2613; SEQ ID NO: 2614; SEQ ID NO: 2615; SEQ ID NO: 2616; SEQ ID NO: 2617; SEQ ID NO: 2618; SEQ ID NO: 2619; SEQ ID NO: 2620; SEQ ID NO: 2621; SEQ ID NO: 2622; SEQ ID NO: 2623; SEQ ID NO: 2624; SEQ ID NO: 2625; SEQ ID NO: 2626; SEQ ID NO: 2627; SEQ ID NO: 2628; SEQ ID NO: 2629; SEQ ID NO: 2630; SEQ ID NO: 2631; SEQ ID NO: 2632; SEQ ID NO: 2633; SEQ ID NO: 2634; SEQ ID NO: 2635; SEQ ID NO: 2636; SEQ ID NO: 2637; SEQ ID NO: 2638; SEQ ID NO: 2639; SEQ ID NO: 2640; SEQ ID NO: 2641; SEQ ID NO: 2642; SEQ ID NO: 2643; SEQ ID NO: 2644; SEQ ID NO: 2645; SEQ ID NO: 2646; SEQ ID NO: 2647; SEQ ID NO: 2648; SEQ ID NO: 2649; SEQ ID NO: 2650; SEQ ID NO: 2651; SEQ ID NO: 2652; SEQ ID NO: 2653; SEQ ID NO: 2654; SEQ ID NO: 2655; SEQ ID NO: 2656; SEQ ID NO: 2657; SEQ ID NO: 2658; SEQ ID NO: 2659; SEQ ID NO: 2660; SEQ ID NO: 2661; SEQ ID NO: 2662; SEQ ID NO: 2663; SEQ ID NO: 2664; SEQ ID NO: 2665; SEQ ID NO: 2666; SEQ ID NO: 2667; SEQ ID NO: 2668; SEQ ID NO: 2669; SEQ ID NO: 2670; SEQ ID NO: 2671; SEQ ID NO: 2672; SEQ ID NO: 2673; SEQ ID NO: 2674; SEQ ID NO: 2675; SEQ ID NO: 2676; SEQ ID NO: 2677; SEQ ID NO: 2678; SEQ ID NO: 2679; SEQ ID NO: 2680; SEQ ID NO: 2681; SEQ ID NO: 2682; SEQ ID NO: 2683; SEQ ID NO: 2684; SEQ ID NO: 2685; SEQ ID NO: 2686; SEQ ID NO: 2687; SEQ ID NO: 2688; SEQ ID NO: 2689; SEQ ID NO: 2690; SEQ ID NO: 2691; SEQ ID NO: 2692; SEQ ID NO: 2693; SEQ ID NO: 2694; SEQ ID NO: 2695; SEQ ID NO: 2696; SEQ ID NO: 2697; SEQ ID NO: 2698; SEQ ID NO: 2699; SEQ ID NO: 2700; SEQ ID NO: 2701; SEQ ID NO: 2702; SEQ ID NO: 2703; SEQ ID NO: 2704; SEQ ID NO: 2705; SEQ ID NO: 2706; SEQ ID NO: 2707; SEQ ID NO: 2708; SEQ ID NO: 2709; SEQ ID NO: 2710; SEQ ID NO: 2711; SEQ ID NO: 2712; SEQ ID NO: 2713; SEQ ID NO: 2714; SEQ ID NO: 2715; SEQ ID NO: 2716; SEQ ID NO: 2717; SEQ ID NO: 2718; SEQ ID NO: 2719; SEQ ID NO: 2720; SEQ ID NO: 2721; SEQ ID NO: 2722; SEQ ID NO: 2723; SEQ ID NO: 2724; SEQ ID NO: 2725; SEQ ID NO: 2726; SEQ ID NO: 2727; SEQ ID NO: 2728; SEQ ID NO: 2729; SEQ ID NO: 2730; SEQ ID NO: 2731; SEQ ID NO: 2732; SEQ ID NO: 2733; SEQ ID NO: 2734; SEQ ID NO: 2735; SEQ ID NO: 2736; SEQ ID NO: 2737; SEQ ID NO: 2738; SEQ ID NO: 2739; SEQ ID NO: 2740; SEQ ID NO: 2741; SEQ ID NO: 2742; SEQ ID NO: 2743; SEQ ID NO: 2744; SEQ ID NO: 2745; SEQ ID NO: 2746; SEQ ID NO: 2747; SEQ ID NO: 2748; SEQ ID NO: 2749; SEQ ID NO: 2750; SEQ ID NO: 2751; SEQ ID NO: 2752; SEQ ID NO: 2753; SEQ ID NO: 2754; SEQ ID NO: 2755; SEQ ID NO: 2756; SEQ ID NO: 2757; SEQ ID NO: 2758; SEQ ID NO: 2759; SEQ ID NO: 2760; SEQ ID NO: 2761; SEQ ID NO: 2762; SEQ ID NO: 2763; SEQ ID NO: 2764; SEQ ID NO: 2765; SEQ ID NO: 2766; SEQ ID NO: 2767; SEQ ID NO: 2768; SEQ ID NO: 2769; SEQ ID NO: 2770; SEQ ID NO: 2771; SEQ ID NO: 2772; SEQ ID NO: 2773; SEQ ID NO: 2774; SEQ ID NO: 2775; SEQ ID NO: 2776; SEQ ID NO: 2777; SEQ ID NO: 2778; SEQ ID NO: 2779; SEQ ID NO: 2780; SEQ ID NO: 2781; SEQ ID NO: 2782; SEQ ID NO: 2783; SEQ ID NO: 2784; SEQ ID NO: 2785; SEQ ID NO: 2786; SEQ ID NO: 2787; SEQ ID NO: 2788; SEQ ID NO: 2789; SEQ ID NO: 2790; SEQ ID NO: 2791; SEQ ID NO: 2792; SEQ ID NO: 2793; SEQ ID NO: 2794; SEQ ID NO: 2795; SEQ ID NO: 2796; SEQ ID NO: 2797; SEQ ID NO: 2798; SEQ ID NO: 2799; SEQ ID NO: 2800; SEQ ID NO: 2801; SEQ ID NO: 2802; SEQ ID NO: 2803; SEQ ID NO: 2804; SEQ ID NO: 2805; SEQ ID NO: 2806; SEQ ID NO: 2807; SEQ ID NO: 2808; SEQ ID NO: 2809; SEQ ID NO: 2810; SEQ ID NO: 2811; SEQ ID NO: 2812; SEQ ID NO: 2813; SEQ ID NO: 2814; SEQ ID NO: 2815; SEQ ID NO: 2816; SEQ ID NO: 2817; SEQ ID NO: 2818; SEQ ID NO: 2819; SEQ ID NO: 2820; SEQ ID NO: 2821; SEQ ID NO: 2822; SEQ ID NO: 2823; SEQ ID NO: 2824; SEQ ID NO: 2825; SEQ ID NO: 2826; SEQ ID NO: 2827; SEQ ID NO: 2828; SEQ ID NO: 2829; SEQ ID NO: 2830; SEQ ID NO: 2831; SEQ ID NO: 2832; SEQ ID NO: 2833; SEQ ID NO: 2834; SEQ ID NO: 2835; SEQ ID NO: 2836; SEQ ID NO: 2837; SEQ ID NO: 2838; SEQ ID NO: 2839; SEQ ID NO: 2840; SEQ ID NO: 2841; SEQ ID NO: 2842; SEQ ID NO: 2843; SEQ ID NO: 2844; SEQ ID NO: 2845; SEQ ID NO: 2846; SEQ ID NO: 2847; SEQ ID NO: 2848; SEQ ID NO: 2849; SEQ ID NO: 2850; SEQ ID NO: 2851; SEQ ID NO: 2852; SEQ ID NO: 2853; SEQ ID NO: 2854; SEQ ID NO: 2855; SEQ ID NO: 2856; SEQ ID NO: 2857; SEQ ID NO: 2858; SEQ ID NO: 2859; SEQ ID NO: 2860; SEQ ID NO: 2861; SEQ ID NO: 2862; SEQ ID NO: 2863; SEQ ID NO: 2864; SEQ ID NO: 2865; SEQ ID NO: 2866; SEQ ID NO: 2867; SEQ ID NO: 2868; SEQ ID NO: 2869; SEQ ID NO: 2870; SEQ ID NO: 2871; SEQ ID NO: 2872; SEQ ID NO: 2873; SEQ ID NO: 2874; SEQ ID NO: 2875; SEQ ID NO: 2876; SEQ ID NO: 2877; SEQ ID NO: 2878; SEQ ID NO: 2879; SEQ ID NO: 2880; SEQ ID NO: 2881; SEQ ID NO: 2882; SEQ ID NO: 2883; SEQ ID NO: 2884; SEQ ID NO: 2885; SEQ ID NO: 2886; SEQ ID NO: 2887; SEQ ID NO: 2888; SEQ ID NO: 2889; SEQ ID NO: 2890; SEQ ID NO: 2891; SEQ ID NO: 2892; SEQ ID NO: 2893; SEQ ID NO: 2894; SEQ ID NO: 2895; SEQ ID NO: 2896; SEQ ID NO: 2897; SEQ ID NO: 2898; SEQ ID NO: 2899; SEQ ID NO: 2900; SEQ ID NO: 2901; SEQ ID NO: 2902; SEQ ID NO: 2903; SEQ ID NO: 2904; SEQ ID NO: 2905; SEQ ID NO: 2906; SEQ ID NO: 2907; SEQ ID NO: 2908; SEQ ID NO: 2909; SEQ ID NO: 2910; SEQ ID NO: 2911; SEQ ID NO: 2912; SEQ ID NO: 2913; SEQ ID NO: 2914; SEQ ID NO: 2915; SEQ ID NO: 2916; SEQ ID NO: 2917; SEQ ID NO: 2918; SEQ ID NO: 2919; SEQ ID NO: 2920; SEQ ID NO: 2921; SEQ ID NO: 2922; SEQ ID NO: 2923; SEQ ID NO: 2924; SEQ ID NO: 2925; SEQ ID NO: 2926; SEQ ID NO: 2927; SEQ ID NO: 2928; SEQ ID NO: 2929; SEQ ID NO: 2930; SEQ ID NO: 2931; SEQ ID NO: 2932; SEQ ID NO: 2933; SEQ ID NO: 2934; SEQ ID NO: 2935; SEQ ID NO: 2936; SEQ ID NO: 2937; SEQ ID NO: 2938; SEQ ID NO: 2939; SEQ ID NO: 2940; SEQ ID NO: 2941; SEQ ID NO: 2942; SEQ ID NO: 2943; SEQ ID NO: 2944; SEQ ID NO: 2945; SEQ ID NO: 2946; SEQ ID NO: 2947; SEQ ID NO: 2948; SEQ ID NO: 2949; SEQ ID NO: 2950; SEQ ID NO: 2951; SEQ ID NO: 2952; SEQ ID NO: 2953; SEQ ID NO: 2954; SEQ ID NO: 2955; SEQ ID NO: 2956; SEQ ID NO: 2957; SEQ ID NO: 2958; SEQ ID NO: 2959; SEQ ID NO: 2960; SEQ ID NO: 2961; SEQ ID NO: 2962; SEQ ID NO: 2963; SEQ ID NO: 2964; SEQ ID NO: 2965; SEQ ID NO: 2966; SEQ ID NO: 2967; SEQ ID NO: 2968; SEQ ID NO: 2969; SEQ ID NO: 2970; SEQ ID NO: 2971; SEQ ID NO: 2972; SEQ ID NO: 2973; SEQ ID NO: 2974; SEQ ID NO: 2975; SEQ ID NO: 2976; SEQ ID NO: 2977; SEQ ID NO: 2978; SEQ ID NO: 2979; SEQ ID NO: 2980; SEQ ID NO: 2981; SEQ ID NO: 2982; SEQ ID NO: 2983; SEQ ID NO: 2984; SEQ ID NO: 2985; SEQ ID NO: 2986; SEQ ID NO: 2987; SEQ ID NO: 2988; SEQ ID NO: 2989; SEQ ID NO: 2990; SEQ ID NO: 2991; SEQ ID NO: 2992; SEQ ID NO: 2993; SEQ ID NO: 2994; SEQ ID NO: 2995; SEQ ID NO: 2996; SEQ ID NO: 2997; SEQ ID NO: 2998; SEQ ID NO: 2999; SEQ ID NO: 3000; SEQ ID NO: 3001; SEQ ID NO: 3002; SEQ ID NO: 3003; SEQ ID NO: 3004; SEQ ID NO: 3005; SEQ ID NO: 3006; SEQ ID NO: 3007; SEQ ID NO: 3008; SEQ ID NO: 3009; SEQ ID NO: 3010; SEQ ID NO: 3011; SEQ ID NO: 3012; SEQ ID NO: 3013; SEQ ID NO: 3014; SEQ ID NO: 3015; SEQ ID NO: 3016; SEQ ID NO: 3017; SEQ ID NO: 3018; SEQ ID NO: 3019; SEQ ID NO: 3020; SEQ ID NO: 3021; SEQ ID NO: 3022; SEQ ID NO: 3023; SEQ ID NO: 3024; SEQ ID NO: 3025; SEQ ID NO: 3026; SEQ ID NO: 3027; SEQ ID NO: 3028; SEQ ID NO: 3029; SEQ ID NO: 3030; SEQ ID NO: 3031; SEQ ID NO: 3032; SEQ ID NO: 3033; SEQ ID NO: 3034; SEQ ID NO: 3035; SEQ ID NO: 3036; SEQ ID NO: 3037; SEQ ID NO: 3038; SEQ ID NO: 3039; SEQ ID NO: 3040; SEQ ID NO: 3041; SEQ ID NO: 3042; SEQ ID NO: 3043; SEQ ID NO: 3044; SEQ ID NO: 3045; SEQ ID NO: 3046; SEQ ID NO: 3047; SEQ ID NO: 3048; SEQ ID NO: 3049; SEQ ID NO: 3050; SEQ ID NO: 3051; SEQ ID NO: 3052; SEQ ID NO: 3053; SEQ ID NO: 3054; SEQ ID NO: 3055; SEQ ID NO: 3056; SEQ ID NO: 3057; SEQ ID NO: 3058; SEQ ID NO: 3059; SEQ ID NO: 3060; SEQ ID NO: 3061; SEQ ID NO: 3062; SEQ ID NO: 3063; SEQ ID NO: 3064; SEQ ID NO: 3065; SEQ ID NO: 3066; SEQ ID NO: 3067; SEQ ID NO: 3068; SEQ ID NO: 3069; SEQ ID NO: 3070; SEQ ID NO: 3071; SEQ ID NO: 3072; SEQ ID NO: 3073; SEQ ID NO: 3074; SEQ ID NO: 3075; SEQ ID NO: 3076; SEQ ID NO: 3077; SEQ ID NO: 3078; SEQ ID NO: 3079; SEQ ID NO: 3080; SEQ ID NO: 3081; SEQ ID NO: 3082; SEQ ID NO: 3083; SEQ ID NO: 3084; SEQ ID NO: 3085; SEQ ID NO: 3086; SEQ ID NO: 3087; SEQ ID NO: 3088; SEQ ID NO: 3089; SEQ ID NO: 3090; SEQ ID NO: 3091; SEQ ID NO: 3092; SEQ ID NO: 3093; SEQ ID NO: 3094; SEQ ID NO: 3095; SEQ ID NO: 3096; SEQ ID NO: 3097; SEQ ID NO: 3098; SEQ ID NO: 3099; SEQ ID NO: 3100; SEQ ID NO: 3101; SEQ ID NO: 3102; SEQ ID NO: 3103; SEQ ID NO: 3104; SEQ ID NO: 3105; SEQ ID NO: 3106; SEQ ID NO: 3107; SEQ ID NO: 3108; SEQ ID NO: 3109; SEQ ID NO: 3110; SEQ ID NO: 3111; SEQ ID NO: 3112; SEQ ID NO: 3113; SEQ ID NO: 3114; SEQ ID NO: 3115; SEQ ID NO: 3116; SEQ ID NO: 3117; SEQ ID NO: 3118; SEQ ID NO: 3119; SEQ ID NO: 3120; SEQ ID NO: 3121; SEQ ID NO: 3122; SEQ ID NO: 3123; SEQ ID NO: 3124; SEQ ID NO: 3125; SEQ ID NO: 3126; SEQ ID NO: 3127; SEQ ID NO: 3128; SEQ ID NO: 3129; SEQ ID NO: 3130; SEQ ID NO: 3131; SEQ ID NO: 3132; SEQ ID NO: 3133; SEQ ID NO: 3134; SEQ ID NO: 3135; SEQ ID NO: 3136; SEQ ID NO: 3137; SEQ ID NO: 3138; SEQ ID NO: 3139; SEQ ID NO: 3140; SEQ ID NO: 3141; SEQ ID NO: 3142; SEQ ID NO: 3143; SEQ ID NO: 3144; SEQ ID NO: 3145; SEQ ID NO: 3146; SEQ ID NO: 3147; SEQ ID NO: 3148; SEQ ID NO: 3149; SEQ ID NO: 3150; SEQ ID NO: 3151; SEQ ID NO: 3152; SEQ ID NO: 3153; SEQ ID NO: 3154; SEQ ID NO: 3155; SEQ ID NO: 3156; SEQ ID NO: 3157; SEQ ID NO: 3158; SEQ ID NO: 3159; SEQ ID NO: 3160; SEQ ID NO: 3161; SEQ ID NO: 3162; SEQ ID NO: 3163; SEQ ID NO: 3164; SEQ ID NO: 3165; SEQ ID NO: 3166; SEQ ID NO: 3167; SEQ ID NO: 3168; SEQ ID NO: 3169; SEQ ID NO: 3170; SEQ ID NO: 3171; SEQ ID NO: 3172; SEQ ID NO: 3173; SEQ ID NO: 3174; SEQ ID NO: 3175; SEQ ID NO: 3176; SEQ ID NO: 3177; SEQ ID NO: 3178; SEQ ID NO: 3179; SEQ ID NO: 3180; SEQ ID NO: 3181; SEQ ID NO: 3182; SEQ ID NO: 3183; SEQ ID NO: 3184; SEQ ID NO: 3185; SEQ ID NO: 3186; SEQ ID NO: 3187; SEQ ID NO: 3188; SEQ ID NO: 3189; SEQ ID NO: 3190; SEQ ID NO: 3191; SEQ ID NO: 3192; SEQ ID NO: 3193; SEQ ID NO: 3194; SEQ ID NO: 3195; SEQ ID NO: 3196; SEQ ID NO: 3197; SEQ ID NO: 3198; SEQ ID NO: 3199; SEQ ID NO: 3200; SEQ ID NO: 3201; SEQ ID NO: 3202; SEQ ID NO: 3203; SEQ ID NO: 3204; SEQ ID NO: 3205; SEQ ID NO: 3206; SEQ ID NO: 3207; SEQ ID NO: 3208; SEQ ID NO: 3209; SEQ ID NO: 3210; SEQ ID NO: 3211; SEQ ID NO: 3212; SEQ ID NO: 3213; SEQ ID NO: 3214; SEQ ID NO: 3215; SEQ ID NO: 3216; SEQ ID NO: 3217; SEQ ID NO: 3218; SEQ ID NO: 3219; SEQ ID NO: 3220; SEQ ID NO: 3221; SEQ ID NO: 3222; SEQ ID NO: 3223; SEQ ID NO: 3224; SEQ ID NO: 3225; SEQ ID NO: 3226; SEQ ID NO: 3227; SEQ ID NO: 3228; SEQ ID NO: 3229; SEQ ID NO: 3230; SEQ ID NO: 3231; SEQ ID NO: 3232; SEQ ID NO: 3233; SEQ ID NO: 3234; SEQ ID NO: 3235; SEQ ID NO: 3236; SEQ ID NO: 3237; SEQ ID NO: 3238; SEQ ID NO: 3239; SEQ ID NO: 3240; SEQ ID NO: 3241; SEQ ID NO: 3242; SEQ ID NO: 3243; SEQ ID NO: 3244; SEQ ID NO: 3245; SEQ ID NO: 3246; SEQ ID NO: 3247; SEQ ID NO: 3248; SEQ ID NO: 3249; SEQ ID NO: 3250; SEQ ID NO: 3251; SEQ ID NO: 3252; SEQ ID NO: 3253; SEQ ID NO: 3254; SEQ ID NO: 3255; SEQ ID NO: 3256; SEQ ID NO: 3257; SEQ ID NO: 3258; SEQ ID NO: 3259; SEQ ID NO: 3260; SEQ ID NO: 3261; SEQ ID NO: 3262; SEQ ID NO: 3263; SEQ ID NO: 3264; SEQ ID NO: 3265; SEQ ID NO: 3266; SEQ ID NO: 3267; SEQ ID NO: 3268; SEQ ID NO: 3269; SEQ ID NO: 3270; SEQ ID NO: 3271; SEQ ID NO: 3272; SEQ ID NO: 3273; SEQ ID NO: 3274; SEQ ID NO: 3275; SEQ ID NO: 3276; SEQ ID NO: 3277; SEQ ID NO: 3278; SEQ ID NO: 3279; SEQ ID NO: 3280; SEQ ID NO: 3281; SEQ ID NO: 3282; SEQ ID NO: 3283; SEQ ID NO: 3284; SEQ ID NO: 3285; SEQ ID NO: 3266, SEQ ID NO: 3287; SEQ ID NO: 3288; SEQ ID NO: 3289; SEQ ID NO: 3290; SEQ ID NO: 3291; SEQ ID NO: 3292; SEQ ID NO: 3293; SEQ ID NO: 3294; SEQ ID NO: 3295; SEQ ID NO: 3296; SEQ ID NO: 3297; SEQ ID NO: 3298; SEQ ID NO: 3299; SEQ ID NO: 3300; SEQ ID NO: 3301; SEQ ID NO: 3302; SEQ ID NO: 3303; SEQ ID NO: 3304; SEQ ID NO: 3305; SEQ ID NO: 3306; SEQ ID NO: 3307; SEQ ID NO: 3308; SEQ ID NO: 3309; SEQ ID NO: 3310; SEQ ID NO: 3311; SEQ ID NO: 3312; SEQ ID NO: 3313; SEQ ID NO: 3314; SEQ ID NO: 3315; SEQ ID NO: 3316; SEQ ID NO: 3317; SEQ ID NO:3318; SEQ ID NO: 3319; SEQ ID NO: 3320; SEQ ID NO: 3321; SEQ ID NO: 3322; SEQ ID NO: 3323; SEQ ID NO: 3324; SEQ ID NO: 3325; SEQ ID NO: 3326; SEQ ID NO: 3327; SEQ ID NO: 3328; SEQ ID NO: 3329; SEQ ID NO: 3330; SEQ ID NO: 3331; SEQ ID NO: 3332; SEQ ID NO: 3333; SEQ ID NO: 3334; SEQ ID NO: 3335; SEQ ID NO: 3336; SEQ ID NO: 3337; SEQ ID NO: 3338; SEQ ID NO: 3339; SEQ ID NO: 3340; SEQ ID NO: 3341; SEQ ID NO: 3342; SEQ ID NO: 3343; SEQ ID NO: 3344; SEQ ID NO: 3345; SEQ ID NO: 3346; SEQ ID NO: 3347; SEQ ID NO: 3348; SEQ ID NO: 3349; SEQ ID NO: 3350; SEQ ID NO: 3351; SEQ ID NO: 3352; SEQ ID NO: 3353; SEQ ID NO: 3354; SEQ ID NO: 3355; SEQ ID NO: 3356; SEQ ID NO: 3357; SEQ ID NO: 3358; SEQ ID NO: 3359; SEQ ID NO: 3360; SEQ ID NO: 3361; SEQ ID NO: 3362; SEQ ID NO: 3363; SEQ ID NO: 3364; SEQ ID NO: 3365; SEQ ID NO: 3366; SEQ ID NO: 3367; SEQ ID NO: 3368; SEQ ID NO: 3369; SEQ ID NO: 3370; SEQ ID NO: 3371; SEQ ID NO: 3372; SEQ ID NO: 3373; SEQ ID NO: 3374; SEQ ID NO: 3375; SEQ ID NO: 3376; SEQ ID NO: 3377; SEQ ID NO: 3378; SEQ ID NO: 3379; SEQ ID NO: 3380; SEQ ID NO: 3381; SEQ ID NO: 3382; SEQ ID NO: 3383; SEQ ID NO: 3384; SEQ ID NO: 3385; SEQ ID NO: 3386; SEQ ID NO: 3387; SEQ ID NO: 3388; SEQ ID NO: 3389; SEQ ID NO: 3390; SEQ ID NO: 3391; SEQ ID NO: 3392; SEQ ID NO: 3393; SEQ ID NO: 3394; SEQ ID NO: 3395; SEQ ID NO: 3396; SEQ ID NO: 3397; SEQ ID NO: 3398; SEQ ID NO: 3399; SEQ ID NO: 3400; SEQ ID NO: 3401; SEQ ID NO: 3402; SEQ ID NO: 3403; SEQ ID NO: 3404; SEQ ID NO: 3405; SEQ ID NO: 3406; SEQ ID NO: 3407; SEQ ID NO: 3408; SEQ ID NO: 3409; SEQ ID NO: 3410; SEQ ID NO: 3411; SEQ ID NO: 3412; SEQ ID NO: 3413; SEQ ID NO: 3414; SEQ ID NO: 3415; SEQ ID NO: 3416; SEQ ID NO: 3417; SEQ ID NO: 3418; SEQ ID NO: 3419; SEQ ID NO: 3420; SEQ ID NO: 3421; SEQ ID NO: 3422; SEQ ID NO: 3423; SEQ ID NO: 3424; SEQ ID NO: 3425; SEQ ID NO: 3426; SEQ ID NO: 3427; SEQ ID NO: 3428; SEQ ID NO: 3429; SEQ ID NO: 3430; SEQ ID NO: 3431; SEQ ID NO: 3432; SEQ ID NO: 3433; SEQ ID NO: 3434; SEQ ID NO: 3435; SEQ ID NO: 3436; SEQ ID NO: 3437; SEQ ID NO: 3438; SEQ ID NO: 3439; SEQ ID NO: 3440; SEQ ID NO: 3441; SEQ ID NO: 3442; SEQ ID NO: 3443; SEQ ID NO: 3444; SEQ ID NO: 3445; SEQ ID NO: 3446; SEQ ID NO: 3447; SEQ ID NO: 3448; SEQ ID NO: 3449; SEQ ID NO: 3450; SEQ ID NO: 3451; SEQ ID NO: 3452; SEQ ID NO: 3453; SEQ ID NO: 3454; SEQ ID NO: 3455; SEQ ID NO: 3456; SEQ ID NO: 3457; SEQ ID NO: 3458; SEQ ID NO: 3459; SEQ ID NO: 3460; SEQ ID NO: 3461; SEQ ID NO: 3462; SEQ ID NO: 3463; SEQ ID NO: 3464; SEQ ID NO: 3465; SEQ ID NO: 3466; SEQ ID NO: 3467; SEQ ID NO: 3468; SEQ ID NO: 3469; SEQ ID NO: 3470; SEQ ID NO: 3471; SEQ ID NO: 3472; SEQ ID NO: 3473; SEQ ID NO: 3474; SEQ ID NO: 3475; SEQ ID NO: 3476; SEQ ID NO: 3477; SEQ ID NO: 3478; SEQ ID NO: 3479; SEQ ID NO: 3480; SEQ ID NO: 3481; SEQ ID NO: 3482; SEQ ID NO: 3483; SEQ ID NO: 3484; SEQ ID NO: 3485; SEQ ID NO: 3486; SEQ ID NO: 3487; SEQ ID NO: 3488; SEQ ID NO: 3489; SEQ ID NO: 3490; SEQ ID NO: 3491; SEQ ID NO: 3492; SEQ ID NO: 3493; SEQ ID NO: 3494; SEQ ID NO: 3495; SEQ ID NO: 3496; SEQ ID NO: 3497; SEQ ID NO: 3498; SEQ ID NO: 3499; SEQ ID NO: 3500; SEQ ID NO: 3501; SEQ ID NO: 3502; SEQ ID NO: 3503; SEQ ID NO: 3504; SEQ ID NO: 3505; SEQ ID NO: 3506; SEQ ID NO: 3507; SEQ ID NO: 3508; SEQ ID NO: 3509; SEQ ID NO: 3510; SEQ ID NO: 3511; SEQ ID NO: 3512; SEQ ID NO: 3513; SEQ ID NO: 3514; SEQ ID NO: 3515; SEQ ID NO: 3516; SEQ ID NO: 3517; SEQ ID NO: 3518; SEQ ID NO: 3519; SEQ ID NO: 3520; SEQ ID NO: 3521; SEQ ID NO: 3522; SEQ ID NO: 3523; SEQ ID NO: 3524; SEQ ID NO: 3525; SEQ ID NO: 3526; SEQ ID NO: 3527; SEQ ID NO: 3528; SEQ ID NO: 3529; SEQ ID NO: 3530; SEQ ID NO: 3531; SEQ ID NO: 3532; SEQ ID NO: 3533; SEQ ID NO: 3534; SEQ ID NO: 3535; SEQ ID NO: 3536; SEQ ID NO: 3537; SEQ ID NO: 3538; SEQ ID NO: 3539; SEQ ID NO: 3540; SEQ ID NO: 3541; SEQ ID NO: 3542; SEQ ID NO: 3543; SEQ ID NO: 3544; SEQ ID NO: 3545; SEQ ID NO: 3546; SEQ ID NO: 3547; SEQ ID NO: 3548; SEQ ID NO: 3549; SEQ ID NO: 3550; SEQ ID NO: 3551; SEQ ID NO: 3552; SEQ ID NO: 3553; SEQ ID NO: 3554; SEQ ID NO: 3555; SEQ ID NO: 3556; SEQ ID NO: 3557; SEQ ID NO: 3558; SEQ ID NO: 3559; SEQ ID NO: 3560; SEQ ID NO: 3561; SEQ ID NO: 3562; SEQ ID NO: 3563; SEQ ID NO: 3564; SEQ ID NO: 3565; SEQ ID NO: 3566; SEQ ID NO: 3567; SEQ ID NO: 3568; SEQ ID NO: 3569; SEQ ID NO: 3570; SEQ ID NO: 3571; SEQ ID NO: 3572; SEQ ID NO: 3573; SEQ ID NO: 3574; SEQ ID NO: 3575; SEQ ID NO: 3576; SEQ ID NO: 3577; SEQ ID NO: 3578; SEQ ID NO: 3579; SEQ ID NO: 3580; SEQ ID NO: 3581; SEQ ID NO: 3582; SEQ ID NO: 3583; SEQ ID NO: 3584; SEQ ID NO: 3585; SEQ ID NO: 3586; SEQ ID NO: 3587; SEQ ID NO: 3588; SEQ ID NO: 3589; SEQ ID NO: 3590; SEQ ID NO: 3591; SEQ ID NO: 3592; SEQ ID NO: 3593; SEQ ID NO: 3594; SEQ ID NO: 3595; SEQ ID NO: 3596; SEQ ID NO: 3597; SEQ ID NO: 3598; SEQ ID NO: 3599; SEQ ID NO: 3600; SEQ ID NO: 3601; SEQ ID NO: 3602; SEQ ID NO: 3603; SEQ ID NO: 3604; SEQ ID NO: 3605; SEQ ID NO: 3606; SEQ ID NO: 3607; SEQ ID NO: 3608; SEQ ID NO: 3609; SEQ ID NO: 3610; SEQ ID NO: 3611; SEQ ID NO: 3612; SEQ ID NO: 3613; SEQ ID NO 3614; SEQ ID NO: 3615; SEQ ID NO: 3616; SEQ ID NO: 3617; SEQ ID NO: 3618; SEQ ID NO: 3619; SEQ ID NO: 3620; SEQ ID NO: 3621; SEQ ID NO: 3622; SEQ ID NO: 3623; SEQ ID NO: 3624; SEQ ID NO: 3625; SEQ ID NO: 3626; SEQ ID NO: 3627; SEQ ID NO: 3628; SEQ ID NO: 3629; SEQ ID NO: 3630; SEQ ID NO: 3631; SEQ ID NO: 3632; SEQ ID NO: 3633; SEQ ID NO: 3634; SEQ ID NO: 3635; SEQ ID NO: 3636; SEQ ID NO: 3637; SEQ ID NO: 3638; SEQ ID NO: 3639; SEQ ID NO: 3640; SEQ ID NO: 3641; SEQ ID NO: 3642; SEQ ID NO: 3643; SEQ ID NO: 3644; SEQ ID NO: 3645; SEQ ID NO: 3646; SEQ ID NO: 3647; SEQ ID NO: 3648; SEQ ID NO: 3649; SEQ ID NO: 3650; SEQ ID NO: 3651; SEQ ID NO: 3652; SEQ ID NO: 3653; SEQ ID NO: 3654; SEQ ID NO: 3655; SEQ ID NO: 3656; SEQ ID NO: 3657; SEQ ID NO: 3658; SEQ ID NO: 3659; SEQ ID NO: 3660; SEQ ID NO: 3661; SEQ ID NO: 3662; SEQ ID NO: 3663; SEQ ID NO: 3664; SEQ ID NO: 3665; SEQ ID NO: 3666; SEQ ID NO: 3667; SEQ ID NO: 3668; SEQ ID NO: 3669; SEQ ID NO: 3670; SEQ ID NO: 3671; SEQ ID NO: 3672; SEQ ID NO: 3673; SEQ ID NO: 3674; SEQ ID NO: 3675; SEQ ID NO: 3676; SEQ ID NO: 3677; SEQ ID NO: 3678; SEQ ID NO: 3679; SEQ ID NO: 3680; SEQ ID NO: 3681; SEQ ID NO: 3682; SEQ ID NO: 3683; SEQ ID NO: 3684; SEQ ID NO: 3685; SEQ ID NO: 3686; SEQ ID NO: 3687; SEQ ID NO: 3688; SEQ ID NO: 3689; SEQ ID NO: 3690; SEQ ID NO: 3691; SEQ ID NO: 3692; SEQ ID NO: 3693; SEQ ID NO: 3694; SEQ ID NO: 3695; SEQ ID NO: 3696; SEQ ID NO: 3697; SEQ ID NO: 3698; SEQ ID NO: 3699; SEQ ID NO: 3700; SEQ ID NO: 3701; SEQ ID NO: 3702; SEQ ID NO: 3703; SEQ ID NO: 3704; SEQ ID NO: 3705; SEQ ID NO: 3706; SEQ ID NO: 3707; SEQ ID NO: 3708; SEQ ID NO: 3709; SEQ ID NO: 3710; SEQ ID NO: 3711; SEQ ID NO: 3712; SEQ ID NO: 3713; SEQ ID NO: 3714; SEQ ID NO: 3715; SEQ ID NO: 3716; SEQ ID NO: 3717; SEQ ID NO: 3718; SEQ ID NO: 3719; SEQ ID NO: 3720; SEQ ID NO: 3721; SEQ ID NO: 3722; SEQ ID NO: 3723; SEQ ID NO: 3724; SEQ ID NO: 3725; SEQ ID NO: 3726; SEQ ID NO: 3727; SEQ ID NO: 3728; SEQ ID NO: 3729; SEQ ID NO: 3730; SEQ ID NO: 3731; SEQ ID NO: 3732; SEQ ID NO: 3733; SEQ ID NO: 3734; SEQ ID NO: 3735; SEQ ID NO: 3736; SEQ ID NO: 3737; SEQ ID NO: 3738; SEQ ID NO: 3739; SEQ ID NO: 3740; SEQ ID NO: 3741; SEQ ID NO: 3742; SEQ ID NO: 3743; SEQ ID NO: 3744; SEQ ID NO: 3745; SEQ ID NO: 3746; SEQ ID NO: 3747; SEQ ID NO: 3748; SEQ ID NO: 3749; SEQ ID NO: 3750; SEQ ID NO: 3751; SEQ ID NO: 3752; SEQ ID NO: 3753; SEQ ID NO: 3754; SEQ ID NO: 3755; SEQ ID NO: 3756; SEQ ID NO: 3757; SEQ ID NO: 3758; SEQ ID NO: 3759; SEQ ID NO: 3760; SEQ ID NO: 3761; SEQ ID NO: 3762; SEQ ID NO: 3763; SEQ ID NO: 3764; SEQ ID NO: 3765; SEQ ID NO: 3766; SEQ ID NO: 3767; SEQ ID NO: 3768; SEQ ID NO: 3769; SEQ ID NO: 3770; SEQ ID NO: 3771; SEQ ID NO: 3772; SEQ ID NO: 3773; SEQ ID NO: 3774; SEQ ID NO: 3775; SEQ ID NO: 3776; SEQ ID NO: 3777; SEQ ID NO: 3778; SEQ ID NO: 3779; SEQ ID NO: 3780; SEQ ID NO: 3781; SEQ ID NO: 3782; SEQ ID NO: 3783; SEQ ID NO: 3784; SEQ ID NO: 3785; SEQ ID NO: 3786; SEQ ID NO: 3787; SEQ ID NO: 3788; SEQ ID NO: 3789; SEQ ID NO: 3790; SEQ ID NO: 3791; SEQ ID NO: 3792; SEQ ID NO: 3793; SEQ ID NO: 3794; SEQ ID NO: 3795; SEQ ID NO: 3796; SEQ ID NO: 3797; SEQ ID NO: 3798; SEQ ID NO: 3799; SEQ ID NO: 3800; SEQ ID NO: 3801; SEQ ID NO: 3802; SEQ ID NO: 3803; SEQ ID NO: 3804; SEQ ID NO: 3805; SEQ ID NO: 3806; SEQ ID NO: 3807; SEQ ID NO: 3808; SEQ ID NO: 3809; SEQ ID NO: 3810; SEQ ID NO: 3811; SEQ ID NO: 3812; SEQ ID NO: 3813; SEQ ID NO: 3814; SEQ ID NO: 3815; SEQ ID NO: 3816; SEQ ID NO: 3817; SEQ ID NO: 3818; SEQ ID NO: 3819; SEQ ID NO: 3820; SEQ ID NO: 3821; SEQ ID NO: 3822; SEQ ID NO: 3823; SEQ ID NO: 3824; SEQ ID NO: 3825; SEQ ID NO: 3826; SEQ ID NO: 3827; SEQ ID NO: 3828; SEQ ID NO: 3829; SEQ ID NO: 3830; SEQ ID NO: 3831; SEQ ID NO: 3832; SEQ ID NO: 3833; SEQ ID NO: 3834; SEQ ID NO: 3835; SEQ ID NO: 3836; SEQ ID NO: 3837; SEQ ID NO: 3838; SEQ ID NO: 3839; SEQ ID NO: 3840; SEQ ID NO: 3841; SEQ ID NO: 3842; SEQ ID NO: 3843; SEQ ID NO: 3844; SEQ ID NO: 3845; SEQ ID NO: 3846; SEQ ID NO: 3847; SEQ ID NO: 3848; SEQ ID NO: 3849; SEQ ID NO: 3850; SEQ ID NO: 3851; SEQ ID NO: 3852; SEQ ID NO: 3853; SEQ ID NO: 3854; SEQ ID NO: 3855; SEQ ID NO: 3856; SEQ ID NO: 3857; SEQ ID NO: 3858; SEQ ID NO: 3859; SEQ ID NO: 3860; SEQ ID NO: 3861; SEQ ID NO: 3862; SEQ ID NO: 3863; SEQ ID NO: 3864; SEQ ID NO: 3865; SEQ ID NO: 3866; SEQ ID NO: 3867; SEQ ID NO: 3868; SEQ ID NO: 3869; SEQ ID NO: 3870; SEQ ID NO: 3871; SEQ ID NO: 3872; SEQ ID NO: 3873; SEQ ID NO: 3874; SEQ ID NO: 3875; SEQ ID NO: 3876; SEQ ID NO: 3877; SEQ ID NO: 3878; SEQ ID NO: 3879; SEQ ID NO: 3880; SEQ ID NO: 3881; SEQ ID NO: 3882; SEQ ID NO: 3883; SEQ ID NO: 3884; SEQ ID NO: 3885; SEQ ID NO: 3886; SEQ ID NO: 3887; SEQ ID NO: 3888; SEQ ID NO: 3889; SEQ ID NO: 3890; SEQ ID NO: 3891; SEQ ID NO: 3892; SEQ ID NO: 3893; SEQ ID NO: 3894; SEQ ID NO: 3895; SEQ ID NO: 3896; SEQ ID NO: 3897; SEQ ID NO: 3898; SEQ ID NO: 3899; SEQ ID NO: 3900; SEQ ID NO: 3901; SEQ ID NO: 3902; SEQ ID NO: 3903; SEQ ID NO: 3904; SEQ ID NO: 3905; SEQ ID NO: 3906; SEQ ID NO: 3907; SEQ ID NO: 3908; SEQ ID NO: 3909; SEQ ID NO: 3910; SEQ ID NO: 3911; SEQ ID NO: 3912; SEQ ID NO: 3913; SEQ ID NO: 3914; SEQ ID NO: 3915; SEQ ID NO: 3916; SEQ ID NO: 3917; SEQ ID NO: 3918; SEQ ID NO: 3919; SEQ ID NO: 3920; SEQ ID NO: 3921; SEQ ID NO: 3922; SEQ ID NO: 3923; SEQ ID NO: 3924; SEQ ID NO: 3925; SEQ ID NO: 3926; SEQ ID NO: 3927; SEQ ID NO: 3928; SEQ ID NO: 3929; SEQ ID NO: 3930; SEQ ID NO: 3931; SEQ ID NO: 3932; SEQ ID NO: 3933; SEQ ID NO: 3934; SEQ ID NO: 3935; SEQ ID NO: 3936; SEQ ID NO: 3937; SEQ ID NO: 3938; SEQ ID NO: 3939; SEQ ID NO: 3940; SEQ ID NO: 3941; SEQ ID NO: 3942; SEQ ID NO: 3943; SEQ ID NO: 3944; SEQ ID NO: 3945; SEQ ID NO: 3946; SEQ ID NO: 3947; SEQ ID NO: 3948; SEQ ID NO: 3949; SEQ ID NO: 3950; SEQ ID NO: 3951; SEQ ID NO: 3952; SEQ ID NO: 3953; SEQ ID NO: 3954; SEQ ID NO: 3955; SEQ ID NO: 3956; SEQ ID NO: 3957; SEQ ID NO: 3958; SEQ ID NO: 3959; SEQ ID NO: 3960; SEQ ID NO: 3961; SEQ ID NO: 3962; SEQ ID NO: 3963; SEQ ID NO: 3964; SEQ ID NO: 3965; SEQ ID NO: 3966; SEQ ID NO: 3967; SEQ ID NO: 3968; SEQ ID NO: 3969; SEQ ID NO: 3970; SEQ ID NO: 3971; SEQ ID NO: 3972; SEQ ID NO: 3973; SEQ ID NO: 3974; SEQ ID NO: 3975; SEQ ID NO: 3976; SEQ ID NO: 3977; SEQ ID NO: 3978; SEQ ID NO: 3979; SEQ ID NO: 3980; SEQ ID NO: 3981; SEQ ID NO: 3982; SEQ ID NO: 3983; SEQ ID NO: 3984; SEQ ID NO: 3985; SEQ ID NO: 3986; SEQ ID NO: 3987; SEQ ID NO: 3988; SEQ ID NO: 3989; SEQ ID NO: 3990; SEQ ID NO: 3991; SEQ ID NO: 3992; SEQ ID NO: 3993; SEQ ID NO: 3994; SEQ ID NO: 3995; SEQ ID NO: 3996; SEQ ID NO: 3997; SEQ ID NO: 3998; SEQ ID NO: 3999; SEQ ID NO: 4000; SEQ ID NO: 4001; SEQ ID NO: 4002; SEQ ID NO: 4003; SEQ ID NO: 4004; SEQ ID NO: 4005; SEQ ID NO: 4006; SEQ ID NO: 4007; SEQ ID NO: 4008; SEQ ID NO: 4009; SEQ ID NO: 4010; SEQ ID NO: 4011; SEQ ID NO: 4012; SEQ ID NO: 4013; SEQ ID NO: 4014; SEQ ID NO: 4015; SEQ ID NO: 9593; SEQ ID NO: 9594; SEQ ID NO: 9595; SEQ ID NO: 9596; SEQ ID NO: 9597; SEQ ID NO: 9598; SEQ ID NO: 9599; SEQ ID NO: 9600; SEQ ID NO: 9601; SEQ ID NO: 9602; SEQ ID NO: 9603; SEQ ID NO: 9604; SEQ ID NO: 9605; SEQ ID NO: 9606; SEQ ID NO: 9607; SEQ ID NO: 9608; SEQ ID NO: 9609; SEQ ID NO: 9610; SEQ ID NO: 9611; SEQ ID NO: 9612; SEQ ID NO: 9613; SEQ ID NO: 9614; SEQ ID NO: 9615; SEQ ID NO: 9616; SEQ ID NO: 9617; SEQ ID NO: 9618; SEQ ID NO: 9619; SEQ ID NO: 9620 and SEQ ID NO: 9621.

In one embodiment, the H. pylori cytoplasmic polypeptide or a fragment thereof is an H. pylori polypeptide involved in energy metabolism or a fragment thereof encoded by a nucleic acid having a nucleotide sequence selected from the group consisting of SEQ ID NO: 1576; SEQ ID NO: 1576; SEQ ID NO: 1577; SEQ ID NO: 1578; SEQ ID NO: 1579; SEQ ID NO: 1580; SEQ ID NO: 1581; SEQ ID NO: 1582; SEQ ID NO: 1583; SEQ ID NO: 1584; SEQ ID NO: 1585; SEQ ID NO: 1586; SEQ ID NO: 1587; SEQ ID NO: 1588; SEQ ID NO: 1589; SEQ ID NO: 1590; SEQ ID NO: 1591; SEQ ID NO: 1592; SEQ ID NO: 1593; SEQ ID NO: 1594; SEQ ID NO: 1595; SEQ ID NO: 1596; SEQ ID NO: 1597; SEQ ID NO: 1598; SEQ ID NO: 1599; SEQ ID NO: 1600; SEQ ID NO: 1601; SEQ ID NO: 1602; SEQ ID NO: 1603; SEQ ID NO: 1604; SEQ ID NO: 1605; SEQ ID NO: 1606; SEQ ID NO: 1607; SEQ ID NO: 1608; SEQ ID NO: 1609; SEQ ID NO: 1610; SEQ ID NO: 1611; SEQ ID NO: 1612; SEQ ID NO: 1613; SEQ ID NO: 1614; SEQ ID NO: 1615; SEQ ID NO: 1616; SEQ ID NO: 1617; SEQ ID NO: 1618; SEQ ID NO: 1619; SEQ ID NO: 1620; SEQ ID NO: 1621; SEQ ID NO: 1622; SEQ ID NO: 1623; SEQ ID NO: 1624; SEQ ID NO: 1625; SEQ ID NO: 1626; SEQ ID NO: 1627; SEQ ID NO: 1628; SEQ ID NO: 1629; SEQ ID NO: 1630; SEQ ID NO: 1631; SEQ ID NO: 1632; SEQ ID NO: 1633; SEQ ID NO: 1634; SEQ ID NO: 1635; SEQ ID NO: 1636; SEQ ID NO: 1637; SEQ ID NO: 1638; SEQ ID NO: 1639; SEQ ID NO: 1640; SEQ ID NO: 1641; SEQ ID NO: 1642; SEQ ID NO: 1643; SEQ ID NO: 1644; SEQ ID NO: 1645; SEQ ID NO: 1646; SEQ ID NO: 1647; SEQ ID NO: 1648; SEQ ID NO: 1649; SEQ ID NO: 1650; SEQ ID NO: 1651; SEQ ID NO: 1652; SEQ ID NO: 1653; SEQ ID NO: 1654; SEQ ID NO: 1655; SEQ ID NO: 1656; SEQ ID NO: 1657; SEQ ID NO: 1658; SEQ ID NO: 1659; SEQ ID NO: 1660; SEQ ID NO: 1661; SEQ ID NO: 1662; SEQ ID NO: 1663; SEQ ID NO: 1664; SEQ ID NO: 1665; SEQ ID NO: 1666; SEQ ID NO: 1667; SEQ ID NO: 1668; SEQ ID NO: 1669; SEQ ID NO: 1670; SEQ ID NO: 1671; SEQ ID NO: 1672; SEQ ID NO: 1673; SEQ ID NO: 1674; SEQ ID NO: 1675; SEQ ID NO: 1676; SEQ ID NO: 1677; SEQ ID NO: 1678; SEQ ID NO: 1679; SEQ ID NO: 1680; SEQ ID NO: 1681; SEQ ID NO: 1682; SEQ ID NO: 1683; SEQ ID NO: 1684; SEQ ID NO: 1685; SEQ ID NO: 1686; SEQ ID NO: 1687; SEQ ID NO: 1688; SEQ ID NO: 1689; SEQ ID NO: 1690; SEQ ID NO: 1691; SEQ ID NO: 1692; SEQ ID NO: 1693; SEQ ID NO: 1694; SEQ ID NO: 1695; SEQ ID NO: 1696; SEQ ID NO: 1697; SEQ ID NO: 1698; SEQ ID NO: 1699; SEQ ID NO: 1700; SEQ ID NO: 1701; SEQ ID NO: 1702; SEQ ID NO: 1703; SEQ ID NO: 1704; SEQ ID NO: 1705; SEQ ID NO: 1706; SEQ ID NO: 1707; SEQ ID NO: 1708; SEQ ID NO: 1709; SEQ ID NO: 1710; SEQ ID NO: 1711; SEQ ID NO: 1712; SEQ ID NO: 1713; SEQ ID NO: 1714; SEQ ID NO: 1715; SEQ ID NO: 1716; SEQ ID NO: 1717; SEQ ID NO: 1718; SEQ ID NO: 1719; SEQ ID NO: 1720; SEQ ID NO: 1721; SEQ ID NO: 1722; SEQ ID NO: 1723; SEQ ID NO: 1724; SEQ ID NO: 1725; SEQ ID NO: 9593 and SEQ ID NO: 9594.

In another embodiment, the H. pylori cytoplasmic polypeptide or a fragment thereof is an H. pylori polypeptide involved in amino acid metabolism or a fragment thereof encoded by a nucleic acid having a sequence selected from the group consisting of SEQ ID NO: 1726; SEQ ID NO: 1727; SEQ ID NO: 1728; SEQ ID NO: 1729; SEQ ID NO: 1730; SEQ ID NO: 1731; SEQ ID NO: 1732; SEQ ID NO: 1733; SEQ ID NO: 1734; SEQ ID NO: 1735; SEQ ID NO: 1736; SEQ ID NO: 1737; SEQ ID NO: 1738; SEQ ID NO: 1739; SEQ ID NO: 1740; SEQ ID NO: 1741; SEQ ID NO: 1742; SEQ ID NO: 1743; SEQ ID NO: 1744; SEQ ID NO: 1745; SEQ ID NO: 1746; SEQ ID NO: 1747; SEQ ID NO: 1748; SEQ ID NO: 1749; SEQ ID NO: 1750; SEQ ID NO: 1751; SEQ ID NO: 1752; SEQ ID NO: 1753; SEQ ID NO: 1754; SEQ ID NO: 1755; SEQ ID NO: 1756; SEQ ID NO: 1757; SEQ ID NO: 1758; SEQ ID NO: 1759; SEQ ID NO: 1760; SEQ ID NO: 1761; SEQ ID NO: 1762; SEQ ID NO: 1763; SEQ ID NO: 1764; SEQ ID NO: 1765; SEQ ID NO: 1766; SEQ ID NO: 1767; SEQ ID NO: 1768; SEQ ID NO: 1769; SEQ ID NO: 1770; SEQ ID NO: 1771; SEQ ID NO: 1772; SEQ ID NO: 1773; SEQ ID NO: 1774; SEQ ID NO: 1775; SEQ ID NO: 1776; SEQ ID NO: 1777; SEQ ID NO: 1778; SEQ ID NO: 1779; SEQ ID NO: 1780; SEQ ID NO: 1781; SEQ ID NO: 1782; SEQ ID NO: 1783; SEQ ID NO: 1784; SEQ ID NO: 1785; SEQ ID NO: 1786; SEQ ID NO: 1787; SEQ ID NO: 1788; SEQ ID NO: 1789; SEQ ID NO: 1790; SEQ ID NO: 1791; SEQ ID NO: 1792; SEQ ID NO: 1793; SEQ ID NO: 1794; SEQ ID NO: 1795; SEQ ID NO: 1796; SEQ ID NO: 1797; SEQ ID NO: 1798; SEQ ID NO: 1799; SEQ ID NO: 1800; SEQ ID NO: 1801; SEQ ID NO: 1802; SEQ ID NO: 1803; SEQ ID NO: 1804; SEQ ID NO: 1805; SEQ ID NO: 1806; SEQ ID NO: 1807; SEQ ID NO: 1808; SEQ ID NO: 1809; SEQ ID NO: 1810; SEQ ID NO: 1811; SEQ ID NO: 1812; SEQ ID NO: 1813; SEQ ID NO: 1814; SEQ ID NO: 1815; SEQ ID NO: 1816; SEQ ID NO: 1817; SEQ ID NO: 1818; SEQ ID NO: 1819; SEQ ID NO: 1820; SEQ ID NO: 1821; SEQ ID NO: 1822; SEQ ID NO: 1823; SEQ ID NO: 1824; SEQ ID NO: 1825; SEQ ID NO: 1826; SEQ ID NO: 1827; SEQ ID NO: 1828; SEQ ID NO: 1829; SEQ ID NO: 1830; SEQ ID NO: 1831; SEQ ID NO: 1832; SEQ ID NO: 1833; SEQ ID NO: 1834; SEQ ID NO: 1835; SEQ ID NO: 1836; SEQ ID NO: 1837; SEQ ID NO: 1838; SEQ ID NO: 1839; SEQ ID NO: 1840; SEQ ID NO: 1841; SEQ ID NO: 1842; SEQ ID NO: 1843; SEQ ID NO: 1844; SEQ ID NO: 1845; SEQ ID NO: 1846; SEQ ID NO: 1847; SEQ ID NO: 1848; SEQ ID NO: 1849; SEQ ID NO: 1850; SEQ ID NO: 1851; SEQ ID NO: 1852; SEQ ID NO: 1853; SEQ ID NO: 1854; SEQ ID NO: 1855; SEQ ID NO: 1856; SEQ ID NO: 1857; SEQ ID NO: 1858; SEQ ID NO: 1859; SEQ ID NO: 1860; SEQ ID NO: 1861; SEQ ID NO: 1862; SEQ ID NO: 1863; SEQ ID NO: 1864; SEQ ID NO: 1865; SEQ ID NO: 1866; SEQ ID NO: 1867; SEQ ID NO: 1868; SEQ ID NO: 9595 and SEQ ID NO: 9596.

In another embodiment, the H. pylori cytoplasmic polypeptide or a fragment thereof is an H. pylori polypeptide involved in cofactor metabolism or a fragment thereof encoded by a nucleic acid having a sequence selected from the group consisting of SEQ ID NO: 1869; SEQ ID NO: 1870; SEQ ID NO: 1871; SEQ ID NO: 1872; SEQ ID NO: 1873; SEQ ID NO: 1874; SEQ ID NO: 1875; SEQ ID NO: 1876; SEQ ID NO: 1877; SEQ ID NO: 1878; SEQ ID NO: 1879; SEQ ID NO: 1880; SEQ ID NO: 1881; SEQ ID NO: 1882; SEQ ID NO: 1883; SEQ ID NO: 1884; SEQ ID NO: 1885; SEQ ID NO: 1886; SEQ ID NO: 1887; SEQ ID NO: 1888; SEQ ID NO: 1889; SEQ ID NO: 1890; SEQ ID NO: 1891; SEQ ID NO: 1892; SEQ ID NO: 1893; SEQ ID NO: 1894; SEQ ID NO: 1895; SEQ ID NO: 1896; SEQ ID NO: 1897; SEQ ID NO: 1898; SEQ ID NO: 1899; SEQ ID NO: 1900; SEQ ID NO: 1901; SEQ ID NO: 1902; SEQ ID NO: 1903; SEQ ID NO: 1904; SEQ ID NO: 1905; SEQ ID NO: 1906; SEQ ID NO: 1907; SEQ ID NO: 1908; SEQ ID NO: 1909; SEQ ID NO: 1910; SEQ ID NO: 1911; SEQ ID NO: 1912; SEQ ID NO: 1913; SEQ ID NO: 1914; SEQ ID NO: 1915; SEQ ID NO: 1916; SEQ ID NO: 1917; SEQ ID NO: 1918; SEQ ID NO: 1919; SEQ ID NO: 1920; SEQ ID NO: 1921; SEQ ID NO: 1922; SEQ ID NO: 1923; SEQ ID NO: 1924; SEQ ID NO: 1925; SEQ ID NO: 1926; SEQ ID NO: 1927; SEQ ID NO: 1928; SEQ ID NO: 1929; SEQ ID NO: 1930; SEQ ID NO: 1931; SEQ ID NO: 1932; SEQ ID NO: 1933; SEQ ID NO: 1934; SEQ ID NO: 1935; SEQ ID NO: 1936; SEQ ID NO: 1937; SEQ ID NO: 1938; SEQ ID NO: 1939; SEQ ID NO: 1940; SEQ ID NO: 1941; SEQ ID NO: 1942; SEQ ID NO: 1943; SEQ ID NO: 1944; SEQ ID NO: 1945; SEQ ID NO: 1946; SEQ ID NO: 1947; SEQ ID NO: 1948; SEQ ID NO: 1949; SEQ ID NO: 1950; SEQ ID NO: 1951; SEQ ID NO: 1952; SEQ ID NO: 1953; SEQ ID NO: 1954; SEQ ID NO: 1955; SEQ ID NO: 1956; SEQ ID NO: 1957; SEQ ID NO: 1958; SEQ ID NO: 1959; SEQ ID NO: 1960; SEQ ID NO: 1961; SEQ ID NO: 1962; SEQ ID NO: 1963; SEQ ID NO: 1964; SEQ ID NO: 1965; SEQ ID NO: 1966; SEQ ID NO: 1967; SEQ ID NO: 1968; SEQ ID NO: 1969; SEQ ID NO: 1970; SEQ ID NO: 1971; SEQ ID NO: 1972; SEQ ID NO: 1973; SEQ ID NO: 1974; SEQ ID NO: 1975; SEQ ID NO: 1976; SEQ ID NO: 1977; SEQ ID NO: 1978; SEQ ID NO: 1979; SEQ ID NO: 1980; SEQ ID NO: 1981; SEQ ID NO: 1982; SEQ ID NO: 1983; SEQ ID NO: 1984; SEQ ID NO: 1985; SEQ ID NO: 1986; SEQ ID NO: 1987; SEQ ID NO: 1988; SEQ ID NO: 1989; SEQ ID NO: 1990; SEQ ID NO: 1991; SEQ ID NO: 1992; SEQ ID NO: 1993; SEQ ID NO: 1994; SEQ ID NO: 1995; SEQ ID NO: 1996; SEQ ID NO: 1997; SEQ ID NO: 1998; SEQ ID NO: 1999; SEQ ID NO: 2000; SEQ ID NO: 2001; SEQ ID NO: 2002; SEQ ID NO: 2003; SEQ ID NO: 2004; SEQ ID NO: 2005; SEQ ID NO: 9597.

In another embodiment, the H. pylori cytoplasmic polypeptide or a fragment thereof is an H. pylori polypeptide involved in carbohydrate metabolism or a fragment thereof encoded by a nucleic acid having a sequence selected from the group consisting of SEQ ID NO: 2006; SEQ ID NO: 2007; SEQ ID NO: 2008; SEQ ID NO: 2009; SEQ ID NO: 2010; SEQ ID NO: 2011; SEQ ID NO: 2012; SEQ ID NO: 2013; SEQ ID NO: 2014; SEQ ID NO: 2015; SEQ ID NO: 2016; SEQ ID NO: 2017; SEQ ID NO: 2018; SEQ ID NO: 2019; SEQ ID NO: 2020; SEQ ID NO: 2021; SEQ ID NO: 2022; SEQ ID NO: 2023; SEQ ID NO: 2024; SEQ ID NO: 2025; SEQ ID NO: 2026; SEQ ID NO: 2027; SEQ ID NO: 2028; SEQ ID NO: 2029; SEQ ID NO: 2030; SEQ ID NO: 2031; SEQ ID NO: 2032; SEQ ID NO: 2033; SEQ ID NO: 2034; SEQ ID NO: 2035; SEQ ID NO: 2036; SEQ ID NO: 2037; SEQ ID NO: 2038; SEQ ID NO: 2039; SEQ ID NO: 2040; SEQ ID NO: 2041; SEQ ID NO: 2042; SEQ ID NO: 2043; SEQ ID NO: 2044; SEQ ID NO: 2045; SEQ ID NO: 2046; SEQ ID NO: 2047; SEQ ID NO: 2048; SEQ ID NO: 2049; SEQ ID NO: 2050; SEQ ID NO: 2051; SEQ ID NO: 2052; SEQ ID NO: 2053; SEQ ID NO: 2054; SEQ ID NO: 2055; SEQ ID NO: 2056; SEQ ID NO: 2057; SEQ ID NO: 2058; SEQ ID NO: 2059; SEQ ID NO: 2060; SEQ ID NO: 2061; SEQ ID NO: 2062; SEQ ID NO: 2063; SEQ ID NO: 2064; SEQ ID NO: 2065; SEQ ID NO: 2066; SEQ ID NO: 2067; SEQ ID NO: 2068; SEQ ID NO: 2069; SEQ ID NO: 2070; SEQ ID NO: 2071; SEQ ID NO: 2072; SEQ ID NO: 2073; SEQ ID NO: 2074; SEQ ID NO: 2075; SEQ ID NO: 2076; SEQ ID NO: 9598; SEQ ID NO: 9599.

In another embodiment, the H. pylori cytoplasmic polypeptide or a fragment thereof is an H. pylori polypeptide involved in nucleic acid metabolism or a fragment thereof encoded by a nucleic acid having a nucleotide sequence selected from the group consisting of SEQ ID NO: 2077; SEQ ID NO: 2078; SEQ ID NO: 2079; SEQ ID NO: 2080; SEQ ID NO: 2081; SEQ ID NO: 2082; SEQ ID NO: 2083; SEQ ID NO: 2084; SEQ ID NO: 2085; SEQ ID NO: 2086; SEQ ID NO: 2087; SEQ ID NO: 2088; SEQ ID NO: 2089; SEQ ID NO: 2090; SEQ ID NO: 2091; SEQ ID NO: 2092; SEQ ID NO: 2093; SEQ ID NO: 2094; SEQ ID NO: 2095; SEQ ID NO: 2096; SEQ ID NO: 2097; SEQ ID NO: 2098; SEQ ID NO: 2099; SEQ ID NO: 2100; SEQ ID NO: 2101; SEQ ID NO: 2102; SEQ ID NO: 2103; SEQ ID NO: 2104; SEQ ID NO: 2105; SEQ ID NO: 2106; SEQ ID NO: 2107; SEQ ID NO: 2108; SEQ ID NO: 2109; SEQ ID NO: 2110; SEQ ID NO: 2111; SEQ ID NO: 2112; SEQ ID NO: 2113; SEQ ID NO: 2114; SEQ ID NO: 2115; SEQ ID NO: 2116; SEQ ID NO: 2117; SEQ ID NO: 2118; SEQ ID NO: 2119; SEQ ID NO: 2120; SEQ ID NO: 2121; SEQ ID NO: 2122; SEQ ID NO: 2123; SEQ ID NO: 2124; SEQ ID NO: 2125; SEQ ID NO: 2126; SEQ ID NO: 2127; SEQ ID NO: 2128; SEQ ID NO: 2129; SEQ ID NO: 2130; SEQ ID NO: 2131; SEQ ID NO: 2132; SEQ ID NO: 2133; SEQ ID NO: 2134; SEQ ID NO: 2135; SEQ ID NO: 2136; SEQ ID NO: 2137; SEQ ID NO: 2138; SEQ ID NO: 2139; SEQ ID NO: 2140; SEQ ID NO: 2141; SEQ ID NO: 2142; SEQ ID NO: 2143; SEQ ID NO: 2144; SEQ ID NO: 2145; SEQ ID NO: 2146; SEQ ID NO: 2147; SEQ ID NO: 2148; SEQ ID NO: 2149; SEQ ID NO: 2150; SEQ ID NO: 2151; SEQ ID NO: 2152; SEQ ID NO: 2153; SEQ ID NO: 2154; SEQ ID NO: 2155; SEQ ID NO: 2156; SEQ ID NO: 2157; SEQ ID NO: 2158; SEQ ID NO: 2159; SEQ ID NO: 2160; SEQ ID NO: 2161; SEQ ID NO: 2162; SEQ ID NO: 2163; SEQ ID NO: 2164; SEQ ID NO: 2165; SEQ ID NO: 2166; SEQ ID NO: 2167; SEQ ID NO: 2168; SEQ ID NO: 2169; SEQ ID NO: 2170; SEQ ID NO: 2171; SEQ ID NO: 2172; SEQ ID NO: 2173; SEQ ID NO: 2174; SEQ ID NO: 2175; SEQ ID NO: 2176; SEQ ID NO: 2177; SEQ ID NO: 2178; SEQ ID NO: 2179; SEQ ID NO: 2180; SEQ ID NO: 2181; SEQ ID NO: 2182; SEQ ID NO: 9600.

In another embodiment, the H. pylori cytoplasmic polypeptide or a fragment thereof is an H. pylori polypeptide involved in lipid metabolism or a fragment thereof encoded by a nucleic acid having a nucleotide sequence selected from the group consisting of SEQ ID NO: 2183; SEQ ID NO: 2184; SEQ ID NO: 2185; SEQ ID NO: 2186; SEQ ID NO: 2187; SEQ ID NO: 2188; SEQ ID NO: 2189; SEQ ID NO: 2190; SEQ ID NO: 2191; SEQ ID NO: 2192; SEQ ID NO: 2193; SEQ ID NO: 2194; SEQ ID NO: 2195; SEQ ID NO: 2196; SEQ ID NO: 2197; SEQ ID NO: 2198; SEQ ID NO: 2199; SEQ ID NO: 2200; SEQ ID NO: 2201; SEQ ID NO: 2202; SEQ ID NO: 2203; SEQ ID NO: 2204; SEQ ID NO: 2205; SEQ ID NO: 2206; SEQ ID NO: 2207; SEQ ID NO: 2208; SEQ ID NO: 2209; SEQ ID NO: 2210; SEQ ID NO: 2211; SEQ ID NO: 2212; SEQ ID NO: 2213; SEQ ID NO: 2214; SEQ ID NO: 2215; SEQ ID NO: 2216; SEQ ID NO: 2217; SEQ ID NO: 2218; SEQ ID NO: 9601 and SEQ ID NO: 9602.

In another embodiment, the H. pylori cytoplasmic polypeptide or a fragment thereof is an H. pylori polypeptide involved in mRNA translation and ribosome biogenesis or a fragment thereof encoded by a nucleic acid having a sequence selected from the group consisting of SEQ ID NO: 2219; SEQ ID NO: 2220; SEQ ID NO: 2221; SEQ ID NO: 2222; SEQ ID NO: 2223; SEQ ID NO: 2224; SEQ ID NO: 2225; SEQ ID NO: 2226; SEQ ID NO: 2227; SEQ ID NO: 2228; SEQ ID NO: 2229; SEQ ID NO: 2230; SEQ ID NO: 2231; SEQ ID NO: 2232; SEQ ID NO: 2233; SEQ ID NO: 2234; SEQ ID NO: 2235; SEQ ID NO: 2236; SEQ ID NO: 2237; SEQ ID NO: 2238; SEQ ID NO: 2239; SEQ ID NO: 2240; SEQ ID NO: 2241; SEQ ID NO: 2242; SEQ ID NO: 2243; SEQ ID NO: 2244; SEQ ID NO: 2245; SEQ ID NO: 2246; SEQ ID NO: 2247; SEQ ID NO: 2248; SEQ ID NO: 2249; SEQ ID NO: 2250; SEQ ID NO: 2251; SEQ ID NO: 2252; SEQ ID NO: 2253; SEQ ID NO: 2254; SEQ ID NO: 2255; SEQ ID NO: 2256; SEQ ID NO: 2257; SEQ ID NO: 2258; SEQ ID NO: 2259; SEQ ID NO: 2260; SEQ ID NO: 2261; SEQ ID NO: 2262; SEQ ID NO: 2263; SEQ ID NO: 2264; SEQ ID NO: 2265; SEQ ID NO: 2266; SEQ ID NO: 2267; SEQ ID NO: 2268; SEQ ID NO: 2269; SEQ ID NO: 2270; SEQ ID NO: 2271; SEQ ID NO: 2272; SEQ ID NO: 2273; SEQ ID NO: 2274; SEQ ID NO: 2275; SEQ ID NO: 2276; SEQ ID NO: 2277; SEQ ID NO: 2278; SEQ ID NO: 2279; SEQ ID NO: 2280; SEQ ID NO: 2281; SEQ ID NO: 2282; SEQ ID NO: 2283; SEQ ID NO: 2284; SEQ ID NO: 2285; SEQ ID NO: 2286; SEQ ID NO: 2287; SEQ ID NO: 2288; SEQ ID NO: 2289; SEQ ID NO: 2290; SEQ ID NO: 2291; SEQ ID NO: 2292; SEQ ID NO: 2293; SEQ ID NO: 2294; SEQ ID NO: 2295; SEQ ID NO: 2296; SEQ ID NO: 2297; SEQ ID NO: 2298; SEQ ID NO: 2299; SEQ ID NO: 2300; SEQ ID NO: 2301; SEQ ID NO: 2302; SEQ ID NO: 2303; SEQ ID NO: 2304; SEQ ID NO: 2305; SEQ ID NO: 2306; SEQ ID NO: 2307; SEQ ID NO: 2308; SEQ ID NO: 2309; SEQ ID NO: 2310; SEQ ID NO: 2311; SEQ ID NO: 2312; SEQ ID NO: 2313; SEQ ID NO: 2314; SEQ ID NO: 2315; SEQ ID NO: 2316; SEQ ID NO: 2317; SEQ ID NO: 2318; SEQ ID NO: 2319; SEQ ID NO: 2320; SEQ ID NO: 2321; SEQ ID NO: 2322; SEQ ID NO: 2323; SEQ ID NO: 2324; SEQ ID NO: 2325; SEQ ID NO: 2326; SEQ ID NO: 2327; SEQ ID NO: 2328; SEQ ID NO: 2329; SEQ ID NO: 2330; SEQ ID NO: 2331; SEQ ID NO: 2332; SEQ ID NO: 2333; SEQ ID NO: 2334; SEQ ID NO: 2335; SEQ ID NO: 2336; SEQ ID NO: 2337; SEQ ID NO: 2338; SEQ ID NO: 2339; SEQ ID NO: 2340; SEQ ID NO: 2341; SEQ ID NO: 2342; SEQ ID NO: 2343; SEQ ID NO: 2344; SEQ ID NO: 2345; SEQ ID NO: 2346; SEQ ID NO: 2347; SEQ ID NO: 2348; SEQ ID NO: 2349; SEQ ID NO: 2350; SEQ ID NO: 2351; SEQ ID NO: 2352; SEQ ID NO: 2353; SEQ ID NO: 2354; SEQ ID NO: 2355; SEQ ID NO: 2356; SEQ ID NO: 2357; SEQ ID NO: 2358; SEQ ID NO: 2359; SEQ ID NO: 2360; SEQ ID NO: 2361; SEQ ID NO: 2362; SEQ ID NO: 2363; SEQ ID NO: 2364; SEQ ID NO: 2365; SEQ ID NO: 2366; SEQ ID NO: 2367; SEQ ID NO: 2368; SEQ ID NO: 2369; SEQ ID NO: 2370; SEQ ID NO: 2371; SEQ ID NO: 2372; SEQ ID NO: 2373; SEQ ID NO: 2374; SEQ ID NO: 2375; SEQ ID NO: 2376; SEQ ID NO: 2377; SEQ ID NO: 2378; SEQ ID NO: 2379; SEQ ID NO: 2380; SEQ ID NO: 2381; SEQ ID NO: 2382; SEQ ID NO: 2383; SEQ ID NO: 2384; SEQ ID NO: 2385; SEQ ID NO: 2386; SEQ ID NO: 2387; SEQ ID NO: 2388; SEQ ID NO: 2389; SEQ ID NO: 2390; SEQ ID NO: 2391; SEQ ID NO: 2392; SEQ ID NO: 2393; SEQ ID NO: 2394; SEQ ID NO: 2395; SEQ ID NO: 2396; SEQ ID NO: 2397; SEQ ID NO: 2398; SEQ ID NO: 2399; SEQ ID NO: 2400; SEQ ID NO: 2401; SEQ ID NO: 2402; SEQ ID NO: 2403; SEQ ID NO: 2404; SEQ ID NO: 2405; SEQ ID NO: 2406; SEQ ID NO: 2407; SEQ ID NO: 2408; SEQ ID NO: 2409; SEQ ID NO: 2410; SEQ ID NO: 2411; SEQ ID NO: 2412; SEQ ID NO: 2413; SEQ ID NO: 2414; SEQ ID NO: 2415; SEQ ID NO: 2416; SEQ ID NO: 2417; SEQ ID NO: 2418; SEQ ID NO: 2419; SEQ ID NO: 2420; SEQ ID NO: 2421; SEQ ID NO: 2422; SEQ ID NO: 2423; SEQ ID NO: 2424; SEQ ID NO: 2425; SEQ ID NO: 2426; SEQ ID NO: 2427; SEQ ID NO: 2428; SEQ ID NO: 2429; SEQ ID NO: 2430; SEQ ID NO: 2431; SEQ ID NO: 2432; SEQ ID NO: 2433; SEQ ID NO: 2434; SEQ ID NO: 2435; SEQ ID NO: 2436; SEQ ID NO: 2437; SEQ ID NO: 2438; SEQ ID NO: 9603 and SEQ ID NO: 9604.

In another embodiment, the H. pylori cytoplasmic polypeptide or a fragment thereof is an H. pylori polypeptide involved in genome replication, transcription, recombination and repair or a fragment thereof encoded by a nucleic acid having a nucleotide sequence selected from the group consisting of SEQ ID NO: 2439; SEQ ID NO: 2440; SEQ ID NO: 2441; SEQ ID NO: 2442; SEQ ID NO: 2443; SEQ ID NO: 2444; SEQ ID NO: 2445; SEQ ID NO: 2446; SEQ ID NO: 2447; SEQ ID NO: 2448; SEQ ID NO: 2449; SEQ ID NO: 2450; SEQ ID NO: 2451; SEQ ID NO: 2452; SEQ ID NO: 2453; SEQ ID NO: 2454; SEQ ID NO: 2455; SEQ ID NO: 2456; SEQ ID NO: 2457; SEQ ID NO: 2458; SEQ ID NO: 2459; SEQ ID NO: 2460; SEQ ID NO: 2461; SEQ ID NO: 2462; SEQ ID NO: 2463; SEQ ID NO: 2464; SEQ ID NO: 2465; SEQ ID NO: 2466; SEQ ID NO: 2467; SEQ ID NO: 2468; SEQ ID NO: 2469; SEQ ID NO: 2470; SEQ ID NO: 2471; SEQ ID NO: 2472; SEQ ID NO: 2473; SEQ ID NO: 2474; SEQ ID NO: 2475; SEQ ID NO: 2476; SEQ ID NO: 2477; SEQ ID NO: 2478; SEQ ID NO: 2479; SEQ ID NO: 2480; SEQ ID NO: 2481; SEQ ID NO: 2482; SEQ ID NO: 2483; SEQ ID NO: 2484; SEQ ID NO: 2485; SEQ ID NO: 2486; SEQ ID NO: 2487; SEQ ID NO: 2488; SEQ ID NO: 2489; SEQ ID NO: 2490; SEQ ID NO: 2491; SEQ ID NO: 2492; SEQ ID NO: 2493; SEQ ID NO: 2494; SEQ ID NO: 2495; SEQ ID NO: 2496; SEQ ID NO: 2497; SEQ ID NO: 2498; SEQ ID NO: 2499; SEQ ID NO: 2500; SEQ ID NO: 2501; SEQ ID NO: 2502; SEQ ID NO: 2503; SEQ ID NO: 2504; SEQ ID NO: 2505; SEQ ID NO: 2506; SEQ ID NO: 2507; SEQ ID NO: 2508; SEQ ID NO: 2509; SEQ ID NO: 2510; SEQ ID NO: 2511; SEQ ID NO: 2512; SEQ ID NO: 2513; SEQ ID NO: 2514; SEQ ID NO: 2515; SEQ ID NO: 2516; SEQ ID NO: 2517; SEQ ID NO: 2518; SEQ ID NO: 2519; SEQ ID NO: 2520; SEQ ID NO: 2521; SEQ ID NO: 2522; SEQ ID NO: 2523; SEQ ID NO: 2524; SEQ ID NO: 2525; SEQ ID NO: 2526; SEQ ID NO: 2527; SEQ ID NO: 2528; SEQ ID NO: 2529; SEQ ID NO: 2530; SEQ ID NO: 2531; SEQ ID NO: 2532; SEQ ID NO: 2533; SEQ ID NO: 2534; SEQ ID NO: 2535; SEQ ID NO: 2536; SEQ ID NO: 2537; SEQ ID NO: 2538; SEQ ID NO: 2539; SEQ ID NO: 2540; SEQ ID NO: 2541; SEQ ID NO: 2542; SEQ ID NO: 2543; SEQ ID NO: 2544; SEQ ID NO: 2545; SEQ ID NO: 2546; SEQ ID NO: 2547; SEQ ID NO: 2548; SEQ ID NO: 2549; SEQ ID NO: 2550; SEQ ID NO: 2551; SEQ ID NO: 2552; SEQ ID NO: 2553; SEQ ID NO: 2554; SEQ ID NO: 2555; SEQ ID NO: 2556; SEQ ID NO: 2557; SEQ ID NO: 2558; SEQ ID NO: 2559; SEQ ID NO: 2560; SEQ ID NO: 2561; SEQ ID NO: 2562; SEQ ID NO: 2563; SEQ ID NO: 2564; SEQ ID NO: 2565; SEQ ID NO: 2566; SEQ ID NO: 2567; SEQ ID NO: 2568; SEQ ID NO: 2569; SEQ ID NO: 2570; SEQ ID NO: 2571; SEQ ID NO: 2572; SEQ ID NO: 2573; SEQ ID NO: 2574; SEQ ID NO: 2575; SEQ ID NO: 2576; SEQ ID NO: 2577; SEQ ID NO: 2578; SEQ ID NO: 2579; SEQ ID NO: 2580; SEQ ID NO: 2581; SEQ ID NO: 2582; SEQ ID NO: 2583; SEQ ID NO: 2584; SEQ ID NO: 2585; SEQ ID NO: 2586; SEQ ID NO: 2587; SEQ ID NO: 2588; SEQ ID NO: 2589; SEQ ID NO: 2590; SEQ ID NO: 2591; SEQ ID NO: 2592; SEQ ID NO: 2593; SEQ ID NO: 2594; SEQ ID NO: 2595; SEQ ID NO: 2596; SEQ ID NO: 2597; SEQ ID NO: 2598; SEQ ID NO: 2599; SEQ ID NO: 2600; SEQ ID NO: 2601; SEQ ID NO: 2602; SEQ ID NO: 2603; SEQ ID NO: 2604; SEQ ID NO: 2605; SEQ ID NO: 2606; SEQ ID NO: 2607; SEQ ID NO: 2608; SEQ ID NO: 2609; SEQ ID NO: 2610; SEQ ID NO: 2611; SEQ ID NO: 2612; SEQ ID NO: 2613; SEQ ID NO: 2614; SEQ ID NO: 2615; SEQ ID NO: 2616; SEQ ID NO: 2617; SEQ ID NO: 2618; SEQ ID NO: 2619; SEQ ID NO: 2620; SEQ ID NO: 2621; SEQ ID NO: 2622; SEQ ID NO: 2623; SEQ ID NO: 2624; SEQ ID NO: 2625; SEQ ID NO: 2626; SEQ ID NO: 2627; SEQ ID NO: 2628; SEQ ID NO: 2629; SEQ ID NO: 2630; SEQ ID NO: 2631; SEQ ID NO: 2632; SEQ ID NO: 2633; SEQ ID NO: 2634; SEQ ID NO: 2635; SEQ ID NO: 2636; SEQ ID NO: 2637; SEQ ID NO: 2638; SEQ ID NO: 2639; SEQ ID NO: 2640; SEQ ID NO: 2641; SEQ ID NO: 2642; SEQ ID NO: 2643; SEQ ID NO: 2644; SEQ ID NO: 2645; SEQ ID NO: 2646; SEQ ID NO: 2647; SEQ ID NO: 2648; SEQ ID NO: 2649; SEQ ID NO: 2650; SEQ ID NO: 2651; SEQ ID NO: 2652; SEQ ID NO: 2653; SEQ ID NO: 2654; SEQ ID NO: 2655; SEQ ID NO: 2656; SEQ ID NO: 2657; SEQ ID NO: 2658; SEQ ID NO: 2659; SEQ ID NO: 2660; SEQ ID NO: 2661; SEQ ID NO: 2662; SEQ ID NO: 2663; SEQ ID NO: 2664; SEQ ID NO: 2665; SEQ ID NO: 2666; SEQ ID NO: 2667; SEQ ID NO: 2668; SEQ ID NO: 2669; SEQ ID NO: 2670; SEQ ID NO: 2671; SEQ ID NO: 2672; SEQ ID NO: 2673; SEQ ID NO: 2674; SEQ ID NO: 2675; SEQ ID NO: 2676; SEQ ID NO: 2677; SEQ ID NO: 2678; SEQ ID NO: 2679; SEQ ID NO: 2680; SEQ ID NO: 2681; SEQ ID NO: 2682; SEQ ID NO: 2683; SEQ ID NO: 2684; SEQ ID NO: 2685; SEQ ID NO: 2686; SEQ ID NO: 2687; SEQ ID NO: 2688; SEQ ID NO: 2689; SEQ ID NO: 2690; SEQ ID NO: 2691; SEQ ID NO: 2692; SEQ ID NO: 2693; SEQ ID NO: 2694; SEQ ID NO: 2695; SEQ ID NO: 2696; SEQ ID NO: 2697; SEQ ID NO: 2698; SEQ ID NO: 2699; SEQ ID NO: 2700; SEQ ID NO: 2701; SEQ ID NO: 2702; SEQ ID NO: 2703; SEQ ID NO: 2704; SEQ ID NO: 2705; SEQ ID NO: 2706; SEQ ID NO: 2707; SEQ ID NO: 2708; SEQ ID NO: 2709; SEQ ID NO: 2710; SEQ ID NO: 2711; SEQ ID NO: 2712; SEQ ID NO: 2713; SEQ ID NO: 2714; SEQ ID NO: 2715; SEQ ID NO: 2716; SEQ ID NO: 2717; SEQ ID NO: 2718; SEQ ID NO: 2719; SEQ ID NO: 2720; SEQ ID NO: 2721; SEQ ID NO: 2722; SEQ ID NO: 2723; SEQ ID NO: 2724; SEQ ID NO: 2725; SEQ ID NO: 2726; SEQ ID NO: 2727; SEQ ID NO: 2728; SEQ ID NO: 2729; SEQ ID NO: 2730; SEQ ID NO: 2731; SEQ ID NO: 2732; SEQ ID NO: 2733; SEQ ID NO: 2734; SEQ ID NO: 2735; SEQ ID NO: 2736; SEQ ID NO: 2737; SEQ ID NO: 2738; SEQ ID NO: 2739; SEQ ID NO: 2740; SEQ ID NO: 2741; SEQ ID NO: 2742; SEQ ID NO: 2743; SEQ ID NO: 2744; SEQ ID NO: 2745; SEQ ID NO: 2746; SEQ ID NO: 2747; SEQ ID NO: 2748; SEQ ID NO: 2749; SEQ ID NO: 2750; SEQ ID NO: 2751; SEQ ID NO: 2752; SEQ ID NO: 2753; SEQ ID NO: 2754; SEQ ID NO: 2755; SEQ ID NO: 2756; SEQ ID NO: 2757; SEQ ID NO: 2758; SEQ ID NO: 2759; SEQ ID NO: 2760; SEQ ID NO: 2761; SEQ ID NO: 2762; SEQ ID NO: 2763; SEQ ID NO: 2764; SEQ ID NO: 2765; SEQ ID NO: 2766; SEQ ID NO: 2767; SEQ ID NO: 2768; SEQ ID NO: 2769; SEQ ID NO: 2770; SEQ ID NO: 2771; SEQ ID NO: 2772; SEQ ID NO: 2773; SEQ ID NO: 2774; SEQ ID NO: 2775; SEQ ID NO: 2776; SEQ ID NO: 2777; SEQ ID NO: 2778; SEQ ID NO: 2779; SEQ ID NO: 2780; SEQ ID NO: 2781; SEQ ID NO: 2782; SEQ ID NO: 2783; SEQ ID NO: 2784; SEQ ID NO: 2785; SEQ ID NO: 2786; SEQ ID NO: 2787; SEQ ID NO: 2788; SEQ ID NO: 2789; SEQ ID NO: 2790; SEQ ID NO: 2791; SEQ ID NO: 2792; SEQ ID NO: 2793; SEQ ID NO: 2794; SEQ ID NO: 2795; SEQ ID NO: 2796; SEQ ID NO: 2797; SEQ ID NO: 2798; SEQ ID NO: 2799; SEQ ID NO: 2800; SEQ ID NO: 2801; SEQ ID NO: 2802; SEQ ID NO: 2803; SEQ ID NO: 2804; SEQ ID NO: 2805; SEQ ID NO: 2806; SEQ ID NO: 2807; SEQ ID NO: 9605; SEQ ID NO: 9606; SEQ ID NO: 9607; SEQ ID NO: 9608.

In another embodiment, the H. pylori cytoplasmic polypeptide or a fragment thereof is an H. pylori polypeptide involved in secretion and adhesion or a fragment thereof encoded by a nucleic acid having a sequence selected from the group consisting of SEQ ID NO: 2808; SEQ ID NO: 2809; SEQ ID NO: 2810; SEQ ID NO: 2811; SEQ ID NO: 2812; SEQ ID NO: 2813; SEQ ID NO: 2814; SEQ ID NO: 2815; SEQ ID NO: 2816; SEQ ID NO: 2817; SEQ ID NO: 2818; SEQ ID NO: 2819; SEQ ID NO: 2820; SEQ ID NO: 2821; SEQ ID NO: 2822; SEQ ID NO: 2823; SEQ ID NO: 2824.

In another embodiment, the H. pylori cytoplasmic polypeptide or a fragment thereof is an H. pylori polypeptide involved in outer membrane and cell wall biogenesis or a fragment thereof encoded by a nucleic acid having a sequence selected from the group consisting of SEQ ID NO: 2825; SEQ ID NO: 2826; SEQ ID NO: 2827; SEQ ID NO: 2828; SEQ ID NO: 2829; SEQ ID NO: 2830; SEQ ID NO: 2831; SEQ ID NO: 2832; SEQ ID NO: 2833; SEQ ID NO: 2834; SEQ ID NO: 2835; SEQ ID NO: 2836; SEQ ID NO: 2837; SEQ ID NO: 2838; SEQ ID NO: 2839; SEQ ID NO: 2840; SEQ ID NO: 2841; SEQ ID NO: 2842; SEQ ID NO: 2843; SEQ ID NO: 2844; SEQ ID NO: 2845; SEQ ID NO: 2846; SEQ ID NO: 2847; SEQ ID NO: 2848; SEQ ID NO: 2849; SEQ ID NO: 2850; SEQ ID NO: 2851; SEQ ID NO: 2852; SEQ ID NO: 2853; SEQ ID NO: 2854; SEQ ID NO: 2855; SEQ ID NO: 2856; SEQ ID NO: 2857; SEQ ID NO: 2858; SEQ ID NO: 2859; SEQ ID NO: 2860; SEQ ID NO: 2861; SEQ ID NO: 2862; SEQ ID NO: 2863; SEQ ID NO: 2864; SEQ ID NO: 2865; SEQ ID NO: 2866; SEQ ID NO: 2867; SEQ ID NO: 2868; SEQ ID NO: 2869; SEQ ID NO: 2870; SEQ ID NO: 2871; SEQ ID NO: 2872; SEQ ID NO: 2873; SEQ ID NO: 2874; SEQ ID NO: 2875; SEQ ID NO: 2876; SEQ ID NO: 2877; SEQ ID NO: 9609; and SEQ ID NO: 9610.

In another embodiment, the H. pylori cytoplasmic polypeptide or a fragment thereof is an H. pylori polypeptide involved in protein folding and stabilization or a fragment thereof encoded by a nucleic acid having a sequence selected from the group consisting of SEQ ID NO:2878; SEQ ID NO: 2879; SEQ ID NO: 2880; SEQ ID NO: 2881; SEQ ID NO: 2882; SEQ ID NO: 2883; SEQ ID NO: 2884; SEQ ID NO: 2885; SEQ ID NO: 2886; SEQ ID NO: 2887; SEQ ID NO: 2888; SEQ ID NO: 2889; SEQ ID NO: 2890; SEQ ID NO: 2891; SEQ ID NO: 2892; SEQ ID NO: 2893; SEQ ID NO: 2894; SEQ ID NO: 2895; SEQ ID NO: 2896; SEQ ID NO: 2897; SEQ ID NO: 2898; SEQ ID NO: 2899; SEQ ID NO: 2900; SEQ ID NO: 2901; SEQ ID NO: 2902; SEQ ID NO: 2903; SEQ ID NO: 2904; SEQ ID NO: 2905; SEQ ID NO: 2906; SEQ ID NO: 2907; SEQ ID NO: 2908; SEQ ID NO: 2909; SEQ ID NO: 2910; SEQ ID NO: 2911; SEQ ID NO: 2912; SEQ ID NO: 2913; SEQ ID NO: 2914; SEQ ID NO: 2915; SEQ ID NO: 2916; SEQ ID NO: 2917; SEQ ID NO: 2918; SEQ ID NO: 9611 and SEQ ID NO: 9612.

Particularly preferred is an isolated H. pylori secreted polypeptide or a fragment thereof, wherein the polypeptide has an amino acid sequence selected from the group consisting of SEQ ID NO: 8778; SEQ ID NO: 79; SEQ ID NO: 8780; SEQ ID NO: 8781; SEQ ID NO: 8782; SEQ ID NO: 8783; SEQ ID NO: 8784; SEQ ID NO: 8785; SEQ ID NO: 8786; SEQ ID NO: 8787; SEQ ID NO: 8788; SEQ ID NO: 8789; SEQ ID NO: 8790; SEQ ID NO: 8791; SEQ ID NO: 8792; SEQ ID NO: 8793; SEQ ID NO: 8794; SEQ ID NO: 8795; SEQ ID NO: 8796; SEQ ID NO: 8797; SEQ ID NO: 8798; SEQ ID NO: 8799; SEQ ID NO: 8800; SEQ ID NO: 8801; SEQ ID NO: 8802; SEQ ID NO: 8803; SEQ ID NO: 8804; SEQ ID NO: 8805; SEQ ID NO: 8806; SEQ ID NO: 8807; SEQ ID NO: 8808; SEQ ID NO: 8809; SEQ ID NO: 8810; SEQ ID NO: 8811; SEQ ID NO: 8812; SEQ ID NO: 8813; SEQ ID NO: 8814; SEQ ID NO: 8815; SEQ ID NO: 8816; SEQ ID NO: 8817; SEQ ID NO: 8818; SEQ ID NO: 8819; SEQ ID NO: 8820; SEQ ID NO: 8821; SEQ ID NO: 8822; SEQ ID NO: 8823; SEQ ID NO: 8824; SEQ ID NO: 8825; SEQ ID NO: 8826; SEQ ID NO: 8827; SEQ ID NO: 8828; SEQ ID NO: 8829; SEQ ID NO: 8830; SEQ ID NO: 8831; SEQ ID NO: 8832; SEQ ID NO: 8833; SEQ ID NO: 8834; SEQ ID NO: 8835; SEQ ID NO: 8836; SEQ ID NO: 8837; SEQ ID NO: 8838; SEQ ID NO: 8839; SEQ ID NO: 8840; SEQ ID NO: 8841; SEQ ID NO:8842; SEQ ID NO:8843; SEQ ID NO:8844; SEQ ID NO:8845; SEQ ID NO: 8846; SEQ ID NO: 8847; SEQ ID NO: 8848; SEQ ID NO: 8849; SEQ ID NO: 8850; SEQ ID NO: 8851; SEQ ID NO: 8852; SEQ ID NO: 8853; SEQ ID NO: 8854; SEQ ID NO: 8855; SEQ ID NO: 8856; SEQ ID NO: 8857; SEQ ID NO: 8858; SEQ ID NO: 8859; SEQ ID NO: 8860; SEQ ID NO: 8861; SEQ ID NO: 8862; SEQ ID NO: 8863; SEQ ID NO: 8864; SEQ ID NO: 8865; SEQ ID NO: 8866; SEQ ID NO: 8867; SEQ ID NO: 8868; SEQ ID NO: 8869; SEQ ID NO: 8870; SEQ ID NO: 8871; SEQ ID NO: 8872; SEQ ID NO: 8873; SEQ ID NO: 8874; SEQ ID NO: 8875; SEQ ID NO: 8876; SEQ ID NO: 8877; SEQ ID NO: 8878; SEQ ID NO: 8879; SEQ ID NO: 8880; SEQ ID NO: 8881; SEQ ID NO: 8882; SEQ ID NO: 8883; SEQ ID NO: 8884; SEQ ID NO: 8885; SEQ ID NO: 8886; SEQ ID NO: 8887; SEQ ID NO: 8888; SEQ ID NO: 8889; SEQ ID NO: 8890; SEQ ID NO: 8891; SEQ ID NO: 8892; SEQ ID NO: 8893; SEQ ID NO: 8894; SEQ ID NO: 8895; SEQ ID NO: 8896; SEQ ID NO: 8897; SEQ ID NO: 8898; SEQ ID NO: 8899; SEQ ID NO: 8900; SEQ ID NO: 8901; SEQ ID NO: 8902; SEQ ID NO: 8903; SEQ ID NO: 8904; SEQ ID NO: 8905; SEQ ID NO: 8906; SEQ ID NO: 8907; SEQ ID NO: 8908; SEQ ID NO: 8909; SEQ ID NO: 8910; SEQ ID NO: 8911; SEQ ID NO: 8912; SEQ ID NO: 8913; SEQ ID NO: 8914; SEQ ID NO: 8915; SEQ ID NO: 8916; SEQ ID NO: 8917; SEQ ID NO: 8918; SEQ ID NO: 8919; SEQ ID NO: 8920; SEQ ID NO: 8921; SEQ ID NO: 8922; SEQ ID NO: 8923; SEQ ID NO: 8924; SEQ ID NO: 8925; SEQ ID NO: 8926; SEQ ID NO: 8927; SEQ ID NO: 8928; SEQ ID NO: 8929; SEQ ID NO: 8930; SEQ ID NO: 8931; SEQ ID NO: 8932; SEQ ID NO: 8933; SEQ ID NO: 8934; SEQ ID NO: 8935; SEQ ID NO: 8936; SEQ ID NO: 8937; SEQ ID NO: 8938; SEQ ID NO: 8939; SEQ ID NO: 8940; SEQ ID NO: 8941; SEQ ID NO: 8942; SEQ ID NO: 8943; SEQ ID NO: 8944; SEQ ID NO: 8945; SEQ ID NO: 8946; SEQ ID NO: 8947; SEQ ID NO: 8948; SEQ ID NO: 8949; SEQ ID NO: 8950; SEQ ID NO: 8951; SEQ ID NO: 8952; SEQ ID NO: 8953; SEQ ID NO: 8954; SEQ ID NO: 8955; SEQ ID NO: 8956; SEQ ID NO: 8957; SEQ ID NO: 8958; SEQ ID NO: 8959; SEQ ID NO: 8960; SEQ ID NO: 8961; SEQ ID NO: 8962; SEQ ID NO: 8963; SEQ ID NO: 8964; SEQ ID NO: 8965; SEQ ID NO: 8966; SEQ ID NO: 8967; SEQ ID NO: 8968; SEQ ID NO: 8969; SEQ ID NO: 8970; SEQ ID NO: 8971; SEQ ID NO: 8972; SEQ ID NO: 8973; SEQ ID NO: 8974; SEQ ID NO: 8975; SEQ ID NO: 8976; SEQ ID NO: 8977; SEQ ID NO: 8978; SEQ ID NO: 8979; SEQ ID NO: 8980; SEQ ID NO: 8981; SEQ ID NO: 8982; SEQ ID NO: 8983; SEQ ID NO: 8984; SEQ ID NO: 8985; SEQ ID NO: 8986; SEQ ID NO: 8987; SEQ ID NO: 8988; SEQ ID NO: 8989; SEQ ID NO: 8990; SEQ ID NO: 8991; SEQ ID NO: 8992; SEQ ID NO: 8993; SEQ ID NO: 8994; SEQ ID NO: 8995; SEQ ID NO: 8996; SEQ ID NO: 8997; SEQ ID NO: 8998; SEQ ID NO: 8999; SEQ ID NO: 9000; SEQ ID NO: 9001; SEQ ID NO: 9002; SEQ ID NO: 9003; SEQ ID NO: 9004; SEQ ID NO: 9005; SEQ ID NO: 9006; SEQ ID NO: 9007; SEQ ID NO: 9008; SEQ ID NO: 9009; SEQ ID NO: 9010; SEQ ID NO: 9011; SEQ ID NO: 9012; SEQ ID NO: 9013; SEQ ID NO: 9014; SEQ ID NO: 9015; SEQ ID NO: 9016; SEQ ID NO: 9017; SEQ ID NO: 9018; SEQ ID NO: 9019; SEQ ID NO: 9020; SEQ ID NO: 9021; SEQ ID NO: 9022; SEQ ID NO: 9023; SEQ ID NO: 9024; SEQ ID NO: 9025; SEQ ID NO: 9026; SEQ ID NO: 9027; SEQ ID NO: 9028; SEQ ID NO: 9029; SEQ ID NO: 9030; SEQ ID NO: 9031; SEQ ID NO: 9032; SEQ ID NO: 9033; SEQ ID NO: 9034; SEQ ID NO: 9035; SEQ ID NO: 9036; SEQ ID NO: 9037; SEQ ID NO: 9038; SEQ ID NO: 9039; SEQ ID NO: 9040; SEQ ID NO: 9041; SEQ ID NO: 9042; SEQ ID NO: 9043; SEQ ID NO: 9044; SEQ ID NO: 9045; SEQ ID NO: 9046; SEQ ID NO: 9047; SEQ ID NO: 9048; SEQ ID NO: 9049; SEQ ID NO: 9050; SEQ ID NO: 9051; SEQ ID NO: 9052; SEQ ID NO: 9053; SEQ ID NO: 9054; SEQ ID NO: 9055; SEQ ID NO: 9056; SEQ ID NO: 9057; SEQ ID NO: 9058; SEQ ID NO: 9059; SEQ ID NO: 9060; SEQ ID NO: 9061; SEQ ID NO: 9062; SEQ ID NO: 9063; SEQ ID NO: 9064; SEQ ID NO: 9065; SEQ ID NO: 9066; SEQ ID NO: 9067; SEQ ID NO: 9068; SEQ ID NO: 9069; SEQ ID NO: 9070; SEQ ID NO: 9071; SEQ ID NO: 9072; SEQ ID NO: 9073; SEQ ID NO: 9074; SEQ ID NO: 9075; SEQ ID NO: 9076; SEQ ID NO: 9077; SEQ ID NO: 9078; SEQ ID NO: 9079; SEQ ID NO: 9080; SEQ ID NO: 9081; SEQ ID NO: 9082; SEQ ID NO: 9083; SEQ ID NO: 9084; SEQ ID NO: 9085; SEQ ID NO: 9086; SEQ ID NO: 9087; SEQ ID NO: 9088; SEQ ID NO: 9089; SEQ ID NO: 9090; SEQ ID NO: 9091; SEQ ID NO: 9092; SEQ ID NO: 9093; SEQ ID NO: 9094; SEQ ID NO: 9095; SEQ ID NO: 9096; SEQ ID NO: 9097; SEQ ID NO: 9098; SEQ ID NO: 9099; SEQ ID NO: 9100; SEQ ID NO: 9101; SEQ ID NO: 9102; SEQ ID NO: 9103; SEQ ID NO: 9104; SEQ ID NO: 9105; SEQ ID NO: 9106; SEQ ID NO: 9107; SEQ ID NO: 9108; SEQ ID NO: 9109; SEQ ID NO: 9110; SEQ ID NO: 9111; SEQ ID NO: 9112; SEQ ID NO: 9113; SEQ ID NO: 9114; SEQ ID NO: 9115; SEQ ID NO: 9116; SEQ ID NO: 9117; SEQ ID NO: 9118; SEQ ID NO: 9119; SEQ ID NO: 9120; SEQ ID NO: 9121; SEQ ID NO: 9122; SEQ ID NO: 9123; SEQ ID NO: 9124; SEQ ID NO: 9125; SEQ ID NO: 9126; SEQ ID NO: 9127; SEQ ID NO: 9128; SEQ ID NO: 9129; SEQ ID NO: 9130; SEQ ID NO: 9131; SEQ ID NO: 9132; SEQ ID NO: 9133; SEQ ID NO: 9134; SEQ ID NO: 9135; SEQ ID NO: 9136; SEQ ID NO: 9137; SEQ ID NO: 9138; SEQ ID NO: 9139; SEQ ID NO: 9140; SEQ ID NO: 9141; SEQ ID NO: 9142; SEQ ID NO: 9143; SEQ ID NO: 9144; SEQ ID NO: 9145; SEQ ID NO: 9146; SEQ ID NO: 9147; SEQ ID NO: 9148; SEQ ID NO: 9149; SEQ ID NO: 9150; SEQ ID NO: 9734; SEQ ID NO: 9735; SEQ ID NO: 9736 and SEQ ID NO: 9737.

In one embodiment, the H. pylori secreted polypeptide or a fragment thereof is a H. pylori periplasmic polypeptide or a fragment thereof having an amino acid sequence selected from the group consisting of SEQ ID NO: 8778; SEQ ID NO: 8779 and SEQ ID NO: 8780.

In a further embodiment, the H. pylori secreted polypeptide or a fragment thereof is a H. pylori polypeptide or a fragment thereof involved in secretion and adhesion having an amino acid sequence selected from the group consisting of SEQ ID NO: 8781; SEQ ID NO: 8782; SEQ ID NO: 8783; SEQ ID NO: 8784; SEQ ID NO: 8785; SEQ ID NO: 8786; SEQ ID NO: 8787; SEQ ID NO: 8788 and SEQ ID NO: 9734.

In a further embodiment, the H. pylori secreted polypeptide or a fragment thereof is a H. pylori chaperone polypeptide or a fragment thereof having an amino acid sequence selected from the group consisting of SEQ ID NO: 8789; SEQ ID NO: 8790; SEQ ID NO: 8791 and SEQ ID NO: 8792.

Particularly preferred is an isolated H. pylori secreted polypeptide or a fragment thereof, wherein the polypeptide is encoded by a nucleic acid sequence selected from the group consisting of SEQ ID NO: 4016; SEQ ID NO: 4017; SEQ ID NO: 4018; SEQ ID NO: 4019; SEQ ID NO: 4020; SEQ ID NO: 4021; SEQ ID NO: 4022; SEQ ID NO: 4023; SEQ ID NO: 4024; SEQ ID NO: 4025; SEQ ID NO: 4026; SEQ ID NO: 4027; SEQ ID NO: 4028; SEQ ID NO: 4029; SEQ ID NO: 4030; SEQ ID NO: 4031; SEQ ID NO: 4032; SEQ ID NO: 4033; SEQ ID NO: 4034; SEQ ID NO: 4035; SEQ ID NO: 4036; SEQ ID NO: 4037; SEQ ID NO: 4038; SEQ ID NO: 4039; SEQ ID NO: 4040; SEQ ID NO: 4041; SEQ ID NO: 4042; SEQ ID NO: 4043; SEQ ID NO: 4044; SEQ ID NO: 4045; SEQ ID NO: 4046; SEQ ID NO: 4047; SEQ ID NO: 4048; SEQ ID NO: 4049; SEQ ID NO: 4050; SEQ ID NO: 4051; SEQ ID NO: 4052; SEQ ID NO: 4053; SEQ ID NO: 4054; SEQ ID NO: 4055; SEQ ID NO: 4056; SEQ ID NO: 4057; SEQ ID NO: 4058; SEQ ID NO: 4059; SEQ ID NO: 4060; SEQ ID NO: 4061; SEQ ID NO: 4062; SEQ ID NO: 4063; SEQ ID NO: 4064; SEQ ID NO: 4065; SEQ ID NO: 4066; SEQ ID NO: 4067; SEQ ID NO: 4068; SEQ ID NO: 4069; SEQ ID NO: 4070; SEQ ID NO: 4071; SEQ ID NO: 4072; SEQ ID NO: 4073; SEQ ID NO: 4074; SEQ ID NO: 4075; SEQ ID NO: 4076; SEQ ID NO: 4077; SEQ ID NO: 4078; SEQ ID NO: 4079; SEQ ID NO: 4080; SEQ ID NO: 4081; SEQ ID NO: 4082; SEQ ID NO: 4083; SEQ ID NO: 4084; SEQ ID NO: 4085; SEQ ID NO: 4086; SEQ ID NO: 4087; SEQ ID NO: 4088; SEQ ID NO: 4089; SEQ ID NO: 4090; SEQ ID NO: 4091; SEQ ID NO: 4092; SEQ ID NO: 4093; SEQ ID NO: 4094; SEQ ID NO: 4095; SEQ ID NO: 4096; SEQ ID NO: 4097; SEQ ID NO: 4098; SEQ ID NO: 4099; SEQ ID NO: 4100; SEQ ID NO: 4101; SEQ ID NO: 4102; SEQ ID NO: 4103; SEQ ID NO: 4104; SEQ ID NO: 4105; SEQ ID NO: 4106; SEQ ID NO: 4107; SEQ ID NO: 4108; SEQ ID NO: 4109; SEQ ID NO: 4110; SEQ ID NO: 4111; SEQ ID NO: 4112; SEQ ID NO: 4113; SEQ ID NO: 4114; SEQ ID NO: 4115; SEQ ID NO: 4116; SEQ ID NO: 4117; SEQ ID NO: 4118; SEQ ID NO: 4119; SEQ ID NO: 4120; SEQ ID NO: 4121; SEQ ID NO: 4122; SEQ ID NO: 4123; SEQ ID NO: 4124; SEQ ID NO: 4125; SEQ ID NO: 4126; SEQ ID NO: 4127; SEQ ID NO: 4128; SEQ ID NO: 4129; SEQ ID NO: 4130; SEQ ID NO: 4131; SEQ ID NO: 4132; SEQ ID NO: 4133; SEQ ID NO: 4134; SEQ ID NO: 4135; SEQ ID NO: 4136; SEQ ID NO: 4137; SEQ ID NO: 4138; SEQ ID NO: 4139; SEQ ID NO: 4140; SEQ ID NO: 4141; SEQ ID NO: 4142; SEQ ID NO: 4143; SEQ ID NO: 4144; SEQ ID NO: 4145; SEQ ID NO: 4146; SEQ ID NO: 4147; SEQ ID NO: 4148; SEQ ID NO: 4149; SEQ ID NO: 4150; SEQ ID NO: 4151; SEQ ID NO: 4152; SEQ ID NO: 4153; SEQ ID NO: 4154; SEQ ID NO: 4155; SEQ ID NO: 4156; SEQ ID NO: 4157; SEQ ID NO: 4158; SEQ ID NO: 4159; SEQ ID NO: 4160; SEQ ID NO: 4161; SEQ ID NO: 4162; SEQ ID NO: 4163; SEQ ID NO: 4164; SEQ ID NO: 4165; SEQ ID NO: 4166; SEQ ID NO: 4167; SEQ ID NO: 4168; SEQ ID NO: 4169; SEQ ID NO: 4170; SEQ ID NO: 4171; SEQ ID NO: 4172; SEQ ID NO: 4173; SEQ ID NO: 4174; SEQ ID NO: 4175; SEQ ID NO: 4176; SEQ ID NO: 4177; SEQ ID NO: 4178; SEQ ID NO: 4179; SEQ ID NO: 4180; SEQ ID NO: 4181; SEQ ID NO: 4182; SEQ ID NO: 4183; SEQ ID NO: 4184; SEQ ID NO: 4185; SEQ ID NO: 4186; SEQ ID NO: 4187; SEQ ID NO: 4188; SEQ ID NO: 4189; SEQ ID NO: 4190; SEQ ID NO: 4191; SEQ ID NO: 4192; SEQ ID NO: 4193; SEQ ID NO: 4194; SEQ ID NO: 4195; SEQ ID NO: 4196; SEQ ID NO: 4197; SEQ ID NO: 4198; SEQ ID NO: 4199; SEQ ID NO: 4200; SEQ ID NO: 4201; SEQ ID NO: 4202; SEQ ID NO: 4203; SEQ ID NO: 4204; SEQ ID NO: 4205; SEQ ID NO: 4206; SEQ ID NO: 4207; SEQ ID NO: 4208; SEQ ID NO: 4209; SEQ ID NO: 4210; SEQ ID NO: 4211; SEQ ID NO: 4212; SEQ ID NO: 4213; SEQ ID NO: 4214; SEQ ID NO: 4215; SEQ ID NO: 4216; SEQ ID NO: 4217; SEQ ID NO: 4218; SEQ ID NO: 4219; SEQ ID NO: 4220; SEQ ID NO: 4221; SEQ ID NO: 4222; SEQ ID NO: 4223; SEQ ID NO: 4224; SEQ ID NO: 4225; SEQ ID NO: 4226; SEQ ID NO: 4227; SEQ ID NO: 4228; SEQ ID NO: 4229; SEQ ID NO: 4230; SEQ ID NO: 4231; SEQ ID NO: 4232; SEQ ID NO: 4233; SEQ ID NO: 4234; SEQ ID NO: 4235; SEQ ID NO: 4236; SEQ ID NO: 4237; SEQ ID NO: 4238; SEQ ID NO: 4239; SEQ ID NO: 4240; SEQ ID NO: 4241; SEQ ID NO: 4242; SEQ ID NO: 4243; SEQ ID NO: 4244; SEQ ID NO: 4245; SEQ ID NO: 4246; SEQ ID NO: 4247; SEQ ID NO: 4248; SEQ ID NO: 4249; SEQ ID NO: 4250; SEQ ID NO: 4251; SEQ ID NO: 4252; SEQ ID NO: 4253; SEQ ID NO: 4254; SEQ ID NO: 4255; SEQ ID NO: 4256; SEQ ID NO: 4257; SEQ ID NO: 4258; SEQ ID NO: 4259; SEQ ID NO: 4260; SEQ ID NO: 4261; SEQ ID NO: 4262; SEQ ID NO: 4263; SEQ ID NO: 4264; SEQ ID NO: 4265; SEQ ID NO: 4266; SEQ ID NO: 4267; SEQ ID NO: 4268; SEQ ID NO: 4269; SEQ ID NO: 4270; SEQ ID NO: 4271; SEQ ID NO: 4272; SEQ ID NO: 4273; SEQ ID NO: 4274; SEQ ID NO: 4275; SEQ ID NO: 4276; SEQ ID NO: 4277; SEQ ID NO: 4278; SEQ ID NO: 4279; SEQ ID NO: 4280; SEQ ID NO: 4281; SEQ ID NO: 4282; SEQ ID NO: 4283; SEQ ID NO: 4284; SEQ ID NO: 4285; SEQ ID NO: 4286; SEQ ID NO: 4287; SEQ ID NO: 4288; SEQ ID NO: 4289; SEQ ID NO: 4290; SEQ ID NO: 4291; SEQ ID NO: 4292; SEQ ID NO: 4293; SEQ ID NO: 4294; SEQ ID NO: 4295; SEQ ID NO: 4296; SEQ ID NO: 4297; SEQ ID NO: 4298; SEQ ID NO: 4299; SEQ ID NO: 4300; SEQ ID NO: 4301; SEQ ID NO: 4302; SEQ ID NO: 4303; SEQ ID NO: 4304; SEQ ID NO: 4305; SEQ ID NO: 4306; SEQ ID NO: 4307; SEQ ID NO: 4308; SEQ ID NO: 4309; SEQ ID NO: 4310; SEQ ID NO: 4311; SEQ ID NO: 4312; SEQ ID NO: 4313; SEQ ID NO: 4314; SEQ ID NO: 4315; SEQ ID NO: 4316; SEQ ID NO: 4317; SEQ ID NO: 4318; SEQ ID NO: 4319; SEQ ID NO: 4320; SEQ ID NO: 4321; SEQ ID NO: 4322; SEQ ID NO: 4323; SEQ ID NO: 4324; SEQ ID NO: 4325; SEQ ID NO: 4326; SEQ ID NO: 4327; SEQ ID NO: 4328; SEQ ID NO: 4329; SEQ ID NO: 4330; SEQ ID NO: 4331; SEQ ID NO: 4332; SEQ ID NO: 4333; SEQ ID NO: 4334; SEQ ID NO: 4335; SEQ ID NO: 4336; SEQ ID NO: 4337; SEQ ID NO: 4388; SEQ ID NO: 9622; SEQ ID NO: 9623; SEQ ID NO: 9624 and SEQ ID NO: 9625.

In one embodiment, the H. pylori secreted polypeptide or a fragment thereof is a H. pylori periplasmic polypeptide or a fragment thereof encoded by a nucleic acid having a nucleotide sequence selected from the group consisting of SEQ ID NO: 4016; SEQ ID NO: 4017 and SEQ ID NO: 4018.

In a further embodiment, the H. pylori secreted polypeptide or a fragment thereof is a H. pylori polypeptide or a fragment thereof involved in secretion and adhesion encoded by a nucleic acid sequence selected from the group consisting of SEQ ID NO: 4019; SEQ ID NO: 4020; SEQ ID NO: 4021; SEQ ID NO: 4022; SEQ ID NO: 4023; SEQ ID NO: 4024; SEQ ID NO: 4025; SEQ ID NO: 4026 and SEQ ID NO: 9622.

In a further embodiment, the H. pylori secreted polypeptide or a fragment thereof is a H. pylori chaperone polypeptide or a fragment thereof encoded by a nucleic acid having a nucleotide sequence selected from the group consisting of SEQ ID NO: 4027; SEQ ID NO: 4028; SEQ ID NO: 4029 and SEQ ID NO: 4030.

Particularly preferred is an isolated H. pylori cellular polypeptide or a fragment thereof, wherein the polypeptide has an amino acid sequence selected from the group consisting of SEQ ID NO: 9151; SEQ ID NO: 9152; SEQ ID NO: 9153; SEQ ID NO: 9154; SEQ ID NO: 9155; SEQ ID NO: 9156; SEQ ID NO: 9157; SEQ ID NO: 9158; SEQ ID NO: 9159; SEQ ID NO: 9160; SEQ ID NO: 9161; SEQ ID NO: 9162; SEQ ID NO: 9163; SEQ ID NO: 9164; SEQ ID NO: 9165; SEQ ID NO: 9166; SEQ ID NO: 9167; SEQ ID NO: 9168; SEQ ID NO: 9169; SEQ ID NO: 9170; SEQ ID NO: 9171; SEQ ID NO: 9172; SEQ ID NO: 9173; SEQ ID NO: 9174; SEQ ID NO: 9175; SEQ ID NO: 9176; SEQ ID NO: 9177; SEQ-ID NO: 9178; SEQ ID NO: 9179; SEQ ID NO: 9180; SEQ ID NO: 9181; SEQ ID NO: 9182; SEQ ID NO: 9183; SEQ ID NO: 9184; SEQ ID NO: 9185; SEQ ID NO: 9186; SEQ ID NO: 9187; SEQ ID NO: 9188; SEQ ID NO: 9189; SEQ ID NO: 9190; SEQ ID NO: 9191; SEQ ID NO: 9192; SEQ ID NO: 9193; SEQ ID NO: 9194; SEQ ID NO: 9195; SEQ ID NO: 9196; SEQ ID NO: 9197; SEQ ID NO: 9198; SEQ ID NO: 9199; SEQ ID NO: 9200; SEQ ID NO: 9201; SEQ ID NO: 9202; SEQ ID NO: 9203; SEQ ID NO: 9204; SEQ ID NO: 9205; SEQ ID NO: 9206; SEQ ID NO: 9207; SEQ ID NO: 9208; SEQ ID NO: 9209; SEQ ID NO: 9210; SEQ ID NO: 9211; SEQ ID NO: 9212; SEQ ID NO: 9213; SEQ ID NO: 9214; SEQ ID NO: 9215; SEQ ID NO: 9216; SEQ ID NO: 9217; SEQ ID NO: 9218; SEQ ID NO: 9219; SEQ ID NO: 9220; SEQ ID NO: 9221; SEQ ID NO: 9222; SEQ ID NO: 9223; SEQ ID NO: 9224; SEQ ID NO: 9225; SEQ ID NO: 9226; SEQ ID NO: 9227; SEQ ID NO: 9228; SEQ ID NO: 9229; SEQ ID NO: 9230; SEQ ID NO: 9231; SEQ ID NO: 9232; SEQ ID NO: 9233; SEQ ID NO: 9234; SEQ ID NO: 9235; SEQ ID NO: 9236; SEQ ID NO: 9237; SEQ ID NO: 9238; SEQ ID NO: 9239; SEQ ID NO: 9240; SEQ ID NO: 9241; SEQ ID NO: 9242; SEQ ID NO: 9243; SEQ ID NO: 9244; SEQ ID NO: 9245; SEQ ID NO: 9246; SEQ ID NO: 9247; SEQ ID NO: 9248; SEQ ID NO: 9249; SEQ ID NO: 9250; SEQ ID NO: 9251; SEQ ID NO: 9252; SEQ ID NO:9253; SEQ ID NO: 9254; SEQ ID NO: 9255; SEQ ID NO: 9256; SEQ ID NO: 9257; SEQ ID NO: 9258; SEQ ID NO: 9259; SEQ ID NO: 9260; SEQ ID NO: 9261; SEQ ID NO: 9262; SEQ ID NO: 9263; SEQ ID NO: 9264; SEQ ID NO: 9265; SEQ ID NO: 9266; SEQ ID NO: 9267; SEQ ID NO: 9268; SEQ ID NO: 9269; SEQ ID NO: 9270; SEQ ID NO: 9271; SEQ ID NO: 9272; SEQ ID NO: 9273; SEQ ID NO: 9274; SEQ ID NO: 9275; SEQ ID NO: 9276; SEQ ID NO: 9277; SEQ ID NO: 9278; SEQ ID NO: 9279; SEQ ID NO: 9280; SEQ ID NO: 9281; SEQ ID NO: 9282; SEQ ID NO: 9283; SEQ ID NO: 9284; SEQ ID NO: 9285; SEQ ID NO: 9286; SEQ ID NO: 9287; SEQ ID NO: 9288; SEQ ID NO: 9289; SEQ ID NO: 9290; SEQ ID NO: 9291; SEQ ID NO: 9292; SEQ ID NO: 9293; SEQ ID NO: 9294; SEQ ID NO: 9295; SEQ ID NO: 9296; SEQ ID NO: 9297; SEQ ID NO: 9298; SEQ ID NO: 9299; SEQ ID NO: 9300; SEQ ID NO: 9301; SEQ ID NO: 9302; SEQ ID NO: 9303; SEQ ID NO: 9304; SEQ ID NO: 9305; SEQ ID NO: 9306; SEQ ID NO: 9307; SEQ ID NO: 9308; SEQ ID NO: 9309; SEQ ID NO: 9310; SEQ ID NO: 9311; SEQ ID NO: 9312; SEQ ID NO: 9313; SEQ ID NO: 9314; SEQ ID NO: 9315; SEQ ID NO: 9316; SEQ ID NO: 9317; SEQ ID NO: 9318; SEQ ID NO: 9319; SEQ ID NO: 9320; SEQ ID NO: 9321; SEQ ID NO: 9322; SEQ ID NO: 9323; SEQ ID NO: 9324; SEQ ID NO: 9325; SEQ ID NO: 9326; SEQ ID NO: 9327; SEQ ID NO: 9328; SEQ ID NO: 9329; SEQ ID NO: 9330; SEQ ID NO: 9331; SEQ ID NO: 9332; SEQ ID NO: 9333; SEQ ID NO: 9334; SEQ ID NO: 9335; SEQ ID NO: 9336; SEQ ID NO: 9337; SEQ ID NO: 9338; SEQ ID NO: 9339; SEQ ID NO: 9340; SEQ ID NO: 9341; SEQ ID NO: 9342; SEQ ID NO: 9343; SEQ ID NO: 9344; SEQ ID NO: 9345; SEQ ID NO: 9346; SEQ ID NO: 9347; SEQ ID NO: 9348; SEQ ID NO: 9349; SEQ ID NO: 9350; SEQ ID NO: 9351; SEQ ID NO: 9352; SEQ ID NO: 9353; SEQ ID NO: 9354; SEQ ID NO: 9355; SEQ ID NO: 9356; SEQ ID NO: 9357; SEQ ID NO: 9358; SEQ ID NO: 9359; SEQ ID NO: 9360; SEQ ID NO: 9361; SEQ ID NO: 9362; SEQ ID NO: 9363; SEQ ID NO: 9364; SEQ ID NO: 9365; SEQ ID NO: 9366; SEQ ID NO: 9367; SEQ ID NO: 9368; SEQ ID NO: 9369; SEQ ID NO: 9370; SEQ ID NO: 9371; SEQ ID NO: 9372; SEQ ID NO: 9373; SEQ ID NO: 9374; SEQ ID NO: 9375; SEQ ID NO: 9376; SEQ ID NO: 9377; SEQ ID NO: 9378; SEQ ID NO: 9379; SEQ ID NO: 9380; SEQ ID NO: 9381; SEQ ID NO: 9382; SEQ ID NO: 9383; SEQ ID NO: 9384; SEQ ID NO: 9385; SEQ ID NO: 9386; SEQ ID NO: 9387; SEQ ID NO: 9388; SEQ ID NO: 9389; SEQ ID NO: 9390; SEQ ID NO: 9391; SEQ ID NO: 9392; SEQ ID NO: 9393; SEQ ID NO: 9394; SEQ ID NO: 9395; SEQ ID NO: 9396; SEQ ID NO: 9397; SEQ ID NO: 9398; SEQ ID NO: 9399; SEQ ID NO: 9400; SEQ ID NO: 9401; SEQ ID NO: 9402; SEQ ID NO: 9403; SEQ ID NO: 9404; SEQ ID NO: 9405; SEQ ID NO: 9406; SEQ ID NO: 9407; SEQ ID NO: 9408; SEQ ID NO: 9409; SEQ ID NO: 9410; SEQ ID NO: 9411; SEQ ID NO: 9412; SEQ ID NO: 9413; SEQ ID NO: 9414; SEQ ID NO: 9415; SEQ ID NO: 9416; SEQ ID NO: 9417; SEQ ID NO: 9418; SEQ ID NO: 9419; SEQ ID NO: 9420; SEQ ID NO: 9421; SEQ ID NO: 9422; SEQ ID NO: 9423; SEQ ID NO: 9424; SEQ ID NO: 9425; SEQ ID NO: 9426; SEQ ID NO: 9427; SEQ ID NO: 9428; SEQ ID NO: 9429; SEQ ID NO: 9430; SEQ ID NO: 9431; SEQ ID NO: 9432; SEQ ID NO: 9433; SEQ ID NO: 9434; SEQ ID NO: 9435; SEQ ID NO: 9436; SEQ ID NO: 9437; SEQ ID NO: 9438; SEQ ID NO: 9439; SEQ ID NO: 9440; SEQ ID NO: 9441; SEQ ID NO: 9442; SEQ ID NO: 9443; SEQ ID NO: 9444; SEQ ID NO: 9445; SEQ ID NO: 9446; SEQ ID NO: 9447; SEQ ID NO: 9448; SEQ ID NO: 9449; SEQ ID NO: 9450; SEQ ID NO: 9451; SEQ ID NO: 9452; SEQ ID NO: 9453; SEQ ID NO: 9454; SEQ ID NO: 9455; SEQ ID NO: 9456; SEQ ID NO: 9457; SEQ ID NO: 9458; SEQ ID NO: 9459; SEQ ID NO: 9460; SEQ ID NO: 9461; SEQ ID NO: 9462; SEQ ID NO: 9463; SEQ ID NO: 9464; SEQ ID NO: 9465; SEQ ID NO: 9466; SEQ ID NO: 9467; SEQ ID NO: 9738; SEQ ID NO: 9739; SEQ ID NO: 9740; SEQ ID NO: 9741; SEQ ID NO: 9742; SEQ ID NO: 9743; SEQ ID NO: 9744; SEQ ID NO: 9745; SEQ ID NO: 9746; SEQ ID NO: 9747 and SEQ ID NO: 9748.

Particularly preferred is an isolated H. pylori cellular polypeptide or a fragment thereof, wherein the polypeptide is encoded by a nucleic acid having a nucleotide sequence selected from the group consisting of SEQ ID NO: 4389; SEQ ID NO: 4390; SEQ ID NO: 4391; SEQ ID NO: 4392; SEQ ID NO: 4393; SEQ ID NO: 4394; SEQ ID NO: 4395; SEQ ID NO: 4396; SEQ ID NO: 4397; SEQ ID NO: 4398; SEQ ID NO: 4399; SEQ ID NO: 4400; SEQ ID NO: 4401; SEQ ID NO: 4402; SEQ ID NO: 4403; SEQ. ID NO: 4404; SEQ ID NO: 4405; SEQ ID NO: 4406; SEQ ID NO: 4407; SEQ ID NO: 4408; SEQ ID NO: 4409; SEQ ID NO: 4410; SEQ ID NO: 4411; SEQ ID NO: 4412; SEQ ID NO:4413; SEQ ID NO:4414; SEQ ID NO:4415; SEQ ID NO:4416; SEQ ID NO: 4417; SEQ ID NO: 4418; SEQ ID NO: 4419; SEQ ID NO: 4420; SEQ ID NO: 4421; SEQ ID NO: 4422; SEQ ID NO: 4423; SEQ ID NO: 4424; SEQ ID NO: 4425; SEQ ID NO: 4426; SEQ ID NO: 4427; SEQ ID NO: 4428; SEQ ID NO: 4429; SEQ ID NO: 4430; SEQ ID NO: 4431; SEQ ID NO: 4432; SEQ ID NO: 4433; SEQ ID NO: 4434; SEQ ID NO: 4435; SEQ ID NO: 4436; SEQ ID NO: 4437; SEQ ID NO: 4438; SEQ ID NO: 4439; SEQ ID NO: 4440; SEQ ID NO: 4441; SEQ ID NO: 4442; SEQ ID NO: 4443; SEQ ID NO: 4444; SEQ ID NO: 4445; SEQ ID NO: 4446; SEQ ID NO: 4447; SEQ ID NO: 4448; SEQ ID NO: 4449; SEQ ID NO: 4450; SEQ ID NO: 4451; SEQ ID NO: 4452; SEQ ID NO: 4453; SEQ ID NO: 4454; SEQ ID NO: 4455; SEQ ID NO: 4456; SEQ ID NO: 4457; SEQ ID NO: 4458; SEQ ID NO: 4459; SEQ ID NO: 4460; SEQ ID NO: 4461; SEQ ID NO: 4462; SEQ ID NO: 4463; SEQ ID NO: 4464; SEQ ID NO: 4465; SEQ ID NO: 4466; SEQ ID NO: 4467; SEQ ID NO: 4468; SEQ ID NO: 4469; SEQ ID NO: 4470; SEQ ID NO: 4471; SEQ ID NO: 4472; SEQ ID NO: 4473; SEQ ID NO: 4474; SEQ ID NO: 4475; SEQ ID NO: 4476; SEQ ID NO: 4477; SEQ ID NO:4478; SEQ ID NO:4479; SEQ ID NO:4480; SEQ ID NO: 4481; SEQ ID NO: 4482; SEQ ID NO: 4483; SEQ ID NO: 4484; SEQ ID NO: 4485; SEQ ID NO: 4486; SEQ ID NO: 4487; SEQ ID NO: 4488; SEQ ID NO: 4489; SEQ ID NO: 4490; SEQ ID NO: 4491; SEQ ID NO: 4492; SEQ ID NO: 4493; SEQ ID NO: 4494; SEQ ID NO: 4495; SEQ ID NO: 4496; SEQ ID NO: 4497; SEQ ID NO: 4498; SEQ ID NO: 4499; SEQ ID NO: 4500; SEQ ID NO: 4501; SEQ ID NO: 4502; SEQ ID NO: 4503; SEQ ID NO: 4504; SEQ ID NO: 4505; SEQ ID NO: 4506; SEQ ID NO: 4507; SEQ ID NO: 4508; SEQ ID NO: 4509; SEQ ID NO: 4510; SEQ ID NO: 4511; SEQ ID NO: 4512; SEQ ID NO: 4513; SEQ ID NO: 4514; SEQ ID NO: 4515; SEQ ID NO: 4516; SEQ ID NO: 4517; SEQ ID NO: 4518; SEQ ID NO: 4519; SEQ ID NO: 4520; SEQ ID NO: 4521; SEQ ID NO: 4522; SEQ ID NO: 4523; SEQ ID NO: 4524; SEQ ID NO: 4525; SEQ ID NO: 4526; SEQ ID NO: 4527; SEQ ID NO: 4528; SEQ ID NO: 4529; SEQ ID NO: 4530; SEQ ID NO: 4531; SEQ ID NO: 4532; SEQ ID NO: 4533; SEQ ID NO: 4534; SEQ ID NO: 4535; SEQ ID NO: 4536; SEQ ID NO: 4537; SEQ ID NO: 4538; SEQ ID NO: 4539; SEQ ID NO: 4540; SEQ ID NO: 4541; SEQ ID NO: 4542; SEQ ID NO: 4543; SEQ ID NO: 4544; SEQ ID NO: 4545; SEQ ID NO: 4546; SEQ ID NO: 4547; SEQ ID NO: 4548; SEQ ID NO: 4549; SEQ ID NO: 4550; SEQ ID NO: 4551; SEQ ID NO: 4552; SEQ ID NO: 4553; SEQ ID NO: 4554; SEQ ID NO: 4555; SEQ ID NO: 4556; SEQ ID NO: 4557; SEQ ID NO: 4558; SEQ ID NO: 4559; SEQ ID NO: 4560; SEQ ID NO: 4561; SEQ ID NO: 4562; SEQ ID NO: 4563; SEQ ID NO: 4564; SEQ ID NO: 4565; SEQ ID NO: 4566; SEQ ID NO: 4567; SEQ ID NO: 4568; SEQ ID NO: 4569; SEQ ID NO: 4570; SEQ ID NO: 4571; SEQ ID NO: 4572; SEQ ID NO: 4573; SEQ ID NO: 4574; SEQ ID NO: 4575; SEQ ID NO: 4576; SEQ ID NO: 4577; SEQ ID NO: 4578; SEQ ID NO: 4579; SEQ ID NO: 4580; SEQ ID NO: 4581; SEQ ID NO: 4582; SEQ ID NO: 4583; SEQ ID NO: 4584; SEQ ID NO: 4585; SEQ ID NO: 4586; SEQ ID NO: 4587; SEQ ID NO: 4588; SEQ ID NO: 4589; SEQ ID NO: 4590; SEQ ID NO: 4591; SEQ ID NO: 4592; SEQ ID NO: 4593; SEQ ID NO: 4594; SEQ ID NO: 4595; SEQ ID NO: 4596; SEQ ID NO: 4597; SEQ ID NO: 4598; SEQ ID NO: 4599; SEQ ID NO: 4600; SEQ ID NO: 4601; SEQ ID NO: 4602; SEQ ID NO: 4603; SEQ ID NO: 4604; SEQ ID NO: 4605; SEQ ID NO: 4606; SEQ ID NO: 4607; SEQ ID NO: 4608; SEQ ID NO: 4609; SEQ ID NO: 4610; SEQ ID NO: 4611; SEQ ID NO: 4612; SEQ ID NO: 4613; SEQ ID NO: 4614; SEQ ID NO: 4615; SEQ ID NO: 4616; SEQ ID NO: 4617; SEQ ID NO: 4618; SEQ ID NO: 4619; SEQ ID NO: 4620; SEQ ID NO: 4621; SEQ ID NO: 4622; SEQ ID NO: 4623; SEQ ID NO: 4624; SEQ ID NO: 4625; SEQ ID NO: 4626; SEQ ID NO: 4627; SEQ ID NO: 4628; SEQ ID NO: 4629; SEQ ID NO: 4630; SEQ ID NO: 4631; SEQ ID NO: 4632; SEQ ID NO: 4633; SEQ ID NO: 4634; SEQ ID NO: 4635; SEQ ID NO: 4636; SEQ ID NO: 4637; SEQ ID NO: 4638; SEQ ID NO: 4639; SEQ ID NO: 4640; SEQ ID NO: 4641; SEQ ID NO: 4642; SEQ ID NO: 4643; SEQ ID NO: 4644; SEQ ID NO: 4645; SEQ ID NO: 4646; SEQ ID NO: 4647; SEQ ID NO: 4648; SEQ ID NO: 4649; SEQ ID NO: 4650; SEQ ID NO: 4651; SEQ ID NO: 4652; SEQ ID NO: 4653; SEQ ID NO: 4654; SEQ ID NO: 4655; SEQ ID NO: 4656; SEQ ID NO: 4657; SEQ ID NO: 4658; SEQ ID NO: 4659; SEQ ID NO: 4660; SEQ ID NO: 4661; SEQ ID NO: 4662; SEQ ID NO: 4663; SEQ ID NO: 4664; SEQ ID NO: 4665; SEQ ID NO: 4666; SEQ ID NO: 4667; SEQ ID NO: 4668; SEQ ID NO: 4669; SEQ ID NO: 4670; SEQ ID NO: 4671; SEQ ID NO: 4672; SEQ ID NO: 4673; SEQ ID NO: 4674; SEQ ID NO: 4675; SEQ ID NO: 4676; SEQ ID NO: 4677; SEQ ID NO: 4678; SEQ ID NO: 4679; SEQ ID NO: 4680; SEQ ID NO: 4681; SEQ ID NO: 4682; SEQ ID NO: 4683; SEQ ID NO: 4684; SEQ ID NO: 4685; SEQ ID NO: 4686; SEQ ID NO: 4687; SEQ ID NO: 4688; SEQ ID NO: 4689; SEQ ID NO: 4690; SEQ ID NO: 4691; SEQ ID NO: 4692; SEQ ID NO: 4693; SEQ ID NO: 4694; SEQ ID NO: 4695; SEQ ID NO: 4696; SEQ ID NO: 4697; SEQ ID NO: 4698; SEQ ID NO: 4699; SEQ ID NO: 4700; SEQ ID NO: 4701; SEQ ID NO: 4702; SEQ ID NO: 4703; SEQ ID NO: 4704; SEQ ID NO: 4705; SEQ ID NO: 9626; SEQ ID NO: 9627; SEQ ID NO: 9628; SEQ ID NO: 9629; SEQ ID NO: 9630; SEQ ID NO: 9631; SEQ ID NO: 9632; SEQ ID NO: 9633; SEQ ID NO: 9634; SEQ ID NO: 9635 and SEQ ID NO: 9636.

Particularly preferred is an isolated H. pylori membrane associated polypeptide or a fragment thereof, wherein the polypeptide has an amino acid sequence selected from the group consisting of SEQ ID NO: 9468; SEQ ID NO: 9469; SEQ ID NO: 9470; SEQ ID NO: 9471; SEQ ID NO: 9472; SEQ ID NO: 9473; SEQ ID NO: 9474; SEQ ID NO: 9475; SEQ ID NO: 9476; SEQ ID NO: 9477; SEQ ID NO: 9478; SEQ ID NO: 9479; SEQ ID NO: 9480; SEQ ID NO: 9481; SEQ ID NO: 9482; SEQ ID NO: 9483; SEQ ID NO: 9484; SEQ ID NO: 9485; SEQ ID NO: 9486; SEQ ID NO: 9487; SEQ ID NO: 9488; SEQ ID NO: 9489; SEQ ID NO: 9490; SEQ ID NO: 9491; SEQ ID NO: 9492; SEQ ID NO: 9493; SEQ ID NO: 9494; SEQ ID NO: 9495; SEQ ID NO: 9496; SEQ ID NO: 9497; SEQ ID NO: 9498; SEQ ID NO: 9499; SEQ ID NO: 9500; SEQ ID NO: 9501; SEQ ID NO: 9502; SEQ ID NO: 9503; SEQ ID NO: 9504; SEQ ID NO: 9505; SEQ ID NO: 9506; SEQ ID NO: 9507; SEQ ID NO: 9508; SEQ ID NO: 9509; SEQ ID NO: 9510; SEQ ID NO: 9511; SEQ ID NO: 9512; SEQ ID NO: 9513; SEQ ID NO: 9514; SEQ ID NO: 9515; SEQ ID NO: 9516; SEQ ID NO: 9517; SEQ ID NO: 9518; SEQ ID NO: 9519; SEQ ID NO: 9520; SEQ ID NO: 9521; SEQ ID NO: 9522; SEQ ID NO: 9523 and SEQ ID NO: 9524.

In another aspect, the invention features a chimeric H. pylori polypeptide comprising at least two H. pylori polypeptides or fragments thereof, wherein the polypeptides are encoded by nucleic acid sequences selected from the group consisting of SEQ ID NO:1-SEQ ID NO:4762 and SEQ ID NO: 9525-SEQ ID NO: 9636.

In another aspect, the invention features a chimeric H. pylori polypeptide comprising at least two H. pylori polypeptides or fragments thereof, wherein the polypeptides are selected from the group consisting of SEQ ID NO: 4763-SEQ ID NO: 9524 and SEQ ID NO: 9637-SEQ ID NO: 9748.

In another aspect, the invention features a fusion protein comprising an H. pylori polypeptide which comprises an amino acid sequence selected from the group consisting of SEQ ID NO: 4763-SEQ ID NO: 9524 and SEQ ID NO: 9637-SEQ ID NO: 9748 operatively linked to a non-H. pylori polypeptide.

In another aspect, the invention features a vaccine formulation for prophylactic or therapeutic treatment of an H. pylori infection comprising an effective amount of at least one isolated nucleic acid of the invention.

Preferably, the invention features a vaccine formulation for prophylactic or therapeutic treatment of an H. pylori infection comprising an effective amount of at least one isolated nucleic acid encoding an H. pylori outer membrane polypeptide or a fragment thereof, which nucleic acid is selected from the group consisting of SEQ ID NO: 212, SEQ ID NO: 254, SEQ ID NO: 205, SEQ ID NO: 329, SEQ ID NO: 384, SEQ ID NO: 377 and SEQ ID NO: 289.

Preferably, the invention features a vaccine formulation for prophylactic or therapeutic treatment of an H. pylori infection comprising an effective amount of at least one isolated nucleic acid encoding an H. pylori cell envelope polypeptide or a fragment thereof, which nucleic acid is selected from the group consisting of SEQ ID NO: 469, SEQ ID NO: 89, SEQ ID NO: 4286, SEQ ID NO: 419, SEQ ID NO: 9618 and SEQ ID NO: 3253.

In another aspect, the invention features a vaccine formulation for prophylactic or therapeutic treatment of an H. pylori infection comprising an effective amount of at least one H. pylori polypeptide of the invention.

Preferably, the invention features a vaccine formulation for prophylactic or therapeutic treatment of an H. pylori infection comprising an effective amount of at least one H. pylori outer membrane polypeptide or a fragment thereof, which polypeptide is selected from the group consisting of SEQ ID NO: 4974, SEQ ID NO: 5016, SEQ ID NO: 4967, SEQ ID NO: 5091, SEQ ID NO: 5146, SEQ ID NO: 5139 and SEQ ID NO: 5051.

Preferably, the invention features a vaccine formulation for prophylactic or therapeutic treatment of an H. pylori infection comprising an effective amount of at least one H. pylori cell envelope polypeptide or a fragment thereof, which polypeptide is selected from the group consisting of SEQ ID NO: 5231, SEQ ID NO: 4851, SEQ ID NO: 9048, SEQ ID NO: 5181, SEQ ID NO: 9730 and SEQ ID NO: 8015.

Preferably, the vaccine formulation of the invention further includes a pharmaceutically acceptable carrier. In one embodiment, the pharmaceutically acceptable carrier includes an adjuvant. In another embodiment, the pharmaceutically acceptable carrier includes a delivery system, e.g., a live vector, e.g., a bacteria or a virus. In another embodiment, the pharmaceutically acceptable carrier includes both an adjuvant and a delivery system.

In another aspect, the invention features a method of treating or reducing a risk of H. pylori infection in a subject. The method includes administering to a subject a vaccine formulation of the invention, such that treatment or reduction of risk of H. pylori infection occurs.

In another aspect, the invention features a method of producing a vaccine formulation of the invention. The method includes combining at least one isolated H. pylori polypeptide or a fragment thereof selected from the group consisting of SEQ ID NO: 4763-SEQ ID NO: 9524 and SEQ ID NO: 9637-SEQ ID NO: 9748 with a pharmaceutically acceptable carrier to thereby form a vaccine formulation.

In another aspect, the invention features a method of producing a vaccine formulation of the invention. The method includes culturing a cell under conditions that permit expression of an H. pylori polypeptide or a fragment thereof selected from the group consisting of SEQ ID NO: 4763-SEQ ID NO: 9524 and SEQ ID NO: 9637-SEQ ID NO: 9748 isolating the H. pylori polypeptide from the cell; and combining at least one isolated H. pylori polypeptide or a fragment thereof with a pharmaceutically acceptable carrier to thereby form a vaccine formulation.

In another aspect, the invention pertains to any individual H. pylori polypeptide member or nucleic acid encoding such a member from the above-identified groups of H. pylori polypeptides.

In another aspect, the invention features nucleic acids capable of binding mRNA of H. pylori. Such nucleic acid is capable of acting as antisense nucleic acid to control the translation of mRNA of H. pylori. A further aspect features a nucleic acid which is capable of binding specifically to an H. pylori nucleic acid. These nucleic acids are also referred to herein as complements and have utility as probes and as capture reagents.

In another aspect, the invention features an expression system comprising an open reading frame corresponding to H. pylori nucleic acid. The nucleic acid further comprises a control sequence compatible with an intended host. The expression system is useful for making polypeptides corresponding to H. pylori nucleic acid.

In another aspect, the invention features a cell transformed with the expression system to produce H. pylori polypeptides.

In another aspect, the invention features a method of generating antibodies against H. pylori polypeptides which are capable of binding specifically to H. pylori polypeptides. Such antibodies have utility as reagents for immunoassays to evaluate the abundance and distribution of H. pylori-specific antigens.

In another aspect, the invention features a method of generating vaccines for immunizing an individual against H. pylori. The vaccination method includes: immunizing a subject with at least one H. pylori polypeptide according to the present invention, e.g., a surface or secreted polypeptide, or active portion thereof, and a pharmaceutically acceptable carrier. Such vaccines have therapeutic and/or prophylactic utilities.

In another aspect, the invention provides a method for generating a vaccine comprising a modified immunogenic H. pylori polypeptide, e.g., a surface or secreted polypeptide, or active portion thereof, and a pharmacologically acceptable carrier.

In another aspect, the invention features a method of evaluating a compound, e.g. a polypeptide, e.g., a fragment of a host cell polypeptide, for the ability to bind an H. pylori polypeptide. The method includes: contacting the candidate compound with an H. pylori polypeptide and determining if the compound binds or otherwise interacts with an H. pylori polypeptide. Compounds which bind H. pylori are candidates as activators or inhibitors of the bacterial life cycle. These assays can be performed in vitro or in vivo.

In another aspect, the invention features a method of evaluating a compound, e.g. a polypeptide, e.g., a fragment of a host cell polypeptide, for the ability to bind an H. pylori nucleic acid, e.g., DNA or RNA. The method includes: contacting the candidate compound with an H. pylori nucleic acid and determining if the compound binds or otherwise interacts with an H. pylori polypeptide. Compounds which bind H. pylori are candidates as activators or inhibitors of the bacterial life cycle. These assays can be performed in vitro or in vivo.

The invention features H. pylori polypeptides, preferably a substantially pure preparation of an H. pylori polypeptide, or a recombinant H. pylori polypeptide. In preferred embodiments: the polypeptide has biological activity; the polypeptide has an amino acid sequence at least 60%, 70%, 80%, 90%, 95%, 98%, or 99% identical or homologous to an amino acid sequence of the invention contained in the Sequence Listing, preferably it has about 65% sequence identity with an amino acid sequence of the invention contained in the Sequence Listing, and most preferably it has about 92% to about 99% sequence identity with an amino acid sequence of the invention contained in the Sequence Listing; the polypeptide has an amino acid sequence essentially the same as an amino acid sequence of the invention contained in the Sequence Listing; the polypeptide is at least 5, 10, 20, 50, 100, or 150 amino acid residues in length; the polypeptide includes at least 5, preferably at least 10, more preferably at least 20, more preferably at least 50, 100, or 150 contiguous amino acid residues of the invention contained in the Sequence Listing. In yet another preferred embodiment, the amino acid sequence which differs in sequence identity by about 7% to about 8% from the H. pylori amino acid sequences of the invention contained in the Sequence Listing is also encompassed by the invention.

In preferred embodiments: the H. pylori polypeptide is encoded by a nucleic acid of the invention contained in the Sequence Listing, or by a nucleic acid having at least 60%, 70%, 80%, 90%, 95%, 98%, or 99% homology with a nucleic acid of the invention contained in the Sequence Listing.

In a preferred embodiment, the subject H. pylori polypeptide differs in amino acid sequence at 1, 2, 3, 5, 10 or more residues from a sequence of the invention contained in the Sequence Listing. The differences, however, are such that the H. pylori polypeptide exhibits an H. pylori biological activity, e.g., the H. pylori polypeptide retains a biological activity of a naturally occurring H. pylori polypeptide.

In preferred embodiments, the polypeptide includes all or a fragment of an amino acid sequence of the invention contained in the Sequence Listing; fused, in reading frame, to additional amino acid residues, preferably to residues encoded by genomic DNA 5′ or 3′ to the genomic DNA which encodes a sequence of the invention contained in the Sequence Listing.

In yet other preferred embodiments, the H. pylori polypeptide is a recombinant fusion protein having a first H. pylori polypeptide portion and a second polypeptide portion, e.g., a second polypeptide portion having an amino acid sequence unrelated to H. pylori. The second polypeptide portion can be, e.g., any of glutathione-S-transferase, a DNA binding domain, or a polymerase activating domain. In preferred embodiment the fusion protein can be used in a two-hybrid assay.

Polypeptides of the invention include those which arise as a result of alternative transcription events, alternative RNA splicing events, and alternative translational and postranslational events.

The invention also encompasses an immunogenic component which includes at least one H. pylori polypeptide in an immunogenic preparation; the immunogenic component being capable of eliciting an immune response specific for the H. pylori polypeptide, e.g., a humoral response, an antibody response, or a cellular response. In preferred embodiments, the immunogenic component comprises at least one antigenic determinant from a polypeptide of the invention contained in the Sequence Listing.

In another aspect, the invention provides a substantially pure nucleic acid having a nucleotide sequence which encodes an H. pylori polypeptide. In preferred embodiments: the encoded polypeptide has biological activity; the encoded polypeptide has an amino acid sequence at least 60%, 70%, 80%, 90%, 95%, 98%, or 99% homologous to an amino acid sequence of the invention contained in the Sequence Listing; the encoded polypeptide has an amino acid sequence essentially the same as an amino acid sequence of the invention contained in the Sequence Listing; the encoded polypeptide is at least 5, 10, 20, 50, 100, or 150 amino acids in length; the encoded polypeptide comprises at least 5, preferably at least 10, more preferably at least 20, more preferably at least 50, 100, or 150 contiguous amino acids of the invention contained in the Sequence Listing.

In preferred embodiments: the nucleic acid of the invention is that contained in the Sequence Listing; the nucleic acid is at least 60%, 70%, 80%, 90%, 96%, 98%, or 99% homologous with a nucleic acid sequence of the invention contained in the Sequence Listing.

In a preferred embodiment, the encoded H. pylori polypeptide differs (e.g., by amino acid substitution, addition or deletion of at least one amino acid residue) in amino acid sequence at 1, 2, 3, 5, 10 or more residues, from a sequence of the invention contained in the Sequence Listing. The differences, however, are such that: the H. pylori encoded polypeptide exhibits a H. pylori biological activity, e.g., the encoded H. pylori enzyme retains a biological activity of a naturally occurring H. pylori.

In preferred embodiments, the encoded polypeptide includes all or a fragment of an amino acid sequence of the invention contained in the Sequence Listing; fused, in reading frame, to additional amino acid residues, preferably to residues encoded by genomic DNA 5′ or 3′ to the genomic DNA which encodes a sequence of the invention contained in the Sequence Listing.

In preferred embodiments, the subject H. pylori nucleic acid will include a transcriptional regulatory sequence, e.g. at least one of a transcriptional promoter or transcriptional enhancer sequence, operably linked to the H. pylori gene sequence, e.g., to render the H. pylori gene sequence suitable for expression in a recombinant host cell.

In yet a further preferred embodiment, the nucleic acid which encodes an H. pylori polypeptide of the invention, hybridizes under stringent conditions to a nucleic acid probe corresponding to at least 8 consecutive nucleotides of the invention contained in the Sequence Listing; more preferably to at least 12 consecutive nucleotides of the invention contained in the Sequence Listing; more preferably to at least 20 consecutive nucleotides of the invention contained in the Sequence Listing; more preferably to at least 40 consecutive nucleotides of the invention contained in the Sequence Listing.

In a preferred embodiment, the nucleic acid encodes a peptide which differs by at least one amino acid residue from the sequences of the invention contained in the Sequence Listing.

In a preferred embodiment, the nucleic acid differs by at least one nucleotide from a nucleotide sequence of the invention contained in the Sequence Listing which encodes amino acids of the invention contained in the Sequence Listing.

In another aspect, the invention encompasses: a vector including a nucleic acid which encodes an H. pylori polypeptide or an H. pylori polypeptide variant as described herein; a host cell transfected with the vector; and a method of producing a recombinant H. pylori polypeptide or H. pylori polypeptide variant; including culturing the cell, e.g., in a cell culture medium, and isolating the H. pylori or H. pylori polypeptide variant, e.g., from the cell or from the cell culture medium.

In another aspect, the invention features, a purified recombinant nucleic acid having at least 50%, 60%, 70%, 80%, 90%, 95%, 98%, or 99% homology with a sequence of the invention contained in the Sequence Listing.

The invention also provides a probe or primer which includes a substantially purified oligonucleotide. The oligonucleotide includes a region of nucleotide sequence which hybridizes under stringent conditions to at least 8 consecutive nucleotides of sense or antisense sequence of the invention contained in the Sequence Listing, or naturally occurring mutants thereof. In preferred embodiments, the probe or primer further includes a label group attached thereto. The label group can be, e.g., a radioisotope, a fluorescent compound, an enzyme, and/or an enzyme co-factor. Preferably the oligonucleotide is at least 8 and less than 10, 20, 30, 50, 100, or 150 nucleotides in length.

The invention also provides an isolated H. pylori polypeptide which is encoded by a nucleic acid which hybridizes under stringent hybridization conditions to a nucleic acid contained in the Sequence Listing.

The invention further provides nucleic acids, e.g., RNA or DNA, encoding a polypeptide of the invention. This includes double stranded nucleic acids as well as coding and antisense single strands.

The H. pylori strain, from which genomic sequences have been sequenced, has been deposited in the American Type Culture Collection (ATCC # 55679; deposited by Genome Therapeutics Corporation, 100 Beaver Street, Waltham, Mass. 02154) as strain HP-J99.

Included in the invention are: allelic variations; natural mutants; induced mutants; proteins encoded by DNA that hybridizes under high or low stringency conditions to a nucleic acid which encodes a polypeptide of the invention contained in the Sequence Listing (for definitions of high and low stringency see Current Protocols in Molecular Biology, John Wiley & Sons, New York, 1989, 6.3.1-6.3.6 and 6.4.1-6.4.10, hereby incorporated by reference); and, polypeptides specifically bound by antisera to H. pylori polypeptides, especially by antisera to an active site or binding domain of H. pylori polypeptide. The invention also includes fragments, preferably biologically active fragments. These and other polypeptides are also referred to herein as H. pylori polypeptide analogs or variants.

Putative functions have been determined for several of the H. pylori polypeptides of the invention, as shown in Table 1.

Accordingly, uses of the claimed H. pylori polypeptides based on these identified functions, as well as other functions as described herein, are also within the scope of the invention.

In addition, the present invention encompasses H. pylori polypeptides characterized as shown in Table 1 below, including: H. pylori cell envelope proteins, H. pylori secreted proteins, H. pylori cytoplasmic proteins and H. pylori cellular proteins. Members of these groups were identified by BLAST homology searches and by searches for secretion signal or transmembrane protein motifs. Polypeptides related by significant homology to the polypeptides of Table 1 are also considered to be classified in the manner of the homologs shown in Table 1.

TABLE 1

| Previously-Filed ORF | ntSeqID | aaSeqID |
|---|---|---|
| A. CELL ENVELOPE PROTEINS | | |
| A.1 Flagella-associated proteins | | |
| hp6e12267_29298130_c2_52 | 1 | 4763 |
| 29298130 | 2 | 4764 |
| 12ge20305orf11 | 3 | 4765 |
| 01ce11016orf18 | 4 | 4766 |
| hp6p80503_34199050_f1_3 | 5 | 4767 |
| hp6p80503_3986693_f2_10 | 6 | 4768 |
| 01ce11016orf21 | 7 | 4769 |
| hp6p80503_58562_f2_9 | 8 | 4770 |
| hpy01ce11016orf_0013.aa | 9 | 4771 |
| 01ce61016_58562_c1_111 | 10 | 4772 |
| 16219090 | 11 | 4773 |
| 05ep20322orf11 | 12 | 4774 |
| hpy05ep20322orf_0014.aa | 13 | 4775 |
| hp6p12311_4428127_f2_2 | 14 | 4776 |
| 02ge10116_26382032_c3_129 | 15 | 4777 |
| 29454837 | 16 | 4778 |
| 02ge10116_29454837_c2_27 | 17 | 4779 |
| 02ge20116orf34 | 18 | 4780 |
| 02ge10116_24322632_c3_128 | 19 | 4781 |
| 02ge10116_32679687_c1_73 | 20 | 4782 |
| 04gp11701_16984442_f3_17 | 21 | 4783 |
| 16984442 | 22 | 4784 |
| hp3e11168orf2 | 23 | 4785 |
| 22692187 | 24 | 4786 |
| hp5p15212orf1 | 25 | 4787 |
| 02ce10213orf7 | 26 | 4788 |
| hpy02ce10213orf_0001.aa | 27 | 4789 |
| hp5p15212_7289075_f2_5 | 28 | 4790 |
| 05ae11611orf1 | 29 | 4791 |
| hpy05ae11611orf_0001.aa | 30 | 4792 |
| 06ap30322_1986458_c3_31 | 31 | 4793 |
| 06ap10322_22663402_c2_31 | 32 | 4794 |
| 25525277 | 33 | 4795 |
| 06ep11202_25525277_c1_21 | 34 | 4796 |
| 06cp20302orf12 | 35 | 4797 |
| 203192 | 36 | 4798 |
| 06ep11202_6914712_c3_31 | 37 | 4799 |
| 06cp20302orf10 | 38 | 4800 |
| 1171928 | 39 | 4801 |
| 04cp11202_21699087_f1_9 | 40 | 4802 |
| 04ge11713orf5 | 41 | 4803 |
| hpy13ap11517orf_0014.aa | 42 | 4804 |
| 06gp71906_24417558_c2_160 | 43 | 4805 |
| 3942217 | 44 | 4806 |
| 06gp71906_3942217_c2_161 | 45 | 4807 |
| hp3e11122orf5 | 46 | 4808 |
| hpy11ap10314orf_0002.aa | 47 | 4809 |
| 11ap10314orf2 | 48 | 4810 |
| hpy11ap10314orf_0001.aa | 49 | 4811 |
| hp3e11122orf1 | 50 | 4812 |
| 06gp71906_4822162_c3_196 | 51 | 4813 |
| 14cp20718_24882763_f2_3 | 52 | 4814 |
| 24882763 | 53 | 4815 |
| 29zp10241orf6 | 54 | 4816 |
| 02cp20821orf3 | 55 | 4817 |
| hp5p15861_4556533_f2_6 | 56 | 4818 |
| 01ap11502orf1 | 57 | 4819 |
| 14ee41924_4022217_f3_49 | 58 | 4820 |
| 917152 | 59 | 4821 |
| 07ap11213_23470127_f3_16 | 60 | 4822 |
| hp2e10911orf5 | 61 | 4823 |
| hp4e13394_3368767_c1_80 | 62 | 4824 |
| 09ap11406orf2 | 63 | 4825 |
| hpy01ge10203orf_0004.aa | 64 | 4826 |
| hpy01ge10203orf_0002.aa | 65 | 4827 |
| hpy01ge10203orf_0001.aa | 66 | 4828 |
| 01ge10203orf2 | 67 | 4829 |
| hp1p13939_24322162_f3_17 | 68 | 4830 |
| 03ae10516orf14 | 69 | 4831 |
| 14gp12015orf6 | 70 | 4832 |
| 14gp12015orf8 | 71 | 4833 |
| 13727311 | 72 | 4834 |
| 06ep30223_13727311_c3_159 | 73 | 4835 |
| hpy02ae11612orf_0023.aa | 74 | 4836 |
| 01ce21104_4961067_c1_61 | 75 | 4837 |
| 01gp10401orf1 | 76 | 4838 |
| 26588588 | 77 | 4839 |
| hp6p10590_42501_c2_32 | 78 | 4840 |
| 01gp10401orf5 | 79 | 4841 |
| 01gp10401orf4 | 80 | 4842 |
| hpy01gp10401orf_0001.aa | 81 | 4843 |
| hp6p10590_4567317_c1_26 | 82 | 4844 |
| 12ap11614_24415965_f3_5 | 83 | 4845 |
| 12ap11614orf5 | 84 | 4846 |
| 19557055 | 85 | 4847 |
| hp7e10429_24423462_c3_47 | 86 | 4848 |
| 07ge20415orf27 | 87 | 4849 |
| 07ge20415orf28 | 88 | 4850 |
| 26380318 | 89 | 4851 |
| 07ge20415orf34 | 90 | 4852 |
| hp7e10429_4881342_c1_31 | 91 | 4853 |
| 07ge31107orf5 | 92 | 4854 |
| 07ge31107orf1 | 93 | 4855 |
| hpy07ge31107orf_0001.aa | 94 | 4856 |
| 07ge31107orf3 | 95 | 4857 |
| 07ge31107orf2 | 96 | 4858 |
| hp6e50324_32692588_c2_11 | 97 | 4859 |
| hpy02ae31112orf_0001.aa | 98 | 4860 |
| 11ap20714_2165628_c2_83 | 99 | 4861 |
| 14ee11217orf3 | 100 | 4862 |
| hpy14ee11217orf_0001.aa | 101 | 4863 |
| 11ap20714_5271967_c1_60 | 102 | 4864 |
| hp6p80503_13673962_f1_2 | 103 | 4865 |
| 21699087 | 9525 | 9637 |
| 25478375 | 9526 | 9638 |
| 36111066 | 9527 | 9639 |
| A.2 Outer membrane | | |
| A.2.1 Terminal phe residue | | |
| 09cp61003_14562637_c2_93 | 104 | 4866 |
| 12ge10305orf13 | 105 | 4867 |
| 03gp20123orf3 | 106 | 4868 |
| 12ge10305orf9 | 107 | 4869 |
| hp6e12267_12931513_f1_11 | 108 | 4870 |
| 01ce61016_12931513_c2_106 | 109 | 4871 |
| 11ee10423orf2 | 110 | 4872 |
| 11ee10423orf1 | 111 | 4873 |
| 09cp61003_24063587_c1_74 | 112 | 4874 |
| 21503772 | 113 | 4875 |
| 30478562 | 114 | 4876 |
| 12ge10305orf10 | 115 | 4877 |
| 12ge10305orf1 | 116 | 4878 |
| 09cp61003_30478562_c3_106 | 117 | 4879 |
| hp6e12267_30478562_f3_33 | 118 | 4880 |
| 1431462 | 119 | 4881 |
| 09cp11003_5860877_f3_7 | 120 | 4882 |
| 09cp61003_5860877_f2_23 | 121 | 4883 |
| 14ge10705orf5 | 122 | 4884 |
| 01cp11710orf16 | 123 | 4885 |
| 01cp11710orf5 | 124 | 4886 |
| hpy01cp11710orf_0001.aa | 125 | 4887 |
| 01cp11710orf6 | 126 | 4888 |
| 07ep11916_5913592_f3_18 | 127 | 4889 |
| 02ge10116_16803513_f2_34 | 128 | 4890 |
| 02gp20706_16803513_f1_1 | 129 | 4891 |
| 01cp11710orf18 | 130 | 4892 |
| 01cp11710orf9 | 131 | 4893 |
| 02gp20706_20365905_f2_8 | 132 | 4894 |
| hpyhp5e15276orf_0007.aa | 133 | 4895 |
| 02ge10116_23462_f2_43 | 134 | 4896 |
| 02gp20706_23632775_f3_32 | 135 | 4897 |
| 06ge10115orf4 | 136 | 4898 |
| 02ge10116_36367936_c1_19 | 137 | 4899 |
| 02ge10116_36367936_c1_92 | 138 | 4900 |
| 02ge20116orf25 | 139 | 4901 |
| 02ge10116_804550_f2_44 | 140 | 4902 |
| hpyhp5e15276orf_0005.aa | 141 | 4903 |
| 02ge41622_14875000_c2_65 | 142 | 4904 |
| hpy13ee10216orf_0031.aa | 143 | 4905 |
| 02ge11622_875260_f3_36 | 144 | 4906 |
| 13ee10216orf7 | 145 | 4907 |
| 12ge10610orf2 | 146 | 4908 |
| hpy12ge10610orf_0002.aa | 147 | 4909 |
| 05ep10815_26570332_c2_99 | 148 | 4910 |
| 06ee10207orf1 | 149 | 4911 |
| hpy06ee10207orf_0001.aa | 150 | 4912 |
| hpy36zp10248orf_0001.aa | 151 | 4913 |
| 14572133 | 152 | 4914 |
| 05ep10815_16131925_c2_97 | 153 | 4915 |
| 06ee10207orf2 | 154 | 4916 |
| 14ae21813orf2 | 155 | 4917 |
| 05ep10815_4719175_c1_83 | 156 | 4918 |
| 05ep10815_4719175_c1_115 | 157 | 4919 |
| 4960952 | 158 | 4920 |
| 04gp11701_4960952_c3_33 | 159 | 4921 |
| hp3e11168orf30 | 160 | 4922 |
| 01cp20708_214843_c2_49 | 161 | 4923 |
| hpyhp3e11168orf_0008.aa | 162 | 4924 |
| 01cp20708_4960952_c1_43 | 163 | 4925 |
| 35156938 | 164 | 4926 |
| hp5e15211_11676_f1_5 | 165 | 4927 |
| hp5e15211orf15 | 166 | 4928 |
| hp4e13462orf1 | 167 | 4929 |
| 14ep11115orf3 | 168 | 4930 |
| hpy14ep11115orf_0001.aa | 169 | 4931 |
| 11ee11408_4977193_c1_41 | 170 | 4932 |
| 05ae30220_4977193_c3_198 | 171 | 4933 |
| 917200 | 172 | 4934 |
| 05ae30220_917200_c3_172 | 173 | 4935 |
| 07ap80601orf5 | 174 | 4936 |
| 07ap80601_917200_f3_10 | 175 | 4937 |
| 05ae30220_9882767_f2_34 | 176 | 4938 |
| 05ae20220orf37 | 177 | 4939 |
| 06ae11016_4729625_c3_68 | 178 | 4940 |
| hpy07ee20513orf_0008.aa | 179 | 4941 |
| 24132293 | 180 | 4942 |
| 07ee20513orf28 | 181 | 4943 |
| 09ae10512_48768_c3_67 | 182 | 4944 |
| 06ep10306orf1 | 183 | 4945 |
| hpy06ep10306orf_0001.aa | 184 | 4946 |
| 01ae12001_24218781_f2_18 | 185 | 4947 |
| 07gp11807orf17 | 186 | 4948 |
| hpy07gp11807orf_0026.aa | 187 | 4949 |
| 11ae11922_12586675_f2_1 | 188 | 4950 |
| 14ap10221_13689381_c3_4 | 189 | 4951 |
| hp6p10903_4398263_f3_6 | 190 | 4952 |
| 05gp11901orf19 | 191 | 4953 |
| 06ap10609_12586675_f2_19 | 192 | 4954 |
| 06ap11119_24426508_f3_26 | 193 | 4955 |
| 06ap11119_24426508_f3_27 | 194 | 4956 |
| 07ae10923_24426508_f1_1 | 195 | 4957 |
| 11ge10309orf28 | 196 | 4958 |
| 4740887 | 197 | 4959 |
| hp1p11256orf7 | 198 | 4960 |
| 05ee10816_14495437_f2_13 | 199 | 4961 |
| 06ep10615_14495437_f3_47 | 200 | 4962 |
| 05ee10816_4103408_f2_11 | 201 | 4963 |
| 04ce11617orf16 | 202 | 4964 |
| hpy06ap10305orf_0002.aa | 203 | 4965 |
| hpy06ap10305orf_0001.aa | 204 | 4966 |
| 06ep10615_49068_c2_87 | 205 | 4967 |
| 23646885 | 206 | 4968 |
| 04cp11202_23646885_f2_26 | 207 | 4969 |
| 01ae12021orf8 | 208 | 4970 |
| 06gp71906_35158328_f3_85 | 209 | 4971 |
| hpy02cp11822orf_0009.aa | 210 | 4972 |
| hpy12gp11106orf_0001.aa | 211 | 4973 |
| 06gp71906_3941642_f2_70 | 212 | 4974 |
| 02ap21113orf2 | 213 | 4975 |
| 06gp71906_970325_c3_190 | 214 | 4976 |
| 14ap10815_20585777_c1_13 | 215 | 4977 |
| hp3e10349orf27 | 216 | 4978 |
| 07ap11015orf2 | 217 | 4979 |
| hp5p15580orf1 | 218 | 4980 |
| 07ep30818orf4 | 219 | 4981 |
| hpy07ep30818orf_0002.aa | 220 | 4982 |
| 07ap11015orf4 | 221 | 4983 |
| 07ap11015_23938312_f3_2 | 222 | 4984 |
| 02ep30607orf19 | 223 | 4985 |
| 07ee11402_2458267_c3_108 | 224 | 4986 |
| 14ee41924_2458267_c2_93 | 225 | 4987 |
| hp5p15641_12195281_c1_24 | 226 | 4988 |
| hp5p15612orf2 | 227 | 4989 |
| hp5p15641_21698387_c2_20 | 228 | 4990 |
| hp5p15641orf23 | 229 | 4991 |
| 02ae31010_30208317_f1_14 | 230 | 4992 |
| hp1p13947orf10 | 231 | 4993 |
| 06cp11217_4881263_f2_9 | 232 | 4994 |
| hp1p13852orf7 | 233 | 4995 |
| hpy06cp10117orf_0002.aa | 234 | 4996 |
| 06cp10117orf2 | 235 | 4997 |
| 05ce10420orf1 | 236 | 4998 |
| 09cp10224_1062966_c3_61 | 237 | 4999 |
| 09cp10224_1062966_c1_44 | 238 | 5000 |
| 09cp10224_1412715_c3_56 | 239 | 5001 |
| 01ce10516orf20 | 240 | 5002 |
| 09cp10224_1962590_f3_31 | 241 | 5003 |
| 1962590 | 242 | 5004 |
| 01ce10516orf2 | 243 | 5005 |
| hpy03ge10505orf_0004.aa | 244 | 5006 |
| 03ge10505orf2 | 245 | 5007 |
| hpy03ge10505orf_0002.aa | 246 | 5008 |
| hpyhp3e10302orf_0003.aa | 247 | 5009 |
| hp3e10302orf17 | 248 | 5010 |
| 06cp30603_679218_f2_34 | 249 | 5011 |
| hpy05gp11608orf_0002.aa | 250 | 5012 |
| hpy02ee10520orf_0001.aa | 251 | 5013 |
| hpy05gp11608orf_0001.aa | 252 | 5014 |
| 13ae10610_6522827_c3_37 | 253 | 5015 |
| 13ae10610_156411_c3_33 | 254 | 5016 |
| 04ge10816_22086531_f2_10 | 255 | 5017 |
| 04ge10816_859692_f3_12 | 256 | 5018 |
| 04ge11210orf1 | 257 | 5019 |
| 04ge11210orf2 | 258 | 5020 |
| hpy04ge11210orf_0001.aa | 259 | 5021 |
| 13ae10610_859692_c2_32 | 260 | 5022 |
| 13ap10511_12891718_c1_2 | 261 | 5023 |
| hpy13ap10511orf_0001.aa | 262 | 5024 |
| 32609403 | 263 | 5025 |
| 11ee11408_10584582_c3_51 | 264 | 5026 |
| 05gp11901orf25 | 265 | 5027 |
| 03ae10804_12609533_c1_26 | 266 | 5028 |
| 06ep10306orf10 | 267 | 5029 |
| hpyhp5e11726orf_0008.aa | 268 | 5030 |
| 06ep11509_35954752_f2_1 | 269 | 5031 |
| hp2p10625orf7 | 270 | 5032 |
| 03ae10804_14495437_c2_38 | 271 | 5033 |
| 03ee11215orf39 | 272 | 5034 |
| hp4e53394_11798952_c2_101 | 273 | 5035 |
| 06ep30223_34409437_f3_94 | 274 | 5036 |
| hp3e11024orf5 | 275 | 5037 |
| hp3e11024orf23 | 276 | 5038 |
| 06ep30223_34409437_f1_34 | 277 | 5039 |
| 06ep30223_34409437_f2_64 | 278 | 5040 |
| 29ge10111orf1 | 279 | 5041 |
| 29ge10111orf3 | 280 | 5042 |
| 06ce20610_1367157_f1_8 | 281 | 5043 |
| 14ee10308orf1 | 282 | 5044 |
| 14ee10308orf7 | 283 | 5045 |
| 02gp20814_3984818_f1_1 | 284 | 5046 |
| hpy14ee10308orf_0009.aa | 285 | 5047 |
| 11ae12004_4298568_c3_51 | 286 | 5048 |
| hpy14ee10308orf_0008.aa | 287 | 5049 |
| 11ae12004_3367666_c2_41 | 288 | 5050 |
| 06ge20501_4298568_c3_53 | 289 | 5051 |
| 10553192 | 290 | 5052 |
| 11ae80818_10553192_f2_16 | 291 | 5053 |
| 06cp11722orf16 | 292 | 5054 |
| 11ae80818_19632781_c3_57 | 293 | 5055 |
| 06cp11722orf11 | 294 | 5056 |
| 14ce61516_24609816_f2_9 | 295 | 5057 |
| 03ap21820orf8 | 296 | 5058 |
| 11ae80818_7952_c1_49 | 297 | 5059 |
| hpy07ee11519orf_0002aa | 298 | 5060 |
| hpy02ce10114orf_0004.aa | 299 | 5061 |
| 07ee11519orf1 | 300 | 5062 |
| hpy07ee11519orf_0001.aa | 301 | 5063 |
| hp7e10192_5869188_c3_17 | 302 | 5064 |
| 05ce10910_25598277_f3_3 | 303 | 5065 |
| hp7e10192_25598277_c2_15 | 304 | 5066 |
| 11ap20714_2077_c3_103 | 305 | 5067 |
| hpyhp5e15440orf_0009.aa | 306 | 5068 |
| 04gp11803orf11 | 307 | 5069 |
| 04gp11803orf13 | 308 | 5070 |
| 06ap20306_23437632_f3_9 | 309 | 5071 |
| 11ap20714_34023312_f3_46 | 310 | 5072 |
| hp3e10057orf3 | 311 | 5073 |
| 14ee11217orf4 | 312 | 5074 |
| 14ee11217orf2 | 313 | 5075 |
| 11ap20714_4960432_c3_97 | 314 | 5076 |
| hpy04gp11803orf_0013.aa | 315 | 5077 |
| 05ap21216orf4 | 316 | 5078 |
| 07ap20216_7227202_f3_10 | 317 | 5079 |
| 11ap20714_7227202_f3_40 | 318 | 5080 |
| 11ap20714_7227202_f3_43 | 319 | 5081 |
| hp1p14013_11726503_c2_20 | 320 | 5082 |
| hp1p14013orf17 | 321 | 5083 |
| 02cp10615_21908138_f1_4 | 322 | 5084 |
| hpyhp1p14013orf_0008.aa | 323 | 5085 |
| hp3p10304orf2 | 324 | 5086 |
| 02cp10615_26573462_c1_45 | 325 | 5087 |
| 31250333 | 9528 | 9640 |
| 24488537 | 9529 | 9641 |
| 16225006 | 9530 | 9642 |
| 486075 | 9531 | 9643 |
| 2458267 | 9532 | 9644 |
| 26614041 | 9533 | 9645 |
| 23567137 | 9535 | 9534 |
| 1367157 | 9535 | 9647 |
| A.2.2 Terminal phe residue and C- | | |
| terminal tyrosine cluster | | |
| 06ee10709_21675012_f1_2 | 326 | 5088 |
| 06ee10709orf2 | 327 | 5089 |
| hpy13ee10216orf_0036.aa | 328 | 5090 |
| 02ge41622_34176513_c1_50 | 329 | 5091 |
| 01cp20708_36134808_f2_11 | 330 | 5092 |
| 14ee10419orf5 | 331 | 5093 |
| hpy14ee10419orf_0001.aa | 332 | 5094 |
| 01ce10320_30273587_f3_38 | 333 | 5095 |
| 07ge11521_14160930_f3_23 | 342 | 5104 |
| hpy29ap11902orf_0001.aa | 343 | 5105 |
| 29ap11902orf1 | 344 | 5106 |
| 29ap10306orf3 | 345 | 5107 |
| hpy29ap10306orf_0003.aa | 346 | 5108 |
| 04ep41903_26757937_f1_2 | 347 | 5109 |
| 04ep41903_26757937_f3_16 | 348 | 5110 |
| 14ee21118orf2 | 349 | 5111 |
| hpy04cp11809orf_0001.aa | 350 | 5112 |
| hp3p11121_14454686_f2_1 | 351 | 5113 |
| 14ee21118orf1 | 352 | 5114 |
| hpy04cp11809orf_0002.aa | 353 | 5115 |
| 04ep41903_4101593_f2_10 | 354 | 5116 |
| 116018 | 355 | 5117 |
| 01ae12001_116018_c2_40 | 356 | 5118 |
| 06ap10609_116018_c3_50 | 357 | 5119 |
| 07gp11807orf48 | 358 | 5120 |
| 05ee10816_36126938_f3_16 | 359 | 5121 |
| 36126938 | 360 | 5122 |
| 06ep10615_36126938_f1_14 | 361 | 5123 |
| 04ce11617orf2 | 362 | 5124 |
| 01ge10801orf2 | 369 | 5131 |
| hpy01ge10801orf_0001.aa | 370 | 5132 |
| hpyhp3p11022orf_0002.aa | 371 | 5133 |
| hpyhp3p11022orf_0001.aa | 372 | 5134 |
| 02ae31010_12504512_f3_28 | 373 | 5135 |
| hpy12ap11614orf_0001.aa | 374 | 5136 |
| hpyhp5p15641orf_0002.aa | 375 | 5137 |
| hp7e10520_14728137_f1_1 | 376 | 5138 |
| 02ae31010_417818_f3_29 | 377 | 5139 |
| 01ge10801orf3 | 378 | 5140 |
| 3964593 | 379 | 5141 |
| 06cp30603_4687507_f1_7 | 380 | 5142 |
| 06cp30603_4687507_f1_9 | 381 | 5143 |
| 05cp20518orf9 | 382 | 5144 |
| hpy04ge11210orf_0003.aa | 383 | 5145 |
| 13ae10610_26855313_f3_15 | 384 | 5146 |
| 13ae10610orf2 | 385 | 5147 |
| hpy13ae10610orf_0001.aa | 386 | 5148 |
| 13ae10610orf1 | 387 | 5149 |
| 13ae10610_33726080_f1_1 | 388 | 5150 |
| 13ae10610_35912_f2_3 | 389 | 5151 |
| hp3p11086orf2 | 390 | 5152 |
| hp3p11086orf1 | 391 | 5153 |
| hp5p15575_33445317_f2_20 | 392 | 5154 |
| hpy06cp11722orf_0001.aa | 393 | 5155 |
| 06cp11722orf5 | 394 | 5156 |
| 11ae80818_7290627_c2_51 | 395 | 5157 |
| hp7e10590_26172564_c1_68 | 396 | 5158 |
| hp7e10590_7072317_c1_70 | 397 | 5159 |
| 4687507 | 9536 | 9648 |
| A.2.3 C-terminal tyrosine cluster motif | | |
| hpy02ap10310orf_0004.aa | 334 | 5096 |
| 02ap10310orf2 | 335 | 5097 |
| 02ap10310orf1 | 336 | 5098 |
| 02ap10310_30089800_f3_3 | 337 | 5099 |
| 02ap10310orf3 | 338 | 5100 |
| hpy02ap10310orf_0001.aa | 339 | 5101 |
| 02ap10310_22789651_f1_1 | 340 | 5102 |
| 04ep41903_21564052_c2_56 | 341 | 5103 |
| hpy29ep20112orf_0001.aa | 363 | 5125 |
| 29ep20112orf2 | 364 | 5126 |
| 06gp71906_20486556_f2_65 | 365 | 5127 |
| 06ep10615_961562_f2_41 | 366 | 5128 |
| 06ep10615_961562_f1_15 | 367 | 5129 |
| 06gp71906_961562_f3_117 | 368 | 5130 |
| 06gp10108orf2 | 398 | 5160 |
| hpy06gp10108orf_0003.aa | 399 | 5161 |
| 09cp61003_492187_c2_80 | 400 | 5162 |
| 01ce61016_492187_c3_120 | 401 | 5163 |
| 01ce10516orf21 | 402 | 5164 |
| 09cp10224_429510_c2_46 | 403 | 5165 |
| 07cp21714orf2 | 404 | 5166 |
| 07cp21714orf1 | 405 | 5167 |
| 07cp21714orf3 | 406 | 5168 |
| 07ce11019_22051291_f1_1 | 407 | 5169 |
| 14gp11423_26803801_f1_1 | 408 | 5170 |
| 06cp11217_19720300_f3_11 | 409 | 5171 |
| hp4e53394_22864682_c2_86 | 410 | 5172 |
| hp4e53394_19720300_c3_98 | 411 | 5173 |
| hp1p13852orf6 | 412 | 5174 |
| hp4e53394_26209843_c3_98 | 413 | 5175 |
| hp4e53394_26756900_c3_103 | 414 | 5176 |
| hp6p12244_5273452_c2_82 | 415 | 5177 |
| 01cp11414orf2 | 416 | 5178 |
| 07gp11909_26460892_f2_6 | 424 | 5186 |
| 02ap11117_26460892_f1_15 | 425 | 5187 |
| A.2.4 Via homology | | |
| hp6e12267_4721061_c1_41 | 417 | 5179 |
| 09cp61003_4721061_f1_16 | 418 | 5180 |
| 4721061 | 419 | 5181 |
| 12ge20305orf2 | 420 | 5182 |
| 09cp11003_5945252_f2_4 | 421 | 5183 |
| 09cp61003_5945252_f1_5 | 422 | 5184 |
| 14ge10705orf3 | 423 | 5185 |
| 01ae11010_40688_c2_38 | 426 | 5188 |
| hp4p33322_40688_c1_38 | 427 | 5189 |
| hp3e11075orf3 | 428 | 5190 |
| 14ce31519orf1 | 429 | 5191 |
| 14ce11519orf4 | 430 | 5192 |
| 04ep41903_4538588_f3_21 | 431 | 5193 |
| 04ep41903_4538588_f2_20 | 432 | 5194 |
| 05ae11108orf1 | 433 | 5195 |
| hpy05ae11108orf_0001.aa | 434 | 5196 |
| 03ce10801orf1 | 435 | 5197 |
| hpy03ce10801orf_0001.aa | 436 | 5198 |
| 03ce10801orf4 | 437 | 5199 |
| 05ae30220_4487562_c1_101 | 438 | 5200 |
| 05ae30220_26834500_c2_164 | 439 | 5201 |
| 5083193 | 440 | 5202 |
| 05ae30220_5083193_c3_165 | 441 | 5203 |
| 07ap80601orf8 | 442 | 5204 |
| 07ap80601_5083193_f3_8 | 443 | 5205 |
| hp5p15212_13729635_c3_35 | 444 | 5206 |
| 06gp10409_3398427_f2_12 | 445 | 5207 |
| 13ae10511orf3 | 446 | 5208 |
| hpy13ae10511orf_0001.aa | 447 | 5209 |
| 06gp10409_3398427_f2_12 | 448 | 5210 |
| 06gp71906_2003143_c3_217 | 449 | 5211 |
| hpy16cp30109orf_0001.aa | 450 | 5212 |
| 4490717 | 451 | 5213 |
| hpy16cp30109orf_0002.aa | 452 | 5214 |
| 16cp30109orf6 | 453 | 5215 |
| 04cp11202_4490717_f2_29 | 454 | 5216 |
| hpy06ce31218orf_0001.aa | 455 | 5217 |
| 14ee41924_1046877_c3_104 | 456 | 5218 |
| 07ee11402_1046877_c3_100 | 457 | 5219 |
| hpy02ep30607orf_0014.aa | 458 | 5220 |
| hpy02ep30607orf_0012.aa | 459 | 5221 |
| 02ep30607orf27 | 460 | 5222 |
| 14ee41924_23527267_c3_107 | 461 | 5223 |
| 07ee11402_10759567_c2_86 | 462 | 5224 |
| hpy02ep30607orf_0015.aa | 463 | 5225 |
| 14ee41924_33203917_c2_85 | 464 | 5226 |
| 3906963 | 465 | 5227 |
| 04ee11108_3906963_f1_7 | 466 | 5228 |
| 27ze10351orf5 | 467 | 5229 |
| 06cp30603_23476568_c1_44 | 468 | 5230 |
| 978477 | 469 | 5231 |
| 05cp20518orf39 | 470 | 5232 |
| 06cp30603_978517_c3_137 | 471 | 5233 |
| hpy04gp11213orf_0009.aa | 472 | 5234 |
| hpy02ce12007orf_0003.aa | 473 | 5235 |
| hpy02ce12007orf_0002.aa | 474 | 5236 |
| hp1p13939orf13 | 475 | 5237 |
| 05ce10208_4766691_c2_18 | 476 | 5238 |
| 4766691 | 477 | 5239 |
| hpyhp1p13939orf_0001.aa | 478 | 5240 |
| hp1p13939_21641016_f1_1 | 479 | 5241 |
| hp4p62853_4766691_f3_23 | 480 | 5242 |
| 03xe11215orf7 | 481 | 5243 |
| 03xe11215orf8 | 482 | 5244 |
| 02ae11612_22477267_f2_27 | 483 | 5245 |
| 02gp20814_24415958_f3_9 | 484 | 5246 |
| 14ee10308orf4 | 485 | 5247 |
| 09cp21607_31262_c2_11 | 486 | 5248 |
| 31262 | 487 | 5249 |
| 07gp31516orf4 | 488 | 5250 |
| 35887 | 489 | 5251 |
| 11ap20714_4797137_f3_45 | 490 | 5252 |
| hp3e10057orf2 | 491 | 5253 |
| A.2.5 Other outer membrane proteins | | |
| 04ep41903_23867687_c2_32 | 492 | 5254 |
| 23867687 | 493 | 5255 |
| 09ae11601orf11 | 494 | 5256 |
| 06ep10615_9842_f3_46 | 495 | 5257 |
| 06ep10615_189160_f3_47 | 496 | 5258 |
| hpy05ep11717orf_0008.aa | 497 | 5259 |
| 05ep11717orf2 | 498 | 5260 |
| 06ep10615_9842_f1_5 | 499 | 5261 |
| hpy02ae20216orf_0001.aa | 500 | 5262 |
| hpy02ce30710orf_0006.aa | 501 | 5263 |
| 02ae20216orf2 | 502 | 5264 |
| hpyhp1e10506orf_0008.aa | 503 | 5265 |
| 05cp21223_29308518_c2_24 | 504 | 5266 |
| 06ep30223_104052_c2_138 | 505 | 5267 |
| 05cp21223_23831400_c1_22 | 506 | 5268 |
| hp1e10506orf22 | 507 | 5269 |
| 14cp10923_4414000_f3_11 | 508 | 5270 |
| 4414000 | 509 | 5271 |
| 14cp10923orf8 | 510 | 5272 |
| A.3 Inner membrane | | |
| A.3.1 Proteins involved in transport | | |
| hp6e12267_1069213_f3_31 | 511 | 5273 |
| 12ge20305orf35 | 512 | 5274 |
| 11132778 | 513 | 5275 |
| hp6e12267_11132778_c2_48 | 514 | 5276 |
| 12ge10305orf16 | 515 | 5277 |
| 20032561 | 516 | 5278 |
| 09ap20802orf27 | 517 | 5279 |
| hp2p10272_4100312_c2_35 | 518 | 5280 |
| 02ge10116_24238762_c1_22 | 519 | 5281 |
| 24238762 | 520 | 5282 |
| 02ge20116orf28 | 521 | 5283 |
| hpyhp3e11168orf_0011.aa | 522 | 5284 |
| 23492181 | 523 | 5285 |
| 23853165 | 524 | 5286 |
| hp3e11168orf29 | 525 | 5287 |
| 04gp11701_23442187_c3_32 | 526 | 5288 |
| 14ce31519_15635927_f3_15 | 527 | 5289 |
| 02ce10809orf6 | 528 | 5290 |
| 02ce10809orf16 | 529 | 5291 |
| 14ce31519_24650009_c1_17 | 530 | 5292 |
| 04ep41903_16667055_c1_37 | 531 | 5293 |
| 04ep41903_19689182_c1_43 | 532 | 5294 |
| 2461062 | 533 | 5295 |
| 1179838 | 534 | 5296 |
| 05ae30220_4954526_f3_53 | 535 | 5297 |
| 05ae20220orf32 | 536 | 5298 |
| 05ae20220orf54 | 537 | 5299 |
| 05ae30220_1179838_f2_53 | 538 | 5300 |
| 5878208 | 539 | 5301 |
| 27ze10351orf29 | 540 | 5302 |
| 23728388 | 541 | 5303 |
| 27ze10351orf24 | 542 | 5304 |
| 04ee11108_23728388_c2_22 | 543 | 5305 |
| 35345228 | 544 | 5306 |
| 04ee11108_3914683_c3_27 | 545 | 5307 |
| 27ze10351orf18 | 546 | 5308 |
| hpy27ze10351orf_0001.aa | 547 | 5309 |
| 05ae30220_3914683_c1_106 | 548 | 5310 |
| 11924177 | 549 | 5311 |
| 04ee11108_56313_f1_9 | 550 | 5312 |
| 27ze10351orf7 | 551 | 5313 |
| 01ae12001_14714687_f2_16 | 552 | 5314 |
| 14714687 | 553 | 5315 |
| 07gp11807orf9 | 554 | 5316 |
| 07gp11807orf29 | 555 | 5317 |
| 19531291 | 556 | 5318 |
| 42683 | 557 | 5319 |
| 07gp11807orf49 | 558 | 5320 |
| 01ae12001_19536375_c2_41 | 559 | 5321 |
| 01ae12001_3319687_f1_10 | 560 | 5322 |
| 3319687 | 561 | 5323 |
| 07gp11807orf25 | 562 | 5324 |
| 5875152 | 563 | 5325 |
| 01ae12001_5875152_f2_15 | 564 | 5326 |
| 07gp11807orf8 | 565 | 5327 |
| 06ap11119_9860256_f1_4 | 566 | 5328 |
| hp6p10723orf11 | 567 | 5329 |
| 01ce11513orf29 | 568 | 5330 |
| 01ce11513orf33 | 569 | 5331 |
| 1464715 | 570 | 5332 |
| 06cp11118_1464715_c2_19 | 571 | 5333 |
| 06cp11118_26823300_c1_15 | 572 | 5334 |
| 06cp11118_213561_c1_15 | 573 | 5335 |
| 01ce11513orf22 | 574 | 5336 |
| 12ap11619orf1 | 575 | 5337 |
| hpy12ap11619orf_0001.aa | 576 | 5338 |
| 06cp11118_391525_c2_18 | 577 | 5339 |
| 06ep10615_15659509_c1_51 | 578 | 5340 |
| 06ep10615_5203401_c3_87 | 579 | 5341 |
| 11cp10113orf4 | 580 | 5342 |
| hpy11cp10113orf_0001.aa | 581 | 5343 |
| 29zp10241orf4 | 582 | 5344 |
| 14cp20718_20210817_f1_1 | 583 | 5345 |
| 07ap61111_33330468_f1_6 | 584 | 5346 |
| 12ae11420orf1 | 585 | 5347 |
| hpy12ae11420orf_0001.aa | 586 | 5348 |
| 14ee41924_24001568_f2_40 | 587 | 5349 |
| 07ee11402_24001568_f1_15 | 588 | 5350 |
| 07ae11611orf1 | 589 | 5351 |
| hpy07ae11611orf_0001.aa | 590 | 5352 |
| 07ee11402_32431453_f1_1 | 591 | 5353 |
| 24609593 | 592 | 5354 |
| 14ce11113orf1 | 593 | 5355 |
| hpy14ce11113orf_0001.aa | 594 | 5356 |
| 07ee11402_5127343_f3_2 | 595 | 5357 |
| hpy14ae20424orf_0002.aa | 596 | 5358 |
| 04ae61517_1048812_f1_1 | 597 | 5359 |
| 04ae21517orf1 | 598 | 5360 |
| 04ae61517_24802342_f2_3 | 599 | 5361 |
| 07ee50709_411457_f2_68 | 600 | 5362 |
| 10723412 | 601 | 5363 |
| 02ae31010_4455467_c2_84 | 602 | 5364 |
| 02ce11022orf8 | 603 | 5365 |
| 07ae32002orf3 | 604 | 5366 |
| hpy07ae32002orf_0002.aa | 605 | 5367 |
| hp4p13446orf10 | 606 | 5368 |
| hp6p10904_22069680_f3_12 | 607 | 5369 |
| hpy06gp11202orf_0001.aa | 608 | 5370 |
| hpy06gp111202orf_0002.aa | 609 | 5371 |
| 33399142 | 610 | 5372 |
| 06gp11202orf7 | 611 | 5373 |
| hpy06gp11202orf_0003.aa | 612 | 5374 |
| 09ce10413_24428452_f1_5 | 613 | 5375 |
| hp6p10904_6726062_f3_13 | 614 | 5376 |
| 09ce10413_26734687_f3_23 | 615 | 5377 |
| hpyhp3e10302orf_0001.aa | 616 | 5378 |
| 29479681 | 617 | 5379 |
| 05cp20518orf33 | 618 | 5380 |
| 06cp30603_22666625_f3_58 | 619 | 5381 |
| 06cp30603_664083_c1_94 | 620 | 5382 |
| 06cp30603orf10 | 621 | 5383 |
| 06cp30603orf9 | 622 | 5384 |
| 06cp30603_36359687_c3_78 | 623 | 5385 |
| 06cp30603_36359687_c3_161 | 624 | 5386 |
| 09cp10713_36359687_c1_119 | 625 | 5387 |
| 16406265 | 626 | 5388 |
| 12ge10321_4691563_f2_6 | 627 | 5389 |
| hp4p11352orf4 | 628 | 5390 |
| 12ae10622_24406450_f1_9 | 629 | 5391 |
| 179677 | 630 | 5392 |
| 03ae10804_22459680_f1_4 | 631 | 5393 |
| hp5e11726orf7 | 632 | 5394 |
| hp1e80523_3906693_f1_2 | 633 | 5395 |
| hp3e10929orf1 | 634 | 5396 |
| hp3e11188_26359712_f2_3 | 635 | 5397 |
| 06ep11108orf13 | 636 | 5398 |
| hpy06ep11108orf_0009.aa | 637 | 5399 |
| 01ep10216orf9 | 638 | 5400 |
| hp4e13394_19656577_f3_57 | 639 | 5401 |
| hp5e13045orf1 | 640 | 5402 |
| 02ae11611orf11 | 641 | 5403 |
| hp$$0$$ae11611orf_0005.aa | 642 | 5404 |
| hp4e53394_24744002_f1_1 | 643 | 5405 |
| hp5e13045_24744002_f1_1 | 644 | 5406 |
| hp1p13939_817683_c3_36 | 645 | 5407 |
| hp4p62853_15818754_c2_42 | 646 | 5408 |
| 783432 | 647 | 5409 |
| 05cp11911orf27 | 648 | 5410 |
| 02ae11612_14641535_c1_57 | 649 | 5411 |
| 02ae11612_4338438_c3_91 | 650 | 5412 |
| 4338438 | 651 | 5413 |
| 05cp11911orf41 | 652 | 5414 |
| hp5p15575_292564_c2_38 | 653 | 5415 |
| hp5e15084orf6 | 654 | 5416 |
| hp6e10491_31757638_f1_1 | 655 | 5417 |
| 24416083 | 656 | 5418 |
| 13ae10712orf4 | 657 | 5419 |
| 13ae10712_24416083_f2_10 | 658 | 5420 |
| 05gp20111orf1 | 659 | 5421 |
| 06ce20610_4086003_c1_28 | 660 | 5422 |
| 13ae10712orf9 | 661 | 5423 |
| 22379952 | 662 | 5424 |
| 13ae10712_4725342_f1_4 | 663 | 5425 |
| 03ap21820orf5 | 664 | 5426 |
| 03ap21820orf17 | 665 | 5427 |
| 11ae80818_36131282_c3_64 | 666 | 5428 |
| 10353192 | 667 | 5429 |
| 12ap10324_16422591_f3_4 | 668 | 5430 |
| 12ap10324orf2 | 669 | 5431 |
| 01ce11513orf21 | 670 | 5432 |
| 02ep20506_4882763_c2_19 | 671 | 5433 |
| hp7p10287_4882763_f2_9 | 672 | 5434 |
| 33203192 | 673 | 5435 |
| 06ap11418_33203192_f1_1 | 674 | 5436 |
| hp5e15440orf16 | 675 | 5437 |
| 03ae11503_4532892_c2_19 | 676 | 5438 |
| 03ae11503orf10 | 677 | 5439 |
| 4826401 | 678 | 5440 |
| 01ae22001orf5 | 679 | 5441 |
| 01ae22001orf2 | 680 | 5442 |
| 01ae22001orf1 | 681 | 5443 |
| 02cp10615_33877086_c3_61 | 682 | 5444 |
| 22453166 | 9537 | 9649 |
| 875042 | 9538 | 9650 |
| 19536375 | 9539 | 9651 |
| 10675632 | 9540 | 9652 |
| 24218968 | 9541 | 9653 |
| 13726562 | 9542 | 9654 |
| 36131282 | 9543 | 9655 |
| 16422591 | 9544 | 9656 |
| 4882763 | 9545 | 9657 |
| 36573502 | 9546 | 9658 |
| A.3.1.1 Proteins involved in amino acid | | |
| metabolism & transport | | |
| 2042312 | 683 | 5445 |
| 02gp20706_2042312_c1_44 | 684 | 5446 |
| 01cp11710orf34 | 685 | 5447 |
| 04ap20119orf5 | 686 | 5448 |
| 01cp10707orf2 | 687 | 5449 |
| 02ge11810_32538290_f1_4 | 688 | 5450 |
| 02ge11810_867187_f3_10 | 689 | 5451 |
| 02ge11810_867187_f3_9 | 690 | 5452 |
| 02ce10809orf2 | 691 | 5453 |
| 02ce10809orf4 | 692 | 5454 |
| 14ce31519_16042_f2_11 | 693 | 5455 |
| 02ce10809orf5 | 694 | 5456 |
| 14ce31519_9960937_f3_14 | 695 | 5457 |
| 24215 | 696 | 5458 |
| 06gp10409_80130_c2_24 | 697 | 5459 |
| 06gp10409_80130_c1_24 | 698 | 5460 |
| 04ep10811orf4 | 699 | 5461 |
| 02ae31522orf4 | 700 | 5462 |
| 02ae31522orf3 | 701 | 5463 |
| 14gp11720_3906693_c3_4 | 702 | 5464 |
| 02ae41522_3906693_c2_3 | 703 | 5465 |
| 35269000 | 704 | 5466 |
| 02ae11611orf1 | 705 | 5467 |
| hpy02ae11611orf_0001.aa | 706 | 5468 |
| hp4e53394_30602256_c3_124 | 707 | 5469 |
| hp5e13045_4454655_c3_14 | 708 | 5470 |
| hp4e53394_4460905_c3_121 | 709 | 5471 |
| hpy14ce10720orf_0001.aa | 710 | 5472 |
| 01ee11622orf2 | 711 | 5473 |
| hpy01ee11622orf_0001.aa | 712 | 5474 |
| hp5p15575_1955465_c3_41 | 713 | 5475 |
| 02cp11813orf1 | 714 | 5476 |
| hp6e50324_24507825_c3_12 | 715 | 5477 |
| 03ae10804orf2 | 716 | 5478 |
| hp5e11726orf2 | 717 | 5479 |
| hp5e11726orf4 | 718 | 5480 |
| 36203402 | 719 | 5481 |
| 03ae10804_192208_f2_9 | 720 | 5482 |
| 5083577 | 9547 | 9659 |
| 289711 | 9548 | 9660 |
| “A.3.1.2 Involved in nucleotide, lipid, or | | |
| cofactor metabolism & transport” | | |
| 02gp20706_24415918_f3_20 | 721 | 5483 |
| 01cp11710orf2 | 722 | 5484 |
| 12ae11404orf16 | 723 | 5485 |
| 12ae11404orf17 | 724 | 5486 |
| 12ae11404_19963577_c3_15 | 725 | 5487 |
| 12ae11404orf14 | 726 | 5488 |
| 12ae11404orf15 | 727 | 5489 |
| 12ae11404orf11 | 728 | 5490 |
| hpy12ae11404orf_0011.aa | 729 | 5491 |
| 05ee11015_26375799_c1_10 | 730 | 5492 |
| 12ae11404_992256_c3_14 | 731 | 5493 |
| 09ae10512orf3 | 732 | 5494 |
| 09ae10512orf1 | 733 | 5495 |
| hpy09ae10512orf_0001.aa | 734 | 5496 |
| 07ee20513orf11 | 735 | 5497 |
| 09ae10512_23942135_f1_1 | 736 | 5498 |
| 06ae11016_14063177_f3_23 | 737 | 5499 |
| 06gp71906_25478192_c1_131 | 738 | 5500 |
| hp3e11122orf3 | 739 | 5501 |
| 29gp20307orf1 | 740 | 5502 |
| hpy11ee21702orf_0001.aa | 741 | 5503 |
| 11ee21702orf1 | 742 | 5504 |
| hp5p15641_4335890_c2_27 | 743 | 5505 |
| 02ae31010_5085162_c1_47 | 744 | 5506 |
| 07ce10203orf11 | 745 | 5507 |
| hpy05cp20518orf_0001.aa | 746 | 5508 |
| 24824087 | 747 | 5509 |
| hpy05cp20518orf_0002.aa | 748 | 5510 |
| 06cp30603orf11 | 749 | 5511 |
| 06cp30603_14663917_c1_51 | 750 | 5512 |
| 06cp30603_24256503_c1_110 | 751 | 5513 |
| 01ge10214orf1 | 752 | 5514 |
| hpy01ge10214orf_0001.aa | 753 | 5515 |
| hp3e11188_24417800_f2_4 | 754 | 5516 |
| 03ee11215_22367062_c2_29 | 755 | 5517 |
| 03ee11215orf38 | 756 | 5518 |
| 01ge10203orf1 | 757 | 5519 |
| 01ge10203orf3 | 758 | 5520 |
| 01ge10203_4875375_f2_3 | 759 | 5521 |
| hp3e11024orf8 | 760 | 5522 |
| hp3e11024orf18 | 761 | 5523 |
| 06ep30223_204582_f1_39 | 762 | 5524 |
| 02cp11721orf20 | 763 | 5525 |
| hp7e10100_5350012_c2_18 | 764 | 5526 |
| 24806290 | 9549 | 9661 |
| A.3.1.3 Proteins involved in inorganic | | |
| ion transport | | |
| 01cp11710orf28 | 765 | 5527 |
| 01cp11710orf37 | 766 | 5528 |
| 02gp20706_24645837_c3_64 | 767 | 5529 |
| 02gp20706_4879625_f2_9 | 768 | 5530 |
| 02ge10116_4879625_f3_58 | 769 | 5531 |
| 01cp11710orf10 | 770 | 5532 |
| 06gp10409orf9 | 771 | 5533 |
| 04ep10811orf2 | 772 | 5534 |
| 06gp10409orf8 | 773 | 5535 |
| 06gp10409orf7 | 774 | 5536 |
| hpy06gp10409orf_0004.aa | 775 | 5537 |
| 06gp10409_21751016_c3_26 | 776 | 5538 |
| 06gp10409_4881318_c1_21 | 777 | 5539 |
| 06gp10409_4881318_c3_30 | 778 | 5540 |
| 14ce20219orf2 | 779 | 5541 |
| 14ce20219orf1 | 780 | 5542 |
| 14ce20219orf4 | 781 | 5543 |
| 06gp71906_667968_f3_66 | 782 | 5544 |
| 09cp10502orf18 | 783 | 5545 |
| 09cp10502orf11 | 784 | 5546 |
| 07ae11008_250652_c3_59 | 785 | 5547 |
| 07ae11008_15806538_c1_38 | 786 | 5548 |
| 16406581 | 787 | 5549 |
| 07ce11019_3931943_c1_11 | 788 | 5550 |
| 07cp21714orf13 | 789 | 5551 |
| 114505 | 790 | 5552 |
| 11ap11902orf3 | 791 | 5553 |
| hpy11ap11902orf_0001.aa | 792 | 5554 |
| 11ae80818_11188791_c3_60 | 793 | 5555 |
| 06cp11722orf12 | 794 | 5556 |
| 11ae80818_25593768_c1_44 | 795 | 5557 |
| 14cp11908_25593768_c3_97 | 796 | 5558 |
| 11ae80818_6828218_f2_19 | 797 | 5559 |
| 6828218 | 798 | 5560 |
| 06cp11722orf21 | 799 | 5561 |
| 24645837 | 9550 | 9662 |
| 3203142 | 9551 | 9663 |
| 34666680 | 9552 | 9664 |
| 22441050 | 9553 | 9665 |
| 26258562 | 9554 | 9666 |
| A.3.1.4 Proteins involved in | | |
| carbohydrate metabolism & transport | | |
| 21742157 | 800 | 5562 |
| 07ce11409orf4 | 801 | 5563 |
| 14ee41924_5865675_f1_18 | 802 | 5564 |
| A.3.2 Proteins involved in outer | | |
| membrane and cell wall formation | | |
| hp6p80503_235782_f3_18 | 803 | 5565 |
| 01ce11016orf19 | 804 | 5566 |
| 01ce11016orf22 | 805 | 5567 |
| hp6p80503_26593953_f1_4 | 806 | 5568 |
| 01ce61016_26593953_c1_113 | 807 | 5569 |
| 14ce11503orf3 | 808 | 5570 |
| 09cp11003_24646926_c2_13 | 809 | 5571 |
| 14ge10705orf11 | 810 | 5572 |
| hpy14ge10705orf_0005.aa | 811 | 5573 |
| 09cp61003_29322967_c1_75 | 812 | 5574 |
| 04ap20119orf2 | 813 | 5575 |
| 02ge11810_35282625_f3_9 | 814 | 5576 |
| 06cp20302orf1 | 815 | 5577 |
| hpy06cp20302orf_0001.aa | 816 | 5578 |
| 06ep11202_24219093_c3_30 | 817 | 5579 |
| hpy06ap10209orf_0005.aa | 818 | 5580 |
| 06ap10209orf3 | 819 | 5581 |
| 25992137 | 820 | 5582 |
| 04ge11713orf37 | 821 | 5583 |
| hpy04ge11713orf_0020.aa | 822 | 5584 |
| 04cp11202_3178500_c1_76 | 823 | 5585 |
| 04cp11202_3178500_c3_110 | 824 | 5586 |
| 11ep12011orf5 | 825 | 5587 |
| 11ep12011orf9 | 826 | 5588 |
| 06gp71906_3945965_c2_153 | 827 | 5589 |
| 13ap11517orf25 | 828 | 5590 |
| 13ap11517orf20 | 829 | 5591 |
| 06cp10411orf6 | 830 | 5592 |
| 12gp31106_5267037_c1_43 | 831 | 5593 |
| 06gp71906_5267037_f3_106 | 832 | 5594 |
| 07ae11008_24409577_c3_56 | 833 | 5595 |
| 07ae11008_24409577_c1_37 | 834 | 5596 |
| 25605166 | 835 | 5597 |
| 04ee11108_954683_c1_15 | 836 | 5598 |
| 27ze10351orf17 | 837 | 5599 |
| 14gp12015orf16 | 838 | 5600 |
| hp2e10229orf2 | 839 | 5601 |
| 14gp12015orf13 | 840 | 5602 |
| 06ep30223_4698838_f2_55 | 841 | 5603 |
| 03ap21820orf9 | 842 | 5604 |
| 03ap21820orf3 | 843 | 5605 |
| 03ap21820orf11 | 844 | 5606 |
| 11ae80818_24415917_c1_45 | 845 | 5607 |
| hpy02cp11721orf_0004.aa | 846 | 5608 |
| 02cp11721orf14 | 847 | 5609 |
| hp7e10100_1359450_c1_14 | 848 | 5610 |
| 22460468 | 9555 | 9667 |
| 23468781 | 9556 | 9668 |
| 495312 | 9557 | 9669 |
| 5267037 | 9558 | 9670 |
| 4698838 | 9559 | 9671 |
| 24415917 | 9560 | 9672 |
| A.3.3 Proteins involved in energy | | |
| conversion | | |
| hpy07ge20415orf_0003.aa | 849 | 5611 |
| hp7e10429_7132780_c2_38 | 850 | 5612 |
| 06ge10217_32615840_f3_1 | 851 | 5613 |
| 06ge10217orf1 | 852 | 5614 |
| 01ce11016orf8 | 853 | 5615 |
| hp6p80503_6845042_c2_25 | 854 | 5616 |
| 01ce61016_24492212_f1_1 | 855 | 5617 |
| 01ce11016orf1 | 856 | 5618 |
| hp6p80503_992202_c1_21 | 857 | 5619 |
| hpy04ce11408orf_0002.aa | 858 | 5620 |
| hp2p10272_5211682_c3_44 | 859 | 5621 |
| 04ce11408orf2 | 860 | 5622 |
| 02ap11117orf1 | 861 | 5623 |
| hp6p21623_6906328_c2_51 | 862 | 5624 |
| 09ge11703orf1 | 863 | 5625 |
| 14ce31519_29339030_c2_27 | 864 | 5626 |
| 09ge11703orf2 | 865 | 5627 |
| hpy09ge11703orf_0001.aa | 866 | 5628 |
| hpyhp3p10366orf_0001.aa | 867 | 5629 |
| hp3p10366orf2 | 868 | 5630 |
| 14ce31519_3364062_c1_21 | 869 | 5631 |
| 06cp11118orf5 | 870 | 5632 |
| 06cp11118_24620968_c3_24 | 871 | 5633 |
| hpy06cp11118orf_0002.aa | 872 | 5634 |
| 06cp11118orf8 | 873 | 5635 |
| 06cp11118_25445342_c2_20 | 874 | 5636 |
| hpy06cp11118orf_0006.aa | 875 | 5637 |
| 16412593 | 876 | 5638 |
| 06cp11118orf6 | 877 | 5639 |
| 06cp11118_4532588_c3_23 | 878 | 5640 |
| 04ge11613orf5 | 879 | 5641 |
| 04ge11613orf4 | 880 | 5642 |
| hp5e15419orf2 | 881 | 5643 |
| 06gp10409_24406465_c3_27 | 882 | 5644 |
| 3953143 | 883 | 5645 |
| 02ae31010_29308453_f2_25 | 884 | 5646 |
| hp1p13947orf2 | 885 | 5647 |
| 01ce10516orf18 | 886 | 5648 |
| hpy01ce10516orf_0011.aa | 887 | 5649 |
| 01ce10516orf17 | 888 | 5650 |
| 09cp10224_23570302_c1_37 | 889 | 5651 |
| 09cp10224_32042937_c1_39 | 890 | 5652 |
| 01ce10516orf25 | 891 | 5653 |
| hp1p11244orf5 | 892 | 5654 |
| 09cp10224_35585952_c1_41 | 893 | 5655 |
| 01ce10516orf24 | 894 | 5656 |
| 01ce10516orf19 | 895 | 5657 |
| 01ce10516orf15 | 896 | 5658 |
| 09cp10224_4484718_c1_38 | 897 | 5659 |
| 11cp71403orf5 | 898 | 5660 |
| 09ze10333_4962817_f3_12 | 899 | 5661 |
| 16ae10113orf1 | 900 | 5662 |
| 16ae10113orf3 | 901 | 5663 |
| hpy07ee11620orf_0001.aa | 902 | 5664 |
| 16ae10113orf2 | 903 | 5665 |
| hpy16ae10113orf_0001.aa | 904 | 5666 |
| 09ze10333_4962817_f3_11 | 905 | 5667 |
| 05ae20220orf93 | 906 | 5668 |
| hp2e51852_23711552_c3_4 | 907 | 5669 |
| 16131887 | 908 | 5670 |
| 2082012 | 909 | 5671 |
| 09ap11406orf14 | 910 | 5672 |
| 09ap11406orf5 | 911 | 5673 |
| hp4e13394_1353438_c2_103 | 912 | 5674 |
| 02ap71220orf3 | 913 | 5675 |
| 02ap71220orf2 | 914 | 5676 |
| hp4e13394_5964452_c2_97 | 915 | 5677 |
| hp4e13394_15828963_c2_90 | 916 | 5678 |
| 6093906 | 917 | 5679 |
| hp4e13394_2042837_c3_122 | 918 | 5680 |
| 09ap11406orf15 | 919 | 5681 |
| 11ce11603orf14 | 920 | 5682 |
| 11ce11603orf23 | 921 | 5683 |
| hp4e13394_23446016_c3_119 | 922 | 5684 |
| hp4e13394_23915877_c1_75 | 923 | 5685 |
| 23915877 | 924 | 5686 |
| 11ce11603orf6 | 925 | 5687 |
| hpy11ce11603orf_0004.aa | 926 | 5688 |
| 11ce11603orf2 | 927 | 5689 |
| 11ce11603orf19 | 928 | 5690 |
| hp4e13394_29977187_c1_72 | 929 | 5691 |
| hpy09ap11406orf_0001.aa | 930 | 5692 |
| 1204418 | 931 | 5693 |
| 11ce11603orf16 | 932 | 5694 |
| hp4e13394_32218775_c2_102 | 933 | 5695 |
| 4035783 | 934 | 5696 |
| hp4e13394_3908568_c3_120 | 935 | 5697 |
| 11ce11603orf25 | 936 | 5698 |
| 11ce11603orf13 | 937 | 5699 |
| 11ce11603orf12 | 938 | 5700 |
| hpy11ce11603orf_0008.aa | 939 | 5701 |
| 11ce11603orf3 | 940 | 5702 |
| 11ce11603orf22 | 941 | 5703 |
| hp4e13394_490786_c1_73 | 942 | 5704 |
| hp4e13394_677292_c3_121 | 943 | 5705 |
| 11ce11603orf26 | 944 | 5706 |
| hp4e53394_29422028_c1_84 | 945 | 5707 |
| hp1e10523orf1 | 946 | 5708 |
| hp1e10523orf2 | 947 | 5709 |
| hpyhp1e10523orf_0001.aa | 948 | 5710 |
| 01ce11104_4589005_c3_14 | 949 | 5711 |
| hp5p15575orf14 | 950 | 5712 |
| 05cp10201orf1 | 951 | 5713 |
| hpy05cp10201orf_0001.aa | 952 | 5714 |
| hp5p15575orf16 | 953 | 5715 |
| hpyhp5p15575orf_0007.aa | 954 | 5716 |
| 13ep10801orf2 | 955 | 5717 |
| hp5p15575_24246012_c2_39 | 956 | 5718 |
| hp6p10590_30521093_f2_14 | 957 | 5719 |
| 11ge11422orf3 | 958 | 5720 |
| 07ce11206orf1 | 959 | 5721 |
| hp6p10590_4817177_f3_22 | 960 | 5722 |
| hpy05gp20111orf_0005.aa | 961 | 5723 |
| 05gp20111orf12 | 962 | 5724 |
| 06ce20610_4331338_f3_18 | 963 | 5725 |
| 01gp11016_17070306_c2_16 | 964 | 5726 |
| hpy01gp11016orf_0003.aa | 965 | 5727 |
| 01gp11016orf20 | 966 | 5728 |
| 01gp11016orf16 | 967 | 5729 |
| 01gp11016_12583568_c1_10 | 968 | 5730 |
| 01gp11016_1442187_c3_18 | 969 | 5731 |
| 01gp11016orf19 | 970 | 5732 |
| hp7e10434_1442187_f3_19 | 971 | 5733 |
| hp7e10434_210827_f2_11 | 972 | 5734 |
| hpy01gp11016orf_0006.aa | 973 | 5735 |
| 12ap10324orf7 | 974 | 5736 |
| 12ap10324_23531562_f2_2 | 975 | 5737 |
| 12ap10324_16052038_f1_1 | 976 | 5738 |
| 12ap10324orf1 | 977 | 5739 |
| hpy12ap10324orf_0001.aa | 978 | 5740 |
| hp7e30434_24651068_f3_32 | 979 | 5741 |
| 01gp11016orf11 | 980 | 5742 |
| 01gp11016orf12 | 981 | 5743 |
| hpy01gp11016orf_0001.aa | 982 | 5744 |
| hp7e10434_24744012_f2_14 | 983 | 5745 |
| hp7e30434_24744012_f3_31 | 984 | 5746 |
| 01gp11016orf13 | 985 | 5747 |
| 01gp11016_4103403_c2_13 | 986 | 5748 |
| 5869090 | 987 | 5749 |
| 01gp11016_5869090_c2_14 | 988 | 5750 |
| 01gp11016orf14 | 989 | 5751 |
| 06ap11418_31813750_c2_24 | 990 | 5752 |
| hp5e15440orf8 | 991 | 5753 |
| 11ap20714_4692_f3_30 | 992 | 5754 |
| hp6p12158_3917638_c1_2 | 993 | 5755 |
| 09ap10614orf1 | 994 | 5756 |
| hp5e25328_3917638_c3_138 | 995 | 5757 |
| 32236462 | 9561 | 9673 |
| 423131 | 9562 | 9674 |
| 14455461 | 9563 | 9675 |
| 26306340 | 9564 | 9676 |
| 23531562 | 9565 | 9677 |
| A.3.4 Involved in secretion & adhesion | | |
| hp3e10946orf2 | 996 | 5758 |
| hp3e10946_7157843_f2_2 | 997 | 5759 |
| hp3e10946orf1 | 998 | 5760 |
| hp3e10946_2226010_f1_1 | 999 | 5761 |
| hp3e10946_7157843_f2_2 | 1000 | 5762 |
| hp4e13394_26750068_c3_113 | 1001 | 5763 |
| 197166 | 1002 | 5764 |
| 11ae80818_197166_c3_63 | 1003 | 5765 |
| 03ap21820orf13 | 1004 | 5766 |
| 06cp30603orf16 | 1005 | 5767 |
| 06cp30603_23452_c3_80 | 1006 | 5768 |
| 09cp10713_23452_c3_195 | 1007 | 5769 |
| 24417212 | 9566 | 9678 |
| A.3.5 Proteins involved in regulation | | |
| 01xe21717orf1 | 1008 | 5770 |
| 01xe21717orf8 | 1009 | 5771 |
| 01xe21717orf7 | 1010 | 5772 |
| 01xe21717orf17 | 1011 | 5773 |
| 05ep10815_204827_c2_92 | 1012 | 5774 |
| 01xe21717orf2 | 1013 | 5775 |
| 26261040 | 1014 | 5776 |
| 01xe21717orf18 | 1015 | 5777 |
| 05ep10815_21566387_c3_104 | 1016 | 5778 |
| 03ce11207_23852058_c3_270 | 1017 | 5779 |
| hpy09cp10713orf_0002.aa | 1018 | 5780 |
| hpy09cp10713orf_0005.aa | 1019 | 5781 |
| hpy09cp10713orf_0004.aa | 1020 | 5782 |
| 09cp10713orf27 | 1021 | 5783 |
| 09cp10713orf32 | 1022 | 5784 |
| 09cp10713_21915943_c3_34 | 1023 | 5785 |
| 09cp10713_21915943_c3_33 | 1024 | 5786 |
| 32422343 | 1025 | 5787 |
| 02ae11612_32422343_c2_83 | 1026 | 5788 |
| 02ae11612orf25 | 1027 | 5789 |
| A.3.6 Chaperones | | |
| 06ep10306orf12 | 1028 | 5790 |
| 24651083 | 1029 | 5791 |
| 03ae10804_13862626_c3_45 | 1030 | 5792 |
| 06ep10306orf3 | 1031 | 5793 |
| 30089217 | 9567 | 9679 |
| A.3.7 Proteins involved in cell division | | |
| 01ce11016orf13 | 1032 | 5794 |
| 11cp11224orf4 | 1033 | 5795 |
| hpy11cp11224orf_0001.aa | 1034 | 5796 |
| hp6p80503_20051588_c3_36 | 1035 | 5797 |
| 19626250 | 1036 | 5798 |
| 02ge10116_22704567_c3_33 | 1037 | 5799 |
| 02ge10116_22704567_c3_159 | 1038 | 5800 |
| 02ge20116orf22 | 1039 | 5801 |
| hp6p10723orf8 | 1040 | 5802 |
| 06ap11119_33259403_f3_17 | 1041 | 5803 |
| 12gp31106_958182_f3_39 | 1042 | 5804 |
| 13ap11517orf5 | 1043 | 5805 |
| 06gp71906_24882783_c2_156 | 1044 | 5806 |
| 12gp31106_24902212_f1_12 | 1045 | 5807 |
| 13ap11517orf9 | 1046 | 5808 |
| 04ge11713orf2 | 1047 | 5809 |
| 04ge11713orf11 | 1048 | 5810 |
| 04cp11202_956260_f3_35 | 1049 | 5811 |
| 06gp10920orf3 | 1050 | 5812 |
| hp6p10606_26851452_c1_22 | 1051 | 5813 |
| hp8e10080_26851452_c1_66 | 1052 | 5814 |
| 22704567 | 9568 | 9680 |
| 24427340 | 9569 | 9681 |
| A.3.8 Proteins involved in motility | | |
| 06ep30223_12921892_f1_35 | 1053 | 5815 |
| hp3e11024orf14 | 1054 | 5816 |
| A.3.9 Other inner membrane proteins | | |
| hpy11cp12002orf_0003.aa | 1055 | 5817 |
| hp7p10290_24223782_c2_22 | 1056 | 5818 |
| 1365943 | 1057 | 5819 |
| hp5e12982_1365943_c1_13 | 1058 | 5820 |
| hp5e12982orf14 | 1059 | 5821 |
| 02ge10116_431293_f2_45 | 1060 | 5822 |
| hpyhp5e15276orf_0004.aa | 1061 | 5823 |
| 02ge11810_67_f1_3 | 1062 | 5824 |
| 4339708 | 1063 | 5825 |
| hpy06ee10709orf_0007.aa | 1064 | 5826 |
| 06ee10709orf16 | 1065 | 5827 |
| 06ee10709_4339708_c2_15 | 1066 | 5828 |
| 05ae30220_29406392_c3_119 | 1067 | 5829 |
| 05ae20220orf125 | 1068 | 5830 |
| hpyhp3e11168orf_0013.aa | 1069 | 5831 |
| 24039587 | 1070 | 5832 |
| hp3e11168orf15 | 1071 | 5833 |
| 04gp11701_24039587_f2_12 | 1072 | 5834 |
| 1385937 | 1073 | 5835 |
| 05ep10815_25415887_c2_94 | 1074 | 5836 |
| 01xe21717orf5 | 1075 | 5837 |
| 01ae11010_33593_c2_51 | 1076 | 5838 |
| hpyhp4e14522orf_0005.aa | 1077 | 5839 |
| 05ep10815_34179577_c1_78 | 1078 | 5840 |
| 34179577 | 1079 | 5841 |
| 01xe21717orf12 | 1080 | 5842 |
| 01ae11612_6017137_f3_10 | 1081 | 5843 |
| hp6e10180_6921933_f2_2 | 1082 | 5844 |
| 29ep10720_17089217_f3_18 | 1083 | 5845 |
| 17089217 | 1084 | 5846 |
| 11ge10309orf18 | 1085 | 5847 |
| 23439633 | 1086 | 5848 |
| 04ep41903_23439633_c2_31 | 1087 | 5849 |
| 09ae11601orf14 | 1088 | 5850 |
| 05ae20220orf35 | 1089 | 5851 |
| hp5p15870_14350428_f1_1 | 1090 | 5852 |
| 05ae20220orf56 | 1091 | 5853 |
| 05ae20220orf10 | 1092 | 5854 |
| 05ae30220_14350428_f1_9 | 1093 | 5855 |
| hpy05ae10307orf_0002.aa | 1094 | 5856 |
| 05ae30220_24220937_c2_135 | 1095 | 5857 |
| 24798427 | 1096 | 5858 |
| 05ae30220_24798427_f3_52 | 1097 | 5859 |
| 05ae20220orf31 | 1098 | 5860 |
| 05ae30220_4708337_c3_116 | 1099 | 5861 |
| 4708337 | 1100 | 5862 |
| 05ae20220orf88 | 1101 | 5863 |
| 23438840 | 1102 | 5864 |
| 12ae11404_23438840_c1_10 | 1103 | 5865 |
| 12ae11404orf12 | 1104 | 5866 |
| hpy03ae10516orf_0006.aa | 1105 | 5867 |
| 33476715 | 1106 | 5868 |
| 03ae10516orf11 | 1107 | 5869 |
| 06ap30322_4726503_c2_27 | 1108 | 5870 |
| 06ap11119_218_f3_18 | 1109 | 5871 |
| hp6p10723orf2 | 1110 | 5872 |
| 06ap11119_26351567_f1_6 | 1111 | 5873 |
| 26351567 | 1112 | 5874 |
| hp6p10723orf13 | 1113 | 5875 |
| 06ap11119_4726068_f1_5 | 1114 | 5876 |
| hp6p10723orf12 | 1115 | 5877 |
| 06ap11119_4744128_c3_65 | 1116 | 5878 |
| 4744128 | 1117 | 5879 |
| hp6p10723orf43 | 1118 | 5880 |
| 06cp11118_789033_f3_11 | 1119 | 5881 |
| 01ce11513orf9 | 1120 | 5882 |
| hp6e10967_33986087_c3_25 | 1121 | 5883 |
| 33986087 | 1122 | 5884 |
| hp2p10625orf14 | 1123 | 5885 |
| 06ep11202_36562767_c3_32 | 1124 | 5886 |
| 06cp20302orf11 | 1125 | 5887 |
| 15126875 | 1126 | 5888 |
| 12gp31106_15126875_c3_68 | 1127 | 5889 |
| 13ap11517orf31 | 1128 | 5890 |
| 23473437 | 1129 | 5891 |
| 14cp10119orf14 | 1130 | 5892 |
| hpy14cp10119orf_0001.aa | 1131 | 5893 |
| 04cp11202_1953337_f3_49 | 1132 | 5894 |
| 06gp71906_26567586_f1_14 | 1133 | 5895 |
| hpy04ge11713orf_0011.aa | 1134 | 5896 |
| 2111040 | 1135 | 5897 |
| 07ae11008_1307312_c3_58 | 1136 | 5898 |
| 09cp10502orf14 | 1137 | 5899 |
| hpy09cp10502orf_0012.aa | 1138 | 5900 |
| 09cp10502orf12 | 1139 | 5901 |
| 14gp11820orf13 | 1140 | 5902 |
| hpy14gp11820orf_0011.aa | 1141 | 5903 |
| 07ae11008_14494077_c2_47 | 1142 | 5904 |
| 09cp20502_14494077_c1_34 | 1143 | 5905 |
| 23438887 | 1144 | 5906 |
| 09cp10502orf16 | 1145 | 5907 |
| 24409577 | 1146 | 5908 |
| 07ae11008_431591_c1_38 | 1147 | 5909 |
| 09cp10502orf17 | 1148 | 5910 |
| 07ae11008_10546918_f1_5 | 1149 | 5911 |
| 07ae11008_29548890_f3_20 | 1150 | 5912 |
| 09cp10502orf7 | 1151 | 5913 |
| 3953952 | 1152 | 5914 |
| 09cp20502_3953952_c1_33 | 1153 | 5915 |
| 14gp11820orf4 | 1154 | 5916 |
| 30730068 | 1155 | 5917 |
| 07ae11008_4484500_c2_52 | 1156 | 5918 |
| 09cp10502orf22 | 1157 | 5919 |
| 14ap10815_23631317_c3_27 | 1158 | 5920 |
| 23631317 | 1159 | 5921 |
| hp3e10349orf25 | 1160 | 5922 |
| 14cp20718_25812655_c3_20 | 1161 | 5923 |
| 07ap61111_24744430_c3_112 | 1162 | 5924 |
| 29zp10241orf14 | 1163 | 5925 |
| 07ap61111_9776562_c2_86 | 1164 | 5926 |
| 9776562 | 1165 | 5927 |
| 14cp20718_9776562_c1_14 | 1166 | 5928 |
| 13865928 | 1167 | 5929 |
| 14ee41924_13865928_c1_71 | 1168 | 5930 |
| 02ep30607orf31 | 1169 | 5931 |
| 14ee41924_26833437_f2_36 | 1170 | 5932 |
| 30100332 | 1171 | 5933 |
| hp2e10911_31363125_c2_92 | 1172 | 5934 |
| hp2e10911_24744076_c1_81 | 1173 | 5935 |
| hp2e10911orf30 | 1174 | 5936 |
| 07ee50709_16601067_f1_28 | 1175 | 5937 |
| hpy07ce10203orf_0013.aa | 1176 | 5938 |
| hpy07ce10203orf_0012.aa | 1177 | 5939 |
| hpy07ce10203orf_0014.aa | 1178 | 5940 |
| 07ce10203orf22 | 1179 | 5941 |
| 02ae31010_16679640_f2_21 | 1180 | 5942 |
| 23526667 | 1181 | 5943 |
| 02ae31010_24395801_f1_8 | 1182 | 5944 |
| 07ee50709_16679640_f3_60 | 1183 | 5945 |
| 207817 | 1184 | 5946 |
| 29ge30321_207817_f2_8 | 1185 | 5947 |
| 01ce11618orf10 | 1186 | 5948 |
| hp5p15641_23945317_f3_13 | 1187 | 5949 |
| 23945317 | 1188 | 5950 |
| hp5p15641orf9 | 1189 | 5951 |
| 1071890 | 1190 | 5952 |
| 02ae31010_4719050_c2_83 | 1191 | 5953 |
| 02ce11022orf7 | 1192 | 5954 |
| 598933 | 1193 | 5955 |
| hp1p13852orf5 | 1194 | 5956 |
| 06cp11217_6540927_f1_7 | 1195 | 5957 |
| 07ge11717_6540927_c1_16 | 1196 | 5958 |
| 09ce10413_33257312_c3_25 | 1197 | 5959 |
| 09cp10224_3946875_f1_2 | 1198 | 5960 |
| hp1p11244orf10 | 1199 | 5961 |
| 26758437 | 1200 | 5962 |
| 14ap11617_26758437_c2_10 | 1201 | 5963 |
| 05ap11505orf10 | 1202 | 5964 |
| 1364378 | 1203 | 5965 |
| 09ze10333_24484777_c1_14 | 1204 | 5966 |
| 03ge10505orf14 | 1205 | 5967 |
| 09cp10713_3984818_c2_28 | 1206 | 5968 |
| 13ae10610_26054808_c1_25 | 1207 | 5969 |
| 13ae10610_6097567_c3_36 | 1208 | 5970 |
| 12ee10904orf1 | 1209 | 5971 |
| hpy12ee10904orf_0001.aa | 1210 | 5972 |
| 14gp11423orf3 | 1211 | 5973 |
| 14gp11423_24407165_c2_21 | 1212 | 5974 |
| 14gp11423_35667791_c1_13 | 1213 | 5975 |
| 14gp11423orf4 | 1214 | 5976 |
| 14gp11423_19812527_c3_95 | 1215 | 5977 |
| 12694087 | 1216 | 5978 |
| 12ae10622_24004137_f3_33 | 1217 | 5979 |
| 04ep71403orf12 | 1218 | 5980 |
| 12ae10622_259665_f2_18 | 1219 | 5981 |
| 259665 | 1220 | 5982 |
| 12ae10622orf9 | 1221 | 5983 |
| 32663212 | 1222 | 5984 |
| 07ce11019_26596881_c1_12 | 1223 | 5985 |
| 07cp21714orf14 | 1224 | 5986 |
| 12ge10321_36362687_f2_11 | 1225 | 5987 |
| 06gp11920orf8 | 1226 | 5988 |
| 04ep71403orf10 | 1227 | 5989 |
| 34194093 | 1228 | 5990 |
| 50062 | 1229 | 5991 |
| 04ep71403orf15 | 1230 | 5992 |
| 12ae10622_50062_f3_32 | 1231 | 5993 |
| 24222885 | 1232 | 5994 |
| hp3e10342_24222885_c3_18 | 1233 | 5995 |
| 01ge11619_24222885_c3_16 | 1234 | 5996 |
| 11ge10309orf14 | 1235 | 5997 |
| 05ep21124_189017_f1_1 | 1236 | 5998 |
| hp3e11188_189017_c1_19 | 1237 | 5999 |
| hp4e13394_24411011_f2_27 | 1238 | 6000 |
| 24411011 | 1239 | 6001 |
| hp4e13394orf5 | 1240 | 6002 |
| 04ep10206orf5 | 1241 | 6003 |
| hp1e13852_2115918_f1_2 | 1242 | 6004 |
| 03ee11215_2150290_f3_19 | 1243 | 6005 |
| 2150290 | 1244 | 6006 |
| 03ee11215orf20 | 1245 | 6007 |
| 13704718 | 1246 | 6008 |
| hp1e13852_5861458_c1_21 | 1247 | 6009 |
| 04ep10206orf22 | 1248 | 6010 |
| hp4e53394_36131715_f1_14 | 1249 | 6011 |
| hp1e13852_4486092_f2_7 | 1250 | 6012 |
| 4486092 | 1251 | 6013 |
| 06ae11020orf2 | 1252 | 6014 |
| 06ce10808orf2 | 1253 | 6015 |
| hpy06ce10808orf_0002.aa | 1254 | 6016 |
| hp1p13939_25397327_f3_22 | 1255 | 6017 |
| 06ep30223_1206675_f1_38 | 1256 | 6018 |
| 1206675 | 1257 | 6019 |
| hp3e11024orf17 | 1258 | 6020 |
| 12617677 | 1259 | 6021 |
| 06ep30223_12617677_f3_88 | 1260 | 6022 |
| 14gp12015orf14 | 1261 | 6023 |
| hpy02ae20515orf_0001.aa | 1262 | 6024 |
| hpy02xp10215orf_0001.aa | 1263 | 6025 |
| hpy02xp10215orf_0003.aa | 1264 | 6026 |
| 07ee30411_9797156_f1_1 | 1265 | 6027 |
| 06ep30223_5078388_c2_136 | 1266 | 6028 |
| 01ce11104_11878127_c1_9 | 1267 | 6029 |
| 16ep10117orf6 | 1268 | 6030 |
| 26052137 | 1269 | 6031 |
| 02ae11612_20118768_f2_28 | 1270 | 6032 |
| 05cp11911orf15 | 1271 | 6033 |
| 21511555 | 1272 | 6034 |
| 02ae11612_21511555_f1_18 | 1273 | 6035 |
| 05cp11911orf13 | 1274 | 6036 |
| 02ae11612_23437502_f3_39 | 1275 | 6037 |
| 23437502 | 1276 | 6038 |
| 02ae11612orf14 | 1277 | 6039 |
| 01ce11104_36134661_c2_11 | 1278 | 6040 |
| 36134661 | 1279 | 6041 |
| 16ep10117orf7 | 1280 | 6042 |
| 03xe11215orf5 | 1281 | 6043 |
| 02ae11612_3933437_c3_96 | 1282 | 6044 |
| 01ce21104_3933437_c2_78 | 1283 | 6045 |
| 3933437 | 1284 | 6046 |
| hp6e10264_2534579_c1_3 | 1285 | 6047 |
| hp6e30264_24022187_c2_6 | 1286 | 6048 |
| hp5e15084orf5 | 1287 | 6049 |
| hp6e10491_17038885_f2_7 | 1288 | 6050 |
| hp6e40491_36329178_f1_1 | 1289 | 6051 |
| 35360843 | 1290 | 6052 |
| hp6p10509_21540625_f3_7 | 1291 | 6053 |
| 16ae10508orf3 | 1292 | 6054 |
| 24413512 | 1293 | 6055 |
| 12ap10605_4491302_c1_6 | 1294 | 6056 |
| 29gp10119orf7 | 1295 | 6057 |
| 20976500 | 1296 | 6058 |
| 07ep11916_20976500_f1_8 | 1297 | 6059 |
| hp6p12244_20976500_f2_21 | 1298 | 6060 |
| 01cp11414orf10 | 1299 | 6061 |
| hpy02ae11211orf_0001.aa | 1300 | 6062 |
| hp6p12244_34609662_f1_1 | 1301 | 6063 |
| 34172639 | 1302 | 6064 |
| hp6p12244_5869768_f2_24 | 1303 | 6065 |
| hp1p13922orf1 | 1304 | 6066 |
| hp6p22217_25398250_f3_16 | 1305 | 6067 |
| 25398250 | 1306 | 6068 |
| 13ee12016orf18 | 1307 | 6069 |
| 01ae11421orf4 | 1308 | 6070 |
| hpy01ae11421orf_0001.aa | 1309 | 6071 |
| 01ae11421orf3 | 1310 | 6072 |
| 13ae10712_14100018_f2_12 | 1311 | 6073 |
| 14cp10705orf4 | 1312 | 6074 |
| 13ae10712_5109376_f3_18 | 1313 | 6075 |
| 06ge20501_14100018_c1_34 | 1314 | 6076 |
| 11ae12004_14100018_c1_32 | 1315 | 6077 |
| 4728193 | 1316 | 6078 |
| 02gp20814_4728193_f1_3 | 1317 | 6079 |
| 14ee10308orf9 | 1318 | 6080 |
| hpy07ge20415orf_0019.aa | 1319 | 6081 |
| 10745275 | 1320 | 6082 |
| 07ge20415orf30 | 1321 | 6083 |
| hp7e10429_22141052_c3_41 | 1322 | 6084 |
| 5993958 | 1323 | 6085 |
| hp7e10429_24023462_c2_36 | 1324 | 6086 |
| 07ge20415orf39 | 1325 | 6087 |
| hp7p90421_23477042_c2_43 | 1326 | 6088 |
| 33218912 | 1327 | 6089 |
| hp3p10349orf32 | 1328 | 6090 |
| 02ce41018_33367812_f2_14 | 1329 | 6091 |
| 24078837 | 1330 | 6092 |
| 07ap20216_24078837_f1_4 | 1331 | 6093 |
| 05ap21216orf7 | 1332 | 6094 |
| hp1p14013orf16 | 1333 | 6095 |
| 02cp10615orf1 | 1334 | 6096 |
| hp1p14013orf13 | 1335 | 6097 |
| hpyhp1p14013orf_0015.aa | 1336 | 6098 |
| hp1p14013_21507007_c2_19 | 1337 | 6099 |
| hp8e10080_16267837_c3_92 | 1338 | 6100 |
| 02cp10615_6515756_f1_1 | 1339 | 6101 |
| hpyhp1p14013orf_0013.aa | 1340 | 6102 |
| 663530 | 1341 | 6103 |
| hp1p14013orf4 | 1342 | 6104 |
| hp1p14013_4556713_f3_11 | 1343 | 6105 |
| 03gp20123orf2 | 1344 | 6106 |
| hpy03gp20123orf_0002.aa | 1345 | 6107 |
| hpyhp4p12360orf_0001.aa | 1346 | 6108 |
| 09cp61003_13710936_c1_73 | 1347 | 6109 |
| hp7p10290_24226550_f1_4 | 1348 | 6110 |
| hp3p10156orf11 | 1349 | 6111 |
| hp5e11634orf2 | 1350 | 6112 |
| 03ep12017orf1 | 1351 | 6113 |
| 11cp12002orf7 | 1352 | 6114 |
| hp7p10290_244843_c1_16 | 1353 | 6115 |
| 12ge10305orf3 | 1354 | 6116 |
| 12ge10305orf4 | 1355 | 6117 |
| hp6e12267_32424058_f2_22 | 1356 | 6118 |
| hp2p10272orf6 | 1357 | 6119 |
| hp2p10272orf3 | 1358 | 6120 |
| hp2p10272_23880380_f2_11 | 1359 | 6121 |
| 06ge10115orf10 | 1360 | 6122 |
| hpy06ge10115orf_0011.aa | 1361 | 6123 |
| 02gp20706_14494561_c1_37 | 1362 | 6124 |
| 02gp20706_23535062_f1_2 | 1363 | 6125 |
| 01cp11710orf7 | 1364 | 6126 |
| 02gp20706_5314192_f2_11 | 1365 | 6127 |
| 01cp11710orf12 | 1366 | 6128 |
| 12969218 | 1367 | 6129 |
| 02ge11622_23494043_f1_6 | 1368 | 6130 |
| 13ee10216orf5 | 1369 | 6131 |
| hpyhp2e11875orf_0004.aa | 1370 | 6132 |
| 06gp11209orf3 | 1371 | 6133 |
| hpy06gp11209orf_0001.aa | 1372 | 6134 |
| hpy01ce10320orf_0009.aa | 1373 | 6135 |
| 01ce10320_10742802_f1_5 | 1374 | 6136 |
| 01cp20708_19542968_c1_46 | 1375 | 6137 |
| 14ee10419orf19 | 1376 | 6138 |
| hpy06ep10822orf_0002.aa | 1377 | 6139 |
| 06ep10822orf1 | 1378 | 6140 |
| hpy06ep10822orf_0001.aa | 1379 | 6141 |
| hp2e11875orf4 | 1380 | 6142 |
| 01ae11010_29308578_c1_38 | 1381 | 6143 |
| 14ce31519_1299140_f1_4 | 1382 | 6144 |
| 02ce10809orf1 | 1383 | 6145 |
| 04ep41903_23437543_c3_40 | 1384 | 6146 |
| 02cp10815orf1 | 1385 | 6147 |
| 09ae11601orf2 | 1386 | 6148 |
| 04ep41903_5351512_f2_13 | 1387 | 6149 |
| 23867207 | 1388 | 6150 |
| hp5p15212_13095752_c3_36 | 1389 | 6151 |
| 02ce10213orf23 | 1390 | 6152 |
| hpy07ee20513orf_0004.aa | 1391 | 6153 |
| 07ee20513orf1 | 1392 | 6154 |
| 07ee20513orf12 | 1393 | 6155 |
| 06ae11016_15634702_f2_7 | 1394 | 6156 |
| 09ae10512_15634702_f3_15 | 1395 | 6157 |
| 07ee20513orf10 | 1396 | 6158 |
| 09ae10512_6929702_f1_5 | 1397 | 6159 |
| 06ae11016_6929702_f3_27 | 1398 | 6160 |
| 13ee10424orf2 | 1399 | 6161 |
| 06ap10305orf2 | 1400 | 6162 |
| 06ep10615_1230462_c3_105 | 1401 | 6163 |
| 13ee10424_1230462_f3_4 | 1402 | 6164 |
| hpy07ge11107orf_0001.aa | 1403 | 6165 |
| 11ee11718orf1 | 1404 | 6166 |
| 06ep10615_16226577_c1_67 | 1405 | 6167 |
| 06gp10409_33337812_f2_10 | 1406 | 6168 |
| 04ge11613orf2 | 1407 | 6169 |
| 06gp10409_5204827_f3_15 | 1408 | 6170 |
| 06gp10409orf4 | 1409 | 6171 |
| 04cp11202_19532827_c3_111 | 1410 | 6172 |
| 04ge11713orf38 | 1411 | 6173 |
| 04cp11202_29938885_f3_33 | 1412 | 6174 |
| 04cp11202orf3 | 1413 | 6175 |
| hpy04cp11202orf_0001.aa | 1414 | 6176 |
| 06gp71906_24352311_c3_199 | 1415 | 6177 |
| hpy04cp11202orf_0003.aa | 1416 | 6178 |
| 04ge11713orf16 | 1417 | 6179 |
| hpy04ge11713orf_0001.aa | 1418 | 6180 |
| 04cp11202orf1 | 1419 | 6181 |
| 04cp11202_25394506_f2_14 | 1420 | 6182 |
| hpy02cp11822orf_0010.aa | 1421 | 6183 |
| 02cp11822orf4 | 1422 | 6184 |
| 02cp11822orf3 | 1423 | 6185 |
| 01ae12021orf1 | 1424 | 6186 |
| hpy01ae12021orf_0001.aa | 1425 | 6187 |
| 02cp11822orf8 | 1426 | 6188 |
| 04cp11202_4899192_f2_29 | 1427 | 6189 |
| 06gp71906_4899192_c3_210 | 1428 | 6190 |
| 04cp11202_4899192_f3_44 | 1429 | 6191 |
| hp5p15861_12698442_f1_3 | 1430 | 6192 |
| 02cp20821orf12 | 1431 | 6193 |
| hp5p15861_14557666_f3_13 | 1432 | 6194 |
| 02cp20821orf9 | 1433 | 6195 |
| 14ee41924_12698442_f2_35 | 1434 | 6196 |
| hp2e10911orf46 | 1435 | 6197 |
| 07ap11213_21534415_c3_34 | 1436 | 6198 |
| 01ce11618orf19 | 1437 | 6199 |
| 11ae10212orf4 | 1438 | 6200 |
| 29ge30321_34569437_f2_9 | 1439 | 6201 |
| hp2e10911_34569437_c2_85 | 1440 | 6202 |
| 29ge30321_470158_f1_2 | 1441 | 6203 |
| 01ce11618orf23 | 1442 | 6204 |
| 11ep11915orf1 | 1443 | 6205 |
| hpy11ep11915orf_0001.aa | 1444 | 6206 |
| hp6p10904_1297062_c3_24 | 1445 | 6207 |
| 01ee20804orf3 | 1446 | 6208 |
| 09cp10224_25394752_c1_42 | 1447 | 6209 |
| 09cp10405orf4 | 1448 | 6210 |
| hp4e13394_6854542_c3_114 | 1449 | 6211 |
| hp1e13852_6285437_c3_39 | 1450 | 6212 |
| 04ep10206orf16 | 1451 | 6213 |
| hp1e13852_2915903_c1_22 | 1452 | 6214 |
| 04ep10206orf23 | 1453 | 6215 |
| hp4e53394_2915903_f2_43 | 1454 | 6216 |
| 20173437 | 1455 | 6217 |
| 06ep30223_20173437_f1_37 | 1456 | 6218 |
| hp3e11024orf16 | 1457 | 6219 |
| 06ce11002_25391000_f1_2 | 1458 | 6220 |
| 06ce11002orf5 | 1459 | 6221 |
| 06ep30223_26444203_c1_118 | 1460 | 6222 |
| 07gp10713orf1 | 1461 | 6223 |
| 06ep30223_34180312_f2_65 | 1462 | 6224 |
| hp3e11024orf10 | 1463 | 6225 |
| 06ep30223_3964062_f3_96 | 1464 | 6226 |
| hp3e11024orf7 | 1465 | 6227 |
| 06ce11002orf6 | 1466 | 6228 |
| 06ce11002orf9 | 1467 | 6229 |
| 06ce11002orf4 | 1468 | 6230 |
| 06ce11002_4461718_f1_3 | 1469 | 6231 |
| 06ep30223_5078517_f3_89 | 1470 | 6232 |
| 14gp12015orf15 | 1471 | 6233 |
| 29ae22003orf5 | 1472 | 6234 |
| hpy29ae22003orf_0001.aa | 1473 | 6235 |
| 01cp31002_19539202_f3_8 | 1474 | 6236 |
| hpyhp4e14155orf_0004.aa | 1475 | 6237 |
| hpyhp4e14155orf_0003.aa | 1476 | 6238 |
| 06ap11008orf1 | 1477 | 6239 |
| hpy06ap11008orf_0001.aa | 1478 | 6240 |
| hp6p10590_22290637_f2_11 | 1479 | 6241 |
| 12ap10605_22459405_f2_2 | 1480 | 6242 |
| 29gp10119orf2 | 1481 | 6243 |
| hp3e10302orf25 | 1482 | 6244 |
| hp6p12129_2847212_f1_1 | 1483 | 6245 |
| 02ae11211orf7 | 1484 | 6246 |
| 02ae11211orf2 | 1485 | 6247 |
| 02ae11211orf10 | 1486 | 6248 |
| hp6p12244_3987580_f2_17 | 1487 | 6249 |
| 02ce11418orf2 | 1488 | 6250 |
| 01cp11414orf14 | 1489 | 6251 |
| hp6p12244_5913592_f1_7 | 1490 | 6252 |
| hpy29ge10111orf_0003.aa | 1491 | 6253 |
| 29ge10111orf9 | 1492 | 6254 |
| 06ce20610_26448380_c2_31 | 1493 | 6255 |
| 12ap11614_24400293_c2_10 | 1494 | 6256 |
| 12ap11614orf11 | 1495 | 6257 |
| hp7e10429_25525302_c2_37 | 1496 | 6258 |
| 07ge20415orf42 | 1497 | 6259 |
| 03ap21820orf15 | 1498 | 6260 |
| 03ap21820orf14 | 1499 | 6261 |
| 03ap21820orf1 | 1500 | 6262 |
| 11ae80818_421888_c2_52 | 1501 | 6263 |
| 02ep20506_23712750_f2_7 | 1502 | 6264 |
| 01ce11513orf6 | 1503 | 6265 |
| hpyhp5e15440orf_0015.aa | 1504 | 6266 |
| hp3p10343orf5 | 1505 | 6267 |
| 14ee11217orf5 | 1506 | 6268 |
| 14ee11217orf1 | 1507 | 6269 |
| 11ap20714_33992032_c1_62 | 1508 | 6270 |
| 06ap11418_33992032_f2_10 | 1509 | 6271 |
| hp1p14013orf1 | 1510 | 6272 |
| hp1p14013_24239055_f3_8 | 1511 | 6273 |
| hp1p14013orf21 | 1512 | 6274 |
| hp1p14013orf15 | 1513 | 6275 |
| hp1p14013_34187518_c3_24 | 1514 | 6276 |
| 4714375 | 9570 | 9682 |
| 29302003 | 9571 | 9683 |
| 4726503 | 9572 | 9684 |
| 40339452 | 9573 | 9685 |
| 3242337 | 9574 | 9686 |
| 3385833 | 9575 | 9687 |
| 19536458 | 9576 | 9688 |
| 34097707 | 9577 | 9689 |
| 3360130 | 9578 | 9690 |
| 2548562 | 9579 | 9691 |
| 20023400 | 9580 | 9692 |
| 29531590 | 9581 | 9693 |
| 23494043 | 9582 | 9694 |
| 36520792 | 9583 | 9695 |
| 34109763 | 9584 | 9696 |
| 907827 | 9585 | 9697 |
| 12698442 | 9586 | 9698 |
| 55843 | 9587 | 9699 |
| 25976418 | 9588 | 9700 |
| 34573431 | 9589 | 9701 |
| 3987580 | 9590 | 9702 |
| 33595708 | 9591 | 9703 |
| A.4 Other cell envelope proteins | | |
| hpy12ge20305orf_0013.aa | 1515 | 6277 |
| 01ce61016_16619192_c3_161 | 1516 | 6278 |
| 09cp61003_16619192_c2_83 | 1517 | 6279 |
| 01ce61016_1056562_c3_123 | 1518 | 6280 |
| hpy06ge10115orf_0008.aa | 1519 | 6281 |
| 02gp20706_1050787_c3_60 | 1520 | 6282 |
| 06ge10115orf8 | 1521 | 6283 |
| 02ge10116_15632000_c2_114 | 1522 | 6284 |
| 23610905 | 1523 | 6285 |
| 05ep10815_1222937_f2_39 | 1524 | 6286 |
| 01xe21717orf40 | 1525 | 6287 |
| 20415937 | 1526 | 6288 |
| 04cp11202_20415937_f2_25 | 1527 | 6289 |
| 01ae12021orf7 | 1528 | 6290 |
| 04ge11713orf28 | 1529 | 6291 |
| 04ge11713orf35 | 1530 | 6292 |
| 04ge11713orf36 | 1531 | 6293 |
| 04cp11202_24256567_c3_117 | 1532 | 6294 |
| hp5e15044_4554652_f3_3 | 1533 | 6295 |
| hpyhp1p13868orf_0008.aa | 1534 | 6296 |
| 07cp10312orf5 | 1535 | 6297 |
| hp6p10233_12273302_f1_1 | 1536 | 6298 |
| 07ce10312_4554652_f3_2 | 1537 | 6299 |
| 04ae61517_21744091_f3_5 | 1538 | 6300 |
| hpy13ap11420orf_0001.aa | 1539 | 6301 |
| 04ae61517_12345837_f1_2 | 1540 | 6302 |
| 04ae61517_12345837_f2_4 | 1541 | 6303 |
| 11cp12006orf10 | 1542 | 6304 |
| 11cp12006orf13 | 1543 | 6305 |
| hp2e10911_24855312_c1_69 | 1544 | 6306 |
| 01ce11618orf24 | 1545 | 6307 |
| hpy01ce11618orf_0014.aa | 1546 | 6308 |
| 29ge30321_34157812_f3_10 | 1547 | 6309 |
| hpy01ce11618orf_0008.aa | 1548 | 6310 |
| 29ge30321_12913562_f1_1 | 1549 | 6311 |
| 29ge30321_135253_f2_6 | 1550 | 6312 |
| 01ce11618orf22 | 1551 | 6313 |
| 01ce11618orf7 | 1552 | 6314 |
| 01ce11618orf15 | 1553 | 6315 |
| hp2e10911_3349_c1_63 | 1554 | 6316 |
| 02ae31010_4818967_f3_41 | 1555 | 6317 |
| 01ce11618orf5 | 1556 | 6318 |
| 02ae31010_15652187_f1_17 | 1557 | 6319 |
| 07ee50709_4818967_f2_43 | 1558 | 6320 |
| 04ap12016_25501501_f1_1 | 1559 | 6321 |
| 29ep10720_25501501_c2_33 | 1560 | 6322 |
| 25501501 | 1561 | 6323 |
| 11ge10309orf63 | 1562 | 6324 |
| 02ae11612orf23 | 1563 | 6325 |
| 02ae11612_31453292_c3_97 | 1564 | 6326 |
| 02ae11612_6536567_c2_81 | 1565 | 6327 |
| hp5e25328_6539692_c3_148 | 1566 | 6328 |
| 02ae11612orf31 | 1567 | 6329 |
| hp5p15575_1053590_c1_35 | 1568 | 6330 |
| hpyhp5p15575orf_0005.aa | 1569 | 6331 |
| hp5p15575_29300311_c1_29 | 1570 | 6332 |
| 14ce10720orf3 | 1571 | 6333 |
| hp7e10433_5345837_c3_13 | 1572 | 6334 |
| hp7e10433_25520337_c2_8 | 1573 | 6335 |
| hpy01ee21118orf_0002.aa | 1574 | 6336 |
| hp7e10433_5345837_c2_8 | 1575 | 6337 |
| 24256572 | 9592 | 9704 |
| B. CYTOPLASMIC PROTEINS | | |
| B.1 Proteins involved in energy metabolism | | |
| 09ap20802orf36 | 1576 | 6338 |
| hp2p10272_11885836_c1_27 | 1577 | 6339 |
| hp6p21623_16491412_f1_1 | 1578 | 6340 |
| 07gp11909_19665932_c3_16 | 1579 | 6341 |
| 07gp11909orf3 | 1580 | 6342 |
| 02ap11117_19665932_c2_62 | 1581 | 6343 |
| hp2p10272_23835927_f2_13 | 1582 | 6344 |
| 09ap20802orf16 | 1583 | 6345 |
| 07gp11909orf1 | 1584 | 6346 |
| 07gp11909_993792_f3_8 | 1585 | 6347 |
| 09ap20802orf15 | 1586 | 6348 |
| 09ap20802orf7 | 1587 | 6349 |
| 09ap20802orf6 | 1588 | 6350 |
| hp2p10272_26366562_f1_3 | 1589 | 6351 |
| 02ge11622_24245875_f3_44 | 1590 | 6352 |
| 13ee10216orf12 | 1591 | 6353 |
| 4897177 | 1592 | 6354 |
| 02ge11622_36126312_c1_53 | 1593 | 6355 |
| 13ee10216orf56 | 1594 | 6356 |
| 13ee10216orf14 | 1595 | 6357 |
| hpy04ep11803orf_0001.aa | 1596 | 6358 |
| 04ep11803orf3 | 1597 | 6359 |
| 02ge11622_4101568_f3_46 | 1598 | 6360 |
| 06ee10709_10270878_f1_1 | 1599 | 6361 |
| 06ee10709orf1 | 1600 | 6362 |
| hpy06ee10709orf_0001.aa | 1601 | 6363 |
| 02ge41622_4102312_f1_17 | 1602 | 6364 |
| 02ge41622_24803341_f2_38 | 1603 | 6365 |
| 02ge11622_5271943_f3_45 | 1604 | 6366 |
| 13ee10216orf13 | 1605 | 6367 |
| hpy13ee10216orf_0015.aa | 1606 | 6368 |
| 13ee10216orf74 | 1607 | 6369 |
| 13ee10216orf85 | 1608 | 6370 |
| 02ge11622_6301_c1_55 | 1609 | 6371 |
| 02ge41622_6301_c2_71 | 1610 | 6372 |
| 01ae11010_24220625_f2_13 | 1611 | 6373 |
| 01ce10320orf1 | 1612 | 6374 |
| hpy01ce10320orf_0004.aa | 1613 | 6375 |
| 01ce10320orf2 | 1614 | 6376 |
| 01ce10320_24641662_f2_6 | 1615 | 6377 |
| 01ce10320_35673442_f1_2 | 1616 | 6378 |
| 01ce10320orf8 | 1617 | 6379 |
| 06ae11405orf11 | 1618 | 6380 |
| hpy06ae11405orf_0010.aa | 1619 | 6381 |
| 02ap10801orf1 | 1620 | 6382 |
| 05ep10815_26679687_c1_73 | 1621 | 6383 |
| 03ce11207_5351377_c3_267 | 1622 | 6384 |
| 09ae11601orf13 | 1623 | 6385 |
| 04ep41903_3251550_c3_39 | 1624 | 6386 |
| 05ae30220_26351674_c1_64 | 1625 | 6387 |
| hpy05ae20220orf_0043.aa | 1626 | 6388 |
| hp2e10626orf2 | 1627 | 6389 |
| hpyhp2e10626orf_0002.aa | 1628 | 6390 |
| 05ae30220_22861038_c2_136 | 1629 | 6391 |
| 05ae30220_10977343_c1_140 | 1630 | 6392 |
| hp5p15861_1443817_f3_8 | 1631 | 6393 |
| 02cp20821orf4 | 1632 | 6394 |
| 14ee41924_6819555_f1_10 | 1633 | 6395 |
| 11ce10917orf4 | 1634 | 6396 |
| 11ce10917orf5 | 1635 | 6397 |
| 11ce10917orf1 | 1636 | 6398 |
| 11ce10917orf7 | 1637 | 6399 |
| 14ee41924_4507757_f3_43 | 1638 | 6400 |
| 07ee11402_6819555_f1_4 | 1639 | 6401 |
| 07ap11213_10628313_c2_29 | 1640 | 6402 |
| hp2e10911orf26 | 1641 | 6403 |
| 02ae31010_6651712_f3_40 | 1642 | 6404 |
| 01ce11618orf4 | 1643 | 6405 |
| hpy09ce10413orf_0001.aa | 1644 | 6406 |
| 02ce10216orf11 | 1645 | 6407 |
| 02ce10216orf13 | 1646 | 6408 |
| 09ce10413_4875018_c1_19 | 1647 | 6409 |
| hp1p11244orf6 | 1648 | 6410 |
| hp1p11244orf9 | 1649 | 6411 |
| hp1p11244orf7 | 1650 | 6412 |
| 09cp10224_24823437_c2_49 | 1651 | 6413 |
| hpy02ep20220orf_0002.aa | 1652 | 6414 |
| 12ae10622_20322877_f2_25 | 1653 | 6415 |
| hpy05gp11901orf_0010.aa | 1654 | 6416 |
| 05gp11901orf17 | 1655 | 6417 |
| hpy05gp11901orf_0011.aa | 1656 | 6418 |
| 04ee11506orf1 | 1657 | 6419 |
| hpy04ee11506orf_0001.aa | 1658 | 6420 |
| 05ge20108_25945418_c1_6 | 1659 | 6421 |
| 11ee11408_24650879_c2_40 | 1660 | 6422 |
| hp1e80523_24650879_c1_36 | 1661 | 6423 |
| hp4e13394_3391637_c1_70 | 1662 | 6424 |
| 11ce11603orf18 | 1663 | 6425 |
| hpy11ce11603orf_0001.aa | 1664 | 6426 |
| hp4e13394_6125462_c3_116 | 1665 | 6427 |
| hpy04ep10206orf_0001.aa | 1666 | 6428 |
| 04ep10206orf14 | 1667 | 6429 |
| hp1e13852_1178175_c2_31 | 1668 | 6430 |
| hp4e53394_1178175_f1_14 | 1669 | 6431 |
| hpy03ee11215orf_0013.aa | 1670 | 6432 |
| 03ee11215orf26 | 1671 | 6433 |
| hpy03ee11215orf_0011.aa | 1672 | 6434 |
| 03ee11215orf25 | 1673 | 6435 |
| 03ee11215_6494193_c2_27 | 1674 | 6436 |
| 06ap12018orf2 | 1675 | 6437 |
| hp3e11024orf35 | 1676 | 6438 |
| 06ep30223_2079552_c2_131 | 1677 | 6439 |
| 06ce11002orf1 | 1678 | 6440 |
| hpy06ce11002orf_0001.aa | 1679 | 6441 |
| 06ce11002_113327_f1_1 | 1680 | 6442 |
| 06ep30223_23442842_c1_103 | 1681 | 6443 |
| 06ep30223_24613567_c3_150 | 1682 | 6444 |
| hpy06ap12018orf_0001.aa | 1683 | 6445 |
| 34099062 | 1684 | 6446 |
| 06ce11002orf2 | 1685 | 6447 |
| 06ce11002_34099062_f3_5 | 1686 | 6448 |
| 01ce21104_203142_c3_86 | 1687 | 6449 |
| 11ee10118orf2 | 1688 | 6450 |
| hpy11ee10118orf_0001.aa | 1689 | 6451 |
| hp5e25328_14261087_c1_80 | 1690 | 6452 |
| 11ge10308_5256_f2_1 | 1691 | 6453 |
| 11ge10308orf1 | 1692 | 6454 |
| hpy11ge10308orf_0001.aa | 1693 | 6455 |
| hp6p12158_21698387_f3_1 | 1694 | 6456 |
| hp7e10557_21698387_f1_1 | 1695 | 6457 |
| 02ae11612_600280_f3_37 | 1696 | 6458 |
| 02ae11612orf12 | 1697 | 6459 |
| hpy02ae11612orf_0001.aa | 1698 | 6460 |
| hp5e25328_34250033_f2_26 | 1699 | 6461 |
| 01ce21104_24274137_f1_1 | 1700 | 6462 |
| 03ee11115_205166_f3_2 | 1701 | 6463 |
| 03ee11115_25525876_f1_1 | 1702 | 6464 |
| 03ee11115orf1 | 1703 | 6465 |
| hpy03ee11115orf_0001.aa | 1704 | 6466 |
| 03ee11115orf2 | 1705 | 6467 |
| hp5e25328_4884687_c3_139 | 1706 | 6468 |
| hp5p15575_5861593_c1_34 | 1707 | 6469 |
| hp5p15575_5861593_c2_45 | 1708 | 6470 |
| hp6p10590_34063916_f1_4 | 1709 | 6471 |
| 02gp20814_5350312_f3_8 | 1710 | 6472 |
| 14ee10308orf6 | 1711 | 6473 |
| 07ee11905orf1 | 1712 | 6474 |
| hpy07ee11905orf_0003.aa | 1713 | 6475 |
| 14ce61516_4303467_f1_4 | 1714 | 6476 |
| hp3p10343orf1 | 1715 | 6477 |
| hpyhp3p10343orf_0002.aa | 1716 | 6478 |
| 11ap20714_33375062_f1_3 | 1717 | 6479 |
| hpy13ep12003orf_0005.aa | 1718 | 6480 |
| 13ep12003orf18 | 1719 | 6481 |
| hp6p10606_24237937_c2_27 | 1720 | 6482 |
| hp1p14013_36126562_f1_1 | 1721 | 6483 |
| hp1p14013orf5 | 1722 | 6484 |
| hp1p14013_3257800_f2_4 | 1723 | 6485 |
| hp1p14013orf10 | 1724 | 6486 |
| 02cp10615_36126562_c3_70 | 1725 | 6487 |
| 29500075 | 9593 | 9705 |
| 10737627 | 9594 | 9706 |
| B.2 Proteins involved in amino acid | | |
| metabolism | | |
| 6696887 | 1726 | 6488 |
| 02ge10116_6696887_f2_46 | 1727 | 6489 |
| 11ae10305orf4 | 1728 | 6490 |
| 02ge11622_30105343_f2_15 | 1729 | 6491 |
| 13ee10216orf38 | 1730 | 6492 |
| 02ge41622_30105343_f2_15 | 1731 | 6493 |
| 31681556 | 1732 | 6494 |
| 11gp20904_26212885_c3_33 | 1733 | 6495 |
| 11gp10904orf27 | 1734 | 6496 |
| hpyhp4e14522orf_0001.aa | 1735 | 6497 |
| 01ae11010_26344041_c1_44 | 1736 | 6498 |
| 01ae11010_26353437_c3_57 | 1737 | 6499 |
| hp4e14522orf18 | 1738 | 6500 |
| 01ae11010_788312_c1_43 | 1739 | 6501 |
| 07cp10312orf19 | 1740 | 6502 |
| 01ae11010_26353437_c2_28 | 1741 | 6503 |
| 01xe21717orf13 | 1742 | 6504 |
| 01xe21717orf21 | 1743 | 6505 |
| 05ep10815_3228467_c1_81 | 1744 | 6506 |
| 01cp20708_4568818_f2_12 | 1745 | 6507 |
| 14ee10419orf6 | 1746 | 6508 |
| 07ge11504orf9 | 1747 | 6509 |
| hpy07ge11504orf_0007.aa | 1748 | 6510 |
| 12ge10610orf1 | 1749 | 6511 |
| hpy12ge10610orf_0001.aa | 1750 | 6512 |
| 05ep10815_4864693_c2_98 | 1751 | 6513 |
| hpyhp3p10018orf_0001.aa | 1752 | 6514 |
| 09ge10522orf2 | 1753 | 6515 |
| hpy09ge10522orf_0002.aa | 1754 | 6516 |
| 05ep10815_4901462_f2_35 | 1755 | 6517 |
| hpy11ap11819orf_0001.aa | 1756 | 6518 |
| 04ep41903_36225883_f2_11 | 1757 | 6519 |
| 05ae20220orf21 | 1758 | 6520 |
| 05ae20220orf47 | 1759 | 6521 |
| 05ae20220orf65 | 1760 | 6522 |
| 05ae30220_4698842_f2_42 | 1761 | 6523 |
| hpy07ee20513orf_0015.aa | 1762 | 6524 |
| 07ee20513orf9 | 1763 | 6525 |
| 09ae10512_4336568_f3_24 | 1764 | 6526 |
| 06ep11202_4884677_c1_17 | 1765 | 6527 |
| 06cp20302orf5 | 1766 | 6528 |
| hp3e11122orf4 | 1767 | 6529 |
| 06gp31906orf3 | 1768 | 6530 |
| 06gp31906orf4 | 1769 | 6531 |
| hpy06gp31906orf_0002.aa | 1770 | 6532 |
| 06gp71906_31290937_c2_163 | 1771 | 6533 |
| 06gp71906_37592_f1_27 | 1772 | 6534 |
| hp4p11393orf3 | 1773 | 6535 |
| hpyhp4p11393orf_0001.aa | 1774 | 6536 |
| hp4p11393orf4 | 1775 | 6537 |
| 06gp71906_3986502_f3_111 | 1776 | 6538 |
| 14gp11820orf20 | 1777 | 6539 |
| 14gp11820orf27 | 1778 | 6540 |
| 09cp20502_23475342_f2_18 | 1779 | 6541 |
| 23475342 | 1780 | 6542 |
| 14gp11820orf7 | 1781 | 6543 |
| 14gp11820orf3 | 1782 | 6544 |
| 09cp20502_24613337_c2_43 | 1783 | 6545 |
| 07ae11008_24613337_c3_55 | 1784 | 6546 |
| 34189716 | 1785 | 6547 |
| 29ge30321_34189716_f3_12 | 1786 | 6548 |
| 01ce11618orf18 | 1787 | 6549 |
| 09ce52017_4005267_c1_20 | 1788 | 6550 |
| 01ep30520orf23 | 1789 | 6551 |
| 24409641 | 1790 | 6552 |
| 06cp30603_23567890_c1_86 | 1791 | 6553 |
| 05cp20518orf61 | 1792 | 6554 |
| 05cp20518orf12 | 1793 | 6555 |
| 05cp20518orf32 | 1794 | 6556 |
| 06cp30603_23595967_f2_33 | 1795 | 6557 |
| 11ge61517_23595967_f1_20 | 1796 | 6558 |
| 06cp30603_5364452_c3_141 | 1797 | 6559 |
| 04gp11213orf49 | 1798 | 6560 |
| hp4e13394orf3 | 1799 | 6561 |
| hp4e13394orf7 | 1800 | 6562 |
| hp4e13394_1296888_f2_28 | 1801 | 6563 |
| 09ap11406orf27 | 1802 | 6564 |
| 09ap11406orf17 | 1803 | 6565 |
| hp4e13394_23470963_f2_29 | 1804 | 6566 |
| hp4e13394_24636562_f2_44 | 1805 | 6567 |
| 06ep11108orf15 | 1806 | 6568 |
| 06ce10907orf2 | 1807 | 6569 |
| 09gp10903orf3 | 1808 | 6570 |
| hpy09gp10903orf_0004.aa | 1809 | 6571 |
| hp4e13394_25563962_f3_47 | 1810 | 6572 |
| 06ce10907orf1 | 1811 | 6573 |
| hpy06ce10907orf_0001.aa | 1812 | 6574 |
| hp4e13394_26736040_f1_2 | 1813 | 6575 |
| 09gp10903orf2 | 1814 | 6576 |
| 09ap11406orf26 | 1815 | 6577 |
| 09gp10903orf5 | 1816 | 6578 |
| hp4e13394_33306533_f1_3 | 1817 | 6579 |
| 09ap11406orf19 | 1818 | 6580 |
| hp4e13394_34171916_f1_4 | 1819 | 6581 |
| 04ep10206orf13 | 1820 | 6582 |
| hp1e13852_23477192_c2_30 | 1821 | 6583 |
| hp4e53394_23477192_f1_13 | 1822 | 6584 |
| 12ge10513orf2 | 1823 | 6585 |
| hpy12ge10513orf_0001.aa | 1824 | 6586 |
| 03ee11215_7320937_f1_8 | 1825 | 6587 |
| 03ee11215orf11 | 1826 | 6588 |
| hp4e53394_7320937_f1_11 | 1827 | 6589 |
| 14cp11121orf1 | 1828 | 6590 |
| 14cp11121orf7 | 1829 | 6591 |
| 05ce10208_36128141_f3_13 | 1830 | 6592 |
| 02ae11612_16834692_f1_15 | 1831 | 6593 |
| 05cp11911orf10 | 1832 | 6594 |
| hp4e14535orf7 | 1833 | 6595 |
| hp4e14535orf8 | 1834 | 6596 |
| hp4e14535orf3 | 1835 | 6597 |
| 06ep11917_24803153_c3_24 | 1836 | 6598 |
| 4570262 | 1837 | 6599 |
| 02ae11612_4570262_f3_38 | 1838 | 6600 |
| 02ae11612orf13 | 1839 | 6601 |
| 14ce10720orf4 | 1840 | 6602 |
| 14ce10720orf1 | 1841 | 6603 |
| hp5p15575_34156437_c2_36 | 1842 | 6604 |
| 05ge11422orf2 | 1843 | 6605 |
| 05ge11422orf1 | 1844 | 6606 |
| hpy05ge11422orf_0001.aa | 1845 | 6607 |
| 06ce20610_12925201_c3_35 | 1846 | 6608 |
| hp7e10228_4892592_f1_1 | 1847 | 6609 |
| hpy14ep11807orf_0001.aa | 1848 | 6610 |
| 45914063 | 1849 | 6611 |
| 12ap11614orf4 | 1850 | 6612 |
| hpy12ap11614orf_0010.aa | 1851 | 6613 |
| 12ap11614_34469527_f2_3 | 1852 | 6614 |
| 09cp21607_989055_c1_9 | 1853 | 6615 |
| 07gp31516orf6 | 1854 | 6616 |
| 11ae10710orf1 | 1855 | 6617 |
| hpy11ae10710orf_0001.aa | 1856 | 6618 |
| hp7p10287_24666030_f2_5 | 1857 | 6619 |
| hpyhp3p10349orf_0009.aa | 1858 | 6620 |
| 02ce41018_24303276_f3_19 | 1859 | 6621 |
| hp3p10349orf23 | 1860 | 6622 |
| hp7e10420_24484767_f3_18 | 1861 | 6623 |
| 06ap20306_22269807_c2_17 | 1862 | 6624 |
| 04gp11803orf6 | 1863 | 6625 |
| 03ae11503_4883587_c1_15 | 1864 | 6626 |
| 11ap20714_4883587_f3_47 | 1865 | 6627 |
| 03ae11503orf7 | 1866 | 6628 |
| 02cp11721orf11 | 1867 | 6629 |
| hp7e10100_3254805_c3_19 | 1868 | 6630 |
| 21976637 | 9595 | 9707 |
| 677088 | 9596 | 9708 |
| B.3 Proteins involved in cofactor | | |
| metabolism | | |
| hp6e12267_1204050_c2_46 | 1869 | 6631 |
| 12ge10305orf14 | 1870 | 6632 |
| hp6e12267_24089437_c3_61 | 1871 | 6633 |
| 24089437 | 1872 | 6634 |
| 12ge20305orf14 | 1873 | 6635 |
| 09ae10323orf2 | 1874 | 6636 |
| 09ae10323orf4 | 1875 | 6637 |
| 09cp61003_283442_f1_19 | 1876 | 6638 |
| 12ge20305orf28 | 1877 | 6639 |
| hp6e12267_4298467_f3_30 | 1878 | 6640 |
| hp6e12267_4428467_c2_51 | 1879 | 6641 |
| 12ge20305orf10 | 1880 | 6642 |
| hp2p10272_14094708_f2_9 | 1881 | 6643 |
| hpyhp2p10272orf_0002.aa | 1882 | 6644 |
| hp6p21623_14882837_f3_24 | 1883 | 6645 |
| hp2p10272orf4 | 1884 | 6646 |
| hpyhp2p10272orf_0001.aa | 1885 | 6647 |
| 02ap11117_14882837_c2_67 | 1886 | 6648 |
| hp5e12982_415907_f1_2 | 1887 | 6649 |
| hp5e12982orf5 | 1888 | 6650 |
| 14480927 | 1889 | 6651 |
| 02ge10116_24417562_c2_26 | 1890 | 6652 |
| 02ge10116_24417562_c2_123 | 1891 | 6653 |
| 02ge20116orf33 | 1892 | 6654 |
| hp5e15276orf8 | 1893 | 6655 |
| 02ge10116_4765638_c3_145 | 1894 | 6656 |
| 02ge10116_9956415_c1_83 | 1895 | 6657 |
| 04ap10921orf3 | 1896 | 6658 |
| hpy04ap10921orf_0001.aa | 1897 | 6659 |
| 02ge11810_26359537_f2_5 | 1898 | 6660 |
| 13ee10216orf42 | 1899 | 6661 |
| 13ee10216orf8 | 1900 | 6662 |
| 02ge11622_4897842_f2_19 | 1901 | 6663 |
| 09ge21419orf1 | 1902 | 6664 |
| 01ee60820_14183378_f2_18 | 1903 | 6665 |
| 03ce11207_14183378_f3_150 | 1904 | 6666 |
| 11gp10904orf14 | 1905 | 6667 |
| 11gp10904orf13 | 1906 | 6668 |
| 11gp20904_4414717_f2_13 | 1907 | 6669 |
| 01ee60820_4414717_f1_6 | 1908 | 6670 |
| 01ce10320orf6 | 1909 | 6671 |
| hpy01ce10320orf_0001.aa | 1910 | 6672 |
| 01ce10320_26054633_f3_8 | 1911 | 6673 |
| 01ce10320orf4 | 1912 | 6674 |
| 05ep10815orf2 | 1913 | 6675 |
| hpy05ep10815orf_0001.aa | 1914 | 6676 |
| 05ep10815_7818_c1_89 | 1915 | 6677 |
| 03ce11207_7818_c1_187 | 1916 | 6678 |
| hpy05ae20220orf_0042.aa | 1917 | 6679 |
| 05ae20220orf94 | 1918 | 6680 |
| 05ae30220_24507712_c2_80 | 1919 | 6681 |
| 02ce10213orf10 | 1920 | 6682 |
| 02ce10213orf3 | 1921 | 6683 |
| hp5p15212_23635441_f3_17 | 1922 | 6684 |
| 02ce10213orf18 | 1923 | 6685 |
| hp5p15212_4687538_f2_7 | 1924 | 6686 |
| 06ep11202_133293_c1_19 | 1925 | 6687 |
| 06cp20302orf3 | 1926 | 6688 |
| 04cp11202_36522218_c3_113 | 1927 | 6689 |
| 04ge11713orf40 | 1928 | 6690 |
| 04cp11202_5114015_f3_38 | 1929 | 6691 |
| 04ge11713orf20 | 1930 | 6692 |
| hpyhp2e10911orf_0009.aa | 1931 | 6693 |
| hp2e10911_34173401_c2_90 | 1932 | 6694 |
| hpyhp2e10911orf_0008.aa | 1933 | 6695 |
| 07ap11213_35156577_c1_24 | 1934 | 6696 |
| hp2e10911orf35 | 1935 | 6697 |
| 07ee50709_35156577_f3_80 | 1936 | 6698 |
| 09ze10333_4710386_f2_8 | 1937 | 6699 |
| 03ge10505orf3 | 1938 | 6700 |
| 09ze10333_4710386_f2_7 | 1939 | 6701 |
| 1408 | 1940 | 6702 |
| 09cp10713_30470301_c2_31 | 1941 | 6703 |
| 09cp10713_30470301_c2_30 | 1942 | 6704 |
| 09cp10713orf29 | 1943 | 6705 |
| 12ge10321_6923512_f2_8 | 1944 | 6706 |
| hp4p11352orf7 | 1945 | 6707 |
| 06ep10306orf4 | 1946 | 6708 |
| 03ae10804_976702_c2_34 | 1947 | 6709 |
| 06ep10306orf13 | 1948 | 6710 |
| hp2e10619orf2 | 1949 | 6711 |
| hp2e10619orf1 | 1950 | 6712 |
| hp4e13394_16984790_f2_41 | 1951 | 6713 |
| hpy11cp10916orf_0002.aa | 1952 | 6714 |
| hp4e53394_4556458_f1_12 | 1953 | 6715 |
| hp4e53394_16211062_f3_61 | 1954 | 6716 |
| 03ee11215_23985910_c3_33 | 1955 | 6717 |
| hp4e53394_23985910_c3_117 | 1956 | 6718 |
| 03ee11215orf34 | 1957 | 6719 |
| 03ee11215_35960937_f1_6 | 1958 | 6720 |
| hp4e53394_35960937_f1_9 | 1959 | 6721 |
| 03ee11215orf9 | 1960 | 6722 |
| 01ge10203_16689377_c1_11 | 1961 | 6723 |
| 01ge10203orf8 | 1962 | 6724 |
| 05ae11506orf3 | 1963 | 6725 |
| 01ge10203orf11 | 1964 | 6726 |
| hp1p13939_22656457_c2_34 | 1965 | 6727 |
| 01ge10203_22656457_c2_15 | 1966 | 6728 |
| hp4p62853_22656457_c1_35 | 1967 | 6729 |
| 06ep30223_23557202_c2_130 | 1968 | 6730 |
| hp3e11024orf49 | 1969 | 6731 |
| hpy02ce11220orf_0001.aa | 1970 | 6732 |
| 05ee10411orf5 | 1971 | 6733 |
| hpy05ee10411orf_0001.aa | 1972 | 6734 |
| 06ep30223_5109443_c1_109 | 1973 | 6735 |
| 10407625 | 1974 | 6736 |
| 02ae11612_24335942_c2_84 | 1975 | 6737 |
| 02ae11612orf26 | 1976 | 6738 |
| 2040717 | 1977 | 6739 |
| 02ae11612_32710417_c1_62 | 1978 | 6740 |
| 02ae11612orf36 | 1979 | 6741 |
| hp5e25328_4490967_c2_126 | 1980 | 6742 |
| hp7e10434_25394812_f2_8 | 1981 | 6743 |
| 06ce10515orf2 | 1982 | 6744 |
| 02ce41018_4976537_f1_7 | 1983 | 6745 |
| hp3p10349orf26 | 1984 | 6746 |
| 02ce41018_35945968_f3_25 | 1985 | 6747 |
| hp3p10349orf22 | 1986 | 6748 |
| hp7p90421_4976537_f3_27 | 1987 | 6749 |
| 11ap20714_20494837_f3_42 | 1988 | 6750 |
| hpy04gp11803orf_0002.aa | 1989 | 6751 |
| 11ap20714_21759681_c1_63 | 1990 | 6752 |
| hp5e15440orf25 | 1991 | 6753 |
| 23442642 | 1992 | 6754 |
| 06ap11418_23442642_f1_6 | 1993 | 6755 |
| hp5e15440orf21 | 1994 | 6756 |
| 11ap20714_2588387_f3_44 | 1995 | 6757 |
| 07cp10301orf2 | 1996 | 6758 |
| 05ap21216orf16 | 1997 | 6759 |
| 05ap21216orf24 | 1998 | 6760 |
| 07ap20216_31912575_c2_24 | 1999 | 6761 |
| 11ap20714_31912575_c2_82 | 2000 | 6762 |
| hp3e10057orf1 | 2001 | 6763 |
| 11ap20714_3228437_f2_28 | 2002 | 6764 |
| 13ep12003orf3 | 2003 | 6765 |
| 13ep12003orf12 | 2004 | 6766 |
| hp6p10606_23437942_f1_5 | 2005 | 6767 |
| 485375 | 9597 | 9709 |
| B.4 Proteins involved in carbohydrate | | |
| metabolism | | |
| 2855006 | 2006 | 6768 |
| 02ge11622_13007837_f1_9 | 2007 | 6769 |
| 13ee10216orf9 | 2008 | 6770 |
| 14257751 | 2009 | 6771 |
| 02ge11622_14257751_f3_37 | 2010 | 6772 |
| 13ee10216orf43 | 2011 | 6773 |
| 02ge11622_23992687_f2_27 | 2012 | 6774 |
| 02ge41622_23992687_f1_16 | 2013 | 6775 |
| 13ee10216orf30 | 2014 | 6776 |
| 02ge11622_3988912_f3_39 | 2015 | 6777 |
| 13ee10216orf45 | 2016 | 6778 |
| hpy13ee10216orf_0027.aa | 2017 | 6779 |
| 02ge11622_683262_f2_25 | 2018 | 6780 |
| 02ge41622_683262_f1_14 | 2019 | 6781 |
| 13ee10216orf28 | 2020 | 6782 |
| 02ge41622_683262_f3_44 | 2021 | 6783 |
| 04ep41903_12110918_f1_7 | 2022 | 6784 |
| 14ce11519orf5 | 2023 | 6785 |
| 14ce11519orf2 | 2024 | 6786 |
| 09ae11601orf6 | 2025 | 6787 |
| 04ep41903_4397717_f3_20 | 2026 | 6788 |
| 14ce11519orf3 | 2027 | 6789 |
| 04ep41903_5084843_f3_19 | 2028 | 6790 |
| 04ep41903_5084843_f2_19 | 2029 | 6791 |
| 09cp10502orf15 | 2030 | 6792 |
| 07ae11008_24400293_c1_40 | 2031 | 6793 |
| 07ce11409orf1 | 2032 | 6794 |
| hpy07ce11409orf_0001.aa | 2033 | 6795 |
| hp3p10168orf1 | 2034 | 6796 |
| hpyhp3p10168orf_0001.aa | 2035 | 6797 |
| 14ee41924_19790902_f3_54 | 2036 | 6798 |
| 14ee41924_24226562_f2_37 | 2037 | 6799 |
| 07ce11409orf6 | 2038 | 6800 |
| 06cp11217_34427203_c2_16 | 2039 | 6801 |
| hp1p13852orf14 | 2040 | 6802 |
| 01ep30520orf7 | 2041 | 6803 |
| 03ge11517orf2 | 2042 | 6804 |
| hpy03ge11517orf_0001.aa | 2043 | 6805 |
| 09ce52017_5080442_f3_18 | 2044 | 6806 |
| 09cp10224_22554651_c2_56 | 2045 | 6807 |
| 09ze10333_5861452_c1_17 | 2046 | 6808 |
| hpy03ge10505orf_0005.aa | 2047 | 6809 |
| 15807794 | 2048 | 6810 |
| 06cp30603_23610930_c3_72 | 2049 | 6811 |
| 05cp20518orf64 | 2050 | 6812 |
| 06cp30603_33443_f3_32 | 2051 | 6813 |
| 05cp20518orf27 | 2052 | 6814 |
| 13ap11119orf1 | 2053 | 6815 |
| hpy13ap11119orf_0001.aa | 2054 | 6816 |
| 05ap12011orf1 | 2055 | 6817 |
| hpy05ap12011orf_0001.aa | 2056 | 6818 |
| 07ce11019_23439204_c1_10 | 2057 | 6819 |
| 14gp11423_25416691_c2_75 | 2058 | 6820 |
| hpy09cp10614orf_0001.aa | 2059 | 6821 |
| 09cp10614orf3 | 2060 | 6822 |
| 12ae10622_4492342_f1_16 | 2061 | 6823 |
| 14gp11423_4492342_c2_74 | 2062 | 6824 |
| 23912807 | 2063 | 6825 |
| hp4e13394_24298127_c2_106 | 2064 | 6826 |
| 09ap11406orf8 | 2065 | 6827 |
| hpy05cp11911orf_0001.aa | 2066 | 6828 |
| 02ee10810orf1 | 2067 | 6829 |
| hpy02ee10810orf_0001.aa | 2068 | 6830 |
| 02ee10810_20179790_f1_1 | 2069 | 6831 |
| 02ae11612_4328518_f3_42 | 2070 | 6832 |
| 01ce21104_4328518_f1_6 | 2071 | 6833 |
| hp7e10429_34250036_c1_25 | 2072 | 6834 |
| hpy12ap11614orf_0006.aa | 2073 | 6835 |
| 12ap11614orf3 | 2074 | 6836 |
| 12ap11614orf2 | 2075 | 6837 |
| 12ap11614_4562712_f2_2 | 2076 | 6838 |
| 13723593 | 9598 | 9710 |
| 24298127 | 9599 | 9711 |
| B.5 Proteins involved in nucleic acid | | |
| metabolism | | |
| 09ae10323orf3 | 2077 | 6839 |
| 09cp61003_4297967_f3_56 | 2078 | 6840 |
| 09cp61003_6142965_f2_37 | 2079 | 6841 |
| 09ae10323orf1 | 2080 | 6842 |
| hpy13ee10216orf_0008.aa | 2081 | 6843 |
| 02ge41622_14469018_f2_25 | 2082 | 6844 |
| 02ge11622_14726062_f1_4 | 2083 | 6845 |
| 13ee10216orf17 | 2084 | 6846 |
| hp6p31058_35156592_f3_1 | 2085 | 6847 |
| 02ge41622_14469018_f3_33 | 2086 | 6848 |
| 3261306 | 2087 | 6849 |
| 06ee10709_7118818_f3_9 | 2088 | 6850 |
| 06ee10709orf5 | 2089 | 6851 |
| 01ae11010_29297936_c3_43 | 2090 | 6852 |
| hp3e11075orf4 | 2091 | 6853 |
| 01ae11010_35581562_c1_37 | 2092 | 6854 |
| 01ae11010_40963_c1_36 | 2093 | 6855 |
| 29ee12019orf1 | 2094 | 6856 |
| 03ce11207_40963_f1_45 | 2095 | 6857 |
| 04gp11701_4687842_c2_29 | 2096 | 6858 |
| hp3e11168orf26 | 2097 | 6859 |
| 06ap10609orf3 | 2098 | 6860 |
| 06ap10609orf2 | 2099 | 6861 |
| hpy06ap10609orf_0001.aa | 2100 | 6862 |
| 06ap10609_3390656_c1_41 | 2101 | 6863 |
| 06cp10411orf1 | 2102 | 6864 |
| 06cp10411orf2 | 2103 | 6865 |
| 06gp71906_16494193_c3_191 | 2104 | 6866 |
| 14574201 | 2105 | 6867 |
| hpy02cp11822orf_0007.aa | 2106 | 6868 |
| 02cp11822orf26 | 2107 | 6869 |
| 04cp11202_4339813_c1_73 | 2108 | 6870 |
| 04cp11202_4339813_c3_107 | 2109 | 6871 |
| 14ee41924_14313885_c3_100 | 2110 | 6872 |
| 14313885 | 2111 | 6873 |
| 11ce10917orf14 | 2112 | 6874 |
| 07ce11409orf11 | 2113 | 6875 |
| hpy07ce11409orf_0005.aa | 2114 | 6876 |
| 07ce11409orf12 | 2115 | 6877 |
| 14ee41924_5080087_c1_57 | 2116 | 6878 |
| 01ep11504orf1 | 2117 | 6879 |
| 01ge10801orf4 | 2118 | 6880 |
| 01ep11504orf4 | 2119 | 6881 |
| 01ep11504orf3 | 2120 | 6882 |
| hpy01ep11504orf_0001.aa | 2121 | 6883 |
| 02ae31010_16828135_f3_32 | 2122 | 6884 |
| hp5p15641_24412842_f1_2 | 2123 | 6885 |
| hp5p15641orf11 | 2124 | 6886 |
| 02ae31010_33203166_f1_13 | 2125 | 6887 |
| hp1p13947orf9 | 2126 | 6888 |
| hpyhp1p13947orf_0007.aa | 2127 | 6889 |
| hp1p13947orf3 | 2128 | 6890 |
| 02ae31010_5080463_f2_26 | 2129 | 6891 |
| 14ae20424orf1 | 2130 | 6892 |
| 04ae61517_5869133_f2_4 | 2131 | 6893 |
| hp4p12313orf4 | 2132 | 6894 |
| hp4p12313orf3 | 2133 | 6895 |
| 09cp10224_22554651_c2_51 | 2134 | 6896 |
| 32144532 | 2135 | 6897 |
| 06cp30603_10739532_f1_5 | 2136 | 6898 |
| 05cp20518orf5 | 2137 | 6899 |
| 04ge10816orf1 | 2138 | 6900 |
| 02ee10520orf7 | 2139 | 6901 |
| 02ee10520orf4 | 2140 | 6902 |
| hpy02ee10520orf_0005.aa | 2141 | 6903 |
| 04ge10816orf3 | 2142 | 6904 |
| hpy04ge10816orf_0001.aa | 2143 | 6905 |
| 13ae10610_3962568_c3_37 | 2144 | 6906 |
| 04ge10816orf2 | 2145 | 6907 |
| 13ae10610_16407276_c2_31 | 2146 | 6908 |
| 13ae10610_3962568_c1_26 | 2147 | 6909 |
| 12ge10321_4298468_f3_15 | 2148 | 6910 |
| hp4p11352orf10 | 2149 | 6911 |
| 09cp10405orf3 | 2150 | 6912 |
| 09cp10405orf1 | 2151 | 6913 |
| hpy09cp10405orf_0001.aa | 2152 | 6914 |
| hp4e13394_4119017_c2_95 | 2153 | 6915 |
| hpy01ep30520orf_0002.aa | 2154 | 6916 |
| 01ep30520orf8 | 2155 | 6917 |
| hpy01ep30520orf_0001.aa | 2156 | 6918 |
| 06ep30223_23476067_c1_119 | 2157 | 6919 |
| 06ep30223_23476067_c1_115 | 2158 | 6920 |
| 06ep30223_23476067_c1_118 | 2159 | 6921 |
| 6517040 | 2160 | 6922 |
| 06ep30223_33600817_f2_63 | 2161 | 6923 |
| hp3e11024orf24 | 2162 | 6924 |
| 867183 | 2163 | 6925 |
| 06ep11917_21522805_c3_25 | 2164 | 6926 |
| hp4e14535orf4 | 2165 | 6927 |
| hp6p12129_16192812_c2_22 | 2166 | 6928 |
| 01ep12002_16192812_c1_13 | 2167 | 6929 |
| 04ep10515orf8 | 2168 | 6930 |
| hp2e10692orf3 | 2169 | 6931 |
| 14ce61516_6025312_f1_6 | 2170 | 6932 |
| hpy05ae10808orf_0003.aa | 2171 | 6933 |
| 11ae80818_6900292_c3_67 | 2172 | 6934 |
| 11ae80818_976506_f2_20 | 2173 | 6935 |
| hpy06cp11722orf_0004.aa | 2174 | 6936 |
| 04ae21413_26360918_f3_2 | 2175 | 6937 |
| 04ae11413orf3 | 2176 | 6938 |
| 04ae11413orf1 | 2177 | 6939 |
| 04ae21413_19532530_f1_1 | 2178 | 6940 |
| 11ap20714_34663910_f3_29 | 2179 | 6941 |
| 04ae11413orf2 | 2180 | 6942 |
| hp5e15440orf7 | 2181 | 6943 |
| hp8e10065_4962812_f2_18 | 2182 | 6944 |
| 1581937 | 9600 | 9712 |
| B.6 Proteins involved in lipid metabolism | | |
| hp6e12267_14650278_f3_29 | 2183 | 6945 |
| 12ge20305orf26 | 2184 | 6946 |
| 13ee10216orf1 | 2185 | 6947 |
| hpy13ee10216orf_0001.aa | 2186 | 6948 |
| 02ge11622_26023917_f1_1 | 2187 | 6949 |
| 02ge41622_33985402_f1_1 | 2188 | 6950 |
| hp3e11060orf16 | 2189 | 6951 |
| 09ae10512_4334717_f1_7 | 2190 | 6952 |
| 422937 | 2191 | 6953 |
| 29zp10241orf11 | 2192 | 6954 |
| 14cp20718_22767327_c3_19 | 2193 | 6955 |
| hp5p15861_16579692_f2_7 | 2194 | 6956 |
| 02cp20821orf2 | 2195 | 6957 |
| 14ee41924_23834800_f2_32 | 2196 | 6958 |
| 11ce10917orf10 | 2197 | 6959 |
| 14ee41924_6672317_f2_30 | 2198 | 6960 |
| 11ce10917orf8 | 2199 | 6961 |
| 32705252 | 2200 | 6962 |
| hp4p11352orf6 | 2201 | 6963 |
| hp4p11352orf2 | 2202 | 6964 |
| 12ge10321_35445843_f2_7 | 2203 | 6965 |
| 06ep11917_19539202_c1_21 | 2204 | 6966 |
| 43490713 | 2205 | 6967 |
| 06ep11917_5114568_c2_23 | 2206 | 6968 |
| hp4e14535orf2 | 2207 | 6969 |
| 14ce61516_16814158_f1_5 | 2208 | 6970 |
| 14ce61516_14162751_f3_14 | 2209 | 6971 |
| hp2e10692orf2 | 2210 | 6972 |
| hp2e10692orf1 | 2211 | 6973 |
| 02ce21018orf4 | 2212 | 6974 |
| 02ce41018_24314075_c3_46 | 2213 | 6975 |
| 11ap20714_4578452_f1_8 | 2214 | 6976 |
| 07ap20216_4578452_f1_5 | 2215 | 6977 |
| 05ap21216orf8 | 2216 | 6978 |
| hp1p14013_35179683_f2_6 | 2217 | 6979 |
| hp1p14013orf3 | 2218 | 6980 |
| 5138 | 9601 | 9713 |
| 35445843 | 9602 | 9714 |
| B.7 Proteins involved in mRNA translation | | |
| and ribosome biogenesis | | |
| 05ee20322orf13 | 2219 | 6981 |
| 02ge10116_36464702_f3_70 | 2220 | 6982 |
| 03ap10105orf2 | 2221 | 6983 |
| 03ap10105orf1 | 2222 | 6984 |
| hpy11gp10904orf_0001.aa | 2223 | 6985 |
| 01ee60820_36132778_c3_43 | 2224 | 6986 |
| 03ce11207_36132778_c2_225 | 2225 | 6987 |
| 785437 | 2226 | 6988 |
| 01ae11010_5192143_c3_32 | 2227 | 6989 |
| hp4e14522orf11 | 2228 | 6990 |
| 05ep10815_5318766_c1_87 | 2229 | 6991 |
| 07ge11504orf7 | 2230 | 6992 |
| 11gp10904orf24 | 2231 | 6993 |
| 11gp20904_6136000_c2_29 | 2232 | 6994 |
| 11gp20904_791318_c3_32 | 2233 | 6995 |
| 11gp10904orf28 | 2234 | 6996 |
| 05ae30220_14647183_c2_83 | 2235 | 6997 |
| 05ae20220orf72 | 2236 | 6998 |
| 05gp11901orf27 | 2237 | 6999 |
| 05gp11901orf21 | 2238 | 7000 |
| hp5p15496orf1 | 2239 | 7001 |
| 11ee11408_3955010_c1_36 | 2240 | 7002 |
| 04ee11108_4801318_f1_8 | 2241 | 7003 |
| 27ze10351orf6 | 2242 | 7004 |
| 06ae11122orf1 | 2243 | 7005 |
| hpy06ae11122orf_0001.aa | 2244 | 7006 |
| 05ae30801_22556543_c2_19 | 2245 | 7007 |
| 05ae30801_24792212_c1_16 | 2246 | 7008 |
| hpyhp1e10446orf_0002.aa | 2247 | 7009 |
| 05ae30801_26306562_c3_23 | 2248 | 7010 |
| 05ae30801_33204692_c2_21 | 2249 | 7011 |
| 05ae30801_33290942_c2_17 | 2250 | 7012 |
| hp1e10446orf5 | 2251 | 7013 |
| 05ae30801_34196062_c3_24 | 2252 | 7014 |
| 05ae30801_4955468_c2_18 | 2253 | 7015 |
| 14gp11419orf1 | 2254 | 7016 |
| 24500088 | 2255 | 7017 |
| hp5p15212_806540_f2_8 | 2256 | 7018 |
| 02ce10213orf2 | 2257 | 7019 |
| 01ae12001_3907688_c2_37 | 2258 | 7020 |
| 07gp11807orf45 | 2259 | 7021 |
| hp6e10967_4506518_c1_16 | 2260 | 7022 |
| hp2p10625orf24 | 2261 | 7023 |
| 06ep11202_3956713_c2_25 | 2262 | 7024 |
| 14ge10402orf1 | 2263 | 7025 |
| 02cp11822orf23 | 2264 | 7026 |
| 02cp11822orf18 | 2265 | 7027 |
| 04cp11202_4490968_c2_78 | 2266 | 7028 |
| 04cp11202_4490968_c3_113 | 2267 | 7029 |
| hpy02cp11822orf_0004.aa | 2268 | 7030 |
| 4895327 | 2269 | 7031 |
| 04cp11202_4895327_c1_74 | 2270 | 7032 |
| 04cp11202_4895327_c3_108 | 2271 | 7033 |
| 02cp11822orf22 | 2272 | 7034 |
| 06gp71906_4895327_f3_94 | 2273 | 7035 |
| 07ae11008_24023588_c2_42 | 2274 | 7036 |
| hp5e15003orf1 | 2275 | 7037 |
| hp5p15003orf2 | 2276 | 7038 |
| hpyhp5p15003orf_0001.aa | 2277 | 7039 |
| 14gp11820orf5 | 2278 | 7040 |
| hpy14gp11820orf_0001.aa | 2279 | 7041 |
| 07ae11008_26258557_c3_52 | 2280 | 7042 |
| 09cp20502_26258557_c2_41 | 2281 | 7043 |
| 14gp11820orf2 | 2282 | 7044 |
| 14gp11820orf11 | 2283 | 7045 |
| 09cp20502_4562512_c1_32 | 2284 | 7046 |
| hp5e15003orf2 | 2285 | 7047 |
| 07ae11008_6680318_c2_41 | 2286 | 7048 |
| 14cp20718_24250207_c3_18 | 2287 | 7049 |
| 29zp10241orf13 | 2288 | 7050 |
| hpy07ap11111orf_0003.aa | 2289 | 7051 |
| 07ap11111orf20 | 2290 | 7052 |
| hpy07ap11111orf_0001.aa | 2291 | 7053 |
| 07ap11111_26172193_c1_13 | 2292 | 7054 |
| 14cp12009orf5 | 2293 | 7055 |
| 14cp12009orf2 | 2294 | 7056 |
| hpy14cp12009orf_0001.aa | 2295 | 7057 |
| 01ap10322orf2 | 2296 | 7058 |
| 07ap11111_31884687_f2_7 | 2297 | 7059 |
| 07ap11111orf5 | 2298 | 7060 |
| hpy14cp12009orf_0003.aa | 2299 | 7061 |
| 14cp12009orf4 | 2300 | 7062 |
| 07ap61111_31884687_f3_39 | 2301 | 7063 |
| 07ap61111_31884687_f3_41 | 2302 | 7064 |
| 14ee41924_16282067_c1_72 | 2303 | 7065 |
| 02ep30607orf32 | 2304 | 7066 |
| 02ep30607orf23 | 2305 | 7067 |
| 14ee41924_19565702_c1_69 | 2306 | 7068 |
| 07ee11402_19565702_c2_88 | 2307 | 7069 |
| hp5p15861_26360952_f3_11 | 2308 | 7070 |
| 02cp20821orf7 | 2309 | 7071 |
| 01ce11618orf50 | 2310 | 7072 |
| 01ce11618orf32 | 2311 | 7073 |
| 29ge30321_212832_c2_22 | 2312 | 7074 |
| 07ap11213_22400307_c2_32 | 2313 | 7075 |
| hp2e10911orf29 | 2314 | 7076 |
| hp5p15641_34101462_f2_8 | 2315 | 7077 |
| hp5p15641orf3 | 2316 | 7078 |
| 07ap11213_4897193_c1_25 | 2317 | 7079 |
| hp2e10911orf48 | 2318 | 7080 |
| 02cp11404orf4 | 2319 | 7081 |
| hpy02cp11404orf_0001.aa | 2320 | 7082 |
| 02cp11404orf8 | 2321 | 7083 |
| 07ge11521_2579708_c2_30 | 2322 | 7084 |
| 02cp11404orf7 | 2323 | 7085 |
| 07ge11521_16992087_c1_26 | 2324 | 7086 |
| 07ge11521_16992087_c2_33 | 2325 | 7087 |
| 33601578 | 2326 | 7088 |
| 07ge11521_33601578_c3_36 | 2327 | 7089 |
| 02cp11404orf11 | 2328 | 7090 |
| 07ge11521_4476568_c3_37 | 2329 | 7091 |
| 02cp11404orf12 | 2330 | 7092 |
| 07ge11521_5859713_c2_31 | 2331 | 7093 |
| 02cp11404orf2 | 2332 | 7094 |
| hp1p11244orf8 | 2333 | 7095 |
| 01ee20804orf4 | 2334 | 7096 |
| hpy01ee20804orf_0002.aa | 2335 | 7097 |
| 09cp10224_3906717_c2_50 | 2336 | 7098 |
| 09cp10224_4720437_c2_47 | 2337 | 7099 |
| hp1p11244orf1 | 2338 | 7100 |
| 11ce11005orf1 | 2339 | 7101 |
| 06cp30603_16523452_c2_95 | 2340 | 7102 |
| 05cp20518orf16 | 2341 | 7103 |
| 05cp20518orf26 | 2342 | 7104 |
| 06cp30603_24886308_f2_15 | 2343 | 7105 |
| 06cp30603_24886308_f2_27 | 2344 | 7106 |
| 05cp20518orf17 | 2345 | 7107 |
| 05cp20518orf2 | 2346 | 7108 |
| 06cp30603_34178762_f1_3 | 2347 | 7109 |
| 09cp10713_5194218_c1_25 | 2348 | 7110 |
| 09cp10713orf21 | 2349 | 7111 |
| 09cp10713orf28 | 2350 | 7112 |
| 09cp10713_23852057_c2_30 | 2351 | 7113 |
| 09cp10713_5194218_c2_29 | 2352 | 7114 |
| hpy07ap11112orf_0001.aa | 2353 | 7115 |
| 07ap11112orf1 | 2354 | 7116 |
| hp3e10946_34175837_f3_3 | 2355 | 7117 |
| hp3e10946_32609412_f3_4 | 2356 | 7118 |
| hp3p10048orf3 | 2357 | 7119 |
| hp4e13394_1209687_c2_94 | 2358 | 7120 |
| 01ep10216orf5 | 2359 | 7121 |
| hpy01ep10216orf_0003.aa | 2360 | 7122 |
| hp4e13394_14554676_f1_19 | 2361 | 7123 |
| hp4e13394_3942318_f1_25 | 2362 | 7124 |
| 06ep11108orf24 | 2363 | 7125 |
| hp4e13394_415657_f1_23 | 2364 | 7126 |
| 06ep11108orf18 | 2365 | 7127 |
| 07ap11622orf14 | 2366 | 7128 |
| 06ae11020orf1 | 2367 | 7129 |
| 07ap11622orf10 | 2368 | 7130 |
| hp1e13852_23599087_f1_1 | 2369 | 7131 |
| 07gp10713orf3 | 2370 | 7132 |
| 06ep30223_34244037_c2_139 | 2371 | 7133 |
| 06ep30223_6110937_c2_140 | 2372 | 7134 |
| 01ee10520orf1 | 2373 | 7135 |
| hpy01ee10520orf_0001.aa | 2374 | 7136 |
| 06ep30223_4035262_c1_117 | 2375 | 7137 |
| hp5e15555_4035262_c3_166 | 2376 | 7138 |
| 02ae11612_23598811_c1_63 | 2377 | 7139 |
| 02ae11612orf37 | 2378 | 7140 |
| 02ae11612_26835305_f3_43 | 2379 | 7141 |
| 05cp11911orf7 | 2380 | 7142 |
| 02ae11612_4488967_f2_29 | 2381 | 7143 |
| 05cp11911orf16 | 2382 | 7144 |
| hp3e11086orf9 | 2383 | 7145 |
| hp3e11086orf4 | 2384 | 7146 |
| hp3e11086orf5 | 2385 | 7147 |
| hp5p15575_15900317_f3_23 | 2386 | 7148 |
| hp5p15575_23438812_f2_15 | 2387 | 7149 |
| 14ee11113orf1 | 2388 | 7150 |
| hp5p15575_33478126_f2_14 | 2389 | 7151 |
| hp5p15575_4886087_f3_25 | 2390 | 7152 |
| hp6e10491_25595443_c1_14 | 2391 | 7153 |
| hpyhp5e15084orf_0003.aa | 2392 | 7154 |
| hp6e10491_34415816_c1_16 | 2393 | 7155 |
| 03ee10521orf2 | 2394 | 7156 |
| hp6e10491_36385438_c3_21 | 2395 | 7157 |
| hp5e15084orf14 | 2396 | 7158 |
| 04ee30405_3911693_f3_6 | 2397 | 7159 |
| hpy16ae10508orf_0010.aa | 2398 | 7160 |
| hpy16ae10508orf_0009.aa | 2399 | 7161 |
| 16ae10508orf18 | 2400 | 7162 |
| 07cp11107orf1 | 2401 | 7163 |
| 07cp11107orf2 | 2402 | 7164 |
| 16ae10508orf19 | 2403 | 7165 |
| hp6p10509_12695436_c2_15 | 2404 | 7166 |
| 07ep11916_10973880_c1_19 | 2405 | 7167 |
| 01cp11414orf1 | 2406 | 7168 |
| hp6p12244_34157500_f1_3 | 2407 | 7169 |
| 02ae11211orf5 | 2408 | 7170 |
| 07ep11916_4042162_c3_30 | 2409 | 7171 |
| 01cp11414orf6 | 2410 | 7172 |
| hp6p12244_4883452_f3_33 | 2411 | 7173 |
| 02ae11211orf1 | 2412 | 7174 |
| 11ae12004_26275300_c3_48 | 2413 | 7175 |
| hpy14cp10705orf_0002.aa | 2414 | 7176 |
| hp7e10433_32228441_f2_4 | 2415 | 7177 |
| hp5p15504orf8 | 2416 | 7178 |
| 14ce21516_22394702_f2_2 | 2417 | 7179 |
| 14ce21516orf2 | 2418 | 7180 |
| 14ce61516_7031692_f1_1 | 2419 | 7181 |
| hp7e10590_33406577_c2_91 | 2420 | 7182 |
| 06cp11722orf20 | 2421 | 7183 |
| 11ae80818_3961000_f2_18 | 2422 | 7184 |
| 25595387 | 2423 | 7185 |
| hp7e10434_25595387_f3_16 | 2424 | 7186 |
| 06ce10515orf4 | 2425 | 7187 |
| 06ae10902_1209842_f2_2 | 2426 | 7188 |
| 06ae10902orf1 | 2427 | 7189 |
| hp7p10325_22861641_f2_2 | 2428 | 7190 |
| 06ae10902_24610937_f3_7 | 2429 | 7191 |
| 06ae10902orf2 | 2430 | 7192 |
| 06ae10902_26758380_f3_6 | 2431 | 7193 |
| 06ap11418_548837_c2_25 | 2432 | 7194 |
| hp5e15440orf9 | 2433 | 7195 |
| 13ep12003orf14 | 2434 | 7196 |
| 4177212 | 2435 | 7197 |
| 01ee11621orf6 | 2436 | 7198 |
| 13ep12003orf11 | 2437 | 7199 |
| hp6p10606_1875_f1_10 | 2438 | 7200 |
| 24803280 | 9603 | 9715 |
| 32036462 | 9604 | 9716 |
| “B.8 Proteins involved in genome | | |
| replication, transcription, recombination and | | |
| repair” | | |
| 11719687 | 2439 | 7201 |
| hp3p10156orf8 | 2440 | 7202 |
| hp7p10290_1994062_f1_1 | 2441 | 7203 |
| hp6e12267_23955008_f1_2 | 2442 | 7204 |
| 12ge20305orf34 | 2443 | 7205 |
| hp2p10272_26354187_c2_36 | 2444 | 7206 |
| 09ap20802orf28 | 2445 | 7207 |
| hp2p10272_4899052_c3_46 | 2446 | 7208 |
| 09ap20802orf34 | 2447 | 7209 |
| hp3e10950orf2 | 2448 | 7210 |
| 02gp20706_32242202_c3_57 | 2449 | 7211 |
| hp6p12311_12894031_c3_12 | 2450 | 7212 |
| 05ep20322orf5 | 2451 | 7213 |
| hp6p12311_4110627_c2_11 | 2452 | 7214 |
| 05ep20322orf3 | 2453 | 7215 |
| 02ge10116_4110627_f3_70 | 2454 | 7216 |
| 06ee10709orf11 | 2455 | 7217 |
| 06ee10709orf18 | 2456 | 7218 |
| 06ee10709_24228168_c1_12 | 2457 | 7219 |
| 01cp20708_12289077_f2_13 | 2458 | 7220 |
| 14ee10419orf7 | 2459 | 7221 |
| 05ep10815_16305252_c3_111 | 2460 | 7222 |
| 16305252 | 2461 | 7223 |
| 07ge11504orf4 | 2462 | 7224 |
| 05ep10815_16600757_f2_41 | 2463 | 7225 |
| 01xe21717orf27 | 2464 | 7226 |
| 01cp20708_203167_c3_61 | 2465 | 7227 |
| 14ee10419orf14 | 2466 | 7228 |
| hp3e11168orf7 | 2467 | 7229 |
| hp3e11168orf6 | 2468 | 7230 |
| 04gp11701_23625010_f3_21 | 2469 | 7231 |
| 14ee10419orf17 | 2470 | 7232 |
| hpy14ee10419orf_0010.aa | 2471 | 7233 |
| hp3e11168orf36 | 2472 | 7234 |
| 04gp11701_39068_c1_25 | 2473 | 7235 |
| 14ee10419orf18 | 2474 | 7236 |
| 01cp20708_39068_c2_52 | 2475 | 7237 |
| 07ap80601orf9 | 2476 | 7238 |
| 07ap80601orf4 | 2477 | 7239 |
| 07ap80601_19726452_f1_3 | 2478 | 7240 |
| 11ee11408_23603162_c1_38 | 2479 | 7241 |
| hp5p15496orf7 | 2480 | 7242 |
| 11ee11408_7927_c1_37 | 2481 | 7243 |
| hp5p15496orf2 | 2482 | 7244 |
| 07ap80601orf3 | 2483 | 7245 |
| 07ap80601_24808433_f1_2 | 2484 | 7246 |
| 07ap80601_976413_f3_9 | 2485 | 7247 |
| 07ap80601orf2 | 2486 | 7248 |
| 05ae30220_976413_c3_204 | 2487 | 7249 |
| 09ae10512_23618812_c3_64 | 2488 | 7250 |
| hp3e11060orf6 | 2489 | 7251 |
| 09ae10512_23860625_c1_41 | 2490 | 7252 |
| hp3e11060orf10 | 2491 | 7253 |
| 09ae10512_24274162_c1_39 | 2492 | 7254 |
| hp3e11060orf8 | 2493 | 7255 |
| 24818802 | 2494 | 7256 |
| 09ae10512_24818802_c2_49 | 2495 | 7257 |
| hp3e11060orf2 | 2496 | 7258 |
| 03ce21717orf2 | 2497 | 7259 |
| hp1p10488orf7 | 2498 | 7260 |
| 06ae11016_25431251_f2_22 | 2499 | 7261 |
| 03ae10516orf13 | 2500 | 7262 |
| 06ap30322_1213427_c1_26 | 2501 | 7263 |
| 01ae12001_19695883_c2_45 | 2502 | 7264 |
| 07gp11807orf55 | 2503 | 7265 |
| 06ap10609_19695883_c1_38 | 2504 | 7266 |
| 24036302 | 2505 | 7267 |
| 01ae12001_23625827_c3_54 | 2506 | 7268 |
| 07gp11807orf35 | 2507 | 7269 |
| 14ap10221_36615625_c2_3 | 2508 | 7270 |
| 11ee11408_36615625_c3_52 | 2509 | 7271 |
| 05gp11901orf26 | 2510 | 7272 |
| 01ae12001_7320277_c3_53 | 2511 | 7273 |
| 07gp11807orf34 | 2512 | 7274 |
| 29ep10720_21677217_f2_9 | 2513 | 7275 |
| 11ge10309orf29 | 2514 | 7276 |
| 06ap11119_975187_f3_22 | 2515 | 7277 |
| hp6p10723orf6 | 2516 | 7278 |
| 06cp11118_117325_f3_12 | 2517 | 7279 |
| 01ce11513orf10 | 2518 | 7280 |
| 01ce11513orf11 | 2519 | 7281 |
| 01ce11513orf2 | 2520 | 7282 |
| 06cp11118_24406312_f2_7 | 2521 | 7283 |
| 05ap11505orf7 | 2522 | 7284 |
| hpy05ap11505orf_0007.aa | 2523 | 7285 |
| 11ge10309orf11 | 2524 | 7286 |
| 14ap11617_2040750_f1_1 | 2525 | 7287 |
| hp6e10967_10834443_c3_26 | 2526 | 7288 |
| hp2p10625orf15 | 2527 | 7289 |
| 05ee10816_34258562_f1_2 | 2528 | 7290 |
| 04ce11617orf11 | 2529 | 7291 |
| hpy05ee10816orf_0001.aa | 2530 | 7292 |
| hpy05ee10816orf_0004.aa | 2531 | 7293 |
| 05ee10816orf2 | 2532 | 7294 |
| 05ee10816orf1 | 2533 | 7295 |
| 06ep10615_34662502_f1_11 | 2534 | 7296 |
| 06ep10615_34662502_f1_11 | 2535 | 7297 |
| 04ce11617orf1 | 2536 | 7298 |
| hpy04ce11617orf_0001.aa | 2537 | 7299 |
| 05ee10816_36199207_f3_14 | 2538 | 7300 |
| 06ep10615_36199207_f1_12 | 2539 | 7301 |
| 06ep10615_34662502_f2_26 | 2540 | 7302 |
| 04ce11617orf26 | 2541 | 7303 |
| 04ce11617orf27 | 2542 | 7304 |
| 05ee10816_4687651_c1_22 | 2543 | 7305 |
| 06cp20302orf9 | 2544 | 7306 |
| 06ep11202_104706_c3_33 | 2545 | 7307 |
| 06ep11202_26179002_c1_20 | 2546 | 7308 |
| 06cp20302orf4 | 2547 | 7309 |
| 06ep11202_26179002_c3_32 | 2548 | 7310 |
| 06ep11202_3020312_f1_1 | 2549 | 7311 |
| 06ep11202orf1 | 2550 | 7312 |
| hpy06ep11202orf_0001.aa | 2551 | 7313 |
| hpy14ce20219orf_0004.aa | 2552 | 7314 |
| 06gp71906_36125062_f1_1 | 2553 | 7315 |
| 07ap61111_23992002_c1_66 | 2554 | 7316 |
| 07cp10312_23992002_f2_5 | 2555 | 7317 |
| hpyhp1p13868orf_0007.aa | 2556 | 7318 |
| 07ap61111_24406593_c3_108 | 2557 | 7319 |
| 11cp10113orf5 | 2558 | 7320 |
| hpy11cp10113orf_0003.aa | 2559 | 7321 |
| hpy01ap10322orf_0001.aa | 2560 | 7322 |
| 07ap61111_25599087_f2_20 | 2561 | 7323 |
| 14ap10815_5274093_f2_5 | 2562 | 7324 |
| hp3e10349orf9 | 2563 | 7325 |
| 260941 | 2564 | 7326 |
| 04gp11213orf36 | 2565 | 7327 |
| 14ap10815_6312_c2_22 | 2566 | 7328 |
| hp3e10349orf18 | 2567 | 7329 |
| 07cp10312_24415633_f1_3 | 2568 | 7330 |
| hpyhp1p13868orf_0006.aa | 2569 | 7331 |
| 14ee41924_23598962_f2_23 | 2570 | 7332 |
| 23598962 | 2571 | 7333 |
| 02ep30607orf10 | 2572 | 7334 |
| 4882652 | 2573 | 7335 |
| hp5p15861_4015682_f1_1 | 2574 | 7336 |
| 02cp20821orf10 | 2575 | 7337 |
| 14ee41924_4165950_f2_25 | 2576 | 7338 |
| 02ep30607orf12 | 2577 | 7339 |
| 07ee30709_16681432_f2_2 | 2578 | 7340 |
| 07ae11020orf4 | 2579 | 7341 |
| 07ee50709_32613307_f1_2 | 2580 | 7342 |
| 07ae11020orf2 | 2581 | 7343 |
| 07ee30709_1000_f3_3 | 2582 | 7344 |
| 07ee50709_36031953_f3_76 | 2583 | 7345 |
| 32627125 | 2584 | 7346 |
| 09ce52017_35283337_c2_25 | 2585 | 7347 |
| hpy01ep30520orf_0006.aa | 2586 | 7348 |
| hpy01ep30520orf_0007.aa | 2587 | 7349 |
| 01ep30520orf20 | 2588 | 7350 |
| 09ce52017_32627125_c3_30 | 2589 | 7351 |
| 09ce52017_32627125_c2_25 | 2590 | 7352 |
| 11ge10309orf51 | 2591 | 7353 |
| 29ep10720_24495437_f3_22 | 2592 | 7354 |
| 29ep10720_34553192_f1_7 | 2593 | 7355 |
| 23440814 | 2594 | 7356 |
| hpy11ge10309orf_0031.aa | 2595 | 7357 |
| 29ep10720_4099093_c1_23 | 2596 | 7358 |
| 02ap21102_4099093_c2_30 | 2597 | 7359 |
| 05ap11505orf1 | 2598 | 7360 |
| 06cp30603_12375083_c2_57 | 2599 | 7361 |
| 05cp20518orf51 | 2600 | 7362 |
| 12ae10622_13863791_f3_16 | 2601 | 7363 |
| 05cp20518orf56 | 2602 | 7364 |
| 04gp11213orf23 | 2603 | 7365 |
| 04gp11213orf6 | 2604 | 7366 |
| hp6e20339_23556318_c2_47 | 2605 | 7367 |
| hp6e20339_24391525_c2_45 | 2606 | 7368 |
| 06cp30603_24391525_f3_70 | 2607 | 7369 |
| 04gp11213orf21 | 2608 | 7370 |
| 06cp30603_32663437_c1_48 | 2609 | 7371 |
| 11ge61517_24429687_c2_161 | 2610 | 7372 |
| 05cp20518orf65 | 2611 | 7373 |
| hp6e20339_2523462_c3_56 | 2612 | 7374 |
| 04gp11213orf4 | 2613 | 7375 |
| hp3e10302orf13 | 2614 | 7376 |
| 06cp30603_33250211_f3_65 | 2615 | 7377 |
| 11cp10410_33250211_f2_2 | 2616 | 7378 |
| hpy04gp11213orf_0023.aa | 2617 | 7379 |
| 04gp11213orf27 | 2618 | 7380 |
| 03gp11402orf1 | 2619 | 7381 |
| hpy03gp11402orf_0001.aa | 2620 | 7382 |
| hp6e20339_963963_f2_15 | 2621 | 7383 |
| 06cp30603_33989092_c1_78 | 2622 | 7384 |
| 23880087 | 2623 | 7385 |
| 06cp30603_34174212_c3_71 | 2624 | 7386 |
| 05cp20518orf63 | 2625 | 7387 |
| 06cp30603_3942318_c3_155 | 2626 | 7388 |
| 05cp20518orf55 | 2627 | 7389 |
| 06cp30603_3942318_c2_61 | 2628 | 7390 |
| 06cp30603_4881577_f2_17 | 2629 | 7391 |
| 06cp30603_4881577_f1_5 | 2630 | 7392 |
| 05cp20518orf19 | 2631 | 7393 |
| 04gp11213orf16 | 2632 | 7394 |
| 04gp11213orf26 | 2633 | 7395 |
| hp6e20339_10197827_c3_59 | 2634 | 7396 |
| hp6e20339_5272277_c2_49 | 2635 | 7397 |
| 04gp11213orf8 | 2636 | 7398 |
| 06cp30603_5272277_f1_22 | 2637 | 7399 |
| hp6e20339_16188561_c1_41 | 2638 | 7400 |
| 04gp11213orf24 | 2639 | 7401 |
| 06cp30603_5351512_f3_73 | 2640 | 7402 |
| hpyhp3e10302orf_0011.aa | 2641 | 7403 |
| hpyhp3e10302orf_0010.aa | 2642 | 7404 |
| hpyhp3e10302orf_0007.aa | 2643 | 7405 |
| hp3e10302orf41 | 2644 | 7406 |
| hp3e10302orf37 | 2645 | 7407 |
| 06cp30603_7164010_c2_108 | 2646 | 7408 |
| hp6e20339_7241093_c1_42 | 2647 | 7409 |
| 04gp11213orf25 | 2648 | 7410 |
| 06cp30603_9781882_f1_17 | 2649 | 7411 |
| hp3e10302orf7 | 2650 | 7412 |
| 04ge10816_4032626_f2_9 | 2651 | 7413 |
| hpy12ae10622orf_0005.aa | 2652 | 7414 |
| 12ae10622orf15 | 2653 | 7415 |
| 12ae10622_16129812_c2_60 | 2654 | 7416 |
| 04ep71403orf11 | 2655 | 7417 |
| 04ep71403orf14 | 2656 | 7418 |
| 12ae10622_24305312_f1_8 | 2657 | 7419 |
| 12ge10321_26597180_f1_4 | 2658 | 7420 |
| 02ep20220orf9 | 2659 | 7421 |
| 06gp11920orf9 | 2660 | 7422 |
| 12ge10321_34094067_f2_12 | 2661 | 7423 |
| 12ae10622_34094067_f1_15 | 2662 | 7424 |
| 03ae10804_33782135_c3_44 | 2663 | 7425 |
| 06ep10306orf2 | 2664 | 7426 |
| 05cp10102orf3 | 2665 | 7427 |
| 12ge12015orf2 | 2666 | 7428 |
| hpy12ge12015orf_0001.aa | 2667 | 7429 |
| hp3e11188_12620840_f3_14 | 2668 | 7430 |
| 06ep11108orf19 | 2669 | 7431 |
| hp4e13394_4328343_f3_59 | 2670 | 7432 |
| hp5e13045_1464455_c1_8 | 2671 | 7433 |
| hp4e53394_1464455_c1_85 | 2672 | 7434 |
| 02ae11611orf3 | 2673 | 7435 |
| hp3e10975orf10 | 2674 | 7436 |
| hp3e10975orf5 | 2675 | 7437 |
| hp3e10975orf12 | 2676 | 7438 |
| hp4e53394_22005186_c1_82 | 2677 | 7439 |
| hp4e53394_22005186_c1_81 | 2678 | 7440 |
| hpyhp3e11182orf_0001.aa | 2679 | 7441 |
| 11cp10916orf1 | 2680 | 7442 |
| hpy11cp10916orf_0001.aa | 2681 | 7443 |
| hp4e53394_5864393_f2_39 | 2682 | 7444 |
| hp4p13376_38937_f3_3 | 2683 | 7445 |
| 05ce10208_1042133_c1_14 | 2684 | 7446 |
| hp1p13939orf11 | 2685 | 7447 |
| 14ae11614orf7 | 2686 | 7448 |
| 05ae11506orf1 | 2687 | 7449 |
| hpy05ae11506orf_0001.aa | 2688 | 7450 |
| 12gp11822orf3 | 2689 | 7451 |
| 12gp11822_25398512_c2_13 | 2690 | 7452 |
| hp1p13939_25398512_c1_26 | 2691 | 7453 |
| 14ae11614orf5 | 2692 | 7454 |
| hpy14ae11614orf_0005.aa | 2693 | 7455 |
| 14ae11614orf4 | 2694 | 7456 |
| 09cp71809orf3 | 2695 | 7457 |
| 12gp11822_4095312_c1_10 | 2696 | 7458 |
| hp3e11024orf4 | 2697 | 7459 |
| hp3e11024orf3 | 2698 | 7460 |
| 06ep30223_12538542_f2_58 | 2699 | 7461 |
| hp1e10506orf1 | 2700 | 7462 |
| 05cp21223_35710908_f2_11 | 2701 | 7463 |
| 07gp10713orf4 | 2702 | 7464 |
| hpy07gp10713orf_0001.aa | 2703 | 7465 |
| 06ep30223_1445250_f2_45 | 2704 | 7466 |
| 06ep30223_16512_c3_160 | 2705 | 7467 |
| hp3e10128orf1 | 2706 | 7468 |
| 02ae11612_26212767_c1_65 | 2707 | 7469 |
| 02ae11612orf40 | 2708 | 7470 |
| 16ep10117orf10 | 2709 | 7471 |
| 01ce11104_4689143_c2_13 | 2710 | 7472 |
| hp5e25328_4689143_f3_47 | 2711 | 7473 |
| hp5p15575orf10 | 2712 | 7474 |
| hp5p15575orf6 | 2713 | 7475 |
| hp5p15575_20037506_f3_19 | 2714 | 7476 |
| 04ee30405_6837792_f2_4 | 2715 | 7477 |
| hp5e15084orf12 | 2716 | 7478 |
| 01gp10401orf6 | 2717 | 7479 |
| hp6p10590_26181287_c1_27 | 2718 | 7480 |
| hp6p10590_338_c2_34 | 2719 | 7481 |
| 01gp10401orf3 | 2720 | 7482 |
| hp6p12244_23726552_f2_32 | 2721 | 7483 |
| hpy14cp10923orf_0001.aa | 2722 | 7484 |
| 13ee12016orf16 | 2723 | 7485 |
| hpy13ee12016orf_0001.aa | 2724 | 7486 |
| 13ee12016orf17 | 2725 | 7487 |
| 13ee12016orf9 | 2726 | 7488 |
| hp6p22217_24507800_f3_10 | 2727 | 7489 |
| hpy02ge10814orf_0001.aa | 2728 | 7490 |
| 11ae12004_24422692_f1_1 | 2729 | 7491 |
| 06ce20610orf9 | 2730 | 7492 |
| 11ae12004_14087833_f3_21 | 2731 | 7493 |
| 11ae12004orf1 | 2732 | 7494 |
| hpy11ae12004orf_0001.aa | 2733 | 7495 |
| 06ge10717orf3 | 2734 | 7496 |
| 06ce20610orf7 | 2735 | 7497 |
| 11ae12004_26384838_f2_6 | 2736 | 7498 |
| 06ce20610_26369057_c3_39 | 2737 | 7499 |
| hp7e10228_26369057_f1_5 | 2738 | 7500 |
| 13ae10712_3987531_f2_8 | 2739 | 7501 |
| 13ae10712orf6 | 2740 | 7502 |
| 07ge20415orf7 | 2741 | 7503 |
| 07ge20415orf16 | 2742 | 7504 |
| hp7e10429_4960880_f2_16 | 2743 | 7505 |
| hpy14ep11905orf_0006.aa | 2744 | 7506 |
| 14ep11905orf10 | 2745 | 7507 |
| hpy14ep11905orf_0008.aa | 2746 | 7508 |
| hpy14ep11905orf_0005.aa | 2747 | 7509 |
| 14ep11905orf7 | 2748 | 7510 |
| 14ce61516_1250_f1_7 | 2749 | 7511 |
| 14ce61516_1250_f1_7 | 2750 | 7512 |
| 14ep11905orf9 | 2751 | 7513 |
| hpy14ep11905orf_0001.aa | 2752 | 7514 |
| 14ep11905orf13 | 2753 | 7515 |
| 14ce61516_13073577_f2_12 | 2754 | 7516 |
| hp7e10590_13073577_c3_107 | 2755 | 7517 |
| 14ce61516_25402267_f1_8 | 2756 | 7518 |
| 14ce61516_12600937_f2_11 | 2757 | 7519 |
| 14ep11905orf8 | 2758 | 7520 |
| hpy14ep11905orf_0003.aa | 2759 | 7521 |
| 14cp11908_25402267_c3_104 | 2760 | 7522 |
| 04ap20904orf3 | 2761 | 7523 |
| 14ce21516orf1 | 2762 | 7524 |
| 14ce21516_85786_f1_1 | 2763 | 7525 |
| 07gp31516orf8 | 2764 | 7526 |
| 07gp31516orf1 | 2765 | 7527 |
| 09cp21607_33282767_c3_15 | 2766 | 7528 |
| 07gp31516orf2 | 2767 | 7529 |
| 07gp31516orf10 | 2768 | 7530 |
| 09cp21607_42950_c2_13 | 2769 | 7531 |
| hp7e30434_42950_f1_9 | 2770 | 7532 |
| hp7e10192_992337_f1_3 | 2771 | 7533 |
| 02ce10114orf2 | 2772 | 7534 |
| hpy02cp12005orf_0001.aa | 2773 | 7535 |
| hpy02cp12005orf_0002.aa | 2774 | 7536 |
| 05ae10702orf2 | 2775 | 7537 |
| 05ae10702orf1 | 2776 | 7538 |
| 05ge11017orf6 | 2777 | 7539 |
| 05ae10702orf4 | 2778 | 7540 |
| 05ge11017orf7 | 2779 | 7541 |
| 02cp12005orf4 | 2780 | 7542 |
| hpy02cp12005orf_0003.aa | 2781 | 7543 |
| 05ge11017orf5 | 2782 | 7544 |
| 06ae10902_13087518_f2_5 | 2783 | 7545 |
| hp2e11835orf2 | 2784 | 7546 |
| hp2p11834orf1 | 2785 | 7547 |
| 06ae10902_24306512_f2_3 | 2786 | 7548 |
| hp2p11834orf2 | 2787 | 7549 |
| 06ae10902_25394513_f1_1 | 2788 | 7550 |
| hp7p10325_24306512_f1_1 | 2789 | 7551 |
| 02ce41018_4960818_c3_41 | 2790 | 7552 |
| hp3p10349orf10 | 2791 | 7553 |
| 06ap20306_14455043_f3_8 | 2792 | 7554 |
| 04gp11803orf15 | 2793 | 7555 |
| 10677187 | 2794 | 7556 |
| 06ap11418_204202_f1_3 | 2795 | 7557 |
| hp5e15440orf18 | 2796 | 7558 |
| hpy03ae11503orf_0003.aa | 2797 | 7559 |
| 03ae11503orf6 | 2798 | 7560 |
| hpy03ae11503orf_0001.aa | 2799 | 7561 |
| 03ae11503orf11 | 2800 | 7562 |
| 03ae11503orf8 | 2801 | 7563 |
| 03ae11503_21602187_c1_16 | 2802 | 7564 |
| 07ap20216_33312808_f2_6 | 2803 | 7565 |
| 05ap21216orf1 | 2804 | 7566 |
| hp3p10343orf2 | 2805 | 7567 |
| hpyhp3p10343orf_0001.aa | 2806 | 7568 |
| 11ap20714_33376087_f2_25 | 2807 | 7569 |
| 12520952 | 9605 | 9717 |
| 487750 | 9606 | 9718 |
| 85786 | 9607 | 9719 |
| 36523442 | 9608 | 9720 |
| B.9 Proteins involved in secretion and | | |
| adhesion | | |
| hp6e12267_14642202_c2_47 | 2808 | 7570 |
| 12ge10305orf15 | 2809 | 7571 |
| 14642202 | 2810 | 7572 |
| 30662792 | 2811 | 7573 |
| 14cp10119orf16 | 2812 | 7574 |
| 34427317 | 2813 | 7575 |
| 14cp10119orf15 | 2814 | 7576 |
| 14cp10119orf12 | 2815 | 7577 |
| 04cp11202_13781712_f2_27 | 2816 | 7578 |
| 02cp11404orf1 | 2817 | 7579 |
| 6517192 | 2818 | 7580 |
| 02cp11404orf9 | 2819 | 7581 |
| 07ge11521_21494062_c1_27 | 2820 | 7582 |
| 03ae11503orf9 | 2821 | 7583 |
| hp3e10057orf5 | 2822 | 7584 |
| hpyhp3e10057orf_0004.aa | 2823 | 7585 |
| 03ae11503_24881306_c2_18 | 2824 | 7586 |
| B.10 Cytoplasmic proteins involved in outer | | |
| membrane & cell wall biogenesis | | |
| hpy11gp10904orf_0002.aa | 2825 | 7587 |
| 14713512 | 2826 | 7588 |
| 11gp10904orf29 | 2827 | 7589 |
| 11gp20904_14713512_c1_27 | 2828 | 7590 |
| hp5e15211_11884425_c2_27 | 2829 | 7591 |
| hp5e15211orf32 | 2830 | 7592 |
| 4578469 | 2831 | 7593 |
| hp5e15211_4578467_c2_23 | 2832 | 7594 |
| hp5e15211orf22 | 2833 | 7595 |
| 04ee11108_23486342_c2_20 | 2834 | 7596 |
| 23486342 | 2835 | 7597 |
| 27ze10351orf22 | 2836 | 7598 |
| hpy02ap21113orf_0003.aa | 2837 | 7599 |
| hpy11ep12011orf_0003.aa | 2838 | 7600 |
| 02ap21113orf1 | 2839 | 7601 |
| hpy11ep12011orf_0004.aa | 2840 | 7602 |
| 06gp71906_1257700_c1_124 | 2841 | 7603 |
| 04ep41903_14729692_f3_15 | 2842 | 7604 |
| 29ap10306orf2 | 2843 | 7605 |
| 11253 | 2844 | 7606 |
| 09ce52017_25890837_c2_24 | 2845 | 7607 |
| 01ep30520orf27 | 2846 | 7608 |
| 09cp10224orf6 | 2847 | 7609 |
| 09cp10224orf3 | 2848 | 7610 |
| 09cp10224orf7 | 2849 | 7611 |
| 09cp10224_34180312_c3_64 | 2850 | 7612 |
| 06cp10117orf1 | 2851 | 7613 |
| hpy06cp10117orf_0001.aa | 2852 | 7614 |
| 09cp10224_4578292_c3_62 | 2853 | 7615 |
| 12ae10622_5192075_f2_17 | 2854 | 7616 |
| 12ae10622orf8 | 2855 | 7617 |
| hpy12ae10622orf_0001.aa | 2856 | 7618 |
| 05gp10919_11148454_c2_2 | 2857 | 7619 |
| 05gp10919orf1 | 2858 | 7620 |
| hpy05gp10919orf_0001.aa | 2859 | 7621 |
| hp1e80523_1208413_f1_3 | 2860 | 7622 |
| 02ae11612orf43 | 2861 | 7623 |
| 02ae11612_12695250_c3_99 | 2862 | 7624 |
| 05cp11911orf33 | 2863 | 7625 |
| 02ae11612_24257818_c2_72 | 2864 | 7626 |
| 06ep11917_4563939_c1_19 | 2865 | 7627 |
| hp5e25328_5084843_f1_20 | 2866 | 7628 |
| 429192 | 2867 | 7629 |
| hp6p10509_24304675_c1_13 | 2868 | 7630 |
| 16ae10508orf21 | 2869 | 7631 |
| hpy13ee12016orf_0004.aa | 2870 | 7632 |
| 30082267 | 2871 | 7633 |
| 13ee12016orf10 | 2872 | 7634 |
| hp6p22217_33398377_f3_11 | 2873 | 7635 |
| hp7e10590_14162751_c1_77 | 2874 | 7636 |
| 23535937 | 2875 | 7637 |
| 06cp11722orf15 | 2876 | 7638 |
| 11ae80818_3322192_f3_37 | 2877 | 7639 |
| 24441412 | 9609 | 9721 |
| 34265691 | 9610 | 9722 |
| B.11 Cytoplasmic proteins involved in | | |
| protein folding and stabilization | | |
| hpy05ap11821orf_0001.aa | 2878 | 7640 |
| 11ae11815orf1 | 2879 | 7641 |
| hpy11ae11815orf_0001.aa | 2880 | 7642 |
| 05ap11821orf4 | 2881 | 7643 |
| hpy05ap11821orf_0002.aa | 2882 | 7644 |
| 09cp61003_4879443_c1_60 | 2883 | 7645 |
| hp5e12982orf16 | 2884 | 7646 |
| hp5e12982orf13 | 2885 | 7647 |
| hpyhp5e12982orf_0001.aa | 2886 | 7648 |
| hp5e12982_32455418_c1_15 | 2887 | 7649 |
| hp6p21623_32455418_f2_11 | 2888 | 7650 |
| 05gp11602orf1 | 2889 | 7651 |
| 06ap30322orf10 | 2890 | 7652 |
| hpy06ap30322orf_0004.aa | 2891 | 7653 |
| 06ap30322_24509666_c2_9 | 2892 | 7654 |
| 06ap30322_4727318_c3_32 | 2893 | 7655 |
| hp1p13947orf8 | 2894 | 7656 |
| 29gp10716orf1 | 2895 | 7657 |
| hp1p13947orf1 | 2896 | 7658 |
| hpyhp1p13947orf_0001.aa | 2897 | 7659 |
| 02ae31010_24408387_f3_35 | 2898 | 7660 |
| hp6p10904_260887_c1_19 | 2899 | 7661 |
| hp4p13446orf2 | 2900 | 7662 |
| hp4p13446orf8 | 2901 | 7663 |
| 14ep10823orf4 | 2902 | 7664 |
| hpy14ep10823orf_0001.aa | 2903 | 7665 |
| hp6p10904_260887_c2_20 | 2904 | 7666 |
| 09cp10224_24220313_c1_44 | 2905 | 7667 |
| hpy09cp10224orf_0002.aa | 2906 | 7668 |
| 09cp10224_3932187_c1_45 | 2907 | 7669 |
| hp3e11024orf20 | 2908 | 7670 |
| hp3e11024orf25 | 2909 | 7671 |
| 06ep30223_31449217_f3_97 | 2910 | 7672 |
| 13ae10712_4031500_f3_15 | 2911 | 7673 |
| 13ae10712orf8 | 2912 | 7674 |
| 11ae80818_4500342_f2_17 | 2913 | 7675 |
| 06cp11722orf17 | 2914 | 7676 |
| 09cp21607_34647812_c1_8 | 2915 | 7677 |
| 07gp31516orf5 | 2916 | 7678 |
| 05ce10910_23712780_c1_4 | 2917 | 7679 |
| hp7e10192_23712780_f2_5 | 2918 | 7680 |
| 12343763 | 9611 | 9723 |
| 6845425 | 9612 | 9724 |
| B.12 Other cytoplasmic proteins | | |
| hp7p10290_1378917_f3_13 | 2919 | 7681 |
| hp3p10156orf5 | 2920 | 7682 |
| hp6e12267_2038152_c1_40 | 2921 | 7683 |
| 12ge20305orf1 | 2922 | 7684 |
| 09cp61003_23861502_f2_38 | 2923 | 7685 |
| 11ee10423orf3 | 2924 | 7686 |
| hpy11ee10423orf_0001.aa | 2925 | 7687 |
| hp4p12360orf2 | 2926 | 7688 |
| 09cp61003_25546943_c3_110 | 2927 | 7689 |
| hpy01ce11016orf_0004.aa | 2928 | 7690 |
| hpy01ce11016orf_0005.aa | 2929 | 7691 |
| hp6p80503_32462717_f1_5 | 2930 | 7692 |
| 11cp12002orf3 | 2931 | 7693 |
| 11cp12002orf2 | 2932 | 7694 |
| hp7p10290_35156558_f3_15 | 2933 | 7695 |
| hp6p80503_35742151_f3_15 | 2934 | 7696 |
| 11cp11224orf1 | 2935 | 7697 |
| hp6e12267_4095342_f1_4 | 2936 | 7698 |
| 12ge20305orf21 | 2937 | 7699 |
| hp3p10156orf7 | 2938 | 7700 |
| hp7p10290_4351718_f1_6 | 2939 | 7701 |
| 03gp10711orf2 | 2940 | 7702 |
| hpy03gp10711orf_0001.aa | 2941 | 7703 |
| 09cp61003_4414068_c2_95 | 2942 | 7704 |
| hp7p10290_4531713_f2_10 | 2943 | 7705 |
| hp3p10156orf1 | 2944 | 7706 |
| hp6e12267_4570263_f2_24 | 2945 | 7707 |
| 12ge10305orf12 | 2946 | 7708 |
| 01ge11619_1962500_c2_10 | 2947 | 7709 |
| 14ap11617_32031525_c1_9 | 2948 | 7710 |
| 01cp10108_1962500_c3_8 | 2949 | 7711 |
| hpy05ap11505orf_0003.aa | 2950 | 7712 |
| 11ge10309orf3 | 2951 | 7713 |
| hp6p21623_22843812_c2_52 | 2952 | 7714 |
| hpy09ap20802orf_0020.aa | 2953 | 7715 |
| hp6p21623_24476562_f3_25 | 2954 | 7716 |
| hp5e12982_26362717_f1_1 | 2955 | 7717 |
| hp5e12982orf4 | 2956 | 7718 |
| hp6p21623_33475205_f3_23 | 2957 | 7719 |
| hpyhp5e12982orf_0003.aa | 2958 | 7720 |
| hp6p21623_34172075_c2_54 | 2959 | 7721 |
| hpy09ap20802orf_0015.aa | 2960 | 7722 |
| hp6p21623_34833_c1_40 | 2961 | 7723 |
| hpy09ap20802orf_0011.aa | 2962 | 7724 |
| hp6p21623_35162518_f2_10 | 2963 | 7725 |
| hpyhp5e12982orf_0004.aa | 2964 | 7726 |
| 01ae10202_4710925_f1_2 | 2965 | 7727 |
| 11ge10309orf30 | 2966 | 7728 |
| 04ap12016_4725311_c1_5 | 2967 | 7729 |
| 29ep10720_4725311_f3_21 | 2968 | 7730 |
| 02ap21102_4725311_f2_15 | 2969 | 7731 |
| 11ge10309orf21 | 2970 | 7732 |
| 02gp20706_3336468_c3_61 | 2971 | 7733 |
| 02ge10116_3336468_c2_109 | 2972 | 7734 |
| 06ge10115orf9 | 2973 | 7735 |
| 02ge10116_10756328_c1_93 | 2974 | 7736 |
| hp6p12311_24240708_c1_7 | 2975 | 7737 |
| 05ep20322orf9 | 2976 | 7738 |
| 02ge10116_12307812_f3_71 | 2977 | 7739 |
| 02ge10116_12505125_c3_31 | 2978 | 7740 |
| 12505125 | 2979 | 7741 |
| 02ge20116orf20 | 2980 | 7742 |
| 02ge10116_1376592_f2_50 | 2981 | 7743 |
| 05ep20322orf7 | 2982 | 7744 |
| 02ge10116_14182328_f2_29 | 2983 | 7745 |
| 02ge20116orf2 | 2984 | 7746 |
| 02gp20706_14507827_c3_65 | 2985 | 7747 |
| 01cp11710orf38 | 2986 | 7748 |
| 02ge10116_14649212_c1_20 | 2987 | 7749 |
| 02ge20116orf26 | 2988 | 7750 |
| hpy11ae10305orf_0002.aa | 2989 | 7751 |
| 02ge10116_19667752_f3_68 | 2990 | 7752 |
| hpy05ee20322orf_0004.aa | 2991 | 7753 |
| hpy05ee20322orf_0002.aa | 2992 | 7754 |
| 02ge10116_22854818_f2_47 | 2993 | 7755 |
| 02ge10116_23650308_f1_24 | 2994 | 7756 |
| hpyhp5e15276orf_0003.aa | 2995 | 7757 |
| 24070250 | 2996 | 7758 |
| 02gp20706_24070250_c1_42 | 2997 | 7759 |
| 02ge10116_24070250_c3_150 | 2998 | 7760 |
| 06ge10115orf15 | 2999 | 7761 |
| 02ge10116_26431543_f2_8 | 3000 | 7762 |
| 02ge20116orf1 | 3001 | 7763 |
| 02ge10116_31798312_c1_94 | 3002 | 7764 |
| hpy02ge20116orf_0011.aa | 3003 | 7765 |
| hp5e15276orf15 | 3004 | 7766 |
| 02ge10116_33313816_f3_67 | 3005 | 7767 |
| 02ge10116_36230312_c2_114 | 3006 | 7768 |
| hpy01cp11710orf_0013.aa | 3007 | 7769 |
| 02ge20116orf7 | 3008 | 7770 |
| 02ge20116orf13 | 3009 | 7771 |
| 02ge10116_36369175_f1_1 | 3010 | 7772 |
| hpy05ep20322orf_0011.aa | 3011 | 7773 |
| 05ep20322orf12 | 3012 | 7774 |
| 05ep20322orf13 | 3013 | 7775 |
| hp6p12311_4689000_f2_3 | 3014 | 7776 |
| 02ge10116_4689000_c3_130 | 3015 | 7777 |
| 02gp20706_4767967_f3_21 | 3016 | 7778 |
| 01cp11710orf3 | 3017 | 7779 |
| 02ge10116_797180_c2_118 | 3018 | 7780 |
| hpy01cp11710orf_0011.aa | 3019 | 7781 |
| 02ge10116_977183_f3_52 | 3020 | 7782 |
| hpy02ge20116orf_0005.aa | 3021 | 7783 |
| 02ge10116_9899142_f3_14 | 3022 | 7784 |
| 02ge20116orf8 | 3023 | 7785 |
| 02gp20706_12303811_c3_59 | 3024 | 7786 |
| 02gp20706_992200_c1_38 | 3025 | 7787 |
| 06ge10115orf11 | 3026 | 7788 |
| 06ge10115orf7 | 3027 | 7789 |
| 02ge10116_992200_c2_107 | 3028 | 7790 |
| hp6p12311_9928957_c2_10 | 3029 | 7791 |
| 02ge10116_9928957_f1_26 | 3030 | 7792 |
| 05ep20322orf1 | 3031 | 7793 |
| 02ge11810orf1 | 3032 | 7794 |
| hpy02ge11810orf_0001.aa | 3033 | 7795 |
| 02ge11810_1457812_f1_1 | 3034 | 7796 |
| 04ap10921orf4 | 3035 | 7797 |
| 04ap10921orf6 | 3036 | 7798 |
| 04ap10921orf1 | 3037 | 7799 |
| 02ge11810_4532768_f1_2 | 3038 | 7800 |
| 02ge41622_212825_f1_2 | 3039 | 7801 |
| hpy13ee10216orf_0005.aa | 3040 | 7802 |
| 13ee10216orf20 | 3041 | 7803 |
| 13ee10216orf40 | 3042 | 7804 |
| 02ge11622_23469167_f2_17 | 3043 | 7805 |
| 02ge11622_24401536_f1_3 | 3044 | 7806 |
| 13ee10216orf3 | 3045 | 7807 |
| 03ce10801orf2 | 3046 | 7808 |
| hpy06ee10709orf_0009.aa | 3047 | 7809 |
| 02ep30607orf4 | 3048 | 7810 |
| hpy05ap10120orf_0004.aa | 3049 | 7811 |
| 06ee30709_23573392_c3_32 | 3050 | 7812 |
| 02ge41622_2926257_c3_96 | 3051 | 7813 |
| 13ee10216orf36 | 3052 | 7814 |
| 13ee10216orf35 | 3053 | 7815 |
| 02ge11622_4296877_f3_32 | 3054 | 7816 |
| 02ge11622_5884392_f2_14 | 3055 | 7817 |
| 13ee10216orf16 | 3056 | 7818 |
| 05ae30220_29458178_f3_47 | 3057 | 7819 |
| 29458178 | 3058 | 7820 |
| 05ae20220orf51 | 3059 | 7821 |
| 05ae30220_36132687_f1_4 | 3060 | 7822 |
| 05ae20220orf4 | 3061 | 7823 |
| 04ap20412_5250252_c3_1 | 3062 | 7824 |
| hp1p10543orf6 | 3063 | 7825 |
| 07ge11504orf5 | 3064 | 7826 |
| 07ge11504orf1 | 3065 | 7827 |
| hpy07ge11504orf_0001.aa | 3066 | 7828 |
| 05ep10815_10972587_c1_85 | 3067 | 7829 |
| 05ep10815_14273452_c3_105 | 3068 | 7830 |
| 01xe21717orf3 | 3069 | 7831 |
| 05ep10815_14646876_f3_63 | 3070 | 7832 |
| hpy01xe21717orf_0019.aa | 3071 | 7833 |
| 11gp20904_22447333_f1_8 | 3072 | 7834 |
| 03ce11207_15041083_f1_44 | 3073 | 7835 |
| 11gp10904orf5 | 3074 | 7836 |
| 01cp20708_15625202_c3_60 | 3075 | 7837 |
| hpy14ee10419orf_0008.aa | 3076 | 7838 |
| 11gp20904_23442160_f2_11 | 3077 | 7839 |
| 11gp10904orf11 | 3078 | 7840 |
| hpy06ae11405orf_0001.aa | 3079 | 7841 |
| hpy06ae11405orf_0003.aa | 3080 | 7842 |
| 01xe21717orf15 | 3081 | 7843 |
| hpy01xe21717orf_0001.aa | 3082 | 7844 |
| hpy06ae11405orf_0002.aa | 3083 | 7845 |
| 05ep10815_23845162_c1_75 | 3084 | 7846 |
| 06ep10822orf5 | 3085 | 7847 |
| hp4e14522orf2 | 3086 | 7848 |
| 01ae11010_23854175_f2_15 | 3087 | 7849 |
| 01ae11010_23854175_f1_4 | 3088 | 7850 |
| 01ae11010_23854175_f2_16 | 3089 | 7851 |
| 02ep10409_23992002_f2_4 | 3090 | 7852 |
| 01ae11010_23992002_f3_30 | 3091 | 7853 |
| 07cp10312orf1 | 3092 | 7854 |
| hpy09ge10522orf_0001.aa | 3093 | 7855 |
| 09ge10522orf1 | 3094 | 7856 |
| 05ep10815_24042550_f3_60 | 3095 | 7857 |
| 13ap10114orf1 | 3096 | 7858 |
| 01cp20708_24235943_f1_9 | 3097 | 7859 |
| 05ep10815_24406532_f3_64 | 3098 | 7860 |
| hpy01xe21717orf_0018.aa | 3099 | 7861 |
| 11ap21101_23441308_f2_2 | 3100 | 7862 |
| 01ae11010_26179686_f2_11 | 3101 | 7863 |
| hpy07cp10312orf_0004.aa | 3102 | 7864 |
| 01ce10320_26353437_f3_9 | 3103 | 7865 |
| 01ce10320orf5 | 3104 | 7866 |
| 05ep10815_26460805_c1_76 | 3105 | 7867 |
| 01xe21717orf10 | 3106 | 7868 |
| 29844512 | 3107 | 7869 |
| 11gp20904_30705212_f2_12 | 3108 | 7870 |
| 11gp10904orf12 | 3109 | 7871 |
| 03ae10522orf1 | 3110 | 7872 |
| hpy03ae10522orf_0001.aa | 3111 | 7873 |
| 05ep10815_32140957_c3_108 | 3112 | 7874 |
| 03ce11207_32140957_c2_233 | 3113 | 7875 |
| hpy01xe21717orf_0014.aa | 3114 | 7876 |
| 01xe21717orf43 | 3115 | 7877 |
| 05ep10815_33237561_f3_66 | 3116 | 7878 |
| hpy01xe21717orf_0002.aa | 3117 | 7879 |
| 01xe21717orf16 | 3118 | 7880 |
| 05ep10815_34270136_c3_102 | 3119 | 7881 |
| 156587 | 3120 | 7882 |
| 05ep10815_34649002_c2_93 | 3121 | 7883 |
| 01xe21717orf9 | 3122 | 7884 |
| 01ae11010_35945827_c1_41 | 3123 | 7885 |
| hpyhp4e14522orf_0007.aa | 3124 | 7886 |
| hp3e11170orf3 | 3125 | 7887 |
| hp3e11170orf2 | 3126 | 7888 |
| hp3e11170orf1 | 3127 | 7889 |
| hpyhp3e11170orf_0001.aa | 3128 | 7890 |
| 03ce11207orf1 | 3129 | 7891 |
| 01cp20708_35995718_c3_55 | 3130 | 7892 |
| hp3e11168orf28 | 3131 | 7893 |
| 04gp11701_36114702_c1_24 | 3132 | 7894 |
| 05ep10815_3949067_c2_91 | 3133 | 7895 |
| 06ae11405orf7 | 3134 | 7896 |
| hpyhp3p10960orf_0001.aa | 3135 | 7897 |
| 14ae21813orf1 | 3136 | 7898 |
| hpy14ae21813orf_0001.aa | 3137 | 7899 |
| 05ep10815_397937_c3_107 | 3138 | 7900 |
| 01ae11010_42813_f2_10 | 3139 | 7901 |
| 02ep10409_42813_f1_1 | 3140 | 7902 |
| hpy07cp10312orf_0003.aa | 3141 | 7903 |
| 01ae11010_5103403_c1_42 | 3142 | 7904 |
| 01ae11010_5103403_c3_34 | 3143 | 7905 |
| hp4e14522orf13 | 3144 | 7906 |
| 01cp20708_5289051_f3_29 | 3145 | 7907 |
| 14ee10419orf2 | 3146 | 7908 |
| hpy06ae11405orf_0007.aa | 3147 | 7909 |
| 06ae11405orf9 | 3148 | 7910 |
| 05ep10815_24414587_c1_74 | 3149 | 7911 |
| 03ce11207_5314018_c3_268 | 3150 | 7912 |
| 01ae11010_5314202_f1_1 | 3151 | 7913 |
| 07cp10312orf6 | 3152 | 7914 |
| 01ae11010_26620878_c2_48 | 3153 | 7915 |
| 29ee12019orf2 | 3154 | 7916 |
| hpy29ee12019orf_0001.aa | 3155 | 7917 |
| 03ce11207_6525337_f2_101 | 3156 | 7918 |
| 03cp11207orf1 | 3157 | 7919 |
| 01cp20708_7070967_c2_48 | 3158 | 7920 |
| 06ep11502orf1 | 3159 | 7921 |
| hpy06ep11502orf_0001.aa | 3160 | 7922 |
| hp5e15211_4494659_c2_26 | 3161 | 7923 |
| 03gp11902_23473457_f3_12 | 3162 | 7924 |
| hp5e15211_35163962_f1_3 | 3163 | 7925 |
| 35163962 | 3164 | 7926 |
| hp5e15211_35163962_f1_3 | 3165 | 7927 |
| hp5e15211orf13 | 3166 | 7928 |
| hp5e15211_994043_c1_24 | 3167 | 7929 |
| hpyhp5e15211orf_0003.aa | 3168 | 7930 |
| 01ae11612_16680312_f2_7 | 3169 | 7931 |
| 07ep11110orf4 | 3170 | 7932 |
| 01ae11612_22948342_f3_9 | 3171 | 7933 |
| 07ep11110orf1 | 3172 | 7934 |
| 01ae11612_30601577_f1_3 | 3173 | 7935 |
| 07ep11110orf3 | 3174 | 7936 |
| 29ep10720_23442127_f1_2 | 3175 | 7937 |
| 06ap10920_33339057_f1_1 | 3176 | 7938 |
| 11ge10309orf31 | 3177 | 7939 |
| 07ep11110orf5 | 3178 | 7940 |
| 01ae11612_4001563_f2_8 | 3179 | 7941 |
| hp5e15717_21569141_c1_1 | 3180 | 7942 |
| hpy06gp10108orf_0002.aa | 3181 | 7943 |
| 02ce10809orf3 | 3182 | 7944 |
| 02ce10809orf7 | 3183 | 7945 |
| 14ce31519_236087_f1_5 | 3184 | 7946 |
| hp5e15717_24319005_c2_2 | 3185 | 7947 |
| hpy06gp10108orf_0001.aa | 3186 | 7948 |
| 04ep41903_24319005_c2_57 | 3187 | 7949 |
| 02cp10815orf3 | 3188 | 7950 |
| hpy02cp10815orf_0002.aa | 3189 | 7951 |
| 11cp11617orf1 | 3190 | 7952 |
| 04ep41903_24423963_f1_3 | 3191 | 7953 |
| 05ae30220_127_c3_177 | 3192 | 7954 |
| hpy05ae20220orf_0026.aa | 3193 | 7955 |
| 05ae30220_1360633_c3_100 | 3194 | 7956 |
| 05ae20220orf97 | 3195 | 7957 |
| hpy05ap10120orf_0002.aa | 3196 | 7958 |
| 03ce10801orf3 | 3197 | 7959 |
| hpy05ap10120orf_0003.aa | 3198 | 7960 |
| 05ae30220_16519662_c2_128 | 3199 | 7961 |
| 05ae30220_16519662_c3_204 | 3200 | 7962 |
| 05ae30220_16523587_f3_69 | 3201 | 7963 |
| hpy05ae20220orf_0019.aa | 3202 | 7964 |
| 05ae30220_16687512_c1_66 | 3203 | 7965 |
| 05ae20220orf118 | 3204 | 7966 |
| 05ae30220_22546878_c3_171 | 3205 | 7967 |
| 05ae30220_23438156_c2_137 | 3206 | 7968 |
| 05ae30220_23636252_f2_30 | 3207 | 7969 |
| 05ae20220orf9 | 3208 | 7970 |
| 05ae30220_23945442_f3_48 | 3209 | 7971 |
| 05ae20220orf27 | 3210 | 7972 |
| 05ae30220_24297042_f3_94 | 3211 | 7973 |
| 27ze10351orf2 | 3212 | 7974 |
| 05ae30220_24812811_c1_67 | 3213 | 7975 |
| 05ae20220orf75 | 3214 | 7976 |
| 05ae30220_25400312_c2_84 | 3215 | 7977 |
| 05ae20220orf73 | 3216 | 7978 |
| 11ee11408_25431467_c2_44 | 3217 | 7979 |
| hp5p15496orf4 | 3218 | 7980 |
| 05ae30220_25587788_f2_29 | 3219 | 7981 |
| 05ae20220orf8 | 3220 | 7982 |
| 05ae30220_25656312_c3_102 | 3221 | 7983 |
| 05ae20220orf99 | 3222 | 7984 |
| 05ae30220_3240902_f1_11 | 3223 | 7985 |
| 05ae20220orf13 | 3224 | 7986 |
| 05ae30220_34022813_c3_185 | 3225 | 7987 |
| 05ae30220_24492040_f1_2 | 3226 | 7988 |
| 05ae20220orf2 | 3227 | 7989 |
| 05ae30220_36618786_f1_2 | 3228 | 7990 |
| hpy05ae20220orf_0003.aa | 3229 | 7991 |
| 05ae30220_34198517_f2_48 | 3230 | 7992 |
| 07ap80601_4304687_f2_7 | 3231 | 7993 |
| 07ap80601orf6 | 3232 | 7994 |
| 07ap80601orf13 | 3233 | 7995 |
| 07ap80601_36023958_f3_11 | 3234 | 7996 |
| 05ae30220_4304687_c1_109 | 3235 | 7997 |
| 04ee11108_4695468_f3_12 | 3236 | 7998 |
| 27ze10351orf12 | 3237 | 7999 |
| hp5p15496orf6 | 3238 | 8000 |
| 11ee11408_4881693_c3_53 | 3239 | 8001 |
| 05ae30220_5283127_f1_10 | 3240 | 8002 |
| 05ae20220orf12 | 3241 | 8003 |
| 05ae30220_7265638_f2_35 | 3242 | 8004 |
| 05ae20220orf38 | 3243 | 8005 |
| 05ae30220_15719657_f1_1 | 3244 | 8006 |
| 05ae30220_9881408_f1_1 | 3245 | 8007 |
| 05ae20220orf1 | 3246 | 8008 |
| 05ae30220_23551075_f3_71 | 3247 | 8009 |
| 05ae30801_11753308_f3_13 | 3248 | 8010 |
| 05ae30801_13869058_c3_26 | 3249 | 8011 |
| 13ap11204orf6 | 3250 | 8012 |
| hp5p15212_13883512_f1_1 | 3251 | 8013 |
| 02ce10213orf13 | 3252 | 8014 |
| 14640637 | 3253 | 8015 |
| hp5p15212_14641462_f3_14 | 3254 | 8016 |
| 02ce10213orf15 | 3255 | 8017 |
| hp5p15212_23691892_f2_6 | 3256 | 8018 |
| 02ce10213orf9 | 3257 | 8019 |
| hp5p15212_30173405_f3_15 | 3258 | 8020 |
| 02ce10213orf16 | 3259 | 8021 |
| 02ce10213orf8 | 3260 | 8022 |
| 02ce10213orf14 | 3261 | 8023 |
| hp5p15212_34172000_f3_13 | 3262 | 8024 |
| 02ce10213orf17 | 3263 | 8025 |
| 02ce10213orf1 | 3264 | 8026 |
| 4531568 | 3265 | 8027 |
| hp5p15212_4531568_f3_16 | 3266 | 8028 |
| 05ee11015orf1 | 3267 | 8029 |
| 12ae11404_29307332_f3_5 | 3268 | 8030 |
| 12ae11404orf2 | 3269 | 8031 |
| hpy12ae11404orf_0001.aa | 3270 | 8032 |
| 05ee11015_13801386_f3_5 | 3271 | 8033 |
| 12ae11404_24414087_f1_1 | 3272 | 8034 |
| 12ae11404orf7 | 3273 | 8035 |
| 09ae10512_12692805_f3_27 | 3274 | 8036 |
| hp3e11060orf13 | 3275 | 8037 |
| 09ae10512_14329791_f3_16 | 3276 | 8038 |
| 07ee20513orf2 | 3277 | 8039 |
| 06ae11016_23438577_f2_18 | 3278 | 8040 |
| hpyhp3e11060orf_0009.aa | 3279 | 8041 |
| 24220627 | 3280 | 8042 |
| 09ae10512_24220627_f1_3 | 3281 | 8043 |
| 07ee20513orf14 | 3282 | 8044 |
| 09ae10512_34023462_f3_26 | 3283 | 8045 |
| hp3e11060orf12 | 3284 | 8046 |
| 07ee20513orf21 | 3285 | 8047 |
| hpy07ee20513orf_0010.aa | 3286 | 8048 |
| 09ae10512_54187_c2_53 | 3287 | 8049 |
| 09ae10512_579425_c2_51 | 3288 | 8050 |
| hp3e11060orf4 | 3289 | 8051 |
| hp3e11060orf11 | 3290 | 8052 |
| hpyhp3e11060orf_0012.aa | 3291 | 8053 |
| 09ae10512_21642642_c1_42 | 3292 | 8054 |
| 06ae11016_579425_c2_53 | 3293 | 8055 |
| 06ae11414_7627_f1_1 | 3294 | 8056 |
| hpy11ge10309orf_0033.aa | 3295 | 8057 |
| 06ap30322_3956503_c3_34 | 3296 | 8058 |
| 06ap30322orf11 | 3297 | 8059 |
| 06ap10322_3956503_c2_34 | 3298 | 8060 |
| 06ap30322_4415912_f3_23 | 3299 | 8061 |
| 03ae10516orf3 | 3300 | 8062 |
| hpy03ae10516orf_0002.aa | 3301 | 8063 |
| hpy03ae10516orf_0003.aa | 3302 | 8064 |
| 06ap30322_781410_f2_13 | 3303 | 8065 |
| 07gp11807orf38 | 3304 | 8066 |
| 07gp11807orf51 | 3305 | 8067 |
| 01ae12001_214812_c3_49 | 3306 | 8068 |
| 35949212 | 3307 | 8069 |
| 01ae12001_35949212_c1_36 | 3308 | 8070 |
| 07gp11807orf44 | 3309 | 8071 |
| 01ae12001_4882842_c2_44 | 3310 | 8072 |
| 07gp11807orf33 | 3311 | 8073 |
| 01ae12001_3335908_c3_51 | 3312 | 8074 |
| 07gp11807orf41 | 3313 | 8075 |
| 06ap10609_4882842_c3_54 | 3314 | 8076 |
| 4882842 | 3315 | 8077 |
| 07gp11807orf36 | 3316 | 8078 |
| 14ge10814orf2 | 3317 | 8079 |
| hpy14ge10814orf_0003.aa | 3318 | 8080 |
| 01ae12001orf1 | 3319 | 8081 |
| 06ae10609orf1 | 3320 | 8082 |
| 01ae12001_1070250_c3_55 | 3321 | 8083 |
| 06ap10609_78527_c3_58 | 3322 | 8084 |
| 06ap10609_1070250_c1_41 | 3323 | 8085 |
| 21647676 | 3324 | 8086 |
| 06ap11119_11892135_f2_16 | 3325 | 8087 |
| 11ge10309orf15 | 3326 | 8088 |
| 06ap11119_14253937_c2_42 | 3327 | 8089 |
| hpyhp6p10723orf_0021.aa | 3328 | 8090 |
| hpy01ce11016orf_0014.aa | 3329 | 8091 |
| 06ap11119_14641700_f2_15 | 3330 | 8092 |
| hp6p10723orf21 | 3331 | 8093 |
| 01ae10202_24095452_f3_3 | 3332 | 8094 |
| 11ge10309orf16 | 3333 | 8095 |
| 06ap11119_33205290_f2_12 | 3334 | 8096 |
| hp6p1 0723orf18 | 3335 | 8097 |
| 06ap11119_4879713_f3_24 | 3336 | 8098 |
| 11ge10309orf26 | 3337 | 8099 |
| 06ap11119_5112776_c2_43 | 3338 | 8100 |
| hpyhp6p10723orf_0020.aa | 3339 | 8101 |
| 06ap11119_9797877_f2_13 | 3340 | 8102 |
| hp6p10723orf19 | 3341 | 8103 |
| 12gp11615_4141092_c2_5 | 3342 | 8104 |
| 02ap21102_4141092_c3_39 | 3343 | 8105 |
| hpy11ge10309orf_0002.aa | 3344 | 8106 |
| hp6e10967_19728428_f2_2 | 3345 | 8107 |
| hp2p10625orf1 | 3346 | 8108 |
| 05ee10816_236093_f3_17 | 3347 | 8109 |
| 04ce11617orf3 | 3348 | 8110 |
| 06ep10615_23866311_f1_23 | 3349 | 8111 |
| 06ce10311orf2 | 3350 | 8112 |
| 06ep10615_24410965_c3_104 | 3351 | 8113 |
| 06ep10615_32617012_c3_91 | 3352 | 8114 |
| hpyhp2p10625orf_0006.aa | 3353 | 8115 |
| 13ee10424orf1 | 3354 | 8116 |
| hpy13ee10424orf_0001.aa | 3355 | 8117 |
| 06ep10615_33416562_c2_82 | 3356 | 8118 |
| hpy05ep11717orf_0001.aa | 3357 | 8119 |
| hpy05ep11717orf_0003.aa | 3358 | 8120 |
| hpy05ep11717orf_0006.aa | 3359 | 8121 |
| hpy05ep11717orf_0005.aa | 3360 | 8122 |
| 05ep11717orf7 | 3361 | 8123 |
| 05ep11717orf6 | 3362 | 8124 |
| 06ep10615_35943787_c2_81 | 3363 | 8125 |
| 06ep11202_2773375_f3_13 | 3364 | 8126 |
| hpy06cp20302orf_0009.aa | 3365 | 8127 |
| 06ep11202_4531312_c1_23 | 3366 | 8128 |
| hpyhp2e11858orf_0002.aa | 3367 | 8129 |
| hp3e11122orf2 | 3413 | 8175 |
| 06gp71906_4494063_c2_154 | 3414 | 8176 |
| 06gp71906_5214068_f1_28 | 3415 | 8177 |
| 12gp11106orf10 | 3416 | 8178 |
| 12gp31106_6835827_f1_11 | 3417 | 8179 |
| 13ap11517orf3 | 3418 | 8180 |
| 01cp10622_2472312_c2_2 | 3419 | 8181 |
| 07ae10309_23541676_c3_5 | 3420 | 8182 |
| 07ae11008_10835762_f1_6 | 3421 | 8183 |
| 09cp10502orf8 | 3422 | 8184 |
| 09cp20502_429626_f2_21 | 3423 | 8185 |
| 14gp11820orf24 | 3424 | 8186 |
| 07ae11008orf2 | 3425 | 8187 |
| 07ae11008_4413467_c3_60 | 3426 | 8188 |
| 29zp10241orf17 | 3427 | 8189 |
| 14cp20718_23438768_c2_16 | 3428 | 8190 |
| 07ap11111_23475627_f1_1 | 3429 | 8191 |
| 07ap11111orf6 | 3430 | 8192 |
| 29zp10241orf5 | 3431 | 8193 |
| 14cp20718_23595006_f1_2 | 3432 | 8194 |
| 07ap11111_2382762_f2_5 | 3433 | 8195 |
| 23490686 | 3434 | 8196 |
| 07ap11111orf3 | 3435 | 8197 |
| 07ap61111_24238327_f2_21 | 3436 | 8198 |
| 11cp10113orf6 | 3437 | 8199 |
| 01ae11010_24241562_f1_2 | 3438 | 8200 |
| 07cp10312orf7 | 3439 | 8201 |
| hpy29zp10241orf_0009.aa | 3440 | 8202 |
| 07ap61111_24416693_c2_83 | 3441 | 8203 |
| 11cp10113orf2 | 3442 | 8204 |
| 07ap61111_24740908_c2_88 | 3443 | 8205 |
| 26197187 | 3444 | 8206 |
| 14cp20718_26197187_f2_4 | 3445 | 8207 |
| 29zp10241orf7 | 3446 | 8208 |
| hp6p10233_33397538_f2_9 | 3447 | 8209 |
| 33397538 | 3448 | 8210 |
| hp1p13868orf24 | 3449 | 8211 |
| 07ap11111_4554637_f1_2 | 3450 | 8212 |
| 07ap11111orf12 | 3451 | 8213 |
| 07ap61111_4805338_c1_57 | 3452 | 8214 |
| 07ap61111_4805338_c2_76 | 3453 | 8215 |
| hpyhp3e10349orf_0007.aa | 3454 | 8216 |
| 07ap61111_4976512_c2_94 | 3455 | 8217 |
| 06ap11206_6917518_f3_5 | 3456 | 8218 |
| 07cp10312orf4 | 3457 | 8219 |
| hpyhp3p10180orf_0001.aa | 3458 | 8220 |
| 07ap61111_9938162_c1_57 | 3459 | 8221 |
| hpyhp3e10349orf_0009.aa | 3460 | 8222 |
| 07ce11221_26204752_f1_1 | 3461 | 8223 |
| 07ce11221orf1 | 3462 | 8224 |
| hpy07ce11221orf_0001.aa | 3463 | 8225 |
| 14ee41924_11715_f3_53 | 3464 | 8226 |
| hpy07ap11015orf_0003.aa | 3465 | 8227 |
| 14ee41924_12207031_f1_1 | 3466 | 8228 |
| 07ee11402_19581265_f3_36 | 3467 | 8229 |
| hp5p15861_22697062_f3_9 | 3468 | 8230 |
| 02cp20821orf5 | 3469 | 8231 |
| 14ee41924_24334507_f2_24 | 3470 | 8232 |
| 02ep30607orf11 | 3471 | 8233 |
| 14ee41924_24351577_c2_86 | 3472 | 8234 |
| 02ep30607orf13 | 3473 | 8235 |
| 05ae10307orf2 | 3474 | 8236 |
| 14ee41924_33477177_f3_52 | 3475 | 8237 |
| 14ee41924_665937_f3_46 | 3476 | 8238 |
| 02ep30607orf2 | 3477 | 8239 |
| 02ae31010_10196928_f2_28 | 3478 | 8240 |
| 01ce11618orf2 | 3479 | 8241 |
| 02ae31010_1064125_f1_11 | 3480 | 8242 |
| hpy07ce10203orf_0005.aa | 3481 | 8243 |
| 02ae31010_4396902_c2_58 | 3482 | 8244 |
| 07ee50709_1304651_c1_143 | 3483 | 8245 |
| 07ce10203orf23 | 3484 | 8246 |
| 07ce10203orf18 | 3485 | 8247 |
| 02ae31010_1386712_f1_9 | 3486 | 8248 |
| 02ce11022orf1 | 3487 | 8249 |
| hpy02ce11022orf_0001.aa | 3488 | 8250 |
| 02ae31010_10835802_f2_18 | 3489 | 8251 |
| 07ee50709_14632187_f3_77 | 3490 | 8252 |
| hp2e10911_16308216_c1_72 | 3491 | 8253 |
| hpy01cp11108orf_0003.aa | 3492 | 8254 |
| 07ee50709_16308216_f1_20 | 3493 | 8255 |
| 02ae31010_20991293_c2_73 | 3494 | 8256 |
| 07ce10203orf7 | 3495 | 8257 |
| 02ae31010_22539712_f2_20 | 3496 | 8258 |
| hp5p15641_23534818_f3_11 | 3497 | 8259 |
| hp5p15641orf7 | 3498 | 8260 |
| hp5p15641_24804536_f1_4 | 3499 | 8261 |
| 02ae31010_24898377_f3_37 | 3500 | 8262 |
| hp1p13947orf5 | 3501 | 8263 |
| 02ae31010_250312_f2_24 | 3502 | 8264 |
| hpy07ce10203orf_0004.aa | 3503 | 8265 |
| 05ap10914orf1 | 3504 | 8266 |
| hp5p15641_26367041_c2_30 | 3505 | 8267 |
| hpy07ce10203orf_0002.aa | 3506 | 8268 |
| 07ce10203orf17 | 3507 | 8269 |
| hpy07ce10203orf_0001.aa | 3508 | 8270 |
| 02ae31010_2117087_f3_34 | 3509 | 8271 |
| 02ae31010_26438968_f1_12 | 3510 | 8272 |
| 07ce10203orf21 | 3511 | 8273 |
| 07ee50709_26438968_f2_36 | 3512 | 8274 |
| 01ce11618orf14 | 3513 | 8275 |
| 02ae31010_34063887_f1_16 | 3514 | 8276 |
| 02ae31010_4539193_f3_36 | 3515 | 8277 |
| hpyhp1p13947orf_0009.aa | 3516 | 8278 |
| hp5p15641_5210892_f2_9 | 3517 | 8279 |
| hp5p15641orf4 | 3518 | 8280 |
| hp5p15612orf1 | 3519 | 8281 |
| 13ep10824orf1 | 3520 | 8282 |
| hpy13ep10824orf_0001.aa | 3521 | 8283 |
| hp5p15641_6516005_c1_23 | 3522 | 8284 |
| 02ae31010_828342_f3_38 | 3523 | 8285 |
| hp1p13947orf6 | 3524 | 8286 |
| 882827 | 3525 | 8287 |
| 02ae31010_882827_f1_15 | 3526 | 8288 |
| 01ce11618orf20 | 3527 | 8289 |
| 07ge11521_10739577_c3_34 | 3528 | 8290 |
| 07ge11521_10739577_c3_36 | 3529 | 8291 |
| 02cp11404orf5 | 3530 | 8292 |
| 07ge11521_19536086_c3_40 | 3531 | 8293 |
| hpy02cp11404orf_0011.aa | 3532 | 8294 |
| hpy29ap10306orf_0001.aa | 3533 | 8295 |
| 07ge11521_26757752_f2_16 | 3534 | 8296 |
| 02cp11404orf10 | 3535 | 8297 |
| 07ge11521_4296877_c3_35 | 3536 | 8298 |
| 06cp11217_22343753_f1_3 | 3537 | 8299 |
| hp1p13852orf1 | 3538 | 8300 |
| 06cp11217_23454382_f1_4 | 3539 | 8301 |
| hp1p13852orf2 | 3540 | 8302 |
| 05ae30220_26054027_c3_176 | 3541 | 8303 |
| 06cp11217_3907012_f3_12 | 3542 | 8304 |
| hp1p13852orf11 | 3543 | 8305 |
| 06cp11217_4820443_c2_15 | 3544 | 8306 |
| hp1p13852orf13 | 3545 | 8307 |
| hpyhp4p13446orf_0005.aa | 3546 | 8308 |
| hpyhp4p13446orf_0004.aa | 3547 | 8309 |
| hp6p10904_24218817_f2_7 | 3548 | 8310 |
| hp6p10904_3386011_c3_23 | 3549 | 8311 |
| hp4p13446orf1 | 3550 | 8312 |
| 01ep30520orf1 | 3551 | 8313 |
| 09ce52017_22378887_f1_1 | 3552 | 8314 |
| 01ep30520orf9 | 3553 | 8315 |
| 09ce52017_24238312_f2_9 | 3554 | 8316 |
| hpy06ep11115orf_0001.aa | 3555 | 8317 |
| 09ce52017_25400027_f1_7 | 3556 | 8318 |
| 06ep11115orf2 | 3557 | 8319 |
| 09ce52017_5126933_f3_19 | 3558 | 8320 |
| 09cp10224_10735665_c2_48 | 3559 | 8321 |
| hp1p11244orf2 | 3560 | 8322 |
| hp6e10735_36515689_c2_1 | 3561 | 8323 |
| 09cp10224_15908376_c2_46 | 3562 | 8324 |
| hp4p13402orf12 | 3563 | 8325 |
| 09cp10224_24027187_f2_19 | 3564 | 8326 |
| 09cp10224_24506262_f2_13 | 3565 | 8327 |
| 01ce10516orf9 | 3566 | 8328 |
| 09cp10224_32243937_c3_54 | 3567 | 8329 |
| hp4p13402orf5 | 3568 | 8330 |
| hp1p11244orf15 | 3569 | 8331 |
| 09cp10224_5078443_f1_6 | 3570 | 8332 |
| 06cp30603_210042_c3_154 | 3571 | 8333 |
| hpy05cp20518orf_0010.aa | 3572 | 8334 |
| hp6e20339_22459406_c2_50 | 3573 | 8335 |
| 04gp11213orf17 | 3574 | 8336 |
| hpyhp3e10302orf_0005.aa | 3575 | 8337 |
| hpyhp3e10302orf_0006.aa | 3576 | 8338 |
| 06cp30603_22703965_c3_145 | 3577 | 8339 |
| hpy09cp10713orf_0006.aa | 3578 | 8340 |
| 09cp10713_22870317_c3_32 | 3579 | 8341 |
| 09cp10713_23944133_f1_10 | 3580 | 8342 |
| 06cp30603_26750182_c2_116 | 3581 | 8343 |
| hpy05cp20518orf_0017.aa | 3582 | 8344 |
| hp6e20339_30360883_f1_14 | 3583 | 8345 |
| 19556290 | 3584 | 8346 |
| 04gp11213orf60 | 3585 | 8347 |
| 04gp11213orf18 | 3586 | 8348 |
| hp6e20339_3162753_c2_51 | 3587 | 8349 |
| 09cp10713_31798750_c1_26 | 3588 | 8350 |
| 09cp10713orf24 | 3589 | 8351 |
| 06cp30603_31798962_c2_103 | 3590 | 8352 |
| hpy04gp11213orf_0001.aa | 3591 | 8353 |
| hp6e20339_3253901_c2_44 | 3592 | 8354 |
| 04gp11213orf20 | 3593 | 8355 |
| hp6e20339_32625017_c1_39 | 3594 | 8356 |
| 04gp11213orf13 | 3595 | 8357 |
| hpy05cp20518orf_0024.aa | 3596 | 8358 |
| hpy05cp20518orf_0025.aa | 3597 | 8359 |
| 06cp30603_36115877_c2_114 | 3598 | 8360 |
| hp6e20339_36620953_c2_48 | 3599 | 8361 |
| 04gp11213orf7 | 3600 | 8362 |
| 05cp20518orf36 | 3601 | 8363 |
| 06cp30603_3906693_c1_42 | 3602 | 8364 |
| 7031343 | 3603 | 8365 |
| 09cp10713_7031343_c1_28 | 3604 | 8366 |
| 09cp10713_7031343_c1_27 | 3605 | 8367 |
| 09cp10713orf26 | 3606 | 8368 |
| 06cp30603_9804653_c1_87 | 3607 | 8369 |
| hpy05cp20518orf_0023.aa | 3608 | 8370 |
| hp6e52639_21987687_c3_8 | 3609 | 8371 |
| 01cp11009_23469574_c1_2 | 3610 | 8372 |
| hpy05ap11505orf_0006.aa | 3611 | 8373 |
| hpy11ge10309orf_0005.aa | 3612 | 8374 |
| 05ee21703_24804712_c1_9 | 3613 | 8375 |
| 06cp10218_26176689_c1_2 | 3614 | 8376 |
| hpy05ap11505orf_0002.aa | 3615 | 8377 |
| 13ae10610_5079642_c3_38 | 3616 | 8378 |
| 02ee10520orf5 | 3617 | 8379 |
| 06cp30603_24257137_c2_119 | 3618 | 8380 |
| 32431687 | 3619 | 8381 |
| 04ep71403orf13 | 3620 | 8382 |
| hpy04ep71403orf_0008.aa | 3621 | 8383 |
| 12ae10622_34020812_f1_7 | 3622 | 8384 |
| 12ge10321_34166510_f3_16 | 3623 | 8385 |
| hp4p11352orf11 | 3624 | 8386 |
| hpyhp4p11352orf_0012.aa | 3625 | 8387 |
| 14gp11423_34166510_c2_69 | 3626 | 8388 |
| 12ge10321_3964812_f1_2 | 3627 | 8389 |
| hp4p11352orf3 | 3628 | 8390 |
| 12ge10321_4040928_f3_13 | 3629 | 8391 |
| 4040928 | 3630 | 8392 |
| hp4p11352orf8 | 3631 | 8393 |
| hp4p11352orf5 | 3632 | 8394 |
| hp4p11352orf9 | 3633 | 8395 |
| 12ae10622_4821082_f2_21 | 3634 | 8396 |
| 03ae10804_10188535_f2_12 | 3635 | 8397 |
| 06ep10306orf20 | 3636 | 8398 |
| 06ep10306orf17 | 3637 | 8399 |
| hpy06ep10306orf_0017.aa | 3638 | 8400 |
| 03ae10804_14878438_c2_38 | 3639 | 8401 |
| 16251627 | 3640 | 8402 |
| 03ae10804_1990878_c2_35 | 3641 | 8403 |
| 06ep10306orf14 | 3642 | 8404 |
| 03ae10804_21698400_c2_32 | 3643 | 8405 |
| 06ep10306orf11 | 3644 | 8406 |
| 03ae10804_26350302_f1_2 | 3645 | 8407 |
| hp5e11726orf5 | 3646 | 8408 |
| 03ae10804_3182663_f1_3 | 3647 | 8409 |
| hp5e11726orf6 | 3648 | 8410 |
| 06ep10306orf8 | 3649 | 8411 |
| 06ep10306orf9 | 3650 | 8412 |
| 03ae10804_4507717_c1_25 | 3651 | 8413 |
| 06ep10306orf16 | 3652 | 8414 |
| 03ae10804_489413_c3_48 | 3653 | 8415 |
| 06ep10306orf6 | 3654 | 8416 |
| hp1e80523_489413_c3_65 | 3655 | 8417 |
| hp5p15006orf1 | 3656 | 8418 |
| hpyhp5p15006orf_0001.aa | 3657 | 8419 |
| 06ge11904orf2 | 3658 | 8420 |
| hpy06ge11904orf_0001.aa | 3659 | 8421 |
| 06ee11611orf2 | 3660 | 8422 |
| hp3e11188_1379531_f3_13 | 3661 | 8423 |
| hp3p10807orf5 | 3662 | 8424 |
| hp5e12930orf4 | 3663 | 8425 |
| hp5e12930orf6 | 3664 | 8426 |
| hp3e11188_26750012_f3_11 | 3665 | 8427 |
| hp3p10807_26750012_f3_9 | 3666 | 8428 |
| hp3p10807_189075_f2_4 | 3667 | 8429 |
| hp3p10807orf6 | 3668 | 8430 |
| hpyhp3p10807orf_0001.aa | 3669 | 8431 |
| hp3e11188_47327_f2_5 | 3670 | 8432 |
| hp3e11188_47327_f2_9 | 3671 | 8433 |
| 05ge11120orf1 | 3672 | 8434 |
| hp3e11188_910656_f2_10 | 3673 | 8435 |
| 23441078 | 3674 | 8436 |
| hp4e13394_23441078_f1_20 | 3675 | 8437 |
| 01ep10216orf6 | 3676 | 8438 |
| hp4e13394_24735767_f1_22 | 3677 | 8439 |
| 06ep11108orf22 | 3678 | 8440 |
| 09cp10405orf2 | 3679 | 8441 |
| 02ap71220orf1 | 3680 | 8442 |
| hp4e13394_36110762_c2_96 | 3681 | 8443 |
| 09ap11406orf9 | 3682 | 8444 |
| 09ap11406orf3 | 3683 | 8445 |
| hp4e13394_3906592_c2_107 | 3684 | 8446 |
| hp4e13394_648452_c3_118 | 3685 | 8447 |
| 11ce11603orf20 | 3686 | 8448 |
| hp4e13394_953883_c2_99 | 3687 | 8449 |
| 11ce11603orf11 | 3688 | 8450 |
| hpy07ap11622orf_0005.aa | 3689 | 8451 |
| 01ce10303_9767312_f1_1 | 3690 | 8452 |
| hpy04ep10206orf_0004.aa | 3691 | 8453 |
| hp4e53394_15789052_f2_44 | 3692 | 8454 |
| 03ee11215orf28 | 3693 | 8455 |
| 03ee11215orf37 | 3694 | 8456 |
| 03ee11215_16991092_c2_28 | 3695 | 8457 |
| 03ee11215_22265691_c1_22 | 3696 | 8458 |
| 22265691 | 3697 | 8459 |
| 03ee11215orf29 | 3698 | 8460 |
| 24407533 | 3699 | 8461 |
| hp5e13045_24407533_c2_12 | 3700 | 8462 |
| 02ae11611orf5 | 3701 | 8463 |
| hp4e53394_267312_f2_47 | 3702 | 8464 |
| hpy07ap11622orf_0003.aa | 3703 | 8465 |
| 12ge10513orf1 | 3704 | 8466 |
| hpy12ge10513orf_0002.aa | 3705 | 8467 |
| hp4e53394_30203287_f3_58 | 3706 | 8468 |
| 3157067 | 3707 | 8469 |
| 03ee11215_3157067_f2_17 | 3708 | 8470 |
| 03ee11215orf15 | 3709 | 8471 |
| hpy03ee11215orf_0003.aa | 3710 | 8472 |
| hpy03ee11215orf_0002.aa | 3711 | 8473 |
| hp4e53394_32618752_c1_81 | 3712 | 8474 |
| hp1e13852_4469063_f1_4 | 3713 | 8475 |
| 04ep10206orf7 | 3714 | 8476 |
| 03ee11215_5350062_f2_15 | 3715 | 8477 |
| 03ee11215orf13 | 3716 | 8478 |
| hp4e53394_6652327_f1_5 | 3717 | 8479 |
| 03ee11215orf18 | 3718 | 8480 |
| hp5e13045_7055257_c3_15 | 3719 | 8481 |
| 02ae11611orf2 | 3720 | 8482 |
| hp4e53394_8562_f3_65 | 3721 | 8483 |
| 07ap11622orf1 | 3722 | 8484 |
| hp4p13376_13828932_c2_5 | 3723 | 8485 |
| hp4p13376_10020149_c1_4 | 3724 | 8486 |
| hp2e10350orf1 | 3725 | 8487 |
| hp4p13376_26673166_f1_1 | 3726 | 8488 |
| 05ce10208_14485263_f1_7 | 3727 | 8489 |
| 14cp11121orf8 | 3728 | 8490 |
| 14cp11121orf2 | 3729 | 8491 |
| 05ce10208_23671877_f2_10 | 3730 | 8492 |
| hp4p62853_23671877_c1_41 | 3731 | 8493 |
| 06ce10808orf1 | 3732 | 8494 |
| hpy06ce10808orf_0001.aa | 3733 | 8495 |
| hp1p13939_33209817_c2_30 | 3734 | 8496 |
| 12gp11822_36125062_c2_12 | 3735 | 8497 |
| 14ae11614orf6 | 3736 | 8498 |
| 15824052 | 3737 | 8499 |
| 06ep30223_15824052_f2_54 | 3738 | 8500 |
| 14gp12015orf12 | 3739 | 8501 |
| hpy02ce30710orf_0003.aa | 3740 | 8502 |
| 06ep30223_22272187_c2_137 | 3741 | 8503 |
| 02ce30710orf4 | 3742 | 8504 |
| 06ep30223_26757775_f3_93 | 3743 | 8505 |
| hp3e11024orf13 | 3744 | 8506 |
| 02ce30710orf1 | 3745 | 8507 |
| 06ep30223_29736011_c1_114 | 3746 | 8508 |
| hp5p10786orf1 | 3747 | 8509 |
| 06ep30223_30500957_c2_135 | 3748 | 8510 |
| 7225666 | 3749 | 8511 |
| 01ep30520orf16 | 3750 | 8512 |
| 06ep20223_35197255_c2_1 | 3751 | 8513 |
| hp3e11024orf11 | 3752 | 8514 |
| 06ep30223_36125005_f2_66 | 3753 | 8515 |
| hp5e15555_36125005_f3_95 | 3754 | 8516 |
| 06ep30223_35197255_c2_142 | 3755 | 8517 |
| hp5e15555_3910937_c2_141 | 3756 | 8518 |
| 4062813 | 3757 | 8519 |
| hp3e11024orf15 | 3758 | 8520 |
| 06ep30223_4062813_f3_95 | 3759 | 8521 |
| hp3e11024orf6 | 3760 | 8522 |
| 06ep30223_4062813_f1_22 | 3761 | 8523 |
| 05cp21223_4725443_f3_14 | 3762 | 8524 |
| hp1e10506orf5 | 3763 | 8525 |
| 09ap10418orf1 | 3764 | 8526 |
| hpy09ap10418orf_0001.aa | 3765 | 8527 |
| hpy02ce30710orf_0001.aa | 3766 | 8528 |
| 06ep30223_7304760_c1_113 | 3767 | 8529 |
| 01ce21104_13789077_c2_71 | 3768 | 8530 |
| hpy05cp11911orf_0014.aa | 3769 | 8531 |
| hpy02ae11612orf_0009.aa | 3770 | 8532 |
| 02ae11612orf27 | 3771 | 8533 |
| 02ae11612_16664827_c2_85 | 3772 | 8534 |
| 02ae11612_19535962_f1_12 | 3773 | 8535 |
| 05cp11911orf14 | 3774 | 8536 |
| 06ep11917_22692800_c3_26 | 3775 | 8537 |
| hp4e14535orf5 | 3776 | 8538 |
| hpy02ae11612orf_0015.aa | 3777 | 8539 |
| hpy02ae11612orf_0014.aa | 3778 | 8540 |
| 01ce21104_24484438_c1_64 | 3779 | 8541 |
| 01ce11104_24803330_f2_3 | 3780 | 8542 |
| 16ep10117orf1 | 3781 | 8543 |
| 01ce21104_4554713_c2_80 | 3782 | 8544 |
| hpy02ae11612orf_0016.aa | 3783 | 8545 |
| hpy02ae11612orf_0019.aa | 3784 | 8546 |
| hpy02ae11612orf_0020.aa | 3785 | 8547 |
| 01ce21104_4804068_c3_100 | 3786 | 8548 |
| 10664078 | 3787 | 8549 |
| 02ae11612_5114693_f1_16 | 3788 | 8550 |
| 05cp11911orf11 | 3789 | 8551 |
| 02ae11612_6837812_f3_45 | 3790 | 8552 |
| 05cp11911orf9 | 3791 | 8553 |
| hp5p15575_4729717_f1_1 | 3792 | 8554 |
| hpyhp5p15575orf_0004.aa | 3793 | 8555 |
| 09ap10515_16212692_f2_1 | 3794 | 8556 |
| 03ge31106orf2 | 3795 | 8557 |
| 01xe21717orf28 | 3796 | 8558 |
| 05ep10815_23441262_f3_61 | 3797 | 8559 |
| hp6p10509_12618813_c2_16 | 3798 | 8560 |
| hpy16ae10508orf_0006.aa | 3799 | 8561 |
| 14864452 | 3800 | 8562 |
| hp6p10509_25417812_c3_18 | 3801 | 8563 |
| 16ae10508orf10 | 3802 | 8564 |
| hp6p10522_79703_f1_1 | 3803 | 8565 |
| 19541302 | 3804 | 8566 |
| hp6p10590_14742212_f2_13 | 3805 | 8567 |
| 11gp11422orf2 | 3806 | 8568 |
| hpy11gp11422orf_0001.aa | 3807 | 8569 |
| hp6p10590_33336566_f3_21 | 3808 | 8570 |
| 05ae10218orf3 | 3809 | 8571 |
| hpy04ee70114orf_0005.aa | 3810 | 8572 |
| hpy04ee70114orf_0003.aa | 3811 | 8573 |
| hp6p10590_4350342_c3_40 | 3812 | 8574 |
| hp6p10590_4688818_c2_33 | 3813 | 8575 |
| 01gp10401orf2 | 3814 | 8576 |
| 4787562 | 3815 | 8577 |
| hp6p10590_4787562_f1_5 | 3816 | 8578 |
| 11gp11422orf1 | 3817 | 8579 |
| 01ep12002_16457267_c2_17 | 3818 | 8580 |
| 04ep10515orf10 | 3819 | 8581 |
| hpy04ep10515orf_0002.aa | 3820 | 8582 |
| hp6p12129_26172077_c1_20 | 3821 | 8583 |
| hp6p12129_26172077_c2_23 | 3822 | 8584 |
| hpyhp3e10302orf_0018.aa | 3823 | 8585 |
| hp3e10302orf24 | 3824 | 8586 |
| hp6p12129_5320151_f3_13 | 3825 | 8587 |
| 14cp10923orf4 | 3826 | 8588 |
| 14cp10923_10181291_f2_8 | 3827 | 8589 |
| 14cp10923_10427207_f2_7 | 3828 | 8590 |
| 14cp10923orf6 | 3829 | 8591 |
| hp1p13922orf2 | 3830 | 8592 |
| hp1p13922orf7 | 3831 | 8593 |
| hp6p12244_10828331_f3_39 | 3832 | 8594 |
| 24611590 | 3833 | 8595 |
| hp1p13922orf30 | 3834 | 8596 |
| 34089087 | 3835 | 8597 |
| hp1p13922orf22 | 3836 | 8598 |
| hp6p12244_14539063_c2_72 | 3837 | 8599 |
| hp6p12244_14628127_c1_51 | 3838 | 8600 |
| hp6p12244_21688926_c1_63 | 3839 | 8601 |
| 14cp10923_22554712_f1_5 | 3840 | 8602 |
| hp5e15653orf2 | 3841 | 8603 |
| 14cp10923_24023442_f1_2 | 3842 | 8604 |
| 14cp10923orf2 | 3843 | 8605 |
| hpyhp1p13922orf_0015.aa | 3844 | 8606 |
| hp6p12244_242291_c1_50 | 3845 | 8607 |
| 03ap20115orf2 | 3846 | 8608 |
| 03ap20115orf1 | 3847 | 8609 |
| hp1p13922orf6 | 3848 | 8610 |
| hp6p12244_24415938_f1_9 | 3849 | 8611 |
| hpyhp1p13922orf_0009.aa | 3850 | 8612 |
| hp1p13922orf17 | 3851 | 8613 |
| hp6p12244_25429505_f3_40 | 3852 | 8614 |
| 3242952 | 3853 | 8615 |
| 14cp10923_3242952_f1_3 | 3854 | 8616 |
| 14cp10923orf3 | 3855 | 8617 |
| hp1p13922orf4 | 3856 | 8618 |
| hp6p12244_33214692_f2_26 | 3857 | 8619 |
| hp1p13922orf3 | 3858 | 8620 |
| hp1p13922orf8 | 3859 | 8621 |
| 16603381 | 3860 | 8622 |
| hp6p12244_33782812_f2_25 | 3861 | 8623 |
| 24492192 | 3862 | 8624 |
| 14cp10923_4864062_f1_1 | 3863 | 8625 |
| 14cp10923orf1 | 3864 | 8626 |
| 06ae11915orf5 | 3865 | 8627 |
| hp6p12244_21975662_f1_8 | 3866 | 8628 |
| hp6p12244_5282838_f3_39 | 3867 | 8629 |
| 10580417 | 3868 | 8630 |
| hp6p22217_10580417_f3_17 | 3869 | 8631 |
| 13ee12016orf19 | 3870 | 8632 |
| hp6p22217_16459375_c3_31 | 3871 | 8633 |
| 16459375 | 3872 | 8634 |
| 13ee12016orf24 | 3873 | 8635 |
| hp6p22217_21667317_c1_22 | 3874 | 8636 |
| 13ee12016orf33 | 3875 | 8637 |
| 13ae10712_22775033_f1_3 | 3876 | 8638 |
| 13ae10712orf3 | 3877 | 8639 |
| 13ae10712_23626592_f1_5 | 3878 | 8640 |
| 01ae11421orf5 | 3879 | 8641 |
| 13ae10712_23850312_f2_9 | 3880 | 8642 |
| 13ae10712orf7 | 3881 | 8643 |
| 02ge10814orf1 | 3882 | 8644 |
| 11ae12004_6906717_f3_17 | 3883 | 8645 |
| 3958537 | 3884 | 8646 |
| hp7e10429_10720208_c3_42 | 3885 | 8647 |
| 07ge20415orf22 | 3886 | 8648 |
| hpy07ge20415orf_0015.aa | 3887 | 8649 |
| hp7e10429_14495812_c1_27 | 3888 | 8650 |
| hp7e10429_24297188_c3_43 | 3889 | 8651 |
| 07ge20415orf23 | 3890 | 8652 |
| 12ap11614_32711508_f3_6 | 3891 | 8653 |
| 12ap11614orf7 | 3892 | 8654 |
| hp7e10429_34172291_c1_26 | 3893 | 8655 |
| 07ge20415orf21 | 3894 | 8656 |
| 07ge20415orf37 | 3895 | 8657 |
| 07ge20415orf36 | 3896 | 8658 |
| hp7e10429_41018_c1_32 | 3897 | 8659 |
| 07ge20415orf29 | 3898 | 8660 |
| hpy07ge20415orf_0021.aa | 3899 | 8661 |
| hp7e10429_4859652_c2_35 | 3900 | 8662 |
| 07ge20415orf18 | 3901 | 8663 |
| 07ge20415orf13 | 3902 | 8664 |
| hp7e10429_5906337_f1_3 | 3903 | 8665 |
| hp5p15504orf4 | 3904 | 8666 |
| hp5p15504orf1 | 3905 | 8667 |
| hpyhp5p15504orf_0003.aa | 3906 | 8668 |
| hp7e10433_32460937_c3_12 | 3907 | 8669 |
| 14ce61516_17000776_c2_29 | 3908 | 8670 |
| 07ee11905orf3 | 3909 | 8671 |
| 11ae80818_22692925_f3_36 | 3910 | 8672 |
| hpy06cp11722orf_0010.aa | 3911 | 8673 |
| hpy14ep11905orf_0010.aa | 3912 | 8674 |
| 14ce61516_236452_f3_15 | 3913 | 8675 |
| 11ae80818_25957137_c3_65 | 3914 | 8676 |
| 11ae80818_39017_f3_35 | 3915 | 8677 |
| 06cp11722orf13 | 3916 | 8678 |
| 11ae80818_3954717_c3_66 | 3917 | 8679 |
| hpy05ae10808orf_0004.aa | 3918 | 8680 |
| 11ae80818_36532292_c2_54 | 3919 | 8681 |
| 03ap21820orf7 | 3920 | 8682 |
| 11ae80818_5885942_c1_48 | 3921 | 8683 |
| hp7e10434_17070306_f2_12 | 3922 | 8684 |
| hp7e10434_24101635_c1_28 | 3923 | 8685 |
| 06ce10515orf5 | 3924 | 8686 |
| hpy06ce10515orf_0009.aa | 3925 | 8687 |
| hp7e10434_31417090_f3_17 | 3926 | 8688 |
| 01gp11016_24220438_c3_17 | 3927 | 8689 |
| 01gp11016orf18 | 3928 | 8690 |
| hp7e30434_31417090_f1_3 | 3929 | 8691 |
| 12ae21111orf2 | 3930 | 8692 |
| hp6e50324_33386312_c1_10 | 3931 | 8693 |
| hp7e10434_3939018_f1_3 | 3932 | 8694 |
| 06ce10515orf6 | 3933 | 8695 |
| hp7e10434_4181638_f1_4 | 3934 | 8696 |
| 06ce10515orf7 | 3935 | 8697 |
| 06ce10515orf3 | 3936 | 8698 |
| 06ce10515orf1 | 3937 | 8699 |
| hp7e10434_4414092_f3_15 | 3938 | 8700 |
| 12ae21111orf1 | 3939 | 8701 |
| hpy12ae21111orf_0001.aa | 3940 | 8702 |
| hp6e50324_23485714_c1_9 | 3941 | 8703 |
| hp7e30434_4767717_f2_20 | 3942 | 8704 |
| hp5p15249orf1 | 3943 | 8705 |
| hpyhp5p15249orf_0001.aa | 3944 | 8706 |
| hp7e10192_12150293_f2_4 | 3945 | 8707 |
| hp7e10192_12150293_f2_2 | 3946 | 8708 |
| hp7p10191_35830427_f1_1 | 3947 | 8709 |
| hpy01ce11513orf_0013.aa | 3948 | 8710 |
| 01ce11513orf5 | 3949 | 8711 |
| 02ep20506_4334693_f3_11 | 3950 | 8712 |
| hp7p10287_4334693_c3_28 | 3951 | 8713 |
| hpy01ce11513orf_0016.aa | 3952 | 8714 |
| 01ce11513orf7 | 3953 | 8715 |
| hp7p10287_4959717_c1_19 | 3954 | 8716 |
| 02ce41018_30156593_c1_27 | 3955 | 8717 |
| hp3p10349orf13 | 3956 | 8718 |
| hp7e10420_34381937_c1_31 | 3957 | 8719 |
| hpy07gp10508orf_0001.aa | 3958 | 8720 |
| 02ce41018_4884662_c2_31 | 3959 | 8721 |
| hp3p10349orf14 | 3960 | 8722 |
| 07ap20216_2239637_f3_8 | 3961 | 8723 |
| 05ap21216orf2 | 3962 | 8724 |
| 11ap20714_26803187_c3_105 | 3963 | 8725 |
| hp5e15440orf22 | 3964 | 8726 |
| 07ap20216_36126715_f3_9 | 3965 | 8727 |
| 05ap21216orf3 | 3966 | 8728 |
| 11ap20714_3917638_c3_100 | 3967 | 8729 |
| hpyhp5e15440orf_0012.aa | 3968 | 8730 |
| 02cp10615_1070377_c3_62 | 3969 | 8731 |
| 11ze30138_12210325_f1_4 | 3970 | 8732 |
| 09gp10306orf10 | 3971 | 8733 |
| 11ze30138_127340_f1_5 | 3972 | 8734 |
| 09gp10306orf8 | 3973 | 8735 |
| 02cp10615_23447942_c2_56 | 3974 | 8736 |
| 09gp10306orf6 | 3975 | 8737 |
| hpy02cp11721orf_0007.aa | 3976 | 8738 |
| hpy02cp11721orf_0008.aa | 3977 | 8739 |
| 02cp10615_23464686_c3_66 | 3978 | 8740 |
| 11ze30138_24062812_f3_13 | 3979 | 8741 |
| 09gp10306orf3 | 3980 | 8742 |
| 11ze30138_24322792_f2_10 | 3981 | 8743 |
| 09gp10306orf11 | 3982 | 8744 |
| 02cp10615_24322792_c1_48 | 3983 | 8745 |
| 09gp10306orf1 | 3984 | 8746 |
| hpy09gp10306orf_0001.aa | 3985 | 8747 |
| hp3p10304orf1 | 3986 | 8748 |
| 02cp10615_24406693_c3_63 | 3987 | 8749 |
| 11ze30138_24406693_f1_3 | 3988 | 8750 |
| 09gp10306orf4 | 3989 | 8751 |
| 11ze30138_29454382_f3_14 | 3990 | 8752 |
| 02cp10615_29454382_c1_49 | 3991 | 8753 |
| hp1p14013_3006541_f3_10 | 3992 | 8754 |
| hp1p14013orf7 | 3993 | 8755 |
| 01ae22001orf4 | 3994 | 8756 |
| 01ce10218orf1 | 3995 | 8757 |
| hpy01ce10218orf_0001.aa | 3996 | 8758 |
| 02cp10615_781266_c1_44 | 3997 | 8759 |
| hp8e10080_33204166_f2_27 | 3998 | 8760 |
| 09gp10306orf9 | 3999 | 8761 |
| 09gp10306orf5 | 4000 | 8762 |
| 11ze30138_34250066_f3_11 | 4001 | 8763 |
| hpy02cp11721orf_0003.aa | 4002 | 8764 |
| 02cp11721orf19 | 4003 | 8765 |
| hp7e10100_4490967_c3_20 | 4004 | 8766 |
| hp6p10606_4960967_c1_20 | 4005 | 8767 |
| hpy13ep12003orf_0004.aa | 4006 | 8768 |
| 5265957 | 4007 | 8769 |
| hp7e10100_5210967_c1_13 | 4008 | 8770 |
| 02cp11721orf13 | 4009 | 8771 |
| hpy01ee11621orf_0002.aa | 4010 | 8772 |
| hpy01ee11621orf_0001.aa | 4011 | 8773 |
| hp6p10606_5368817_f2_14 | 4012 | 8774 |
| hpy09gp10306orf_0007.aa | 4013 | 8775 |
| hpy09gp10306orf_0006.aa | 4014 | 8776 |
| 02cp10615_6831900_c1_47 | 4015 | 8777 |
| 14570443 | 9613 | 9725 |
| 14645905 | 9614 | 9726 |
| 29557266 | 9615 | 9727 |
| 214812 | 9616 | 9728 |
| 1370202 | 9617 | 9729 |
| 4821082 | 9618 | 9730 |
| 34489543 | 9619 | 9731 |
| 21618785 | 9620 | 9732 |
| 22667967 | 9621 | 9733 |
| C. SECRETED PROTEINS | | |
| C.1 Periplasmic proteins | | |
| 03ae10804orf1 | 4016 | 8778 |
| 03ae10804_23473912_f1_1 | 4017 | 8779 |
| hp1e80523_24415942_f3_27 | 4018 | 8780 |
| C.2 Proteins involved in secretion and | | |
| adhesion | | |
| 12ap10324_3906712_f3_5 | 4019 | 8781 |
| 3906712 | 4020 | 8782 |
| 12ap10324orf3 | 4021 | 8783 |
| 07gp31516orf3 | 4022 | 8784 |
| 12ap10324orf6 | 4023 | 8785 |
| 12ap10324orf5 | 4024 | 8786 |
| 12ap10324_4805318_f2_3 | 4025 | 8787 |
| 12ap10324_4805318_f2_6 | 4026 | 8788 |
| 4805318 | 9622 | 9734 |
| C.3 Chaperones | | |
| 50253 | 4027 | 8789 |
| hp5e15211_50253_f2_13 | 4028 | 8790 |
| hp5e15211_50253_f2_16 | 4029 | 8791 |
| hp5e15211orf10 | 4030 | 8792 |
| C.4 Other secreted proteins | | |
| 09cp61003_23593955_c1_79 | 4031 | 8793 |
| hp6p80503_17032125_f1_1 | 4032 | 8794 |
| 01ce61016_23593955_c3_140 | 4033 | 8795 |
| 01ce61016_23609580_c3_139 | 4034 | 8796 |
| 09cp11003_19532625_c3_17 | 4035 | 8797 |
| 09cp61003_19532625_c1_78 | 4036 | 8798 |
| 14ge10705orf14 | 4037 | 8799 |
| 07cp11213orf1 | 4038 | 8800 |
| hpy07cp11213orf_0001.aa | 4039 | 8801 |
| 11ee10423orf4 | 4040 | 8802 |
| 09cp61003_24335762_c3_111 | 4041 | 8803 |
| hp3p10156orf6 | 4042 | 8804 |
| hp3p10156orf2 | 4043 | 8805 |
| hp7p10290_25548812_f3_14 | 4044 | 8806 |
| hp6e12267_4876718_f2_23 | 4045 | 8807 |
| 12ge10305orf5 | 4046 | 8808 |
| 09ap20802orf13 | 4047 | 8809 |
| hp2p10272_22692325_f3_21 | 4048 | 8810 |
| 02ap11117_23495187_c3_81 | 4049 | 8811 |
| 09ap20802orf14 | 4050 | 8812 |
| hp2p10272_23697200_f3_22 | 4051 | 8813 |
| hp2p10272_26829136_f1_1 | 4052 | 8814 |
| hp2p10272orf1 | 4053 | 8815 |
| 29ep10720_289077_f2_12 | 4054 | 8816 |
| 289077 | 4055 | 8817 |
| 11ge10309orf9 | 4056 | 8818 |
| 02gp20706_1203402_c3_58 | 4057 | 8819 |
| 09ge70821orf2 | 4058 | 8820 |
| 02gp20706_15781452_c2_51 | 4059 | 8821 |
| 02ge10116_15781452_c1_87 | 4060 | 8822 |
| 06ge10115orf17 | 4061 | 8823 |
| 36335436 | 4062 | 8824 |
| 02ge10116_36335436_f3_66 | 4063 | 8825 |
| hp5e15276orf14 | 4064 | 8826 |
| 02gp20706_4892558_f3_19 | 4065 | 8827 |
| 01cp11710orf1 | 4066 | 8828 |
| 02ge41622_20730462_f1_19 | 4067 | 8829 |
| hpy13ee10216orf_0035.aa | 4068 | 8830 |
| 02ge11622_21695936_c1_54 | 4069 | 8831 |
| 13ee10216orf57 | 4070 | 8832 |
| 23594838 | 4071 | 8833 |
| 06ee30709_33851038_c3_30 | 4072 | 8834 |
| 01ae11403orf1 | 4073 | 8835 |
| hpy01ae11403orf_0001.aa | 4074 | 8836 |
| 05ae30220_14570443_c2_94 | 4075 | 8837 |
| 05ae20220orf124 | 4076 | 8838 |
| 01cp20708_10628177_c2_50 | 4077 | 8839 |
| 22447252 | 4078 | 8840 |
| 05ep10815_22447252_c3_110 | 4079 | 8841 |
| 07ge11504orf3 | 4080 | 8842 |
| 30283516 | 4081 | 8843 |
| 05ep10815_30283516_c3_109 | 4082 | 8844 |
| 07ge11504orf2 | 4083 | 8845 |
| hp1p10543orf4 | 4084 | 8846 |
| hpyhp1p10543orf_0004.aa | 4085 | 8847 |
| hp1e10554orf1 | 4086 | 8848 |
| 05ep10815_4195292_c1_84 | 4087 | 8849 |
| 24328910 | 4088 | 8850 |
| hp5e15211_24328910_c3_34 | 4089 | 8851 |
| hp5e15211_24328910_c3_38 | 4090 | 8852 |
| hp5e15211orf21 | 4091 | 8853 |
| hp5e15211_819455_c2_24 | 4092 | 8854 |
| hp5e15211orf23 | 4093 | 8855 |
| 11876471 | 4094 | 8856 |
| 04ep41903_11876461_f1_4 | 4095 | 8857 |
| 09ae11601orf4 | 4096 | 8858 |
| 05ae30220_21619067_f3_56 | 4097 | 8859 |
| 05ae20220orf58 | 4098 | 8860 |
| 05ae30220_21720017_f2_23 | 4099 | 8861 |
| 21720017 | 4100 | 8862 |
| 05ae20220orf24 | 4101 | 8863 |
| 05ae30220_24410643_c1_79 | 4102 | 8864 |
| 24410643 | 4103 | 8865 |
| 05ae20220orf92 | 4104 | 8866 |
| hpy05ae20220orf_0031.aa | 4105 | 8867 |
| hpy05ae20220orf_0030.aa | 4106 | 8868 |
| 05ae30220_24415693_c3_175 | 4107 | 8869 |
| 05ae30220_24882812_c3_103 | 4108 | 8870 |
| 05ae20220orf119 | 4109 | 8871 |
| 05ae30220_25953163_c3_98 | 4110 | 8872 |
| 05ae20220orf95 | 4111 | 8873 |
| 4882318 | 4112 | 8874 |
| 14ep11115orf2 | 4113 | 8875 |
| 14ep11115orf1 | 4114 | 8876 |
| 11ee11408_4882318_f3_24 | 4115 | 8877 |
| 05ae30220_80257_f3_46 | 4116 | 8878 |
| 80257 | 4117 | 8879 |
| 05ae20220orf50 | 4118 | 8880 |
| 02ce10213orf5 | 4119 | 8881 |
| 24276587 | 4120 | 8882 |
| 02ce10213orf20 | 4121 | 8883 |
| 02ce10213orf11 | 4122 | 8884 |
| hp5p15212_24276587_f1_2 | 4123 | 8885 |
| hp5p15212_34064750_f2_9 | 4124 | 8886 |
| 02ce10213orf19 | 4125 | 8887 |
| hpy11ce10908orf_0002.aa | 4126 | 8888 |
| 11ce10908orf1 | 4127 | 8889 |
| hpy11ce10908orf_0001.aa | 4128 | 8890 |
| hp5p15212_6928132_c3_34 | 4129 | 8891 |
| 12ae11404orf9 | 4130 | 8892 |
| 12ae11404orf3 | 4131 | 8893 |
| 12ae11404_22303918_f3_6 | 4132 | 8894 |
| 22303918 | 4133 | 8895 |
| 3166040 | 4134 | 8896 |
| 09ae10512_3166040_c1_40 | 4135 | 8897 |
| hp3e11060orf9 | 4136 | 8898 |
| 32462543 | 4137 | 8899 |
| 01ae12001_32462543_c2_43 | 4138 | 8900 |
| 07gp11807orf32 | 4139 | 8901 |
| 34161500 | 4140 | 8902 |
| 719606 | 4141 | 8903 |
| 07gp11807orf54 | 4142 | 8904 |
| 07gp11807orf42 | 4143 | 8905 |
| 06ap10609_3952_c3_55 | 4144 | 8906 |
| 06ap11119_16594193_f1_9 | 4145 | 8907 |
| 11ge10309orf7 | 4146 | 8908 |
| hp6p10723orf7 | 4147 | 8909 |
| 06ap11119_24406401_f3_23 | 4148 | 8910 |
| 11ge10309orf25 | 4149 | 8911 |
| 06ap11119_24406401_f3_23 | 4150 | 8912 |
| 24406401 | 4151 | 8913 |
| 06cp11118orf7 | 4152 | 8914 |
| 06cp11118_212827_c1_17 | 4153 | 8915 |
| 04ce11617orf4 | 4154 | 8916 |
| hpyhp1p11256orf_0002.aa | 4155 | 8917 |
| 05ee10816_14649077_f3_18 | 4156 | 8918 |
| 06ep10615_14649077_f2_30 | 4157 | 8919 |
| 06ep10615_14649077_f3_52 | 4158 | 8920 |
| hp6e10967_23476502_f2_6 | 4159 | 8921 |
| hp2p10625orf5 | 4160 | 8922 |
| hp6e10967_24882750_f2_7 | 4161 | 8923 |
| hp2p10625orf6 | 4162 | 8924 |
| 04ce11617orf10 | 4163 | 8925 |
| 05ee10816_259703_f2_7 | 4164 | 8926 |
| 21687842 | 4165 | 8927 |
| 06ep11202_21687842_c3_35 | 4166 | 8928 |
| hp2e11858orf5 | 4167 | 8929 |
| 06gp10409_4015687_f2_11 | 4168 | 8930 |
| 14cp10119orf1 | 4169 | 8931 |
| 04cp11202_16603425_c2_72 | 4170 | 8932 |
| 04ge11713orf10 | 4171 | 8933 |
| 04cp11202_19797128_f1_5 | 4172 | 8934 |
| 04cp11202_24261588_f2_23 | 4173 | 8935 |
| 06gp71906_24261588_c2_174 | 4174 | 8936 |
| 01ae12021orf5 | 4175 | 8937 |
| 29386577 | 4176 | 8938 |
| 12gp31106_29386577_f2_24 | 4177 | 8939 |
| 13ap11517orf7 | 4178 | 8940 |
| 12gp31106_3024126_f2_25 | 4179 | 8941 |
| 13ap11517orf15 | 4180 | 8942 |
| 12gp31106_24411308_f1_13 | 4181 | 8943 |
| 13ap11517orf10 | 4182 | 8944 |
| 06gp71906_25401078_c2_155 | 4183 | 8945 |
| 06gp71906_3024126_c1_128 | 4184 | 8946 |
| 09cp20502_24001388_c1_31 | 4185 | 8947 |
| 14gp11820orf1 | 4186 | 8948 |
| 14ap10815_16603418_c3_26 | 4187 | 8949 |
| 16603418 | 4188 | 8950 |
| hp3e10349orf24 | 4189 | 8951 |
| 14ap10815_23439055_c2_21 | 4190 | 8952 |
| 23439055 | 4191 | 8953 |
| hp3e10349orf17 | 4192 | 8954 |
| 07ap11111_234693_c3_14 | 4193 | 8955 |
| 07ap11111orf13 | 4194 | 8956 |
| hpyhp2e10911orf_0017.aa | 4195 | 8957 |
| hpyhp2e10911orf_0016.aa | 4196 | 8958 |
| hp2e10911_10213593_c1_73 | 4197 | 8959 |
| hpy01cp11108orf_0002.aa | 4198 | 8960 |
| hpy01cp11108orf_0001.aa | 4199 | 8961 |
| hp2e10911orf24 | 4200 | 8962 |
| hp2e10911orf25 | 4201 | 8963 |
| hp2e10911_35567005_c2_88 | 4202 | 8964 |
| 07ap11213_35401528_c1_21 | 4203 | 8965 |
| 07ee50709_10213593_f3_77 | 4204 | 8966 |
| 02ae31010_16833312_f2_19 | 4205 | 8967 |
| 02ce11022orf2 | 4206 | 8968 |
| 29ge10307orf4 | 4207 | 8969 |
| hp5p15641_24304527_c3_35 | 4208 | 8970 |
| 29ge10307orf3 | 4209 | 8971 |
| hp5p15641_25635452_c3_34 | 4210 | 8972 |
| hp1p13947orf11 | 4211 | 8973 |
| 01ce11618orf1 | 4212 | 8974 |
| hpy01ce11618orf_0001.aa | 4213 | 8975 |
| 02ae31010_34616666_f2_27 | 4214 | 8976 |
| 01ep11504orf5 | 4215 | 8977 |
| hpy01ep11504orf_0005.aa | 4216 | 8978 |
| 02ae31010_35270000_f3_33 | 4217 | 8979 |
| 01ce11618orf3 | 4218 | 8980 |
| 01ce11618orf13 | 4219 | 8981 |
| 02ae31010_36132785_f2_29 | 4220 | 8982 |
| 01cp11108orf6 | 4221 | 8983 |
| hpy01cp11108orf_0006.aa | 4222 | 8984 |
| hpy01cp11108orf_0005.aa | 4223 | 8985 |
| hpy01cp11108orf_0004.aa | 4224 | 8986 |
| 11cp12006orf17 | 4225 | 8987 |
| hp2e10911_960952_c2_86 | 4226 | 8988 |
| 291700 | 4227 | 8989 |
| hp2e10911_13871077_c3_97 | 4228 | 8990 |
| hp2e10911_4882027_c2_87 | 4229 | 8991 |
| hpy11cp12006orf_0001.aa | 4230 | 8992 |
| 07ee50709_960952_f2_47 | 4231 | 8993 |
| 35336707 | 4232 | 8994 |
| 02ce10216orf2 | 4233 | 8995 |
| 02ce10216orf1 | 4234 | 8996 |
| 09ce10413_35336707_f2_9 | 4235 | 8997 |
| 09ce10413_414011_f1_3 | 4236 | 8998 |
| 02ce10216orf6 | 4237 | 8999 |
| 02ce10216orf7 | 4238 | 9000 |
| 02ce10216orf4 | 4239 | 9001 |
| hpy02ce10216orf_0007.aa | 4240 | 9002 |
| 09ce10413_5865665_f1_4 | 4241 | 9003 |
| 09ce52017_29324062_c1_21 | 4242 | 9004 |
| 01ep30520orf24 | 4243 | 9005 |
| 03ge10505orf6 | 4244 | 9006 |
| hpy11cp71403orf_0002.aa | 4245 | 9007 |
| 09ze10333_1457137_f3_11 | 4246 | 9008 |
| 06cp30603_10744075_c3_136 | 4247 | 9009 |
| hpy04gp11213orf_0010.aa | 4248 | 9010 |
| hp6e20339_1190660_c2_46 | 4249 | 9011 |
| 04gp11213orf22 | 4250 | 9012 |
| 06cp30603_21492187_f2_41 | 4251 | 9013 |
| hp6e20339_21492187_c1_40 | 4252 | 9014 |
| 04gp11213orf14 | 4253 | 9015 |
| 23912707 | 4254 | 9016 |
| 09cp10713_23912707_c1_27 | 4255 | 9017 |
| 09cp10713_23912707_c1_26 | 4256 | 9018 |
| 09cp10713orf25 | 4257 | 9019 |
| 06cp30603_2772578_c1_46 | 4258 | 9020 |
| 2843912 | 4259 | 9021 |
| 05cp20518orf41 | 4260 | 9022 |
| 06cp30603_2772578_c2_117 | 4261 | 9023 |
| hp6e20339_34024187_c1_37 | 4262 | 9024 |
| 04gp11213orf11 | 4263 | 9025 |
| hp6e20339_24317062_c3_57 | 4264 | 9026 |
| 04gp11213orf5 | 4265 | 9027 |
| 06cp30603_34024187_f1_20 | 4266 | 9028 |
| 09cp10713_34024187_f1_31 | 4267 | 9029 |
| 06cp30603_4689068_c3_79 | 4268 | 9030 |
| 06cp30603orf15 | 4269 | 9031 |
| 12ge10321_24308513_f3_20 | 4270 | 9032 |
| 06gp11920orf11 | 4271 | 9033 |
| 06gp11920orf7 | 4272 | 9034 |
| 06gp11920orf6 | 4273 | 9035 |
| 12ae10622_30273255_f1_13 | 4274 | 9036 |
| 03ae10804_23485968_c3_47 | 4275 | 9037 |
| 06ep10306orf5 | 4276 | 9038 |
| 03ae10804_16187640_c2_36 | 4277 | 9039 |
| 06ep10306orf15 | 4278 | 9040 |
| hp1e80523_23485968_c2_49 | 4279 | 9041 |
| 01ge11619_22448587_c1_9 | 4280 | 9042 |
| hp3e10342_22448587_c2_15 | 4281 | 9043 |
| hpy06ep11108orf_0007.aa | 4282 | 9044 |
| 06ep11108orf17 | 4283 | 9045 |
| hp4e13394_35957200_f1_21 | 4284 | 9046 |
| hp4e13394_5088562_f3_54 | 4285 | 9047 |
| 7116626 | 4286 | 9048 |
| hp4e13394_5908553_f1_1 | 4287 | 9049 |
| hp4e13394orf2 | 4288 | 9050 |
| 1416312 | 4289 | 9051 |
| 03ee11215_1416312_c3_35 | 4290 | 9052 |
| 03ee11215orf30 | 4291 | 9053 |
| hp4e53394_1416312_c3_119 | 4292 | 9054 |
| 22542803 | 4293 | 9055 |
| 03ee11215_22542803_f1_7 | 4294 | 9056 |
| 03ee11215orf10 | 4295 | 9057 |
| 23631292 | 4296 | 9058 |
| 05ce10208_23631292_f1_6 | 4297 | 9059 |
| 14cp11121orf6 | 4298 | 9060 |
| 05ce10208_26423583_c3_22 | 4299 | 9061 |
| 26423583 | 4300 | 9062 |
| hp1p13939orf9 | 4301 | 9063 |
| hpyhp1p13939orf_0004.aa | 4302 | 9064 |
| 05ce10208_4707035_c2_17 | 4303 | 9065 |
| hpy05ee10411orf_0002.aa | 4304 | 9066 |
| 06ep30223_176437_c2_134 | 4305 | 9067 |
| 2445812 | 4306 | 9068 |
| 06ep30223_2774062_f1_33 | 4307 | 9069 |
| hp3e11024orf22 | 4308 | 9070 |
| hp2e10229orf4 | 4309 | 9071 |
| 06ep30223_5271902_c1_106 | 4310 | 9072 |
| 02ae11612_1074212_f1_1 | 4311 | 9073 |
| 02ae11612orf1 | 4312 | 9074 |
| 01ce11104_10742963_c2_12 | 4313 | 9075 |
| 16ep10117orf8 | 4314 | 9076 |
| 10742963 | 4315 | 9077 |
| 02ae11612_23598175_f1_2 | 4316 | 9078 |
| 02ae11612orf15 | 4317 | 9079 |
| 02ae11612_24609431_f1_17 | 4318 | 9080 |
| 24609431 | 4319 | 9081 |
| 05cp11911orf12 | 4320 | 9082 |
| 05cp11911orf35 | 4321 | 9083 |
| hpy11ee10118orf_0002.aa | 4322 | 9084 |
| 01ce21104_33203250_c3_87 | 4323 | 9085 |
| 02ae11612_33203250_c1_51 | 4324 | 9086 |
| 02ae11612_35704718_f2_21 | 4325 | 9087 |
| 35704718 | 4326 | 9088 |
| 02ae11612orf4 | 4327 | 9089 |
| hp1e10523orf3 | 4328 | 9090 |
| 01ce11104_36125337_c1_8 | 4329 | 9091 |
| hpy14ce10720orf_0006.aa | 4330 | 9092 |
| hp5p15575_26016387_f2_16 | 4331 | 9093 |
| hp5p15575_6140713_f2_18 | 4332 | 9094 |
| 14ce10720orf12 | 4333 | 9095 |
| 26301059 | 4334 | 9096 |
| hp5p23057_16416459_c2_5 | 4335 | 9097 |
| 09ap10515_36147908_f3_2 | 4336 | 9098 |
| 03ge31106orf1 | 4337 | 9099 |
| hpy02ae11211orf_0008.aa | 4338 | 9100 |
| hpyhp1e13813orf_0001.aa | 4339 | 9101 |
| hp6p12244_4881375_c3_97 | 4340 | 9102 |
| 23564012 | 4341 | 9103 |
| hp6p22217_23564012_f1_5 | 4342 | 9104 |
| 13ee12016orf8 | 4343 | 9105 |
| hp6p22217_272058_f1_2 | 4344 | 9106 |
| 272058 | 4345 | 9107 |
| 13ee12016orf5 | 4346 | 9108 |
| 23958179 | 4347 | 9109 |
| 13ee12016orf15 | 4348 | 9110 |
| hpy13ee12016orf_0014.aa | 4349 | 9111 |
| hp6p22217_2922143_f2_9 | 4350 | 9112 |
| 02gp20814_24230058_f1_2 | 4351 | 9113 |
| 24230058 | 4352 | 9114 |
| 14ee10308orf8 | 4353 | 9115 |
| 06ce20610_29298537_c2_32 | 4354 | 9116 |
| 05gp20111orf4 | 4355 | 9117 |
| 13ae10712_29569208_c2_27 | 4356 | 9118 |
| 13ae10712orf15 | 4357 | 9119 |
| 06ce20610_34647187_c2_33 | 4358 | 9120 |
| hpy05gp20111orf_0004.aa | 4359 | 9121 |
| 06ce20610_3913967_c3_36 | 4360 | 9122 |
| 05gp20111orf6 | 4361 | 9123 |
| hp7e10433_6273452_f1_3 | 4362 | 9124 |
| 01ee211.18orf1 | 4363 | 9125 |
| hp7e10433_36339535_f3_3 | 4364 | 9126 |
| hp7e10433_36339535_f3_3 | 4365 | 9127 |
| 11ae80818_35787667_c2_55 | 4366 | 9128 |
| 03ap21820orf16 | 4367 | 9129 |
| 11ae80818_5953343_c1_46 | 4368 | 9130 |
| 03ap21820orf12 | 4369 | 9131 |
| 11ae80818_783127_c3_63 | 4370 | 9132 |
| 03ap21820orf4 | 4371 | 9133 |
| 14cp11908_783127_c1_72 | 4372 | 9134 |
| 12ap10324orf4 | 4373 | 9135 |
| 12ap10324orf8 | 4374 | 9136 |
| 12ap10324_13178562_f3_6 | 4375 | 9137 |
| 09cp21607_7224187_c2_12 | 4376 | 9138 |
| 07gp31516orf9 | 4377 | 9139 |
| hpy07gp10508orf_0002.aa | 4378 | 9140 |
| hp7e10420_24391078_f1_3 | 4379 | 9141 |
| hp6p10606_15664656_c3_32 | 4380 | 9142 |
| hpy13ep12003orf_0008.aa | 4381 | 9143 |
| hpy13ep12003orf_0007.aa | 4382 | 9144 |
| hp6p10606_19546933_c3_31 | 4383 | 9145 |
| 13ep12003orf21 | 4384 | 9146 |
| hp8e10080_19546933_c2_88 | 4385 | 9147 |
| 23493756 | 4386 | 9148 |
| hp6p10606_23493756_c1_21 | 4387 | 9149 |
| 13ep12003orf20 | 4388 | 9150 |
| 2035936 | 9623 | 9735 |
| 2774062 | 9624 | 9736 |
| 13178562 | 9625 | 9737 |
| D. OTHER CELLULAR PROTEINS | | |
| 24104558 | 4389 | 9151 |
| hp7p10290_12693942_f1_5 | 4390 | 9152 |
| hp3p10156orf12 | 4391 | 9153 |
| 24396937 | 4392 | 9154 |
| hp6p80503_20964382_f2_11 | 4393 | 9155 |
| 01ce11016orf14 | 4394 | 9156 |
| hpyhp3p10156orf_0005.aa | 4395 | 9157 |
| hpyhp3p10156orf_0004.aa | 4396 | 9158 |
| hp3p10156orf3 | 4397 | 9159 |
| hpyhp3p10156orf_0006.aa | 4398 | 9160 |
| hp7p10290_25585941_f3_12 | 4399 | 9161 |
| hp6e12267_4095342_f2_17 | 4400 | 9162 |
| 4095342 | 4401 | 9163 |
| 12ge20305orf30 | 4402 | 9164 |
| 12ge10305orf17 | 4403 | 9165 |
| 12ge10305orf21 | 4404 | 9166 |
| hp6e12267_4875342_c2_49 | 4405 | 9167 |
| 01ge11619_13788141_c2_11 | 4406 | 9168 |
| 11ge10309orf4 | 4407 | 9169 |
| 09ap20802orf22 | 4408 | 9170 |
| 09ap20802orf30 | 4409 | 9171 |
| hp2p10272_21671910_c2_38 | 4410 | 9172 |
| hpy09ap20802orf_0021.aa | 4411 | 9173 |
| hp2p10272_24406280_c1_26 | 4412 | 9174 |
| 09ap20802orf21 | 4413 | 9175 |
| 32704686 | 4414 | 9176 |
| 09ap20802orf5 | 4415 | 9177 |
| hp2p10272_34042518_f1_2 | 4416 | 9178 |
| 09ap20802orf8 | 4417 | 9179 |
| 09ap20802orf1 | 4418 | 9180 |
| hp2p10272_3906568_f3_23 | 4419 | 9181 |
| 29ep10720_24220926_f2_8 | 4420 | 9182 |
| hp4e12063orf1 | 4421 | 9183 |
| 11ge10309orf56 | 4422 | 9184 |
| 12ae10224orf1 | 4423 | 9185 |
| 11ge10309orf66 | 4424 | 9186 |
| 29ep10720_24495312_c1_28 | 4425 | 9187 |
| 06ge10115orf19 | 4426 | 9188 |
| 02gp20706_23866562_c2_53 | 4427 | 9189 |
| 14ae10212orf1 | 4428 | 9190 |
| hpy14ae10212orf_0001.aa | 4429 | 9191 |
| 01cp11710orf33 | 4430 | 9192 |
| 02gp20706_6644630_c3_62 | 4431 | 9193 |
| 02gp20706_4083212_c1_43 | 4432 | 9194 |
| 02ge10116_23866562_c3_146 | 4433 | 9195 |
| 02ge10116_36617176_f1_7 | 4434 | 9196 |
| 02ge20116orf19 | 4435 | 9197 |
| 05ep20322orf4 | 4436 | 9198 |
| 05ep20322orf10 | 4437 | 9199 |
| 05ep20322orf2 | 4438 | 9200 |
| hp6p12311_4111712_c1_8 | 4439 | 9201 |
| 02gp20706_4491093_c1_39 | 4440 | 9202 |
| 4491093 | 4441 | 9203 |
| 06ge10115orf12 | 4442 | 9204 |
| 914087 | 4443 | 9205 |
| 02ge11622_25428152_c1_52 | 4444 | 9206 |
| 13ee10216orf55 | 4445 | 9207 |
| 6136430 | 4446 | 9208 |
| 06ee10709_6136430_c1_11 | 4447 | 9209 |
| 06ee10709orf17 | 4448 | 9210 |
| 06ae11405orf8 | 4449 | 9211 |
| 22687687 | 4450 | 9212 |
| 06ae11405orf10 | 4451 | 9213 |
| 05ep10815_2087568_c3_101 | 4452 | 9214 |
| hpy07cp10312orf_0007.aa | 4453 | 9215 |
| hp3e10185_42813_f3_4 | 4454 | 9216 |
| hp6p30249_42813_c1_6 | 4455 | 9217 |
| 01ae11010_26437877_c2_52 | 4456 | 9218 |
| hpyhp4e14522orf_0003.aa | 4457 | 9219 |
| 01ae11010_5891077_c3_56 | 4458 | 9220 |
| hpyhp4e14522orf_0004.aa | 4459 | 9221 |
| hp4p33322_5891077_c2_45 | 4460 | 9222 |
| 05ep10815_9785327_c1_80 | 4461 | 9223 |
| 01xe21717orf20 | 4462 | 9224 |
| 5312712 | 4463 | 9225 |
| 24329712 | 4464 | 9226 |
| hp5e15211_25411557_c1_22 | 4465 | 9227 |
| hp5e15211orf29 | 4466 | 9228 |
| 29ep10720_24432762_c3_39 | 4467 | 9229 |
| 11ge10309orf39 | 4468 | 9230 |
| 14ce31519orf3 | 4469 | 9231 |
| 15039062 | 4470 | 9232 |
| 11ee11408_15039062_c1_35 | 4471 | 9233 |
| 05gp11901orf20 | 4472 | 9234 |
| 07ap80601_24219012_f2_6 | 4473 | 9235 |
| 24219012 | 4474 | 9236 |
| 07ap80601orf12 | 4475 | 9237 |
| hpy05ae20220orf_0028.aa | 4476 | 9238 |
| 05ae30220_4548792_f2_27 | 4477 | 9239 |
| 4548792 | 4478 | 9240 |
| 05ae20220orf6 | 4479 | 9241 |
| 5078593 | 4480 | 9242 |
| 07ap80601_5078593_f1_4 | 4481 | 9243 |
| 07ap80601orf10 | 4482 | 9244 |
| 05ae30220_54628_c1_116 | 4483 | 9245 |
| hp3p21118_54628_c3_3 | 4484 | 9246 |
| hpy05ae20220orf_0024.aa | 4485 | 9247 |
| hpy02ce10213orf_0023.aa | 4486 | 9248 |
| 02ce10213orf32 | 4487 | 9249 |
| 02ce10213orf22 | 4488 | 9250 |
| hp5p15212_24219500_c1_22 | 4489 | 9251 |
| 35417942 | 4490 | 9252 |
| 12ae11404_24789202_f1_2 | 4491 | 9253 |
| 12ae11404orf8 | 4492 | 9254 |
| 03ce21717orf1 | 4493 | 9255 |
| 06ae11016_30579712_f2_21 | 4494 | 9256 |
| 01ae12001_6490640_c1_34 | 4495 | 9257 |
| 01ae12001_3952_c3_52 | 4496 | 9258 |
| 14726542 | 4497 | 9259 |
| 06ap11119_14726542_f3_21 | 4498 | 9260 |
| hp6p10723orf5 | 4499 | 9261 |
| 06ap11119_23831562_f2_14 | 4500 | 9262 |
| 23831562 | 4501 | 9263 |
| hp6p10723orf20 | 4502 | 9264 |
| 32952 | 4503 | 9265 |
| hp6e10967_32952_c2_20 | 4504 | 9266 |
| hp2p10625orf28 | 4505 | 9267 |
| 6495137 | 4506 | 9268 |
| hp6e10967_657638_f3_9 | 4507 | 9269 |
| hp2p10625orf8 | 4508 | 9270 |
| 02ae21214orf1 | 4509 | 9271 |
| hpy02ae21214orf_0001.aa | 4510 | 9272 |
| hp2e11858orf6 | 4511 | 9273 |
| 06ep11202_26353438_c1_22 | 4512 | 9274 |
| 4569693 | 4513 | 9275 |
| 06ep11202_4569693_c2_28 | 4514 | 9276 |
| 06cp20302orf8 | 4515 | 9277 |
| 06cp20302orf6 | 4516 | 9278 |
| 06cp20302orf7 | 4517 | 9279 |
| 06ep11202_792962_c1_18 | 4518 | 9280 |
| 06cp20302orf2 | 4519 | 9281 |
| 06ep11202_792962_c2_26 | 4520 | 9282 |
| 06gp71906_15115637_f2_59 | 4521 | 9283 |
| hp4p11393orf2 | 4522 | 9284 |
| hpy06ap10209orf_0002.aa | 4523 | 9285 |
| hpy02cp11822orf_0001.aa | 4524 | 9286 |
| 06ap10209orf4 | 4525 | 9287 |
| 06ap10209orf1 | 4526 | 9288 |
| hpy06ap10209orf_0001.aa | 4527 | 9289 |
| 04cp11202_23553177_c1_75 | 4528 | 9290 |
| 04cp11202_23553177_c3_109 | 4529 | 9291 |
| hpy13ee11718orf_0003.aa | 4530 | 9292 |
| hpy13ee11718orf_0002.aa | 4531 | 9293 |
| 1038312 | 4532 | 9294 |
| 13ee11718orf2 | 4533 | 9295 |
| 06gp71906_24348416_f2_61 | 4534 | 9296 |
| 3991067 | 4535 | 9297 |
| 04cp11202_24413202_c1_65 | 4536 | 9298 |
| 04ge11713orf41 | 4537 | 9299 |
| 5111308 | 4538 | 9300 |
| 04cp11202_5350012_c2_85 | 4539 | 9301 |
| 04ge11713orf27 | 4540 | 9302 |
| hpyhp1p13868orf_0003.aa | 4541 | 9303 |
| hpyhp1p13868orf_0004.aa | 4542 | 9304 |
| 07cp10312orf9 | 4543 | 9305 |
| 391313 | 4544 | 9306 |
| 14ee41924_236712_f2_31 | 4545 | 9307 |
| 11ce10917orf9 | 4546 | 9308 |
| 4572168 | 4547 | 9309 |
| hp5p15861_4572168_f3_12 | 4548 | 9310 |
| 02cp20821orf8 | 4549 | 9311 |
| hp2e10911_15680337_c3_105 | 4550 | 9312 |
| hpyhp2e10911orf_0011.aa | 4551 | 9313 |
| hp5p15641orf5 | 4552 | 9314 |
| hp5p15612orf3 | 4553 | 9315 |
| hp5p15641_21563752_f2_10 | 4554 | 9316 |
| hpy01ce11618orf_0017.aa | 4555 | 9317 |
| 01ce11618orf9 | 4556 | 9318 |
| 29ge30321_21673965_f2_7 | 4557 | 9319 |
| 01ce11618orf11 | 4558 | 9320 |
| 01ce11618orf27 | 4559 | 9321 |
| 29ge30321_24336712_f1_5 | 4560 | 9322 |
| hp2e10911_24804577_c3_104 | 4561 | 9323 |
| hpyhp2e10911orf_0012.aa | 4562 | 9324 |
| 05ce10613orf2 | 4563 | 9325 |
| 05ce10613orf1 | 4564 | 9326 |
| hpy05ce10613orf_0002.aa | 4565 | 9327 |
| hp5p15641_30273312_c2_28 | 4566 | 9328 |
| hpy11ae10212orf_0001.aa | 4567 | 9329 |
| hp2e10911_32234750_c1_68 | 4568 | 9330 |
| hp1e10506orf6 | 4569 | 9331 |
| 17787558 | 4570 | 9332 |
| hp5p15641_3907968_f1_3 | 4571 | 9333 |
| hp5p15641orf12 | 4572 | 9334 |
| 05ap10914orf3 | 4573 | 9335 |
| hpy05ap10914orf_0001.aa | 4574 | 9336 |
| hp5p15641_5211687_c2_29 | 4575 | 9337 |
| 06cp11217_4897077_f1_6 | 4576 | 9338 |
| hp1p13852orf4 | 4577 | 9339 |
| hp6p10904_2214676_c1_14 | 4578 | 9340 |
| hp4p13446orf3 | 4579 | 9341 |
| hp6p10904_23704412_f2_5 | 4580 | 9342 |
| hp4p13446orf13 | 4581 | 9343 |
| hp6p10904_7089062_c1_16 | 4582 | 9344 |
| hp4p13446orf5 | 4583 | 9345 |
| hp4p13402orf2 | 4584 | 9346 |
| hpyhp4p13402orf_0004.aa | 4585 | 9347 |
| 1256885 | 4586 | 9348 |
| hp4p13402orf1 | 4587 | 9349 |
| 09cp10224_24317005_c3_53 | 4588 | 9350 |
| 06cp30603_26070252_c3_140 | 4589 | 9351 |
| 23573294 | 4590 | 9352 |
| 06cp30603_33984562_c2_56 | 4591 | 9353 |
| 05cp20518orf50 | 4592 | 9354 |
| hpy04gp11213orf_0021.aa | 4593 | 9355 |
| 24414687 | 4594 | 9356 |
| hp6e20339_4017188_f1_1 | 4595 | 9357 |
| 25925 | 4596 | 9358 |
| 12ae10622orf16 | 4597 | 9359 |
| 12ae10622_4726568_c2_61 | 4598 | 9360 |
| 03ae10804_235286_f3_19 | 4599 | 9361 |
| 09ge11604_4804692_c1_8 | 4600 | 9362 |
| hpy05ap11505orf_0005.aa | 4601 | 9363 |
| hp2p10610_21987687_c2_5 | 4602 | 9364 |
| 01ge11619_23711062_c3_14 | 4603 | 9365 |
| 11ge10309orf12 | 4604 | 9366 |
| 01ge11619_24415880_c2_12 | 4605 | 9367 |
| 11ge10309orf5 | 4606 | 9368 |
| 01ge11619_24417813_c1_8 | 4607 | 9369 |
| 11ge10309orf24 | 4608 | 9370 |
| hpyhp3p10807orf_0005.aa | 4609 | 9371 |
| hp3p10807_29343768_f1_1 | 4610 | 9372 |
| hp3p10807orf4 | 4611 | 9373 |
| hp3p10807_29352212_f2_5 | 4612 | 9374 |
| hp3p10807orf7 | 4613 | 9375 |
| hp3e11188_5082842_f3_12 | 4614 | 9376 |
| 06ee11611orf1 | 4615 | 9377 |
| hp4e13394_26182793_f2_45 | 4616 | 9378 |
| hpy06ep11108orf_0004.aa | 4617 | 9379 |
| hpy03ee11215orf_0001.aa | 4618 | 9380 |
| hpyhp3e10975orf_0005.aa | 4619 | 9381 |
| hp4e53394_2082126_c2_102 | 4620 | 9382 |
| hp3e11168orf35 | 4621 | 9383 |
| 05ce10208_5211002_c2_15 | 4622 | 9384 |
| 14cp11121orf10 | 4623 | 9385 |
| 01ge10203orf14 | 4624 | 9386 |
| 01ge10203orf7 | 4625 | 9387 |
| 01ge10203_35281542_c3_16 | 4626 | 9388 |
| hp4p62853_5914693_c3_52 | 4627 | 9389 |
| 01ge10203_860166_f3_9 | 4628 | 9390 |
| 01ge10203orf6 | 4629 | 9391 |
| hp1e10506orf2 | 4630 | 9392 |
| 06ce11002orf3 | 4631 | 9393 |
| 06ce11002orf8 | 4632 | 9394 |
| 06ce11002_194415_f2_4 | 4633 | 9395 |
| 06ep30223_25402187_c1_112 | 4634 | 9396 |
| 3930468 | 4635 | 9397 |
| hpy02ce11220orf_0003.aa | 4636 | 9398 |
| 02ce11220orf2 | 4637 | 9399 |
| 06ep30223_3930468_c1_110 | 4638 | 9400 |
| 06ep30223_4876077_c3_149 | 4639 | 9401 |
| hp3e11024orf34 | 4640 | 9402 |
| hp6e10491_12712706_f3_12 | 4641 | 9403 |
| hpyhp5e15084orf_0002.aa | 4642 | 9404 |
| 30703183 | 4643 | 9405 |
| hp6p10509_12600691_c3_22 | 4644 | 9406 |
| 16ae10508orf14 | 4645 | 9407 |
| 14642217 | 4646 | 9408 |
| 16ae10508orf13 | 4647 | 9409 |
| hp6p10509_14642217_c2_17 | 4648 | 9410 |
| hp6p10509_14642217_c3_25 | 4649 | 9411 |
| hpy04ee70114orf_0001.aa | 4650 | 9412 |
| 04ee70114orf10 | 4651 | 9413 |
| hp6p10590_23440913_c2_31 | 4652 | 9414 |
| hp6p10847_26194552_f2_1 | 4653 | 9415 |
| 14094816 | 4654 | 9416 |
| 12ap10605_14094816_c1_5 | 4655 | 9417 |
| 29gp10119orf6 | 4656 | 9418 |
| hpyhp3e10302orf_0016.aa | 4657 | 9419 |
| hp3e10302orf26 | 4658 | 9420 |
| hp6p12129_16603417_f3_14 | 4659 | 9421 |
| hp6p12129_12542880_c3_29 | 4660 | 9422 |
| hpy04ep10515orf_0001.aa | 4661 | 9423 |
| hp6p12129_17067265_c3_29 | 4662 | 9424 |
| hp6p12129_214055_f1_2 | 4663 | 9425 |
| hp6p12129_214055_f3_17 | 4664 | 9426 |
| hpy29gp10119orf_0003.aa | 4665 | 9427 |
| 12ap10605_30603402_c1_4 | 4666 | 9428 |
| 30603402 | 4667 | 9429 |
| 29gp10119orf5 | 4668 | 9430 |
| hp6p12244_33492712_c3_88 | 4669 | 9431 |
| hp6p12244_32458287_c3_90 | 4670 | 9432 |
| hp5p15653orf3 | 4671 | 9433 |
| hp5p15653orf1 | 4672 | 9434 |
| hp5p15653orf2 | 4673 | 9435 |
| hpyhp5p15653orf_0001.aa | 4674 | 9436 |
| hp4p12005orf2 | 4675 | 9437 |
| hp6p12244_3948467_c1_52 | 4676 | 9438 |
| hp6p12244_3948467_c3_88 | 4677 | 9439 |
| hp6p22217_23470967_f1_4 | 4678 | 9440 |
| 13ee12016orf7 | 4679 | 9441 |
| 01ae11421orf6 | 4680 | 9442 |
| 01ae11421orf1 | 4681 | 9443 |
| 03ge10505orf1 | 4682 | 9444 |
| hpy03ge10505orf_0001.aa | 4683 | 9445 |
| 13ae10712_24415957_f2_13 | 4684 | 9446 |
| 12ap11614_26054702_c1_8 | 4685 | 9447 |
| hp7e10429_26054702_f3_23 | 4686 | 9448 |
| 12ap11614orf8 | 4687 | 9449 |
| 26054702 | 4688 | 9450 |
| 07ge20415orf6 | 4689 | 9451 |
| 24634750 | 4690 | 9452 |
| 14ce21516_24634750_f2_3 | 4691 | 9453 |
| 14ce21516orf3 | 4692 | 9454 |
| hp7e10192_4412568_f2_5 | 4693 | 9455 |
| 02ce10114orf3 | 4694 | 9456 |
| 16440842 | 4695 | 9457 |
| 02ce10114orf1 | 4696 | 9458 |
| hp7e10192_5917593_f1_2 | 4697 | 9459 |
| 02ep20506_24611325_f2_6 | 4698 | 9460 |
| 01ce11513orf17 | 4699 | 9461 |
| hp7p10287_24611325_c2_24 | 4700 | 9462 |
| 07gp10508orf4 | 4701 | 9463 |
| hpy07gp10508orf_0003.aa | 4702 | 9464 |
| hp3p10349orf16 | 4703 | 9465 |
| 02ce41018_25509687_f3_18 | 4704 | 9466 |
| hp5e15440orf19 | 4705 | 9467 |
| 489057 | 9626 | 9738 |
| 5879160 | 9627 | 9739 |
| 2738378 | 9628 | 9740 |
| 24495312 | 9629 | 9741 |
| 16839562 | 9630 | 9742 |
| 21563752 | 9631 | 9743 |
| 194415 | 9632 | 9744 |
| 24300682 | 9633 | 9745 |
| 12897656 | 9634 | 9746 |
| 36594167 | 9635 | 9747 |
| 4492217 | 9636 | 9748 |
| E. MEMBRANE ASSOCIATED PROTEINS | | |
| 09cp61003_1410927_f1_14 | 4706 | 9468 |
| 09cp61003_5079842_c3_112 | 4707 | 9469 |
| 01ce61016_4960283_c2_142 | 4708 | 9470 |
| 09cp61003_78267_c1_61 | 4709 | 9471 |
| 02ge10116_34584465_c3_160 | 4710 | 9472 |
| 02ge41622_14875000_c1_53 | 4711 | 9473 |
| hpy13ee10216orf_0032.aa | 4712 | 9474 |
| 07gp10513_167167_f3_2 | 4713 | 9475 |
| 27ze10351orf8 | 4714 | 9476 |
| hpy27ze10351orf_0014.aa | 4715 | 9477 |
| 05ae30220_19728437_c1_102 | 4716 | 9478 |
| 05ae30220_24244512_c3_172 | 4717 | 9479 |
| hpy05ae20220orf_0037.aa | 4718 | 9480 |
| 05ae30220_26758552_c2_157 | 4719 | 9481 |
| hpy05ae20220orf_0005.aa | 4720 | 9482 |
| 06ap10609_2738188_f3_30 | 4721 | 9483 |
| 07gp11807orf24 | 4722 | 9484 |
| hpy07gp11807orf_0014.aa | 4723 | 9485 |
| hpy07gp11807orf_0015.aa | 4724 | 9486 |
| 06ap10609_31359837_c3_54 | 4725 | 9487 |
| hpy07gp11807orf_0011.aa | 4726 | 9488 |
| hpy07gp11807orf_0012.aa | 4727 | 9489 |
| 06ap10609_99192_c2_45 | 4728 | 9490 |
| hpyhp6p10723orf_0004.aa | 4729 | 9491 |
| hpyhp6p10723orf_0005.aa | 4730 | 9492 |
| 06ap11119_16495313_c1_36 | 4731 | 9493 |
| 06ep10615_25665908_f1_10 | 4732 | 9494 |
| 06gp71906_24417581_f3_93 | 4733 | 9495 |
| hpy04ge11713orf_0012.aa | 4734 | 9496 |
| hpy11ep12011orf_0002.aa | 4735 | 9497 |
| 06gp71906_32219827_c1_125 | 4736 | 9498 |
| hpy12gp11106orf_0009.aa | 4737 | 9499 |
| 06gp71906_34157561_f3_115 | 4738 | 9590 |
| 09cp10707_23541676_f2_1 | 4739 | 9501 |
| 07ae11008_4472192_f2_19 | 4740 | 9502 |
| 14gp11820orf28 | 4741 | 9503 |
| 07ap61111_23540930_c2_82 | 4742 | 9504 |
| hpyhp1p13868orf_0005.aa | 4743 | 9505 |
| 07ap61111_26678562_f1_14 | 4744 | 9506 |
| hp3e10349orf1 | 4745 | 9507 |
| 07cp10312_5880188_f1_1 | 4746 | 9508 |
| 02ae31010_24040901_c3_103 | 4747 | 9509 |
| 02ae31010_992092_f3_39 | 4748 | 9510 |
| hpyhp1p13947orf_0012.aa | 4749 | 9511 |
| 09cp10224_3209752_c1_40 | 4750 | 9512 |
| hpy01ce10516orf_0004.aa | 4751 | 9513 |
| 06cp30603_3985338_c3_126 | 4752 | 9514 |
| 13ae10610_23634567_f3_24 | 4753 | 9515 |
| hpy12ee10904orf_0002.aa | 4754 | 9516 |
| hpy05gp11901orf_0008.aa | 4755 | 9517 |
| hpy05gp11901orf_0007.aa | 4756 | 9518 |
| 11ee11408_23447952_c2_41 | 4757 | 9519 |
| hp3e11188_4882056_c3_34 | 4758 | 9520 |
| hpyhp3p10807orf_0004.aa | 4759 | 9521 |
| hp3e11188_4882056_c1_29 | 4760 | 9522 |
| 11ap20714_4900017_c2_81 | 4761 | 9523 |
| 05ap21216orf23 | 4762 | 9524 |

[In Table 1, “nt” represents nucleotide Seq. ID number and “aa” represents amino acid Seq. ID number]

Definitions

The terms “purified polypeptide” and “isolated polypeptide” and “a substantially pure preparation of a polypeptide” are used interchangeably herein and, as used herein, mean a polypeptide that has been substantially, and preferably completely, separated from other proteins, lipids, and nucleic acids with which it naturally occurs. Preferably, the polypeptide is also separated from substances, e.g., antibodies or gel matrix, e.g., polyacrylamide, which are used to purify it. Preferably, the polypeptide constitutes at least 10, 20, 50, 70, 80 or 95% dry weight of the purified preparation. Preferably, the preparation contains: sufficient polypeptide to allow protein sequencing; at least 1, 10, or 100 μg of the polypeptide; at least 1, 10, or 100 mg of the polypeptide. Furthermore, the terms “purified polypeptide” and “isolated polypeptide” and “a substantially pure preparation of a polypeptide,” as used herein, refer to both a polypeptide obtained from nature or produced by recombinant DNA techniques as described herein.

For example, an “isolated” or “purified” protein or biologically active portion thereof is substantially free of cellular material or other contaminating proteins from the cell or tissue source from which the H. pylori protein is derived, or substantially free from chemical precursors or other chemicals when chemically synthesized. The language “substantially free of cellular material” includes preparations of H. pylori protein in which the protein is separated from cellular components of the cells from which it is isolated or recombinantly produced. In one embodiment, the language “substantially free of cellular material” includes preparations of H. pylori protein having less than about 30% (by dry weight) of non-H. pylori protein (also referred to herein as a “contaminating protein”), more preferably less than about 20% of non-H. pylori protein, still more preferably less than about 10% of non-H. pylori protein, and most preferably less than about 5% non-H. pylori protein. When the H. pylori protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of H. pylori protein in which the protein is separated from chemical precusors or other chemicals which are involved in the synthesis of the protein. In one embodiment, the language “substantially free of chemical precursors or other chemicals” includes preparations of H. pylori protein having less than about 30% (by dry weight) of chemical precursors or non-H. pylori chemicals, more preferably less than about 20% chemical precursors or non-H. pylori chemicals, still more preferably less than about 10% chemical precursors or non-H. pylori chemicals, and most preferably less than about 5% chemical precursors or non-H. pylori chemicals.

A purified preparation of cells refers to, in the case of plant or animal cells, an in vitro preparation of cells and not an entire intact plant or animal. In the case of cultured cells or microbial cells, it consists of a preparation of at least 10% and more preferably 50% of the subject cells.

A purified or isolated or a substantially pure nucleic acid, e.g., a substantially pure DNA, (are terms used interchangeably herein) is a nucleic acid which is one or both of the following: not immediately contiguous with both of the coding sequences with which it is immediately contiguous (i.e., one at the 5′ end and one at the 3′ end) in the naturally-occurring genome of the organism from which the nucleic acid is derived; or which is substantially free of a nucleic acid with which it occurs in the organism from which the nucleic acid is derived. The term includes, for example, a recombinant DNA which is incorporated into a vector, e.g., into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment) independent of other DNA sequences. Substantially pure DNA also includes a recombinant DNA which is part of a hybrid gene encoding additional H. pylori DNA sequence.

A “contig” as used herein is a nucleic acid representing a continuous stretch of genomic sequence of an organism.

An “open reading frame”, also referred to herein as ORF, is a region of nucleic acid which encodes a polypeptide. This region may represent a portion of a coding sequence or a total sequence and can be determined from a stop to stop codon or from a start to stop codon.

As used herein, a “coding sequence” is a nucleic acid which is transcribed into messenger RNA and/or translated into a polypeptide when placed under the control of appropriate regulatory sequences. The boundaries of the coding sequence are determined by a translation start codon at the five prime terminus and a translation stop code at the three prime terminus. A coding sequence can include but is not limited to messenger RNA, synthetic DNA, and recombinant nucleic acid sequences.

A “complement” of a nucleic acid as used herein referes to an anti-parallel or antisense sequence that participates in Watson-Crick base-pairing with the original sequence.

A “gene product” is a protein or structural RNA which is specifically encoded by a gene.

As used herein, the term “probe” refers to a nucleic acid, peptide or other chemical entity which specifically binds to a molecule of interest. Probes are often associated with or capable of associating with a label. A label is a chemical moiety capable of detection. Typical labels comprise dyes, radioisotopes, luminescent and chemiluminescent moieties, fluorophores, enzymes, precipitating agents, amplification sequences, and the like. Similarly, a nucleic acid, peptide or other chemical entity which specifically binds to a molecule of interest and immobilizes such molecule is referred herein as a “capture ligand”. Capture ligands are typically associated with or capable of associating with a support such as nitro-cellulose, glass, nylon membranes, beads, particles and the like. The specificity of hybridization is dependent on conditions such as the base pair composition of the nucleotides, and the temperature and salt concentration of the reaction. These conditions are readily discernable to one of ordinary skill in the art using routine experimentation.

Homologous refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

Nucleic acids are hybridizable to each other when at least one strand of a nucleic acid can anneal to the other nucleic acid under defined stringency conditions. Stringency of hybridization is determined by: (a) the temperature at which hybridization and/or washing is performed; and (b) the ionic strength and polarity of the hybridization and washing solutions. Hybridization requires that the two nucleic acids contain complementary sequences; depending on the stringency of hybridization, however, mismatches may be tolerated. Typically, hybridization of two sequences at high stingency (such as, for example, in a solution of 0.5×SSC, at 65° C.) requires that the sequences be essentially completely homologous. Conditions of intermediate stringency (such as, for example, 2×SSC at 65° C.) and low stringency (such as, for example 2×SSC at 55° C.), require correspondingly less overall complementarity between the hybridizing sequences. (1×SSC is 0.15 M NaCl, 0.015 M Na citrate). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

The terms peptides, proteins, and polypeptides are used interchangeably herein.

As used herein, the term “surface protein” refers to all surface accessible proteins, e.g. inner and outer membrane proteins, proteins adhering to the cell wall, and secreted proteins.

A polypeptide has H. pylori biological activity if it has one, two and preferably more of the following properties: (1) if when expressed in the course of an H. pylori infection, it can promote, or mediate the attachment of H. pylori to a cell; (2) it has an enzymatic activity, structural or regulatory function characteristic of an H. pylori protein; (3) the gene which encodes it can rescue a lethal mutation in an H. pylori gene; (4) or it is immunogenic in a subject. A polypeptide has biological activity if it is an antagonist, agonist, or super-agonist of a polypeptide having one of the above-listed properties.

A biologically active fragment or analog is one having an in vivo or in vitro activity which is characteristic of the H. pylori polypeptides of the invention contained in the Sequence Listing, or of other naturally occurring H. pylori polypeptides, e.g., one or more of the biological activities described herein. Especially preferred are fragments which exist in vivo, e.g., fragments which arise from post transcriptional processing or which arise from translation of alternatively spliced RNA's. Fragments include those expressed in native or endogenous cells as well as those made in expression systems, e.g., in CHO cells. Because peptides such as H. pylori polypeptides often exhibit a range of physiological properties and because such properties may be attributable to different portions of the molecule, a useful H. pylori fragment or H. pylori analog is one which exhibits a biological activity in any biological assay for H. pylori activity. Most preferably the fragment or analog possesses 10%, preferably 40%, more preferably 60%, 70%, 80% or 90% or greater of the activity of H. pylori, in any in vivo or in vitro assay.

Analogs can differ from naturally occurring H. pylori polypeptides in amino acid sequence or in ways that do not involve sequence, or both. Non-sequence modifications include changes in acetylation, methylation, phosphorylation, carboxylation, or glycosylation. Preferred analogs include H. pylori polypeptides (or biologically active fragments thereof) whose sequences differ from the wild-type sequence by one or more conservative amino acid substitutions or by one or more non-conservative amino acid substitutions, deletions, or insertions which do not substantially diminish the biological activity of the H. pylori polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative substitutions can be made in view of the table below.

TABLE 2
CONSERVATIVE AMINO ACID REPLACEMENTS

| For Amino Acid | Code | Replace with any of |
|---|---|---|
| Alanine | A | D-Ala, Gly, beta-Ala, L-Cys, D-Cys |
| Arginine | R | D-Arg, Lys, D-Lys, homo-Arg, |
| | | D-homo-Arg, Met, Ile, |
| | | D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, |
| | | D-homo-Arg, Met, D- |
| | | Met, Ile, D-Ile, Orn, D-Orn |
| Methionine | M | D-Met, S-Me-Cys, Ile, D-Ile, |
| | | Leu, D-Leu, Val, D-Val |
| Phenylalanine | F | D-Phe, Tyr, D-Thr, L-Dopa, |
| | | His, D-His, Trp, D-Trp, |
| | | Trans-3,4, or 5-phenylproline, cis-3,4, |
| | | or 5-phenylproline |
| Proline | P | D-Pro, L-I-thioazolidine-4-carboxylic |
| | | acid, D-or L-1- |
| | | oxazolidine-4-carboxylic acid |
| Serine | S | D-Ser, Thr, D-Thr, allo-Thr, |
| | | Met, D-Met, Met(O), |
| | | D-Met(O), L-Cys, D-Cys |
| Threonine | T | D-Thr, Ser, D-Ser, allo-Thr, |
| | | Met, D-Met, Met(O), |
| | | D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Other analogs within the invention are those with modifications which increase peptide stability; such analogs may contain, for example, one or more non-peptide bonds (which replace the peptide bonds) in the peptide sequence. Also included are: analogs that include residues other than naturally occurring L-amino acids, e.g., D-amino acids or non-naturally occurring or synthetic amino acids, e.g., β or γ amino acids; and cyclic analogs.

As used herein, the term “fragment”, as applied to an H. pylori analog, will ordinarily be at least about 20 residues, more typically at least about 40 residues, preferably at least about 60 residues in length. Fragments of H. pylori polypeptides can be generated by methods known to those skilled in the art. The ability of a candidate fragment to exhibit a biological activity of H. pylori polypeptide can be assessed by methods known to those skilled in the art as described herein. Also included are H. pylori polypeptides containing residues that are not required for biological activity of the peptide or that result from alternative mRNA splicing or alternative protein processing events.

An “immunogenic component” as used herein is a moiety, such as an H. pylori polypeptide, analog or fragment thereof, that is capable of eliciting a humoral and/or cellular immune response in a host animal alone or in combination with an adjuvant.

An “antigenic component” as used herein is a moiety, such as an H. pylori polypeptide, analog or fragment thereof, that is capable of binding to a specific antibody with sufficiently high affinity to form a detectable antigen-antibody complex.

As used herein, the term “transgene” means a nucleic acid (encoding, e.g., one or more polypeptides), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the cell's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of the selected nucleic acid, all operably linked to the selected nucleic acid, and may include an enhancer sequence.

As used herein, the term “transgenic cell” refers to a cell containing a transgene.

As used herein, a “transgenic animal” is any animal in which one or more, and preferably essentially all, of the cells of the animal includes a transgene. The transgene can be introduced into the cell, directly or indirectly by introduction into a precursor of the cell, by way of deliberate genetic manipulation, such as by process of transformation of competent cells or by microinjection or by infection with a recombinant virus. This molecule may be integrated within a chromosome, or it may be extrachromosomally replicating DNA.

The term “antibody” as used herein is intended to include fragments thereof which are specifically reactive with H. pylori polypeptides.

As used herein, the term “cell-specific promoter” means a DNA sequence that serves as a promoter, i.e., regulates expression of a selected DNA sequence operably linked to the promoter, and which effects expression of the selected DNA sequence in specific cells of a tissue. The term also covers so-called “leaky” promoters, which regulate expression of a selected DNA primarily in one tissue, but cause expression in other tissues as well.

Misexpression, as used herein, refers to a non-wild type pattern of gene expression. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

As used herein, “host cells” and other such terms denoting microorganisms or higher eukaryotic cell lines cultured as unicellular entities refers to cells which can become or have been used as recipients for a recombinant vector or other transfer DNA, and include the progeny of the original cell which has been transfected. It is understood by individuals skilled in the art that the progeny of a single parental cell may not necessarily be completely identical in genomic or total DNA compliment to the original parent, due to accident or deliberate mutation.

As used herein, the term “control sequence” refers to a nucleic acid having a base sequence which is recognized by the host organism to effect the expression of encoded sequences to which they are ligated. The nature of such control sequences differs depending upon the host organism; in prokaryotes, such control sequences generally include a promoter, ribosomal binding site, terminators, and in some cases operators; in eukaryotes, generally such control sequences include promoters, terminators and in some instances, enhancers. The term control sequence is intended to include at a minimum, all components whose presence is necessary for expression, and may also include additional components whose presence is advantageous, for example, leader sequences.

As used herein, the term “operably linked” refers to sequences joined or ligated to function in their intended manner. For example, a control sequence is operably linked to coding sequence by ligation in such a way that expression of the coding sequence is achieved under conditions compatible with the control sequence and host cell.

The metabolism of a substance, as used herein, means any aspect of the, expression, function, action, or regulation of the substance. The metabolism of a substance includes modifications, e.g., covalent or non-covalent modifications of the substance. The metabolism of a substance includes modifications, e.g., covalent or non-covalent modification, the substance induces in other substances. The metabolism of a substance also includes changes in the distribution of the substance. The metabolism of a substance includes changes the substance induces in the distribution of other substances.

A “sample” as used herein refers to a biological sample, such as, for example, tissue or fluid isloated from an individual (including without limitation plasma, serum, cerebrospinal fluid, lymph, tears, saliva and tissue sections) or from in vitro cell culture constituents, as well as samples from the environment.

The practice of the invention will employ, unless otherwise indicated, conventional techniques of chemistry, molecular biology, microbiology, recombinant DNA, and immunology, which are within the skill of the art. Such techniques are explained fully in the literature. See e.g., Sambrook, Fritsch, and Maniatis, Molecular Cloning; Laboratory Manual 2nd ed. (1989); DNA Cloning, Volumes I and II (D. N Glover ed. 1985); Oligonucleotide Synthesis (M. J. Gait ed, 1984); Nucleic Acid Hybridization (B. D. Hames & S. J. Higgins eds. 1984); the series, Methods in Enzymoloqy (Academic Press, Inc.), particularly Vol. 154 and Vol. 155 (Wu and Grossman, eds.) and PCR-A Practical Approach (McPherson, Quirke, and Taylor, eds., 1991).

I. Isolation of Nucleic Acids of H. pylori and Uses Therefor

H. pylori Genomic Sequence

This invention provides nucleotide sequences of the genome of H. pylori which thus comprises a DNA sequence library of H. pylori genomic DNA. The detailed description that follows provides nucleotide sequences of H. pylori, and also describes how the sequences were obtained and how ORFs and protein-coding sequences were identified. Also described are methods of using the disclosed H. pylori sequences in methods including diagnostic and therapeutic applications. Furthermore, the library can be used as a database for identification and comparison of medically important sequences in this and other strains of H. pylori.

To determine the genomic sequence of H. pylori, DNA was isolated from a strain of H. pylori (ATCC # 55679; deposited by Genome Therapeutics Corporation, 100 Beaver Street, Waltham, Mass. 02154) and mechanically sheared by nebulization to a median size of 2 kb. Following size fractionation by gel electrophoresis, the fragments were blunt-ended, ligated to adapter oligonucleotides, and cloned into each of 20 different pMPX vectors (Rice et al., abstracts of Meeting of Genome Mapping and Sequencing, Cold Spring Harbor, N.Y., 5/11-5/15, 1994, p. 225) to construct a series of “shotgun” subclone libraries.

DNA sequencing was achieved using multiplex sequencing procedures essentially as disclosed in Church et al., 1988, Science 240:185; U.S. Pat. Nos. 4,942,124 and 5,149,625). DNA was extracted from pooled cultures and subjected to chemical or enzymatic sequencing. Sequencing reactions were resolved by electrophoresis, and the products were transferred and covalently bound to nylon membranes. Finally, the membranes were sequentially hybridized with a series of labelled oligonucleotides complimentary to “tag” sequences present in the different shotgun cloning vectors. In this manner, a large number of sequences could be obtained from a single set of sequencing reactions. The cloning and sequencing procedures are described in more detail in the Exemplification.

Individual sequence reads obtained in this manner were assembled using the FALCON™ program (Church et al., 1994, Automated DNA Sequencing and Analysis, J. C. Venter, ed., Academic Press) and PHRAP (P. Green, Abstracts of DOE Human Genome Program Contractor-Grantee Workshop V, January 1996, p.157). The average contig length was about 3-4 kb.

A variety of approaches are used to order the contigs so as to obtain a continuous sequence representing the entire H. pylori genome. Synthetic oligonucleotides are designed that are complementary to sequences at the end of each contig. These oligonucleotides may be hybridized to libaries of H. pylori genomic DNA in, for example, lambda phage vectors or plasmid vectors to identify clones that contain sequences corresponding to the junctional regions between individual contigs. Such clones are then used to isolate template DNA and the same oligonucleotides are used as primers in polymerase chain reaction (PCR) to amplify junctional fragments, the nucleotide sequence of which is then determined.

The H. pylori sequences were analyzed for the presence of open reading frames (ORFs) comprising at least 180 nucleotides. As a result of the analysis of ORFs based on stop-to-stop codon reads, it should be understood that these ORFs may not correspond to the ORF of a naturally-occurring H. pylori polypeptide. These ORFs may contain start codons which indicate the initiation of protein synthesis of a naturally-occurring H. pylori polypeptide. Such start codons within the ORFs provided herein can be identified by those of ordinary skill in the relevant art, and the resulting ORF and the encoded H. pylori polypeptide is within the scope of this invention. For example, within the ORFs a codon such as AUG or GUG (encoding methionine or valine) which is part of the initiation signal for protein synthesis can be identified and the ORF modified to correspond to a naturally-occurring H. pylori polypeptide. The predicted coding regions were defined by evaluating the coding potential of such sequences with the program GENEMARK™ (Borodovsky and Mclninch, 1993, Comp. Chem. 17:123).

Other H. pylori Nucleic Acids

The nucleic acids of this invention may be obtained directly from the DNA of the above referenced H. pylori strain by using the polymerase chain reaction (PCR). See “PCR, A Practical Approach” (McPherson, Quirke, and Taylor, eds., IRL Press, Oxford, UK, 1991) for details about the PCR. High fidelity PCR can be used to ensure a faithful DNA copy prior to expression. In addition, the authenticity of amplified products can be checked by conventional sequencing methods. Clones carrying the desired sequences described in this invention may also be obtained by screening the libraries by means of the PCR or by hybridization of synthetic oligonucleotide probes to filter lifts of the library colonies or plaques as known in the art (see, e.g., Sambrook et al., Molecular Cloning, A Laboratory Manual 2nd edition, 1989, Cold Spring Harbor Press, NY).

It is also possible to obtain nucleic acids encoding H. pylori polypeptides from a cDNA library in accordance with protocols herein described. A cDNA encoding an H. pylori polypeptide can be obtained by isolating total mRNA from an appropriate strain. Double stranded cDNAs can then be prepared from the total mRNA. Subsequently, the cDNAs can be inserted into a suitable plasmid or viral (e.g., bacteriophage) vector using any one of a number of known techniques. Genes encoding H. pylori polypeptides can also be cloned using established polymerase chain reaction techniques in accordance with the nucleotide sequence information provided by the invention. The nucleic acids of the invention can be DNA or RNA. Preferred nucleic acids of the invention are contained in the Sequence Listing.

The nucleic acids of the invention can also be chemically synthesized using standard techniques. Various methods of chemically synthesizing polydeoxynucleotides are known, including solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein).

Nucleic acids isolated or synthesized in accordance with features of the present invention are useful, by way of example, without limitation, as probes, primers, capture ligands, antisense genes and for developing expression systems for the synthesis of proteins and peptides corresponding to such sequences. As probes, primers, capture ligands and antisense agents, the nucleic acid normally consists of all or part (approximately twenty or more nucleotides for specificity as well as the ability to form stable hybridization products) of the nucleic acids of the invention contained in the Sequence Listing. These uses are described in further detail below.

Probes

A nucleic acid isolated or synthesized in accordance with the sequence of the invention contained in the Sequence Listing can be used as a probe to specifically detect H. pylori. With the sequence information set forth in the present application, sequences of twenty or more nucleotides are identified which provide the desired inclusivity and exclusivity with respect to H. pylori, and extraneous nucleic acids likely to be encountered during hybridization conditions. More preferably, the sequence will comprise at least twenty to thirty nucleotides to convey stability to the hybridization product formed between the probe and the intended target molecules.

Sequences larger than 1000 nucleotides in length are difficult to synthesize but can be generated by recombinant DNA techniques. Individuals skilled in the art will readily recognize that the nucleic acids, for use as probes, can be provided with a label to facilitate detection of a hybridization product.

Nucleic acid isolated and synthesized in accordance with the sequence of the invention contained in the Sequence Listing can also be useful as probes to detect homologous regions (especially homologous genes) of other Helicobacter species using appropriate stringency hybridization conditions as described herein.

Capture Ligand

For use as a capture ligand, the nucleic acid selected in the manner described above with respect to probes, can be readily associated with a support. The manner in which nucleic acid is associated with supports is well known. Nucleic acid having twenty or more nucleotides in a sequence of the invention contained in the Sequence Listing have utility to separate H. pylori nucleic acid from the nucleic acid of each other and other organisms. Nucleic acid having twenty or more nucleotides in a sequence of the invention contained in the Sequence Listing can also have utility to separate other Helicobacter species from each other and from other organisms. Preferably, the sequence will comprise at least twenty nucleotides to convey stability to the hybridization product formed between the probe and the intended target molecules. Sequences larger than 1000 nucleotides in length are difficult to synthesize but can be generated by recombinant DNA techniques.

Primers

Nucleic acid isolated or synthesized in accordance with the sequences described herein have utility as primers for the amplification of H. pylori nucleic acid. These nucleic acids may also have utility as primers for the amplification of nucleic acids in other Helicobacter species. With respect to polymerase chain reaction (PCR) techniques, nucleic acid sequences of ≧10-15 nucleotides of the invention contained in the Sequence Listing have utility in conjunction with suitable enzymes and reagents to create copies of H. pylori nucleic acid. More preferably, the sequence will comprise twenty or more nucleotides to convey stability to the hybridization product formed between the primer and the intended target molecules. Binding conditions of primers greater than 100 nucleotides are more difficult to control to obtain specificity. High fidelity PCR can be used to ensure a faithful DNA copy prior to expression. In addition, amplified products can be checked by conventional sequencing methods.

The copies can be used in diagnostic assays to detect specific sequences, including genes from H. pylori and/or other Helicobacter species. The copies can also be incorporated into cloning and expression vectors to generate polypeptides corresponding to the nucleic acid synthesized by PCR, as is described in greater detail herein.

Antisense

Nucleic acid or nucleic acid-hybridizing derivatives isolated or synthesized in accordance with the sequences described herein have utility as antisense agents to prevent the expression of H. pylori genes. These sequences also have utility as antisense agents to prevent expression of genes of other Helicobacter species.

In one embodiment, nucleic acid or derivatives corresponding to H. pylori nucleic acids is loaded into a suitable carrier such as a liposome or bacteriophage for introduction into bacterial cells. For example, a nucleic acid having twenty or more nucleotides is capable of binding to bacteria nucleic acid or bacteria messenger RNA. Preferably, the antisense nucleic acid is comprised of 20 or more nucleotides to provide necessary stability of a hybridization product of non-naturally occurring nucleic acid and bacterial nucleic acid and/or bacterial messenger RNA. Nucleic acid having a sequence greater than 1000 nucleotides in length is difficult to synthesize but can be generated by recombinant DNA techniques. Methods for loading antisense nucleic acid in liposomes is known in the art as exemplified by U.S. Pat. No. 4,241,046 issued Dec. 23, 1980 to Papahadjopoulos et al.

II. Expression of H. pylori Nucleic Acids

Nucleic acid isolated or synthesized in accordance with the sequences described herein have utility to generate polypeptides. The nucleic acid of the invention exemplified in the Sequence Listing or fragments of the nucleic acid encoding active portions of H. pylori polypeptides can be cloned into suitable vectors or used to isolate nucleic acid. The isolated nucleic acid is combined with suitable DNA linkers and cloned into a suitable vector.

The function of a specific gene or operon can be ascertained by expression in a bacterial strain under conditions where the activity of the gene product(s) specified by the gene or operon in question can be specifically measured. Alternatively, a gene product may be produced in large quantities in an expressing strain for use as an antigen, an industrial reagent, for structural studies, etc. This expression can be accomplished in a mutant strain which lacks the activity of the gene to be tested, or in a strain that does not produce the same gene product(s). This includes, but is not limited to other Helicobacter strains, or other bacterial strains such as E coli, Norcardia, Corynebacterium, Campylobacter, and Streptomyces species. In some cases the expression host will utilize the natural Helicobacter promoter whereas in others, it will be necessary to drive the gene with a promoter sequence derived from the expressing organism (e.g., an E. coli beta-galactosidase promoter for expression in E coli).

To express a gene product using the natural H. pylori promoter, a procedure such as the following can be used. A restriction fragment containing the gene of interest, together with its associated natural promoter element and regulatory sequences (identified using the DNA sequence data) is cloned into an appropriate recombinant plasmid containing an origin of replication that functions in the host organism and an appropriate selectable marker. This can be accomplished by a number of procedures known to those skilled in the art. It is most preferably done by cutting the plasmid and the fragment to be cloned with the same restriction enzyme to produce compatible ends that can be ligated to join the two pieces together. The recombinant plasmid is introduced into the host organism by, for example, electroporation and cells containing the recombinant plasmid are identified by selection for the marker on the plasmid. Expression of the desired gene product is detected using an assay specific for that gene product.

In the case of a gene that requires a different promoter, the body of the gene (coding sequence) is specifically excised and cloned into an appropriate expression plasmid. This subcloning can be done by several methods, but is most easily accomplished by PCR amplification of a specific fragment and ligation into an expression plasmid after treating the PCR product with a restriction enzyme or exonuclease to create suitable ends for cloning.

A suitable host cell for expression of a gene can be any procaryotic or eucaryotic cell. For example, an H. pylori polypeptide can be expressed in bacterial cells such as E. coli, insect cells (baculovirus), yeast, or mammalian cells such as Chinese hamster ovary cell (CHO). Other suitable host cells are known to those skilled in the art.

Expression in eucaryotic cells such as mammalian, yeast, or insect cells can lead to partial or complete glycosylation and/or formation of relevant inter- or intra-chain disulfide bonds of a recombinant peptide product. Examples of vectors for expression in yeast S. cerivisae include pYepSec1 (Baldari. et al., (1987) Embo J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.). Baculovirus vectors available for expression of proteins in cultured insect cells (SF 9 cells) include the pAc series (Smith et al., (1983) Mol. Cell Biol. 3:2156-2165) and the pVL series (Lucklow, V. A., and Summers, M. D., (1989) Virology 170:31-39). Generally, COS cells (Gluzman, Y., (1981) Cell 23:175-182) are used in conjunction with such vectors as pCDM 8 (Aruffo, A. and Seed, B., (1987) Proc. Natl. Acad. Sci. USA 84:8573-8577) for transient amplification/expression in mammalian cells, while CHO (dhfr⁻ Chinese Hamster Ovary) cells are used with vectors such as pMT2PC (Kaufman et al. (1987), EMBO J. 6:187-195) for stable amplification/expression in mammalian cells. Vector DNA can be introduced into mammalian cells via conventional techniques such as calcium phosphate or calcium chloride co-precipitation, DEAE-dextran-mediated transfection, or electroporation. Suitable methods for transforming host cells can be found in Sambrook et al. (Molecular Cloning: A Laboratory Manual, 2nd Edition, Cold Spring Harbor Laboratory press (1989)), and other laboratory textbooks.

Expression in procaryotes is most often carried out in E. coli with either fusion or non-fusion inducible expression vectors. Fusion vectors usually add a number of NH₂ terminal amino acids to the expressed target gene. These NH₂ terminal amino acids often are referred to as a reporter group. Such reporter groups usually serve two purposes: 1) to increase the solubility of the target recombinant protein; and 2) to aid in the purification of the target recombinant protein by acting as a ligand in affinity purification. Often, in fusion expression vectors, a proteolytic cleavage site is introduced at the junction of the reporter group and the target recombinant protein to enable separation of the target recombinant protein from the reporter group subsequent to purification of the fusion protein. Such enzymes, and their cognate recognition sequences, include Factor Xa, thrombin and enterokinase. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase, maltose E binding protein, or protein A, respectively, to the target recombinant protein. A preferred reporter group is poly(His), which may be fused to the amino or carboxy terminus of the protein and which renders the recombinant fusion protein easily purifiable by metal chelate chromatography.

Inducible non-fusion expression vectors include pTrc (Amann et al., (1988) Gene 69:301-315) and pET11d (Studier et al., Gene Expression Technology: Methods in Enzymology 185, Academic Press, San Diego, Calif. (1990) 60-89). While target gene expression relies on host RNA polymerase transcription from the hybrid trp-lac fusion promoter in pTrc, expression of target genes inserted into pET11d relies on transcription from the T7 gn10-lac 0 fusion promoter mediated by coexpressed viral RNA polymerase (T7 gnl). This viral polymerase is supplied by host strains BL21(DE3) or HMS174(DE3) from a resident λ prophage harboring a T7 gnl under the transcriptional control of the lacUV 5 promoter.

For example, a host cell transfected with a nucleic acid vector directing expression of a nucleotide sequence encoding an H. pylori polypeptide can be cultured under appropriate conditions to allow expression of the polypeptide to occur. The polypeptide may be secreted and isolated from a mixture of cells and medium containing the peptide. Alternatively, the polypeptide may be retained cytoplasmically and the cells harvested, lysed and the protein isolated. A cell culture includes host cells, media and other byproducts. Suitable media for cell culture are well known in the art. Polypeptides of the invention can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such polypeptides. Additionally, in many situations, polypeptides can be produced by chemical cleavage of a native protein (e.g., tryptic digestion) and the cleavage products can then be purified by standard techniques.

In the case of membrane bound proteins, these can be isolated from a host cell by contacting a membrane-associated protein fraction with a detergent forming a solubilized complex, where the membrane-associated protein is no longer entirely embedded in the membrane fraction and is solubilized at least to an extent which allows it to be chromatographically isolated from the membrane fraction. Several different criteria are used for choosing a detergent suitable for solubilizing these complexes. For example, one property considered is the ability of the detergent to solubilize the H. pylori protein within the membrane fraction at minimal denaturation of the membrane-associated protein allowing for the activity or functionality of the membrane-associated protein to return upon reconstitution of the protein. Another property considered when selecting the detergent is the critical micelle concentration (CMC) of the detergent in that the detergent of choice preferably has a high CMC value allowing for ease of removal after reconstitution. A third property considered when selecting a detergent is the hydrophobicity of the detergent. Typically, membrane-associated proteins are very hydrophobic and therefore detergents which are also hydrophobic, e.g., the triton series, would be useful for solubilizing the hydrophobic proteins. Another property important to a detergent can be the capability of the detergent to remove the H. pylori protein with minimal protein-protein interaction facilitating further purification. A fifth property of the detergent which should be considered is the charge of the detergent. For example, if it is desired to use ion exchange resins in the purification process then preferably detergent should be an uncharged detergent. Chromatographic techniques which can be used in the final purification step are known in the art and include hydrophobic interaction, lectin affinity, ion exchange, dye affinity and immunoaffinity.

One strategy to maximize recombinant H. pylori peptide expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy would be to alter the nucleic acid encoding an H. pylori peptide to be inserted into an expression vector so that the individual codons for each amino acid would be those preferentially utilized in highly expressed E. coli proteins (Wada et al., (1992) Nuc. Acids Res. 20:2111-2118). Such alteration of nucleic acids of the invention can be carried out by standard DNA synthesis techniques.

The nucleic acids of the invention can also be chemically synthesized using standard techniques. Various methods of chemically synthesizing polydeoxynucleotides are known, including solid-phase synthesis which, like peptide synthesis, has been fully automated in commercially available DNA synthesizers (See, e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein).

III. H. pylori Polypeptides

This invention encompasses isolated H. pylori polypeptides encoded by the disclosed H. pylori genomic sequences, including the polypeptides of the invention contained in the Sequence Listing. Polypeptides of the invention are preferably at least 5 amino acid residues in length. Using the DNA sequence information provided herein, the amino acid sequences of the polypeptides encompassed by the invention can be deduced using methods well-known in the art. It will be understood that the sequence of an entire nucleic acid encoding an H. pylori polypeptide can be isolated and identified based on an ORF that encodes only a fragment of the cognate protein-coding region. This can be acheived, for example, by using the isolated nucleic acid encoding the ORF, or fragments thereof, to prime a polymerase chain reaction with genomic H. pylori DNA as template; this is followed by sequencing the amplified product.

The polypeptides of the invention can be isolated from wild-type or mutant H. pylori cells or from heterologous organisms or cells (including, but not limited to, bacteria, yeast, insect, plant and mammalian cells) into which an H. pylori nucleic acid has been introduced and expressed. In addition, the polypeptides can be part of recombinant fusion proteins.

H. pylori polypeptides of the invention can be chemically synthesized using commercially automated procedures such as those referenced herein.

H. pylori polypeptides of the invention are also intended to include chimeric proteins and truncated proteins as decribed herein.

Chimeric H. pylori Proteins

H. pylori chimeric polypeptides comprise one or more H. pylori polypeptides fused together. These combined sequences can be made by combining two or more genes, or two or more polypeptide encoding sequences, or at least one gene and at least one polypeptide encoding sequence in tandem, and the subsequent expression of the encoded proteins by conventional molecular biological techniques. The combined nucleotide sequences may be composed of a combination of either full length H. pylori nucleotide sequences or fragments of such sequences, e.g., fragments which contain immunologically relevant portions of the encoded H. pylori protein. These chimeric H. pylori proteins then contain the combined or synergistic vaccine potential of each individual H. pylori protein sequence and can be used in vaccine formulations of the invention.

Truncated Gene Expression and Protein Production

H. pylori proteins encoded by a given nucleotide sequence can also be used in a biologically active truncated form. Such truncation can be produced, for example, by the elimination of either 5′ and/or 3′ regions of the encoding nucleotide sequence. These truncations can affect recombinant expression of the encoded protein and/or subsequent purification of the protein. For example, truncation of a nucleotide sequence encoding a predicted export sequence of a specific protein may alter expression of the protein. Alternatively, C-terminal truncation of an H. pylori polypeptide by elimination of the 3′ end of the nucleic acid coding region may also improve protein expression and subsequent purification and use, as is outlined in Example VIIIb low. Deletion of nucleic acid regions encoding internal H. pylori protein regions can also result in improved protein expression, purification and/or efficacy as a vaccine candidate.

IV. Identification of Nucleic Acids Encoding Vaccine Components and Targets for Agents Effective Against H. pylori

The disclosed H. pylori genome sequence includes segments that direct the synthesis of ribonucleic acids and polypeptides, as well as origins of replication, promoters, other types of regulatory sequences, and intergenic nucleic acids. The invention encompasses nucleic acids encoding immunogenic components of vaccines and targets for agents effective against H. pylori. Identification of said immunogenic components involved in the determination of the function of the disclosed sequences can be achieved using a variety of approaches. Non-limiting examples of these approaches are described briefly below.

Homology to known sequences: Computer-assisted comparison of the disclosed H. pylori sequences with previously reported sequences present in publicly available databases is useful for identifying functional H. pylori nucleic acid and polypeptide sequences. It will be understood that protein-coding sequences, for example, may be compared as a whole, and that a high degree of sequence homology between two proteins (such as, for example, >80-90%) at the amino acid level indicates that the two proteins also possess some degree of functional homology, such as, for example, among enzymes involved in metabolism, DNA synthesis, or cell wall synthesis, and proteins involved in transport, cell division, etc. In addition, many structural features of particular protein classes have been identified and correlate with specific consensus sequences, such as, for example, binding domains for nucleotides, DNA, metal ions, and other small molecules; sites for covalent modifications such as phosphorylation, acylation, and the like; sites of protein:protein interactions, etc. These consensus sequences may be quite short and thus may represent only a fraction of the entire protein-coding sequence. Identification of such a feature in an H. pylori sequence is therefore useful in determining the function of the encoded protein and identifying useful targets of antibacterial drugs.

Of particular relevance to the present invention are structural features that are common to secretory, transmembrane, and surface proteins, including secretion signal peptides and hydrophobic transmembrane domains. H. pylori proteins identified as containing putative signal sequences and/or transmembrane domains are useful as immunogenic components of vaccines.

Identification of essential genes: Nucleic acids that encode proteins essential for growth or viability of H. pylori are preferred drug targets. H. pylori genes can be tested for their biological relevance to the organism by examining the effect of deleting and/or disrupting the genes, i.e., by so-called gene “knockout”, using techniques known to those skilled in the relevant art. In this manner, essential genes may be identified.

Strain-specific sequences: Because of the evolutionary relationship between different H. pylori strains, it is believed that the presently disclosed H. pylori sequences are useful for identifying, and/or discriminating between, previously known and new H. pylori strains. It is believed that other H. pylori strains will exhibit at least 70% sequence homology with the presently disclosed sequence. Systematic and routine analyses of DNA sequences derived from samples containing H. pylori strains, and comparison with the present sequence allows for the identification of sequences that can be used to discriminate between strains, as well as those that are common to all H. pylori strains. In one embodiment, the invention provides nucleic acids, including probes, and peptide and polypeptide sequences that discriminate between different strains of H. pylori. Strain-specific components can also be identified functionally by their ability to elicit or react with antibodies that selectively recognize one or more H. pylori strains.

In another embodiment, the invention provides nucleic acids, including probes, and peptide and polypeptide sequences that are common to all H. pylori strains but are not found in other bacterial species.

Specific Example: Determination Of Candidate Protein Antigens For Antibody And Vaccine Development

The selection of candidate protein antigens for vaccine development can be derived from the nucleic acids encoding H. pylori polypeptides. First, the ORF's can be analyzed for homology to other known exported or membrane proteins and analyzed using the discriminant analysis described by Klein, et al. (Klein, P., Kanehsia, M., and DeLisi, C. (1985) Biochimica et Biophysica Acta 815, 468-476) for predicting exported and membrane proteins.

Homology searches can be performed using the BLAST algorithm contained in the Wisconsin Sequence Analysis Package (Genetics Computer Group, University Research Park, 575 Science Drive, Madison, Wis. 53711) to compare each predicted ORF amino acid sequence with all sequences found in the current GenBank, SWISS-PROT and PIR databases. BLAST searches for local alignments between the ORF and the databank sequences and reports a probability score which indicates the probability of finding this sequence by chance in the database. ORF's with significant homology (e.g. probabilities lower than 1×10⁻⁶ that the homology is only due to random chance) to membrane or exported proteins represent protein antigens for vaccine development. Possible functions can be provided to H. pylori genes based on sequence homology to genes cloned in other organisms.

Discriminant analysis (Klein, et al. supra) can be used to examine the ORF amino acid sequences. This algorithm uses the intrinsic information contained in the ORF amino acid sequence and compares it to information derived from the properties of known membrane and exported proteins. This comparison predicts which proteins will be exported, membrane associated or cytoplasmic. ORF amino acid sequences identified as exported or membrane associated by this algorithm are likely protein antigens for vaccine development.

Surface exposed outer membrane proteins are likely to represent the best antigens to provide a protective immune response against H. pylori. Among the algorithms that can be used to aid in prediction of these outer membrane proteins include the presence of an amphipathic beta-sheet region at their C-terminus. This region which has been detected in a large number of outer membrane proteins in Gram negative bacteria is often characterized by hydrophobic residues (Phe or Tyr) approximately at positions 1, 3, 5, 7 and 9 from the C-terminus (e.g., see FIGS. 10-12, block F). In many of these figures of multiple sequence alignments, an asterisk is used to denote amino acid residues shared by all members of the group, and a dot is used to indicate that all members of the group share homologous amino acid residues at that position. Importantly, these sequences have not been detected at the C-termini of periplasmic proteins, thus allowing preliminary distinction between these classes of proteins based on primary sequence data. This phenomenon has been reported previously by Struyve et al. (J. Mol. Biol. 218:141-148, 1991).

Also illustrated in FIGS. 10-12 and in FIG. 13 are additional amino acid sequence motifs found in many outer membrane proteins of H. pylori. The amino acid sequence alignments in FIGS. 10-13 depict portions of the sequence of twenty-three H. pylori proteins (depicted in the single letter amino acid code) labeled with arbitrary names and their amino acid Sequence ID Numbers and shown N-terminal to C-terminal, left to right. Six distinct blocks (labeled A through F) of similar amino acid residues are found including the distinctive hydrophobic residues (Phe or Tyr; F or Y according to the single letter code for amino acid residues) frequently found at positions near the C-terminus of outer membrane proteins. The presence of several shared motifs clearly establishes the similarity between members of this group of proteins.

In addition, outer membrane proteins isolated from H. pylori frequently share a motif near the mature N-terminus (i.e., after processing to remove the secretion signal) as illustrated in the blocked amino acid residues in FIG. 4. FIG. 14 depicts the N-terminal portion of six H. pylori proteins (designated by arbitrary names and their amino acid Sequence ID Numbers and shown N-terminal to C-terminal, left to right). Varied groups of outer membrane proteins share significant homology across most of their sequences. Examples of such protein families are shown in FIGS. 15-17 (proteins are designated by an arbitrary name and their amino acid Sequence ID Numbers and shown N-terminal to C-terminal, left to right).

One skilled in the art would know that these shared sequence motifs are highly significant and establish a similarity among this group of proteins.

Infrequently it is not possible to distinguish between multiple possible nucleotides at a given position in the nucleic acid sequence. In those cases the ambiguities are denoted by an extended alphabet as follows:

These are the official IUPAC-IUB single-letter base codes

| Code | Base Description | |
|---|---|---|
| G | Guanine | |
| A | Adenine | |
| T | Thymine | |
| C | Cytosine | |
| R | Purine | (A or G) |
| Y | Pyrimidine | (C or T or U) |
| M | Amino | (A or C) |
| K | Ketone | (G or T) |
| S | Strong interaction | (C or G) |
| W | Weak interaction | (A or T) |
| H | Not-G | (A or C or T) |
| B | Not-A | (C or G or T) |
| V | Not-T (not-U) | (A or C or G) |
| D | Not-C | (A or G or T) |
| N | Any | (A or C or G or T) |

The amino acid translations of this invention account for the ambiguity in the nucleic acid sequence by translating the ambiguous codon as the letter “X”. In all cases, the permissible amino acid residues at a position are clear from an examination of the nucleic acid sequence based on the standard genetic code.

V. Production of Fragments and Analogs of H. pyloriNucleic Acids and Polypeptides

Based on the discovery of the H. pylori gene products of the invention provided in the Sequence Lsiting, one skilled in the art can alter the disclosed structure (of H. pylori genes), e.g., by producing fragments or analogs and test the newly produced structures for activity. Examples of techniques known to those skilled in the relevant art which allow the production and testing of fragments and analogs are discussed below. These, or analogous methods can be used to make and screen libraries of polypeptides, e.g., libraries of random peptides or libraries of fragments or analogs of cellular proteins for the ability to bind H. pylori polypeptides. Such screens are useful for the identification of inhibitors of H. pylori.

Generation of Fragments

Fragments of a protein can be produced in several ways, e.g., recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide. Expression of the mutagenized DNA produces polypeptide fragments. Digestion with “end-nibbling” endonucleases can thus generate DNA's which encode an array of fragments. DNA's which encode fragments of a protein can also be generated by random shearing, restriction digestion or a combination of the above-discussed methods.

Fragments can also be chemically synthesized using techniques known in the art such as conventional Merrifield solid phase f-Moc or t-Boc chemistry. For example, peptides of the present invention may be arbitrarily divided into fragments of desired length with no overlap of the fragments, or divided into overlapping fragments of a desired length.

Alteration of Nucleic Acids and Polypeptides: Random Methods

Amino acid sequence variants of a protein can be prepared by random mutagenesis of DNA which encodes a protein or a particular domain or region of a protein. Useful methods include PCR mutagenesis and saturation mutagenesis. A library of random amino acid sequence variants can also be generated by the synthesis of a set of degenerate oligonucleotide sequences. (Methods for screening proteins in a library of variants are elsewhere herein).

(A) PCR Mutagenesis

In PCR mutagenesis, reduced Taq polymerase fidelity is used to introduce random mutations into a cloned fragment of DNA (Leung et al., 1989, Technique 1: 11-15). The DNA region to be mutagenized is amplified using the polymerase chain reaction (PCR) under conditions that reduce the fidelity of DNA synthesis by Taq DNA polymerase, e.g., by using a dGTP/dATP ratio of five and adding Mn²⁺ to the PCR reaction. The pool of amplified DNA fragments are inserted into appropriate cloning vectors to provide random mutant libraries.

(B) Saturation Mutagenesis

Saturation mutagenesis allows for the rapid introduction of a large number of single base substitutions into cloned DNA fragments (Mayers et al., 1985, Science 229:242). This technique includes generation of mutations, e.g., by chemical treatment or irradiation of single-stranded DNA in vitro, and synthesis of a complimentary DNA strand. The mutation frequency can be modulated by modulating the severity of the treatment, and essentially all possible base substitutions can be obtained. Because this procedure does not involve a genetic selection for mutant fragments both neutral substitutions, as well as those that alter function, are obtained. The distribution of point mutations is not biased toward conserved sequence elements.

(C) Degenerate Oligonucleotides

A library of homologs can also be generated from a set of degenerate oligonucleotide sequences. Chemical synthesis of a degenerate sequences can be carried out in an automatic DNA synthesizer, and the synthetic genes then ligated into an appropriate expression vector. The synthesis of degenerate oligonucleotides is known in the art (see for example, Narang, S A (1983) Tetrahedron 39:3; Itakura et al. (1981) Recombinant DNA, Proc 3rd Cleveland Sympos. Macromolecules, ed. AG Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Alteration of Nucleic Acids and Polypeptides: Methods for Directed Mutagenesis

Non-random or directed, mutagenesis techniques can be used to provide specific sequences or mutations in specific regions. These techniques can be used to create variants which include, e.g., deletions, insertions, or substitutions, of residues of the known amino acid sequence of a protein. The sites for mutation can be modified individually or in series, e.g., by (1) substituting first with conserved amino acids and then with more radical choices depending upon results achieved, (2) deleting the target residue, or (3) inserting residues of the same or a different class adjacent to the located site, or combinations of options 1-3.

(A) Alanine Scanning Mutagenesis

Alanine scanning mutagenesis is a useful method for identification of certain residues or regions of the desired protein that are preferred locations or domains for mutagenesis, Cunningham and Wells (Science 244:1081-1085, 1989). In alanine scanning, a residue or group of target residues are identified (e.g., charged residues such as Arg, Asp, His, Lys, and Glu) and replaced by a neutral or negatively charged amino acid (most preferably alanine or polyalanine). Replacement of an amino acid can affect the interaction of the amino acids with the surrounding aqueous environment in or outside the cell. Those domains demonstrating functional sensitivity to the substitutions are then refined by introducing further or other variants at or for the sites of substitution. Thus, while the site for introducing an amino acid sequence variation is predetermined, the nature of the mutation per se need not be predetermined. For example, to optimize the performance of a mutation at a given site, alanine scanning or random mutagenesis may be conducted at the target codon or region and the expressed desired protein subunit variants are screened for the optimal combination of desired activity.

(B) Oligonucleotide-Mediated Mutagenesis

Oligonucleotide-mediated mutagenesis is a useful method for preparing substitution, deletion, and insertion variants of DNA, see, e.g., Adelman et al., (DNA 2:183, 1983). Briefly, the desired DNA is altered by hybridizing an oligonucleotide encoding a mutation to a DNA template, where the template is the single-stranded form of a plasmid or bacteriophage containing the unaltered or native DNA sequence of the desired protein. After hybridization, a DNA polymerase is used to synthesize an entire second complementary strand of the template that will thus incorporate the oligonucleotide primer, and will code for the selected alteration in the desired protein DNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. USA, 75: 5765 [1978]).

(C) Cassette Mutagenesis

Another method for preparing variants, cassette mutagenesis, is based on the technique described by Wells et al. (Gene, 34:315[1985]). The starting material is a plasmid (or other vector) which includes the protein subunit DNA to be mutated. The codon(s) in the protein subunit DNA to be mutated are identified. There must be a unique restriction endonuclease site, on each side of the identified mutation site(s). If no such restriction sites exist, they may be generated using the above-described oligonucleotide-mediated mutagenesis method to introduce them at appropriate locations in the desired protein subunit DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are comparable with the ends of the linearized plasmid, such that it can be directly ligated to the plasmid. This plasmid now contains the mutated desired protein subunit DNA sequence.

(D) Combinatorial Mutagenesis

Combinatorial mutagenesis can also be used to generate mutants (Ladner et al., WO 88/06630). In this method, the amino acid sequences for a group of homologs or other related proteins are aligned, preferably to promote the highest homology possible. All of the amino acids which appear at a given position of the aligned sequences can be selected to create a degenerate set of combinatorial sequences. The variegated library of variants is generated by combinatorial mutagenesis at the nucleic acid level, and is encoded by a variegated gene library. For example, a mixture of synthetic oligonucleotides can be enzymatically ligated into gene sequences such that the degenerate set of potential sequences are expressible as individual peptides, or alternatively, as a set of larger fusion proteins containing the set of degenerate sequences.

Other Modifications of H. pylori Nucleic Acids and Polypeptides

It is possible to modify the structure of an H. pylori polypeptide for such purposes as increasing solubility, enhancing stability (e.g., shelf life ex vivo and resistance to proteolytic degradation in vivo). A modified H. pylori protein or peptide can be produced in which the amino acid sequence has been altered, such as by amino acid substitution, deletion, or addition as described herein.

An H. pylori peptide can also be modified by substitution of cysteine residues preferably with alanine, serine, threonine, leucine or glutamic acid residues to minimize dimerization via disulfide linkages. In addition, amino acid side chains of fragments of the protein of the invention can be chemically modified. Another modification is cyclization of the peptide.

In order to enhance stability and/or reactivity, an H. pylori polypeptide can be modified to incorporate one or more polymorphisms in the amino acid sequence of the protein resulting from any natural allelic variation. Additionally, D-amino acids, non-natural amino acids, or non-amino acid analogs can be substituted or added to produce a modified protein within the scope of this invention. Furthermore, an H. pylori polypeptide can be modified using polyethylene glycol (PEG) according to the method of A. Sehon and co-workers (Wie et al., supra) to produce a protein conjugated with PEG. In addition, PEG can be added during chemical synthesis of the protein. Other modifications of H. pylori proteins include reduction/alkylation (Tarr, Methods of Protein Microcharacterization, J. E. Silver ed., Humana Press, Clifton N.J. 155-194 (1986)); acylation (Tarr, supra); chemical coupling to an appropriate carrier (Mishell and Shiigi, eds, Selected Methods in Cellular Immunology, W H Freeman, San Francisco, Calif. (1980), U.S. Pat. No. 4,939,239; or mild formalin treatment (Marsh, (1971) Int. Arch. of Allergy and Appl. Immunol., 41: 199-215).

To facilitate purification and potentially increase solubility of an H. pylori protein or peptide, it is possible to add an amino acid fusion moiety to the peptide backbone. For example, hexa-histidine can be added to the protein for purification by immobilized metal ion affinity chromatography (Hochuli, E. et al., (1988) Bio/Technology, 6: 1321-1325). In addition, to facilitate isolation of peptides free of irrelevant sequences, specific endoprotease cleavage sites can be introduced between the sequences of the fusion moiety and the peptide.

To potentially aid proper antigen processing of epitopes within an H. pylori polypeptide, canonical protease sensitive sites can be engineered between regions, each comprising at least one epitope via recombinant or synthetic methods. For example, charged amino acid pairs, such as KK or RR, can be introduced between regions within a protein or fragment during recombinant construction thereof. The resulting peptide can be rendered sensitive to cleavage by cathepsin and/or other trypsin-like enzymes which would generate portions of the protein containing one or more epitopes. In addition, such charged amino acid residues can result in an increase in the solubility of the peptide.

Primary Methods for Screening Polypeptides and Analogs

Various techniques are known in the art for screening generated mutant gene products. Techniques for screening large gene libraries often include cloning the gene library into replicable expression vectors, transforming appropriate cells with the resulting library of vectors, and expressing the genes under conditions in which detection of a desired activity, e.g., in this case, binding to H. pylori polypeptide or an interacting protein, facilitates relatively easy isolation of the vector encoding the gene whose product was detected. Each of the techniques described below is amenable to high through-put analysis for screening large numbers of sequences created, e.g., by random mutagenesis techniques.

(A) Two Hybrid Systems

Two hybrid assays such as the system described above (as with the other screening methods described herein), can be used to identify polypeptides, e.g., fragments or analogs of a naturally-occurring H. pylori polypeptide, e.g., of cellular proteins, or of randomly generated polypeptides which bind to an H. pylori protein. (The H. pylori domain is used as the bait protein and the library of variants are expressed as fish fusion proteins.) In an analogous fashion, a two hybrid assay (as with the other screening methods described herein), can be used to find polypeptides which bind a H. pylori polypeptide.

(B) Display Libraries

In one approach to screening assays, the candidate peptides are displayed on the surface of a cell or viral particle, and the ability of particular cells or viral particles to bind an appropriate receptor protein via the displayed product is detected in a “panning assay”. For example, the gene library can be cloned into the gene for a surface membrane protein of a bacterial cell, and the resulting fusion protein detected by panning (Ladner et al., WO 88/06630; Fuchs et al. (1991) Bio/Technology 9:1370-1371; and Goward et al. (1992) TIBS 18:136-140). In a similar fashion, a detectably labeled ligand can be used to score for potentially functional peptide homologs. Fluorescently labeled ligands, e.g., receptors, can be used to detect homologs which retain ligand-binding activity. The use of fluorescently labeled ligands, allows cells to be visually inspected and separated under a fluorescence microscope, or, where the morphology of the cell permits, to be separated by a fluorescence-activated cell sorter.

A gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at concentrations well over 10¹³ phage per milliliter, a large number of phage can be screened at one time. Second, since each infectious phage displays a gene product on its surface, if a particular phage is recovered from an affinity matrix in low yield, the phage can be amplified by another round of infection. The group of almost identical E. coli filamentous phages M13, fd., and fl are most often used in phage display libraries. Either of the phage gIII or gVIII coat proteins can be used to generate fusion proteins without disrupting the ultimate packaging of the viral particle. Foreign epitopes can be expressed at the NH₂-terminal end of pIII and phage bearing such epitopes recovered from a large excess of phage lacking this epitope (Ladner et al. PCT publication WO 90/02909; Garrard et al., PCT publication WO 92/09690; Marks et al. (1992) J. Biol. Chem. 267:16007-16010; Griffiths et al. (1993) EMBO J. 12:725-734; Clackson et al. (1991) Nature 352:624-628; and Barbas et al. (1992) PNAS 89:4457-4461).

A common approach uses the maltose receptor of E. coli (the outer membrane protein, LamB) as a peptide fusion partner (Charbit et al. (1986) EMBO 5, 3029-3037). Oligonucleotides have been inserted into plasmids encoding the LamB gene to produce peptides fused into one of the extracellular loops of the protein. These peptides are available for binding to ligands, e.g., to antibodies, and can elicit an immune response when the cells are administered to animals. Other cell surface proteins, e.g., OmpA (Schorr et al. (1991) Vaccines 91, pp. 387-392), PhoE (Agterberg, et al. (1990) Gene 88, 37-45), and PAL (Fuchs et al. (1991) Bio/Tech 9, 1369-1372), as well as large bacterial surface structures have served as vehicles for peptide display. Peptides can be fused to pilin, a protein which polymerizes to form the pilus-a conduit for interbacterial exchange of genetic information (Thiry et al. (1989) Appl. Environ. Microbiol. 55, 984-993). Because of its role in interacting with other cells, the pilus provides a useful support for the presentation of peptides to the extracellular environment. Another large surface structure used for peptide display is the bacterial motive organ, the flagellum. Fusion of peptides to the subunit protein flagellin offers a dense array of many peptide copies on the host cells (Kuwajima et al. (1988) Bio/Tech. 6, 1080-1083). Surface proteins of other bacterial species have also served as peptide fusion partners. Examples include the Staphylococcus protein A and the outer membrane IgA protease of Neisseria (Hansson et al. (1992) J. Bacteriol. 174, 4239-4245 and Klauser et al. (1990) EMBO J. 9, 1991-1999).

In the filamentous phage systems and the LamB system described above, the physical link between the peptide and its encoding DNA occurs by the containment of the DNA within a particle (cell or phage) that carries the peptide on its surface. Capturing the peptide captures the particle and the DNA within. An alternative scheme uses the DNA-binding protein LacI to form a link between peptide and DNA (Cull et al. (1992) PNAS USA 89:1865-1869). This system uses a plasmid containing the LacI gene with an oligonucleotide cloning site at its 3′-end. Under the controlled induction by arabinose, a LacI-peptide fusion protein is produced. This fusion retains the natural ability of LacI to bind to a short DNA sequence known as LacO operator (LacO). By installing two copies of LacO on the expression plasmid, the LacI-peptide fusion binds tightly to the plasmid that encoded it. Because the plasmids in each cell contain only a single oligonucleotide sequence and each cell expresses only a single peptide sequence, the peptides become specifically and stably associated with the DNA sequence that directed its synthesis. The cells of the library are gently lysed and the peptide-DNA complexes are exposed to a matrix of immobilized receptor to recover the complexes containing active peptides. The associated plasmid DNA is then reintroduced into cells for amplification and DNA sequencing to determine the identity of the peptide ligands. As a demonstration of the practical utility of the method, a large random library of dodecapeptides was made and selected on a monoclonal antibody raised against the opioid peptide dynorphin B. A cohort of peptides was recovered, all related by a consensus sequence corresponding to a six-residue portion of dynorphin B. (Cull et al. (1992) Proc. Natl. Acad. Sci. USA. 89-1869)

This scheme, sometimes referred to as peptides-on-plasmids, differs in two important ways from the phage display methods. First, the peptides are attached to the C-terminus of the fusion protein, resulting in the display of the library members as peptides having free carboxy termini. Both of the filamentous phage coat proteins, pill and pVIII, are anchored to the phage through their C-termini, and the guest peptides are placed into the outward-extending N-terminal domains. In some designs, the phage-displayed peptides are presented right at the amino terminus of the fusion protein. (Cwirla, et al. (1990) Proc. Natl. Acad. Sci. U.S.A. 87, 6378-6382) A second difference is the set of biological biases affecting the population of peptides actually present in the libraries. The LacI fusion molecules are confined to the cytoplasm of the host cells. The phage coat fusions are exposed briefly to the cytoplasm during translation but are rapidly secreted through the inner membrane into the periplasmic compartment, remaining anchored in the membrane by their C-terminal hydrophobic domains, with the N-termini, containing the peptides, protruding into the periplasm while awaiting assembly into phage particles. The peptides in the LacI and phage libraries may differ significantly as a result of their exposure to different proteolytic activities. The phage coat proteins require transport across the inner membrane and signal peptidase processing as a prelude to incorporation into phage. Certain peptides exert a deleterious effect on these processes and are underrepresented in the libraries (Gallop et al. (1994) J. Med. Chem. 37(9):1233-¹²⁵I). These particular biases are not a factor in the LacI display system.

The number of small peptides available in recombinant random libraries is enormous. Libraries of 10⁷-10⁹ independent clones are routinely prepared. Libraries as large as 10¹¹ recombinants have been created, but this size approaches the practical limit for clone libraries. This limitation in library size occurs at the step of transforming the DNA containing randomized segments into the host bacterial cells. To circumvent this limitation, an in vitro system based on the display of nascent peptides in polysome complexes has recently been developed. This display library method has the potential of producing libraries 3-6 orders of magnitude larger than the currently available phage/phagemid or plasmid libraries. Furthermore, the construction of the libraries, expression of the peptides, and screening, is done in an entirely cell-free format.

In one application of this method (Gallop et al. (1994) J. Med. Chem. 37(9):1233-1251), a molecular DNA library encoding 10¹² decapeptides was constructed and the library expressed in an E. coli S30 in vitro coupled transcription/translation system. Conditions were chosen to stall the ribosomes on the mRNA, causing the accumulation of a substantial proportion of the RNA in polysomes and yielding complexes containing nascent peptides still linked to their encoding RNA. The polysomes are sufficiently robust to be affinity purified on immobilized receptors in much the same way as the more conventional recombinant peptide display libraries are screened. RNA from the bound complexes is recovered, converted to cDNA, and amplified by PCR to produce a template for the next round of synthesis and screening. The polysome display method can be coupled to the phage display system. Following several rounds of screening, cDNA from the enriched pool of polysomes was cloned into a phagemid vector. This vector serves as both a peptide expression vector, displaying peptides fused to the coat proteins, and as a DNA sequencing vector for peptide identification. By expressing the polysome-derived peptides on phage, one can either continue the affinity selection procedure in this format or assay the peptides on individual clones for binding activity in a phage ELISA, or for binding specificity in a completion phage ELISA (Barret, et al. (1992) Anal. Biochem 204,357-364). To identify the sequences of the active peptides one sequences the DNA produced by the phagemid host.

Secondary Screening of Polypeptides and Analogs

The high through-put assays described above can be followed by secondary screens in order to identify further biological activities which will, e.g., allow one skilled in the art to differentiate agonists from antagonists. The type of a secondary screen used will depend on the desired activity that needs to be tested. For example, an assay can be developed in which the ability to inhibit an interaction between a protein of interest and its respective ligand can be used to identify antagonists from a group of peptide fragments isolated though one of the primary screens described above.

Therefore, methods for generating fragments and analogs and testing them for activity are known in the art. Once the core sequence of interest is identified, it is routine for one skilled in the art to obtain analogs and fragments.

Peptide Mimetics of H. pylori Polypeptides

The invention also provides for reduction of the protein binding domains of the subject H. pylori polypeptides to generate mimetics, e.g. peptide or non-peptide agents. The peptide mimetics are able to disrupt binding of a polypeptide to its counter ligand, e.g., in the case of an H. pylori polypeptide binding to a naturally occurring ligand. The critical residues of a subject H. pylori polypeptide which are involved in molecular recognition of a polypeptide can be determined and used to generate H. pylori-derived peptidomimetics which competitively or noncompetitively inhibit binding of the H. pylori polypeptide with an interacting polypeptide (see, for example, European patent applications EP-412,762A and EP-B31,080A).

For example, scanning mutagenesis can be used to map the amino acid residues of a particular H. pylori polypeptide involved in binding an interacting polypeptide, peptidomimetic compounds (e.g. diazepine or isoquinoline derivatives) can be generated which mimic those residues in binding to an interacting polypeptide, and which therefore can inhibit binding of an H. pylori polypeptide to an interacting polypeptide and thereby interfere with the function of H. pylori polypeptide. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gama lactam rings (Garvey et al. in Peptides. Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), and β-aminoalcohols (Gordon et al. (1985) Biochem Biophys Res Commun126:419; and Dann et al. (1986) Biochem Biophys Res Commun 134:71).

VI. Vaccine Formulations for H. pylori Nucleic Acids and Polypeptides

This invention also features vaccine compositions or formulations (used interchangeably herein) for protection against infection by H. pylori or for treatment of H. pylori infection. As used herein, the term “treatment of H. pylori infection” refers to therapeutic treatment of an existing or established H pylori infection. The terms “protection against H. pylori infection” or “prophylactic treatment” refer to the use of H. pylori vaccine formulation for reducing the risk of or preventing an infection in a subject at risk for H. pylori infection. In one embodiment, the vaccine compositions contain one or more immunogenic components, such as a surface protein, from H. pylori, or portion thereof, and a pharmaceutically acceptable carrier. For example, in one embodiment, the vaccine formulations of the invention contain at least one or combination of H. pylori polypeptides or fragments thereof, from same or different H. pylori antigens. Nucleic acids and H. pylori polypeptides for use in the vaccine formulations of the invention include the nucleic acids and polypeptides set forth in the Sequence Listing, preferably those H. pylori nucleic acids that encode surface proteins and surface proteins or fragments thereof. For example, a preferred nucleic acid and H. pylori polypeptide for use in a vaccine composition of the invention is selected from the group of nucleic acids which encode cell envelope proteins and H. pylori cell envelope proteins as set forth in Table 1. However, any nucleic acid encoding an immunogenic H. pylori protein and H. pylori polypetide, or portion thereof, can be used in the present invention. These vaccines have therapeutic and/or prophylactic utilities.

One aspect of the invention provides a vaccine composition for protection against infection by H. pylori which contains at least one immunogenic fragment of an H. pylori protein and a pharmaceutically acceptable carrier. Preferred fragments include peptides of at least about 10 amino acid residues in length, preferably about 10-20 amino acid residues in length, and more preferably about 12-16 amino acid residues in length.

Immunogenic components of the invention can be obtained, for example, by screening polypeptides recombinantly produced from the corresponding fragment of the nucleic acid encoding the full-length H. pylori protein. In addition, fragments can be chemically synthesized using techniques known in the art such as conventional Merrifield solid phase f-Moc or t-Boc chemistry.

In one embodiment, immunogenic components are identified by the ability of the peptide to stimulate T cells. Peptides which stimulate T cells, as determined by, for example, T cell proliferation or cytokine secretion are defined herein as comprising at least one T cell epitope. T cell epitopes are believed to be involved in initiation and perpetuation of the immune response to the protein allergen which is responsible for the clinical symptoms of allergy. These T cell epitopes are thought to trigger early events at the level of the T helper cell by binding to an appropriate HLA molecule on the surface of an antigen presenting cell, thereby stimulating the T cell subpopulation with the relevant T cell receptor for the epitope. These events lead to T cell proliferation, lymphokine secretion, local inflammatory reactions, recruitment of additional immune cells to the site of antigen/T cell interaction, and activation of the B cell cascade, leading to the production of antibodies. A T cell epitope is the basic element, or smallest unit of recognition by a T cell receptor, where the epitope comprises amino acids essential to receptor recognition (e.g., approximately 6 or 7 amino acid residues). Amino acid sequences which mimic those of the T cell epitopes are within the scope of this invention.

In another embodiment, immunogenic components of the invention are identified through genomic vaccination. The basic protocol is based on the idea that expression libraries consisting of all or parts of a pathogen genome, e.g., an H. pylori genome, can confer protection when used to genetically immunize a host. This expression library immunization (ELI) is analogous to expression cloning and involves reducing a genomic expression library of a pathogen, e.g., H. pylori, into plasmids that can act as genetic vaccines. The plasmids can also be designed to encode genetic adjuvants which can dramatically stimulate the humoral response. These genetic adjuvants can be introduced at remote sites and act as well extracelluraly as intracellularly.

This is a new approach to vaccine production that has many of the advantages of live/attenuated pathogens but no risk of infection. An expression library of pathogen DNA is used to immunize a host thereby producing the effects of antigen presentation of a live vaccine without the risk. For example, in the present invention, random fragments from the H. pylori genome or from cosmid or plasmid clones, as well as PCR products from genes identified by genomic sequencing, can be used to immunize a host. The feasibility of this approach has been demonstrated with Mycoplasma pulmonis (Barry et al., Nature 377:632-635, 1995), where even partial expression libraries of Mycoplasma pulnionis, a natural pathogen in rodents, provided protection against challenge from the pathogen.

ELI is a technique that allows for production of a non-infectious multipartite vaccine, even when little is known about pathogen's biology, because ELI uses the immune system to screen candidate genes. Once isolated, these genes can be used as genetic vaccines or for development of recombinant protein vaccines. Thus, ELI allows for production of vaccines in a systematic, largely mechanized fashion.

Screening immunogenic components can be accomplished using one or more of several different assays. For example, in vitro, peptide T cell stimulatory activity is assayed by contacting a peptide known or suspected of being immunogenic with an antigen presenting cell which presents appropriate MHC molecules in a T cell culture. Presentation of an immunogenic H. pylori peptide in association with appropriate MHC molecules to T cells in conjunction with the necessary costimulation has the effect of transmitting a signal to the T cell that induces the production of increased levels of cytokines, particularly of interleukin-2 and interleukin-4. The culture supernatant can be obtained and assayed for interleukin-2 or other known cytokines. For example, any one of several conventional assays for interleukin-2 can be employed, such as the assay described in Proc. Natl. Acad. Sci USA, 86: 1333 (1989) the pertinent portions of which are incorporated herein by reference. A kit for an assay for the production of interferon is also available from Genzyme Corporation (Cambridge, Mass.).

Alternatively, a common assay for T cell proliferation entails measuring tritiated thymidine incorporation. The proliferation of T cells can be measured in vitro by determining the amount of ³H-labeled thymidine incorporated into the replicating DNA of cultured cells. Therefore, the rate of DNA synthesis and, in turn, the rate of cell division can be quantified.

Vaccine compositions or formulations of the invention containing one or more immunogenic components (e.g., H. pylori polypeptide or fragment thereof or nucleic acid encoding an H. pylori polypeptide or fragment thereof) preferably include a pharmaceutically acceptable carrier. The term “pharmaceutically acceptable carrier” is intended to include any and all solvents, dispersion media, coatings, antibacterial and antifungal agents, isotonic and absorption delaying agents, and the like, compatible with pharmaceutical administration. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the H. pylori nucleic acid or polypeptide. For vaccine formulations of the invention containing H. pylori polypeptides, the polypeptide is preferably coadministered with a suitable adjuvant and/or a delivery system described herein.

It will be apparent to those of skill in the art that the therapeutically effective amount of DNA or protein of this invention will depend, inter alia, upon the administration schedule, the unit dose of an H. pylori nucleic acid or polypeptide administered, whether the protein or nucleic acid is administered in combination with other therapeutic agents, the immune status and health of the patient, and the therapeutic activity of the particular protein or nucleic acid.

Vaccine formulations are conventionally administered parenterally, e.g., by injection, either subcutaneously or intramuscularly. Methods for intramuscular immunization are described by Wolff et al. (1990) Science 247: 1465-1468 and by Sedegah et al. (1994) Immunology 91: 9866-9870. Other modes of administration include oral and pulmonary formulations, suppositories, and transdermal applications. Oral immunization is preferred over parenteral methods for inducing protection against infection by H. pylori. Czinn et. al. (1993) Vaccine 11: 637-642. Oral formulations include such normally employed excipients as, for example, pharmaceutical grades of mannitol, lactose, starch, magnesium stearate, sodium saccharine, cellulose, magnesium carbonate, and the like.

In one embodiment, the vaccine formulation includes, as a pharmaceutically acceptable carrier, an adjuvant. Examples of the suitable adjuvants for use in the vaccine formulations of the invention include, but are not limited, to aluminum hydroxide; N-acetyl-muramyl—L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3-hydroxyphos-phoryloxy)-ethylamine (CGP 19835A, referred to a MTP-PE); RIBI, which contains three components from bacteria; monophosphoryl lipid A; trehalose dimycoloate; cell wall skeleton (MPL+TDM+CWS) in a 2% squalene/Tween 80 emulsion; and cholera toxin. Others which may be used are non-toxic derivatives of cholera toxin, including its B subunit, and/or conjugates or genetically engineered fusions of the H. pylori polypeptide with cholera toxin or its B subunit, procholeragenoid, fungal polysaccharides, including schizophyllan, muramyl dipeptide, muramyl dipeptide derivatives, phorbol esters, labile toxin of E. coli, non-H. pylori bacterial lysates, block polymers or saponins.

In another embodiment, the vaccine formulation includes, as a pharmaceutically acceptable carrier, a delivery system. Suitable delivery systems for use in the vaccine formulations of the invention include biodegradable microcapsules or immuno-stimulating complexes (ISCOMs), cochleates, or liposomes, genetically engineered attenuated live vectors such as viruses or bacteria, and recombinant (chimeric) virus-like particles, e.g., bluetongue. In another embodiment of the invention, the vaccine formulation includes both a delivery system and an adjuvant.

Delivery systems in humans may include enteric release capsules protecting the antigen from the acidic environment of the stomach, and including H. pylori polypeptide in an insoluble form as fusion proteins. Suitable carriers for the vaccines of the invention are enteric coated capsules and polylactide-glycolide microspheres. Suitable diluents are 0.2 N NaHCO3 and/or saline.

Vaccines of the invention can be administered as a primary prophylactic agent in adults or in children, as a secondary prevention, after successful eradication of H. pylori in an infected host, or as a therapeutic agent in the aim to induce an immune response in a susceptible host to prevent infection by H. pylori. The vaccines of the invention are administered in amounts readily determined by persons of ordinary skill in the art. Thus, for adults a suitable dosage will be in the range of 10 μg to 10 g, preferably 10 μg to 100 mg, for example 50 μg to 50 mg. A suitable dosage for adults will also be in the range of 5 μg to 500 mg. Similar dosage ranges will be applicable for children.

The amount of adjuvant employed will depend on the type of adjuvant used. For example, when the mucosal adjuvant is cholera toxin, it is suitably used in an amount of 5 μg to 50 μg, for example 10 μg to 35 μg. When used in the form of microcapsules, the amount used will depend on the amount employed in the matrix of the microcapsule to achieve the desired dosage. The determination of this amount is within the skill of a person of ordinary skill in the art.

Those skilled in the art will recognize that the optimal dose may be more or less depending upon the patient's body weight, disease, the route of administration, and other factors. Those skilled in the art will also recognize that appropriate dosage levels can be obtained based on results with known oral vaccines such as, for example, a vaccine based on an E. coli lysate (6 mg dose daily up to total of 540 mg) and with an enterotoxigenic E. coli purified antigen (4 doses of 1 mg) (Schulman et al., J. Urol. 150:917-921 (1993)); Boedecker et al., American Gastroenterological Assoc. 999:A-222 (1993)). The number of doses will depend upon the disease, the formulation, and efficacy data from clinical trials. Without intending any limitation as to the course of treatment, the treatment can be administered over 3 to 8 doses for a primary immunization schedule over 1 month (Boedeker, American Gastroenterological Assoc. 888:A-222 (1993)).

In a preferred embodiment, a vaccine composition of the invention can be based on a killed whole E coli preparation with an immunogenic fragment of an H. pylori protein of the invention expressed on its surface or it can be based on an E. coli lysate, wherein the killed E. coli acts as a carrier or an adjuvant.

It will be apparent to those skilled in the art that some of the vaccine 4 compositions of the invention are useful only for preventing H. pylori infection, some are useful only for treating H. pylori infection, and some are useful for both preventing and treating H. pylori infection. In a preferred embodiment, the vaccine composition of the invention provides protection against H. pylori infection by stimulating humoral and/or cell-mediated immunity against H. pylori. It should be understood that amelioration of any of the symptoms of H. pylori infection is a desirable clinical goal, including a lessening of the dosage of medication used to treat H. pylori-caused disease, or an increase in the production of antibodies in the serum or mucous of patients.

VII. Antibodies Reactive With H. pylori Polypeptides

The invention also includes antibodies specifically reactive with the subject H. pylori polypeptide. Anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal such as a mouse, a hamster or rabbit can be immunized with an immunogenic form of the peptide. Techniques for conferring immunogenicity on a protein or peptide include conjugation to carriers or other techniques well known in the art. An immunogenic portion of the subject H. pylori polypeptide can be administered in the presence of adjuvant. The progress of immunization can be monitored by detection of antibody titers in plasma or serum. Standard ELISA or other immunoassays can be used with the immunogen as antigen to assess the levels of antibodies.

In a preferred embodiment, the subject antibodies are immunospecific for antigenic determinants of the H. pylori polypeptides of the invention, e.g. antigenic determinants of a polypeptide of the invention contained in the Sequence Listing, or a closely related human or non-human mammalian homolog (e.g., 90% homologous, more preferably at least 95% homologous). In yet a further preferred embodiment of the invention, the anti-H. pylori antibodies do not substantially cross react (i.e., react specifically) with a protein which is for example, less than 80% percent homologous to a sequence of the invention contained in the Sequence Listing. By “not substantially cross react”, it is meant that the antibody has a binding affinity for a non-homologous protein which is less than 10 percent, more preferably less than 5 percent, and even more preferably less than 1 percent, of the binding affinity for a protein of the invention contained in the Sequence Listing. In a most preferred embodiment, there is no crossreactivity between bacterial and mammalian antigens.

The term antibody as used herein is intended to include fragments thereof which are also specifically reactive with H. pylori polypeptides. Antibodies can be fragmented using conventional techniques and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments. The antibody of the invention is further intended to include bispecific and chimeric molecules having an anti-H. pylori portion.

Both monoclonal and polyclonal antibodies (Ab) directed against H. pylori polypeptides or H. pylori polypeptide variants, and antibody fragments such as Fab′ and F(ab′)₂, can be used to block the action of H. pylori polypeptide and allow the study of the role of a particular H. pylori polypeptide of the invention in aberrant or unwanted intracellular signaling, as well as the normal cellular function of the H. pylori and by microinjection of anti-H. pylori polypeptide antibodies of the present invention.

Antibodies which specifically bind H. pylori epitopes can also be used in immunohistochemical staining of tissue samples in order to evaluate the abundance and pattern of expression of H. pylori antigens. Anti H. pylori polypeptide antibodies can be used diagnostically in immuno-precipitation and immuno-blotting to detect and evaluate H. pylori levels in tissue or bodily fluid as part of a clinical testing procedure. Likewise, the ability to monitor H. pylori polypeptide levels in an individual can allow determination of the efficacy of a given treatment regimen for an individual afflicted with such a disorder. The level of an H. pylori polypeptide can be measured in cells found in bodily fluid, such as in urine samples or can be measured in tissue, such as produced by gastric biopsy. Diagnostic assays using anti-H. pylori antibodies can include, for example immunoassays designed to aid in early diagnosis of H. pylori infections. The present invention can also be used as a method of detecting antibodies contained in samples from individuals infected by this bacterium using specific H. pylori antigens.

Another application of anti-H. pylori polypeptide antibodies of the invention is in the immunological screening of cDNA libraries constructed in expression vectors such as λgt11, λgt18-23, λZAP, and λORF8. Messenger libraries of this type, having coding sequences inserted in the correct reading frame and orientation, can produce fusion proteins. For instance, λgt11 will produce fusion proteins whose amino termini consist of β-galactosidase amino acid sequences and whose carboxy termini consist of a foreign polypeptide. Antigenic epitopes of a subject H. pylori polypeptide can then be detected with antibodies, as, for example, reacting nitrocellulose filters lifted from infected plates with anti-H. pylori polypeptide antibodies. Phage, scored by this assay, can then be isolated from the infected plate. Thus, the presence of H. pylori gene homologs can be detected and cloned from other species, and alternate isoforms (including splicing variants) can be detected and cloned.

VIII. Kits Containing Nucleic Acids, Polypeptides or Antibodies of the Invention

The nucleic acid, polypeptides and antibodies of the invention can be combined with other reagents and articles to form kits. Kits for diagnostic purposes typically comprise the nucleic acid, polypeptides or antibodies in vials or other suitable vessels. Kits typically comprise other reagents for performing hybridization reactions, polymerase chain reactions (PCR), or for reconstitution of lyophilized components, such as aqueous media, salts, buffers, and the like. Kits may also comprise reagents for sample processing such as detergents, chaotropic salts and the like. Kits may also comprise immobilization means such as particles, supports, wells, dipsticks and the like. Kits may also comprise labeling means such as dyes, developing reagents, radioisotopes, fluorescent agents, luminescent or chemiluminescent agents, enzymes, intercalating agents and the like. With the nucleic acid and amino acid sequence information provided herein, individuals skilled in art can readily assemble kits to serve their particular purpose. Kits further can include instructions for use.

IX. Drug Screening Assays Using H. pylori Polypeptides

By making available purified and recombinant H. pylori polypeptides, the present invention provides assays which can be used to screen for drugs which are either agonists or antagonists of the normal cellular function, in this case, of the subject H. pylori polypeptides, or of their role in intracellular signaling. Such inhibitors or potentiators may be useful as new therapeutic agents to combat H. pylori infections in humans. A variety of assay formats will suffice and, in light of the present inventions, will be comprehended by the skilled artisan.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or change in enzymatic properties of the molecular target. Accordingly, in an exemplary screening assay of the present invention, the compound of interest is contacted with an isolated and purified H. pylori polypeptide.

Screening assays can be constructed in vitro with a purified H. pylori polypeptide or fragment thereof, such as an H. pylori polypeptide having enzymatic activity, such that the activity of the polypeptide produces a detectable reaction product. The efficacy of the compound can be assessed by generating dose response curves from data obtained using various concentrations of the test compound. Moreover, a control assay can also be performed to provide a baseline for comparison. Suitable products include those with distinctive absorption, fluorescence, or chemi-luminescence properties, for example, because detection may be easily automated. A variety of synthetic or naturally occurring compounds can be tested in the assay to identify those which inhibit or potentiate the activity of the H. pylori polypeptide. Some of these active compounds may directly, or with chemical alterations to promote membrane permeability or solubility, also inhibit or potentiate the same activity (e.g., enzymatic activity) in whole, live H. pylori cells.

This invention is further illustrated by the following examples which should not be construed as limiting. The contents of all references and published patent applications cited throughout this application are hereby incorporated by reference.

### EXEMPLIFICATION

I. Cloning and Sequencing of H. pylori DNA

H. pylori chromosomal DNA was isolated according to a basic DNA protocol outlined in Schleif R. F. and Wensink P. C., Practical Methods in Molecular Biology, p.98, Springer-Verlag, N.Y., 1981, with minor modifications. Briefly, cells were pelleted, resuspended in TE (10 mM Tris, 1 mM EDTA, pH 7.6) and GES lysis buffer (5.1 M guanidium thiocyanate, 0.1 M EDTA, pH 8.0, 0.5% N-laurylsarcosine) was added. Suspension was chilled and ammonium acetate (NH₄Ac) was added to final concentration of 2.0 M. DNA was extracted, first with chloroform, then with phenol-chloroform, and reextracted with chloroform. DNA was precipitated with isopropanol, washed twice with 70% EtOH, dried and resuspended in TE.

Following isolation whole genomic H. pylori DNA was nebulized (Bodenteich et al., Automated DNA Sequencing and Analysis (J. C. Venter, ed.), Academic Press, 1994) to a median size of 2000 bp. After nebulization, the DNA was concentrated and separated on a standard 1% agarose gel. Several fractions, corresponding to approximate sizes 900-1300 bp, 1300-1700 bp, 1700-2200 bp, 2200-2700 bp, were excised from the gel and purified by the GeneClean procedure (Bio101, Inc.).

The purified DNA fragments were then blunt-ended using T4 DNA polymerase. The healed DNA was then ligated to unique BstXI-linker adapters in 100-1000 fold molar excess. These linkers are complimentary to the BstXI-cut pMPX vectors, while the overhang is not self-complimentary. Therefore, the linkers will not concatemerize nor will the cut-vector religate itself easily. The linker-adopted inserts were separated from the unincorporated linkers on a 1% agarose gel and purified using GeneClean. The linker-adopted inserts were then ligated to each of the 20 pMPX vectors to construct a series of “shotgun” subclone libraries. The vectors contain an out-of-frame lacZ gene at the cloning site which becomes in-frame in the event that an adapter-dimer is cloned, allowing these to be avoided by their blue-color.

All subsequent steps were based on the multiplex DNA sequencing protocols outlined in Church G. M. and Kieffer-Higgins S., Science 240:185-188, 1988. Only major modifications to the protocols are highlighted. Briefly, each of the 20 vectors was then transformed into DH5α competent cells (Gibco/BRL, DH5α transformation protocol). The libraries were assessed by plating onto antibiotic plates containing ampicillin, methicillin and IPTG/Xgal. The plates were incubated overnight at 37° C. Successful transformants were then used for plating of clones and pooling into the multiplex pools. The clones were picked and pooled into 40 ml growth medium cultures. The cultures were grown overnight at 37° C. DNA was purified using the Qiagen Midi-prep kits and Tip-100 columns (Qiagen, Inc.). In this manner, 100 μg of DNA was obtained per pool. Fifteen 96-well plates of DNA were generated to obtain a 5-10 fold sequence redundancy assuming 250-300 base average read-lengths.

These purified DNA samples were then sequenced using the multiplex DNA sequencing based on chemical degradation methods (Church G. M. and Kieffer-Higgins S., Science 240:185-188, 1988) or by Sequthrem (Epicenter Technologies) dideoxy sequencing protocols. The sequencing reactions were electrophoresed and transferred onto nylon membranes by direct transfer electrophoresis from 40 cm gels (Richterich P. and Church G. M., Methods in Enzymology 218:187-222, 1993) or by electroblotting (Church, supra). 24 samples were run per gel. 45 successful membranes were produced by chemical sequencing and 8 were produced by dideoxy sequencing. The DNA was covalently bound to the membranes by exposure to ultraviolet light, and hybridized with labeled oligonucleotides complimentary to tag sequences on the vectors (Church, supra). The membranes were washed to rinse off non-specifically bound probe, and exposed to X-ray film to visualize individual sequence ladders. After autoradiography, the hybridized probe was removed by incubation at 65° C., and the hybridization cycle repeated with another tag sequence until the membrane had been probed 38 times for chemical sequencing membranes and 10 times for the dideoxy sequencing membranes. Thus, each gel produced a large number of films, each containing new sequencing information. Whenever a new blot was processed, it was initially probed for an internal standard sequence added to each of the pools.

Digital images of the films were generated using a laser-scanning densitometer (Molecular Dynamics, Sunnyvale, Calif.). The digitized images were processed on computer workstations (VaxStation 4000's) using the program REPLICA™ (Church et al., Automated DNA Sequencing and Analysis (J. C. Venter, ed.), Academic Press, 1994). Image processing included lane straightening, contrast adjustment to smooth out intensity differences, and resolution enhancement by iterative gaussian deconvolution. The sequences were then automatically picked in REPLICA™ and displayed for interactive proofreading before being stored in a project database. The proofreading was accomplished by a quick visual scan of the film image followed by mouse clicks on the bands of the displayed image to modify the base calls. Many of the sequence errors could be detected and corrected because multiple sequence reads covering the same portion of the genomic DNA provide adequate sequence redundancy for editing. Each sequence automatically received an identification number (corresponding to microtiter plate, probe information, and lane set number). This number serves as a permanent identifier of the sequence so it is always possible to identify the original of any particular sequence without recourse to a specialized database.

Routine assembly of H. pylori sequences was done using the program FALCON (Church, Church et al., Automated DNA Sequenicng and Analysis (J. C. Venter, ed.), Academic Press, 1994). This program has proven to be fast and reliable for most sequences. The assembled contigs were displayed using a modified version of GelAssemble, developed by the Genetics Computer Group (GCG) (Devereux et al., Nucleic Acid Res. 12:387-95, 1984) that interacts with REPLICA™. This provided for an integrated editor that allows multiple sequence gel images to be instantaneously called up from the REPLICA™ database and displayed to allow rapid scanning of contigs and proofreading of gel traces where discrepancies occurred between different sequence reads in the assembly.

II. Identification, Cloning and Expression of recombinant H. pylori DNA Sequences

To facilitate the cloning, expression and purification of membrane and secreted proteins from H. pylori a powerful gene expression system, the pET System (Novagen), for cloning and expression of recombinant proteins in E. coli, was selected. Also, a DNA sequence encoding a peptide tag, the His-Tag, was fused to the 3′ end of DNA sequences of interest in order to facilitate purification of the recombinant protein products. The 3′ end was selected for fusion in order to avoid alteration of any 5′ terminal signal sequence. The exception to the above was ppiB, a gene cloned for use as a control in the expression studies. In this study, the sequence for H. pylori ppiB contains a DNA sequence encoding a His-Tag fused to the 5′ end of the full length gene, because the protein product of this gene does not contain a signal sequence and is expressed as a cytosolic protein.

PCR Amplification and Cloning of DNA Sequences Containing ORF's for Membrane and Secreted Proteins from the J99 Strain of Helicobacter pylori.

Sequences chosen (from the list of the DNA sequences of the invention) for cloning from the J99 strain of H. pylori were prepared for amplification cloning by polymerase chain reaction (PCR). Synthetic oligonucleotide primers (Table 3) specific for the 5′ and 3′ ends of open reading frames (ORFs) were designed and purchased (GibcoBRL Life Technologies, Gaithersburg, Md., USA). All forward primers (specific for the 5′ end of the sequence) were designed to include an NcoI cloning site at the extreme 5′ terminus, except for HpSeq. 4821082 (SEQ ID NO: 9730) where NdeI was used. These primers were designed to permit initiation of protein translation at a methionine residue followed by a valine residue and the coding sequence for the remainder of the native H. pylori DNA sequence. An exception is H. pylori sequence 4821082 (SEQ ID NO: 9730) where the initiator methionine is immediately followed by the remainder of the native H. pylori DNA sequence. All reverse primers (specific for the 3′ end of any H. pylori ORF) included a EcoRI site at the extreme 5′ terminus to permit cloning of each H. pylori sequence into the reading frame of the pET-28b. The pET-28b vector provides sequence encoding an additional 20 carboxy-terminal amino acids (only 19 amino acids in HpSeq. 26380318 (SEQ ID NO: 4851) and HpSeq.14640637 (SEQ ID NO: 8015) including six histidine residues (at the extreme C-terminus), which comprise the His-Tag. An exception to the above, as noted earlier, is the vector construction for the ppiB gene. A synthetic oligonucleotide primer specific for the 5′ end of ppiB gene encoded a BamHI site at its extreme 5′ terminus and the primer for the 3′ end of the ppiB gene encoded a XhoI site at its extreme 5′ terminus.

TABLE 3

Oligonucleotide primers used for PCR amplification of H pylon DNA se-
quences

| | |
|---|---|
| Forward primer 5′ to 3′ | Reverse Primer 5′ to 3′ |

| | | |
|---|---|---|
| Outer membrane | | |
| Proteins | | |
| Protein 16225006 | 5′-TATACCATGGTGGG | 5′- |
| (aa SEQ ID NO: 9642) | CGCTAA-3′(SEQ ID | ATGAATTCGAGTAAG |
| (nt SEQ ID NO: 9530) | NO:9749) | GATTTTTG-3′ (SEQ ID |
| | | NO:9750) |
| Protein 26054702 | 5′- | 5′- |
| (aa SEQ ID NO: 9450) | TTAACCATGGTGAAA | TAGAATTCGCATAAC |
| (nt SEQ ID NO: 4688) | AGCGATA-3′ (SEQ ID | GATCAATC-3′ (SEQ ID |
| | NO:9751) | NO:9752) |
| Protein 7116626 | 5′- | 5′- |
| (aa SEQ ID NO: 9048) | ATATCCATGGTGAGT | ATGAATTCAATTTTT |
| (nt SEQ ID NO: 4286) | TTGATGA-3′ (SEQ ID | TATTTTGCCA-3′ (SEQ |
| | NO:9753) | ID NO:9754) |
| Protein 29479681 | 5′- | 5′- |
| (aa SEQ ID NO: 5379) | AATTCCATGGTGGGG | ATGAATTCTCGATAG |
| (nt SEQ ID NO: 617) | GCTATG-3′ (SEQ ID | CCAAAATC-3′ (SEQ ID |
| | NO:9755) | NO:9756) |
| Protein 14640637 | 5′- | 5′- |
| (aa SEQ ID NO: 8015) | AATTCCATGGTGCAT | AAGAATTCTCTAGCA |
| (nt SEQ ID NO: 3253) | AACTTCCATT-3′ (SEQ | TCCAAATGGA-3′ (SEQ |
| | ID NO:9757) | ID NO:9758) |
| Periplasmic/Secreted | | |
| Proteins | | |
| Protein 30100332 | 5′-ATTTCCATGGTCATG | 5′- |
| (aa SEQ ID NO: 5933) | TCTCATATT-3′ (SEQ ID | ATGAATTCCATCTTT |
| (nt SEQ ID NO: 1171) | NO:9759) | TATTCCAC-3′ (SEQ ID |
| | | NO:9760) |
| Protein 4721061 | 5′-AACCATGGTGATTT | 5′- |
| (aa SEQ ID NO: 5181) | TAAGCATTGAAAG-3′ | AAGAATTCCACTCA |
| (nt SEQ ID NO: 419) | (SEQ ID NO:9761) | AAATTTTTTAACAG-3′ |
| | | (SEQ ID NO:9762) |
| Other Surface Proteins | | |
| Protein 4821082 | 5′-GATCATCCATATGTT | 5′- |
| (aa SEQ ID NO: 9730) | ATCTTCTAAT-3′ (SEQ | TCAATCAACCATTT |
| (nt SEQ ID NO: 9618 | ID NO:9763) | TAACCCTG-3′ (SEQ ID |
| | | NO:9764) |
| Protein 978477 | 5′-TATACCATGGTGAA | 5′- |
| (aa SEQ ID NO: 5231) | ATTTTTTCTTTTA-3′ | AGAATTCAATTGCG |
| (nt SEQ ID NO: 469) | (SEQ ID NO:9765) | TCTTGTAAAAG-3′ |
| | | (SEQ ID NO:9766) |
| Inner Membrane | | |
| Protein | | |
| Protein 26380318 | 5′-TATACCATGGTGAT | 5′-ATGAATTCCCACTT |
| (aa SEQ ID NO: 4851) | GGACAAACTC-3′ (SEQ | GGGGCGATA-3′ (SEQ |
| (nt SEQ ID NO: 89) | ID NO:9767) | ID NO:9768) |
| Cytoplasmic Protein | | |
| ppi | 5′-TTATGGATCCAAAC | 5′-TATCTCGAGTTATA |
| (aa SEQ ID NO: 7674) | CAATTAAAACT-3′ (SEQ | GAGAAGGGC-3′ (SEQ |
| (nt SEQ ID NO: 2912) | ID NO:9769) | ID NO:9770) |

Genomic DNA prepared from the J99 strain of H. pylori (ATCC #55679; deposited by Genome Therapeutics Corporation, 100 Beaver Street, Waltham, Mass. 02154) was used as the source of template DNA for PCR amplification reactions (Current Protocols in Molecular Biology, John Wiley and Sons, Inc., F. Ausubel et al., eds., 1994). To amplify a DNA sequence containing an H. pylori ORF, genomic DNA (50 nanograms) was introduced into a reaction vial containing 2 mM MgCl₂, 1 micromolar synthetic oligonucleotide primers (forward and reverse primers) complementary to and flanking a defined H. pylori ORF, 0.2 mM of each deoxynucleotide triphosphate; dATP, dGTP, dCTP, dTTP and 2.5 units of heat stable DNA polymerase (Amplitaq, Roche Molecular Systems, Inc., Branchburg, N.J., USA) in a final volume of 100 microliters. The following thermal cycling conditions were used to obtain amplified DNA products for each ORF using a Perkin Elmer Cetus/GeneAmp PCR System 9600 thermal cycler:

Protein 26054702 (SEQ ID NO: 9450), Protein 7116626 (SEQ ID NO: 9048), Protein 29479681 (SEQ ID NO: 5379), Protein 30100332 (SEQ ID NO: 5933), and Protein 4821082(SEQ ID NO: 9730);

Denaturation at 94° C. for 2 min,

2 cycles at 94° C. for 15 sec, 30° C. for 15 sec and 72° C. for 1.5 min

23 cycles at 94° C. for 15 sec, 55° C. for 15 sec and 72° C. for 1.5 min

Reactions were concluded at 72° C. for 6 minutes.

Protein 16225006 (SEQ ID NO: 9642);

Denaturation at 94° C. for 2 min,

25 cycles at 95° C. for 15 sec, 55° C. for 15 sec and 72° C. for 1.5 min

Reaction was concluded at 72° C. for 6 minutes.

Protein 4721061 (SEQ ID NO: 5181);

Denaturation at 94° C. for 2 min,

2 cycles at 94° C. for 15 sec, 36° C. for 15 sec and 72° C. for 1.5 min

23 cycles at 94° C. for 15 sec, 60° C. for 15 sec and 72° C. for 1.5 min

Reactions were concluded at 72° C. for 6 minutes.

Protein 26380318(SEQ ID NO: 4851);

Denaturation at 94° C. for 2 min,

2 cycles at 94° C. for 15 sec, 38° C. for 15 sec and 72° C. for 1.5 min

23 cycles at 94° C. for 15 sec, 62° C. for 15 sec and 72° C. for 1.5 min

Reactions were concluded at 72° C. for 6 minutes.

Protein 14640637 (SEQ ID NO: 8015);

Denaturation at 94° C. for 2 min,

2 cycles at 94° C. for 15 sec, 33° C. for 15 sec and 72° C. for 1.5 min

30 cycles at 94° C. for 15 sec, 55° C. for 15 sec and 72° C. for 1.5 min

Reactions were concluded at 72° C. for 6 minutes.

Conditions for amplification of H. pylori ppiB (SEQ ID NO: 7674);

Denaturation at 94° C. for 2 min,

2 cycles at 94° C. for 15 sec, 32° C. for 15 sec and 72° C. for 1.5 min

25 cycles at 94° C. for 15 sec, 56° C. for 15 sec and 72° C. for 1.5 min

Reactions were concluded at 72° C. for 6 minutes

Upon completion of thermal cycling reactions, each sample of amplified DNA was washed and purified using the Qiaquick Spin PCR purification kit (Qiagen, Gaithersburg, Md., USA). All amplified DNA samples were subjected to digestion with the restriction endonucleases, NcoI and EcoRI (New England BioLabs, Beverly, Mass., USA), or in the case of HpSeq. 4821082 (SEQ ID NO: 9730), with NdeI and EcoRI (Current Protocols in Molecular Biology, John Wiley and Sons, Inc., F. Ausubel et al., eds., 1994). DNA samples were then subjected to electrophoresis on 1.0% NuSeive (FMC BioProducts, Rockland, Me. USA) agarose gels. DNA was visualized by exposure to ethidium bromide and long wave uv irradiation. DNA contained in slices isolated from the agarose gel was purified using the Bio 101 GeneClean Kit protocol (Bio 101 Vista, Calif., USA).

Cloning of H. pylori DNA Sequences into the pET-28b Prokaryotic Expression Vector.

The pET-28b vector-was prepared for cloning by digestion with NcoI and EcoRI, or in the case of H. pylori protein 4821082 (SEQ ID NO: 9730) with NdeI and EcoRI (Current Protocols in Molecular Biology, John Wiley and Sons, Inc., F. Ausubel et al., eds., 1994). In the case of cloning ppiB, the pET-28a vector, which encodes a His-Tag that can be fused to the 5′ end of an inserted gene, was used and the cloning site prepared for cloning with the ppiB gene by digestion with BamHI and XhoI restriction endonucleases.

Following digestion, DNA inserts were cloned (Current Protocols in Molecular Biology, John Wiley and Sons, Inc., F. Ausubel et al., eds., 1994) into the previously digested pET-28b expression vector, except for the amplified insert for ppiB, which was cloned into the pET-28a expression vector. Products of the ligation reaction were then used to transform the BL21 strain of E. coli (Current Protocols in Molecular Biology, John Wiley and Sons, Inc., F. Ausubel et al., eds., 1994) as described below.

Transformation of Competent Bacteria with Recombinant Plasmids

Competent bacteria, E coli strain BL21 or E. coli strain BL21 (DE3), were transformed with recombinant pET expression plasmids carrying the cloned H. pylori sequences according to standard methods (Current Protocols in Molecular, John Wiley and Sons, Inc., F. Ausubel et al., eds., 1994). Briefly, 1 microliter of ligation reaction was mixed with 50 microliters of electrocompetent cells and subjected to a high voltage pulse, after which, samples were incubated in 0.45 milliliters SOC medium (0.5% yeast extract, 2.0% tryptone, 10 mM NaCl, 2.5 mM KCl, 10 mM MgCl2, 10 mM MgSO4 and 20, mM glucose) at 37° C. with shaking for 1 hour. Samples were then spread on LB agar plates containing 25 microgram/ml kanamycin sulfate for growth overnight. Transformed colonies of BL21 were then picked and analyzed to evaluate cloned inserts as described below.

Identification of Recombinant pET Expression Plasmids Carrying H. pylori Sequences

Individual BL21 clones transformed with recombinant pET-28b-H. pylori ORFs were analyzed by PCR amplification of the cloned inserts using the same forward and reverse primers, specific for each H. pylori sequence, that were used in the original PCR amplification cloning reactions. Successful amplification verified the integration of the H. pylori sequences in the expression vector (Current Protocols in Molecular Biology, John Wiley and Sons, Inc., F. Ausubel et al., eds., 1994).

Isolation and Preparation of Plasmid DNA from BL21 Transformants

Individual clones of recombinant pET-28b vectors carrying properly cloned H. pylori ORFs were picked and incubated in 5 mls of LB broth plus 25 microgram/ml kanamycin sulfate overnight. The following day plasmid DNA was isolated and purified using the Qiagen plasmid purification protocol (Qiagen Inc., Chatsworth, Calif., USA).

Expression of Recombinant H. pylori Sequences in E. coli

The pET vector can be propagated in any E. coli K-12 strain e.g. HMS 174, HB 101, JM 109, DH5, etc. for the purpose of cloning or plasmid preparation. Hosts for expression include E. coli strains containing a chromosomal copy of the gene for T7 RNA polymerase. These hosts are lysogens of bacteriophage DE3, a lambda derivative that carries the lacI gene, the lacUV5 promoter and the gene for T7 RNA polymerase. T7 RNA polymerase is induced by addition of isopropyl-B-D-thiogalactoside (IPTG), and the T7 RNA polymerase transcribes any target plasmid, such as pET-28b, carrying a T7 promoter and a gene of interest. Strains used include: BL21(DE3) (Studier, F. W., Rosenberg, A. H., Dunn, J. J., and Dubendorff, J. W. (1990) Meth. Enzymol. 185, 60-89).

To express recombinant H. pylori sequences, 50 nanograms of plasmid DNA isolated as described above was used to transform competent BL21 (DE3) bacteria as described above (provided by Novagen as part of the pET expression system kit). The lacZ gene (beta-galactosidase) was expressed in the pET-System as described for the H. pylori recombinant constructions. Transformed cells were cultured in SOC medium for 1 hour, and the culture was then plated on LB plates containing 25 micrograms/ml kanamycin sulfate. The following day, bacterial colonies were pooled and grown in LB medium containing kanamycin sulfate (25 micrograms/ml) to an optical density at 600 nM of 0.5 to 1.0 O.D. units, at which point, 1 millimolar IPTG was added to the culture for 3 hours to induce gene expression of the H. pylori recombinant DNA constructions.

After induction of gene expression with IPTG, bacteria were pelleted by centrifugation in a Sorvall RC-3B centrifuge at 3500×g for 15 minutes at 4° C. Pellets were resuspended in 50 milliliters of cold 10 mM Tris-HCl, pH 8.0, 0.1 M NaCl and 0.1 mM EDTA (STE buffer). Cells were then centrifuged at 2000×g for 20 min at 4° C. Wet pellets were weighed and frozen at −80° C. until ready for protein purification.

III. Purification of Recombinant Proteins from E. coli

Analytical Methods

The concentrations of purified protein preparations were quantified spectrophotometrically using absorbance coefficients calculated from amino acid content (Perkins, S. J. 1986 Eur. J. Biochem. 157, 169-180). Protein concentrations were also measured by the method of Bradford, M. M. (1976) Anal. Biochem. 72, 248-254, and Lowry, O. H., Rosebrough, N., Farr, A. L. & Randall, R. J. (1951) J. Biol. Chem. 193, pages 265-275, using bovine serum albumin as a standard.

SDS-polyacrylamide gels (12% or 4.0 to 25% acrylamide gradient gels) were purchased from BioRad (Hercules, Calif., USA), and stained with Coomassie blue. Molecular weight markers included rabbit skeletal muscle myosin (200 kDa), E. coli (-galactosidase (116 kDa), rabbit muscle phosphorylase B (97.4 kDa), bovine serum albumin (66.2 kDa), ovalbumin (45 kDa), bovine carbonic anhydrase (31 kDa), soybean trypsin inhibitor (21.5 kDa), egg white lysozyme (14.4 kDa) and bovine aprotinin (6.5 kDa).

1. Purification of Soluble Proteins

All steps were carried out at 4° C. Frozen cells were thawed, resuspended in 5 volumes of lysis buffer (20 mM Tris, pH 7.9, 0.5 M NaCl, 5 mM imidazole with 10% glycerol, 0.1% 2-mercaptoethanol, 200 μg/ml lysozyme, 1 mM phenylmethylsulfonyl fluoride (PMSF), and 10 ug/ml each of leupeptin, aprotinin, pepstatin, L-1-chloro-3-[4-tosylamido]-7-amino-2-heptanone (TLCK), L-1-chloro-3-[4-tosylamido]-4-phenyl-2-butanone (TPCK), and soybean trypsin inhibitor, and ruptured by several passages through a small volume microfluidizer (Model M-110S, Microfluidics International Corporation, Newton, Mass.). The resultant homogenate was made 0.1% Brij 35, and centrifuged at 100,000×g for 1 hour to yield a clear supernatant (crude extract).

Following filtration through a 0.8 μm Supor filter (Gelman Sciences, FRG) the crude extract was loaded directly onto a Ni²⁺-nitrilotriacetate-agarose (NTA) with a 5 milliliter bed volume (Hochuli, E., Dbeli, H., and Schacheer, A. (1987) J. Chromatography 411, 177-184) pre-equilibrated in lysis buffer containing 10% glycerol, 0.1% Brij 35 and 1 mM PMSF. The column was washed with 250 ml (50 bed volumes) of lysis buffer containing 10% glycerol, 0.1% Brij 35, and was eluted with sequential steps of lysis buffer containing 10% glycerol, 0.05% Brij 35, 1 mM PMSF, and 20, 100, 200, and 500 mM imidazole in succession. Fractions were monitored by absorbance at OD₂₈₀ nm, and peak fractions were analyzed by SDS-PAGE. Fractions containing the recombinant protein eluted at 100 mM imidazole.

Recombinant Protein 1-, 5-0637 (SEQ ID NO: 8015) and Proteins, Beta-Galactosidase (lacZ) and Peptidyl-prolyl Cis-trans Isomerase (ppiB SEQ ID NO: 7674)

Fractions containing the recombinant proteins from the Ni²⁺-NTA-agarose columns were pooled and then concentrated to approximately 5 ml by centrifugal filtration (Centriprep-10, Amicon, Mass.), and loaded directly onto a 180-ml column (1.6×91 cm) of Sephacryl S-100 HR gel filtration medium equilibrated in Buffer A (10 mM Hepes, pH 7.5, 150 mM NaCl, 0.1 mM EGTA) and run in Buffer A at 18 ml/h. Fractions containing the recombinant protein were identified by absorbance at 280 nm and analyzed by SDS-PAGE. Fractions were pooled and concentrated by centrifugal filtration.

Recombinant Protein 7116626 (SEQ ID NO: 9048)

Fractions containing the recombinant protein from the Ni²⁺-NTA-agarose column were pooled and dialyzed overnight against 1 liter of dialysis buffer (10 mM MOPS, pH 6.5, 50 mM NaCl, 0.1 mM EGTA, 0.02% Brij 35 and 1 mM PMSF). In the morning, a fine white precipitate was removed by centrifugation and the resulting supernatant was loaded onto an 8 ml (8×75 mm) MonoS high performance liquid chromatography column (Pharmacia Biotechnology, Inc., Piscataway, N.J., USA) equilibrated in buffer B (10 mM MOPS, pH 6.5, 0.1 mM EGTA) containing 50 mM NaCl. The column was washed with 10 bed volumes of buffer B containing 50 mM NaCl, and developed with a 50 ml linear gradient of increasing NaCl (50 to 500 mM). Recombinant protein 7116626 eluted as a sharp peak at 300 mM NaCl.

2. Purification of Insoluble Proteins from Inclusion Bodies

The following steps were carried out at 4° C. Cell pellets were resuspended in lysis buffer with 10% glycerol 200 μg/ml lysozyme, 5 mM EDTA, 1 mM PMSF and 0.1%-mercaptoethanol. After passage through the cell disrupter, the resulting homogenate was made 0.2% deoxycholate, stirred 10 minutes, then centrifuged at 20,000×g, for 30 min. The pellets were washed with lysis buffer containing 10% glycerol, 10 mM EDTA, 1% Triton X-100, 1 mM PMSF and 0.1%-mercaptoethanol, followed by several washes with lysis buffer containing 1 M urea, 1 mM PMSF and 0.1% 2-mercaptoethanol. The resulting white pellet was composed primarily of inclusion bodies, free of unbroken cells and membranous materials.

Recombinant Proteins 26054702 (SEQ ID NO: 9450), 16225006 (SEQ ID NO: 9642), 30100332 (SEQ ID NO: 5933), 4721061 (SEQ ID NO: 5181), 978977 (SEQ ID NO: 523)

The following steps were carried out at room temperature. Purified inclusion bodies were dissolved in 20 ml 8.0 M urea in lysis buffer with 1 mM PMSF and 0.1% 2-mercaptoethanol, and incubated at room temperature for 1 hour. Materials that did not dissolve were removed by centrifugation. The clear supernatant was filtered, then loaded onto a Ni²⁺-NTA agarose column pre-equilibrated in 8.0 M urea in Lysis Buffer. The column was washed with 250 ml (50 bed volumes) of lysis buffer containing 8 M urea, 1.0 mM PMSF and 0.1% 2-mercaptoethanol, and developed with sequential steps of lysis buffer containing 8M urea, 1 mM PMSF, 0.1% 2-mercaptoethanol and 20, 100, 200, and 500 mM imidazole in succession. Fractions were monitored by absorbance at OD₂₈₀ nm, and peak fractions were analyzed by SDS-PAGE. Fractions containing the recombinant protein eluted at 100 mM imidazole.

Recombinant Proteins 29479681 (SEQ ID NO: 5379), 26380318 (SEQ ID NO: 4851)

The pellet containing the inclusion bodies was solubilized in buffer B containing 8 M urea, 1 mM PMSF and 0.1% 2-mercaptoethanol, and incubated for 1 hour at room temperature. Insoluble materials were removed by centrifugation at 20,000×g for 30 min, and the cleared supernatant was loaded onto a 15 ml (1.6×7.5 cm) SP-Sepharose column pre-equilibrated in buffer B, 6 M urea, 1 mM PMSF, 0.1% 2-mercaptoethanol. After washing the column with 10 bed volumes, the column was developed with a linear gradient from 0 to 500 mM NaCl.

Dialysis and Concentration of Protein Samples

Urea was removed slowly from the protein samples by dialysis against Tris-buffered saline (TBS; 10 mM Tris pH 8.0, 150 mM NaCl) containing 0.5% deoxycholate (DOC) with sequential reduction in urea concentration as follows; 6M, 4M, 3M; 2M, 1M, 0.5 M and finally TBS without any urea. Each dialysis step was conducted for a minimum of 4 hours at room temperature.

After dialysis, samples were concentrated by pressure filtration using Amicon stirred-cells. Protein concentrations were measured using the methods of Perkins (1986 Eur. J. Biochem. 157, 169-180), Bradford ((1976) Anal. Biochem. 72, 248-254) and Lowry ((1951) J. Biol. Chem. 193, pages 265-275).

The recombinant proteins purified by the methods described above are summarized in Table 4 below.

TABLE 4

| | | | | | | | |
|---|---|---|---|---|---|---|---|
| | | | Bacterial cell | | Relative | Final | |
| | | Gene | fraction used | | MW on | concentra- | |
| J99 | Homolog | symbol | to purify | | SDS- | tion of | Compo- |
| Sequence | identified | of | recombinant | Method of | PAGE | purified | sition of |
| Identifier | by Blast | Homolog | proteins | purification | gel | protein | buffer |

Outer Membrane Proteins

| | | | | | | | |
|---|---|---|---|---|---|---|---|
| 16225006 | P28635 | YEAC | Inclusion | His-Tag | 18 kDa | 5 mg/ml | B |
| (SEQ ID | | | bodies | | | | |
| NO: 9642) | | | | | | | |
| 26054702 | P15929 | flgH | Inclusion | His-Tag | 37 kDa | 1.18 | B |
| (SEQ ID | | | bodies | | | mg/ml | |
| NO: 9450) | | | | | | | |
| | | | | | | — | as dry |
| | | | | | | | pellet |
| 7116626 | P26093 | e(P4) | Soluble | His-Tag | 29 kDa | 0.8 mg/ml | A |
| (SEQ ID | | | fraction | | | | |
| NO: 9048) | | | | | | | |
| | | | | | | 1.85 | C |
| | | | | | | mg/ml | |
| 29479681 | P13036 | fecA | Inclusions | SP- | 23 kDa | 2.36 | B |
| (SEQ ID | | | bodies | Sepharose | | mg/ml | |
| NO: 5379) | | | | | | | |
| | | | | | | 0.5 mg/ml | B |
| | | | | | | — | as dry |
| | | | | | | | pellet |
| 14640637 | P16665 | TPF1 | Soluble | His-Tag | 17 kDa | 2.4 mg/ml | A |
| (SEQ ID | | | fraction | | | | |
| NO: 8015) | | | | | | | |

gel filtration S100 HR

Periplasmic/Secreted Protein

| | | | | | | | |
|---|---|---|---|---|---|---|---|
| 30100332 | P23847 | dppA | Inclusion | His-Tag | 11 kDa | 2.88 | B |
| (SEQ ID | | | bodies | | | mg/ml | |
| NO: 5933) | | | | | | | |
| 4721061 | P36175 | GCP | Inclusion | His-Tag | 38 kDa | 2.8 mg/ml | B |
| (SEQ ID | | | bodies | | | | |
| NO: 5181) | | | | | | | |

Other Surface Proteins

| | | | | | | | |
|---|---|---|---|---|---|---|---|
| 4821082 | P08089 | M | Inclusion | His-Tag | 20 kDa | 1.16 | B |
| (SEQ ID | | protein | bodies | | | mg/ml | |
| NO: 9730) | | | | | | | |
| 978477 | L28919 | FBP54 | Inclusion | SP- | 44 kDa | 2.56 | B |
| (SEQ ID | | | bodies | Sepharose | | mg/ml | |
| NO: 5231) | | | | | | | |
| | | | | | | 0.3 mg/ml | B |

Inner Membrane Proteins

| | | | | | | | |
|---|---|---|---|---|---|---|---|
| 26380318 | P15933 | fliG | Inclusion | SP- | 11 kDa | 22 mg/ml | B |
| (SEQ ID | | | bodies | Sepharose | | | |
| NO: 4851) | | | | | | | |

Control Proteins with His-Tag

| | P00722 | lacZ | Soluble | His-Tag | 116 kDa | 10 mg/ml | A |
|---|---|---|---|---|---|---|---|
| | | | fraction | | | | |
| | | | | gel filtration S200 HR | | | |
| ppiB | | ppiB | Soluble | His-Tag | 21 kDa | 4.4 mg/ml | A |
| (SEQ ID | | | fraction | | | | |
| NO: 7674) | | | | | | | |

gel filtration S100 HR

Buffer compositions:
A = 10 mM Hepes pH 7.5, 150 mM NaCl, 0.1 mM EGTA
B = 10 mM Tris pH 8.0, 150 mM NaCl, 0.5% DOC
C = 10 mM MOPS pH 6.5, 300 mM NaCl, 0.1 EGTA

IV. Analysis of H. pylori Proteins as Vaccine Candidates

To analyze H. pylori proteins for use in the vaccine formulations of the invention, several H. pylori proteins were expressed, characterized immunologically and tested in animal efficacy studies as outlined below. Specifically, the immunomodulatory effects of H. pylori proteins were investigated in a mouse/H. pylori model which mimics the human H. pylori infection in humans. In these studies, the effect of oral immunization of selected H. pylori polypeptides in H. pylori infected mice was determined. Those skilled in the art will recognize that in the examples outlined below, the use of cholera toxin (CT) was only illustrative and is not essential for achieving oral immunization.

Identification, Cloning and Expression of Recombinant Helicobacter Pylori Sequences.

To facilitate the cloning, expression and purification of membrane and/or secreted proteins from H. pylori, the pET gene expression system (Novagen), for cloning and expression of recombinant proteins in Escherichia coli was selected. Further, for proteins that have a signal sequence at their amino-terminal end, a DNA sequence encoding a peptide tag (His-tag) was fused to the 5′ end of the H. pylori DNA sequences of interest in order to facilitate purification of the recombinant protein products.

PCR Amplification and Cloning of DNA Sequences Containing ORFs for Membrane and Secreted Proteins from the J99 Strain of Helicobacter Pylori.

The sequences selected (from the list of the DNA sequences of the invention) for cloning from H. pylori strain J99 were prepared for amplification cloning by the polymerase chain reaction (PCR). All of the selected sequences encode for outer membrane H. pylori proteins, with vac9 (SEQ ID NO:4974), vac10 (SEQ ID NO:5016), vac22 (SEQ ID NO:4967) and vac41 (SEQ ID NO:5051) sequences all sharing a terminal phenylalanine residue. Likewise, the vac32 (SEQ ID NO:5091), vac36 (SEQ ID NO:5146) and vac37 (SEQ ID NO:5139) sequences all share a terminal phenylalanine residue and a tyrosine cluster at the C-terminus. Synthetic oligonucleotide primers for each ORF of interest (Table 5) specific for the predicted mature 5′ end of the ORF and downstream (3′) of the predicted translational termination codon were designed and purchased (GibcoBRL Life Technologies, Gaithersburg, Md., USA). All forward primers (specific for the 5′ terminus of the region of ORF of interest) were designed to include a BamIII restriction site followed by a NdeI restriction site. These primers were designed to permit the initiation of protein translation at a methionine residue encoding within the NdeI restriction site sequence (in the case of producing a non His-tagged recombinant protein) or to fuse in frame with the DNA sequence encoding the His-tag (for producing His-tagged recombinant protein), followed by the coding sequence for the remainder of the native H. pylori DNA. All reverse oligonucleotide primers (specific for downstream (3′) of the predicted translational termination codon of the ORF) were designed to include an EcoRI restriction site at the 5′ terminus. This combination of primers would enable each ORF of interest to be cloned into pET28b (to produce a His-tagged recombinant protein) or pET30a (to produce a non His-tagged or native recombinant protein). The pET28b vector provides sequence encoding an additional 20 amino-terminal amino acids (plus the methionine in the NdeI restriction site) including a stretch of six histidine residues which makes up the His-tag.

Genomic DNA prepared from H. pylori strain J99 (ATCC 55679) was used as the source of template DNA for the PCR amplification reactions (Current Protocols in Molecular Biology, John Wiley and Sons, Inc., F. Ausubel et al., eds., 1994). To amplify a DNA sequence containing a specific H. pylori ORF, genomic DNA (50 nanograms) was introduced into a reaction tube containing 200 nanograms of both the forward and reverse synthetic oligonucleotide primer specific for the ORF of interest, and 45 microliters of PCR SuperMix purchased (GibcoBRL Life Technologies, Gaithersburg, Md., USA) in a total of 50 microliters. The PCR SuperMix is supplied in 1.1× concentrations and contains 22 mM Tris-HCl (pH 8.4), 55 mM KCl, 1.65 mM MgCl₂, 220 micromolar of each dATP, dCTP, dGTP and dTTP, 22 units recombinant Taq polymerase/ml and stabilizers. The following thermal cycling conditions were used to obtain amplified DNA products for each ORF using a Perkin Elmer Cetus/Gene Amp PCR System thermal cycler.

TABLE 5

Oligonucleotide primers

| | | |
|---|---|---|
| Gene | Forward primer | Reverse primer |

| | | |
|---|---|---|
| vac9 | CGCGGATCCATATGGCTGAAA | CCGGAATTCATCAGTATTCAA |
| (nt SEQ ID | AAACGCCTTTTTTTAAAACTAA | TGGGAATAAAGCC (SEQ ID |
| NO:212) | AAACCAC (SEQ ID NO: 9771) | NO: 9772) |
| (aa SEQ ID NO: 4974) | | |
| vac 10 | CGCGGATCCATATGAAAGAAG | CCGGAATTCGCTTAAAAGAAA |
| (nt SEQ ID | AAGAAAAAGAAGAAAAAAAG | ATAGTCCCCCAAACGC (SEQ |
| NO:254) | ACAGAAAGG (SEQ ID NO: 9773) | ID NO: 9774) |
| (aa SEQ ID NO: 5016) | | |
| vac22 | CGCCGGATCCATATGAAAGAG | CCGGAATTCATATAAATATCA |
| (nt SEQ ID | GTCATTCCACCCCTTCAACCCC | TATAGGCAGAAAAAC (SEQ ID |
| NO:205) | (SEQ ID NO: 9775) | NO: 9776) |
| (aa SEQ ID NO: 4967) | | |
| vac32 | CGCGGATCCATATGGAGGCAG | CCGGAATTCGATTGATTTTGTC |
| (ft SEQ ID | AGCTTGATGAAAAATC (SEQ ID | AAATCTAAAATCCC (SEQ ID |
| NO:329) | NO: 9777) | NO: 9778) |
| (aa SEQ ID NO:5091) | | |
| vac36 (hop | TATTATACATATGGAAGAAGA | TAATCTCGAGTTTAGAAGGCG |
| B) | TGGG (SEQ ID NO: 9779) | TA (SEQ ID NO: 9780) |
| (nt SEQ ID | | |
| NO:384) | | |
| (aa SEQ ID NO:5146) | | |
| vac37 | TTATATTCATATGGAAGACGAT | AATTCTCGAGCCTCTTTATAA |
| (i-hop) | GGC (SEQ ID NO: 9781) | GCC (SEQ ID NO:9782) |
| (nt SEQ ID | | |
| NO:377) | | |
| (aa SEQ ID NO: 5139) | | |
| vac41 | CGCGGATCCATATGGTAGAAG | CCGGAATTCGGAGCCAATAGG |
| (nt SEQ ID | CCTTTCAAAAACACCAAAAAG | GAGCTAAAGCC (SEQ ID NO: |
| NO:289) | ACGG (SEQ ID NO: 9783) | 9784) |
| (aa SEQ ID NO: 5051) | | |

Sequences for Vac32 (SEQ ID NO: 5091), Vac9 (SEQ ID NO: 4974) and Vac22 (SEQ ID NO: 4967)

Denaturation at 94° C. for 30 sec

35 cycles at 94° C. for 15 sec, 55° C. for 15 sec, and 72° C. for 1.5 min

Reactions were concluded at 72° C. for 8 minutes

Sequences for Vac10 (SEQ ID NO: 5016) and Vac41 (SEQ ID NO: 5051)

Denaturation at 94° C. for 30 sec

35 cycles at 94° C. for 15 sec, 55° C. for 15 sec, and 72° C. for 2.5 min

Reactions were concluded at 72° C. for 8 minutes

Sequences for Vac36 (SEQ ID NO: 5146) and Vac37 (SEQ ID NO: 5139)

Denaturation at

2 cycles at 94° C. for 15 sec, 30° C. for 15 sec, and 72° C. for 1.5 min

23 cycles at 94° C. for 15 sec, 55° C. for 1.5 sec, and 72° C. for 1.5 min

Reactions were concluded at 72° C. for 6 minutes

Upon completion of the thermal cycling reactions, each sample of amplified DNA was subjected to electrophoresis on 1.0% agarose gels. The DNA was visualized by exposure to ethidium bromide and long wave UV irradiation, and cut out in gel slices. DNA was purified using the Wizard PCR Preps Kit (Promega Corp., Madison, Wis., USA), and then subjected to digestion with BamHI and EcoRI (Current Protocols in Molecular Biology, John Wiley and Sons, Inc., F. Ausubel et al., eds., 1994). The digested PCR amplicon was then re-electrophoresed and purified as before.

Ligation of H. Pylori DNA Sequences into Cloning Vectors

The pOK12 vector (J. Vieira and J. Messing, Gene 100:189-194, 1991) was prepared for cloning for digestion with BamHI and EcoRI in the case of Vac9, 10, 22, 31 and 32, whereas the pSU21 vector (B. Bartolome et al., Gene 102:75-78, 1991) was prepared for cloning by digestion with BamHI and EcoRI in the case of Vac 41 (Current Protocols in Molecular Biology, John Wiley and Sons, Inc., F. Ausubel et al., eds., 1994). The vectors were subjected to electrophoresis on 1.0% agarose gels and purified using the Wizard PCR Preps kit (Promega Corp., Madison, Wis., USA). Following ligation of the purified, digested vector and the purified, digested amplified H. pylori ORF, the products of the ligation reaction were transformed into E. coli JM 109 competent cells according to standard methods (Current Protocols in Molecular Biology, John Wiley and Sons, Inc., F. Ausubel et al., eds., 1994). Individual bacterial colonies were screened for those containing the correct recombinant plasmids by incubating in LB broth overnight (plus 25 ug/ml kanamycin sulfate for the pOK12 based plasmids or 25 ug/ml chloramphenicol for the pSU21 based plasmids) followed by plasmid DNA preparation using the Magic Minipreps system (Promega Corp., Madison, Wis., USA), and then analyzed by restriction digestion (Current Protocols in Molecular Biology, John Wiley and Sons, Inc., F. Ausubel et al., eds., 1994).

Cloning of H. Pylori DNA Sequences into the pET28b and pET30a Prokaryotic Expression Vectors

Both the pET28b and pET30a expression vectors were prepared for cloning by digestion with NdeI and EcoRI (Current Protocols in Molecular Biology, John Wiley and Sons, Inc., F. Ausubel et al., eds., 1994). The H. pylori DNA sequences were removed from pOK12 (Vac9, 10, 23, 31 and 32) or pSU21 (Vac41) plasmid backbones by digestion with NdeI and EcoRI (Current Protocols in Molecular Biology, John Wiley and Son, Inc., F. Ausubel et al., eds., 1994). The pET28b, pET30a and H. pylori DNA sequences were all electrophoresed on a 1% agarose gel and purified using the Wizard PCR Preps kit (Promega Corp., Madison Wis., USA). Following ligation of the purified, digested expression vector and the purified, digest H. pylori DNA sequences, the products of the ligation reaction were transformed into E coli JM109 competent cells (Current Protocols in Molecular Biology, John Wiley and Sons, Inc., F. Ausubel et al., eds., 1994). Individual bacterial colonies were screened for those containing the correct recombinant plasmids by preparing plasmid DNA as described above followed by analysis by restriction digestion profiles and DNA sequencing (Current Protocols in Molecular Biology, John Wiley and Sons, Inc., F. Ausubel et al, eds., 1994). These recombinant plasmids were then used to transform specific E. coli expression strains.

Transformation of Competent Bacteria with Recombinant Expression Plasmids

Competent bacterial strains (BL21 (DE3), BL21 (DE3)pLyS, HMS174(DE3) and HMS174(DE3)pLysS were prepared and transformed with the recombinant pET28b expression plasmids carrying the cloned H. pylori sequences according to standard methods (Current Protocols in Molecular Biology, John Wiley and Sons, Inc., F. Ausubel et al., eds., 1994). These expression host strains contain a chromosomal copy of the gene for T7 RNA polymerase. These hosts are lysogens of bacteriophage DE3, a lambda derivate that carries the lacI gene, the lacUV5 promoter and the gene for T7 RNA polymerase. T7 RNA polymerase expression is induced by the addition of isopropyl-β-D thiogalactoside (1PTG), and the T7 RNA polymerase then transcribes any target plasmid, such as pET28b, that carries a T7 promoter sequence and a gene of interest.

Expression of Recombinant H. Pylori Sequences in E. Coli

Transformants were collected from LB agar plates containing 25 ug/ml kanamycin sulfate (ensures maintenance of the pET28b-based recombinant plasmids) and used to inoculate LB broth containing 25 ug/ml kanamycin sulfate and grown to an optical density at 600 nm of 0.5 to 1.0 OD units, at which point 1 mM 1 PTG was added to the culture for one to three hours to induce gene expression of the H. pylori recombinant DNA constructions. After induction of gene expression with 1PTG, bacteria were pelleted by centrifugation and resuspended in SDS-PAGE solubilization buffer and subjected to SDS-PAGE (Current Protocols in Molecular Biology, John Wiley and Sons, Inc., F. Ausubel et al., eds., 1994). Proteins were visualized by staining with Coomassie Brilliant Blue or detected by western immunoblotting using the specific anti-His tag monoclonal antibody (Clontech, Palo Alto, Calif., USA) using standard methods (Current Protocols in Molecular Biology, John Wiley and Sons, Inc., F. Ausubel et al., eds., 1994). The host strain that provided the highest level of recombinant protein production was then chosen for use in a large-scale induction in order to purify the recombinant protein. All of the following proteins listed were expressed recombinantly and the strain giving the highest level of expression listed: BL21(DE3) (vac31, vac26, vac37; BL21(DE3) pLysS (vac 9, 32); HMS174(DE3) (vac10).

Purification of Recombinant Proteins and Generation of Specific Antiserum

Large scale cultures were inoculated and grown as above, and induced with 1 mM 1 PTG for 3 hours. After induction, bacteria were pelleted by centrifugation in a Sorvall centrifuge at 3500×g for 15 min at 4° C. All of the expressed recombinant proteins were present in the insoluble inclusion body fraction. Inclusion bodies were purified according to standard protocols (Antibodies, Cold Spring Harbor Laboratory Press, E. Harlow and D. Lane, eds., 1988). The recombinant protein produced by vac32 was solubilized in 8M urea and partially purified by nickel chromatography (REF here). Denatured recombinant proteins were purified by electrophoresis on SDS-PAGE gels, and after visualization with Coomassie Brilliant Blue, the protein was excised from the gel and the gel slices homogenized. This material was used to raise specific polyclonal antibodies in mice or rabbits according to standard protocols (Antibodies, Cold Spring Harbor Laboratory Press, E. Harlow and D. Lane, eds., 1988).

Immunological Characterization of Recombinant Proteins

In all cases where antibody was attempted to be raised, high titre antisera was generated, confirming the immunogenicity of the recombinant proteins. Further, these specific antisera were used to analyze whether the protein encoded by the cloned gene was expressed in H. pylori. Western immunoblot analysis using standard protocols (Current Protocols in Molecular Biology, John Wiley and Sons, Inc., F. Ausubel et al., eds., 1994) confirmed that the H. pylori strain J99 did express proteins of the expected molecular weight that reacted with the vac10, vac32, vac31, vac36 antiserum. The specific antiserum was also used to determine the level of antigenic conservation between a large number of H. pylori isolates that had been obtained from distinct geographical sites around the world, and from all types of clinical manifestations, including gastritis, duodenal ulcer, gastric ulcer and gastric cancer. It was found that every strain produced a protein that reacted specifically with each antiserum.

Further, H. pylori cells from strains J99, 17874, AH244 and SS1 were fractionated into different cellular compartments (Doig and Trust 1994 Infect. Immun. 62:4526-4533: O'Toole et al. 1995 J. Bacteriol. 177:6049-6057). The specific antiserum was used to probe these fractions by western immunoblot to identify in which fraction the protein was localized. In all cases, the immunoreactive protein was present in the outer membrane as had been predicted by the sequence features and motif searches described herein.

Demonstration of Protein Efficacy as a Vaccine

Purification of vac36 (SEQ ID NO: 5146) for Efficacy Studies

All the following steps were carried out at 4° C. Cell pellets were resuspended in 5 volumes per gram of cell of lysis buffer (50 mM Sodium Phosphate pH 8.0, 0.5 M NaCl, 5 mM Imidazole) with 10 mM EDTA, 1 mM phenylmethylsulfonyl fluoride (PMSF) and 0.1% β-mercaptoethanol, and ruptured by several passages through a small volume microfluidizer (Model M-110S, Microfluidics International Corporation, Newton, Mass.). The resulting homogenate was made 0.2% sodium deoxycholate (DOC), stirred 20 minutes, then centrifuged (10,000 g×30 nm). The pellets were washed twice with Lysis Buffer containing 10 mM EDTA, 1% Triton X-100, 1 mM PMSF and 0.1% β-mercaptoethanol, then with lysis buffer containing 1 M urea, 1 mM PMSF and 0.1% β-mercaptoethanol. The resulting white pellet is composed primarily of inclusion bodies, free of unbroken cells and membranous materials.

The inclusion bodies were dissolved in 20 ml 6M guanidine-HCl in lysis buffer with 1 mM PMSF and 0.1% β-mercaptoethanol, and incubated on ice for 1 hour. Materials that did not dissolve were removed by centrifugation (100,000 g×30 min.) The clear supernatant was filtered through a 0.8 μm Supor filter (Gelman Sciences, FRG) and then load directly onto a 10 ml Ni²⁺-NTA agarose column (Hochuli et al. 1987) pre-equilibrated in 6M guanidine-HCl in Lysis Buffer containing 1 mM PMSF and 0.1% β-Mercaptoethanol. The column was washed with 20 ml (2 bed volumes) of Lysis Buffer containing 6M guanidine-HCl, 1 mM PMSF and 0.1% β-mercaptoethanol, then guanidine-HCl was removed slowly with a 100 ml linear gradient (from 6M to 0 M Guanidine-HCl) of lysis buffer containing 0.5% Brij 35, 1 mM PMSF, 0.1% β-mercaptochanol. Next, the column was developed with a 25 ml linear gradient of increasing imidazole (5 to 500 mM) in Lysis buffer containing 0.5% Brij 35, 1 mM PMSF and 0.1% β-mercaptoethanol. The recombinant proteins elute as a peak centered at 100 mM imidazole.

Fractions containing the recombinant proteins were pooled and then concentrated to approximately 8 ml by centrifugal filtration (Centriprep-10, Amicon, Mass.), and loaded directly onto a 350-ml column (2.2×91 cm) of Sephacyl S-100 HR gel filtration medium equilibrated in Buffer A (50 mM Sodium Phosphate, pH 8.0, 500 mM NaCl, 0.1 mM EGTA, 1 mM PMSF, 0.1%β-mercaptoethanol, 0.5% Brij 35) and ran in Buffer A at 30 ml/h. Fractions containing the recombinant protein were identified by absorbance at 280 nm and analyzed by SDS-PAGE. Fractions were pooled, concentrated to 1.5 to 2 mg/ml and dialysed overnight against 10 mM Potassium Phosphate pH 7.5, 150 mM NaCl, 0.1 mM EGTA and 0.5% Brij 35. The concentration of protein in the dialysate was quantified, then aliquoted prior to freezing at −20° C.

Mouse Model of Heliocobacter Pylori Infection

A murine model of H. pylori infection was produced by infection of C57BL/6 mice with with H. pylori Sydney strain SS1 and was used to assess the efficacy of recombinant H. pylori vac36. This mouse-adapted H. pylori strain is cagA+ vacA+, shows colonization levels in C57BL/6 mice equivalent to those observed in humans, forms adhesion pedestals, colonizes for at least 8 months, and elicits a chronic-active gastritis and mucosal atrophy (Lee et al., Gastroenterology, 112:1386-1397, 1997). Dose-response studies have shown 100% infection rates of inbred C57BL/6 and Balb/C mice at 8 weeks post-challenge with a single inoculation of 106 organisms.

Animals

Female SPF BALB/c mice were purchased from Bomholt Breeding center (Denmark). They were kept in ordinary makrolon cages with free supply of water and food. The animals were 4-6 weeks old at arrival.

Infection

After a minimum of one week of acclimatization, the animals were infected with a type 2 strain (VacA negative) of H. pylori (strain 244, originally isolated from an ulcer patient). In our hands, this strain has earlier proven to be a good colonizer of the mouse stomach. The bacteria were grown overnight in Brucella broth supplemented with 10% fetal calf serum, at 37 C in a microaerophilic atmosphere (10% CO₂, 5% O₂). The animals were given an oral dose of omeprazole (400 μmol/kg) and 3-5 h after this an oral inoculation of H. pylori in broth (approximately 10⁸ cfu/animal). Positive take of the infection was checked in some animals 2-3 weeks after the inoculation.

Antigens

Recombinant H. pylori antigens were chosen based on their association with externally exposed H. pylori cell membrane. These antigens were selected from the following groups: (1.) Outer Membrane Proteins; (2.) Periplastic/Secreted proteins; (3.) Outer Surface proteins; and (4.) Inner Membrane proteins. All recombinant proteins were constructed with a hexa-HIS tag for purification reasons and the non-Helicobacter pylori control protein (β-galactosidase from E. coli; LacZ), was constructed in the same way.

All antigens were given in a soluble form, i.e. dissolved in either a HEPES buffer or in a buffer containing 0.5% Deoxycholate (DOC).

The antigens are listed in Table 6 below.

TABLE 6
Helicobacter pylori proteins

Outer membrane Proteins
SEQ ID NO: 8015
SEQ ID NO: 5379
SEQ ID NO: 9048
SEQ ID NO: 5181
SEQ ID NO: 9642
Periplastic/Secreted proteins
SEQ ID NO: 5933
Other cell envelope proteins
SEQ ID NO: 9730
SEQ ID NO: 5231
Flagella-associated proteins
SEQ ID NO: 4851
Control proteins
β-galactosidase (LacZ)

Immunizations

Ten animals in each group were immunized 4 times over a 34 day period (day 1, 15, 25 and 35). Purified antigens in solution or suspension were given at a dose of 100 μg/mouse. As an adjuvant, the animals were also given 10 μg/mouse of Cholera toxin (CT) with each immunization. Omeprazole (400 μmol/kg) was given orally to the animals 3-5 h prior to immunization as a way of protecting the antigens from acid degradation. Infected control animals received HEPES buffer+CT or DOC buffer+CT. Animals were sacrificed 2-4 weeks after final immunization. A general outline of the study is shown in Table 7 below.

TABLE 7
Study outline, therapeutic immunization:
Mice were all infected with H. pylori strain
Ah244 at day 30. Proteins are listed by their SeqID #′

| | Mouse strain | | Dates for |
|---|---|---|---|
| Substance | n = 10 | Dose/mouse | dosing |
| 1. Controls, PBS | Balb/c | 0.3 ml | 0, 14, 24, 34 |
| 2. Cholera toxin, 10 μg | Balb/c | 0.3 ml | 0, 14, 24, 34 |
| 3. Protein 14640637 (SEQ ID NO: 8015), | Balb/c | 0.3 ml | 0 14, 24, 34 |
| 100 μg + CT 10 μg | | | |
| 4. Protein 16225006 (SEQ ID NO: 9642), | Balb/c | 0.3 ml | 0 14, 24, 34 |
| 100 μg + CT 10 μg | | | |
| 5. Protein 26054702 (SEQ ID NO: 9450), | Balb/c | 0.3 ml | 0 14, 24, 34 |
| 100 μg + CT 10 μg | | | |
| 6. Protein 26380318 (SEQ ID NO: 4851), | Balb/c | 0.3 ml | 0 14, 24, 34 |
| 100 μg + CT 10 μg | | | |
| 7. Protein 29479681 (SEQ ID NO: 5379), | Balb/c | 0.3 ml | 0 14, 24, 34 |
| 100 μg + CT 10 μg | | | |
| 8. Protein 30100332 (SEQ ID NO: 5933), | Balb/c | 0.3 ml | 0 14, 24, 34 |
| 100 μg + CT 10 μg | | | |
| 9. Protein 4721061 (SEQ ID NO: 5181), | Balb/c | 0.3 ml | 0 14, 24, 34 |
| 100 μg + CT 10 μg | | | |
| 10. Protein 4821082 (SEQ ID NO: 9730), | Balb/c | 0.3 ml | 0 14, 24, 34 |
| 100 μg + CT 10 μg | | | |
| 11. Protein 978477 (SEQ ID NO: 5231), | Balb/c | 0.3 ml | 0 14, 24, 34 |
| 100 μg + CT 10 μg | | | |
| 12. Protein 7116626 (SEQ ID NO: 9048), | Balb/c | 0.3 ml | 0 14, 24, 34 |
| 100 μg + CT 10 μg | | | |

Analysis of Infection

Mucosal infection: The mice were sacrificed by CO₂ and cervical dislocation. The abdomen was opened and the stomach removed. After cutting the stomach along the greater curvature, it was rinsed in saline. The mucosa from the antrum and corpus of an area of 25 mm² was scraped separately with a surgical scalpel. The mucosa scraping was suspended in Brucella broth and plated onto Blood Skirrow selective plates. The plates were incubated under microaerophilic conditions for 3-5 days and the number of colonies was counted. The identity of H. pylori was ascertained by urease and catalase test and by direct microscopy or Gram staining.

The urease test was performed essentially as follows. The reagent, Urea Agar Base Concentrate, was purchased from DIFCO Laboratories, Detroit, Mich. (Catalog # 0284-61-3). Urea agar base concentrate was diluted 1:10 with water. 1 ml of if the diluted concentrate was mixed with 100-200 μl of actively growing H. pylori cells. Color change to magenta indicated that cells were urease positive.

The catalase test was performed essentially as follows. The reagent, N,N,N′,N′-Tetramethyl-p-Phenylenediamine, was purchased from Sigma, St. Louis, Mo. (Catalog # T3134). A solution of the regent (1% w/v in water) was prepared. H. pylori cells were swabbed onto Whatman filter paper and overlaid with the 1% solution. Color change to dark blue indicated that the cells were catalase positive.

Serum antibodies: From all mice serum was prepared from blood drawn by heart puncture. Serum antibodies were identified by regular ELISA techniques, where the specific antigens of Helicobacter pylori were plated.

Mucosal antibodies: Gentle scrapings of a defined part of the corpus and of 4 cm of duodenum were performed in 50% of the mice in order to detect the presence of antibodies in the mucous. The antibody titers were determined by regular ELISA technique as for serum antibodies.

Statistical analysis: Wilcoxon-Mann-Whitney sign rank test was used for determination of significant effects of the antigens on Helicobacter pylori colonization. P<0.05 was considered significant. Because the antrum is the major colonization site for Helicobacter most emphasis was put upon changes in the antral colonization.

Assessment of Gastric H. Pylori Infection

The presence of H. pyloriorganisms in gastric tissue was determined by culture of gastric tissue and by a quantitative urease assay. In the latter method, a longitudinal segment of antrum, representing approximately ¼ of the total antral region was placed in 1 ml of urea broth. After 4 hr, the extent of color change resulting from urea hydrolysis and increased pH was quantiated by spectrophotometric measurement of A₅₅₀ (Fox et al., Immunol. 88:400-406, 1996). The assay sensitivity is ˜10³ H. pylori organisms. A positive (H. pylori-infected) gastric tissue was defined as that sample showing 2 standard deviations above the mean A₅₅₀ value derived from a group of unchallenged uninfected age-matched control mice.

Assessment of Local Immune Response to Immunization in Gastric Tissue

Longitudinal sections of gastric tissues from the esophageal to the duodenal junction were embedded in OCT embedding compound, frozen in liquid nitrogen, and cryosections immunostained with monoclonal antibodies recognizing CD4⁺ or CD8⁺T cells or with antisera against mouse IgA for identification of IgA containing (IgACC) plasma cells (Pappo et al., Infect. Immun. 63:1246-1252, 1995). The degree of local gastric immune response was expressed quantitatively as the number of CD4⁺, CD8⁺ or IgACC cells per mm² of gastric region examined.

Results

Antibodies in sera: All antigens tested given together with CT gave rise to a measurable specific titer in serum. The highest responses were seen with SEQ ID NOs:865, 812, 658, 447, and 820 (see FIG. 1).

Antibodies in mucus: In the mucus scrapings, specific antibodies against all antigens tested were seen. By far the strongest response was seen with SEQ ID NOs:685, followed by 447, 865, and 658 (see FIG. 2).

Therapeutic immunization effects:

All control animals (BALB/c mice) were well colonized with H. pylori (strain AH244) in both antrum and corpus of the stomach. Of the antigens tested 3 proteins (SEQ ID NOs: 812, 820, and 447) gave a good and significant reduction and/or eradication of the H. pylori infection. The degree of colonization of the antrum was lower following immunization with SEQ ID NOs: 880, 658, and 865 compared to control. The effect of SEQ ID NOs:465, 677, and 685 did not differ from control. The control protein lacZ, i.e. the non-H. pylori protein, had no eradication effect and in fact had higher Helicobacter colonization compared to the HEPES+CT control. All data are shown in FIGS. 3 and 4 for proteins dissolved in HEPES and DOC respectively. Data is shown as geometric mean values. n=8-10 Wilcoxon-Mann-Whitney sign rank test *=p<0.05;×/10=number of mice showing eradication of H. pylori over the total number of mice examined.

TABLE 8
Recombinant vac36 antigen protects
mice from challenge with H. pylori

Vaccine

| Treatment | Urease | | H. pylori | |
|---|---|---|---|---|
| Group | Activity^{a} | p^{b} | burden^{c} | p^{b} |
| vac36 (SEQ ID | 0.199 ± 0.080 | 0.0022 | 55,800 ± 12,599 | 0.0125 |
| NO: 5146) | | | | |
| H. pylori lysate | 0.057 ± 0.007 | 0.0002 | 2,360 ± 955 | 0.0002 |
| buffer | 1.655 ± 0.420 | — | 131,000 ± 18,391 | — |

^{a}Urease activity is expressed as mean A₅₅₀ ± SEM of duplicate antral samples from n = 10 mice/group.
^{b}by Wilcoxon Rank Sum Test compared with mice immunized with CT adjuvant alone
^{c}The level of H. pylori in gastric tissue was assessed by bacterial counts, and shown as mean colony forming units ± SEM

The data presented indicate that all of the H. pylori associated proteins included in this study, when used as oral immunogens in conjunction with the oral adjuvant CT, resulted in stimulation of an immune response as measured by specific serum and mucosal antibodies. A majority of the proteins led to a reduction, and in some cases complete clearance of the colonization of H. pylori in this animal model. It should be noted that the reduction or clearance was due to heterologous protection rather than homologous protection (the polypeptides were based on the H. pylori J99 strain sequence and used in the therapeutic immunization studies against a different (AH244) challenge strain), indicating the vaccine potential against a wide variety of H. pylori strains.

The highest colonization in the antrum was seen in animals treated with the non-Helicobacter protein LacZ, indicating that the effects seen with the Helicobacter pylori antigens were specific.

Taken together these data strongly support the use of these H. pylori proteins in a pharmaceutical formulation for the use in humans to treat and/or prevent H. pylori infections.

Protective activity of purified recombinant H. pylori vac36 (SEQ ID NO: 5146) antigen The ability of purified recombinant vac36 (SEQ ID NO: 5146) antigen derived from H. pylori to interfere with the establishment of an H. pylori infection was examined in mice. Groups (n=10) of 6-8 week-old female C57BL/6 mice were immunized orally 4 times at weekly intervals as follows: 1)100 μg of recombinant vac36 (SEQ ID NO: 5146) antigen and 10 μg cholera toxin (CT) adjuvant, 2) 1 mg H. pylori lysate antigens and 10 μg CT, and 3) 0.2 M bicarbonate buffer and 10 ug CT adjuvant. The mice were challenged 2 weeks later on 3 consecutive days by oral administration of 10⁸ H. pylori organisms. The experiment was terminated 2 weeks post-challenge, and the H. pylori infection level assessed by bacterial colony counts and by quantitative urease assays.

Oral immunization with vac36 (SEQ ID NO: 5146) antigen interfered with the establishment of H. pylori infection upon challenge with live H. pylori organisms. Mice immunized with purified recombinant vac36 (SEQ ID NO: 5146) antigen exhibited a significantly lower level of colonization by H. pylori, as assessed by gastric urease activity and bacterial count assays (Table 8). Oral immunization with vac36 (SEQ ID NO: 5146) antigen also resulted in the generation of a local protective gastric immune response. Greater numbers of CD4⁺T cells and of IgACC were recruited in the gastric tissues of vac36-immunized mice when compared with unimmunized H. pylori-infected mice (Table 9).

TABLE 9
vac36-immunized mice generate a local gastric immune response upon challenge with
H. pylori

Vaccine

| Treatment | CD4+ | | | CD8+ | | | IgACC | | |
|---|---|---|---|---|---|---|---|---|---|
| Group | cardia^{a} | corpus | antrum | cardia | corpus | antrum | cardia | corpus | antrum |
| vac36 | 33 ± 9^{a} | 54 ± 8* | 31 ± 8 | 3 ± 2 | 0 | 1 ± 1 | 24 ± 12 | 79 ± 16 | 67 ± 13 |
| (SEQ | | | | | | | | | |
| ID NO: | | | | | | | | | |
| 5146) | | | | | | | | | |
| H. | 31 ± 13 | 36 ± 19 | 24 ± 8 | 4 ± 2 | 2 ± 1 | 2 ± 1 | 31 ± 9 | 73 ± 13* | 79 ± 15 |
| pylori | | | | | | | | | |
| lysate | | | | | | | | | |
| buffer | 12 ± 2 | 27 ± 8 | 18 ± 4 | 1 ± 1 | 0 | 0 | 4 ± 2 | 30 ± 13 | 46 ± 14 |

^{a}Mean number of cells/mm² of gastric region ± SEM
*p < 0.05 by Wilcoxon Rank Sum Test when compared with unimmunized H. pylori infected mice

V. Sequence Variance Analysis of Genes in Helicobacter Pylori Strains

Four genes were cloned and sequenced from several strains of H. pylori to compare the DNA and deduced amino acid sequences. This information was used to determine the sequence variation between the H. pylori strain, J99, and other H. pylori strains isolated from human patients.

Preparation of Chromosomal DNA.

Cultures of H. pylori strains (as listed in Table 12) were grown in BLBB (1% Tryptone, 1% Peptamin 0.1% Glucose, 0.2% Yeast Extract 0.5% Sodium Chloride, 5% Fetal Bovine Serum) to an OD₆₀₀ of 0.2. Cells were centrifuged in a Sorvall RC-3B at 3500×g at 4° C. for 15 minutes and the pellet resuspended in 0.95 mls of 10 mM Tris-HCl, 0.1 mM EDTA (TE). Lysozyme was added to a final concentration of 1 mg/ml along with, SDS to 1% and RNAse A+T1 to 0.5 mg/ml and 5 units/ml respectively, and incubated at 37° C. for one hour. Proteinase K was then added to a final concentration of 0.4 mg/ml and the sample was incubated at 55 C for more than one hour. NaCl was added to the sample to a concentration of 0.65 M, mixed carefully, and 0.15 ml of 10% CTAB in 0.7M NaCL (final is 1% CTAB/70 mM NaCL) was added followed by incubation at 65° C. for 20 minutes. At this point, the samples were extracted with chloroform:isoamyl alcohol, extracted with phenol, and extracted again with chloroform:isoamyl alcohol. DNA was precipitated with either EtOH (1.5× volumes) or isopropanol (0.6× volumes) at −70° C. for 10 minutes, washed in 70% EtOH and resuspended in TE.

PCR Amplification and Cloning

Genomic DNA prepared from twelve strains of Helicobacter pylori was used as the source of template DNA for PCR amplification reactions (Current Protocols in Molecular Biology, John Wiley and Sons, Inc., F. Ausubel et al., editors, 1994). To amplify a DNA sequence containing an H. pylori ORF, genomic DNA (10 nanograms) was introduced into a reaction vial containing 2 mM MgCl₂, 1 micromolar synthetic oligonucleotide primers (forward and reverse primers, see Table 10) complementary to and flanking a defined H. pylori ORF, 0.2 mM of each deoxynucleotide triphosphate; dATP, dGTP, dCTP, dTTP and 0.5 units of heat stable DNA polymerase (Amplitaq, Roche Molecular Systems, Inc., Branchburg, N.J., USA) in a final volume of 20 microliters in duplicate reactions.

TABLE 10

Oligonucleotide primers used for PCR amplification of H pylon DNA se-
quences.

| Outer membrane | | |
|---|---|---|
| Proteins (SEQ ID NO: | | |
| 9450) | Forward primer 5′ to 3′ | Reverse Primer 5′ to 3′ |
| Protein 26054702 | 5′- | 5′- |
| (SEQ ID NO: 9450) | TTAACCATGGTGAAA | TAGAATTCGCCTCTA |
| (for strains AH4, | AGCGATA-3′ (SEQ ID | AAACTTTAG-3′ (SEQ |
| AH15, AH61, 5294, | NO:9785) | ID NO:9786) |
| 5640, AH18, and | | |
| AH244) | | |
| Protein 26054702 | 5′- | 5′- |
| (SEQ ID NO: 9450) | TTAACCATGGTGAAA | TAGAATTCGCATAAC |
| (for strains AH5, 5155, | AGCGATA-3′ (SEQ ID | GATCAATC-3′ (SEQ ID |
| 7958, AH24,and J99) | NO:9787) | NO:9788) |
| Protein 7116626 (SEQ | 5′- | 5′- |
| ID NO: 9048) | ATATCCATGGTGAGT | ATGAATTCAATTTTT |
| | TTGATGA-3′ (SEQ ID | TATTTTGCCA-3′ (SEQ |
| | NO:9789) | ID NO:9790) |
| Protein 29479681 | 5′- | 5′- |
| (SEQ ID NO: 5379) | AATTCCATGGCTATC | ATGAATTCGCCAAAA |
| | CAAATCCG-3′ (SEQ ID | TCGTAGTATT-3′ (SEQ |
| | NO:9791) | ID NO:9792) |
| Protein 36126938 | 5′- | 5′- |
| (SEQ ID NO: 5122) | GATACCATGGAATTT | TGAATTCGAAAAAGT |
| | ATGAAAAAG-3′ (SEQ | GTAGTTATAC-3′ (SEQ |
| | ID NO:9793) | ID NO:9794) |

The following thermal cycling conditions were used to obtain amplified DNA products for each ORF using a Perkin Elmer Cetus/GeneAmp PCR System 9600 thermal cycler:

Protein 7116626 (SEQ ID NO: 9048) and Protein 36126938 (SEQ ID NO: 5122);

Denaturation at 94° C. for 2 min,

2 cycles at 94° C. for 15 sec, 30° C. for 15 sec and 72° C. for 1.5 min

23 cycles at 94° C. for 15 sec, 55° C. for 15 sec and 72° C. for 1.5 min

Reactions were concluded at 72° C. for 6 minutes.

Protein 26054702 (SEQ ID NO: 9450) for strains AH5, 5155, 7958, AH24, and J99;

Denaturation at 94° C. for 2 min,

2 cycles at 94° C. for 15 sec, 30° C. for 15 sec and 72° C. for 1.5 min

25 cycles at 94° C. for 15 sec, 55° C. for 15 sec and 72° C. for 1.5 min

Reaction was concluded at 72° C. for 6 minutes.

Protein 26054702 (SEQ ID NO: 9450) and Protein 29479681 (SEQ ID NO: 5379) for strains AH4, AH15, AH61, 5294, 5640, AH18, and Hp244;

Denaturation at 94 C for 2 min,

2 cycles at 94° C. for 15 sec, 30° C. for 20 sec and 72° C. for 2 min

25 cycles at 94° C. for 15 sec, 55° C. for 20 sec and 72° C. for 2 min

Reactions were concluded at 72° C. for 8 minutes.

Upon completion of thermal cycling reactions, each pair of samples were combined and used directly for cloning into the pCR cloning vector as described below.

Cloning of H. Pylori DNA Sequences into the pCR TA Cloning Vector.

All amplified inserts were cloned into the pCR 2.1 vector by the method described in the Original TA cloning kit (Invitrogen, San Diego, Calif.). Products of the ligation reaction were then used to transform the TOP10F′ (INVaF′ in the case of H. pylori sequence 350) strain of E. coli as described below.

Transformation of Competent Bacteria with Recombinant Plasmids

Competent bacteria, E coli strain TOP10F′ or E. coli strain INVaF′ were transformed with recombinant pCR expression plasmids carrying the cloned H. pylori sequences according to standard methods (Current Protocols in Molecular Biology, John Wiley and Sons, Inc., F. Ausubel et al., editors, 1994). Briefly, 2 microliters of 0.5 micromolar BME was added to each vial of 50 microliters of competent cells. Subsequently, 2 microliters of ligation reaction was mixed with the competent cells and incubated on ice for 30 minutes. The cells and ligation mixture were then subjected to a “heat shock” at 42° C. for 30 seconds, and were subsequently placed on ice for an additional 2 minutes, after which, samples were incubated in 0.45 milliliters SOC medium (0.5% yeast extract, 2.0% tryptone, 10 mM NaCl, 2.5 mM KCl, 10 mM MgC12, 10 mM MgSO4 and 20, mM glucose) at 37° C. with shaking for 1 hour. Samples were then spread on LB agar plates containing 25 microgram/ml kanamycin sulfate or 100 micrograms/ml ampicillan for growth overnight. Transformed colonies of TOP10F′ or INVaF′ were then picked and analyzed to evaluate cloned inserts as described below.

Identification of Recombinant PCR Plasmids Carrying H. pylori Sequences

Individual TOP10F′ or INVaF′ clones transformed with recombinant pCR-H. pylori ORFs were analyzed by PCR amplification of the cloned inserts using the same forward and reverse primers, specific for each H. pylori sequence, that were used in the original PCR amplification cloning reactions. Successful amplification verified the integration of the H. pylori sequences in the cloning vector (Current Protocols in Molecular Biology, John Wiley and Sons, Inc., F. Ausubel et al., editors, 1994).

Individual clones of recombinant pCR vectors carrying properly cloned H. pylori ORFs were picked for sequence analysis. Sequence analysis was performed on ABI Sequencers using standard protocols (Perkin Elmer) using vector-specific primers (as found in PCRII or pCR2.1, Invitrogen, San Diego, Calif.) and sequencing primers specific to the ORF as listed in Table 11 below.

TABLE 11
OligonucIeotide primers used for sequencing of H vylori DNA sequences.

| Outer membrane | | |
|---|---|---|
| Proteins | Forward primers 5′ to 3′ | Reverse Primers 5′ to 3′ |
| Protein 26054702 | 5′CCCTTCATTTTAGAAA | 5′CTTTGGGTAAAAACGCA |
| (SEQ ID NO: 9450) | TCG-3′ (SEQ ID NO:9795) | TC-3′ (SEQ ID NO:9802) |
| | 5′ATTTCAACCAATTCAA | 5′CGATCTTTGATCCTAAT |
| | TGCG-3′ (SEQ ID | TCA-3′ (SEQ ID NO:9803) |
| | NO:9796) | 5′ATCAAGTTGCCTATGCT |
| | 5′GCCCCTTTTGATTTGA | GA-3′ (SEQ ID NO:9804) |
| | AGCT-3′ (SEQ ID | |
| | NO:9797) | |
| | 5′TCGCTCCAAGATACCA | |
| | AGAAGT-3′ (SEQ ID | |
| | NO:9798) | |
| | 5′CTTGAATTAGGGGCA | |
| | AAGATCG-3′ (SEQ ID | |
| | NO:9799) | |
| | 5′ATGCGTTTTTACCCAA | |
| | AGAAGT-3′ (SEQ ID | |
| | NO:9800) | |
| | 5′ATAACGCCACTTCCTT | |
| | ATTGGT-3′ (SEQ ID | |
| | NO:9801) | |
| Protein 7116626 | 5′TTGAACACTTTTGATT | 5′GTCTTTAGCAAAAATGG |
| (SEQ ID NO: 9048) | ATGCGG-3′ (SEQ ID | CGTC-3′ (SEQ ID NO:9807) |
| | NO:9805) | 5′AATGAGCGTAAGAGAG |
| | 5′GGATTATGCGATTGTT | CCTTC-3′ (SEQ ID NO:9808) |
| | TTACAAG-3′ (SEQ ID | |
| | NO:9806) | |
| Protein | 5′CTTATGGGGGTATTGT | 5′AGGTTGTTGCCTAAAGA |
| 29479681 (SEQ ID NO: 5379) | CA-3′ (SEQ ID NO:9809) | CT-3′ (SEQ ID NO:9811) |
| | 5′AGCATGTGGGTATCC | 5′- |
| | AGC-3′ (SEQ ID NO:9810) | CTGCCTCCACCTTTGATC- |
| | | 3′ (SEQ ID NO:9812) |
| Protein 36126938 | 5′ACCAATATCAATTGGC | 5′CTTGCTTGTCATATCTA |
| (SEQ ID NO: 5122) | ACT-3′ (SEQ ID NO:9813) | GC-3′ (SEQ ID NO:9815) |
| | 5′ACTTGGAAAAGCTCT | 5′- |
| | GCA-3′ (SEQ ID NO:9814) | GTTGAAGTGTTGGTGCTA- |
| | | 3′ (SEQ ID NO:9816) |
| | 5′CAAGCAAGTGGTTTG | 5′GCCCATAATCAAAAAGC |
| | GTTTTAG-3′ (SEQ ID | CCAT-3′ (SEQ ID NO:98 19) |
| | NO:98 17) | 5′CTAAAACCAAACCACTT |
| | 5′TGGAAAGAGCAAATC | GCTTGTC-3′ (SEQ ID |
| | ATTGAAG-3′ (SEQ ID | NO:9820) |
| | NO:9818) | |
| Vector Primers | 5′- | 5′- |
| | GTAAAACGACGGCCAG- | CAGGAAACAGCTATGAC- |
| | 3′ (SEQ ID NO:9821) | 3′ (SEQ ID NO:9822) |

Results

To establish the PCR error rate in these experiments, five individual clones of Protein 26054702 (SEQ ID NO: 9450), prepared from five separate PCR reaction mixtures from H. pylori strain J99, were sequenced over a total length of 897 nucleotides for a cumulative total of 4485 bases of DNA sequence. DNA sequence for the five clones was compared to a DNA sequence obtained previously by a different method, i.e., random shotgun cloning and sequencing. The PCR error rate for the experiments described herein was determined to be 2 base changes out of 4485 bases, which is equivalent to an estimated error rate of less than or equal to 0.04%.

DNA sequence analysis was performed on four different open reading frames identified as genes and amplified by PCR methods from a dozen different strains of the bacterium Helicobacter pylori. The deduced amino acid sequences of three of the four open reading frames that were selected for this study showed statistically significant BLAST homology to defined proteins present in other bacterial species. Those ORFs included: Protein 26054702 (SEQ ID NO: 9450), homologous to the val A & B genes encoding an ABC transporter in F. novicida; Protein 7116626 (SEQ ID NO: 9048), homologous to lipoprotein e (P4) present in the outer membrane of H. influenzae; Protein 29479681 (SEQ ID NO: 5379), homologous to fecA, an outer membrane receptor in iron (III) dicitrate transport in E. coli. Protein 36126938 (SEQ ID NO: 5122) was identified as an unknown open reading frame, because it showed low homology with sequences in the public databases.

To assess the extent of conservation or variance in the ORFs across various strains of H. pylori, changes in DNA sequence and the deduced protein sequence were compared to the DNA and deduced protein sequences found in the J99 strain of H. pylori (see Table 12 below). Results are presented as percent identity to the J99 strain of H. pylori sequenced by random shotgun cloning. To control for any variations in the J99 sequence each of the four open reading frames were cloned and sequenced again from the J99 bacterial strain and that sequence information was compared to the sequence information that had been collected from inserts cloned by random shotgun sequencing of the J99 strain. The data demonstrate that there is variation in the DNA sequence ranging from as little as 0.12% difference (Protein 36126938 (SEQ ID NO: 5122), J99 strain) to approximately 7% change (Protein 26054702 (SEQ ID NO: 9450), strain AH5). The deduced protein sequences show either no variation (Protein 36126938 (SEQ ID NO: 5122), strains AH18 and AH24) or up to as much as 7.66% amino acid changes (Protein 26054702 (SEQ ID NO: 9450), Strain AH5).

TABLE 12
Multiple Strain DNA Sequence analysis of H. pylori Vaccine Candidates

| | | | | | | | | |
|---|---|---|---|---|---|---|---|---|
| J99 Protein #: | 26054702 | 26054702 | 7116626 | 7116626 | 29479681 | 29479681 | 36126938 | 36126938 |
| | (SEQ ID | (SEQ ID | (SEQ ID | (SEQ ID | (SEQ ID | (SEQ ID | (SEQ ID | (SEQ ID |
| | NO: 9450) | NO: 4688) | NO: 9048) | NO: 4286) | NO: 5379) | NO: 617) | NO: 5122) | NO: 360) |

Length of Region

| Sequenced: | 248 a.a. | 746 nt. | 232 a.a. | 696 nt. | 182 a.a. | 548 nt. | 273 a.a. | 819 nt. |
|---|---|---|---|---|---|---|---|---|
| Strain Tested | AA | Nuc. | AA | Nuc. | AA | Nuc. | AA | Nuc. |
| | identity | identity | identity | identity | identity | identity | identity | identity |
| J99 | 100.00% | 100.00% | 100.00% | 100.00% | 100.00% | 100.00% | 99.63% | 99.88% |
| AH244 | 95.16% | 95.04% | n.d. | n.d. | 99.09% | 96.71% | 98.90% | 96.45% |
| AH4 | 95.97% | 95.98% | 97.84% | 95.83% | n.d. | n.d. | 97.80% | 95.73% |
| AH5 | 92.34% | 93.03% | 98.28% | 96.12% | 98.91% | 96.90% | 98.53% | 95.73% |
| AH15 | 95.16% | 94.91% | 97.41% | 95.98% | 99.82% | 97.99% | 99.63% | 96.09% |
| AH61 | n.d. | n.d. | 97.84% | 95.98% | 99.27% | 97.44% | n.d. | n.d. |
| 5155 | n.d. | n.d. | n.d. | n.d. | 99.45% | 97.08% | 98.53% | 95.60% |
| 5294 | 94.35% | 94.37% | 98.28% | 95.40% | 99.64% | 97.26% | 97.07% | 95.48% |
| 7958 | 94.35% | 94.10% | 97.84% | 95.40% | n.d. | n.d. | 99.63% | 96.46% |
| 5640 | 95.16% | 94.37% | 97.41% | 95.69% | 99.09% | 97.63% | 98.53% | 95.48% |
| AH18 | n.d. | n.d. | 98.71% | 95.69% | 99.64% | 97.44% | 100.00% | 95.97% |
| AH24 | 94.75% | 95.04% | 97.84% | 95.40% | 99.27% | 96.71% | 100.00% | 96.46% |

n.d. = not done.

VI. Experimental Knock-Out Protocol for the Determination of Essential H. Pylori Genes as Potential Therapeutic Targets

Therapeutic targets are chosen from genes whose protein products appear to play key roles in essential cell pathways such as cell envelope synthesis, DNA synthesis, transcription, translation, regulation and colonization/virulence.

The protocol for the deletion of portions of H. pylori genes/ORFs and the insertional mutagenesis of a kanamycin-resistance cassette in order to identify genes which are essential to the cell is modified from previously published methods (Labigne-Roussel et al., 1988, J. Bacteriology 170, pp. 1704-1708; Cover et al., 1994, J. Biological Chemistry 269, pp. 10566-10573; Reyrat et al., 1995, Proc. Natl. Acad. Sci. 92, pp 8768-8772). The result is a gene “knock-out.”

Identification and Cloning of H. Pylori Gene Sequences

The sequences of the genes or ORFs (open reading frames) selected as knock-out targets were identified from the H. pylori genomic sequence and used to design primers to specifically amplify the genes/ORFs. All synthetic oligonucleotide primers (Table 13) were designed with the aid of the OLIGO program (National Biosciences, Inc., Plymouth, Minn. 55447, USA), and were purchased from Gibco/BRL Life Technologies (Gaithersburg, Md., USA). Specific primers (F1 and R1) were chosen which flanked most or all of the ORF, depending on its size. If the ORF was smaller than 800 to 1000 base pairs, flanking primers were chosen outside of the open reading frame.

TABLE 13
Oligonucleotide Sequences for Knock-Out Gene/ORFs

| | | | | Deletion- | | | |
|---|---|---|---|---|---|---|---|
| | Acces- | Cloning | | creating | | Targeting | |
| Gene | sion | Primers | | Primers | | Primers | |
| Name | Number | F1 | R1 | F2 | R2 | F3 | R3 |
| rnh | P2332 | TTGCCCCAT | AGAGCGTAT | TCTTGCATC | CGGGTCAAA | AATCCGTTT | AACACTT |
| | 9 | CGTATTGAT | TTCACCCGA | TTAATCCAC | ACGACCACT | CGCTAATTT | ATTTCCT |
| | | AGA (SEQ | AAG | TCC | TAA | AGT | ATA |
| | | ID | (SEQ ID | (SEQ ID | (SEQ ID | (SEQ ID | (SEQ ID |
| | | NO: 9823) | NO: 9824) | NO: 9825) | NO: 9826) | NO: 9827) | NO: 9828 |
| ppi | P2982 | TGGTATAAG | TTGACTAAA | ATAGAGAGC | CCTTTATTG | ATGTCCGTT | TAGGGTG |
| B | 0 | GATTTGAAT | CACATGCGA | GTTGTGTTT | GTTTTGATC | GTCTGTATG | TAGGGAT |
| | | GGA | GAA | AGC | GTG | GAA | GAT |
| | | (SEQ ID | (SEQ ID | (SEQ ID | (SEQ ID | (SEQ ID | (SEQ ID |
| | | NO: 9829) | NO: 9830) | NO: 9831) | NO: 9832) | NO: 9833) | NO: 9834 |
| tsf | P3482 | GCGTTTGGC | GAAATGGAA | GCAAATCCC | GTGGCTAAA | GCTAAAACT | GTTAGGA |
| | 8 | TTCTTCGTT | AATAGCGGT | CAGCCACTT | AATGAGGGC | TCATCGCTC | TTAGAAA |
| | | GTC | CAA | CC | TT | AAT | ATTG |
| | | (SEQ ID | (SEQ ID | (SEQ ID | (SEQ ID | (SEQ ID | (SEQ ID |
| | | NO: 9835) | NO: 9836) | NO: 9837) | NO: 9838) | NO: 9839) | NO: 9840) |
| Mur | P1490 | GTTGGGCAG | CAAACAAAC | CATTGATGC | CGTGGTGGT | CGGGCATTG | TGGTCTATC |
| D | 0 | AAAATAAGG | CTGACAAGA | CTAAAACTT | TTTCCCGTT | TGTTTGTTT | ATGCGAATT |
| | | TGA | AAC | CG | AG | TT | AT |
| | | (SEQ ID | (SEQ ID | (SEQ ID | (SEQ ID | (SEQ ID | (SEQ ID |
| | | NO: 9841) | NO: 9842) | NO: 9843) | NO: 9844) | NO: 9845) | NO: 9846) |
| Mur | P2218 | GCGTTTGGG | CGCGCTAGA | GCCCTGATC | AAGGGCGTT | GGCGTTATT | TTTCACCGG |
| E | 8 | GATTTGATG | GGCTTGTAA | CATTCCCCC | TGTTTCATC | AAGCGACAT | CAATTTTAG |
| | | TTC | AA | CT | TC | CG | CC. |
| | | (SEQ ID | (SEQ ID | (SEQ ID | (SEQ ID | (SEQ ID | (SEQ ID |
| | | NO: 9847) | NO: 9848) | NO: 9849) | NO: 9850) | NO: 9851) | NO: 9852) |
| Alg | P0787 | GCGTTTTGA | GTAAAAACA | GCGTGTTTT | GGAATTTTA | AAATCTCTG | AATCAAAAA |
| A | 4 | TTCTGTCTG | CCGCTAACG | CTAAGGGTT | ACGCTCTTT | TGGGCTTAG | CAAGAGCGT |
| | | TTA | CAT | CA | TT | TG | GG |
| | | (SEQ ID | (SEQ ID | (SEQ ID | (SEQ ID | (SEQ ID | (SEQ ID |
| | | NO: 9853) | NO: 9854) | NO: 9855) | NO: 9856) | NO: 9857) | NO: 9858) |
| met | P1935 | GCCCCAGCC | GGAGGGCGC | TCACGCTTT | CGCTAATCA | TGCCCAAAA | AACGGGTTT |
| L | 8 | CCATAATAC | AATTAAACA | CTAAATCAT | CATCCTTTC | ATCCACTAA | GACACTGAT |
| | | AAA | TCG | CA | TT | CG | GA |
| | | (SEQ ID | (SEQ ID | (SEQ ID | (SEQ ID | (SEQ ID | (SEQ ID |
| | | NO: 9859) | NO: 9860) | NO: 9861) | NO: 9862) | NO: 9863) | NO: 9864) |
| fus | X1627 | GAATGCGGT | GCGTTTTTA | GGGCGATGT | GGATAGCCT | AAGTTTATG | GGAGCAATC |
| A | 8 | GGTTTTAGA | AGACTGAAT | GATTGGCGA | GCCAAAACG | CGGGCGAGA | AGCCATTTT |
| | | GAG | ACA | TT | CC | TT | TC |
| | | SEQ ID | (SEQ ID | (SEQ ID | (SEQ ID | (SEQ ID | (SEQ ID |
| | | NO: 9865) | NO: 9866) | NO: 9867) | NO: 9868) | NO: 9869) | NO: 9870) |
| Flg | U0954 | CTAGCGATT | CGGCCTCCT | AGCGGGCAG | GCATTGATC | ACGGGTTAG | CAAAAGAGG |
| E | 9 | CAAGGCGAT | TCAAACACA | TTTAGGACC | GCATTTTTA | CAGGGCAGA | CGGGTTCAT |
| | | GG | TT | AC | GCC | AT | GC |
| | | (SEQ ID | (SEQ ID | (SEQ ID | (SEQ ID | (SEQ ID | (SEQ ID |
| | | NO: 9871) | NO: 9872) | NO: 9873) | NO: 9874) | NO: 9875) | NO: 9876) |
| Fli | M3769 | TTTAGAAGT | CATACACGC | AGTGTGGTC | CCCCTAATA | CAAAAGATT | AATGGTTTT |
| M- | 1 | CGTTGATGA | TCACTTCAT | GCCTGTGGT | GTCTGTCAA | GAAGCAGAA | CCTATACCC |
| set | | GA | CG | GGAG | TCAT | GAGT | TTGA |
| A | | (SEQ ID | (SEQ ID | (SEQ ID | (SEQ ID | (SEQ ID | (SEQ ID |
| | | NO: 9877) | NO: 9878) | NO: 9879) | NO: 9880) | NO: 9881) | NO: 9882) |
| Fli | M3769 | GAGAGCAAA | CATACACGC | AGTGTGGTC | CCCCTAATA | CAAAAGATT | AATGGTTTT |
| M- | 1 | TCCTTATCC | TCACTTCAT | GCCTGTGGT | GTCTGTCAA | GAAGCAGAA | CCTATACCC |
| set | | AG | CC | GGAG | TCAT | GACT | TTGA |
| B | | (SEQ ID | (SEQ ID | (SEQ ID | (SEQ ID | (SEQ ID | (SEQ ID |
| | | NO: 9883) | NO: 9884) | NO: 9885) | NO: 9886) | NO: 9887) | NO: 9888) |
| Mur | U3279 | TTGAAACCC | TCAACTGAT | GCTAGGATT | TACGAGACA | ATAGGCATG | CCATTACAT |
| C | 4 | CAAAAGTTT | AGGTAATAT | TATGCCAAT | AAATAGGGA | CAGAATTTT | TTCGCCTC |
| | | TAC | CCC | TTA | TTT | TCC | (SEQ ID |
| | | (SEQ ID | (SEQ ID | (SEQ ID | (SEQ ID | (SEQ ID | NO: 9894) |
| | | NO: 9889) | NO: 9890) | NO: 9891) | NO: 9892) | NO: 9893) | |
| dna | M1004 | CGATAGATA | GGGCTTGTA | GTTTTAAAA | TTCTAAAAG | TAAGTCAAG | TTTTGGGGT |
| E | 0 | TTGTAGAAG | TTCATTTTG | ACGCCATAG | GTGGTAATC | CCATAAAAC | AAAAAGGCT |
| | | TCA | TAA | CCA | TTC | CAAA | GAA |
| | | (SEQ ID | (SEQ ID | (SEQ ID | (SEQ ID | (SEQ ID | (SEQ ID |
| | | NO: 9895) | NO: 9896) | NO: 9897) | NO: 9898) | NO: 9899) | NO: 9900) |
| Ser | X0501 | ATCTTTTTG | AGACAGCAC | CAGCCACAC | GTAAGGCGT | GCCCCATTA | AAAGGATAC |
| S | 7 | CCCTTGCTC | CAGTTTGAT | TTCAATGTC | TAGAAAAAT | AAATCCTTT | AAGGGGGA |
| | | ATA | AAA | TAT | ACC | TCT | (SEQ ID |
| | | (SEQ ID | (SEQ ID | (SEQ ID | (SEQ ID | (SEQ ID | NO: 9906) |
| | | NO: 9901) | NO: 9902) | NO: 9903) | NO: 9904) | NO: 9905) | |
| gly | P0096 | CTCGCTCCA | TTTTTTAGG | TGTTTGGAA | CTTTTGGGG | TTTGATAAA | TTTCAAAAC |
| | 0 | TTTTATCTT | GAGGATTGA | ATGCTGGTG | GAGTTTGAC | CGCCCACTT | GCTCACCTT |
| | | TTA | GAT | ATC | AAG | TTT | TTG |
| | | (SEQ ID | (SEQ ID | (SEQ ID | (SEQ ID | (SEQ ID | (SEQ ID |
| | | NO: 9907) | NO: 9908) | NO: 9909) | NO: 9910) | NO: 9911) | NO: 9912) |
| Glt | L1458 | TCTATTCTT | ATAATGAGT | ACAATAATA | AATTAGCCC | AACAACCGC | CTTCAGCGA |
| x | 0 | TTGATGCTC | TTGATCGTT | GGCTTTGTC | TTAAAATAG | TAAAATCAA | TACTAAAAG |
| | | TCT | ACG | TTC | ATG | AC | AT |
| | | (SEQ ID | (SEQ ID | (SEQ ID | (SEQ ID | (SEQ ID | (SEQ ID |
| | | NO: 9913) | NO: 9914) | NO: 9915) | NO: 9916) | NO: 9917) | NO: 9918) |
| Sig | M3769 | TAGGGGCGA | GCTGGATAA | TTTTTGGGG | GGCTGGTAA | AGGCTATTC | ATTCTCATC |
| 28 | 1 | TTGAAAACA | GGATTTGCT | GTATGCTAA | ATACTGGAT | AAGGTGGCT | AACGACTTC |
| (fliA) | | GC | CT | PA | AG | AAA | TAAA |
| | | (SEQ ID | (SEQ ID | (SEQ ID | (SEQ ID | (SEQ ID | (SEQ ID |
| | | NO: 9919) | NO: 9920) | NO: 9921) | NO: 9922) | NO: 9923) | NO: 9924) |
| Sig | M7344 | GCAGTTGGC | GAGAGCGAA | TGATTGTTG | AAAATCGGT | CTTTTCCTT | AAAACAAAC |
| 54 | 3 | GGTATTTGG | GTTTATGAG | GGTAGCTCT | CTGATGCTC | TCGCTTGAA | GCATCAAAA |
| | | TG | PA | CA | TTA | GA | AT |
| | | (SEQ ID | (SEQ ID | (SEQ ID | (SEQ ID | (SEQ ID | (SEQ ID |
| | | NO: 9925) | NO: 9926) | NO: 9927) | NO: 9928) | NO: 9929) | NO: 9930) |
| Mur | U1240 | CGCCCGAAT | TGATGCAAC | GGGATTTTA | CAAGCGGCA | TTTTTAAGG | TGGGTTTTA |
| I | 5 | GGATGAGTA | AAATGGACG | GAATACTTT | AGTGGAAGA | GCGTATTTT | AGGAATGTG |
| | | GG (SEQ | ACA | TGC (SEQ | TA (SEQ | TGT (SEQ | ATG (SEQ |
| | | ID | (SEQ ID | ID | ID | ID | ID |
| | | NO: 9931) | NO: 9932) | NO: 9933) | NO: 9934) | NO: 9935) | NO: 9936) |
| dna | D2618 | CGCGCTCAA | GGCCCATTC | GCCCCATTC | CGCTTTAAC | AGCGTTTTT | TCCCTATCA |
| B | 5 | AATCCCTAA | TTTCGGATA | CTGTTTTTA | GCTCCTTTC | GTAAGGGGG | TAGCGTTAG |
| | | AT | TT | GC | AC | TAT | TGC |
| | | (SEQ ID | (SEQ ID | (SEQ ID | (SEQ ID | (SEQ ID | (SEQ ID |
| | | NO: 9937) | NO: 9938) | NO: 9939) | NO: 9940) | NO: 9941) | NO: 9942) |
| Mur | D1060 | CTTAGGGGT | GCACAATTC | CCAAAGCTA | GCTCATGGA | CTTAGCCCC | CGCAAAAGG |
| G | 2 | TTTTAGCAT | CCACACGCT | AAGCGGTGT | TATAAAGGG | TTTAGTGTT | GTAGGGGAT |
| | | GAA | GC | TTT | GTATT | TA | PA |
| | | (SEQ ID | (SEQ ID | (SEQ ID | (SEQ ID | (SEQ ID | (SEQ ID |
| | | NO: 9943) | NO: 9944) | NO: 9945) | NO: 9946) | NO: 9947) | NO: 9948) |
| Ipx | U3279 | TTTTATTTT | CAAACTTAT | AAAGATAAC | TAATTCTAC | GCGGTCATG | ATTCAAAGA |
| C | 4 | TAGAAACGA | CGCCCTCTC | GCTAGGATT | AGAGTGGTT | GAATTTTTA | AAGCTGGCT |
| | | ATC | TA | TCTAC | AATGG | GA | GTCT |
| | | (SEQ ID | (SEQ ID | (SEQ ID | (SEQ ID | (SEQ ID | (SEQ ID |
| | | NO: 9949) | NO: 9950) | NO: 9951) | NO: 9952) | NO: 9953) | NO: 9954) |
| kdt | M8630 | GCTTGTGGG | GAACCCCCT | ACCATGCTC | GTAAGTTTG | AAAAAGAAA | AAAGATACT |
| A | 5 | GGTTGTTTT | AAAATGACA | ATTAACGCT | AGCGGCTAA | GAAGAACTC | CCCCTGTGA |
| | | AT | AT | AGG | TTC | GTG | TTA |
| | | (SEQ ID | (SEQ ID | (SEQ ID | (SEQ ID | (SEQ ID | (SEQ ID |
| | | NO: 9955) | NO: 9956) | NO: 9957) | NO: 9958) | NO: 9959) | NO: 9960) |
| Ipx | U0954 | CAAAGAAAC | GCATGGTAT | GCAGCGGCA | CGCCCCCAA | AAAGGTTTG | CACTTGAGC |
| B | 9 | GCAAAATAC | TCAGCGTTT | CAGCGACTT | AAAGTCGCA | AAACAAGAA | GTTAGCAAC |
| | | AG | TC | TAG | GTA | ATCT | AAT |
| | | (SEQ ID | (SEQ ID | (SEQ ID | (SEQ ID | (SEQ ID | (SEQ ID |
| | | NO: 9961) | NO: 9962) | NO: 9963) | NO: 9964) | NO: 9965) | NO: 9966) |
| KO | | GGGGTGTTT | CGCTAGGAG | CAAGGGCGT | GGGATTGTT | GGAATACAA | GCCTTTTTA |
| 24 | | GAAATTGAT | AAAGGAAGG | TTTTTGGGG | ACAGGAAAA | TAACGCATA | GACAACCCT |
| | | AGA | AAA | TAT | GAT | AAT | ACT |
| | | (SEQ ID | (SEQ ID | (SEQ ID | (SEQ ID | (SEQ ID | (SEQ ID |
| | | NO: 9967) | NO: 9968) | NO: 9969) | NO: 9970) | NO: 9971) | NO: 9972) |
| KO | | CCCCTAAAC | GCTAGAAAT | GCGATTATG | TTATTGTGG | TATGCGGCT | CCCTAAATC |
| 26 | | TCAAATCTC | GCCATGAGA | GGGTATTTA | AGTTGCTTG | CATCCTATT | CAAATCAAG |
| | | AAT | AAG | TTG | TCA | AAA | CAG |
| | | (SEQ ID | (SEQ ID | (SEQ ID | (SEQ ID | (SEQ ID | (SEQ ID |
| | | NO: 9973) | NO: 9974) | NO: 9975) | NO: 9976) | NO: 9977) | NO: 9978) |
| K02 | | CCCCCTAAC | CAAACGATA | TCCAGCTTG | GGTGGTGAT | TTTGGTTGC | TTTTGACAG |
| 7 | | CCCATAATA | GAAAGAAAT | AAATCCAAA | TAAAAACCT | TTGGTTTGA | ACGATGACA |
| | | CCG | ACG (SEQ | TCC (SEQ | TATTG | TAG (SEQ | GAA (SEQ |
| | | (SEQ ID | ID NO: | ID NO: | (SEQ ID | ID NO: | ID NO: |
| | | NO: 9979) | 9980) | 9981) | NO: 9982) | 9983) | 9984) |
| fts | | TTTGAGGGT | TGGTTTTAC | ATTTTGATT | CGCTTTTTT | AAGATTACG | CGCTTTTTT |
| Y | | GCTTATTTT | CCGCCCCAT | GGTTTTGAT | TCTTTAACC | CCCCCACCT | ATGCTGTTT |
| | | ACT | TA | ATTCA | TCTTC | TTA (SEQ | TAA (SEQ |
| | | (SEQ ID | (SEQ ID | (SEQ ID | (SEQ ID | ID NO: | ID NO: |
| | | NO: 9985) | NO: 9986) | NO: 9987) | NO: 9988) | 9989) | 9990) |
| 26K | | AATACGCTT | TCCACCATG | CACTTGTTC | GACTTGCCA | CCCACTTTA | CAAGCAGTA |
| Da_ | | GAATATGTT | TTTCCGCTG | GCTGTCAAT | TTAGGTAGG | AGCTCACGA | AAAATGAAA |
| pro | | CAT | ATT (SEQ | AG (SEQ | AA (SEQ | GTGA (SEQ | CTAT (SEQ |
| t | | (SEQ ID | ID NO: | ID NO: | ID NO: | ID NO: | ID NO: |
| | | NO: 9991) | 9992) | 9993) | 9994) | 9995) | 9996) |
| K02 | | GCTTTTTAG | ATAAGGTTT | CTCTTGTTG | CATTCGGGT | TAGGACTTC | TTTATCCAA |
| 8 | | CGTCATAGA | TGTTTGCGG | TATTCAATG | TATTTTGAC | ATAACTTTC | ATCATCTCT |
| | | TAT (SEQ | TAG (SEQ | AAACT | TAT (SEQ | ATCAT | CAAG (SEQ |
| | | ID NO: | ID NO: | (SEQ ID | ID NO: | (SEQ ID | ID NO: |
| | | 9997) | 9998) | NO: 9999) | 10000) | NO: | 10002) |
| | | | | | | 10001) | |
| VAC | | TTCTATTCG | ATCAATTTT | CGGCCCAAA | CGGTAAAAG | TTCTGGTGG | CCCATCCCA |
| 51 | | CCTAAAACC | CAGCCCTAA | ATTAGACAG | CGTTCGCAT | TTTAGCTCG | CCCCTAGAG |
| | | TAC (SEQ | CTA (SEQ | CC (SEQ | CC (SEQ | TTTGG | ATCC (SEQ |
| | | ID NO: | ID NO: | ID NO: | ID NO: | (SEQ ID | ID NO: |
| | | 10003) | 10004) | 10005) | 10006) | NO: | 10008) |
| | | | | | | 10007) | |
| mur | | CTCGTTACA | GAATGCCAT | CGGGTTTAA | CTAACGCCC | CGAACATGC | GGCTCTGTT |
| B | | GCAGCGTGA | ATTCTTGTA | TGGCGTTGT | CTAAAGTGC | TTTTAGATT | CTCATCAAT |
| | | AA (SEQ | GC (SEQ | TT (SEQ | CA (SEQ | AC (SEQ | AG (SEQ |
| | | ID NO: | ID NO: | ID NO: | ID NO: | ID NO: | ID NO: |
| | | 10009) | 10010) | 10011) | 10012) | 10013) | 10014) |

Genomic DNA prepared from the Helicobacter pylori HpJ99 strain (ATCC 55679; deposited by Genome Therapeutics Corporation, 100 Beaver Street, Waltham, Mass. 02154) is used as the source of template DNA for amplification of the ORFs by PCR (polymerase chain reaction) (Current Protocols in Molecular Biology, John Wiley and Sons, Inc., F. Ausubel et al., editors, 1994). For the preparation of genomic DNA from H. pylori, see Example I. PCR amplification is carried out by introducing 10 nanograms of genomic HpJ99 DNA into a reaction vial containing 10 mM Tris pH 8.3, 50 mM KCl, 2 mM MgCl₂, 2 microMolar synthetic oligonucleotide primers (forward=F1 and reverse=R1), 0.2 mM of each deoxynucleotide triphosphate (dATP, dGTP, dCTP, dTTP), and 1.25 units of heat stable DNA polymerase (Amplitaq, Roche Molecular Systems, Inc., Branchburg, N.J., USA) in a final volume of 40 microliters. The PCR is carried out with Perkin Elmer Cetus/GeneAmp PCR System 9600 thermal cyclers.

TABLE 14
PCR Conditions

MurC (SEQ ID NO: 7608)
Denaturation at 94° C. for 2 min.,
25 cycles of 94° C. for 15 sec, 48° C. for 15 sec, 72° C. for 1 min, 30 sec,
Final Extension of 72° C. for 20 minutes.
Sig54 (SEQ ID NO: 7023)
Denaturation at 94° C. for 2 min.,
25 cycles of 94° C. for 15 sec, 50° C. for 15 sec, 72° C. for 1 min, 30 sec,
Final Extension of 72° C. for 20 minutes.
lpxC (SEQ ID NO: 7767)
Denaturation at 94° C. for 2 min.,
32 cycles of 94° C. for 15 sec, 50° C. for 15 sec, 72° C. for 1 min, 30 sec,
Final Extension of 72° C. for 20 minutes.
KO24,(SEQ ID NO: 8390; KO27 SEQ ID NO: 8850)
Denaturation at 94° C. for 2 min.,
25 cycles of 94° C. for 15 sec, 50.5° C. for 20 sec, 72° C. for 2 min,
Final Extension of 72° C. for 20 minutes.
KO29, (SEQ ID NO: 5811; 26 kDa Protein; SEQ ID NO: 5185)
Denaturation at 94° C. for 2 min.,
28 cycles of 94° C. for 15 sec, 50.5° C. for 20 sec, 72° C. for 2 min,
Final Extension of 72° C. for 20 minutes.
DnaE (SEQ ID NO: 7200),Glycyl (SEQ ID NO: 7200)
Denaturation at 94° C. for 2 min.,
20 cycles of 94° C. for 15 sec, 51° C. for 15 sec, 72° C. for 1 min, 30 sec,
Final Extension of 72° C. for 20 minutes.
Gltx (SEQ ID NO: 7021)
Denaturation at 94° C. for 2 min.,
25 cycles of 94° C. for 15 sec, 51° C. for 15 sec, 72° C. for 1 min, 30 sec,
Final Extension of 72° C. for 20 minutes.
KO28 (SEQ ID NO: 9375)
Denaturation at 94° C. for 2 min.,
25 cycles of 94° C. for 15 sec, 51° C. for 15 sec, 72° C. for 2 min,
Final Extension of 72° C. for 20 minutes.
KO30 (SEQ ID NO: 5216)
Denaturation at 94° C. for 2 min.,
25 cycles of 94° C. for 15 sec, 51.5° C. for 15 sec, 72° C. for 1 min, 45 sec,
Final Extension of 72° C. for 20 minutes.
MurI (SEQ ID NO: 7635), MurG (SEQ ID NO: 7605)
Denaturation at 94° C. for 2 min.,
25 cycles of 94° C. for 15 sec, 52° C. for 15 sec, 72° C. for 1 min, 30 sec,
Final Extension of 72° C. for 20 minutes.
DnaB (SEQ ID NO: 7419), KdtA (SEQ ID NO: 85610),LpxB (SEQ ID NO: 6957)
Denaturation at 94° C. for 2 min.,
27 cycles of 94° C. for 15 sec, 52° C. for 15 sec. 72° C. for 1 min, 30 sec,
Final Extension of 72° C. for 20 minutes.
Tsf (SEQ ID NO: 7109),FlgE (SEQ ID NO: 4820), FliM (SEQ ID NO: 4795),Sig28
(SEQ ID NO: 7310),MurB (SEQ ID NO: 8208)
Denaturation at 94° C. for 2 min.,
30 cycles of 94° C. for 15 sec, 52° C. for 15 sec, 72° C. for 1 min, 30 sec,
Final Extension of 72° C. for 20 minutes.
PpiB (SEQ ID NO: 7674)
Denaturation at 94° C. for 2 min.,
30 cycles of 94° C. for 15 sec, 52° C. for 15 sec, 72° C. for 2 min, 30 sec,
Final Extension of 72° C. for 20 minutes.
MurD (SEQ ID NO: 7597),MurE (SEQ ID NO: 7639), AlgA (SEQ ID NO: 6351),MetL
(SEQ ID NO: 9467),FusA (SEQ ID NO: 7042),SerS (SEQ ID NO: 7182),RnhA (SEQ
ID NO: 6992)
Denaturation at 94° C. for 2 min.,
30 cycles of 94° C. for 15 sec, 55° C. for 15 sec, 72° C. for 1 min, 30 sec,
Final Extension of 72° C. for 20 minutes.

Upon completion of thermal cycling reactions, each sample of amplified DNA is visualized on a 2% TAE agarose gel stained with Ethidium Bromide (Current Protocols in Molecular Biology, John Wiley and Sons, Inc., F. Ausubel et al., editors, 1994) to determine that a single product of the expected size had resulted from the reaction. Amplified DNA is then washed and purified using the Qiaquick Spin PCR purification kit (Qiagen, Gaithersburg, Md., USA).

PCR products are cloned into the pT7Blue T-Vector (catalog#69820-1, Novagen, Inc., Madison, Wis., USA) using the TA cloning strategy (Current Protocols in Molecular Biology, John Wiley and Sons, Inc., F. Ausubel et al., editors, 1994). The ligation of the PCR product into the vector is accomplished by mixing a 6 fold molar excess of the PCR product, 10 ng of pT7Blue-T vector (Novagen), 1 microliter of T4 DNA Ligase Buffer (New England Biolabs, Beverly, Mass., USA), and 200 units of T4 DNA Ligase (New England Biolabs) into a final reaction volume of 10 microliters. Ligation is allowed to proceed for 16 hours at 16° C.

Ligation products are electroporated (Current Protocols in Molecular Biology, John Wiley and Sons, Inc., F. Ausubel et al., editors, 1994) into electroporation-competent XL-1 Blue or DH5-a E. coli cells (Clontech Lab., Inc. Palo Alto, Calif., USA). Briefly, 1 microliter of ligation reaction is mixed with 40 microliters of electrocompetent cells and subjected to a high voltage pulse (25 microFarads, 2.5 kV, 200 ohms) after which the samples are incubated in 0.45 ml SOC medium (0.5% yeast extract, 2% tryptone, 10 mM NaCl, 2.5 mM KCl, 10 mM MgCl₂, 10 mM MgSO₄ and 20 mM glucose) at 37° C. with shaking for 1 hour. Samples are then spread onto LB (10 g/l bacto tryptone, 5 g/l bacto yeast extract, 10 g/l sodium chloride) plates containing 100 microgram/ml of Ampicillin, 0.3% X-gal, and 100 microgram/ml IPTG. These plates are incubated overnight at 37° C. Ampicillin-resistant colonies with white color are selected, grown in 5 ml of liquid LB containing 100 microgram/ml of Ampicillin, and plasmid DNA is isolated using the Qiagen miniprep protocol (Qiagen, Gaithersburg, Md., USA).

To verify that the correct H. pylori DNA inserts had been cloned, these pT7Blue plasmid DNAs are used as templates for PCR amplification of the cloned inserts, using the same forward and reverse primers used for the initial amplification of the J99 H. pylori sequence. Recognition of the primers and a PCR product of the correct size as visualized on a 2% TAE, ethidium bromide stained agarose gel are confirmation that the correct inserts had been cloned. Two to six such verified clones are obtained for each knock-out target, and frozen at −70° C. for storage. To minimize errors due to PCR, plasmid DNA from these verified clones are pooled, and used in subsequent cloning steps.

The sequences of the genes/ORFs are again used to design a second pair of primers which flank the region of H. pylori DNA to be either interrupted or deleted (up to 250 basepairs) within the ORFs but are oriented away from each other. The pool of circular plasmid DNAs of the previously isolated clones are used as templates for this round of PCR. Since the orientation of amplification of this pair of deletion primers is away from each other, the portion of the ORF between the primers is not included in the resultant PCR product. The PCR product is a linear piece of DNA with H. pylori DNA at each end and the pT7Blue vector backbone between them which, in essence, resultes in the deletion of a portion of the ORFs. The PCR product is visualized on a 1% TAE, ethidium bromide stained agarose gel to confirm that only a single product of the correct size has been amplified.

A Kanamycin-resistance cassette (Labigne-Roussel et al., 1988 J. Bacteriology 170, 1704-1708) is ligated to this PCR product by the TA cloning method used previously (Current Protocols in Molecular Biology, John Wiley and Sons, Inc., F. Ausubel et al., editors, 1994). The Kanamycin cassette containing a Campylobacter kanamycin resistance gene is obtained by carrying out an EcoRI digestion of the recombinant plasmid pCTB8:kan (Cover et al., 1994, J. Biological Chemistry 269, pp. 10566-10573). The proper fragment (1.4 kb) is isolated on a 1% TAE gel, and isolated using the QIAquick gel extraction kit (Qiagen, Gaithersburg, Md., USA). The fragment is end repaired using the Klenow fill-in protocol, which involved mixing 4 ug of the DNA fragment, 1 microliter of dATP, dGTP, dCTP, dTTP at 0.5 mM, 2 microliter of Klenow Buffer (New England Biolabs) and 5 units of Klenow DNA Polymerase I Large (Klenow) Fragment (New England Biolabs) into a 20 microliter reaction, incubating at 30° C. for 15 min, and inactivating the enzyme by heating to 75° C. for 10 minutes. This blunt-ended Kanamycin cassette is then purified through a Qiaquick column (Qiagen, Gaithersburg, Md., USA) to eliminate nucleotides. The “T” overhang is then generated by mixing 5 micrograms of the blunt-ended kanamycin cassette, 10 mM Tris pH 8.3, 50 mM KCl, 2 mM MgCl₂, 5 units of DNA Polymerase (Amplitaq, Roche Molecular Systems, Inc., Branchburg, N.J., USA), 20 microliters of 5 mM dTTP, in a 100 microliter reaction and incubating the reaction for 2 hours at 37° C. The “Kan-T” cassette is purified using a QIAquick column (Qiagen, Gaithersburg, Md., USA). The PCR product of the deletion primers (F2 and R2) is ligated to the Kan-T cassette by mixing 10 to 25 ng of deletion primer PCR product, 50-75 ng Kan-T cassette DNA, 1 microliter 10× T4 DNA Ligase reaction mixture, 0.5 microliter T4 DNA Ligase (New England Biolabs, Beverly, Mass., USA) in a 10 microliter reaction and incubating for 16 hours at 16° C.

The ligation products are transformed into XL-1 Blue or DH5-a E. coli cells by electroporation as described previously. After recovery in SOC, cells are plated onto LB plates containing 100 microgram/ml Ampicillin and grown overnight at 37° C. These plates are then replica plated onto plates containing 25 microgram/ml Kanamycin and allowed to grow overnight. Resultant colonies have both the Ampicillin resistance gene present in the pT7Blue vector, and the newly introduced Kanamycin resistance gene. Colonies are picked into LB containing 25 microgram/ml Kanamycin and plasmid DNA is isolated from the cultured cells using the Qiagen miniprep protocol (Qiagen, Gaithersburg, Md., USA).

Several tests by PCR amplification are conducted on these plasmids to verify that the Kanamycin is inserted in the H. pylori gene/ORF, and to determine the orientation of the insertion of the Kanamycin-resistance gene relative to the H. pylori gene/ORF. To verify that the Kanamycin cassette is inserted into the H. pylori sequence, the plasmid DNAs are used as templates for PCR amplification with the set of primers originally used to clone the H. pylori gene/ORFs. The correct PCR product is the size of the deleted gene/ORF but increased in size by the addition of a 1.4 kilobase Kanamycin cassette. To avoid potential polar effects of the kanamycin resistance cassette on H. pylori gene expression, the orientation of the Kanamycin resistance gene with respect to the knock-out gene/ORF is determined and both orientations are eventually used in H. pylori transformations (see below). To determine the orientation of insertion of the kanamycin resistance gene, primers are designed from the ends of the kanamycin resistance gene (“Kan-1” 5′-ATCTTACCTATCACCTCAAAT-3′ (SEQ ID NO:10015)), and “Kan-2” 5′-AGACAGCAACATCTTTGTGAA-3′ (SEQ ID NO:10016)). By using each of the cloning primers in conjunction with each of the Kan primers (4 combinations of primers), the orientation of the Kanamycin cassette relative to the H. pylori sequence is determined. Positive clones are classified as either in the “A” orientation (the same direction of transcription is present for both the H. pylori gene and the Kanamycin resistance gene), or in the “B” orientation (the direction of transcription for the H. pylori gene is opposite to that of the Kanamycin resistance gene). Clones which share the same orientation (A or B) are pooled for subsequent experiments and independently transformed into H. pylori.

Transformation of Plasmid DNA into H. Pylori Cells

Two strains of H. pylori are used for transformation: ATCC 55679, the clinical isolate which provided the DNA from which the H. pylori sequence database is obtained, and AH244, an isolate which had been passaged in, and has the ability to colonize the mouse stomach. Cells for transformation are grown at 37° C., 10% CO₂, 100% humidity, either on Sheep-Blood agar plates or in Brucella Broth liquid. Cells are grown to exponential phase, and examined microscopically to determine that the cells are “healthy” (actively moving cells) and not contaminated. If grown on plates, cells are harvested by scraping cells from the plate with a sterile loop, suspended in 1 ml of Brucella Broth, spun down (1 minute, top speed in eppendorf microfuge) and resuspended in 200 microliters Brucella Broth. If grown in Brucella Broth liquid, cells are centrifuged (15 minutes at 3000 rpm in a Beckman TJ6 centrifuge and the cell pellet resuspended in 200 microliters of Brucella broth. An aliquot of cells is taken to determine the optical density at 600 nm, in order to calculate the concentration of cells. An aliquot (1 to 5 OD₆₀₀ units/25 microliter) of the resuspended cells is placed onto a prewarmed Sheep-Blood agar plate, and the plate is further incubated at 37° C., 6% CO₂, 100% humidity for 4 hours. After this incubation, 10 microliters of plasmid DNA (100 micrograms per microliter) is spotted onto these cells. A positive control (plasmid DNA with the ribonuclease H gene disrupted by kanamycin resistance gene) and a negative control (no plasmid DNA) are done in parallel. The plates are returned to 37° C., 6% CO₂ for an additional 4 hours of incubation. Cells are then spread onto that plate using a swab wetted in Brucella broth, and grown for 20 hours at 37° C., 6% CO₂. Cells are then transferred to a Sheep-Blood agar plate containing 25 micrograms/ml Kanamycin, and allowed to grow for 3 to 5 days at 37° C., 6% CO₂, 100% humidity. If colonies appear, they are picked and regrown as patches on a fresh Sheep-Blood agar plate containing 25 micrograms/ml Kanamycin.

Three sets of PCR tests are done to verify that the colonies of transform ants have arisen from homologous recombination at the proper chromosomal location. The template for PCR (DNA from the colony) is obtained by a rapid boiling DNA preparation method as follows. An aliquot of the colony (stab of the colony with a toothpick) is introduced into 100 microliters of 1% Triton X-100, 20 mM Tris, pH 8.5, and boiled for 6 minutes. An equal volume of phenol: chloroform (1:1) is added and vortexed. The mixture is microfuged for 5 minutes and the supernatant is used as DNA template for PCR with combinations of the following primers to verify homologous recombination at the proper chromosomal location.

TEST 1. PCR with cloning primers originally used to amplify the gene/ORF. A positive result of homologous recombination at the correct chromosomal location should show a single PCR product whose size is expected to be the size of the deleted gene/ORF but increased in size by the addition of a 1.4 kilobase Kanamycin cassette. A PCR product of just the size of the gene/ORF is proof that the gene had not been knocked out and that the transformant is not the result of homologous recombination at the correct chromosome location.

TEST 2. PCR with F3 (primer designed from sequences upstream of the gene/ORF and not present on the plasmid), and either primer Kan-1 or Kan-2 (primers designed from the ends of the kanamycin resistance gene), depending on whether the plasmid DNA used was of “A” or “B” orientation. Homologous recombination at the correct chromosomal location will result in a single PCR product of the expected size (i.e., from the location of F3 to the insertion site of kanamycin resistance gene). No PCR product or PCR product(s) of incorrect size(s) will prove that the plasmid had not integrated at the correct site and that the gene had not been knocked out.

TEST 3. PCR with R3 (primer designed from sequences downstream of the gene/ORF and not present on the plasmid) and either primer Kan-1 or Kan-2, depending on whether the plasmid DNA used was of “A” or “B” orientation. Homologous recombination at the correct chromosomal location will result in a single PCR product of the expected size (i.e., from the insertion site of kanamycin resistance gene to the downstream location of R3). Again, no PCR product or PCR product(s) of incorrect size(s) will prove that the plasmid had not integrated at the correct site and that the gene had not been knocked out.

Transformants showing positive results for all three tests above indicate that the gene is not essential for survival in vitro.

A negative result in any of the three above tests for each transformant indicates that the gene had not been disrupted, and that the gene is essential for survival in vitro.

In the event that no colonies result from two independent transformations while the positive control with the disrupted ribonuclease H plasmid DNA produces transformants, the plasmid DNA is further analyzed by PCR on DNA from transformant populations prior to plating for colony formation. This will verify that the plasmid can enter the cells and undergo homologous recombination at the correct site. Briefly, plasmid DNA is incubated according to the transformation protocol described above. DNA is extracted from the H. pylori cells immediately after incubation with the plasmid DNAs and the DNA is used as template for the above TEST 2 and TEST 3. Positive results in TEST 2 and TEST 3 would verify that the plasmid DNA could enter the cells and undergo homologous recombination at the correct chromosomal location. If TEST 2 and TEST 3 are positive, then failure to obtain viable transformants indicates that the gene is essential, and cells suffering a disruption in that gene are incapable of colony formation.

Genes used in these experiments have been found to be essential, non-essential, or are still in progress, as indicated in Table 15.

TABLE 15
Summary
of knock-
out

| genes/ORFs | Accession | | |
|---|---|---|---|
| Gene | Number | Pathway | Status |
| rnh | P23329 | Transcription | Not essential |
| ppiB | P29820 | Translation | Not essential |
| tsf | P34828 | Translation | Essential |
| MurD | P14900 | Cell envelope | Essential |
| MurE | P22188 | Cell envelope | Essential |
| AlgA | P07874 | Virulence/Colo- | Not essential |
| | | nization | |
| metL | P19358 | Amino acid | Not essential |
| | | biosynthesis - | |
| | | aspartate | |
| | | family | |
| fusA | X16278 | Translation | Essential |
| FlgE | U09549 | Virulence/Colo- | Not essential- |
| | | nization | motility |
| | | | impaired |
| FliM | M37691 | Virulence/Colo- | Not essential- |
| | | nization | motility |
| | | | impaired |
| MurC | U32794 | Cell envelope | Essential |
| dnaE | M10040 | DNA | Essential |
| | | replication | |
| serS | X05017 | Translation | Essential |
| gly | P00960 | Translation | Essential |
| gltX | L14580 | Translation | Essential |
| sig28 | M37691 | Regulatory | Not essential- |
| (fliA) | | functions | motility |
| | | | impaired |
| sig54 | | Regulatory | Not essential- |
| | | functions | motility |
| | | | impaired |
| MurI | U12405 | Cell envelope | Essential |
| dnaB | D26185 | DNA | Essential |
| | | replication | |
| MurG | D10602 | Cell envelope | Essential |
| lpxC/envA | U32794 | Cell envelope | Essential |
| kdtA | M86305 | Cell envelope | Essential |
| lpxB | U09549 | Cell envelope | Essential |
| KO 24 | | thiolase-like | Not Essential |
| KO 26 | | histone-like | Not Essential |
| KO 27 | | respiratory | Not Essential |
| | | chain NADH | |
| | | dehydrogenase- | |
| | | like | |
| ftsY | P44870 | Cell division | Essential |
| 26 KDa | P21762 | Antioxidant | Essential |
| protein | | | |
| KO28 | | Aldose-1- | Not Essential |
| | | epimerase-like | |
| VAC51 | | Gram-neg | Not Essential |
| | | porin-like | |
| murB | P18579 | Cell wall | Essential |
| | | biosynthesis | |

VII. High-throughput Drug Screen Assay

Cloning, Expression and Protein Purification

Cloning, transformation, expression and purification of the H. pylori target gene and its protein product, e.g., an H. pylori enzyme, to be used in a high-throughput drug screen assay, is carried out essentially as described in Examples II and III above. Development and application of a screening assay for a particular H. pylori gene product, peptidyl-propyl cis-trans isomerase, is described below as a specific example.

Enzymatic Assay

The assay is essentially as described by Fisher (Fischer, G., et. al. (1984) Biomed. Biochim. Acta 43:1101-1111). The assay measures the cis-trans isomerization of the Ala-Pro bond in the test peptide N-succinyl-Ala-Ala-Pro-Phe-p-nitroanilide (Sigma # S-7388, lot # 84H5805). The assay is coupled with α-chymotrypsin, where the ability of the protease to cleave the test peptide occurs only when the Ala-Pro bond is in trans. The conversion of the test peptide to the trans isomer in the assay is followed at 390 nm on a Beckman Model DU-650 spectophotometer. The data are collected every second with an average scanning of time of 0.5 second. Assays are carried out in 35 mM Hepes, pH 8.0, in a final volume of 400 ul, with 10 μM α-chymotrypsin (type 1-5 from bovine Pancreas, Sigma # C-7762, lot 23H7020) and 10 nM PPIase. To initiate the reaction, 10 μl of the substrate (2 mM N-Succinyl-Ala-Ala-Pro-Phe-p-nitroanilide in DMSO) is added to 390 μl of reaction mixture at room temperature.

Enzymatic Assay in Crude Bacterial Extract.

A 50 ml culture of Helicobacter pylori (strain J99) in Brucella broth is harvested at mid-log phase (OD 600 nm ˜1) and resuspended in lysis buffer with the following protease inhibitors: 1 mM PMSF, and 10 μg/ml of each of aprotinin, leupeptin, pepstatine, TLCK, TPCK, and soybean trypsin inhibitor. The suspension is subjected to 3 cycles of freeze-thaw (15 minutes at −70° C., then 30 minutes at room temperature), followed by sonication (three 20 second bursts). The lysate is centrifuged (12,000 g×30 minutes) and the supernatant is assayed for enzymatic activity as described above.

Results

PPI from H. pylori was expressed in E. coli using the pET-28b expression vector from Novagen (cat # 69868-1). The expressed recombinant protein was isolated from the soluble fraction of bacterial cells that had been disrupted by cavitation in a Microfluidics Cell disruption chamber. The expression levels of recombinant PPI produced 100 mg of protein. The recombinant protein could be purified to homogeneity by Ni²⁺ chelate chromatography and gel filtration. On sodium dodecyl sulfate polyacrylamide gels, the recombinant protein migrates as a single band at 21 kDa, in accordance with the predicted molecular weight of 20,975 deduced from the gene sequence.

The PPIase activity was assayed using the chromogenic tetrapeptide substrate succinyl-Ala-Ala-Pro-Phe-p-nitroanilide. An initial velocity of 4.9 μmole/min/mg protein was measured with the purified enzyme (FIG. 5). This corresponds to a k_{cat} of 1.6 sec⁻¹ which is similar to the one obtained for the E. coli PPIase (Liu, J. and Walsh, C. T. (1990) Proc. Natl. Acad. Sci. USA 87:4028-4032) and the one from porcine kidney (Fischer, G. (1989) Nature 337:476-478).

The recombinant protein has a high catalytic efficiency of 2.06×10⁹ M⁻¹ s⁻¹ when the assay is measured at 25° C. These values are one to two orders of magnitude higher than that observed for other characterized PPIases. However, in those studies, the ppiase assay was conducted at 10° C., which may account for the discrepency. The calalytic efficiency is very close to the 1×10⁸ to 1×10⁹ M^{-1 s}⁻¹ upper diffusinal limit for “kinetically perfect” enzymes (Albery, W. J. and Knowles, J. R. (1976) Biochemistry 15:5631-5640) and suggests that by at least one measure, the H. pylori PPIase is a highly effective catalyst in the cis-trans isomerisation of the Ala-Pro bond in the oligopeptide substrate.

The presence of PPIase was also determined in an H. pylori extract. As with the assay for the recombinant protein, PPIase activity was detected, and was dependent on the concentration of extract added (FIG. 6).

These results show that PPIase activity can be measured on either H. pylori extracts or on the recombinant protein in E. coli. The high catalytic efficiency also demonstrates that H. pylori enzymes, such as PPIase, can be expressed at high levels and in an active form in E. coli. Such high yields of purified proteins provide for the design of various high throughput drug screening assays.

VIII. Cloning, Purification and Characterization of the Gene Encoding the Glutamate Racemase of H. Pylori.

The Helicobacter pylori genome contains an open reading frame (ORF) of 255 amino acids (SEQ ID NO: 7635) that was found to have homology to the Staphylococcus haemolyticus glutamate racemase gene (dga) (NCBI Accession number U12405) and to the E. coli murI gene which encodes glutamate racemase activity in that organism. To evaluate whether this H. pylori ORF encodes a protein with glutamate racemase activity, the gene was isolated by polymerase chain reaction (PCR) amplification cloning, overexpressed in E coli, and the protein purified to apparent homogeneity. A simple assay for glutamate racemase activity resulting in the isomerization of D-glutamic acid to L-glutamic acid was developed to facilitate purification and for future use as a high-throughput drug screen.

The ORF in H. pylori has been found by gene disruption studies to be essential for viability of H. pylori cells in laboratory culture (see Example VI above). Therefore, inhibition of the enzymatic activity would be expected to be lethal for the organism, and such inhibitors may have utility in antimicrobial therapy of human infectious diseases.

Cloning of H. pylori murI Gene Encoding Glutamate Racemase

A 765 base pair DNA sequence encoding the murI gene of H. pylori was isolated by polymerase chain reaction (PCR) amplification cloning. A synthetic oligonucleotide primer (5′-AAATAGTCATATGAAAATAGGCGTTTTTG-3′ (SEQ ID NO:10017)) encoding an NdeI restriction site and the 5′ terminus of the murI gene and a primer (5′-AGAATTCTATTACAATTTGAGCCATTCT-3′ (SEQ ID NO:10018)) encoding an EcoRI restriction site and the 3′ end of the murI gene were used to amplify the murI gene of H. pylori using genomic DNA prepared from the J99 strain of H. pylori as the template DNA for the PCR amplification reactions (Current Protocols in Molecular Biology, John Wiley and Sons, Inc. F. Ausubel et al., editors, 1994). To amplify a DNA sequence containing the murI gene, genomic DNA (25 nanograms) was introduced into each of two reaction vials containing 1.0 micromole of each synthetic oligonucleotide primer, 2.0 mM MgCl₂; 0.2 mM of each deoxynucleotide triphosphate (dATP, dGTP, dCTP & dTTP), and 1.25 units of heat stable DNA polymerases (Amplitaq, Roche Molecular Systems, Inc., Branchburg, N.J., USA) in a final volume of 50 microliters. The following thermal cycling conditions were used to obtain amplified DNA products for the murI gene using a Perkin Elmer Cetus/GeneAmp PCR System 9600 thermal cycler:

Conditions for Amplification of H. Pylori MurI (SEQ ID NO: 7635);

Denaturation at 94° C. for 2 min,

2 cycles at 94° C. for 15 sec, 30° C. for 30 sec and 72° C. for 15 sec

23 cycles at 94° C. for 15 sec, 53° C. for 30 sec and 72° C. for 15 sec

Reactions were concluded at 72° C. for 20 minutes

Upon completion of thermal cycling reactions, the amplified DNA was washed and purified using the Qiaquick Spin PCR purification kit (Qiagen, Gaithersburg, Md., USA). The amplified DNA sample was subjected to digestion with the restriction endonucleases, NdeI and EcoRI (New England Biolabs, Beverly, Mass. USA) (Current Protocols in Molecular Biology, Ibid). The DNA samples from each of two reaction mixtures were pooled and subjected to electrophoresis on a 1.0% SeaPlaque (FMC BioProducts, Rockland, Me., USA) agarose gel. DNA was visualized by exposure to ethidium bromide and long wave uv irradiation. Amplified DNA encoding the H. pylori murI gene was isolated from agarose gel slices and purified using the Bio 101 GeneClean Kit protocol (Bio 101 Vista, Calif., USA).

Cloning of H. Pylori DNA Sequences into the pET-23 Prokaryotic Expression Vector.

The pET-23b vector can be propagated in any E. coli K-12 strain, e.g., HMS174, HB101, JM 109, DH5α, etc., for the purpose of cloning or plasmid preparation. Hosts for expression include E. coli strains containing a chromosomal copy of the gene for T7 RNA polymerase. These hosts are lysogens of bacteriophage DE3, a lambda derivative that carries the lacI gene, the lacUV5 promoter and the gene for T7 RNA polymerase. T7 RNA polymerase is induced by addition of isopropyl-B-D-thiogalactoside (IPTG), and the T7 RNA polymerase transcribes any target plasmid, such as pET-28b, carrying its gene of interest. Strains used in our laboratory include: BL21 (DE3) (Studier, F. W., Rosenberg, A. H., Dunn, J. J., and Dubendorff, J. W. (1990) Meth. Enzymol. 185, 60-89).

The pET-23b vector (Novagen, Inc., Madison, Wis., USA) was prepared for cloning by digestion with NdeI and EcoRI (Current Protocols in Molecular Biology, Ibid). Following digestion, the amplified, agarose gel-purified DNA fragment carrying the murI gene was cloned (Current Protocols in Molecular Biology, Ibid) into the previously digested pET-23b expression vector. Products of the ligation reaction were then used to transform the BL21 (DE3) strain of E. coli.

Transformation of Competent Bacteria with Recombinant Plasmids

Competent bacteria, E coli strain BL21 or E. coli strain BL21(DE3), were transformed with recombinant pET23-murI expression plasmid carrying the cloned H. pylori sequence according to standard methods (Current Protocols in Molecular, Ibid). Briefly, 1 microliter of ligation reaction was mixed with 50 microliters of electrocompetent cells and subjected to a high voltage pulse, after which, samples were incubated in 0.45 milliliters SOC medium (0.5% yeast extract, 2.0% tryptone, 10 mM NaCl, 2.5 mM KCl, 10 mM MgCl₂, 10 mM MgSO₄ and 20 mM glucose) at 37° C. with shaking for 1 hour. Samples were then spread on LB agar plates containing 100 microgram/ml ampicillin for growth overnight. Transformed colonies of BL21 were then picked and analyzed to evaluate cloned inserts as described below.

Identification of Recombinant pET Expression Plasmids Carrying H. Pylori Sequences

Individual BL21 clones transformed with recombinant pET-23-murI were analyzed by PCR amplification of the cloned inserts using the same forward and reverse primers, specific for each H. pylori sequence, that were used in the original PCR amplification cloning reactions. Successful amplification verified the integration of the H. pylori sequences in the expression vector (Current Protocols in Molecular Biology, Ibid).

Isolation and Preparation of Plasmid DNA from BL21 Transformants Colonies carrying pET-23-murI vectors were picked and incubated in 5 mls of LB broth plus 100 microgram/ml ampicillin overnight. The following day plasmid DNA was isolated and purified using the Qiagen plasmid purification protocol (Qiagen Inc., Chatsworth, Calif., USA).

Cloning and Expression of the E. Coli groE Operon

It has been demonstrated that coexpression of the E. coli murI gene with the genes in the E. coli groE operon reduces the formation of insoluble inclusion bodies containing recombinant glutamate racemase (Ashiuchi, M., Yoshimura, T., Kitamura, T., Kawata, Y., Nagai, J., Gorlatov, S., Esaki, N. and Soda, K., 1995, J. Biochem. 117, 495-498). The groE operon encodes two proteins, GroES (97 amino acids) and GroEL (548 amino acids), which are molecular chaperones. Molecular chaperones cooperate to assist the folding of new polypeptide chains (F. Ulrich Hartl, 1996, Nature London 381, pp. 571-580).

The 2210 bp DNA sequence encoding the groE operon of E. coli (NCBI Accession number X07850) was isolated by polymerase chain reaction (PCR) amplification cloning. A synthetic oligonucleotide primer (5′-GCGAATTCGATCAGAATTTTTTTTCT-3′ (SEQ ID NO:10019)) encoding an EcoRI restriction site and the 5′ terminus of the groE operon containing the endogenous promoter region of the groE operon and a primer (5′-ATAAGTACTTGTGAATCTTATACTAG-3′ (SEQ ID NO:10020)) encoding a ScaI restriction site and the 3′ end of the groEL gene contained in the groE operon were used to amplify the groE operon of E. coli using genomic DNA prepared from E. coli strain MG1655 as the template DNA for the PCR amplification reactions (Current Protocols in Molecular Biology, Ibid). To amplify a DNA sequence containing the E. coli groE operon, genomic DNA (12.5 nanograms) was introduced into each of two reaction vials containing 0.5 micromoles of each synthetic oligonucleotide primer, 1.5 mM MgCl₂, 0.2 mM each deoxynucleotide triphosph te (dATP, dGTP, dCTP & dTTP) and 2.6 units heat stable DNA polymerases (Expanded High Fidelity PCR System, Boehringer Mannheim, Indianapolis, Ind.) in a final volume of 50 microliters. The following thermal cycling conditions were used to obtain amplified DNA products for the groE operon using a Perkin Elmer Cetus/GeneAmp PCR System 9600 thermal cycler:

Conditions for amplification and cloning of the E. coli groE operon;

Denaturation at 94° C. for 2 min,

2 cycles at 94° C. for 15 sec, 30° C. for 30 sec and 72° C. for 2 min

23 cycles at 94 C for 15 sec, 55° C. for 30 sec and 72° C. for 2 min

Reactions were concluded at 72° C. for 8 minutes

Upon completion of thermal cycling reactions, the amplified DNA was washed and purified using the Qiaquick Spin PCR purification kit (Qiagen, Gaithersburg, Md., USA). The amplified DNA sample was subjected to digestion with the restriction endonucleases, EcoRI and ScaI New England Biolabs, Beverly, Mass. USA) (Current Protocols in Molecular Biology, Ibid). The DNAs from each of two reaction mixtures were pooled and subjected to electrophoresis in a 1.0% SeaPlaque (FMC BioProducts, Rockland, Me., USA) agarose gel. DNA was visualized by exposure to ethidium bromide and long wave uv irradiation. DNA contained in slices isolated from the agarsoe gel was purified using the Bio 101 GeneClean Kit protocol (Bio 101 Vista, Calif., USA).

A DNA fragment, EcoRI to ScaI, containing the E coli groE operon was cloned into the corresponding sites of the pACYC184 expression vector (New England Biolabs, Beverly, Mass., USA) to make pACYC184-groE. The BL21 (DE3) strain of E. coli was transformed with pACYC-groE. A tetracycline-resistant transformant overexpressing proteins of Mᵣ˜14,000 (GroES) and Mᵣ˜60,000 (GroEL) was isolated.

Transformation of E. Coli Strain BL21(DE3) Carrying the pACYC-groE plasmid of E. coli.

Competent bacteria derived from a clone of strain BL21 (DE3) carrying the pACYC-groE plasmid were transformed with 50 nanograms of pET23-murI plasmid DNA, isolated as described above (Current Protocols in Molecular Biology, Ibid). A clone of BL21(DE3) carrying both the pACYC-groE expression plasmid and the pET-23-murI plasmid was isolated and used for expression of recombinant glutamate racemase as described below.

Expression of Recombiant H. Pylori murI (SEQ ID NO: 2873; SEQ ID NO: 7635)

A bacterial clone of BL21 (DE3) carrying both the pACYC-groE expression plasmid and the pET-23-murI plasmid was cultured in LB broth supplemented with 1.0 mM D,L-glutamic acid and 100 microgram/ml ampicillin and 10 micrograms/ml tetracycline at 30° C. until an optical density at 600 nM of 0.5 to 1.0 O.D. units was reached, at which point, isopropyl-beta-D-thiogalactoside (IPTG) was added to the culture at a final concentration of 1.0 mM. Cells were cultured overnight to induce gene expression of the H. pylori recombinant DNA constructions.

After induction of gene expression with IPTG, bacteria were pelleted by centrifugation in a Sorvall RC-3B centrifuge at 3000×g for 20 minutes at 4° C. Pellets were resuspended in 50 milliliters of cold 10 mM Tris-HCl, pH 8.0, 0.1 M NaCl and 0.1 mM EDTA (STE buffer). Cells were then centrifuged at 2000×g for 20 min at 4° C. Pellets were weighed (average wet weight =6 grams/liter) and processed to purify recombinant protein as described below.

Purification of Soluble Glutamate Racemase

All steps were carried out at 4° C. Cells were suspended in 4 volumes of lysis buffer (50 mM Potassium phosphate, pH 7.0, 100 mM NaCl, 2 mM EDTA, 2 mM EGTA, 10% glycerol, 10 mM D,L-glutamic acid, 0.1% β-mercaptoethanol, 200 μg/ml lysozyme, 1 mM PMSF, and 10 ug/ml each of leupeptin, aprotinin, pepstatin, L-1-chloro-3-[4-tosylamido]-7-amino-2-heptanone (TLCK), L-1-chloro-3-[4-tosylamido]-4-phenyl-2-butanone (TPCK), and soybean trypsin inhibitor, and ruptured by three passages through a small volume microfluidizer (Model M-110S, Microfluidics International Corporation, Newton, Mass.). The resultant homogenate was diluted with 1 volume of buffer A (10 mM Tris-HCl pH 7.0, 0.1 mM EGTA, 10% glycerol, 1 mM DL-Glutamic acid, 1 mM PMSF, 0.1% beta-mercaptoethanol), made 0.1% Brij-35, and centrifuged (100,000×g, 1 h) to yield a clear supernatant (crude extract).

After filtation through a 0.80-um filter, the extract was loaded directly onto a 20 ml Q-Sepharose column pre-equilibrated in buffer A containing 100 mM NaCl and 0.02% Brij-35. The column was washed with 100 ml (5 bed volumes) of Buffer A containing 100 mM NaCl and 0.02% Brij-35, then developed with a 100-ml linear gradient of increasing NaCl (from 100 to 500 mM) in Buffer A. A band of Mᵣ=28,000 corresponding to glutamate racemase, the product of the recombinant H. pylori murI gene, eluted at a gradient concentration of approximately 200-280 mM NaCl. Individual column fractions were then characterized for glutamate racemase activity (see below for description of assay) and the protein profile of the fractions were analyzed on 12% acrylamide SDS-PAGE gels.

Fractions containing glutamate racemase were pooled, brought to 70% saturation with solid (NH₄)₂SO₄, stirred for 20 min, and then centrifuged at 27,000×g for 20 min. The resulting pellet was resuspended in lysis buffer to a final volume of 8 ml and loaded directly onto a 350-ml column (2.2×92 cm) of Sephacryl S-100HR gel filtration medium equilibrated in buffer B (10 mM Hepes pH 7.5, 150 mM NaCl, 0.1 mM EGTA, 10% glycerol, 1 mM D,L-glutamatic acid, 0.1 mM PMSF, 0.1% beta-mercaptoethanol) and run at 30 ml/h. Fractions found to contain a glutamate racemase activity were pooled, and 0.5 volume of buffer C (10 mM Tris pH 7.5, 0.1 mM EGTA, 10% glycerol, 1 mM D,L-glutamic acid, 0.1 mM PMSF, 0.1% B-mercaptoethanol) was added (to reduce the NaCl concentration to 100 mM), and loaded onto a MonoQ 10/10 high-pressure liquid chromatography column equilibrated in buffer C containing 100 mM NaCl. The column was washed with 5 bed volumes of this buffer and developed with a 40 ml linear gradient of increasing NaCl (from 100 to 500). Glutamate racemase eluted as a sharp peak at 310 mM NaCl. Fractions containing a glutamate racemase activity were pooled, concentrated by dialysis against storage buffer [50% glycerol, 10 mM 3-(N-morpholino-propanesulfonic acid (MOPS) pH 7.0, 150 mM NaCl, 0.1 mM EGTA, 0.02% Brij-35, 1 mM dithiothreitol (DTT)], and stored at −20° C.

Assays for Glutamate Racemase Activity.

Conversion of D-glutamate to L-glutamate (Two Enzyme Coupled Assay)

The activity of glutamate racemase, interconversion of the enantiomers of glutamic acid, was measured using D-glutamic acid as substrate. The method of Gallo and Knowles (Gallo, K. A. and Knowles, J. R., 1993, Biochmistry 32, 3981-3990) that was used to measure the glutamate racemase activity of Lactobacillus fermenti was adapted for the measurement of glutamate racemase activity of the H. pylori murI gene product isolated as a recombinant protein from E. coli. In this assay, the measurement of the activity of glutamate racemase is linked to an OD change in the visible range in a series of coupled reactions to the activities of L-glutamate dehydrogenase (reduction of NAD to NADH) and diaphorase (reduction of the dye p-iodonitrotetrazolium violet, INT). Initial rates were determined by following the increase in absorbance at 500 nm in a reaction volume of 200 μl containing 50 mM Tris-HCl, pH 7.8, 4% v/v glycerol, 10 mM NAD, 2 mM INT, 60 Units/ml L-glutamate dehydrogenase, 5 Units/ml diaphorase, and varying concentrations of either substrate (from 0.063 mM to 250 mM D-glutamic acid) or purified enzyme (from 1 μg to 50 μg). After a preincubation of all reagents except either the substrate (D-glutamic acid) or the enzyme (murI gene product) for a period of 5 minutes, reactions were initiated by adding the missing ingredient (i.e., the enzyme or the subtrate, as required), and the increase in optical density at 500 nm was measured in a Microplate Spectophotometer System (Molecular Devices, Spectra MAX 250). Measurements were followed for 20 minutes, and initial velocities were derived by calculating the maximum slope for the absorbance increases. The coupled reaction can be summarized as shown below:

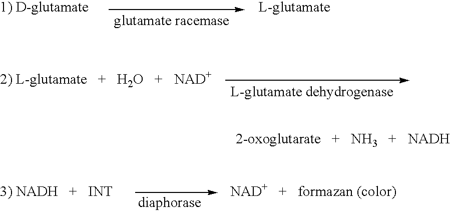

Conversion of D-glutamate to L-glutamate (Single Enzyme Coupled Assay)

In this assay, the conversion of D-glutamic acid to L-glutamic acid is coupled to the conversion of L-glutamic acid and NAD⁺by L-glutamate dehydrogenase to 2-oxoglutarate, ammonia. The production of NADH is measured as an increase of absorbance at 340 nm (the reduction of NAD⁺ to NADH) at 37° C. The standard assay mixture (adapted from Choi, S- Y,, Esaki, N., Yoshimura, T., and Soda, K., 1991, Protein Expression and Purification 2, 90-93) contained 10 mM Tris-HCl, pH 7.5, 5 mM NAD+, 5 Units/ml L-glutamate dehydrogenase, varying concentrations of the substrate D-Glutamic Acid (0.063 mM to 250 mM), and the purified recombinant H. pylori enzyme glutamate racemase (1 μg to 50 μg). The reaction was started by the addition of either the substrate D-glutamic acid or the recombinant glutamate racemase after a preincubation at 37° C. for 5 minutes with all of the other assay ingredients. The change in absorbance at 340 nm was measured in a Spectra MAX 250. Initial velocities were derived from the initial slopes. The coupled reactions can be summarized as shown below:

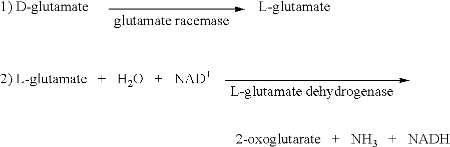

Results

1) Expression of the H. Pylori murI gene in E. Coli Cells

To examine its biochemical properties, the H. pylori glutamate racemase was overexpressed in E. coli and purified. In the presence of the E. coli chaperones GroES and GroEL, the glutamate racemase was expressed as a soluble protein. About 20 mg of soluble MurI (SEQ ID NO: 2873; SEQ ID NO: 7635) was produced per liter of culture as judged by intensity of the protein band after SDS-PAGE. No band corresponding to the molecular weight of murI protein was seen in control gel lanes containing extracts from cells transformed with the pET vector lacking a murI insert. Addition of 1 mM DL-glutamic acid during cultivation of the expressing cells increased the apparent expression level by about five-fold.

2) Purification of Recombinant H. Pylori murI Protein

MurI was purified by cation exchange chromatography and gel filtration. Upon SDS-PAGE analysis, the purified protein migrated as a single polypeptide species with an apparent mass 29 kDa which is consistent with the predicted mass of 28,858.

3) Kinetic Properties of Recombinant H. Pylroi murI enzyme

Kinetic constant for recombinant glutamate racemase were estimated by assaying its activity at various concentrations of protein and D-glutamic acid as described above. Purified recombinant H. pylori glutamate racemase exhibits a Vₘₐₓ of ˜300 nmoles/min/mg protein (kcat=8.6 min-1) and a Kₘ of ˜100 μM for D-glutamate. Although the Vmax value is lower than that observed for highly purified glutamate racemase from some other bacterial species, its Km for D-glutamic acid is higher than that observed for the enzyme from most other species, resulting in a catalytic efficiency (k_{cat}/Kₘ) which is typical of purified preparation from E. coli and P. Pentococcus.

4) Characterization of MurI: Inhibition by L-serine-O Sulfate

The H. pylori glutamate racemase was tested for inactivation with a sucuide inhibitor, L-serine-O sulfate, which is known to inhibit murI from E. coli. The enzyme was incubated in the presence of 20 mM L-serine-O sulfate, and at different times interval, aliquots were removed to determine residual activity. The initial velocity of purified recombinant H. pylroi murI protein was determined in the single enzyme coupled asssay following incubation with the inhibitor L-serine-O-sulfate (LSOS) at 20 mM for the times indicated on the x-axis. The control was incubated in an identical manner but without LSOS. As shown in FIG. 7, the H. pylori glutamate racemase can be readily inactivated by the inhibitor.

Future Application of the Glutamate Racemase Activity in High Throughput Drug Screening Assays.

The assays for measurement of H. pylori glutamate racemase activity described above have been carried out in 96-well plates in which multiple reactions were conducted simultaneously. Measurements of activity in a multi-well format are readily amenable to scale-up to permit rapid analysis of numerous compounds for inhibition of the glutamate racemase activity. Compunds which inhibit the activity of glutamate racemase may have application as novel antibiotics and may be suitable for the treatment and eradication of bacterial (e.g., H. pylori) infections in humans. Known inhibitors of glutamate racemase, such as L-serine-O-sulfate, can be used to calibrate high throughput screens of new compound libraries to facilitate identification of new compounds with properties suitable for in vivo human therapeutics.

IX. Cloning Purification, and Characterization of the Gene Encoding H. Pylori MurC (SEQ ID NO: 2845; SEQ ID NO: 7607).

Background on UDP-N-acetylmuramyl-alanine Synthetase (MurC)

MurC catalyzes peptide bond formation between the lactyl ether of UDP-N-acetylmuramate and L-alanine to form UDP-N-acetylmuramyl-alanine in one of the initial steps of bacterial peptidoglycan biosynthesis. The enzyme couples the formation of an amide bond with the cleavage of ATP to ADP and inorganic phosphate.

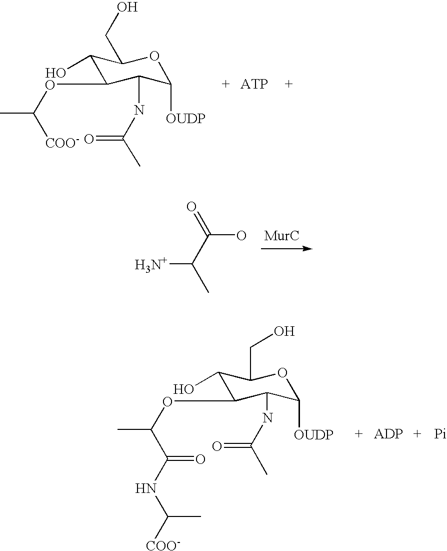

Peptidoglycan biosynthesis is both essential and unique to bacteria. A variety of bactericidal agents exist which interrupt peptidoglycan biosynthesis (e.g., lactams, vancomycin, phosphonomycin, D-cycloserine, alafosfalin, halovinylglycines, liposydomycin B). These compounds act at various steps in the pathway and all cause the induction of cell lysis. MurC is one of two cell wall enzymes (the other is the alanine racemase) which are thought to be inhibited by the cell wall active agent alafosfalin.

The assembly of the peptide moiety of the peptidoglycan monomer unit in E. coli is carried out by the stepwise addition of L-alanine, D-glutamate, diaminopimelate and D-alanine-D-alanine onto UDP-N-acetylmuramate catalyzed by four adding enzymes (MurC, MurD, MurE and MurF, respectively). The H. pylori genome contains a 475 amino acid open reading frame with relatively high homology to MurC proteins from H. influenzae and E. coli with lower similarity to that from P. gingivalis. Alignment of H. pylori MurC with these homologs reveals an overall identity of 14%.

Overproduction and Purification of Functional H. Pylori MurC (SEQ ID NO: 7607)

The H. pylori enzyme has been successfully overproduced in E coli and purified to yield functional enzyme of the appropriate molecular weight as evidenced by SDS-PAGE. Assay of the MurC protein relies on enzymatic synthesis of the substrate, UDP-N-acetylmuramate which is accessible by linking the two preceding enzymes in the peptidoglycan biosynthetic pathway. That procedure involves the incubation of purified E. coli MurA and MurB with commercially available UDP-N-acetylglucosamine and phosphoenolpyruvate followed by reverse phase HPLC purification of the product UDP-N-acetylmuramic acid. Assay of the UDP-N-acetylmuramate synthetase activity of the MurC protein is possible through any of the three methodologies: (i) substrates and products can be monitored by reverse phase HPLC; (ii) ADP production can be monitored continuously through coupled assay with pyruvate kinase and lactate dehydrogenase by following the stoichiometric oxidation of NADH; and (iii) inorganic phosphate produced in the reaction can be monitored spectrophotometrically by complex with molybdate in the presence of the dye malachite green. The inorganic phosphate assay will be most amenable to high throughput screening against compound libraries.

Cloning and Over-expression of H. Pylori MurC (SEQ ID NO: 7607)

Two primers were designed for the PCR cloning of murC, MURCE5 GCCATATGCTTGAAACCCCAAAAGTTTTACTCAAAAACC (SEQ ID NO:10021)) and MURCE3 GCGAATTCGCTCGCTCCTATAATCCCTACG (SEQ ID NO:10022)). The 5′ primer was designed incorporating an NdeI site, and the 3′ primer was designed incorporating an EcoRI site. These restriction sites were chosen to allow for the cloning of the entire murC gene into the expression vectors pET-23a and pET-28b, the latter vector allowing for the incorporation of a His tag into the over-expressed recombinant protein to aid in the purification process. Initial stability problems were experienced when attempts were made to clone the murC gene into a high copy number vector, therefore, the murC PCR product was blunt end cloned into the EcoRV site of the low copy number pOK12 plasmid prior to insertion into the expression vectors. The murC gene was excised from pOK12 using NdeI and EcoRI, and ligated into the compatible sites within pET-23a and pET-28b. The complete murC gene was then sequenced to ensure that no aberrant mutation had occurred during the PCR process, and that the primary amino acid sequence of the isolated recombinant protein was identical to the native protein.

Purification and Assay for H. Pylori MurC (SEQ ID NO: 7607)

Approximately 15 mg of His-tagged MurC was purified to homogeneity from a six liter fermentation of E coli strain BL21 DE3/pET28b-HpMurC according to the following procedure. Freshly transformed cells (6L) were grown to an optical density (600 nm) of 0.5 in Luria-Bertani media. These cells were then induced with 1 mM isopropylthiogalactoside, allowed to grow for another 3 hours and harvested by centrifugation. Cells were resuspended in 75 ml of 100 mM Tris·HCl, pH 8, 2.5 mM 2-mercaptoethanol and 10% (vol/vol) glycerol. To that mixture, lysozyme was added to a final concentration of 0.2 mg/ml and allowed to incubate for 10 min at 4° C. with gentle mixing before two passages at 2000 psi through a French Press. Cell debris and membranes were then pelleted by centrifugation at 200,000 g for 60 min. The resulting supernatant was applied to a Pharmacia HiTrap chelating nickel-charged column (10 ml) and washed with 5 column volumes of 20 mM sodium phosphate, 0.5 M NaCl, 10 mM imidazole, pH 7.5. The nickel column was then eluted with 500 mM imidizole in the above buffer. The nickel column eluate was subsequently dialyzed overnight against 50 mM Tris·HCl, 2.5 mM 2-mercaptoethanol, 10% glycerol, pH 8.0. The dialysate was then loaded onto a 15 ml Resource Q (Pharmacia) anion exchange column equilibrated in 50 mM Tris·HCl, 2.5 mM 2-mercaptoethanol, 10% glycerol, pH 8.0 and washed with ten column volumes of the same buffer. The column was then subjected to a 120 ml gradient from 0 to 500 mM KCl. MurC eluted with about 150 mM KCl and a total volume of 22 ml was quick-frozen with liquid nitrogen in 0.5 ml aliquots for subsequent assay.

UDP-N-acetylmurmate was synthesized enzymatically with partially purified E. coli MurA and MurB from overproducing strains described previously (Benson et al., 1993, Biochemistry 32:2024; Brown et al., 1994, Biochemistry 33:10638). The enzymatic synthesis involved purification of a 1 hour incubation of 0.5 mg MurA, 0.5 mg MurB, 20 mM UDP-GlcNAc, 20 mM PEP, 10 mM KCl; 20 mM NADPH, 5 mM DTT, 100 mM Tris·HCl, pH 8. UDP-N-acetylmurmate was purified from the mixture by isocratic reverse phase HPLC in 50 mM ammonium formate buffer pH 3.7 on a 10×250 mm, 5 μm C-18 column from YMC (Japan).

Purified H. pylori MurC was assayed using a continuous assay by coupling ADP formation with NADH oxidation by the pyruvate kinase/lactate dehydrogenase enzyme couple. Assay conditions were as follows: 100 mM Tris·HCl pH 8.0, 2.5 mM 2-mercaptoethanol, 20 mM KCl, 10 mM DTT, 2 mM MgCl₂, 10 mM PEP, 0.15 mM NADH, 3.5 units of pyruvate kinase, 5.5 units of lactate dehydrogenase (Sigma), 500 μM UDP-N-acetylmuramate, 1 mM L-alanine, 1 mM ATP and 0.0018 mg/ml MurC at 37° C. Under these conditions H. pylori MurC exhibited a specific activity of 5900 nmol/min/mg. In this assay ADP production was shown to be dependent on UDP-N-acetylmuramate, L-alanine and enzyme. Previously boiled enzyme could not support ADP formation. Steady state analysis of the dependence of reaction velocity on substrate concentration was done by varying the concentration of a single substrate in concentrations ranging from 0.5 to 5 times Kₘ while holding the other two substrates constant at 5 times Kₘ concentrations. Apparent Kₘ's for L-alanine, ATP and UDP-N-acetylmuramate were 80, 100 and 201M, respectively.

Based on the above, high throughput drug screening assays which measure the UDP-N-acetylmuramyl-alanine synthetase activity can be performed. The assays for the measurement of the H. pylori MurC activity can be carried out in a 96-well plate amenable for HTS. Such an assay will result in hits that can then be developed into drugs by those skilled in the art.

X. Expression Partial Purification and Enzymatic Measurement of the H. Pylori 3-deoxy-D-manno-2-octulosonate-8-phosphate Synthase.

Background on 3-deoxy-D-manno-2-octulosonic Acid (KDO)

3-deoxy-D-manno-2-octulosonic acid (KDO) is an unusual 8-carbon sugar found in the lipopolysaccharides of a wide variety of Gram-negative bacteria. It plays a crucial role in the assembly process of lipopolysaccharides providing a link between lipid A and the growing polysaccharide chain (Inouye, 1979). It has been shown that an interruption of the production and utilization of KDO leads to a buildup of lypopolysaccharide precursors and growth inhibition (Ghalambor et al., 1966; Mauson et al., 1978). Since KDO is-a molecule found only in Gram-negative organisms and is required for lipopolysacchirides synthesis and cell growth, the inhibition of one of the enzyme(s) responsible for the biosynthesis of KDO represent an attractive target for the discovery of a novel class of antibiotics directed against Helicobacter pylori infections.

There are at least four enzymes involved in the synthesis and utilization of KDO. The actual precursor is its 8-phosphate (KDO-8-P), which is formed by the unusual condensation of D-arabinose 5-phosphate (Ara5P) and phosphoenolpyruvate (PEP), catalyzed by the KDO-8-P synthase (EC 4.1.2.16). The available evidence supports the following overall reaction for KDO-8-P formation:

Ara5P+PEP KDO-8-P+Pi

KDO-8-P is then dephosphorylated by the next enzyme in the pathway to give KDO which is subsequently activated to cytidine-5-monophosphate-KDO and transferred to a lipopolysaccharide percursor.

Expression and Analysis of Soluble KDO-8-P Synthase (SEQ ID NO: 6945)

Recombinant H. pylori KDO-8-P synthase was expressed in Escherichia coli using the pET28b vector, that carries the strong T7 expression system, to express a histidine-tagged fusion protein. We have investigated the expression of soluble material at 25, 30 and 37° C. Cells were grown to an OD₆₀₀ of 0.6 in LB broth supplemented with 30 ug/ml kanamycin and 10 mM Potassium Phosphate pH 7.0, at which point, β-D-thiogalactopyranoside (IPTG) was added to a final concentration of mM. Cells were induced for 16 hours for 25′-C, 4 hours for 30° C., and 3 hours for 37° C. Cultures were harvested by centrifugation (20 min. at 3000×g, 4-C), washed with STE buffer (10 mM tris(hydroxymethyl)aminimethane (Tris)-HCl pH 8.0, 100 mM NaCl, 1 mM EDTA), and the cell pellets were stored at −70° C. A 1 litre culture of bacteria typically yielded 2 to 3 g (wet weight) of cells.

Analysis of Cell Fractionation

All steps were carried out at 4° C. Frozen cells were thawed, resuspended in five volumes of lysis buffer (50 mM Phosphate, pH 8, 0.5 M NaCl, 5 mM imidazole) with 10% glycerol, 10 mM PEP, 0.1% β-mercaptoethanol, 1 mM phenylmethylsulfonyl fluoride (PMSF), and 10 ug/ml each of leupeptin, aprotinin, pepstatin, L-1-chloro-3-[4-tosylamido]-7-amino-2-heptanone (TLCK), L-1-chloro-3-[4-tosylamido]-4-phenyl-2 butane (TPCK), and soybean trypsin inhibitor (Boehringer-Mannhein), and ruptured by several passages through a small volume microfluidizer (Model M-11OS, Microfluidics International Corporation, Newton, Mass.). Brij 35 was added to the resultant homogenate to a concentration of 0.1%, and the extract was centrifuged (100,000×g for 1 hour) to yield a clear supernatant (crude extract). The protein concentrations of both the soluble fraction and the cell pellet fraction were measured according to the method of Bradford (1976) and 15-g of soluble fractions and 4-g of cell pellets were analyzed by 12% sodium dodecyl sulfate polyacrylamide gel electrophoresis (SDS-PAGE) electrophoresis.

Expression of soluble recombinant KDO-8-P synthase was similar in all temperatures studied. Note that the insoluble fractions were overloaded relative to the soluble fraction (about 7-fold), therefore the amount of insoluble KDO-8-P synthase may be an overestimate relative to the soluble fractions.

Purification

Cell fractions containing recombinant H. pylori KDO-8-P synthase were filtered through a 0.45-m filter, the soluble fraction was loaded directly onto a 5-ml Ni²⁺ nitrolotriacetate-agarose (HiTrap Chelating, Pharmacia) (Hochuli et al. 1987) pre-equilibrated in lysis buffer containing 10% glycerol, 0.1% Brij 35 and 1 mM PMSF. The column was washed with 100 ml (20 bed volumes) of lysis buffer containing 10% glycerol, 0.1% Brij 35, and developed with a 60 ml linear gradient from 5 mM to 500 mM imidazole in lysis buffer containing 10% glycerol, 0.1% Brij 35 and 1 mM PMSF. Fractions were monitored by absorbance at OD280 nm, and peak fractions were analyzed by SDS-PAGE gel electrophoresis. Fractions containing the recombinant protein eluted at a concentration of 125 mM imidazole.

Assay 1

The enzyme reaction was performed in a final volume of 4001 containing 0.3 mM Ara5P, 0.3 mM PEP and 0.1 M Tris-acetate pH 7.5. Reactions were started with 10 g of purified enzyme and were followed spectrophotometrically at 232 nm for 15 minutes. As a control, the mixture minus enzyme was measured. No decrease in the absorbance at 232 nm was observed when no enzyme was added. Addition of 10 g KDO-8-P synthase resulted in a decrease of the absorbance at 232 nm, representing the utilization of PEP by the enzyme. An extinction coefficient of 2840 M cm⁻¹ of PEP, was used to estimate that at room temperature, the activity of the enzyme was 0.7-mole/min/mg protein.

Assay 2

Because of the potential interference of test compounds on the assay at 232 nm, a method was developed to measure the amount of Pi released in the reaction (Lanzatta et al. 1979).

This assay ws performed in a 96 well ELISA place, 50 ul assay mixtures containing 0,1 M Tris-acetate (pH 7.5), 0.3 mM Ara5P and 0.3 mM PEP, and 1.28 ug/ml of KDO-8-P synthase were incubated at 25° C. for different times. The reaction was then quenched with the addition of the molybdate-malachite green reagent. As shown in FIG. 8, the reaction proceeded linearly for 60 minutes. Under those conditions, the activity measured was 0.734-moles/min/mg protein, which was similar to the rate that was measured with the 232 nm assay.

Stimulation of the Reaction with Brij 35

When assayed in the presence of 0.02% Brij 35, the reaction proceeded faster. Under those conditions, the activity increased up to 1 mole/min mg protein.

Stability of the Enzyme

The long term storage stability of the enzyme was tested over several conditions. When the enzyme was present in a buffer containing 50 mM phosphate pH 8.0 and 1 mM PEP, the enzyme could be kept at −20° C. with no loss of activity for at least three weeks. Enzyme stored in 50% glycerol appeared to have less activity during extended storage at −20° C. (FIG. 9).

XI. Over-production and Purification of Enzymatically Active H. Pylori Asd.

The asd enzyme of H. pylori has been over-produced successfully in E. coli and purified in sufficient quantities to allow assay development for high through-put screening. The purified protein was determined to be Asd by monitoring its migration of SDS-PAGE gels to determine its molecular mass and by determining the enzymatic activity of the protein.

Cloning of the H. Pylori and E. Coli asd Genes.

The open reading frame encoding Asd (SEQ ID NO: 1783; SEQ ID NO: 6545) was identified from the genomic sequence of H. pylori by its coding sequence homology to the Asd enzyme of a number of other bacteria. Two oligonucleotide primers were designed to allow the amplification by PCR of the H. pylori asd gene from total H. pylori chromosomal DNA. The Hpasd1 primer 5′-AAACATATGAAGACTTATAATGTCGCTATTG-3′ (SEQ ID NO: 10023) was designed to contain an Nde 1 restriction site to facilitate cloning of the product into expression vectors. For the same reason, the Hpasd3 primer 5′-TTTGGATCCTTTAAGCAAGCTCAAGCGTTC-3′ (SEQ ID NO: 10024) contained a restriction site for Bam HI. The expression vectors chosen for expression were pET28b and pET30a. pET28b allows the addition of a His-tag to the N-terminus of Asd to facilitate purification.

The asd gene was amplified by PCR using the following conditions;

Initial denaturation at 94° C. for 5 min followed by 30 cycles of

Denaturation at 94° C. for 30 sec.

Annealing at 50° C. for 30 sec

Extension at 72° C. for 1 min

A final extension period of 7 min at 72° C. was also performed.

The product of the PCR was purified using the QIAEX II gel purification system (Qiagen, Chatsworth Calif.) and ligated overnight at 16° C. to the cloning vector pGEM-T (Promega, Madison Wis.) and transformed into E. coli DH5α. Resulting colonies were screened for the presence of the appropriate insert by restriction digest analysis. One clone containing the inserted asd gene was selected, digested with Nde I and Bam HI and run on an agarose gel. The band corresponding to the asd gene was cut from the gel, purified with the QIAEX II kit (Qiagen ibid.) and ligated overnight at 16° C. to either pET28b or pET30a. Clones containing the asd gene were identified by a white colony appearance on agar plates containing X-Gal. Appropriate constructs were confirmed by restriction digest analysis and by DNA sequencing of the entire insert.

The asd gene of E. coli was amplified using primers Ecasd35′-AAAGGTACCATGAAAAATG TTGGTTTTATCGGC-3′ (SEQ ID NO: 10025) and Ecasd25′-CCAGAATTCATGAATAAAGATTACGCCAG-3′ (SEQ ID NO: 10026) with total E. coli chromosomal DNA as a template. The PCR conditions were as described above for the H. pylori asd gene. The Ecasd1 primer was designed to contain a Kpn I restriction site to facilitate cloning of the product into expression vectors. For the same reason, the Ecasd2 primer contained the restriction site for EcoRI. The amplified band containing E. coli asd was isolated from the gel, purified with QIAEX (ibid.), ligated into pGEM-T and transformed into E. coli DH5α. Colonies were screened for the presence of the appropriate insert by restriction digest analysis. One clone containing the inserted asd gene was selected, digested with Kpn I and Eco RI and run on an agarose gel. The band corresponding to the E. coli asd gene was cut from the gel, purified with the QIAEX II kit (Qiagen, ibid.) and ligated overnight at 16° C. to pET28b.

Expression of E. Coli and H. Pylori Asd

The pET28b plasmid containing either E. coli or H. pylori asd (SEQ ID NO: 1783; SEQ ID NO: 6545) was transformed into E. coli HMS174 pLysS for expression of the protein. Cells were grown in LB medium supplemented with 50 mg/ml kanamycin (Fisher, Pittsburgh Pa.). Growth was monitored by optical density (OD) at 600 nm. When the OD reached 0.5 units, iso-propyl-thio-β-D-galactoside (IPTG)(Sigma, St. Louis Mo.) was added to a final concentration of 1 mM to induce expression of Asd. Induction was continued for 3 hr, followed by harvesting of the cells by centrifugation and storage of the cell pellet at −20° C.

The Asd enzyme was purified from the cell pellets using a nickel affinity column. Approximately 30 mg of His-tagged protein was purified to homogeneity from a pellet from a 330 ml culture according to the following procedure. The cell pellet was resuspended in 20 ml of PO₄ buffer (0.054 g Na₂HPO_{4.7}H₂O, 0.028 g NaH₂PO₄.H₂O, 0.59 g NaCL, pH 7.4) containing 0.1% (w/v) Triton X 100 and 100 ng/μl of lysozyme and incubated at 25° C. for 25 min. The mixture was sonicated twice for 20 sec on ice after which it was centrifuged at 10,000×g for 10 min at 4° C. The supernatant was then passed through a 45 μm filter and run through a 5 ml HiTrap chelating nickel-charged column (Pharmacia, Pascataway N.J.) and washed with 5 column volumes of 20 mM sodium phosphate, pH 7.5. The nickel column was then eluted with 300 mM imidazole in PO₄ buffer. The nickel column eluate was subsequently dialyzed overnight against 100 mM Tris.HCl, pH 7.5. Glycerol was added to a final concentration of 25% (w/v) was added to the purified protein which was then stored at −20° C.

Development of an Assay for High-throughput Screening of Asd Enzymatic Characterization of H. Pylori Aspartate Semialdehyde Dehydrogenase (Asd)

The enzymatic activity of purified His-tagged H. pylori Asd (SEQ ID NO: 6545) was characterized by assaying the reverse reaction of phosphorylation of L-aspartate-β-semialdehyde (ASA) to L-p-aspartyl phosphate, with concomitant reduction of NADP⁺ to NADPH. The formation of NADPH was detected by its absorbance, 340 nm, or by its fluorescence, excitation =340 nm, emission 455 nm.

ASA was synthesized enzymatically by the phosphorylation of L-aspartate to L-β-aspartyl phosphate by aspartokinase (LysC) coupled to the reduction of L-β-aspartyl phosphate to L-aspartate-α-semialdehyde by Asd. The procedure involved a 2 h preincubation of 0.3 mg of purified E. coli LysC and E. coli Asd with 2 mM aspartate, 1 mM NADPH, 10 mM DTT, 30 mM ATP, 30 mM MgSO₄, 3 mM NH₄OH and 100 mM Tris-HCl buffer, pH 8, followed by adsorption of the enzymatic product on a column of the cation exchange resin Dowex 50 (hydrogen form, 400 mesh). The resin was washed with 5× columns of water and the aldehyde eluted in 5 ml fractions (1 ml/min) with 4 N HCL. Presence of the aldehyde was confirmed using the Asd enzymatic assay in the reverse direction and absence of aspartyl phosphate by the Asd enzymatic assay in the forward direction (Black et al., 1955, J. Biol. Chem. 213,39-50., Karsten et al., 1991, Biochim. Biophys. Acta 1077, 209-219). The identity of the compound was also confirmed by ¹³C and H⁺ NMR.

ASA in 4 N HCl was prepared daily for use in Asd assays by partial neutralization with addition of 1.0 equivalent of Na₂HCO3²⁻, with a resulting pH=1-2. The amount of ASA was determined enzymatically by complete reaction in the presence of excess NADP⁺and phosphate. ASA was found to be stable for up to 48 hours at pH 1.4, but is somewhat unstable at pH 7.7 (under assay conditions) with a t½=5 hrs. These observations are consistent with previous reports on the stability of ASA (Karsten et al., 1991, Biochim. Biophys. Acta 1077, 209-219). Assays were performed in the following buffer: 200 mM HEPES, 0.3 mM NADP⁺, 30 mM NaH₂PO₄, 1.0 mM DTT, pH 7.5 or pH 8.0. ASA was used in the assay at concentrations of 5-165 μM by diluting the partially-neutralized ASA stock into the assay 10-200 fold, leading to a decrease in pH of 0.1 units or less. Reaction was initiated with the addition of enzyme to a final concentration of 3.5-70 nM.

Using E. coli Asd, the Kₘ for ASA and phosphate were found to be 104 μM and 1.3 mM, respectively, with a k_{cat}=26 sec⁻¹ at pH 7.5. These values are roughly consistent with those reported by Karsten and Viola (1991, Biochim. Biophys. Acta 1077, 209-219) for the E. coli enzyme, considering the pH dependence of enzyme activity that was reported. For H. pylori Asd, we determined the Kₘ for ASA and phosphate to be 54 μM and 2.1 mM, respectively, with a k_{cat}=5.4 sec⁻¹ at pH 8.0. Thus, the H. pylori enzyme is similar to the E. coli enzyme with the exception of a decreased k_{cat} by about 5-fold. The pH dependence of H. pylori Asd activity, as measured by initial velocity, was found to be quite similar to that reported for the E. coli enzyme (Karsten et al., 1991, Biochim. Biophys. Acta 1077, 209-219), with a pH optimum between 8.5 and 9.0. The activity of H. pylori Asd was found to be unaffected by ionic strength as high as 0.7 M.

Based on the above characterization, high throughput drug screening assays can be performed in a 96-well plate format using a fluorescence microplate reader, detecting NADPH formation by measuring an increase in fluorescence with excitation filter =355 nm, emission filter =460 nm. Using this format with 160 μM ASA and 20-50 nM H. pylori Asd in assay buffer at pH 8.0, a simple 30 to 60 minute assay can be utilized with excellent reproducibility and high signal to noise.

The above described assays (Examples X and XI) could be further developed for screening chemical compounds for activity and the subsequent identification of a drug active against the target and cause inhibition of a bacterium, preferably H. pylori.

XII. Truncated Gene Expression and Protein Production

Identification, Cloning and Expression of Recombinant Helicobacter Pylori Sequences.

To facilitate the cloning, expression and purification of membrane proteins from H. pylori, the pET gene expression system (Novagen), for cloning and expression of recombinant proteins in Escherichia coli was selected. Further, for proteins that have a signal sequence at their amino-terminal end, a DNA sequence encoding a peptide tag (His-tag) was fused to the 5′ end of the H. pylori DNA sequences of interest in order to facilitate purification of the recombinant protein products. In some cases, the DNA sequence was cloned in frame with the glutathione-S-transferase protein to produce a GST-fusion protein. The vectors used in this case were the pGEX series from Pharmacia LKB (Uppsala, Sweden).

PCR Amplification and Cloning of DNA Sequences Containing ORFs for Membrane and Secreted Proteins from the J99 Strain of Helicobacter pylori.

The sequences chosen (from the list of the DNA sequences of the invention) for cloning from H. pylori strain J99 were prepared for amplification cloning by the polymerase chain reaction (PCR). Synthetic oligonucleotide primers for the ORF of interest (Table 15) specific for the predicted mature 5′ end of the ORF and either downstream (3′) of the predicted translational termination codon or at specific points within the coding region were designed and purchased (GibcoBRL Life Technologies, Gaithersburg, Md., USA). All forward primers (specific for the 5′ terminus of the region of ORF of interest) were designed to include either a BamHI or a NdeI restriction site. These primers within the NdeI restriction site sequence were designed to permit the initiation of protein translation at a methionine residue (encoded within the NdeI restriction site sequence, in the case of producing a non His-tagged recombinant protein) or to fuse in frame with the DNA sequence encoding the His-tag (for producing His tagged recombinant protein), followed by the coding sequence for the remainder of the native H. pylori DNA. The primer with the BamHI restriction site was produced to fuse the H. pylori specific sequence in-frame with the C-terminus of the glutathione-S-transferase gene in the pGEX vectors (Pharmacia LKB, Uppsala, Sweden). All reverse oligonucleotide primers designed to include an EcoRI restriction site at the 5′ terminus. Several reverse oligonucleotide primers were selected that would cause a truncation of the polypeptide to remove certain portions of the C-terminus, and in these cases the EcoRI restriction site at the 5′ end was followed by a translational termination codon. This combination of primers would enable the ORF of interest (or parts of the ORF of interest) to be cloned into pET28b (to produce a His-tagged recombinant protein), pET30a (to produce a non His tagged or native recombinant protein) or the pGEX-4T or pGEX-5× series (to produce a GST fusion protein). The pET28b vector provides sequence encoding an additional 20 amino-terminal amino acids (plus the methionine in the NdeI restriction site) including a stretch of six histidine residues which makes up the His-tag, whereas the pGEX vectors fuse the H. pylori protein to a 26,000 Da glutathione-S-transferase protein.

Genomic DNA prepared from H. pylori strain J99 (ATCC 55679) was used as the source of template DNA for the PCR amplification reactions (Current Protocols in Molecular Biology, John Wiley and Sons, Inc., F. Ausubel et al., eds., 1994). To amplify a DNA sequence containing a specific H. pylori ORF, genomic DNA (50 nanograms) was introduced into a reaction tube containing 200 nanograms of both the forward and reverse synthetic oligonucleotide primer specific for the ORF of interest, and 45 microliters of PCR SuperMix purchased (GibcoBRL Life Technologies, Gaithersburg, Md., USA) in a total of 50 microliters. The PCR SuperMix is supplied in 1.1× concentrations and contains 22 mM Tris-HCl (pH 8.4), 55 mM KCl, 1.65 mM MgCl₂, 220 micromolar of each dATP, dCTP, dGTP and dTTP, 22 units recombinant Taq polymerase/ml and stabilizers. The following thermal cycling conditions were used to obtain amplified DNA products for each ORF using a Perkin Elmer Cetus/GeneAmp PCR System thermal cycler.

TABLE 16
Oligonucleotide primers

| Gene and location | Sequence |
|---|---|
| Vac38- BamHI post signal sequence | CGGGATCCGAAGGTGATGGTGTTTAT |
| (SEQ ID NO: 360; SEQ ID NO: 5122) | ATAGG (SEQ ID NO: 10027) |
| Vac38- NdeI post signal sequence | CGCATATGGAAGGTGATGGTGTTTAT |
| | ATAGGG (SEQ ID NO: 10028) |
| Vac38- EcoRI/stop codon (removes C- | GCGAATTCTCACTCTTTCCAATAGTTT |
| terminal third of protein) | GCTGCAGAGC (SEQ ID NO: 10029) |
| Vac38- EcoRI/stop codon (removes C- | CCGGAATTCTTAATCCCGTTTCAAATG |
| terminal 11 amino acids) | GTAATAAAGG (SEQ ID NO: 10030) |
| Vac38- EcoRI downstream of native stop | GCGAATTCCCTTTTATTTAAAAAGTGT |
| codon | AGTTATACC (SEQ ID NO: 10031) |

Sequences for Vac38 (full length or truncated)
Denaturation at 94° C. for 30 sec
35 cycles at 94° C. for 15 sec, 55° C. for 15 sec, and 72° C. for 1.5 min
Reactions were concluded at 72° C. for 8 minutes

Upon completion of the thermal cycling reactions, each sample of amplified DNA was subjected to electrophoresis on 1.0% agarose gels. The DNA was visualized by exposure to ethidium bromide and long wave UV irradiation, and cut out in gel slices. DNA was purified using the Wizard PCR Preps kit (Promega Corp., Madison Wis., USA), and then subjected to digestion with BamHI and EcoRI (Current Protocols in Molecular Biology, John Wiley and Sons, Inc., F. Ausubel et al., eds., 1994). The digested PCR amplicon was then re-electrophoresed and purified as before.

Ligation of H. Pylori DNA Sequences into Cloning Vectors

The pOK12 vector (J. Vieira and J. Messing, Gene 100:189-194, 1991) was prepared for cloning by digestion with BamHI and EcoRI or NdeI and EcoRI in the case of Vac41 (Current Protocols in Molecular Biology, John Wiley and Sons, Inc., F. Ausubel et al., eds., 1994). The vectors were subjected to electrophoresis on 1.0% agarose gels and purified using the Wizard PCR Preps kit (Promega Corp., Madison Wis., USA). Following ligation of the purified, digested vector and the purified, digested amplified H. pylori ORF, the products of the ligation reaction were transformed into E coli JM109 competent cells according to standard methods (Current Protocols in Molecular Biology, John Wiley and Sons, Inc., F. Ausubel et al., eds., 1994). Individual bacterial colonies were screened for those containing the correct recombinant plasmids by incubating in LB broth overnight (plus 25 ug/ml kanamycin sulfate) followed by plasmid DNA preparation using the Magic Minipreps system (Promega Corp., Madison Wis., USA), and then analyzed by restriction digestion (Current Protocols in Molecular Biology, John Wiley and Sons, Inc., F. Ausubel et al., eds., 1994).

Cloning of H. Pylori DNA Sequences into the pET28b, pET30a and pGEX4T-3 Prokaryotic Expression Vectors

Both the pET28b and pET30a expression vectors were prepared for cloning by digestion with NdeI and EcoRI, and the pGEX4T-3 vector was prepared for cloning by digestion with BamHI and EcoRI (Current Protocols in Molecular Biology, John Wiley and Sons, Inc., F. Ausubel et al., eds., 1994). The H. pylori DNA sequences were removed from pOK 12 plasmid backbones by digestion with NdeI and EcoRI or BamHI and EcoRI (Current Protocols in Molecular Biology, John Wiley and Sons, Inc., F. Ausubel et al., eds., 1994). The pET28b, pET30a, pGEX4T-3 and H. pylori DNA sequences were all electrophoresed on a 1% agarose gel and purified using the Wizard PCR Preps kit (Promega Corp., Madison Wis., USA). Following ligation of the purified, digested expression vector and the purified, digested H. pylori DNA sequences, the products of the ligation reaction were transformed into E. coli JM109 competent cells (Current Protocols in Molecular Biology, John Wiley and Sons, Inc., F. Ausubel et al., eds., 1994). Individual bacterial colonies were screened for those containing the correct recombinant plasmids by preparing plasmid DNA as described above followed by analysis by restriction digestion profiles and DNA sequencing (Current Protocols in Molecular Biology, John Wiley and Sons, Inc., F. Ausubel et al., eds., 1994). These recombinant plasmids were then used to transform specific E coli expression strains.

Transformation of Competent Bacteria with Recombinant Expression Plasmids

Competent bacterial strains BL21 (DE3), BL21 (DE3)pLysS, HMS174(DE3) and HMS174(DE3)pLysS were prepared and transformed with the recombinant pET28b expression plasmids carrying the cloned H. pylori sequences according to standard methods (Current Protocols in Molecular Biology, John Wiley and Sons, Inc., F. Ausubel et al., eds., 1994). These expression host strains contain a chromosomal copy of the gene for T7 RNA polymerase. These hosts are lysogens of bacteriophage DE3, a lambda derivative that carries the lacI gene, the lacUV5 promoter and the gene for T7 RNA polymerase. T7 RNA polymerase expression is induced by the addition of isopropyl-β-D-thiogalactoside (IPTG), and the T7 RNA polymerase then transcribes any target plasmid, such as pET28b, that carries a T7 promoter sequence and a gene of interest.

Competent bacterial strains JM109 and DH5α were prepared and transformed with the recombinant pGEX4T-3 expression plasmid carrying the cloned H. pylori sequences according to standard methods (Current Protocols in Molecular Biology, John Wiley and Sons, Inc., F. Ausubel et al., eds., 1994).

Expression of Recombinant H. Pylori Sequences in E. Coli

Transformants were collected from LB agar plates containing 25 ug/ml kanamycin sulfate (ensures maintenance of the pET28b-based recombinant plasmids) or 100 ug/ml ampicillin (ensures maintenance of the pGEX4T-3-based recombinant plasmids) and used to inoculate LB broth containing 25 ug/ml kanamycin sulfate or 100 ug/ml ampicillin and grown to an optical density at 600 nm of 0.5 to 1.0 OD units, at which point 1 mM IPTG was added to the culture for one to three hours to induce gene expression of the H. pylori recombinant DNA constructions. After induction of gene expression with IPTG, bacteria were pelleted by centrifugation and resuspended in SDS-PAGE solubilization buffer and subjected to SDS-PAGE (Current Protocols in Molecular Biology, John Wiley and Sons, Inc., F. Ausubel et al., eds., 1994). Proteins were visualized by staining with Coomassie Brilliant Blue or detected by western immunoblotting using the specific anti-His tag monoclonal antibody (Clontech, Palo Alto, Calif., USA) or the anti-GST tag antibody (Pharmacia LKB) using standard methods (Current Protocols in Molecular Biology, John Wiley and Sons, Inc., F. Ausubel et al., eds., 1994). The host strain that provided the highest level of recombinant protein production was then chosen for use in a large-scale induction in order to purify the recombinant protein. The strains used were HMS174(DE3) (pET28b-based constructs) and DH5α (pGEX4T-3-based constructs).

Removal of the C-terminal regions appeared in both systems to improve the level of expression, although this increase was far more prominent in the GST-fusion system. All recombinant proteins produced were of the predicted molecular weight based on the DNA sequence plus, if necessary, the size of the fusion tag. The truncated portion of the H. pylori protein contains some extremely hydrophobic stretches, and removal of these may be the reason for the increased expression.

Equivalents

Those skilled in the art will recognize, or be able to ascertain using no more than routine experimentation, many equivalents to the specific embodiments and methods described herein. Such equivalents are intended to be encompassed by the scope of the following claims.

1. An isolated nucleic acid comprising an H. pylori nucleotide sequence selected from the group consisting of SEQ ID NO: 1-SEQ ID NO: 4762 and SEQ ID NO: 9525-SEQ ID NO: 9636.

2. An isolated nucleic acid comprising a nucleotide sequence encoding an H. pylori polypeptide at least about 60% homologous to an amino acid sequence selected from the group consisting of SEQ ID NO: 4763-SEQ ID NO: 9524 and SEQ ID NO: 9637-SEQ ID NO: 9748.

3. An isolated nucleic acid comprising a nucleotide sequence encoding an H. pylori polypeptide selected from the group consisting of SEQ ID NO: 4763-SEQ ID NO:9524 and SEQ ID NO: 9637-SEQ ID NO: 9748.

4. An isolated nucleic acid which encodes an H. pylori polypeptide, comprising a nucleotide sequence at least about 60% homologous to a nucleotide sequence selected from the group consisting of SEQ ID NO: 1-SEQ ID NO: 4762 and SEQ ID NO: 9525-SEQ ID NO: 9636, or a complement thereof.

5. The isolated nucleic acid of claim 2, comprising a nucleotide sequence selected from the group consisting of SEQ ID NO: 1-SEQ ID NO: 4762 and SEQ ID NO: 9525-SEQ ID NO: 9636, or a complement thereof.

6. An isolated nucleic acid molecule encoding an H. pylori polypeptide, comprising a nucleotide sequence which hybridizes under stringent hybridization conditions to a nucleic acid molecule comprising the nucleotide sequence selected from the group consisting of SEQ ID NO: 1-SEQ ID NO: 4762 and SEQ ID NO: 9525-SEQ ID NO: 9636, or a complement thereof.

7. An isolated nucleic acid comprising a nucleotide sequence encoding an H. pylori cell envelope polypeptide or a fragment thereof, the nucleic acid selected from the group consisting of SEQ ID NO: 1-SEQ ID NO: 1575 and SEQ ID NO: 9525-SEQ ID NO: 9592 or a complement thereof.

8. The isolated nucleic acid of claim 7, wherein the H. pylori cell envelope polypeptide or a fragment thereof is an H. pylori flagella-associated polypeptide or a fragment thereof encoded by a nucleic acid having a nucleotide sequence selected from the group consisting of SEQ ID NO: 1-SEQ ID NO: 103 and SEQ ID NO: 9525-SEQ ID NO: 9527, or a complement thereof.

9. The isolated nucleic acid of claim 7, wherein, the H. pylori cell envelope polypeptide or a fragment thereof is an H. pylori outer membrane polypeptide or a fragment thereof encoded by a nucleic acid having a nucleotide sequence selected from the group consisting of SEQ ID NO: 104-SEQ ID NO: 510 and SEQ ID NO: 9528-SEQ ID NO: 9536, or a complement thereof.

10. The isolated nucleic acid of claim 9, wherein the H. pylori outer membrane polypeptide or a fragment thereof is an H. pylori polypeptide having a terminal phenylalanine residue or a fragment thereof encoded by a nucleic acid having a nucleotide sequence selected from the group consisting of SEQ ID NO: 104-SEQ ID NO: 333; SEQ ID NO: 342-SEQ ID NO: 362; SEQ ID NO: 369-SEQ ID NO: 397 and SEQ ID NO: 9528-SEQ ID NO: 9536, or a complement thereof.

11. The isolated nucleic acid of claim 9, wherein the H. pylori outer membrane polypeptide or a fragment thereof is an H. pylori polypeptide having a C-terminal tyrosine cluster motif or a fragment thereof encoded by a nucleic acid having a nucleotide sequence selected from the group consisting of SEQ ID NO: 326-SEQ ID NO: 416; SEQ ID NO: 424-SEQ ID NO: 425 and SEQ ID NO: 9536, or a complement thereof.

12. The isolated nucleic acid of claim 10 or 11, wherein the H. Pylori outer membrane polypeptide or a fragment thereof is an H. pylori polypeptide having a terminal phenylalanine residue and a C-terminal tyrosine cluster or a fragment thereof encoded by a nucleic acid having a nucleotide sequence selected from the group consisting of SEQ ID NO: 326-SEQ ID NO: 333; SEQ ID NO: 342-SEQ ID NO: 362: SEQ ID NO; 369-SEQ ID NO: 397 and SEQ ID NO: 9536, or a complement thereof.

13. The isolated nucleic acid of claim 7, wherein the H. pylori cell envelope polypeptide or a fragment thereof is an H. pylori inner membrane polypeptide or a fragment thereof encoded by a nucleic acid having a nucleotide sequence selected from the group consisting of SEQ ID NO: 511-SEQ ID NO: 1514 and SEQ ID NO: 9537-SEQ ID NO: 9591, or a complement thereof.

14. The isolated nucleic acid of claim 13, wherein the H. pylori cell envelope polypeptide or a fragment thereof is an H. pylori inner membrane polypeptide or a fragment thereof involved in transport encoded by a nucleic acid having a nucleotide sequence selected from the group consisting of SEQ ID NO: 511-SEQ ID NO: 802; and SEQ ID NO: 9537-SEQ ID NO: 9554 or a complement thereof.

15. The isolated nucleic acid of claim 14, wherein the H. pylori cell envelope polypeptide or a fragment thereof is an H. pylori inner membrane polypeptide or a fragment thereof involved in amino acid metabolism and transport encoded by a nucleic acid having a nucleotide sequence selected from the group consisting of SEQ ID NO: 683-SEQ ID NO: 720 and SEQ ID NO: 9547-SEQ ID NO: 9548, or a complement thereof.

16. The isolated nucleic acid of claim 14, wherein the H. pylori cell envelope polypeptide or a fragment thereof is an H. pylori inner membrane polypeptide or a fragment thereof involved in nucleotide, lipid, or cofactor metabolism and transport encoded by a nucleic acid having a nucleotide sequence selected from the group consisting of SEQ ID NO: 721-SEQ ID NO: 764 and SEQ ID NO: 9549, or a complement thereof.

17. The isolated nucleic acid of claim 14, wherein the H. pylori cell envelope polypeptide or a fragment thereof is an H. pylori inner membrane polypeptide or a fragment thereof involved in inorganic ion transport encoded by a nucleic acid having a nucleotide sequence selected from the group consisting of SEQ ID NO: 765-SEQ ID NO: 799 and SEQ ID NO: 9550-SEQ ID NO: 9554, or a complement thereof.

18. The isolated nucleic acid of claim 14, wherein the H. pylori cell envelope polypeptide or a fragment thereof is an H. pylori inner membrane polypeptide or a fragment thereof involved in carbohydrate metabolism and transport encoded by a nucleic acid having a nucleotide sequence selected from the group consisting of SEQ ID NO: 800-SEQ ID NO: 802, or a complement thereof.

19. The isolated nucleic acid of claim 13, wherein the H. pylori cell envelope polypeptide or a fragment thereof is an H. pylori inner membrane polypeptide or a fragment thereof involved in outer membrane and cell wall formation encoded by a nucleic acid having a nucleotide sequence selected from the group consisting of SEQ ID NO: 803-SEQ ID NO: 848 and SEQ ID NO: 9555-SEQ ID NO: 9560, or a complement thereof.

20. The isolated nucleic acid of claim 13, wherein the H. pylori cell envelope polypeptide or a fragment thereof is an H. pylori inner membrane polypeptide or a fragment thereof involved in energy conversion encoded by a nucleic acid having a nucleotide sequence selected from the group consisting of SEQ ID NO: 849-SEQ ID NO: 995 and SEQ ID NO: 9561-SEQ ID NO: 9565, or a complement thereof.

21. The isolated nucleic acid of claim 13, wherein the H. pylori cell envelope polypeptide or a fragment thereof is an H. pylori inner membrane polypeptide or a fragment thereof involved in secretion and adhesion encoded by a nucleic acid having a nucleotide sequence selected from the group consisting of SEQ ID NO: 996-SEQ ID NO: 1007 and SEQ ID NO: 9566, or a complement thereof.

22. The isolated nucleic acid of claim 13, wherein the H. pylori cell envelope polypeptide or a fragment thereof is an H. pylori inner membrane polypeptide or a fragment thereof involved in regulation encoded by a nucleic acid having a nucleotide sequence selected from the group consisting of SEQ ID NO: 1008-SEQ ID NO: 1027, or a complement thereof.

23. The isolated nucleic acid of claim 13, wherein the H. pylori cell envelope polypeptide or a fragment thereof is an H. pylori inner membrane chaperone polypeptide or a fragment thereof encoded by a nucleic acid having a nucleotide sequence selected from the group consisting of SEQ ID NO: 1028-SEQ ID NO: 1031, and SEQ ID NO: 9567 or a complement thereof.

24. The isolated nucleic acid of claim 13, wherein the H. pylori cell envelope polypeptide or a fragment thereof is an H. pylori inner membrane polypeptide or a fragment thereof involved in cell division encoded by a nucleic acid having a nucleotide sequence selected from the group consisting of SEQ ID NO: 1032-SEQ ID NO: 1052 and SEQ ID NO: 9568-SEQ ID NO: 9569, or a complement thereof.

25. The isolated nucleic acid of claim 13, wherein the H. pylori cell envelope polypeptide or a fragment thereof is an H. pylori inner membrane polypeptide or a fragment thereof involved in motility encoded by a nucleic acid having a nucleotide sequence selected from the group consisting of SEQ ID NO: 1053; SEQ ID NO: 1054, or a complement thereof.

26. An isolated nucleic acid comprising a nucleotide sequence encoding an H. pylori cytoplasmic polypeptide or a fragment thereof, the nucleic acid selected from the group consisting of SEQ ID NO: 1576-SEQ ID NO: 4015 and SEQ ID NO: 9593-SEQ ID NO: 9621, or a complement thereof.

27. The isolated nucleic acid of claim 26, wherein the H. pylori cytoplasmic polypeptide or a fragment thereof is an H. pylori cytoplasmic polypeptide or a fragment thereof involved in metabolism encoded by a nucleic acid having a nucleotide sequence selected from the group consisting of SEQ ID NO: 1576-SEQ ID NO: 2218 and SEQ ID NO:9593-SEQ ID NO:9602.

28. The isolated nucleic acid of claim 27, wherein the H. pylori cytoplasmic polypeptide or a fragment thereof is an H. pylori polypeptide or a fragment thereof involved in energy metabolism encoded by a nucleic acid comprising a nucleotide sequence selected from the group consisting of SEQ ID NO: 1576-SEQ ID NO: 1725 and SEQ ID NO: 9593-SEQ ID NO: 9594, or a complement thereof.

29. The isolated nucleic acid of claim 27, wherein the H. pylori cytoplasmic polypeptide or a fragment thereof is an H. pylori polypeptide or a fragment thereof involved in amino acid metabolism encoded by a nucleic acid having a nucleotide sequence selected from the group consisting of SEQ ID NO: 1726-SEQ ID NO: 1868 and SEQ ID NO: 9595-SEQ ID NO: 9596, or a complement thereof.

30. The isolated nucleic acid of claim 27, wherein the H. pylori cytoplasmic polypeptide or a fragment thereof is an H. pylori polypeptide or a fragment thereof involved in cofactor metabolism encoded by a nucleic acid having a nucleotide sequence selected from the group consisting of SEQ ID NO: 1869-SEQ ID NO: 2005 and SEQ ID NO: 9597, or a complement thereof.

31. The isolated nucleic acid of claim 27, wherein the H. pylori cytoplasmic polypeptide or a fragment thereof is an H. pylori polypeptide or a fragment thereof involved in carbohydrate metabolism encoded by a nucleic acid having a nucleotide sequence selected from the group consisting of SEQ ID NO: 2006-SEQ ID NO. 2076 and SEQ ID NO: 9598-SEQ ID NO: 9599, or a complement thereof.

32. The isolated nucleic acid of claim 27, wherein the H. pylori cytoplasmic polypeptide or a fragment thereof is an H. pylori polypeptide or a fragment thereof involved in nucleic acid metabolism encoded by a nucleic acid having a nucleotide sequence selected from the group consisting of SEQ ID NO: 2077-SEQ ID NO: 2182 and SEQ ID NO: 9600, or a complement thereof.

33. The isolated nucleic acid of claim 27, wherein the H. pylori cytoplasmic polypeptide or a fragment thereof is an H. pylori polypeptide or a fragment thereof involved in lipid metabolism encoded by a nucleic acid having a nucleotide sequence selected from the group consisting of SEQ ID NO: 2183-SEQ ID NO: 2218 and SEQ ID NO: 9601-SEQ ID NO: 9602, or a complement thereof.

34. The isolated nucleic acid of claim 26, wherein the H. pylori cytoplasmic polypeptide or a fragment thereof is an H. pylori polypeptide or a fragment thereof involved in mRNA translation and ribosome biogenesis encoded by a nucleic acid having a nucleotide sequence selected from the group consisting of SEQ ID NO: 2219-SEQ ID NO: 2438 and SEQ ID NO: 9603-SEQ ID NO: 9604, or a complement thereof.

35. The isolated nucleic acid of claim 26, wherein the H. pylori cytoplasmic polypeptide or a fragment thereof is an H. pylori polypeptide or a fragment thereof involved in genome replication, transcription, recombination and repair encoded by a nucleic acid having a nucleotide sequence selected from the group consisting of SEQ ID NO: 2439-SEQ ID NO: 2807 and SEQ ID NO: 9605-SEQ ID NO: 9608, or a complement thereof.

36. The isolated nucleic acid of claim 26, wherein the H. pylori cytoplasmic polypeptide or a fragment thereof is an H. pylori polypeptide or a fragment thereof involved in secretion and adhesion encoded by a nucleic acid having a nucleotide sequence selected from the group consisting of SEQ ID NO: 2808-SEQ ID NO: 2824, or a complement thereof.

37. The isolated nucleic acid of claim 26, wherein the H. pylori cytoplasmic polypeptide or a fragment thereof is an H. pylori polypeptide or a fragment thereof involved in outer membrane and cell wall biogenesis encoded by a nucleic acid having a nucleotide sequence selected from the group consisting of SEQ ID NO: 2825-SEQ ID NO: 2877 and SEQ ID NO: 9609-SEQ ID NO: 9610, or a complement thereof.

38. The isolated nucleic acid of claim 26, wherein the H. pylori cytoplasmic polypeptide or a fragment thereof is an H. pylori polypeptide or a fragment thereof involved in protein folding and stabilization encoded by a nucleic acid having a nucleotide sequence selected from the group consisting of SEQ ID NO: 2878-SEQ ID NO:2918 and SEQ ID NO:9611-SEQ ID NO: 9612, or a complement thereof.

39. An isolated nucleic acid comprising a nucleotide sequence encoding an H. pylori secreted polypeptide or a fragment thereof, the nucleic acid selected from the group consisting of SEQ ID NO: 4016-SEQ ID NO: 4388 and SEQ ID NO: 9622-SEQ ID NO: 9625, or a complement therof.

40. The isolated nucleic acid of claim 39, wherein the H. pylori secreted polypeptide or a fragment thereof is an H. pylori periplasmic polypeptide or a fragment thereof encoded by a nucleic acid having a nucleotide sequence selected from the group consisting of SEQ ID NO: 4016-SEQ ID NO: 4018, or a complement thereof.

41. The isolated nucleic acid of claim 39, wherein the H. pylori secreted polypeptide or a fragment thereof is an H. pylori polypeptide or a fragment thereof involved in secretion and adhesion encoded by a nucleic acid having a nucleotide sequence selected from the group consisting of SEQ ID NO: 4019-SEQ ID NO: 4026 and SEQ ID NO: 9622, or a complement thereof.

42. The isolated nucleic acid of claim 39, wherein the H. pylori secreted polypeptide or a fragment thereof is an H. pylori chaperone polypeptide or a fragment thereof encoded by a nucleic acid having a nucleotide sequence selected from the group consisting of SEQ ID NO: 4027; SEQ ID NO: 4028-SEQ ID NO: 4030, or a complement thereof.

43. An isolated nucleic acid comprising a nucleotide sequence encoding an H. pylori cellular polypeptide or a fragment thereof, the nucleic acid selected from the group consisting of SEQ ID NO: 4389-SEQ ID NO: 4705 and SEQ ID NO: 9626-SEQ ID NO: 9636, or a complement thereof.

44. An isolated nucleic acid comprising a nucleotide sequence encoding an H. pylori membrane associated polypeptide or a fragment thereof, the nucleic acid selected from the group consisting of SEQ ID NO: 4706-SEQ ID NO: 4762, or a complement thereof.

45. An isolated H. pylori polypeptide comprising an amino acid sequence at least about 60% homologous to an H. pylori polypeptide selected from the group consisting of SEQ ID NO: 4763-SEQ ID NO: 9524 and SEQ ID NO: 9637-SEQ ID NO: 9748.

46. An isolated H. pylori polypeptide which is encoded by a nucleic acid comprising a nucleotide sequence at least about 60% homologous to a nucleotide sequence selected from the group consisting of SEQ ID NO: 1-SEQ ID NO:4762 and SEQ ID NO: 9525-SEQ ID NO: 9636.

47. The isolated H. pylori polypeptide of claim 46, wherein said polypeptide is encoded by a nucleotide sequence selected from the group consisting of SEQ ID NO: 1-SEQ ID NO: 4762 and SEQ ID NO: 9525-SEQ ID NO: 9636.

48. An isolated H. pylori polypeptide which is encoded by a nucleic acid comprising a nucleotide sequence which hybridizes under stringent hybridization conditions to a nucleic acid selected from the group consisting of SEQ ID NO: 1-SEQ ID NO: 4762 and SEQ ID NO: 9525-SEQ ID NO: 9636, or a complement thereof.

49. An isolated H. pylori polypeptide comprising an amino acid sequence selected from the group consisting of SEQ ID NO: 4763-SEQ ID NO: 9524 and SEQ ID NO: 9637-SEQ ID NO: 9748.

50. An isolated H. pylori cell envelope polypeptide or a fragment thereof, comprising an amino acid sequence selected from the group consisting of SEQ ID NO: 4763-SEQ ID NO: 6337 and SEQ ID NO: 9637-SEQ ID NO: 9704.

51. The isolated polypeptide of claim 50, wherein the H. pylori cell envelope polypeptide or a fragment thereof is an H. pylori flagella-associated polypeptide or a fragment thereof comprising an amino acid sequence selected from the group consisting of SEQ ID NO: 4763-SEQ ID NO: 4865 and SEQ ID NO: 9637-SEQ ID NO: 9704.

52. The isolated polypeptide of claim 50, wherein the H. pylori cell envelope polypeptide or a fragment thereof is an H. pylori outer membrane polypeptide or a fragment thereof comprising an amino acid sequence selected from the group consisting of SEQ ID NO: 4866-SEQ ID NO: 5272 and SEQ ID NO: 9640-SEQ ID NO: 9648.

53. The isolated polypeptide of claim 52, wherein the H. pylori outer membrane polypeptide or a fragment thereof is an H. pylori polypeptide having a terminal phenylalanine residue or a fragment thereof comprising an amino acid sequence selected from the group consisting of SEQ ID NO: 4866-SEQ ID NO: 5095; SEQ ID NO: 5104-SEQ ID NO: 5124; SEQ ID NO: 5131-SEQ ID NO: 5159 and SEQ ID NO: 9640-SEQ ID NO: 9648.

54. The isolated polypeptide of claim 52, wherein the H. pylori outer membrane polypeptide or a fragment thereof is an H. pylori polypeptide comprising a C-terminal tyrosine cluster motif or a fragment thereof having an amino acid sequence selected from the group consisting of SEQ ID NO: 5088-SEQ ID NO: 5178; SEQ ID NO: 5186-SEQ ID NO: 5187 and SEQ ID NO: 9648.

55. The isolated polypeptide of claim 53 or 54, wherein the H. pylori outer membrane polypeptide or a fragment thereof is an H. pylori polypeptide comprising a terminal phenylalanine residue and a C-terminal tyrosine cluster or a fragment thereof having an amino acid sequence selected from the group consisting of SEQ ID NO: 5088-SEQ ID NO: 5095; SEQ ID NO: 5104-SEQ ID NO: 5124; SEQ ID NO: 5131-SEQ ID NO: 5159 and SEQ ID NO: 9648.

56. The isolated polypeptide of claim 50, wherein the H. pylori cell envelope polypeptide or a fragment thereof is an H. pylori inner membrane polypeptide or a fragment thereof comprising an amino acid sequence selected from the group consisting of SEQ ID NO: 5273-SEQ ID NO: 6276 and SEQ ID NO: 9649-SEQ ID NO: 9703.

57. The isolated polypeptide of claim 56, wherein the H. pylori cell envelope polypeptide or a fragment thereof is an H. pylori inner membrane polypeptide or a fragment thereof involved in transport comprising an amino acid sequence selected from the group consisting of SEQ ID NO: 5273-SEQ ID NO: 5564; and SEQ ID NO: 9649-SEQ ID NO: 9666.

58. The isolated polypeptide of claim 57, wherein the H. pylori cell envelope polypeptide or a fragment thereof is an H. pylori inner membrane polypeptide or a fragment thereof involved in amino acid metabolism and transport comprising an amino acid sequence selected from the group consisting of SEQ ID NO: 5445-SEQ ID NO: 5482 and SEQ ID NO: 9659-SEQ ID NO: 9660.

59. The isolated polypeptide of claim 57, wherein the H. pylori cell envelope polypeptide or a fragment thereof is an H. pylori inner membrane polypeptide or a fragment thereof involved in nucleotide, lipid, or cofactor metabolism and transport comprising an amino acid sequence selected from the group consisting of SEQ ID NO: 5483-SEQ ID NO: 5526 and SEQ ID NO: 9661.

60. The isolated polypeptide of claim 57, wherein the H. pylori cell envelope polypeptide or a fragment thereof is an H. pylori inner membrane polypeptide or a fragment thereof involved in inorganic ion transport comprising an amino acid sequence selected from the group consisting of SEQ ID NO: 5527-SEQ ID NO: 5561 and SEQ ID NO: 9662-SEQ ID NO: 9666.

61. The isolated polypeptide of claim 57, wherein the H. pylori cell envelope polypeptide or a fragment thereof is an H. pylori inner membrane polypeptide or a fragment thereof involved in carbohydrate metabolism and transport comprising an amino acid sequence selected from the group consisting of SEQ ID NO: 5562-SEQ ID NO: 5564.

62. The isolated polypeptide of claim 56, wherein the H. pylori cell envelope polypeptide or a fragment thereof is an H. pylori inner membrane polypeptide or a fragment thereof involved in outer membrane and cell wall formation comprising an amino acid sequence selected from the group consisting of SEQ ID NO: 5565-SEQ ID NO: 5610 and SEQ ID NO: 9667-SEQ ID NO: 9672.

63. The isolated polypeptide of claim 56, wherein the H. pylori cell envelope polypeptide or a fragment thereof is an H. pylori inner membrane polypeptide or a fragment thereof involved in energy conversion comprising an amino acid sequence selected from the group consisting of SEQ ID NO: 5611-SEQ ID NO: 5757 and SEQ ID ND 9673-SEQ ID NO: 9677.

64. The isolated polypeptide of claim 56, wherein the H. pylori cell envelope polypeptide or a fragment thereof is an H. pylori inner membrane polypeptide or a fragment thereof involved in secretion and adhesion comprising an amino acid sequence selected from the group consisting of SEQ ID NO: 5758-SEQ ID NO: 5769 and SEQ ID NO: 9678.

65. The isolated polypeptide of claim 56, wherein the H. pylori cell envelope polypeptide or a fragment thereof is an H. pylori inner membrane polypeptide or a fragment thereof involved in regulation comprising an amino acid sequence selected from the group consisting of SEQ ID NO: 5770-SEQ ID NO: 5789.

66. The isolated polypeptide of claim 56, wherein the H. pylori cell envelope polypeptide or a fragment thereof is an H. pylori inner membrane chaperone polypeptide or a fragment thereof comprising an amino acid sequence selected from the group consisting of SEQ ID NO: 5790-SEQ ID NO: 5793 and SEQ ID NO: 9679.

67. The isolated polypeptide of claim 56, wherein the H. pylori cell envelope polypeptide or a fragment thereof is an H. pylori inner membrane polypeptide or a fragment thereof involved in cell division comprising an amino acid sequence selected from the group consisting of SEQ ID NO: 5794-SEQ ID NO: 5814 and SEQ ID NO: 9680-SEQ ID NO: 9681.

68. The isolated polypeptide of claim 56, wherein the H. pylori cell envelope polypeptide or a fragment thereof is an H. pylori inner membrane polypeptide or a fragment thereof involved in motility comprising an amino acid sequence selected from the group consisting of SEQ ID NO: 5815; SEQ ID NO: 5816.

69. An isolated H. pylori cell envelope polypeptide or a fragment thereof, wherein the polypeptide is encoded by a nucleic acid sequence selected from the group consisting of SEQ ID NO: 1-SEQ ID NO: 1575 and SEQ ID NO: 9525-SEQ ID NO: 9592.

70. The isolated polypeptide of claim 69, wherein the H. pylori cell envelope polypeptide or a fragment thereof is an H. pylori flagella-associated polypeptide or a fragment thereof encoded by a nucleic acid comprising a nucleotide sequence selected from the group consisting of SEQ ID NO: 1-SEQ ID NO: 103 and SEQ ID NO: 9525-SEQ ID NO: 9527.

71. The isolated polypeptide of claim 69, wherein the H. pylori cell envelope polypeptide or a fragment thereof is an H. pylori outer membrane polypeptide or a fragment thereof encoded by a nucleic acid comprising a nucleotide sequence selected from the group consisting of SEQ ID NO: 104-SEQ ID NO: 510 and SEQ ID NO: 9528-SEQ ID NO: 9536.

72. The isolated polypeptide of claim 71, wherein the H. pylori outer membrane polypeptide or a fragment thereof is an H. pylori polypeptide having a terminal phenylalanine residue or a fragment thereof encoded by a nucleic acid comprising a nucleotide sequence selected from the group consisting of SEQ ID NO: 104-SEQ ID NO: 333; SEQ ID NO: 342-SEQ ID NO: 362; SEQ ID NO: 369-SEQ ID NO: 397 and SEQ ID NO: 9528-SEQ ID NO: 9536.

73. The isolated polypeptide of claim 71, wherein the H. pylori outer membrane polypeptide or a fragment thereof is an H. pylori polypeptide having a C-terminal tyrosine cluster motif or a fragment thereof encoded by a nucleic acid comprising a nucleotide sequence selected from the group consisting of SEQ ID NO: 326-SEQ ID NO: 416; SEQ ID NO: 424-SEQ ID NO: 425 and SEQ ID NO: 9536.

74. The isolated polypeptide of claim 72 or 73, wherein the H. pylori outer membrane polypeptide or a fragment thereof is an H. pylori polypeptide having a terminal phenylalanine residue and a C-terminal tyrosine cluster or a fragment thereof encoded by a nucleic acid comprising a nucleotide sequence selected from the group consisting of SEQ ID NO: 326-SEQ ID NO: 333; SEQ ID NO: 342-SEQ ID NO: 362; SEQ ID NO: 369-SEQ ID NO: 397 and SEQ ID NO: 9536.

75. The isolated polypeptide of claim 69, wherein the H. pylori cell envelope polypeptide or a fragment thereof is an H. pylori inner membrane polypeptide or a fragment thereof encoded by a nucleic acid comprising a nucleotide sequence selected from the group consisting of SEQ ID NO: 511-SEQ ID NO: 1514 and SEQ ID NO: 9537-SEQ ID NO: 9591.

76. The isolated polypeptide of claim 75, wherein the H. pylori cell envelope polypeptide or a fragment thereof is an H. pylori inner membrane polypeptide or a fragment thereof involved in transport encoded by a nucleic acid comprising a nucleotide sequence selected from the group consisting of SEQ ID NO: 511-SEQ ID NO: 802; and SEQ ID NO: 9537-SEQ ID NO: 9554.

77. The isolated polypeptide of claim 76, wherein the H. pylori cell envelope polypeptide or a fragment thereof is an H. pylori inner membrane polypeptide or a fragment thereof involved in amino acid metabolism and transport encoded by a nucleic acid comprising a nucleotide sequence selected from the group consisting of SEQ ID NO: 683-SEQ ID NO: 720 and SEQ ID NO: 9547-SEQ ID NO: 9548.

78. The isolated polypeptide of claim 76, wherein the H. pylori cell envelope polypeptide or a fragment thereof is an H. pylori inner membrane polypeptide or a fragment thereof involved in nucleotide, lipid, or cofactor metabolism and transport encoded by a nucleic acid comprising a nucleotide sequence selected from the group consisting of SEQ ID NO: 721-SEQ ID NO: 764 and SEQ ID NO: 9549.

79. The isolated polypeptide of claim 76, wherein the H. pylori cell envelope polypeptide or a fragment thereof is an H. pylori inner membrane polypeptide or a fragment thereof involved in inorganic ion transport encoded by a nucleic acid comprising a nucleotide sequence selected from the group consisting of SEQ ID NO: 765-SEQ ID NO: 799 and SEQ ID NO: 9550-SEQ ID NO: 9554.

80. The isolated polypeptide of claim 76, wherein the H. pylori cell envelope polypeptide or a fragment thereof is an H. pylori inner membrane polypeptide or a fragment thereof involved in carbohydrate metabolism and transport encoded by a nucleic acid comprising a nucleotide sequence selected from the group consisting of SEQ ID NO: 800-SEQ ID NO: 802.

81. The isolated polypeptide of claim 75, wherein the H. pylori cell envelope polypeptide or a fragment thereof is an H. pylori inner-membrane polypeptide or a fragment thereof involved in outer membrane and cell wall formation encoded by a nucleic acid comprising a nucleotide sequence selected from the group consisting of SEQ ID NO: 803-SEQ ID NO: 848 and SEQ ID NO: 9555-SEQ ID NO: 9560.

82. The isolated polypeptide of claim 75, wherein In another embodiment, the H. pylori cell envelope polypeptide or a fragment thereof is an H. pylori inner membrane polypeptide or a fragment thereof involved in energy conversion encoded by a nucleic acid comprising a nucleotide sequence selected from the group consisting of SEQ ID NO: 849-SEQ ID NO: 995 and SEQ ID NO: 9561-SEQ ID NO: 9565.

83. The isolated polypeptide of claim 75, wherein the H. pylori cell envelope polypeptide or a fragment thereof is an H. pylori inner membrane polypeptide or a fragment thereof involved in secretion and adhesion encoded by a nucleic acid comprising a nucleotide sequence selected from the group consisting of SEQ ID NO: 996-SEQ ID NO: 1007 and SEQ ID NO: 9566.

84. The isolated polypeptide of claim 75, wherein the H. pylori cell envelope polypeptide or a fragment thereof is an H. pylori inner membrane polypeptide or a fragment thereof involved in regulation encoded by a nucleic acid comprising a nucleotide sequence selected from the group consisting of SEQ ID NO: 1008-SEQ ID NO: 1027.

85. The isolated polypeptide of claim 75, wherein the H. pylori cell envelope polypeptide or a fragment thereof is an H. pylori inner membrane chaperone polypeptide or a fragment thereof encoded by a nucleic acid comprising a nucleotide sequence selected from the group consisting of SEQ ID NO: 1028-SEQ ID NO: 1031 and SEQ ID NO: 9567.

86. The isolated polypeptide of claim 75, wherein the H. pylori cell envelope polypeptide or a fragment thereof is an H. pylori inner membrane polypeptide or a fragment thereof involved in cell division encoded by a nucleic acid having a nucleotide sequence selected from the group consisting of SEQ ID NO: 1032-SEQ ID NO: 1052 and SEQ ID NO: 9568-SEQ ID NO: 9569.

87. The isolated polypeptide of claim 75, wherein the H. pylori cell envelope polypeptide or a fragment thereof is an H. pylori inner membrane polypeptide or a fragment thereof involved in motility encoded by a nucleic acid comprising a nucleotide sequence selected from the group consisting of SEQ ID NO: 1053; SEQ ID NO: 1054.

88. An isolated H. pylori cytoplasmic polypeptide or a fragment thereof, wherein the polypeptide comprises amino acid sequence selected from the group consisting of SEQ ID NO: 6338-SEQ ID NO: 8777 and SEQ ID NO: 9705-SEQ ID NO: 9733.

89. The isolated polypeptide of claim 88, wherein the H. pylori cytoplasmic polypeptide or a fragment thereof is an H. pylori cytoplasmic polypeptide or a fragment thereof involved in metabolism comprising an amino acid sequence selected from the group consisting of SEQ ID NO: 6338-SEQ ID NO: 6945 and SEQ ID NO: 9705-SEQ ID NO: 9714.

90. The isolated polypeptide of claim 89, wherein the H. pylori cytoplasmic polypeptide or a fragment thereof is an H. pylori polypeptide involved in energy metabolism or a fragment thereof comprising an amino acid sequence selected from the group consisting of SEQ ID NO: 6338-SEQ ID NO: 6487 and SEQ ID NO: 9705-SEQ ID NO: 9706.

91. The isolated polypeptide of claim 89, wherein the H. pylori cytoplasmic polypeptide or a fragment thereof is an H. pylori polypeptide involved in amino acid metabolism or a fragment thereof comprising an amino acid sequence selected from the group consisting of SEQ ID NO: 6488-SEQ ID NO: 6630 and SEQ ID NO: 9707-SEQ ID NO: 9708.

92. The isolated polypeptide of claim 89, wherein the H. pylori cytoplasmic polypeptide or a fragment thereof is an H. pylori polypeptide involved in cofactor metabolism or a fragment thereof comprising an amino acid sequence selected from the group consisting of SEQ ID NO: 6631-SEQ ID NO: 6767 and SEQ ID NO: 9709.

93. The isolated polypeptide of claim 89, wherein the H. pylori cytoplasmic polypeptide or a fragment thereof is an H. pylori polypeptide involved in carbohydrate metabolism or a fragment thereof comprising an amino acid sequence selected from the group consisting of SEQ ID NO: 6768-SEQ ID NO: 6838 and SEQ ID NO: 9710-SEQ ID NO: 9711.

94. The isolated polypeptide of claim 89, wherein the H. pylori cytoplasmic polypeptide or a fragment thereof is an H. pylori polypeptide involved in nucleic acid metabolism or a fragment thereof comprising an amino acid sequence selected from the group consisting of SEQ ID NO: 6839-SEQ ID NO: 6944 and SEQ ID NO: 9712.

95. The isolated polypeptide of claim 89, wherein the H. pylori cytoplasmic polypeptide or a fragment thereof is an H. pylori polypeptide involved in lipid metabolism or a fragment thereof comprising an amino acid sequence selected from the group consisting of SEQ ID NO: 6945-SEQ ID NO: 6980 and SEQ ID NO: 9713-9714.

96. The isolated polypeptide of claim 88, wherein In another embodiment, the H. pylori cytoplasmic polypeptide or a fragment thereof is an H. pylori polypeptide involved in mRNA translation and ribosome biogenesis or a fragment thereof comprising an amino acid sequence selected from the group consisting of SEQ ID NO: 6981-SEQ ID NO: 7200 and SEQ ID NO: 9715-SEQ ID NO: 9716.

97. The isolated polypeptide of claim 88, wherein the H. pylori cytoplasmic polypeptide or a fragment thereof is an H. pylori polypeptide involved in genome replication, transcription, recombination and repair or a fragment thereof comprising an amino acid sequence selected from the group consisting of SEQ ID NO: 7201-SEQ ID NO: 7569 and SEQ ID NO: 9717-SEQ ID NO: 9720.

98. The isolated polypeptide of claim 88, wherein the H. pylori cytoplasmic polypeptide or a fragment thereof is an H. pylori polypeptide involved in secretion and adhesion or a fragment thereof comprising an amino acid sequence selected from the group consisting of SEQ ID NO: 7570-SEQ ID NO: 7586.

99. The isolated polypeptide of claim 88, wherein the H. pylori cytoplasmic polypeptide or a fragment thereof is an H. pylori polypeptide involved in outer membrane and cell wall biogenesis or a fragment thereof comprising an amino acid sequence selected from the group consisting of SEQ ID NO: 7587-SEQ ID NO: 7639 and SEQ ID NO: 9721-SEQ ID NO: 9722.

100. The isolated polypeptide of claim 88, wherein the H. pylori cytoplasmic polypeptide or a fragment thereof is an H. pylori polypeptide involved in protein folding and stabilization or a fragment thereof comprising an amino acid sequence selected from the group consisting of SEQ ID NO: 7640-SEQ ID NO: 7680 and SEQ ID NO: 9523-SEQ ID NO: 9724.

101. An isolated H. pylori cytoplasmic polypeptide or a fragment thereof, wherein the polypeptide is encoded by a nucleic acid sequence selected from the group consisting of SEQ ID NO: 1576-SEQ ID NO: 4015 and SEQ ID NO: 9593-SEQ ID NO: 9621.

102. The isolated polypeptide of claim 101, wherein the H. pylori cytoplasmic polypeptide or a fragment thereof is an H. pylori cytoplasmic polypeptide or a fragment thereof involved in metabolism encoded by a nucleic acid comprising a nucleotide sequence selected from the group consisting of SEQ ID NO: 1576-SEQ ID NO: 2218 and SEQ ID NO: 9593-SEQ ID NO: 9602.

103. The isolated polypeptide of claim 102, wherein the H. pylori cytoplasmic polypeptide or a fragment thereof is an H. pylori polypeptide involved in energy metabolism or a fragment thereof encoded by a nucleic acid comprising a nucleotide sequence selected from the group consisting of SEQ ID NO: 1576-SEQ ID NO: 1725 and SEQ ID NO: 9593-SEQ ID NO: 9594.

104. The isolated polypeptide of claim 102, wherein the H. pylori cytoplasmic polypeptide or a fragment thereof is an H. pylori polypeptide involved in amino acid metabolism or a fragment thereof encoded by a nucleic acid comprising a sequence selected from the group consisting of SEQ ID NO: 1726-SEQ ID NO: 1868 and SEQ ID NO: 9595-SEQ ID NO: 956.

105. The isolated polypeptide of claim 102, wherein the H. pylori cytoplasmic polypeptide or a fragment thereof is an H. pylori polypeptide involved in cofactor metabolism or a fragment thereof encoded by a nucleic acid comprising a sequence selected from the group consisting of SEQ ID NO: 1869-SEQ ID NO: 2005 and SEQ ID NO: 9597.

106. The isolated polypeptide of claim 102, wherein the H. pylori cytoplasmic polypeptide or a fragment thereof is an H. pylori polypeptide involved in carbohydrate metabolism or a fragment thereof encoded by a nucleic acid comprising a sequence selected from the group consisting of SEQ ID NO: 2006-SEQ ID NO: 2076 and SEQ ID NO: 9598-SEQ ID NO: 9599.

107. The isolated polypeptide of claim 102, wherein the H. pylori cytoplasmic polypeptide or a fragment thereof is an H. pylori polypeptide involved in nucleic acid metabolism or a fragment thereof encoded by a nucleic acid comprising a nucleotide sequence selected from the group consisting of SEQ ID NO: 2077-SEQ ID NO: 2182 and SEQ ID NO: 9600.

108. The isolated polypeptide of claim 102, wherein the H. pylori cytoplasmic polypeptide or a fragment thereof is an H. pylori polypeptide involved in lipid metabolism or a fragment thereof encoded by a nucleic acid comprising a nucleotide sequence selected from the group consisting of SEQ ID NO: 2183-SEQ ID NO: 2218 and SEQ ID NO: 9601-SEQ ID NO: 9602.

109. The isolated polypeptide of claim 101, wherein the H. pylori cytoplasmic polypeptide or a fragment thereof is an H. pylori polypeptide involved in mRNA translation and ribosome biogenesis or a fragment thereof encoded by a nucleic acid comprising a sequence selected from the group consisting of SEQ ID NO: 2219-SEQ ID NO: 2438 and SEQ ID NO: 9603-SEQ ID NO: 9604.

110. The isolated polypeptide of claim 101, wherein the H. pylori cytoplasmic polypeptide or a fragment thereof is an H. pylori polypeptide involved in genome replication, transcription, recombination and repair or a fragment thereof encoded by a nucleic acid comprising a nucleotide sequence selected from the group consisting of SEQ ID NO: 2439-SEQ ID NO: 2807 and SEQ ID NO: 9605-SEQ ID NO: 9608.

111. The isolated polypeptide of claim 101, wherein the H. pylori cytoplasmic polypeptide or a fragment thereof is an H. pylori polypeptide involved in secretion and adhesion or a fragment thereof encoded by a nucleic acid comprising a sequence selected from the group consisting of SEQ ID NO: 2808-SEQ ID NO: 2824.

112. The isolated polypeptide of claim 101, wherein the H. pylori cytoplasmic polypeptide or a fragment thereof is an H. pylori polypeptide involved in outer membrane and cell wall biogenesis or a fragment thereof encoded by a nucleic acid comprising a sequence selected from the group consisting of SEQ ID NO: 2825-SEQ ID NO: 2877 and SEQ ID NO: 9609-SEQ ID NO: 9610.

113. The isolated polypeptide of claim 101, wherein the H. pylori cytoplasmic polypeptide or a fragment thereof is an H. pylori polypeptide involved in protein folding and stabilization of a fragment thereof encoded by a nucleic acid comprising a sequence selected from the group consisting of SEQ ID NO:2878-SEQ ID NO: 2918 and SEQ ID NO: 9611-SEQ ID NO: 9612.

114. An isolated H. pylori secreted polypeptide or a fragment thereof, wherein the polypeptide comprises an amino acid sequence selected from the group consisting of SEQ ID NO: 8778-SEQ ID NO: 9150 and SEQ ID NO: 9734-SEQ ID NO: 9737.

115. The polypeptide of claim 114, wherein H. pylori secreted polypeptide or a fragment thereof is a H. pylori periplasmic polypeptide or a fragment thereof comprises an amino acid sequence selected from the group consisting of SEQ ID NO: 8778-SEQ ID NO: 8780.

116. The polypeptide of claim 114, wherein the H. pylori secreted polypeptide or a fragment thereof is a H. pylori polypeptide or a fragment thereof involved in secretion and adhesion comprising an amino acid sequence selected from the group consisting of SEQ ID NO: 8781-SEQ ID NO: 8788 and SEQ ID NO: 9734.

117. The polypeptide of claim 114, wherein the H. pylori secreted polypeptide or a fragment thereof is a H. pylori chaperone polypeptide or a fragment thereof comprising an amino acid sequence selected from the group consisting of SEQ ID NO: 8789-SEQ ID NO: 8792.

118. An isolated H. pylori secreted polypeptide or a fragment thereof, wherein the polypeptide is encoded by a nucleic acid sequence selected from the group consisting of SEQ ID NO: 4016-SEQ ID NO: 4388 and SEQ ID NO: 9622-SEQ ID NO: 9625.

119. The polypeptide of claim 118, wherein the H. pylori secreted polypeptide or a fragment thereof is a H. pylori periplasmic polypeptide or a fragment thereof encoded by a nucleic acid comprising a nucleotide sequence selected from the group consisting of SEQ ID NO: 4016-SEQ ID NO: 4018.

120. The polypeptide of claim 118, wherein the H. pylori secreted polypeptide or a fragment thereof is a H. pylori polypeptide or a fragment thereof involved in secretion and adhesion encoded by a nucleic acid comprising a sequence selected from the group consisting of SEQ ID NO: 4019-SEQ ID NO: 4026 and SEQ ID NO: 9622.

121. The polypeptide of claim 118, wherein the H. pylori secreted polypeptide or a fragment thereof is a H. pylori chaperone polypeptide or a fragment thereof encoded by a nucleic acid comprising a nucleotide sequence selected from the group consisting of SEQ ID NO: 4027-SEQ ID NO: 4030.

122. An isolated H. pylori cellular polypeptide or a fragment thereof, wherein the polypeptide comprises an amino acid sequence selected from the group consisting of SEQ ID NO: 9151-SEQ ID NO: 9467 and SEQ ID NO: 9738-SEQ ID NO: 9748.

123. An isolated H. pylori cellular polypeptide or a fragment thereof, wherein the polypeptide is encoded by a nucleic acid comprising a nucleotide sequence selected from the group consisting of SEQ ID NO: 4389-SEQ ID NO: 4705 and SEQ ID NO: 9626-SEQ ID NO: 9636.

124. An isolated H. pylori membrane associated polypeptide or a fragment thereof, wherein the polypeptide comprises an amino acid sequence selected from the group consisting of SEQ ID NO: 9468-SEQ ID NO: 9524.

125. An isolated H. pylori cellular polypeptide or a fragment thereof, wherein the polypeptide is encoded by a nucleic acid comprising a nucleotide sequence selected from the group consisting of SEQ ID NO: 4706-SEQ ID NO: 4762.

126. An isolated H. pylori chaperone polypeptide or a fragment thereof, wherein the polypeptide is encoded by a nucleic acid comprising a nucleotide sequence selected from the group consisting of SEQ ID NO: 1028-1031; SEQ ID NO: 9567; and SEQ ID NO: 4027-SEQ ID NO: 4030.

127. An isolated H. pylori chaperone polypeptide or a fragment thereof, wherein the polypeptide comprises an amino acid sequence selected from the group consisting of SEQ ID NO: 5790-5793; SEQ ID NO: 9679; and SEQ ID NO: 8789-SEQ ID NO: 8792.

128. An isolated nucleic acid comprising a nucleotide sequence encoding an H. pylori chaperone polypeptide or a fragment thereof, the nucleic acid selected from the group consisting of SEQ ID NO: 1028-SEQ ID NO: 1031 and SEQ ID NO: 9567 and SEQ ID NO: 4027-SEQ ID NO: 4030.

129. A chimeric H. pylori polypeptide comprising at least two H. pylori polypeptides or fragments thereof, wherein each polypeptide is encoded by a nucleic acid sequence of any one of claims 1, 2, 3, 4, 5, or 6.

130. A chimeric H. pylori polypeptide comprising at least two H. pylori polypeptides or fragments thereof, wherein each polypeptide comprises an amino acid sequence of any one of claims 45, 46, 47, 48 or 49.

131. A fusion protein comprising an H. pylori polypeptide which comprises an amino acid sequence selected from the group consisting of SEQ ID NO: 4763-SEQ ID NO: 9524 and SEQ ID NO: 9637-SEQ ID NO: 9748 operatively linked to a non-H. pylori polypeptide.

132. An isolated nucleic acid comprising a nucleotide sequence of at least 8 nucleotides in length, wherein the sequence hybridizes under stringent hybridization conditions to a nucleic acid having a nucleotide sequence selected from the group consisting of SEQ ID NO: 1-SEQ ID NO: 4762 and SEQ ID NO: 9525-SEQ ID NO: 9636, or a complement thereof.

133. A probe comprising a nucleotide sequence consisting of at least 8 nucleotides of a nucleotide sequence selected from the group consisting of SEQ ID NO: 1-SEQ ID 0: 4762 and SEQ ID NO: 9525-SEQ ID NO: 9636, or a complement thereof.

134. A recombinant expression vector comprising the nucleic acid of any of claims 1, 2, 3, 4, 6, 7, 26, 39, 42, 43, 44 operably linked to a transcription regulatory element.

135. A cell comprising a recombinant expression vector of claim 134.

136. A method for producing an H. pylori polypeptide comprising culturing a cell of claim 135 under conditions that permit expression of the polypeptide.

137. The method of claim 136, further comprising purifying the polypeptide from the cell.

138. A method for detecting the presence of a Helicobacter nucleic acid in a sample comprising:
(a) contacting a sample with a nucleic acid of any of claims 132 or 133 so that a hybrid can form between the probe and a Helicobacter nucleic acid in the sample; and
(b) detecting the hybrid formed in step (a), wherein detection of a hybrid indicates the presence of a Helicobacter nucleic acid in the sample.

139. A vaccine formulation for prophylactic or therapeutic treatment of an H. pylori infection comprising an effective amount of at least one isolated nucleic acid of any of claims 1, 2, 3, 4, 6, 7, 26, 39, 43 or 44.

140. A vaccine formulation for prophylactic or therapeutic treatment of an H. pylori infection comprising an effective amount of at least one H. pylori polypeptide or a fragment thereof of any of claims 45, 46, 48, 49, 50, 88, 114, 120, 122, or 124.

141. A vaccine formulation of claim 139, further comprising a pharmaceutically acceptable carrier.

142. A vaccine formulation of claim 140, further comprising a pharmaceutically acceptable carrier.

143. A vaccine formulation of claim 141, wherein the pharmaceutically acceptable carrier comprises an adjuvant.

144. A vaccine formulation of claim 142, wherein the pharmaceutically acceptable carrier comprises an adjuvant.

145. A vaccine formulation of claim 141, wherein the pharmaceutically acceptable carrier comprises a delivery system.

146. A vaccine formulation of claim 142, wherein the pharmaceutically acceptable carrier comprises a delivery system.

147. A vaccine formulation of claim 145, wherein the delivery system comprises a live vector.

148. A vaccine formulation of claim 146, wherein the delivery system comprises a live vector.

149. A vaccine formulation of claim 147, wherein the live vector is a bacteria or a virus.

150. A vaccine formulation of claim 148, wherein the live vector is a bacteria or a virus.

151. A vaccine formulation of claim 145, wherein the pharmaceutically acceptable carrier further comprises an adjuvant.

152. A vaccine formulation of claim 146, wherein the pharmaceutically acceptable carrier further comprises an adjuvant.

153. A method of treating or reducing a risk of H. pylori infection in a subject comprising administering to a subject a vaccine formulation of claim 139, such that treatment or reduction of risk of H. pylori infection occurs.

154. A method of treating or reducing a risk of H. pylori infection in a subject comprising administering to a subject a vaccine formulation of claim 140, such that treatment or reduction of risk of H. pylori infection occurs.

155. A method of producing a vaccine formulation comprising: combining at least one isolated H. pylori polypeptide or a fragment thereof, wherein each polypeptide or fragment thereof is an amino acid of any one of claims 45, 46, 48, 49, 50, 88, 114, 122, or 124.

156. A method of producing a vaccine formulation comprising:
(a) providing at least one isolated H. pylori polypeptide or a fragment thereof, wherein each polypeptide or fragment thereof is an amino acid of any one of claims 45, 46, 48, 49; 50; 88, 114, 122, or 124 and
(b) combining at least one said isolated H. pylori polypeptide or a fragment thereof with a pharmaceutically acceptable carrier to thereby form a vaccine formulation.

157. A method of producing a vaccine formulation comprising:
(a) culturing a cell under conditions that permit expression of an H. pylori polypeptide or a fragment thereof, wherein each polypeptide or fragment thereof is an amino acid of any one of claims 45, 46, 48, 49, 50, 88, 114, 122 or 124;
(b) isolating said H. pylori polypeptide or a fragment thereof from said cell; and
(c) combining at least one said isolated H. pylori polypeptide or a fragment thereof with a pharmaceutically acceptable carrier to thereby form a vaccine formulation.

158. A composition comprising at least one isolated nucleic acid encoding an H. pylori polypeptide or a fragment thereof of any of claims 1, 2, 3, 4, 6, 7, 26, 39, 43 or 44 and a pharmaceutically acceptable carrier.

159. A composition comprising at least one isolated H. pylori polypeptide or a fragment thereof of any of claims 45, 46, 48, 49, 50, 88, 114, 122, or 124.

160. A method of evaluating a compound for the ability to bind an H. pylori nucleic acid comprising: contacting said compound with an H. pylori nucleic acid of any one of claims 1, 2, 3, 4, 6, 7, 26, 39, 43 or 44; and determining if said compound binds said H. pylori nucleic acid.

161. A method of claim 160, wherein said compound is an activator of the bacterial life cycle.

162. A method of claim 160, wherein said compound is an inhibitor of the bacterial life cycle.

163. A method of claim 160, wherein said method is performed in vitro.

164. A method of claim 160, wherein said method is performed in vivo.

165. A method of evaluating a compound for the ability to bind an H. pylori polypeptide comprising: contacting said compound with an H. pylori polypeptide wherein said H. pylori polypeptide comprises an amino acid sequence of any one of claims 45, 46, 47, 48, or 49; and determining if said compound binds said H. pylori polypeptide.

166. A method of claim 165, wherein said compound is an activator of the bacterial life cycle.

167. A method of claim 165, wherein said compound is an inhibitor of the bacterial life cycle.

168. A method of claim 165, wherein said method is performed in vitro.

169. A method of claim 165, wherein said method is performed in vivo.

170. An isolated nucleic acid comprising a nucleotide sequence encoding an H. pylori murI polypeptide or a fragment thereof, said nucleic acid comprising the nucleotide sequence of SEQ ID NO: 2873.

171. A method of evaluating a compound for the ability to bind an H. pylori nucleic acid comprising: contacting said compound with an H. pylori nucleic acid of claim 170 and determining if said compound binds said H. pylori nucleic acid.

172. A method of claim 171, wherein said compound is an activator of the bacterial life cycle.

173. A method of claim 171, wherein said compound is an inhibitor of the bacterial life cycle.

174. A method of claim 171, wherein said method is performed in vitro.

175. The method of claim 171, wherein said method is performed in vivo.

176. A purified H. pylori murI polypeptide or a fragment thereof, wherein said polypeptide comprises the amino acid sequence of SEQ ID NO: 7635.

177 A method of evaluating a compound for the ability to bind an H. pylori polypeptide comprising: contacting said compound with an H. pylori polypeptide of claim 176 and determining if said compound binds said H. pylori polypeptide.

178. A method of claim 177, wherein said compound is an activator of the bacterial life cycle.

179. A method of claim 177, wherein said compound is an inhibitor of the bacterial life cycle.

180. A method of claim 177, wherein said method is performed in vitro.

181. The method of claim 177, wherein said method is performed in vivo.

182. An isolated nucleic acid comprising a nucleotide sequence encoding an H. pylori murC polypeptide or a fragment thereof, said nucleic acid comprising the nucleotide sequence of SEQ ID NO: 2845.

183. A method of evaluating a compound for the ability to bind an H. pylori nucleic acid comprising: contacting said compound with an H. pylori nucleic acid of claim 182 and determining if said compound binds said H. pylori nucleic acid.

184. A method of claim 183, wherein said compound is an activator of the bacterial life cycle.

185. A method of claim 183, wherein said compound is an inhibitor of the bacterial life cycle.

186. A method of claim 183, wherein said method is performed in vitro.

187. The method of claim 183, wherein said method is performed in vivo.

188. A purified H. pylori murC polypeptide or a fragment thereof, wherein said polypeptide comprises the amino acid sequence of SEQ ID NO: 7607.

189. A method of evaluating a compound for the ability to bind an H. pylori polypeptide comprising: contacting said compound with an H. pylori polypeptide of claim 188 and determining if said compound binds said H. pylori polypeptide.

190. A method of claim 189, wherein said compound is an activator of the bacterial life cycle.

191. A method of claim 189, wherein said compound is an inhibitor of the bacterial life cycle.

192. A method of claim 189, wherein said method is performed in vitro.

193. The method of claim 189, wherein said method is performed in vivo.

194. A vaccine formulation for prophylactic or therapeutic treatment of an H. pylori infection comprising an effective amount of at least one isolated nucleic acid encoding an H. pylori outer membrane polypeptide or a fragment thereof, said nucleic acid selected from the group consisting of SEQ ID NO: 212, SEQ ID NO: 254, SEQ ID NO: 205, SEQ ID NO: 329, SEQ ID NO: 384, SEQ ID NO: 377 and SEQ ID NO: 289.

195. The vaccine formulation of claim 194, wherein said nucleic acid comprises a nucleotide sequence of SEQ ID NO: 384.

196. A vaccine formulation for prophylactic or therapeutic treatment of an H. pylori infection comprising an effective amount of at least one H. pylori outer membrane polypeptide or a fragment thereof, said polypeptide selected from the group consisting of SEQ ID NO: 4974, SEQ ID NO: 5016, SEQ ID NO: 4967, SEQ ID NO: 5091, SEQ ID NO: 5146, SEQ ID NO: 5139 and SEQ ID NO: 5051.

197. The vaccine formulation of claim 196, wherein said H. pylori outer membrane polypeptide or a fragment thereof comprises the amino acid sequence of SEQ ID NO: 5146.

198. A vaccine formulation of claims 194 or 196, further comprising a pharmaceutically acceptable carrier.

199. A vaccine formulation of claim 198, wherein the pharmaceutically acceptable carrier comprises an adjuvant.

200. A vaccine formulation of claim 198, wherein the pharmaceutically acceptable carrier comprises a delivery system.

201. A vaccine formulation of claim 200, wherein the delivery system comprises a live vector.

202. A vaccine formulation of claim 201, wherein the live vector is a bacteria or a virus.

203. A vaccine formulation of claim 200, wherein the pharmaceutically acceptable carrier further comprises an adjuvant.

204. A vaccine formulation for prophylactic or therapeutic treatment of an H. pylori infection comprising an effective amount of at least one isolated nucleic acid encoding an H. pylori cell envelope polypeptide or a fragment thereof, said nucleic acid selected from the group consisting of SEQ ID NO: 469, SEQ ID NO: 89, SEQ ID NO: 4286, SEQ ID NO: 419, SEQ ID NO: 9618, and SEQ ID NO: 3253.

205. A vaccine formulation for prophylactic or therapeutic treatment of an H. pylori infection comprising an effective amount of at least one H. pylori cell envelope polypeptide or a fragment thereof, said polypeptide selected from the group consisting of SEQ ID NO: 5231, SEQ ID NO: 4851, SEQ ID NO: 9048, SEQ ID NO: 5181, SEQ ID NO: 9730, and SEQ ID NO: 8015.

206. A vaccine formulation of claims 204 or 205, further comprising a pharmaceutically acceptable carrier.

207. A vaccine formulation of claim 206, wherein the pharmaceutically acceptable carrier comprises an adjuvant.

208. A vaccine formulation of claim 206, wherein the pharmaceutically acceptable carrier comprises a delivery system.

209. A vaccine formulation of claim 208, wherein the delivery system comprises a live vector.

210. A vaccine formulation of claim 209, wherein the live vector is a bacteria or a virus.

211. A vaccine formulation of claim 208, wherein the pharmaceutically acceptable carrier further comprises an adjuvant.
